(19) United States
(12) Patent Application Publication
(10) Pub. No.: US 2023/0227576 A1
(43) Pub. Date: Jul. 20, 2023

(54) RECEPTORS PROVIDING TARGETED COSTIMULATION FOR ADOPTIVE CELL THERAPY

(71) Applicants: Instil Bio (UK) Limited, Manchester (GB); Instil Bio, Inc., Dallas, TX (US)

(72) Inventors: John Bridgeman, Manchester (GB); Robert Hawkins, Manchester (GB); Ruben Rodriguez, Dallas, TX (US); Gray Kueberuwa, Manchester (GB); Milena Kalaitsidou, Manchester (GB); Michael Gavin King, West Yorkshire (GB)

(21) Appl. No.: 18/157,027

(22) Filed: Jan. 19, 2023

Related U.S. Application Data
(63) Continuation-in-part of application No. 17/807,109, filed on Jun. 15, 2022.
(60) Provisional application No. 63/301,340, filed on Jan. 20, 2022.

(51) Int. Cl. C07K 16/30; C07K 7/06; C07K 14/715; C07K 14/725; A61K 35/17; C12N 5/0783

(52) U.S. Cl. CPC C07K 16/3007; C07K 7/06; C07K 14/715; C07K 14/7051; C07K 16/30; A61K 35/17; C12N 5/0636; A61K 39/00

(57) ABSTRACT

The present invention relates to a chimeric costimulatory antigen receptor (CoStAR) useful in adoptive cell therapy (ACT), and cells comprising the CoStAR. The CoStAR can act as a modulator of cellular activity enhancing responses to defined antigens. The present invention also provides CoStAR proteins, nucleic acids encoding the CoStAR and therapeutic uses thereof.

### RELATED APPLICATIONS AND INCORPORATION BY REFERENCE

This application is a continuation in part to U.S. application Ser. No. 17/807,109 filed Jun. 15, 2022, entitled “RECEPTORS PROVIDING TARGETED COSTIMULATION FOR ADOPTIVE CELL THERAPY,” and both this and Ser. No. 17/807,109 claims priority to U.S. Prov. App. No. 63/301,340 filed Jan. 20, 2022, entitled “RECEPTORS PROVIDING TARGETED COSTIMULATION FOR ADOPTIVE CELL THERAPY,” each of which is incorporated by reference in its entirety.

Reference is made to U.S. provisional patent applications 63/211,042 and 63/211,046, filed Jun. 16, 2021.

Reference is made to GB patent application Serial No. 1900858.0, filed 22 Jan. 2019, U.S. patent application Ser. No. 62/951,770, filed 20 Dec. 2019, International application PCT/GB2020/050120, filed 20 Jan. 2020, and U.S. provisional patent applications 63/053,494 and 63/053,498, filed Jul. 17, 2020.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### REFERENCE TO SEQUENCE LISTING

The present application is being filed along with a Sequence Listing in electronic format. The Sequence Listing is provided as a file entitled INSTB.008A.xml created on Jan. 18, 2023 which is 37,111,282 bytes in size. The information in the electronic format of the Sequence Listing is incorporated herein by reference in its entirety.

### FIELD OF THE INVENTION

The present invention relates to variants of a TRAF domain.

### BACKGROUND OF THE INVENTION

Adoptive cell therapy (ACT) using autologous T-cells to mediate cancer regression has shown much promise in early clinical trials. Several general approaches have been taken such as the use of naturally occurring tumor reactive or tumor infiltrating lymphocytes (TILs) expanded ex vivo. Additionally, T-cells may be genetically modified to retarget them towards defined tumor antigens. This can be done via the gene transfer of peptide (p)-major histocompatibility complex (MHC) specific T-cell Receptors (TCRs) or synthetic fusions between tumor specific single chain antibody fragment (scFv) and T-cell signaling domains (e.g. CD3ζ), the latter being termed chimeric antigen receptors (CARs).

TIL and TCR transfer has proven particularly good when targeting melanoma (Rosenberg et al. 2011; Morgan 2006), whereas CAR therapy has shown much promise in the treatment of certain B-cell malignancies (Grupp et al. 2013).

Costimulatory signals are useful to achieve robust CAR T cell expansion, function, persistence and antitumor activity. The success of CAR therapy in leukemia has been partly attributed to the incorporation of costimulatory domains (e.g. CD28 or CD137) into the CAR construct, signals from which synergize with the signal provided by CD3ζ to enhance anti-tumor activity. The basis of this observation relates to the classical signal 1/signal 2 paradigm of T-cell activation. Here signal 1, provided by the TCR complex, synergizes with signal 2 provided by costimulatory receptors such as CD28, CD137 or CD134 to permit the cells to undergo clonal expansion, IL2 production and long term survival without the activation induced cell death (AICD) associated with signal 1 alone. Furthermore the involvement of signal 2 enhances the signal generated through signal 1 allowing the cells to respond better to low avidity interactions such as those encountered during anti-tumor responses.

Targeted costimulation will have beneficial effects for non-CAR-based T-cell therapies. For example, incorporating costimulatory domains into a chimeric TCR has been shown to enhance responses of T-cells towards pMHC (Govers 2014). Further, there has been a move towards generating chimeric costimulatory receptors which separate the signal 2 generating costimulatory domain onto a separate receptor from the signal 1 generating CD3ζ (or other) moiety which can also reduce the chances of tonic signalling (Fisher et al 2019).

While tumor infiltrating lymphocytes (TILs) utilize their endogenous TCRs to mediate tumor recognition, it has not been possible to engineer the endogenous TCR. Thus TIL are subject to substantial limitations as tumor cells express very few costimulatory ligands. The ability to induce targeted costimulation of TIL, or indeed any other adoptive T-cell therapy product, would be beneficial.

Citation or identification of any document in this application is not an admission that such document is available as prior art to the present invention.

### SUMMARY OF THE INVENTION

The invention provides novel chimeric costimulatory antigen receptors (CoStARs) that bind to carcinoembryonic antigen (CEA) and cells comprising or expressing the CoStARs which are beneficial for CAR and non-CAR based T-cell therapies alike. The present invention uses cells that express a novel chimeric costimulatory receptor to provide a costimulatory signal to T-cells upon engagement with a defined disease-associated, for example tumor-associated, antigen.

There have been several reports in which split signal 1 and signal 2 have been used to drive antigen specific responses in engineered T-cells (Alvarez-Vallina & Hawkins 1996). However, none have utilized the full length CD28 molecule. There are specific advantages to using full length receptors, such as CD28 as opposed to truncated forms. A full length receptors may be capable of dimerization, enabling the receptor to function in its native form, indeed chimeric antigen receptors fail to function optimally when expressed as a monomer (Bridgeman et al. 2010).

In some embodiments, a CoStAR of the invention induces signal 2 upon engagement with a defined antigen such as a disease associated or tumor associated antigen. The full length CD28 molecule contains motifs critical to its native function in binding members of the B7 family of receptors; although this is potentially dangerous from the perspective of CARs carrying CD28 and CD3ζ receptors in tandem, wherein ligation of CAR by B7 could trigger T-cell activation, there are beneficial qualities for receptors harbouring signal 2 receptors alone. In an aspect, the invention provides a targeted chimeric costimulatory receptor (CoStAR) which comprises an extracellular binding domain operatively linked to a transmembrane domain, a first signaling domain, and a CD40 signaling domain or a signaling fragment thereof. The inventors have discovered that costimulatory receptors comprising a CD40 signaling domain display novel and improved activity profiles. The inventors have further discovered that variants of TRAF2, TRAF2/3, and TRAF6 binding domains within CD40 modulate signaling.

In certain embodiments of the invention, the CD40 signaling domain comprises SEQ ID NO:32, SEQ ID NO:33, or SEQ ID NO:34. In some embodiments, the CD40 signaling fragment comprises an SH3 motif (KPTNKAPH, SEQ ID NO:35), TRAF2 motif (PKQE, SEQ ID NO:36, PVQE, SEQ ID NO:37, SVQE, SEQ ID NO:38), TRAF6 motif (QEPQEINFP, SEQ ID NO:39), PKA motif (KKPTNKA, SEQ ID NO:40, SRISVQE, SEQ ID NO:41), or a combination thereof, or is a full length CD40 intracellular domain. In some embodiments, one or more of the SH3, TRAF2, TRAF6, or PKA motifs of the CD40 signaling domain is mutated.

In some embodiments, the first signaling domain of the CoStAR comprises a signaling domain or signaling fragment of a receptor, such as, for example a tumor necrosis factor receptor superfamily (TNFRSF) receptor, including but not limited to CD2, CD9, CD26, CD27, CD28, CD29, CD38, CD40, CD43, CD46, CD49d, CD55, CD73, CD81, CD82, CD99, CD100, CD134 (OX40), CD137 (41BB), CD150 (SLAM), CD270 (HVEM), CD278 (ICOS), DAP10, NTKR, CD357 (GITR), or EphB6. In some embodiments, the CoStAR comprises CD2, CD9, CD26, CD27, CD28, CD29, CD38, CD40, CD43, CD46, CD49d, CD55, CD73, CD81, CD82, CD99, CD100, CD134 (OX40), CD137 (41BB), CD150 (SLAM), CD270 (HVEM), CD278 (ICOS), DAP10, NTKR, CD357 (GITR), or EphB6. In embodiments, wherein the first signaling domain comprises a CD40 signaling domain thus the CoStAR comprises elements of two CD40 signaling domains.

In some embodiments, the CoStAR comprises a second signaling domain or signaling fragment of a receptor, such as, for example a tumor necrosis factor receptor superfamily (TNFRSF) receptor, including but not limited to CD2, CD9, CD26, CD27, CD28, CD29, CD38, CD40, CD43, CD46, CD49d, CD55, CD73, CD81, CD82, CD99, CD100, CD134 (OX40), CD137 (41BB), CD150 (SLAM), CD270 (HVEM), CD278 (ICOS), CD357 (GITR), or EphB6. The first signaling domain or signaling fragment, the CD40 signaling domain or signaling fragment, and the second signaling domain or signaling fragment can be in any order. Exemplary embodiments include, without limitation, CoStAR which comprise CD28, CD137, and CD40 signaling domains, CD28, CD134, and CD40 signaling domains, CD28, CD2, and CD40 signaling domains, CD28, GITR, and CD40 signaling domains, CD28, CD29, and CD40 signaling domains, or CD28, CD150, and CD40 signaling domains.

In some embodiments, the extracellular antigen-binding domain (e.g., without limitation CEA-binding domain, MSLN-binding domain) of a CoStAR of the invention is operatively linked to the transmembrane domain by a linker and/or a spacer. In some embodiments, the linker comprises from about 5 to about 20 amino acids. In some embodiments, the linker comprises AAAGSGGSG (SEQ ID NO:18).

In some embodiments, a CoStAR of the invention comprises a spacer which operatively links the extracellular binding domain to the transmembrane domain and comprises from about 10 to about 250 amino acids. In some embodiments, the spacer comprises an extracellular sequence of CD8 or CD28 or a fragment thereof. In some embodiments, the CoStAR comprises a second extracellular binding domain. In some embodiments, the second binding domain comprises an extracellular ligand binding domain from CD8 or CD28. In some embodiments, the spacer comprises one or more immunoglobulin domains or an immunoglobulin constant region. In some embodiments, the spacer comprises one or more immunoglobulin domains or an immunoglobulin constant region such as, without limitation, SEQ ID NO:24.

In some embodiments the transmembrane domain of a CoStAR of the invention comprises a transmembrane domain of a TNFRSF protein. In some embodiments, a transmembrane domain of a CoStAR of the invention comprises a transmembrane domain of CD28 or CD8. In some embodiments, a transmembrane domain of a CoStAR of the invention comprises a transmembrane sequence of CD28 or CD8.

The CoStARs of the invention are useful to stimulate an immune response against a selected target that expresses a tumor associated antigen (TAA), e.g. without limitation, carcinoembryonic antigen (CEA), mesothelin (MSLN), or other. In some embodiments, the CoStAR comprises an antigen binding fragment of the scFv of SEQ ID NO:9, SEQ ID NO:10, SEQ ID NO:11, SEQ ID NO:12, SEQ ID NO:13, or SEQ ID NO:14, e.g., a fragment comprising one, two, three, four, five, or all six complementary determining regions (CDRs). In some embodiments, the CoStAR comprises the scFv of SEQ ID NO:9, SEQ ID NO:10, SEQ ID NO:11, SEQ ID NO:12, SEQ ID NO:13, or SEQ ID NO:14. In some embodiments, the CoStAR comprises an antigen binding fragment of the scFv of SEQ ID NO:186, SEQ ID NO:187, SEQ ID NO:188, SEQ ID NO:189, SEQ ID NO:190, or SEQ ID NO:1191, e.g., a fragment comprising one, two, three, four, five, or all six CDRs. In some embodiments, the CoStAR comprises the scFv of SEQ ID NO:186, SEQ ID NO:187, SEQ ID NO:188, SEQ ID NO:189, SEQ ID NO:190, or SEQ ID NO:191.

According to the invention, an extracellular binding domain can comprise, without limit, an scFv, a peptide, an antigen binding portion of an antibody, an antibody heavy-chain variable domain, an antibody light chain variable domain, a single domain antibody, a CEA ligand, or an MSLN ligand.

In some embodiments, a CoStAR of the invention comprises a CD3ζ signaling domain, for example located at the C-terminus.

In some embodiments, a CoStAR of the invention comprises an N-terminal signal peptide. Non-limiting examples of N-terminal signal peptides are signal peptides of oncostatin M (OSM), CD8a, CD2, interleukin-2 (IL-2), granulocyte-macrophage colony stimulating factor (GM-CSF), and human IgGκ.

In an aspect of the invention, there is provided a nucleic acid which encodes a CoStAR of the invention. The nucleic acid may be optimized, for example be codon optimized for expression in a host cell. In a non-limiting embodiment, the nucleic acid is codon optimized for expression in a human cell.

In some embodiments, there is provided vector which encodes and is capable of expressing a CoStAR of any embodiment of the present disclosure.

In some embodiments, there is provided a cell which expresses a CoStAR of any embodiment of the present disclosure. In some embodiments, the cell expresses two or more CoStARs, for example the cell expresses a CoStAR that binds to CEA or MSLN and a CoStAR that binds to FOLR1 or a CoStAR that binds to CA125, such as but not limited to anti-CEA.CD28.CD40 and anti-CA125.41BB.CD40 or anti-MSLN.CD28.CD40 and anti-CA125.41BB.CD40. In some embodiments, the cell expresses a CoStAR which binds to CEA and a CoStAR which binds to PDL1, such as but not limited to anti-CEA.CD28.CD40 and PD1.CD28.CD40 or expresses a CoStAR which binds to MSLN and a CoStAR which binds to PDL1, such as but not limited to anti-MSLN.CD28.CD40 and PD1.CD28.CD40.

In some embodiments, a cell engineered to express a CoStAR of any embodiment of the present disclosure comprises an alpha-beta T cell, gamma-delta T cell, T regulatory cell, TIL, NKT cell or NK cell. In some embodiments, a cell engineered to express a CoStAR of any embodiment of the present disclosure coexpresses a chimeric antigen receptor (CAR) or a T cell receptor (TCR).

Some embodiments provide a method of making the cell which expresses a CoStAR which comprises transducing or transfecting a cell with a vector which encodes and is capable of expressing a CoStAR of any embodiment of the present disclosure.

Some embodiments provide a method for preparing a population of cells that express a CoStAR of any embodiment of the present disclosure by transducing or transfecting cells, detecting expression of the CoStAR and enriching, expanding, and/or selecting cells that express the CoStAR. In some embodiments, any of the CD40, TRAF2, TRAF2/3, or TRAF6 variants provided herein, including those in FIGS. 28, 67, 77A-77C, 83-89D, and 92 (excluding those in FIGS. 78 and 79), and including those in Tables 2, 4-5, and 7-14, and including SEQ ID NOS: 42, 475-479, 494-498, 505, 510-519, and 840-40839, and Consensus sequences A-C, can be used in the present method and/or construct and/or protein.

Some embodiments provide a method of treating a disease in a subject by administering a population of cells which express a CoStAR of any embodiment of the present disclosure. In some embodiments, the protein will comprise a variant CD40 and/or TRAF 2, 2/3, 6 domain as disclosed herein. In some embodiments, any of the CD40, TRAF2, TRAF2/3, or TRAF6 variants provided herein, including those in FIGS. 28, 67, 77A-77C, 83-89D, and 92 (excluding those in FIGS. 78 and 79), and including those in Tables 2, 4-5, and 7-14, and including SEQ ID NOS: 42, 475-479, 494-498, 505, 510-519, and 840-40839, and Consensus sequences A-C, can be used in the present method and/or construct and/or protein.

In some embodiments is a protein comprising an amino acid sequence comprising a sequence of Consensus A, or X₁X₂X₃X₄X₅X₆X₇X₈X₉X₁₀X₁₁, wherein: X₁ is any amino acid, X₂ is any amino acid, X₃ is P, X₄ is any amino acid, X₅ is Q, X₆ is C, E, or A, X₇ is any amino acid, X₈ is any amino acid, X₉ is any amino acid, X₁₀ is any amino acid, and X₁₁ is any amino acid. In some embodiments, the protein is part of a TRAF domain. In some embodiments, the protein is part of a TRAF2 domain. In some embodiments, the protein is part of a TRAF2/3 domain. In some embodiments, the sequence comprises: X₁ is S or P, X₂ is V, H, or Y, X₄ is I, Q, V, X₇ is T or S, X₈ is D, X₉ is K, D, or G, X₁₀ is T, S, G, or A, and X₁₁ is D, S, N, or D; or the sequence is at least 60% identical to a). In some embodiments, the protein is of the sequence GSNTX₁X₂X₃X₄X₅X₆X₇X₈X₉X₁₀X₁₁, SEQ ID NO: 42299. In some embodiments, the protein is of the sequence X₁X₂X₃X₄X₅X₆X₇X₈X₉X₁₀X₁₀QPVT, SEQ ID NO: 42377. In some embodiments, the protein is of the sequence GSNTX₁X₂X₃X₄X₅X₆X₇X₈X₉X₁₀X₁₁QPVT, SEQ ID NO: 42378.

In some embodiments is a protein comprising an amino acid sequence comprising a sequence of Consensus B, or X₁X₂X₃X₄X₅X₆X₇X₈X₉X₁₀X₁₁, wherein: X₁ is any amino acid, X₂ is any amino acid, X₃ is P, S, A, or T, X₄ is any amino acid, X₅ is Q or E, X₆ is E, X₇ is any amino acid, X₈ is any amino acid, X₉ is any amino acid, X₁₀ is any amino acid, and X₁₁ is any amino acid. In some embodiments, the protein is part of a TRAF domain. In some embodiments, the protein is part of a TRAF2 domain. In some embodiments, the protein is part of a TRAF2/3 domain. In some embodiments, the sequence comprises: X₁ is P, A, M, T, S, R, V, H, X₂ is F, A, L, I, T, V, G, C, A, F, P, X₄ is K, V, I, H, A, Q, T, E, S, X₇ is C, T, E, D, S, A, X₈ is A, L, G, Y, I, Q, D, E, X₉ is F, H, K, R, P, A, G, Y, E, N, X₁₀ is R, G, E, K, A, S, D, C, C, P, V, and X₁₁ is S, C, D, P, W, T, A, G, E; or the sequence is at least 60% identical to a). In some embodiments, the sequence is part of a Traf 2/3 sequence. In some embodiments, the protein is of the sequence GSNTX₁X₂X₃X₄X₅X₆X₇X₈X₉X₁₀X₁₁, SEQ ID NO: 42300. In some embodiments, the protein is of the sequence X₁X₂X₃X₄X₅X₆X₇X₈X₉X₁₀X₁₁QPVT, SEQ ID NO: 42379. In some embodiments, the protein is of the sequence GSNTX₁X₂X₃X₄X₅X₆X₇X₈X₉X₁₀X₁₁QPVT, SEQ ID NO: 42380.

In some embodiments is a protein comprising an amino acid sequence comprising a sequence of Consensus C, or X₁X₂X₃X₄X₅X₆, wherein: X₁ is P, X₂ is any amino acid, X₃ is E, X₄ is any amino acid, X₅ is any amino acid, and X₆ is Ac/Ar. In some embodiments, the protein is part of a TRAF domain. In some embodiments, the protein is part of a TRAF6 domain. In some embodiments, the sequence comprises: X₂ is Q, P, E, V, T, or S, X₄ is I, L M, N, S, T, N, D, X₅ is N, D, R, S, G, or Y; or the sequence is at least 60% identical to a). In some embodiments, the protein is of the sequence PKQEX₁X₂X₃X₄X₅X₆, SEQ ID NO: 42301. In some embodiments, the protein is of the sequence X₁X₂X₃X₄X₅X₆PDDL, SEQ ID NO: 42381. In some embodiments, the protein is of the sequence PKQEX₁X₂X₃X₄X₅X₆PDDL, SEQ ID NO: 42382.

In some embodiments is an amino acid sequence comprising: (a) one or more of SEQ ID NOs: 475, 476, 477, 478, 479, 494, 495, 496, 498, 505, 510, 511, 512, 513, 514, 515, 516, 517, 518, or 519; (b) a sequence that is at least 70% identical to a sequence in a); or (c) a sequence that is at least 80% similar to a).

In some embodiments is a TRAF domain in a CD40 protein, wherein the TRAF domain comprises one or more of: (a) SEQ ID NOs: 475, 476, 477, 478, 479, 494, 495, 496, 498, 505, 510, 511, 512, 513, 514, 515, 516, 517, 518, or 519; (b) a sequence that is at least 70% identical to a sequence in a); or (c) a sequence that is at least 80% similar to a).

In some embodiments is a TRAF6 domain sequence comprising: PQEINF, PEEMSW, PPENYE, or PQENSY.

In some embodiments is a TRAF2/3 domain sequence comprising: PVQET, TQEET, SKEET, AVEET, or PVQET.

In some embodiments is a TRAF2 domain sequence comprising: SVQE, AVEE or PEEE.

In some embodiments is a TRAF 6 or TRAF2/3 domain that comprises: (a) the amino acid sequence of one of SEQ ID NOs: 475, 476, 477, 478, 479, 494, 495, 496, 498, 505, 510, 511, 512, 513, 514, 515, 516, 517, 518, or 519; (b) the amino acid sequence that is at least 70% identical to a) or (c) the amino acid sequence that is at least 80% similar to a).

In some embodiments, the sequence is within a CD40 protein sequence for the protein, amino acid sequence, or TRAF domain of any one of the embodiments of the present discosure. In some embodiments, the sequence is within a CD40 protein and is located at a corresponding TRAF 2, TRAF2/3, and/or TRAF6 domain within the CD40 protein.

In some embodiments is a TRAF2/TRAF3 domain that comprises an amino acid sequence of Consensus A, or X₁X₂X₃X₄X₅X₆X₇X₈X₉X₁₀X₁₁, wherein X₁ is any amino acid, X₂ is any amino acid, X₃ is P, X₄ is any amino acid, X₅ is Q, X₆ is C, E, or A, X₇ is any amino acid, X₈ is any amino acid, X₉ is any amino acid, X₁₀ is any amino acid, and X₁₁ is any amino acid. In some embodiments, the protein is of the sequence GSNTX₁X₂X₃X₄X₅X₆X₇X₈X₉X₁₀X₁₁, SEQ ID NO: 42299. In some embodiments, the protein is of the sequence X₁X₂X₃X₄X₅X₆X₇X₈X₉X₁₀X₁₁QPVT, SEQ ID NO: 42377. In some embodiments, the protein is of the sequence GSNTX₁X₂X₃X₄X₅X₆X₇X₈X₉X₁₀X₁₁QPVT, SEQ ID NO: 42378.

In some embodiments is a TRAF2/TRAF3 domain that comprises an amino acid sequence of Consensus B, or X₁X₂X₃X₄X₅X₆X₇X₈X₉X₁₀X₁₁, wherein X₁ is any amino acid, X₂ is any amino acid, X₃ is P, S, A, or T, X₄ is any amino acid, X₅ is Q or E, X₆ is E, X₇ is any amino acid, X₈ is any amino acid, X₉ is any amino acid, X₁₀ is any amino acid, and X₁₁ is any amino acid. In some embodiments, the protein is of the sequence GSNTX₁X₂X₃X₄X₅X₆X₇X₈X₉X₁₀X₁₁, SEQ ID NO: 42300. In some embodiments, the protein is of the sequence X₁X₂X₃X₄X₅X₆X₇X₈X₉X₁₀X₁₁QPVT, SEQ ID NO: 42379. In some embodiments, the protein is of the sequence GSNTX₁X₂X₃X₄X₅X₆X₇X₈X₉X₁₀X₁₁QPVT, SEQ ID NO: 42380.

In some embodiments is a TRAF6 domain that comprises an amino acid sequence of Consensus C, or X₁X₂X₃X₄X₅X₆, wherein X₁ is P, X₂ is any amino acid, X₃ is E, X₄ is any amino acid, X₅ is any amino acid, and X₆ is Ac/Ar. In some embodiments, the protein is of the sequence PKQEX₁X₂X₃X₄X₅X₆, SEQ ID NO: 42301. In some embodiments, the protein is of the sequence X₁X₂X₃X₄X₅X₆PDDL, SEQ ID NO: 42381. In some embodiments, the protein is of the sequence PKQEX₁X₂X₃X₄X₅X₆PDDL, SEQ ID NO: 42382.

In some embodiments is a CD40 domain that comprises an amino acid sequence of one or more of the following: (a) TRAF2/TRAF3 domain that comprises an amino acid sequence of Consensus A, or X₁X₂X₃X₄X₅X₆X₇X₈X₉X₁₀X₁₁, wherein X₁ is any amino acid, X₂ is any amino acid, X₃ is P, X₄ is any amino acid, X₅ is Q, X₆ is C, E, or A, X₇ is any amino acid, X₈ is any amino acid, X₉ is any amino acid, X₁₀ is any amino acid, and X₁₁ is any amino acid. (b) a TRAF2/TRAF3 domain that comprises an amino acid sequence of Consensus B, or X₁X₂X₃X₄X₅X₆X₇X₈X₉X₁₀X₁₁, wherein X₁ is any amino acid, X₂ is any amino acid, X₃ is P, S, A, or T, X₄ is any amino acid, X₅ is Q or E, X₆ is E, X₇ is any amino acid, X₈ is any amino acid, X₉ is any amino acid, X₁₀ is any amino acid, and X₁₁ is any amino acid. (c) a TRAF6 domain that comprises an amino acid sequence of Consensus C, or X₁X₂X₃X₄X₅X₆, wherein X₁ is P, X₂ is any amino acid, X₃ is E, X₄ is any amino acid, X₅ is any amino acid, and X₆ is Ac/Ar.

In some embodiments is a chimeric costimulatory antigen receptor (CoStAR) which comprises: an extracellular binding domain that binds to carcinoembryonic antigen (CEA), or an extracellular binding domain that binds to mesothelin (MSLN); a transmembrane domain that is linked to the extracellular binding domain, a first signaling domain; and a second signaling domain, wherein the second signaling domain comprises a variant CD40 signaling domain or a signaling fragment thereof, wherein the variant CD40 comprises a sequence of a variant TRAF2/TRAF3 sequence, or a variant TRAF6 sequence, or a variant TRAF2 sequence, wherein the variant CD40 does not comprise SEQ ID NO: 42. In some embodiments, the first signaling domain comprises a signaling domain or signaling fragment of CD28 or CD278 (ICOS). In some embodiments, the construct is as shown in FIG. 88A. In some embodiments, the construct is as shown in FIG. 88B. In some embodiments, the construct is as shown in FIG. 88C. In some embodiments, the first signaling domain comprises a full-length costimulatory domain. In some embodiments, the extracellular binding domain is operatively linked to the transmembrane domain by a linker and/or a spacer. In some embodiments, the linker comprises from about 5 to about 20 amino acids. In some embodiments, the linker comprises from about 10 to about 250 amino acids. In some embodiments, the transmembrane domain comprises a transmembrane domain from CD28, CD8, ICOS, DAP10, or NTRK. In some embodiments, the transmembrane domain comprises the transmembrane domain sequence of SEQ ID NO:20, SEQ ID NO:21, or SEQ ID NO:22. In some embodiments, the extracellular binding domain comprises an scFv, a peptide, an antibody heavy-chain variable domain, an antibody light-chain variable domain, or a CEA ligand.

In some embodiments is a nucleic acid which encodes the CoStAR of any one of the embodiments of the present disclosure.

In some embodiments is a vector comprising the nucleic acid of any one of the embodiments of the present disclosure.

In some embodiments is a cell which expresses the CoStAR of any one of the embodiments of the present disclosure. In some embodiments, the cell comprises an alpha-beta T cell, gamma-delta T cell, T regulatory cell, TIL, NKT cell or NK cell. In some embodiments, the cell coexpresses a CAR or a TCR.

In some embodiments is a method of making the cell of any one of the embodiments of the present disclosure, which comprises the step of transducing or transfecting a cell with a vector of any one of the embodiments of the present disclosure

In some embodiments is a method for preparing a population of cells that express a CoStAR of any one the embodiments of the present disclosure, which comprises: detecting expression of the CoStAR on the surface of cells transfected or transduced with a vector of alternative 62; and selecting cells which are identified as expressing the CoStAR.

In some embodiments is a cell population which is enriched for cell expression a CoStAR of any one of the embodiments of the present disclosure.

In some embodiments is a method for treating a disease in a subject, which comprises the step of administering a cell according to any one of the embodiments of the present disclosure, or a cell population according to any one of the embodiments of the present disclosure, to the subject.

In some embodiments is a protein comprising the amino acid sequence of one of SEQ ID NOs: 510-519. In some embodiments, the protein comprises the sequence of SEQ ID NO: 510. In some embodiments, the protein comprises the sequence of SEQ ID NO: 511. In some embodiments, the protein comprises the sequence of SEQ ID NO: 512. In some embodiments, the protein comprises the sequence of SEQ ID NO: 513. In some embodiments, the protein comprises the sequence of SEQ ID NO: 514. In some embodiments, the protein comprises the sequence of SEQ ID NO: 515. In some embodiments, the protein comprises the sequence of SEQ ID NO: 516. In some embodiments, the protein comprises the sequence of SEQ ID NO: 517. In some embodiments, the protein comprises the sequence of SEQ ID NO: 518. In some embodiments, the protein comprises the sequence of SEQ ID NO: 519. In some embodiments, the protein comprises any of the constructs as shown in FIG. 92 (CTP188-CTP202).

Accordingly, it is an object of the present disclosure not to encompass within the present disclosure any previously known product, process of making the product, or method of using the product such that Applicants reserve the right and hereby disclose a disclaimer of any previously known product, process, or method. It is further noted that the present disclosure does not intend to encompass within the scope of the disclosure any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the present disclosure to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. patent law; e.g., they can mean “includes”, “included”, “including”, and the like; and that terms such as “consisting essentially of” and “consists essentially of” have the meaning ascribed to them in U.S. patent law, e.g., they allow for elements not explicitly recited, but exclude elements that are found in the prior art or that affect a basic or novel characteristic of any of the embodiments of the present disclosure.

These and other embodiments are disclosed or are obvious from and encompassed by, the following Detailed Description.

### BRIEF DESCRIPTION OF THE DRAWINGS

The following detailed description, given by way of example, but not intended to limit any embodiment of the present invention solely to the specific embodiments described, may best be understood in conjunction with the accompanying drawings.

FIG. 1—Structural organisation of single costimulatory and fusion costimulatory domain receptors. A schematic representation of CoStAR receptors set out in the claims is shown. First a CoStAR based on a single costimulatory receptor, and secondly a fusion CoStAR consisting of a full length costimulatory receptor signalling domain fused to a second costimulatory domain.

FIGS. 2Ai-2E—Genomic organisation of potential CoStAR configurations—The CoStAR consists of an antigen binding domain, an optional spacer domain and a costimulatory domain as shown in figure and described in claims. The CoStAR may be expressed: (FIGS. 2Ai-2Aii) alone from a promoter with the CoStAR consisting of a single (FIG. 2Ai) or fusion (FIG. 2Aii) costimulatory receptor; (FIG. 2B) may be expressed with an epitope tag (e.g. His tag, DYKDDDDK (SEQ ID NO:449) etc) at the N or C-terminus to enable direct staining of the CoStAR; (FIG. 2C) along with a marker gene separated using a 2A cleavage sequence or internal ribosomal entry site (IRES); ((FIG. 2D) along with a marker gene which is expressed from a second promoter; (FIG. 2E) along with a protein of interest such as a chimeric antigen receptor or T-cell receptor separated using a 2A cleavage sequence or internal ribosomal entry site (IRES); (FIG. 2F) along with a protein of interest such as a chimeric antigen receptor or T-cell receptor which is expressed from a second promoter. It would be clear to an individual with sufficient knowledge that the CoStAR and marker gene/chimeric antigen receptor/T-cell receptor/other protein of interest could be expressed in either orientation or 3′ (3-prime) or 5′ (5-prime) to one another.

FIGS. 3A-3E—Functional activity of CoStAR in T-cells in response to LS174T and LoVo tumour presented antigen. Normal donor T-cell populations from donor 1 (FIGS. 3A & 3D), donor 2 (FIG. 3B) and donor 3 (FIGS. 3C & 3E) were lentivirally engineered to express a CoStAR which targets carcinoembryonic antigen and magnetically sorted to enrich for the transgene using CD34 magnetic selection. T-cells were mixed with wild-type un-engineered CEA+ tumour cells (Non-activating tumour) or CEA+ tumour cells engineered to express a cell surface anchored anti-CD3 single chain antibody fragment (Activating tumour) at the indicated effector to target ratios and IL-2 measured in the supernatant by ELISA. Data obtained using LS174T cells (FIGS. 3A, 3B & 3C) and LoVo (3D & 3E).

FIGS. 4A-4D—Effect of CoStAR on T-cell proliferation. 5×10⁵ transduced and non transduced T-cells were mixed with 6.25×10⁴ wild-type LoVo or LoVo-OKT3 cells in the presence (FIG. 4A) or absence (FIG. 4B) of IL-2 and cell counts made after three days. In another assay under the same cell ratios T-cells from two donors (FIGS. 4C and 4D) were loaded with proliferation dye and the number of proliferation cycles the cells had gone through determined by dye dilution after six days using flow cytometry.

FIG. 5—IL-2 activity of CoStAR fusion receptors in primary human T-cells. Normal donor CD8+ T-cells from seven donors (except control CoStAR is three donors) were lentivirally transduced with the indicated CEA-targeting CoStARs and IL-2 production assessed after an overnight stimulation in the presence of LoVo-OKT3 cells. The proportion of IL-2 positive cells was determined using intracellular flow staining in both the CD34 negative (CoStAR non-transduced) and CD34+(CoStAR transduced) populations. Asterisks show significant differences between the transduced and non-transduced populations using paired Wilcoxon signed rank test with * p<0.05

FIGS. 6A-6D—Multi parameter analysis of CoStAR activity in primary human T-cells. Normal donor CD8+ T-cells were lentivirally transduced with the indicated CEA-targeting CoStARs and IL-2 production assessed after an overnight stimulation in the presence of LoVo-OKT3 cells. The proportion of IL-2 (seven donors) (FIG. 6A), IFNγ (seven donors) (FIG. 6B), BCL-xL (five donors) (FIG. 6C) and CD107a (six donors) (FIG. 6D) positive cells was determined using intracellular flow staining in both the CD34 negative (CoStAR non-transduced) and CD34+(CoStAR transduced) populations. Control is a non-specific CA125 targeting CoStAR and is from three donors in all instances. Heat maps are averages of all donors with the intensity of colour related to the percentage of cells positive for a particular read out under the defined conditions.

FIG. 7—CD40 enhances IL-2 production from CD28-based CoStARs. Primary human T-cells from three healthy donors were left non-transduced or transduced with either extracellular domain truncated CD28 (Tr CD28), full length CD28 (FL CD28), or CD28.CD40-based CoStARs harbouring a CEA specific scFv (MFE23). Transduced cells were selected using a CD34 marker gene and expanded prior to analysis. T-cells were mixed at an 8:1 effector to target ratio with OKT3 expressing CEA+ LoVo cells for 20 hours before analysis of IL-2 production by ELISA.

FIG. 8—Effect of signalling domain and target antigen on CoStAR-mediated T-cell expansion. T-cells were transduced with either DYKDDDDK (SEQ ID NO:449) epitope-tagged CD28 or CD28.CD40 based CoStARs harbouring CA125, FolR or CEA specific scFv, or FolR specific binding peptide (C7). T-cells were mixed with OKT3 expressing, CA125+/FolR+/CEA-cell line OVCAR3. The number of transduced cells were counted every 7 days up to 21 days, with fresh OVCAR3 cells added following each count.

FIG. 9—Effect of signalling domain and target antigen on CoStAR− mediated T-cell expansion. T-cells were transduced with either DYKDDDDK (SEQ ID NO:449) epitope-tagged CD28 or CD28.CD40 based CoStARs harbouring CA125, FolR or CEA specific scFv, or FolR specific binding peptide (C7). T-cells were mixed with OKT3 expressing CA125+/FolR+/CEA-cell line OVCAR3. The number of transduced cells were counted every 7 days up to 21 days, with fresh OVCAR3 cells added following each count.

FIGS. 10A-10B—(FIGS. 10A and 10B) CD40 based CoStARs enhance costimulation of T-cells in a model of TCR-transfer. Primary human T-cells from three healthy donors were transduced with a CEA specific TCR plus either a DYKDDDK-tagged CD28 or CD28.CD40 based CoStAR harbouring either an MFE (open or closed circles) or CA125 (open squares) specific scFv. T-cells were mixed at a 1:1 effector:target ratio with CEA+/CA125−H508 cells and intracellular cytokine staining performed to determine the number of responding CD4+ or CD8+ T-cells in the TCR+/CoStAR+, TCR+/CoStAR−, TCR−/CoStAR+ and TCR−/CoStAR− populations. A 2-way ANOVA (Tukeys test) was performed to determine significant differences in activity: *p>0.05, ** p>0.01, *** p>0.001, **** p>0.0001.

FIG. 11 depicts enrichment and expansion of primary human T-cells transduced to express costimulatory molecules of the invention. MFE23 is a single chain Fv antibody that has a high affinity for carcinoembryonic antigen (CEA). Primary human T-cells were mock transduced or transduced with MFE23.CD28 or MFE23.CD28.CD40 CoStAR, each harboring a CD34 marker gene separated by a 2A cleavage peptide. Following in vitro culture cells were enriched for CD34 using MACS™ paramagnetic selection reagents (Miltenyi Biotech) and then the cells expanded in number using irradiated feeder cells. Exemplary plots from one of three donors are shown.

FIGS. 12A-12D depict expansion of T-cells transduced with costimulatory molecules of the invention in response to stimulation and exogenous IL-2. Cells were mock transduced or transduced with MFE23.CD28 or MFE23.CD28.CD40 CoStAR and cocultured with LoVo-OKT3 cells at an 8:1 effector:target ratio in the presence (200 IU/ml) or absence of exogenous IL-2. At days 1, 4, 7, 11 and 18 cells were taken and the number of viable T-cells enumerated by using anti-CD2 reagents on a MACSQuant flow cytometer. (FIG. 12A) In the absence of stimulation by tumor and IL-2 cells declined in number as would be expected. (FIG. 12B) In the absence of stimulation but presence of IL-2 there was a more apparent survival of the cells, but no specific growth. (FIG. 12C) In the presence of tumor, but absence of IL-2 mock cells did not show specific survival. MFE23.CD28 CoStAR mediated an apparent doubling in expansion over the first four days followed by decline. MFE23.CD28.CD40 mediated a greater expansion up to day 7 followed by a steady decline. (FIG. 12D) Under the same conditions but in the presence of IL-2 both mock and MFE23.CD28 transduced cells demonstrated a 20-fold expansion over 18 days, whereas MFE23.CD28.CD40 cells expanded by over 60-fold. Thus CD28.CD40 based receptors demonstrate superior expansion and survival under conditions of stimulation both in the presence and absence of exogenous IL-2.

FIGS. 13A-13M depict cytokine production by mock, MFE23.CD28 or MFE23.CD28.CD40 engineered T-cells. Bead array analysis was performed on supernatants obtained from T-cell/tumour cocultures. Engineered T-cells were incubated at a 1:1 effector:target ratio with LoVo-OKT3 cells for 24 hours and supernatant collected. Conditioned supernatant was also collected from an equal number of T-cells alone, or LoVo-OKT3 cells alone. Cytokine production was analysed using a Legendplex™ Human TH1/TH2 cytokine panel (Biolegend). (FIG. 13A) IL-2; (FIG. 13B) IFN-7; (FIG. 13C) TNFα; (FIG. 13D) IL-4; (FIG. 13E) IL-5; (FIG. 13F) IL-13; (FIG. 13G) IL-17A; (FIG. 13H) IL-17F; (FIG. 13I) IL-22; (FIG. 13J) IL-6; (FIG. 13K) IL-10; (FIG. 13L) IL-9; (FIG. 13M) IL-21. Cytokines were either very low or undetectable in media from T-cells or tumour alone. When cocultured with tumor, cytokine production was enhanced. MFE23.CD28 enhanced production of IL-2, IL-5, IL-17A/17F, IL-10, IL-9 and IL-21 compared to mock. MFE23.CD28.CD40 also enhanced production of TNFα, IL-13 and IL-22. MFE23.CD28.CD40 and further enhanced the production of a number of cytokines greater than that provided by MFE23.CD28 (IL-2, IL-9 and IL-17F), as well as reducing the production of some cytokines below the levels seen with MFE23.CD28 (IL-5 and IL-10). Together this data demonstrates that addition of CD40 to CD28-based costimulatory receptors enhances and/or modulates their specific activity with respect to chemokine production.

FIGS. 14A-14M depict an analysis of chemokines using a Legendplex™ Human Pro inflammatory chemokine panel. (FIG. 14A) IL-8 (CXCL8); (FIG. 14B) IP-10 (CSCL10); (FIG. 14C) Eotaxin (CCL11); (FIG. 14D) TARC (CCL17); (FIG. 14E) MCP-1 (CCL2); (FIG. 14F) RANTES (CCL5); (FIG. 14G) MIP-1a (CCL3) (FIG. 14H) MIG (CXCL9) (FIG. 14I) ENA-78 (CXCL5) (FIG. 14J) MIP-3a (CCL20) (FIG. 14K) GROα (CXCL1) (FIG. 14L) I-TAC (CXCL11) (FIG. 14M) MEP-13 (CCL4). Chemokines were either very low or undetectable in media from T-cells alone. When cocultured with tumor, chemokine production was enhanced. MFE23.CD28 enhanced production of CXCL5, CXCL10, CXCL11, CCL17 and CCL20 compared to mock. MFE23.CD28.CD40 also enhanced production of CCL2, CXCL1 and CXCL9. MFE23.CD28.CD40 further enhanced the production of a number of cytokines greater than that provided by MFE23.CD28 (CXCL1, CXCL9, CXCL10, CXCL11, CCL17, CCL2, CXCL9, CCL5 and CCL20), as well as reducing the production of some cytokines below the levels seen with MFE23.CD28 (CCL4). Together this data demonstrates that addition of CD40 to CD28-based costimulatory receptors enhances and/or modulates their specific activity with respect to chemokine production.

FIGS. 15A-15H depict functional activity of ovarian CoStAR engineered cells using a CoStAR harbouring a FolR or CA125 reactive scFv (MOV19 & 196-14 respectively). Human folate receptor alpha (FolR) represents a suitable target for a number of tumours including ovarian, head and neck, renal and lung and CA125 represents an alternative target for ovarian cancer. Primary human T-cells from six healthy donors were engineered with either 196-14.CD28, 196-14.CD28.CD40, MOV19.CD28 or MOV19.CD28.CD40 receptors, all harbouring a DYKDDDDK epitope tag for detection. Transduced cells were mixed with FolR+/CA125+ OVCAR-OKT3 cells before analysis of effector activity using intracellular staining in the epitope tag positive and negative populations. Specific enhancement of effector activity determined by production of IL-2 (FIGS. 15A and 15B), TNFα (FIGS. 15C and 15D), CD137 (FIGS. 15E and 15F), and BCL-xL (FIGS. 15G and 15H) was observed in in CD28 and CD28.CD40 engineered cells in response to both CA125 and FolR, except for specific BCL-xL induction by MOV19.CD28 which was not observed compared to MOV19.CD28.CD40.

FIGS. 16A-16F depict three TIL populations mock transduced or engineered with MOV19.CD28.CD40 CoStAR and then mixed with patient matched tumor digest. The donor tumors displayed varying levels of FolR on the digest, ranging from negative (FIG. 16A), low expression (FIG. 16B) to high expression (FIG. 16C). Mock and CoStAR negative TIL in the CoStAR engineered populations of TIL matched for the FolR negative digest demonstrated similar levels of CD137 upregulation following tumor coculture which was not enhanced by the presence of CoStAR (FIG. 16D). In the TIL exposed to FolR low expressing digest there was an enhancement in activity in the CoStAR+ cells compared to CoStAR−, with CD137 expression increasing from <10% to >20% (FIG. 16E). In the TIL exposed to FolR high tumor digest there was an increase in activity from around 20% in the CoStAR− population, up to approximately 50% in the CoStAR+ population (FIG. 16F).

FIGS. 17A-17C depict enhancement of effector functions. A FolR targeting CoStAR enhanced CD137 expression from ˜20% to ˜50% (FIG. 17A), TNFα production from 10% to 15% (FIG. 17B) and IL-2 production from 2% to 5%. (FIG. 17C) in response to FolR+ tumor digest.

FIGS. 18A-18F depict soluble ligand does not inhibit effector functions. T-cells from three healthy donors were engineered with MOV19.CD28 or MOV19.CD28.CD40 CoStAR and activated with either immobilised OKT3, providing stimulation in the absence of FolR, or with OvCAR-OKT3, to provide TCR and CoStAR activity. BCL-xL activity was increased from between 10 and 20% across the three donors following OKT3 stimulation (FIG. 18A) whereas IL-2 was increased between 0 and 12% (FIG. 18B) and TNFα increased between 0 and 20% (FIG. 18C). The presence of exogenous soluble FolR did not enhance any of these particular effector functions. In the presence of OvCAR-OKT3 BCL-XL induction was enhanced by ˜20% in CD28 CoStAR but by ˜35% in CD28.CD40 CoStAR (FIG. 18D), IL-2 induction was enhanced by ˜20% in CD28 CoStAR but 30-50% in CD28.CD40 CoStAR (FIG. 18E) and TNFα production was enhanced by 20-30% in CD28 CoStAR and 25-50% in CD28.CD40 CoStAR (FIG. 18F). Exogenous soluble FolR did not have an inhibitory effect on any of these effector functions.

FIG. 19 depicts surface expression of anti-MSLN CoStAR expression on the surface of HD T cells. Transduced and non-transduced (MOCK) cells underwent a rapid expansion protocol (REP) and were assessed for transduction efficiency either via surface detection of the marker gene tCD34 (truncated CD34) or CoStAR molecule using an anti-CD34-APC (black) or anti-MSLN-PE (red) antibody, respectively. The results represent 3 biological replicates.

FIGS. 20A-20C depict cytokine expression in healthy donor (HD) T cells transduced with anti-MSLN CoStARs and cocultured with OVCAR-3 cell lines. CoStARs comprising combinations of six different anti-MSLN scFvs (SS1, M5, HN1, M912, huYP218, P4) and three different spacer/transmembrane domains (CD28, N-terminal truncated CD28, CD8) were compared. Structural details are provided in Table 8, Table 9, and Table 10. Cytokine concentrations for (FIG. 20A) IL-2 (FIG. 20B) IFNγ and (FIG. 20C) TNFα following cocultures with OVCAR-3 or OVCAR3-OKT3 cell lines are shown. Non-treated T cells were used as a control. The results represent 1-3 biological replicates with 3 technical replicates each.

FIGS. 21A-21C depict cytokine expression in healthy donor (HD) T cells transduced with anti-MSLN CoStARs and cocultured with K562 cell lines. CoStARs comprising combinations of six different anti-MSLN scFvs (SS1, M5, HN1, M912, huYP218, P4) and three different spacer/transmembrane domains (CD28, N-terminal truncated CD28, CD8) were compared. Structural details are provided in Table 8, Table 9, and Table 10. Cytokine concentrations for (FIG. 21A) IL-2 (FIG. 21B) IFNγ and (FIG. 21C) TNFα following cocultures with K562-MSNL or K562-MSNL-OKT3 cell lines are shown. Non-treated T cells were used as a control. The results represent 1-3 biological replicates with 3 technical replicates each.

FIG. 22 depicts surface expression of MFE23 scFV anti-CEA CoStARs expressed with six different secretion signal peptides (OSM1, CD8, CD2, IL2, GMCSF, hIgGκ). Structural details are provided in Table 11. Following expansion, cells were assessed for transduction efficiency either via surface detection of the marker gene truncated CD34 (tCD34) or CoStAR molecule using an anti-CD34-APC (black bars) or using a primary rhCEACAM5-Fc antibody with a secondary anti-IgG-Fc-PE (grey bars) antibody, respectively.

FIG. 23 depicts surface expression of CoStARs comprising six different anti-CEA scFvs (MFE23, MFE23(Q>K), hMFE23, CEA6, BW431/26, hT84.66). Structural details are provided in Table 12. Following expansion, cells were assessed for transduction efficiency either via surface detection of the marker gene truncated CD34 (tCD34) or CoStAR molecule using an anti-CD34-APC (black bars) or using a primary rhCEACAM5-Fc antibody with a secondary anti-IgG-Fc-PE (grey bars) antibody, respectively.

FIGS. 24A-24C depict cytokine production by healthy donor (HD) T cells transduced with anti-CEA CoStARs (MFE23, MFE23(Q>K), hMFE23, CEA6, BW431/26, hT84.66) and cocultured with LoVo cell lines. Structural details are provided in Table 12. Cytokine concentrations are shown for (FIG. 24A) IL-2 (FIG. 24B) IFNγ and (FIG. 24C) TNFα following cocultures with Lovo or Lovo-OKT3 cell lines. Non-treated T cells were used as a negative control.

FIGS. 25A-25C depict cytokine production in healthy donor (HD) T cells transduced with anti-CEA CoStARs (MFE23, MFE23(Q>K), hMFE23, CEA6, BW431/26, hT84.66) and cocultured with K562 cell lines. Cytokine concentrations are shown for (FIG. 25A) IL-2 (FIG. 25B) IFNγ and (FIG. 25C) TNFα following cocultures with K562.CEACAM5 (signal 2) or K562.CEACAM5.OKT3 (signal 1+2) cell lines. Non-treated T cells were used as a negative control.

FIG. 26 depicts surface expression of hMFE23 scFV anti-CEA CoStARs expressed with three different spacer/transmembrane domains. Structural details are provided in Table 13. Following expansion, cells were assessed for transduction efficiency via surface detection of the marker gene truncated CD34 (tCD34) using an anti-CD34-APC (black bars) or the CoStAR molecule using a primary rhCEACAM5-Fc antibody with a secondary anti-IgG-Fc-PE antibody (grey bars).

FIGS. 27A-27C depict cytokine production by the MFE23 scFV anti-CEA spacer variants. Cytokine concentrations for (FIG. 27A) IL-2 (FIG. 27B) IFNγ and (FIG. 27C) TNFα following cocultures with Lovo or Lovo.OKT3 cell lines are shown. Non-treated T cells were used as a control.

FIG. 28 depicts anti-CEA CoStARs comprising an hMFE23 CEA-binding domain with intracellular signalling domains comprising CD40, CD134, CD137, CD2, ICOS, DAP10 and NTRK1 signaling elements.

FIG. 29 depicts surface expression of hMFE23 scFV anti-CEA CoStARs comprising domain combinations depicted in FIG. 28 and detail in Table 14. Following expansion, cells were assessed for transduction efficiency via surface detection of the marker gene truncated CD34 (tCD34) using an anti-CD34-APC (black circles) or the CoStAR molecule using a primary rhCEACAM5-Fc antibody with a secondary anti-IgG-Fc-PE antibody (red circles). The results represent three biological replicates.

FIG. 30 depicts T cell phenotypes of CoStAR transfected HD T cells in three separate donors. Cells were sorted using CD34 microbeads and underwent a rapid expansion protocol (REP) for 14 days. Following outgrowth and REP, 1×10⁵ cells were assessed for the differentiation subtype using flow cytometry. Tcm, central memory T cell; Tem, effector memory T cell; Tn, naïve T cell; Tscm; stem cell memory T cell; Tte, terminal effector T cell. See Table 15 for T cell subtype definitions.

FIGS. 31A-31C depict cytokine production by hMFE23 scFV anti-CEA CoStAR transduced HD T cells cocultured with K562 cell lines. Cytokine concentrations for (FIG. 31A) IL-2, (FIG. 31B) IFNγ, and (FIG. 31C) TNFα are shown following cocultures with K562.CEACAM5 (signal 2) or K562.CEACAM5.OKT3 (signal 1+2) cell lines. Non-treated T cells were used as a control.

FIGS. 32A-32C depict cytokine expression in HD T cells transduced with hMFE23 scFV anti-MSLN CoStARs and cocultured with K562 cell lines. Frequency of (FIG. 32A) IL-2, (FIG. 32B) IFNγ, and (FIG. 32C) TNFα expressing cells is shown following cocultures with K562.CEACAM5 (signal 2) or K562.CEACAM5.OKT3 (signal 1+2) cell lines. Non-treated T cells were used as a control.

FIG. 33 depicts proliferation of HD T cells from transduced with hMFE23 scFV anti-MSLN CoStARs cocultured with K562.CEACAM5.OKT3 (signal 1+2) cell lines. HD T cells were procured from two donors. The figures represent fold expansion of input cells.

FIG. 34 depicts proliferation of HD T cells from transduced with hMFE23 scFV anti-MSLN CoStARs cocultured with K562.CEACAM5.OKT3 (signal 1+2) cell lines. HD T cells were procured from two donors. The figures represent fold expansion of input cells on Day 6 post stimulation.

FIGS. 35A-35B depict TRAF and TRAF-like binding sites and motifs and signalling pathways. FIG. 35A depicts a CoStAR CD40 intracellular domain showing TRAF binding sites and signalling. FIG. 35B depicts a CoStAR comprising an IProx domain.

FIGS. 36A-36B depict the effect of mutations in CoStAR CD40 intracellular signaling domain on cytokine secretion and long-term survival and proliferation of CD28.CD40 CoStAR transduced T cells cocultured with LoVo.OKT3. Cells of three donors were activated with Dynabeads and transduced with WT CD28.CD40 (CTP194), CD28.CD40 containing TRAF2 binding site mutation SVQE>AVQA (CTP195), TRAF2/TRAF3 binding site mutation PVQET>AVAEA (CTP196), TRAF6 binding site mutation PQEINF>AQAINF (CTP197), Q263A (CTP199), or mock transduced. (FIG. 36A) IL-2 was measured in supernatants collected 24 hours after coculture in absence of IL-2 with LoVo or LoVo.OKT3.GFP tumor cells. (FIG. 36B) Viability and absolute count were assessed after 6-8 days and live T cells were rechallenged for an additional week with fresh LoVo.OKT3.GFP tumor cells. At the end of the long-term coculture, the viability and absolute count were measured, and the fold expansion was calculated. Data shown as mean+/−SEM of n≤3 donors analysed in triplicates.

FIGS. 37A-37B depict percentage of TIL (FIG. 37A) and total TIL counts (FIG. 37B) based on CD2+ stain in thawed OC samples at day 1.

FIG. 38 depicts growth of non-Td and Td TILs. Cells were counted using CD2 and DRAQ7 staining and acquisition on the Novocyte 3005. Cell counts at the end of REP on day 25 are graphically represented. Shapes corresponding to the each of the 5 ovarian cancer samples are depicted. Filled shapes are Non-Td and open shapes are Td TILs. Statistical analysis was performed using a paired t-test.

FIGS. 39A-39D depict transduction efficiency and viral integrations per cell of CoStAR modified TILs. Nontransduced (Non-Td) and anti-FOLR1 CoStAR transduced (Td) TILs were assessed for the transduction efficiency on day 25 post REP in the CD3+(FIG. 39A), CD4+(FIG. 39B) and CD8+(FIG. 39C) cell populations. The viral copy number (VCN) was assesed to determine viral integrations (FIG. 39D). Shapes corresponding to the each of the 5 ovarian cancer samples are depicted. Filled shapes are non-Td and clear shapes are Td TILs.

FIG. 40 depicts CD4 and CD8 populations in post-REP TILs. Nontransduced (Non-Td) and anti-FOLR1 CoStAR− transduced (Td) and anti-FOLR1 CoStAR+Td TILs were assessed for the CD4 and CD8 composition on D25 post-REP using flow cytometry. Statistical analysis was performed using a two-way ANOVA with matched Tukey's multiple comparisons post-test.

FIGS. 41A-41C depict the effect of CoStAR modification on the differentiation status of TILs. Nontransduced (Non-Td), anti-FOLR1 CoStAR− transduced (Td) TILs and anti-FOLR1 CoStAR+Td TILs were assessed for their differentiation status on D25 post-REP from total T cell (FIG. 41A), CD4+(FIG. 41B) and CD8+(FIG. 41C) cell populations. Statistical analysis was performed using a two-way ANOVA with matched Tukey's multiple comparisons test. Statistical significance was observed for the Tscm population between the anti-FORL1 CoStAR− Td TILs and anti-FOLR1 CoStAR+Td TILs for CD3+(FIG. 41A) and CD4+(FIG. 41B) populations. *p<0.05.

FIGS. 42A-42B depict effects of CoStAR modification of TILs on co-inhibitory or co-stimulatory marker expression. Nontransduced (Non-Td), anti-FOLR1 CoStAR-transduced (Td) TILs and anti-FOLR1 CoStAR+Td TILs were assessed for the expression of co-inhibitory and co-stumulatory markers in CD4+(FIG. 42A) and CD8+(FIG. 42B) cell populations. Shapes corresponding to the each of the 5 ovarian cancer samples are depicted regardless of the filling. Black, dark grey and light grey bars represent Non-Td TILs, anti-FOLR1 CoStAR− Td and anti-FOLR1 CoStAR+Td TILs, respectively. Statistical analysis was performed using a Two-way ANOVA with a matched Tukey's multiple comparisons post-test. *p<0.05

FIGS. 43A-43B depicts effects of CoStAR modification on TCRαβ, TCRγδ and Treg frequency in TILs. Nontransduced (Non-Td), anti-FOLR1 CoStAR− transduced (Td) TILs and anti-FOLR1 CoStAR+Td TILs were assessed for the frequency of TCRαβ (CD3+TCRαβ+), and TCRγδ (CD3+TCRγ6+) in CD3+ cell populations (FIG. 43A). The frequency of Tregs in the CD4+ subpopulation was also assessed (FIG. 43B). Shapes corresponding to the each of the 5 ovarian cancer samples are depicted regardless of the filling. Black, dark grey and light grey bars represent Non-Td TILs, anti-FOLR1 CoStAR− Td TILs and anti-FOLR1 CoStAR+Td TILs, respectively. Statistical analysis was performed using a two-way ANOVA with matched Tukey's multiple comparisons post-test (FIG. 43A) and a one-way ANOVA with a Friedman's post-test (FIG. 43B).

FIGS. 44A-44C depict effects of CoStAR modification of TIL on cytokine production upon mitogenic activation. Nontransduced (Non-Td), anti-FOLR1 CoStAR-transduced (Td) TILs and anti-FOLR1 CoStAR+Td TILs were assessed for the producrtion of cytokines upon 4 hour stimulation with PMA (50 ng/mL)/Ionomycin (1 μg/mL) in CD3+(FIG. 44A), CD4+(FIG. 44B) and CD8+(FIG. 44C) cell populations. Shapes corresponding to the each of the 5 OC samples are depicted regardless of the filling. Black, dark grey and light grey bars represent Non-Td TILs, anti-FOLR1 CoStAR− Td TILs and anti-FOLR1 CoStAR+Td TILs, respectively. Statistical analysis was performed using a two-way ANOVA with matched Tukey's multiple comparisons post-test. * p<0.05, *** p<0.001.

FIG. 45 depicts Expression of FOLR1 on OC digest cells. Cryopreserved autologous tumor digest was thawed and analyzed by flow cytometry to determine the surface expression of FOLR1 of each donor. All 5 donors used in the study are shown. Abbreviations: OC, ovarian cancer, FOLR1, folate receptor alpha; FOLR1 PE, anti-FOLR1 antibody conjugated to PE; PE, phycoerythrin.

FIGS. 46A-46D depict effect of CoStAR modification on cytokine producing cells upon coculture with autologous tumors. Non-Td and anti-FOLR1 CoStAR Td TILs were cocultured with autologous tumor digests for 16 hours and intracellular flow cytometry was performed to evaluate the proportion of cells producing cytokines. The frequency of IL-2 positive (FIG. 46A) CD4 and (FIG. 46B) CD8 TILs as well as the frequency of TNFα positive (FIG. 46C) CD4 and (FIG. 46D) CD8 TILs were assessed. Results represent 5 biological replicates with 3 technical replicates each. Statistical analysis was performed using a two-way ANOVA with Tukey's multiple comparisons test. *P<0.05.

FIGS. 47A-47E depict the effect of CoStAR modification on cytokine secretion upon coculture with autologous tumor digests expressing FOLR1. Non-Td and anti-FOLR1 CoStAR transduced (Td) TILs were cocultured with autologous tumor digest for 24 hours, following which supernatants were collected and analyzed for cytokine secretion using an MSD immunoassay. The graphs represent measured concentrations of (FIG. 47A) IL-2, (FIG. 47B) TNFα, (FIG. 47C) IL-13 and (FIG. 47D) IFNγ in coculture supernatants. (FIG. 47E) Correlation between IFNγ concentration and FOLR1 expression by autologous digest as determined in FIG. 1. Results represent 5 biological replicates with 3 technical replicates each. Statistical analysis was performed using a two-way ANOVA with Sidak's multiple comparisons test. Correlation analysis was performed using a simple linear regression. *P<0.05, **P<0.01, ***P<0.001.

FIG. 48 depicts assessment of MHC-dependent CoStAR functionality in cocultures with autologous tumor digests. Non-Td and anti-FOLR1 CoStAR transduced (Td) TILs were cocultured with autologous tumor digests in the presence of MHC blocking reagents or isotype controls for 24 hours. The supernatants were then analyzed for IFNγ cytokine production using the MSD immunoassay. Results represent 3 biological replicates with 3 technical replicates each. Statistical analysis was performed using a two-way ANOVA with Tukey's multiple comparisons test.

FIGS. 49A-49B depicts the effect of CoStAR modification in cytokine producing cell frequencies upon coculture with engineered cell lines. Non-Td and anti-FORL1 CoStAR transduced (Td) TILs were cocultured with engineered target cell lines for 16 hours and cytokine producing cells were measured using intracellular flow cytometry. Frequency of IL-2 positive (FIG. 49A) in CD4+(top) and CD8+(bottom) TILs following coculture with K-562 (left) and OVCAR-3 (right) derived lines. Frequency of TNFα positive (FIG. 49B) in CD4+(top) and CD8+(bottom) TILs following coculture with K-562 (left) and OVCAR-3 (right) derived lines. Results represent 5 biological replicates with 3 technical replicates each. Statistical analysis was performed using a two-way ANOVA with Tukey's multiple comparisons test. *P<0.05, **P<0.01, ***P<0.001, ****P<0.0001.

FIGS. 50A-50D depict the effect of CoStAR modification in cytokine secretion upon coculture with engineered cell lines. Non-Td and anti-FOLR1 CoStAR transduced (Td) TILs were cocultured with engineered target cell lines for 24 hours and MSD immunoassay was performed to evaluate the concentration of cytokines secreted. Cytokine concentrations for (FIG. 50A) IL-2, (FIG. 50B) TNFα, (FIG. 50C) IL-13 and (FIG. 50D) IFNγ following cocultures with (left) K-562, and (right) BA/F3 derived cell lines are shown. The results represent 5 biological replicates with 3 technical replicates each. Statistical analysis was performed using a two-way ANOVA with Tukey's multiple comparisons test. *P<0.05, **P<0.01, ***P<0.001, ****P<0.0001.

FIGS. 51A-51C depict assessment of the impact of CoStAR modification on the cytotoxic capacity of TILs against OKT3 bearing targets. Non-Td and anti-FOLR1 CoStAR transduced (Td) TILs were cocultured with BA/F3 or OVCAR-3 engineered cell lines and the cytotoxicity was assessed using a flow cytometry based (FIG. 51A) and an xCELLigence-based assay (FIGS. 51B and 51C), respectively. (FIG. 51B) E:T ratio of 1:5 and (FIG. 51C) E:T ratio of 1:30. Results represent 5 biological replicates with 3 technical replicates each. Statistical analysis was performed using a two-way ANOVA with Tukey's multiple comparisons test for flow cytometry data and 1-way ANOVA with Tukey's multiple comparisons test for xCELLigence data. *P<0.05, **P<0.01.

FIGS. 52A-52D depict expansion of NSCLC TILs with >80% viability (FIG. 52A) and 100-200 million cells (FIG. 52B), 40-55% CoStar transduction in CD3+ T cells (FIG. 52C) and >90% CD3+ T cell purity (FIG. 52D).

FIG. 53 depicts expansion of NSCLC TILs with >80% viability. There was no difference in CoStAR transduced and nontransduced populations in terms of viability and cell expansion.

FIG. 54 depicts expansion of NSCLC TILs with 50-500 million cells at harvest. There was no differnce in CoStAR transduced and nontransduced populations in terms of viability and cell expansion.

FIG. 55 depicts T cell populations of seven patient samples.

FIG. 56 depicts CD3 vs CD56 which marks CD3+ as T cells and CD3-CD56+ as NK cells.

FIG. 57 depicts quantification of non-transduced and CoStAR transduced T cells.

FIG. 58 depicts CoStAR staining on T cell subsets.

FIGS. 59A-59D depict significant increase in viability of ovarian cancer TILs during REP and >90% viability at the end of the manufacturing process, with 100-300 million cells at harvest. FIG. 59A depicts the cell count and FIG. 59B depicts the viability of ovarian cancer TILs over 10 days. FIG. 59C depicts the cell count and FIG. 59D depicts the viability following the manufacturing process.

FIGS. 60A-60D depicts renal cancer TILs demonstrated consistently >50% viability during outgrowth and >90% viability at the end of the manufacturing process, with 100-350 million cells at harvest. FIG. 60A depicts the cell count and FIG. 60B depicts the viability of renal cancer TILs over 10 days. FIG. 60C depicts the cell count and FIG. 60D depicts the viability following the manufacturing process.

FIG. 61 depicts an experimental setup as outlined in Example 16, wherein CD40 signaling motif mutant constructs were incorporated into a cell to screen for changes to transcription.

FIG. 62A depicts the 23 day experimental approach for manufacturing TRAF variant CoSTARs in T cells, as outlined in Example 17.

FIG. 62B depicts the 26 day experimental approach for monitoring the effect of TRAF variant CoSTARs on proliferation in T cells, as outlined in Example 17.

FIG. 63A-63C depicts the change in total number of CD2+ T cells expressing TRAF variant CoSTARs with IL-2 treatment. Graphs are given for change in cell numbers from cells collected from donor HD 702C (FIG. 63A), HD 1004 (FIG. 63B), and HD 2000 (FIG. 63C).

FIG. 64A-64C depicts the fold-change in number of CD2+ T cells expressing TRAF variant CoSTARs with IL-2 treatment. Graphs are given for the fold change in cell numbers from cells collected from donor HD 702C (FIG. 64A), HD 1004 (FIG. 64B), and HD 2000 (FIG. 64C).

FIG. 65A-65C depicts the change in total number of CD2+ T cells expressing TRAF variant CoSTARs, wherein the T cells did not receive any IL-2 treatment. Graphs are given for change in cell numbers from cells collected from donor HD 702C (FIG. 65A), HD 1004 (FIG. 65B), and HD 2000 (FIG. 65C).

FIG. 66A-66C depicts the fold-change in number of CD2+ T cells expressing TRAF variant CoSTARs, wherein the T cells did not receive any IL-2 treatment. Graphs are given for the fold change in cell numbers from cells collected from donor HD 702C (FIG. 66A), HD 1004 (FIG. 66B), and HD 2000 (FIG. 66C).

FIG. 67 depicts some embodiments of CD40 TRAF variants of SEQ ID NOS: 510-519. In this figure, the underlined region is CD40, sequences highlighted in grey is the TRAF6 binding domain region, sequences highlighted in light grey is the TRAF2/3 binding domain, and sequences highlighted in grey and surrounded with double brackets is the TRAF2 binding domain.

FIG. 68 depicts the transduction levels of constructs into primary T cells collected from donor HD 702C, HD 1004, and HD 2000.

FIGS. 69A-69F depict the fold change in cell proliferation compared to baseline in primary human donors 702C (FIGS. 69A and 69D) 1004 (FIGS. 69B and 69E), and 2000 (FIGS. 69C and 69F) primary T cells expressing CoStAr constructs with TRAF6 variants, and with either IL-2 treatment (FIGS. 69A-69C) or without IL-2 treatment (FIGS. 69D-69F), as outlined in Example 18.

FIGS. 70A-70F depict the fold change in cell proliferation compared to baseline in primary human donors 702C (FIGS. 70A and 70D) 1004 (FIGS. 70B and 70E), and 2000 (FIGS. 70C and 70F) primary T cells expressing CoStAr constructs with TRAF2/3 variants, and with either IL-2 treatment (FIGS. 70A-70C) or without IL-2 treatment (FIGS. 70D-70F), as outlined in Example 18.

FIGS. 71A-71F depict the fold change in cell proliferation compared to baseline in primary human donors 702C (FIGS. 71A and 71D) 1004 (FIGS. 71B and 71E), and 2000 (FIGS. 71C and 71F) primary T cells expressing CoStAr constructs with TRAF2 variants, and with either IL-2 treatment (FIGS. 71A-71C) or without IL-2 treatment (FIGS. 71D-71F), as outlined in Example 18.

FIGS. 72A-72F depict the fold change in cell proliferation compared to baseline in primary human donors 702C (FIGS. 72A and 72D) 1004 (FIGS. 72B and 72E), and 2000 (FIGS. 72C and 72F) primary T cells expressing CoStAr constructs with various TRAF variants, and with either IL-2 treatment (FIGS. 72A-72C) or without IL-2 treatment (FIGS. 72D-72F), as outlined in Example 18.

FIGS. 73A-73D depict the combined data of FIGS. 69A-69C, 70A-70C, 71A-71C, and 72A-72C, in which the fold change in cell proliferation compared to baseline in primary T cells across three donors is graphed over time. The graphs show cells treated with IL-2, and represent the combined data for cells expressing a TRAF6 (FIG. 73A), TRAF2/3 (FIG. 73B), TRAF2 (FIG. 73C), or other TRAF (FIG. 73D) variant.

FIGS. 74A-74D depict the combined data of FIGS. 69A-69C, 70A-70C, 71A-71C, and 72A-72C, in which the fold change in cell proliferation compared to baseline in primary T cells across three donors is graphed over time. The graphs show cells that were not given IL-2 treatment, and represent the combined data for cells expressing a TRAF6 (FIG. 74A), TRAF2/3 (FIG. 74B), TRAF2 (FIG. 74C), or other TRAF (FIG. 74D) variant.

FIGS. 75A-75B depict the combined fold changes in cell proliferation compared to baseline in primary human T cells from 3 donors at Day 21 for cells with IL-2 treatment (FIG. 75A) and without IL-2 treatment (FIG. 75B).

FIGS. 76A-76C depict non-limiting examples of schematic models for universal costimulatory proteins with TRAF variants. (FIG. 76A) TCR incorporated antigen agnostic receptor (TIAAR) comprises modifying components of the TCR complex and associated signaling adaptors. (FIG. 76B) A constitutive costimulatory receptor comprising transmembrane domains (TMDs) and features that enable inducible or constitutive activation. (FIG. 76C) An inducible costimulatory receptor capable of induction and activation by extracellular ligand binding. In this non-limiting example, the TRAF variant is part of the CD40 domain (shown as “CD40*”).

FIGS. 77A-77C depict a non-limiting example protein construct of some embodiments herein. FIG. 77A is a general schematic of the protein comprising a universal CoStAR sequence, further comprising an optional section, a binding domain, a CD28 domain, and a CD40 domain with a TRAF variant. FIG. 77B is a schematic of some embodiments provided herein. FIG. 77C outlines a set of sequences of some embodiments provided herein.

FIG. 78 depicts set of sequences of some embodiments provided herein (SEQ ID NOS: 40839-41074). In some embodiments, these sequences are excluded from the consensus sequences or other TRAF variant constructs provided herein (including excluded from those in FIGS. 84 and 85).

FIG. 79 depicts set of sequences of some embodiments provided herein (SEQ ID NOS: 41075-43474). In some embodiments, these sequences are excluded from the consensus sequences or other TRAF variant constructs provided herein (including excluded from those in FIGS. 84 and 85).

FIG. 80 depicts a non-limiting example of mutation analysis of CD40 using constructs CTP195, CTP196, and CTP197 to assess the function of signaling motifs, as described in Example 13.

FIG. 81 depicts a non-limiting example of mutation analysis of CD40 using constructs CTP195, CTP196, CTP197, CTP198, and CTP199 to assess the function of signaling motifs, as described in Example 13.

FIG. 82A depicts the minor consensus sequence generated from the alignments of variants in the TRAF2 domain as shown in Table 1, wherein the highlighted amino acids are conserved across variants.

FIG. 82B depicts the major consensus sequence generated from the alignments of variants in the TRAF2 domain as shown in Table 1, wherein the highlighted amino acids are conserved across variants.

FIG. 82C depicts the consensus sequence generated from the alignments of variants in the TRAF6 domain as shown in Table 3, wherein the highlighted amino acids are conserved across variants.

FIG. 83 depicts non-limiting example sequences of variants in the TRAF2 domain (SEQ ID NOS: 520-679).

FIG. 84 depicts non-limiting example sequences of variants in the TRAF2/3 domain (SEQ ID NOS: 680-839).

FIG. 85 depicts non-limiting example sequences of variants in the TRAF6 domain (SEQ ID NOS: 840-40839).

FIGS. 86A-86D illustrate a schematic showing some embodiments of a FOLR1αCoStAR and/or a fusion protein with a CD40 variant. Some general embodiments are depicted in FIG. 86A. FIG. 86B depicts some embodiments of a FOLR1a CoStAR or a fusion protein. FIG. 86C depicts some embodiments of a CoStAR or a fusion protein. FIG. 86D depicts some embodiments of a CoStAR or a fusion protein. In some embodiments, the sequences described the structure in its entirety and no further functional aspects are required to describe the CoStAR or fusion protein. In some embodiments, any of the CD40, TRAF2, TRAF2/3, or TRAF6 variants provided herein, including those in FIGS. 28, 67, 77A-77C, 83-89D, and 92 (excluding those in FIGS. 78 and 79), and including those in Tables 2, 4-5, and 7-14, and including SEQ ID NOS: 42, 475-479, 494-498, 505, 510-519, and 840-40839, and Consensus sequences A-C, can be used in the present method and/or construct and/or protein.

FIGS. 87A-87D illustrate a schematic showing some embodiments of an anti-pembrolizumab CoStAR and/or a fusion protein with a CD40 variant. Some general embodiments are depicted in FIG. 87A. FIG. 87B depicts some embodiments of an anti-pembrolizumab CoStAR or a fusion protein. FIG. 87C depicts some embodiments of a CoStAR or a fusion protein. FIG. 87D depicts some embodiments of a CoStAR or a fusion protein. In some embodiments, the sequences describe the structure in its entirety and no further functional aspects are required to describe the CoStAR or fusion protein. In some embodiments, any of the CD40, TRAF2, TRAF2/3, or TRAF6 variants provided herein, including those in FIGS. 28, 67, 77A-77C, 83-89D, and 92 (excluding those in FIGS. 78 and 79), and including those in Tables 2, 4-5, and 7-14, and including SEQ ID NOS: 42, 475-479, 494-498, 505, 510-519, and 840-40839, and Consensus sequences A-C, can be used in the present method and/or construct and/or protein.

FIGS. 88A-88D illustrate a schematic showing some embodiments of a CEA CoStAR and/or a fusion protein with a CD40 variant. Some general embodiments are depicted in FIG. 88A. FIG. 88B depicts some embodiments of a CEA CoStAR or a fusion protein. FIG. 88C depicts some embodiments of a CoStAR or a fusion protein. FIG. 88D depicts some embodiments of a CoStAR or a fusion protein. In some embodiments, the sequences describe the structure in its entirety and no further functional aspects are required to describe the CoStAR or fusion protein. In some embodiments, any of the CD40, TRAF2, TRAF2/3, or TRAF6 variants provided herein, including those in FIGS. 28, 67, 77A-77C, 83-89D, and 92 (excluding those in FIGS. 78 and 79), and including those in Tables 2, 4-5, and 7-14, and including SEQ ID NOS: 42, 475-479, 494-498, 505, 510-519, and 840-40839, and Consensus sequences A-C, can be used in the present method and/or construct and/or protein.

FIGS. 89A-89D illustrate a schematic showing some embodiments of a CEA CoStAR and/or a fusion protein with a CD40 variant. Some general embodiments are depicted in FIG. 89A. FIG. 89B depicts some embodiments of a MSLN CoStAR or a fusion protein. FIG. 89C depicts some embodiments of a CoStAR or a fusion protein. FIG. 89D depicts some embodiments of a CoStAR or a fusion protein. In some embodiments, the sequences describe the structure in its entirety and no further functional aspects are required to describe the CoStAR or fusion protein. In some embodiments, any of the CD40, TRAF2, TRAF2/3, or TRAF6 variants provided herein, including those in FIGS. 28, 67, 77A-77C, 83-89D, and 92 (excluding those in FIGS. 78 and 79), and including those in Tables 2, 4-5, and 7-14, and including SEQ ID NOS: 42, 475-479, 494-498, 505, 510-519, and 840-40839, and Consensus sequences A-C, can be used in the present method and/or construct and/or protein.

FIGS. 90A-90C depict the quantity of cytokines TNF-alpha (FIG. 90A), IFN-gamma (FIG. 90B), and IL-2 (FIG. 90C) present after expression of T cells with CD40 variants, as outlined in Example 19.

FIGS. 91A-91B depict the IL-2 production (FIG. 91A) and fold expansion (FIG. 91B) in cells expressing one of constructs CTP194-CTP200, as outlined in Example 20.

FIG. 92 depicts schematics of the protein constructs CTP188-CTP202, as described herein.

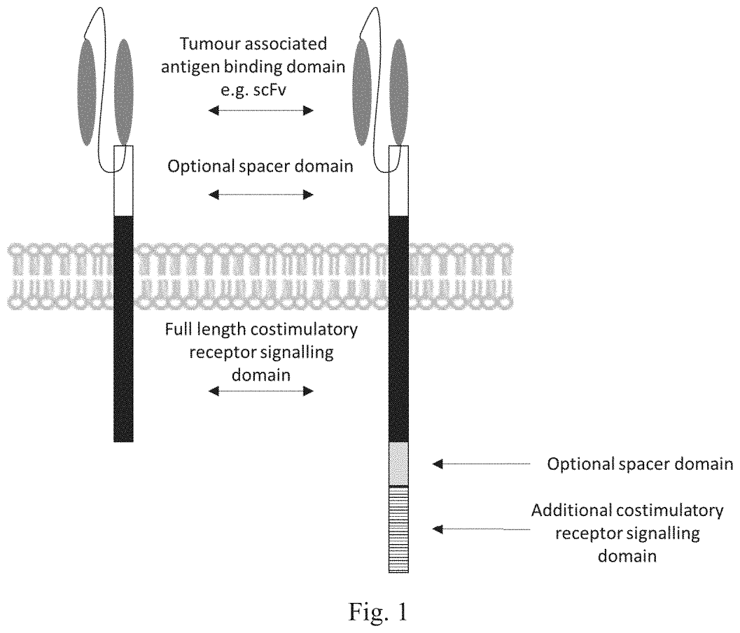

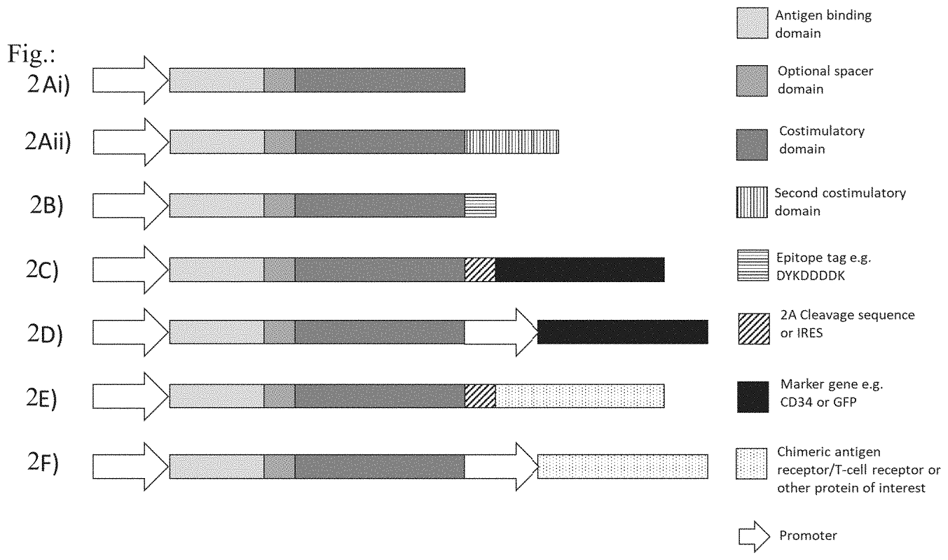

### DETAILED DESCRIPTION OF THE INVENTION

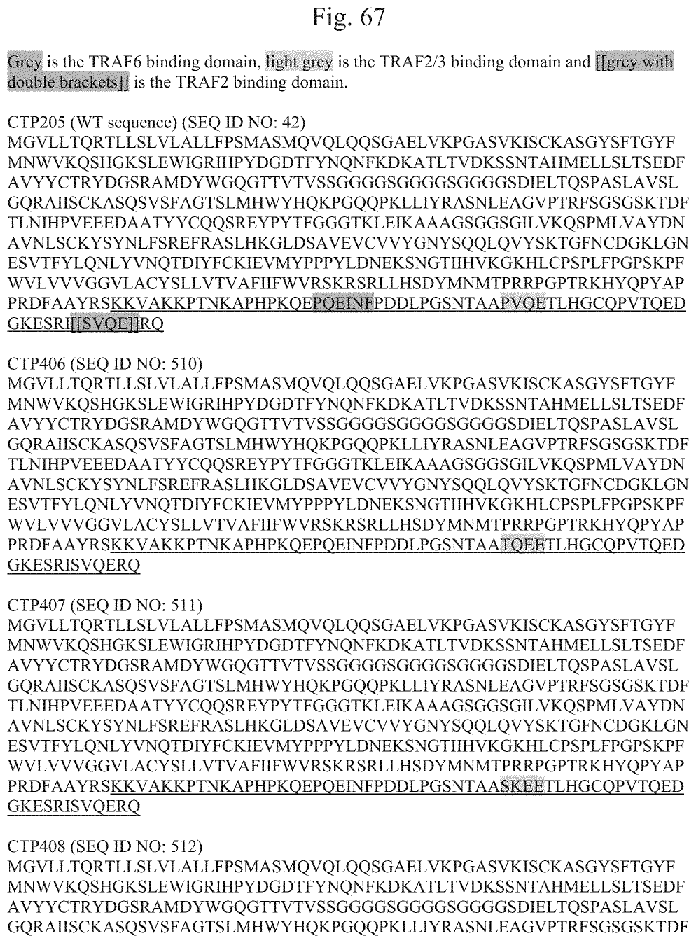

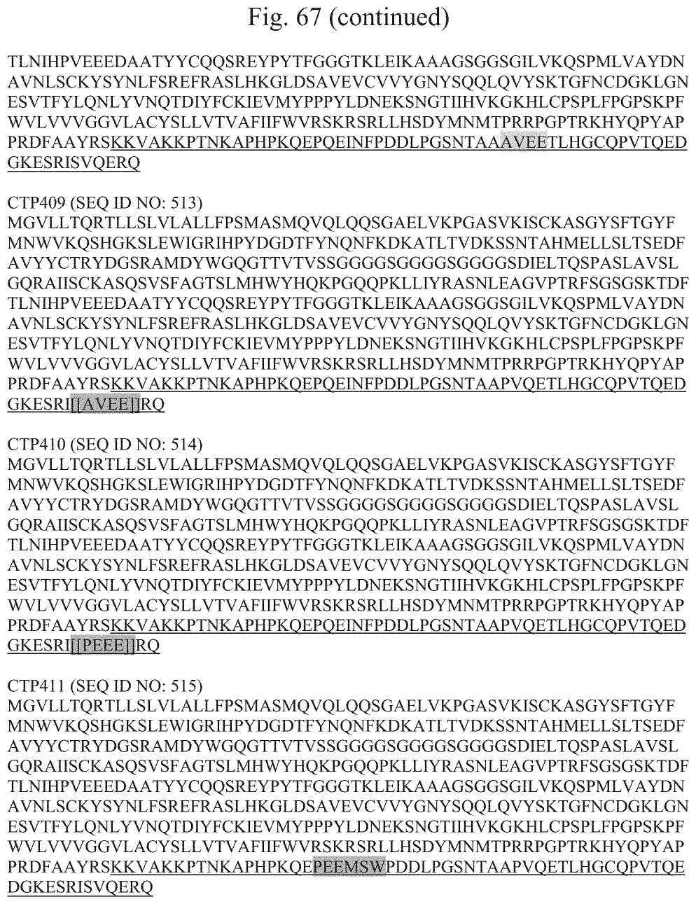

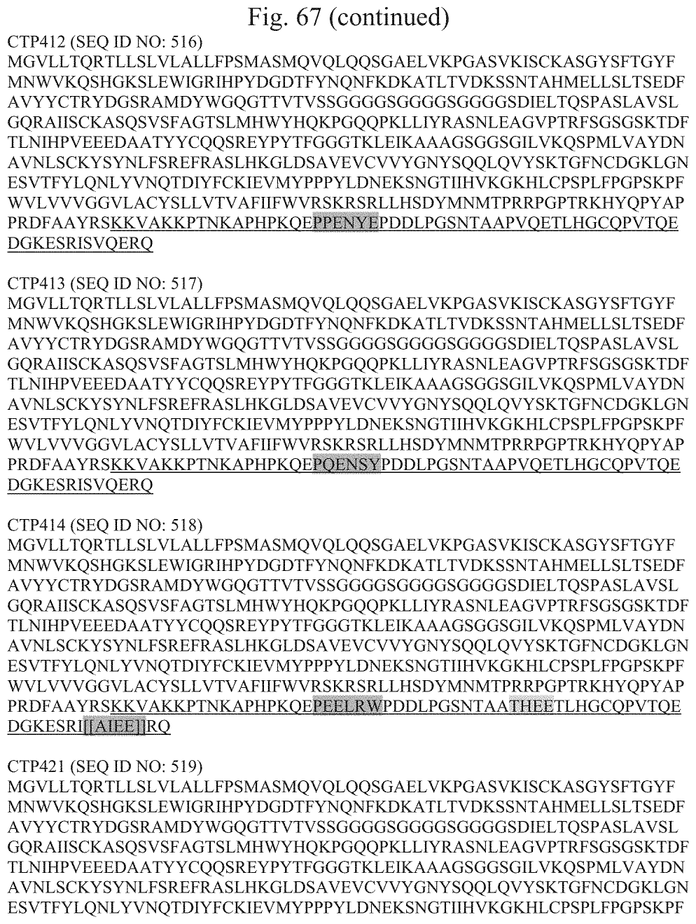

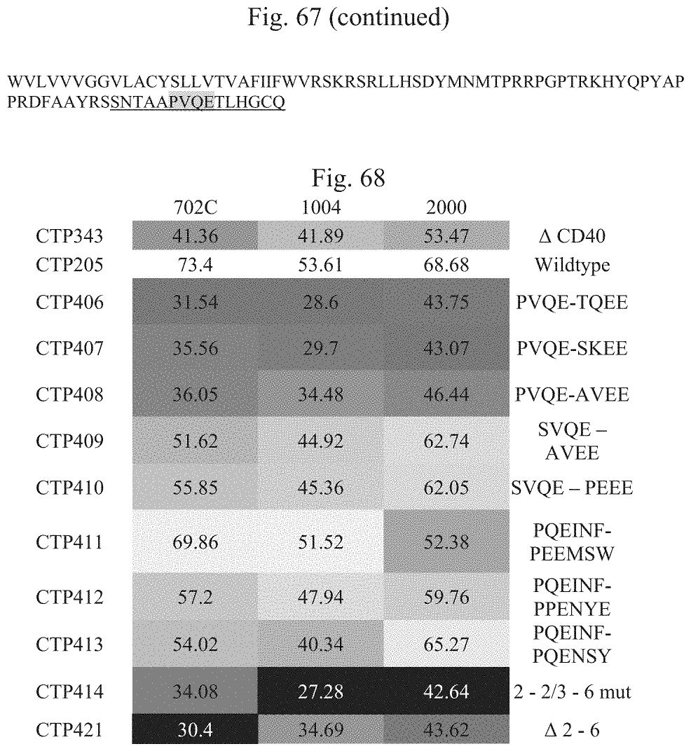

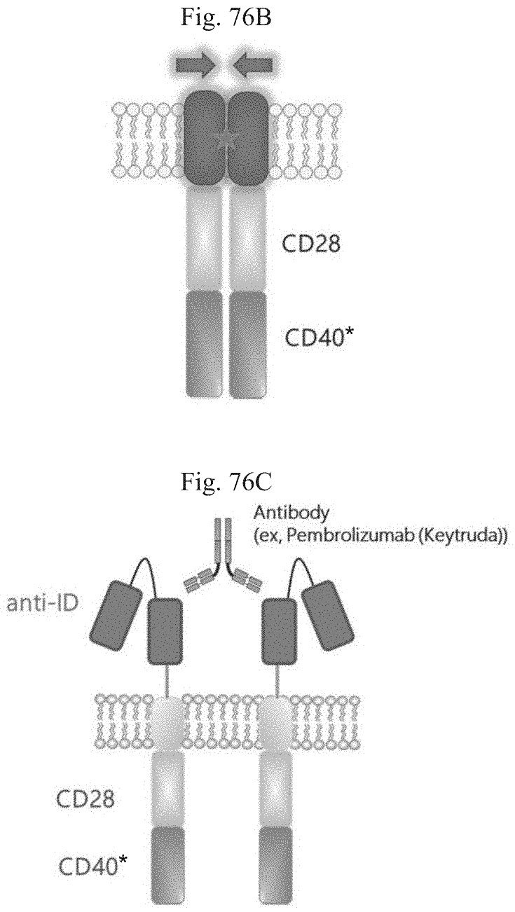

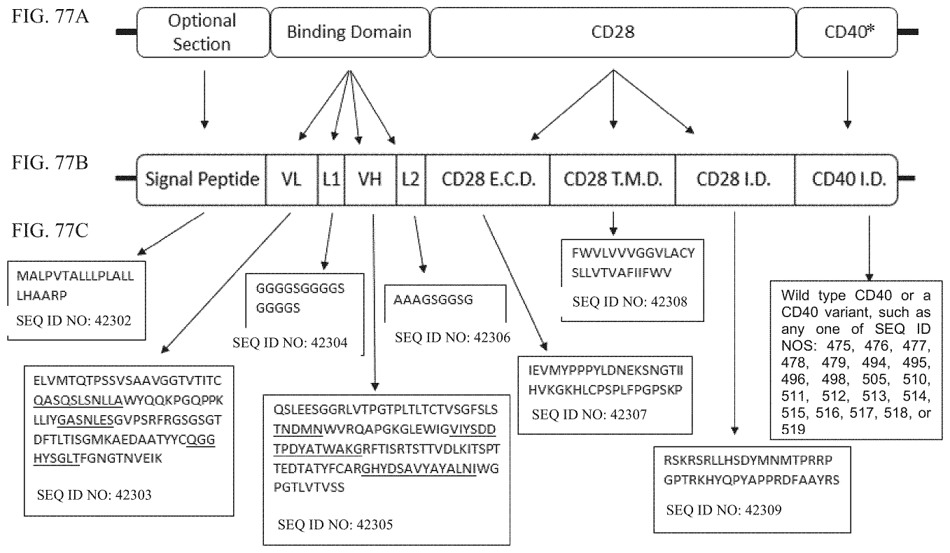

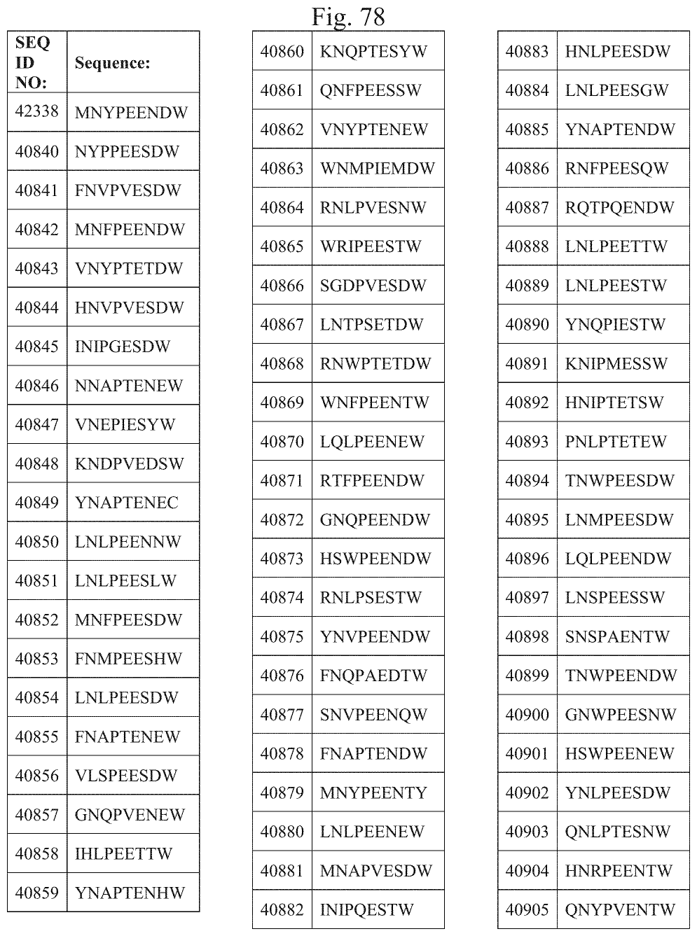

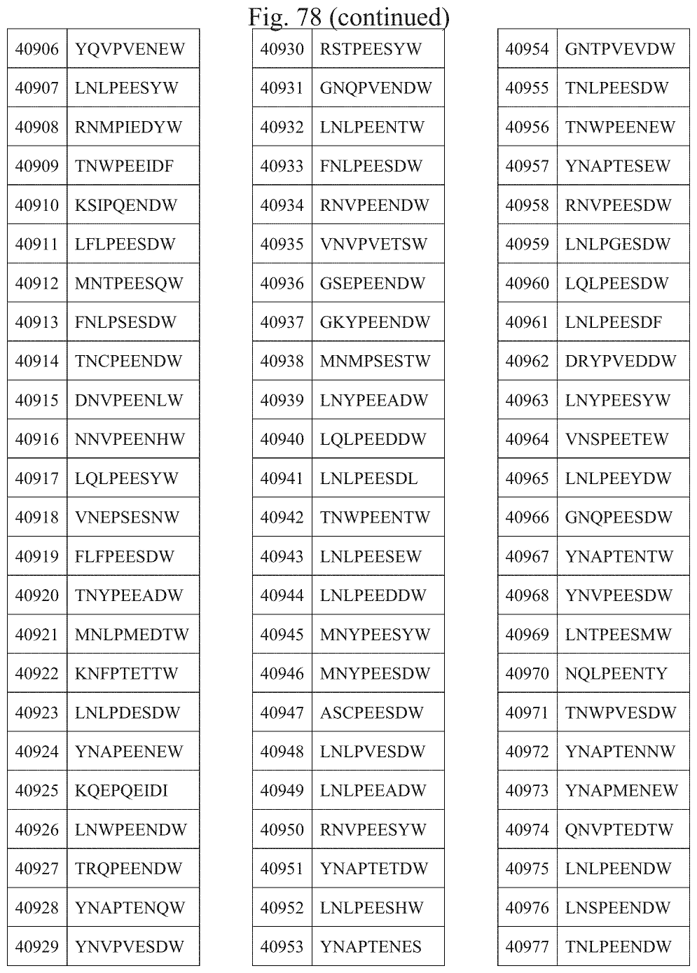

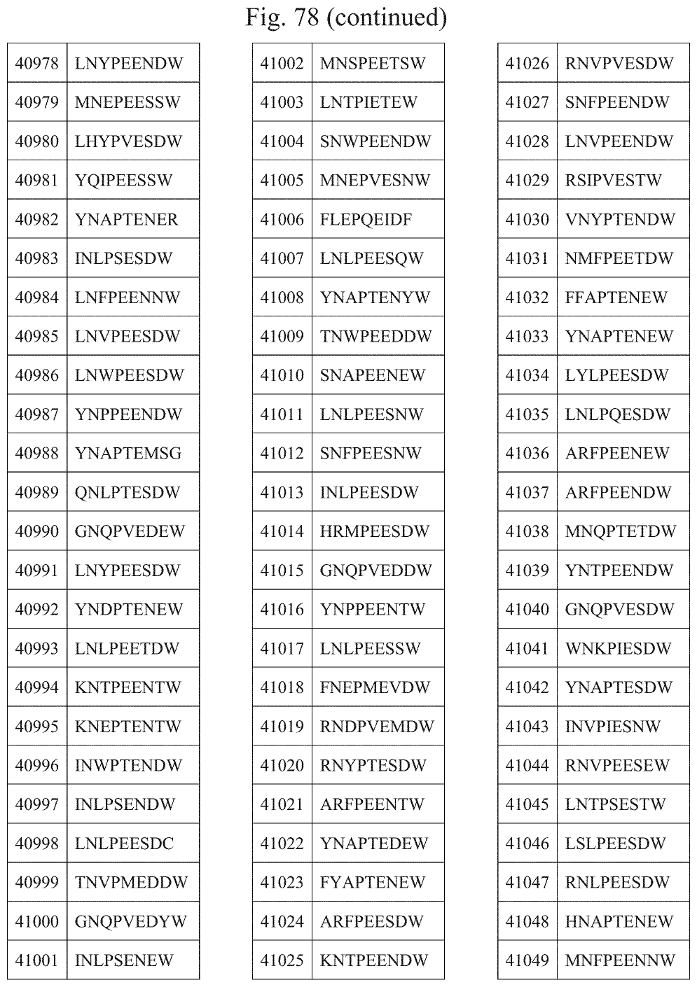

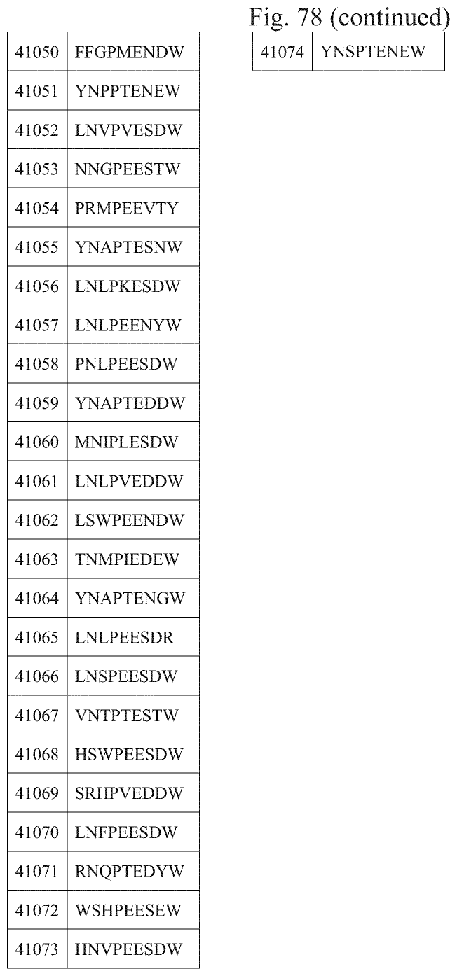

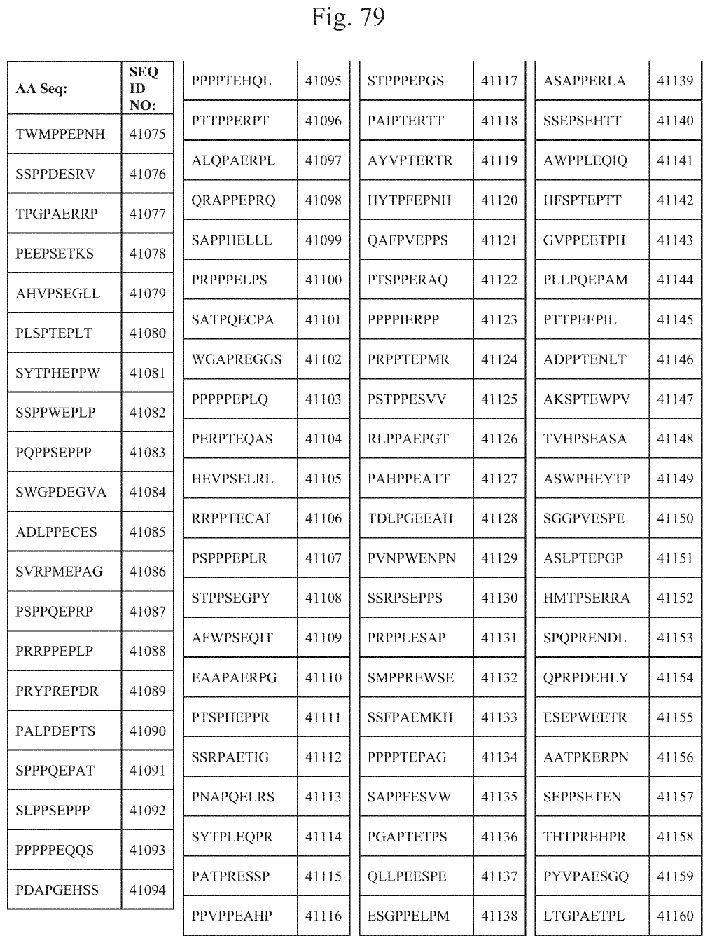

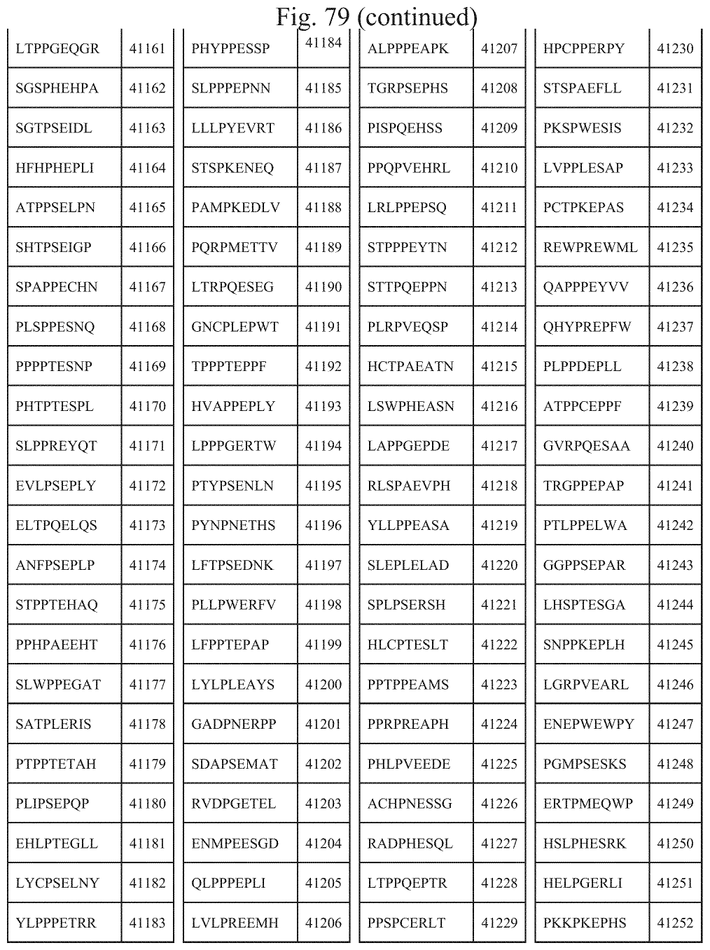

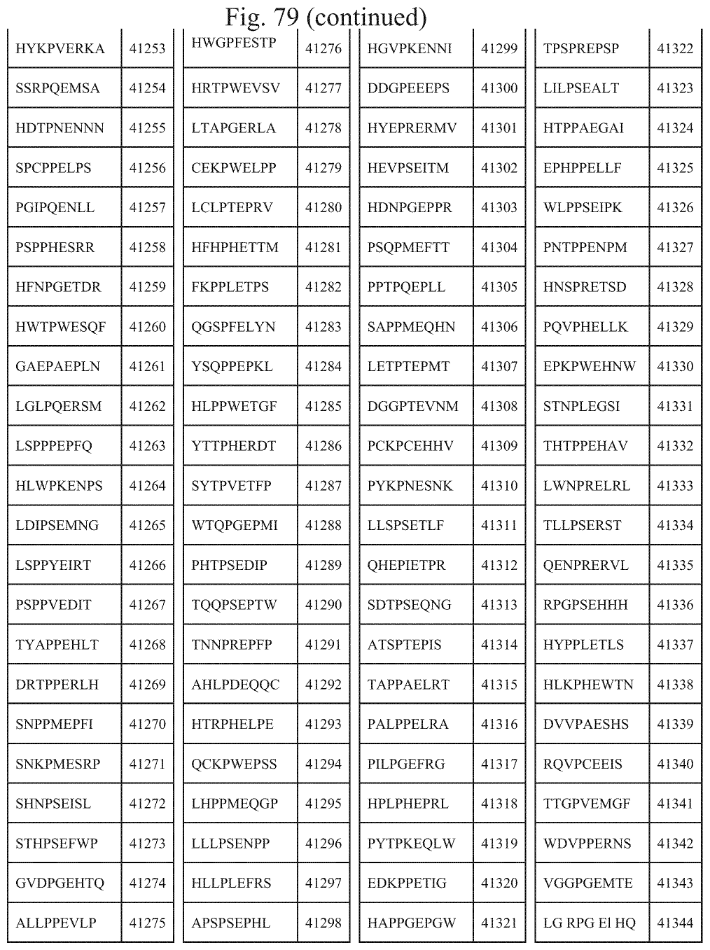

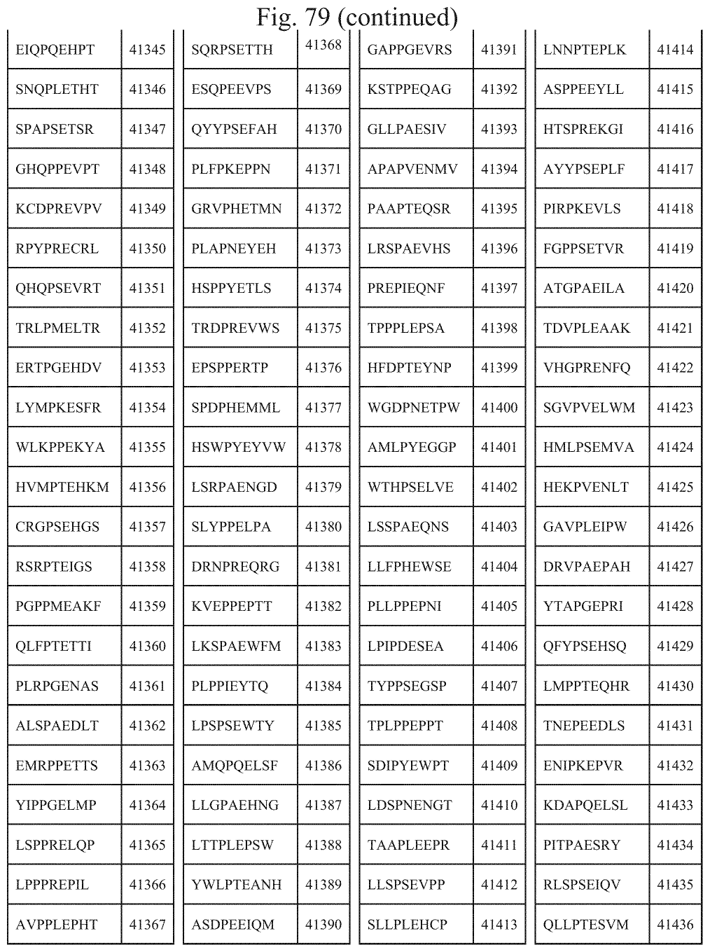

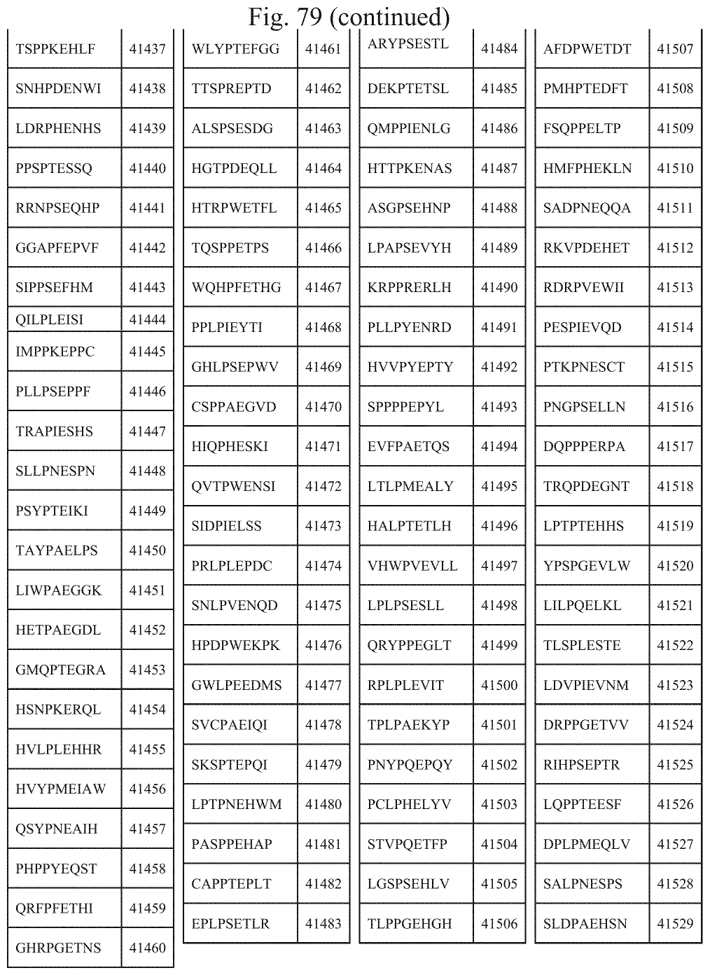

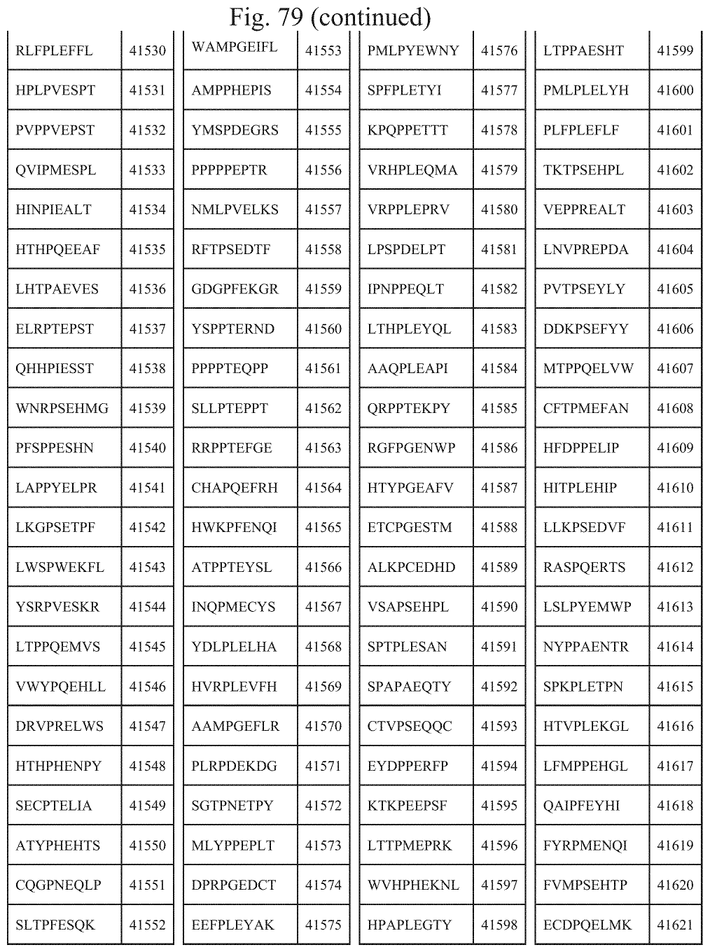

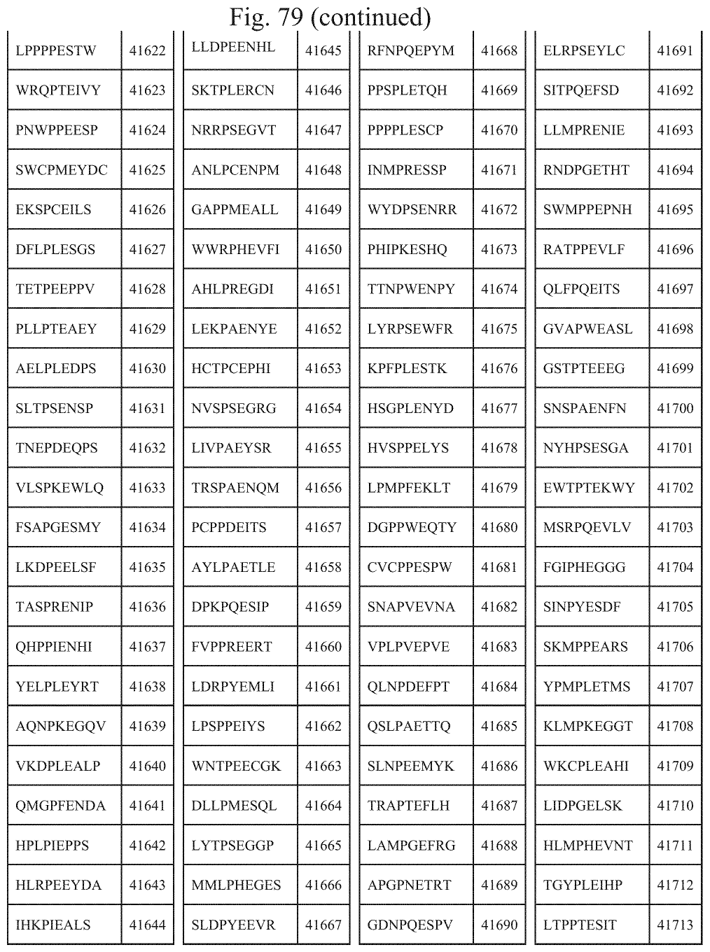

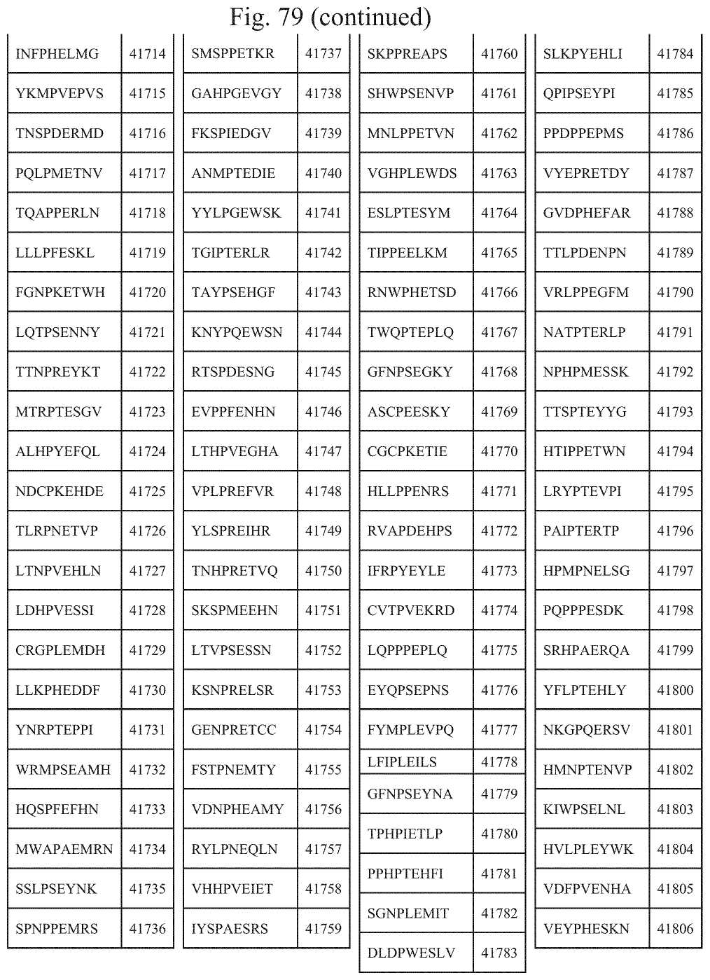

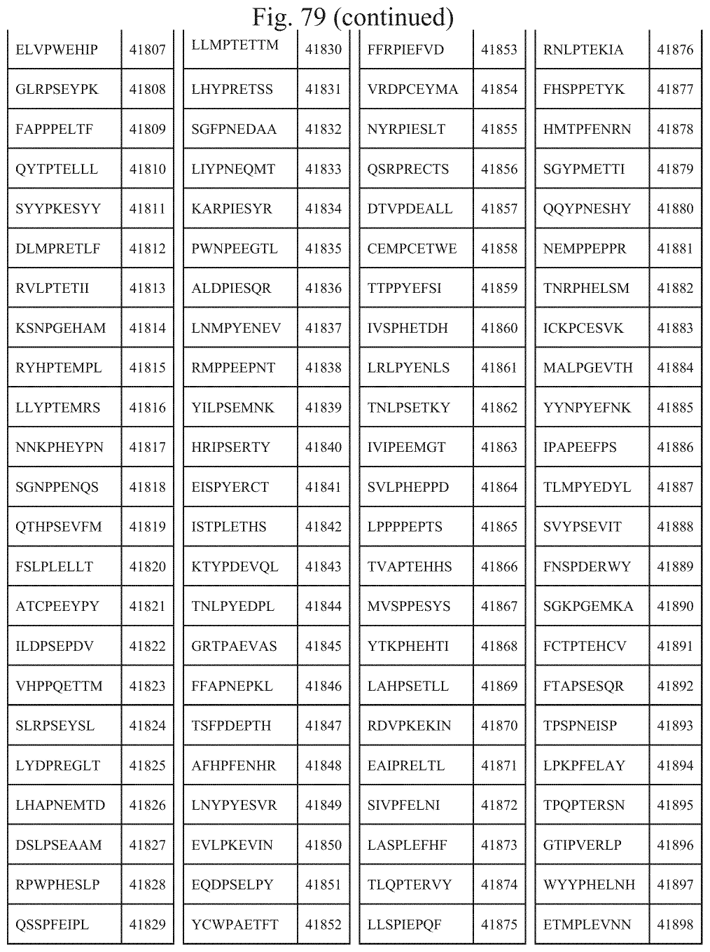

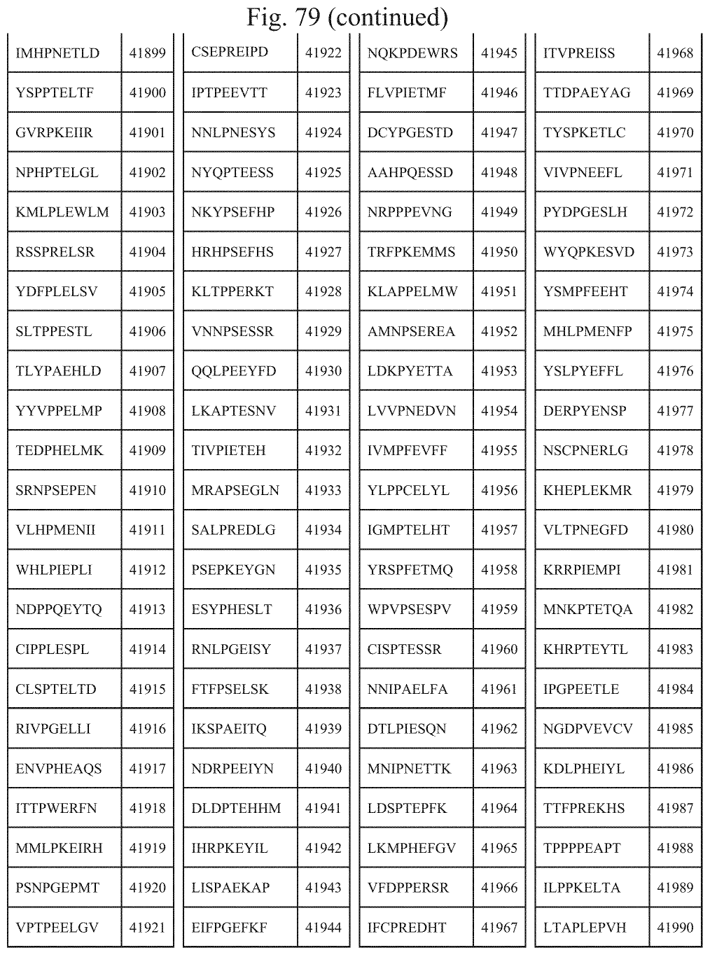

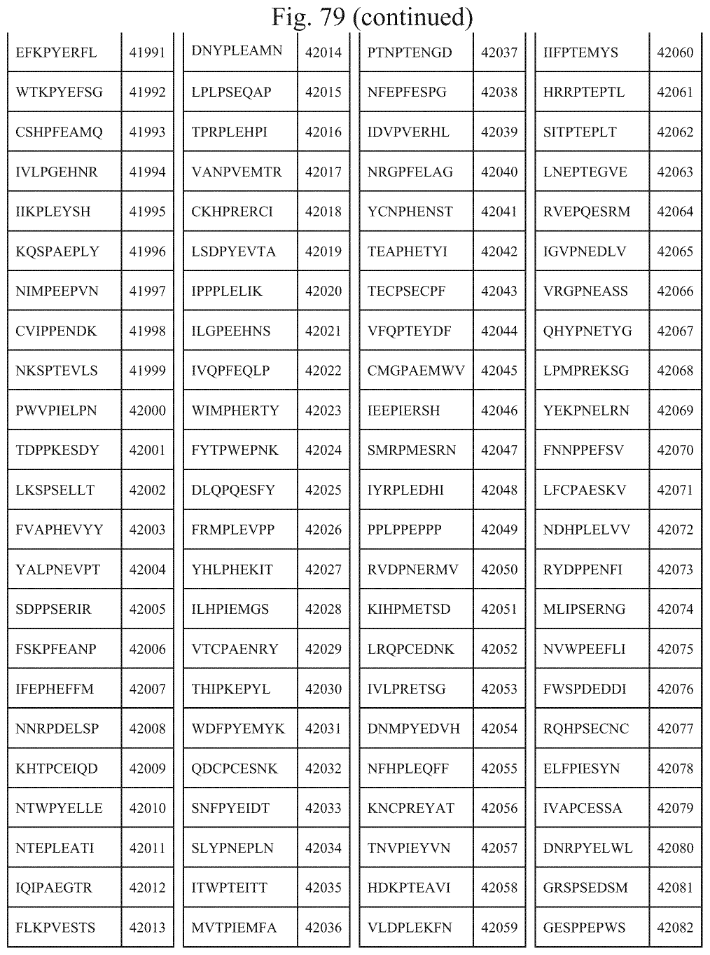

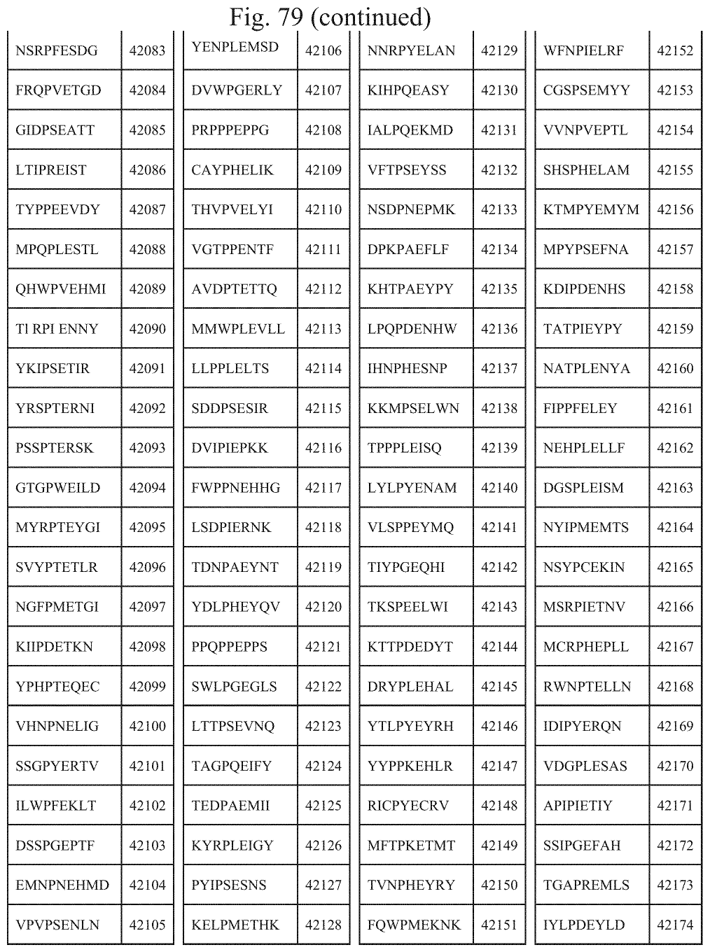

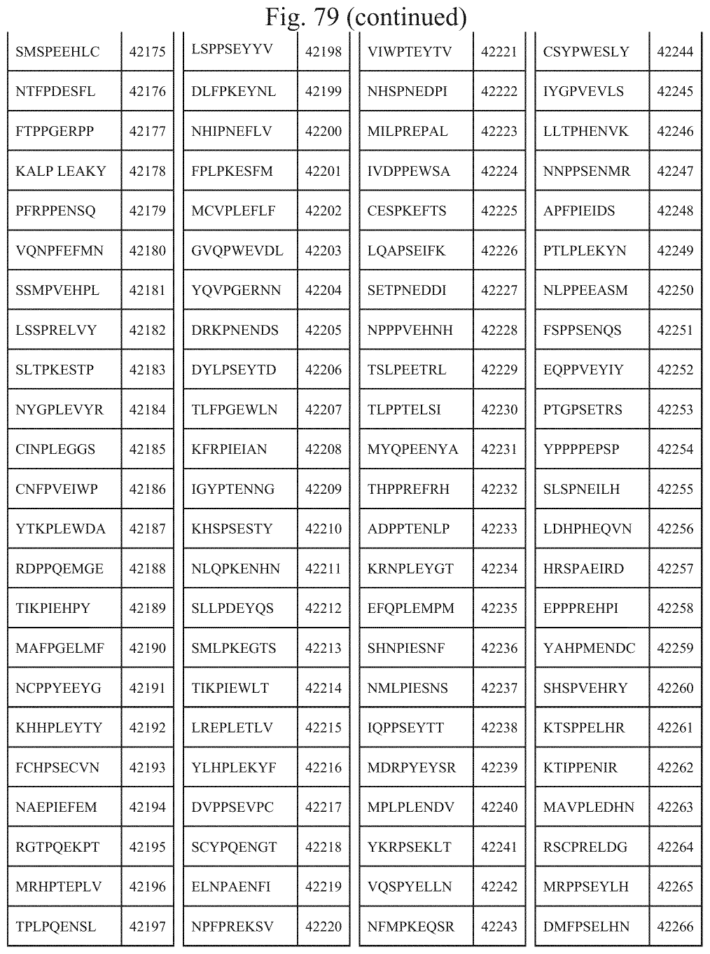

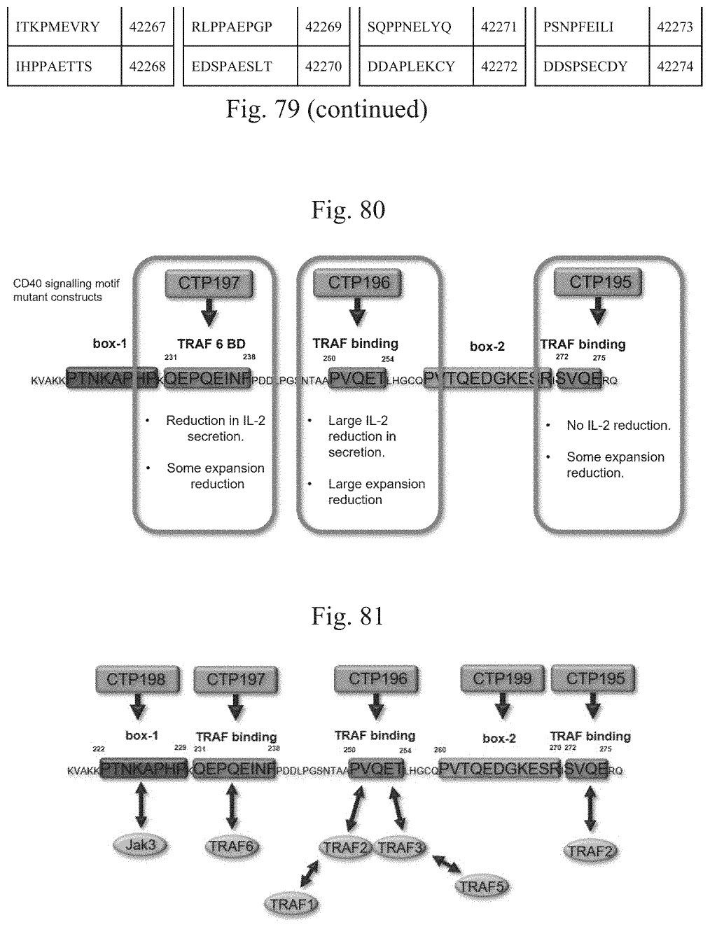

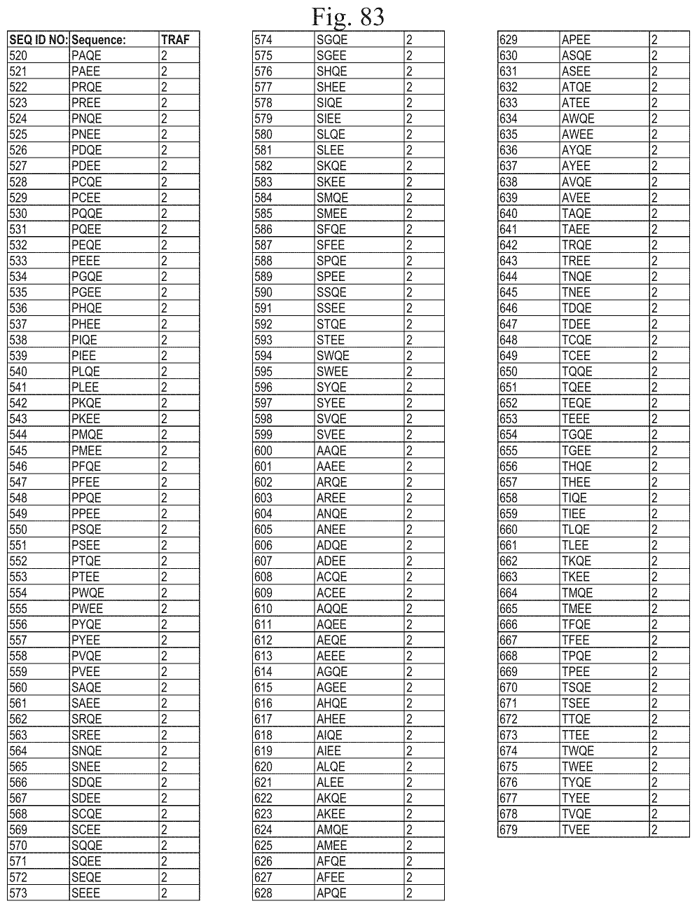

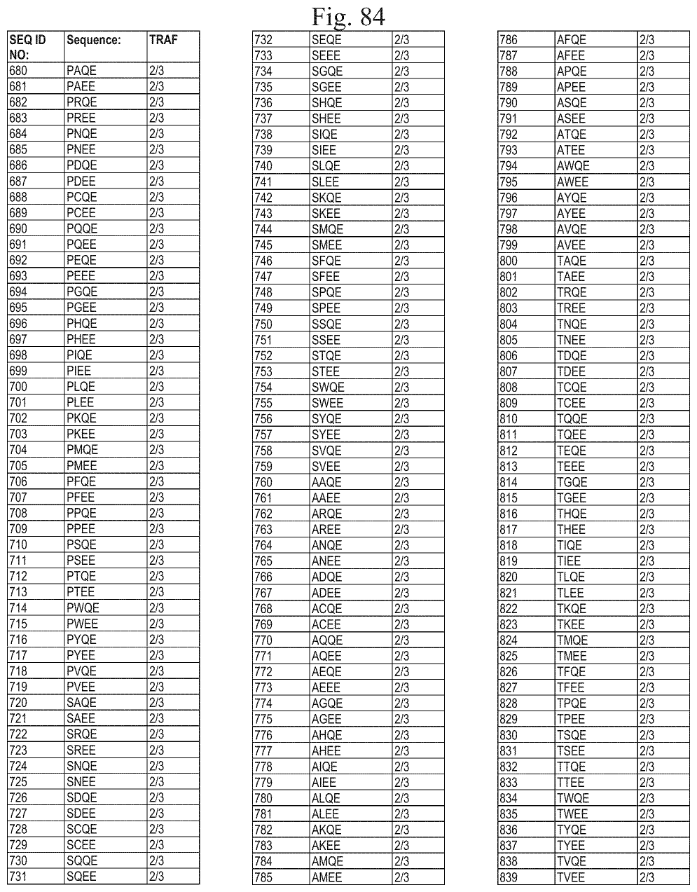

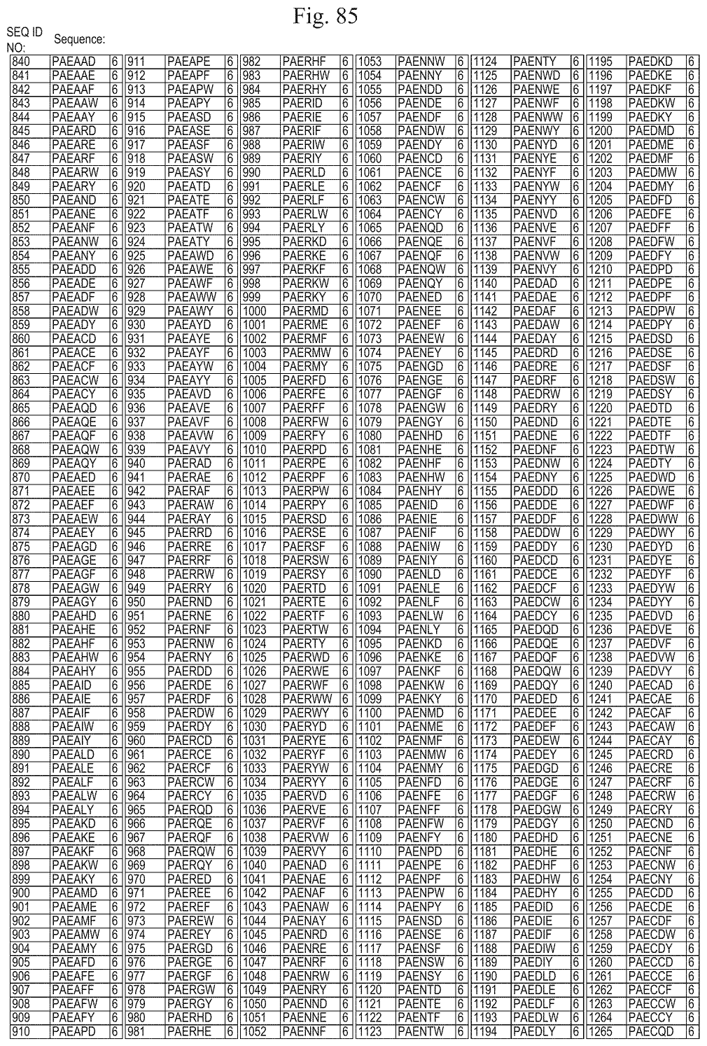

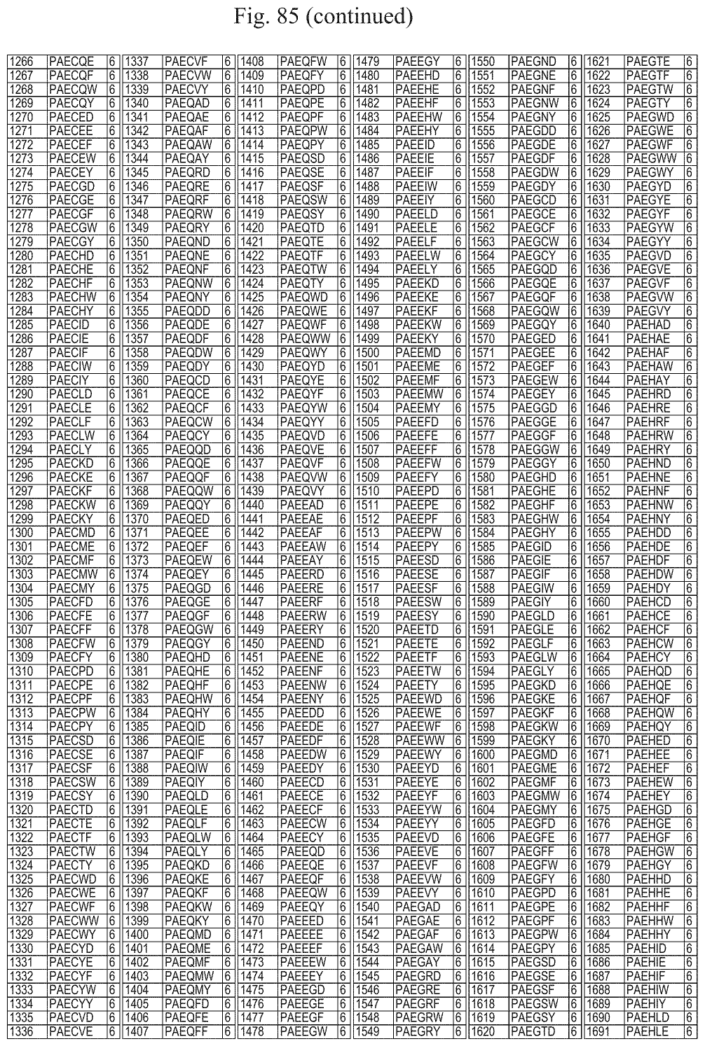

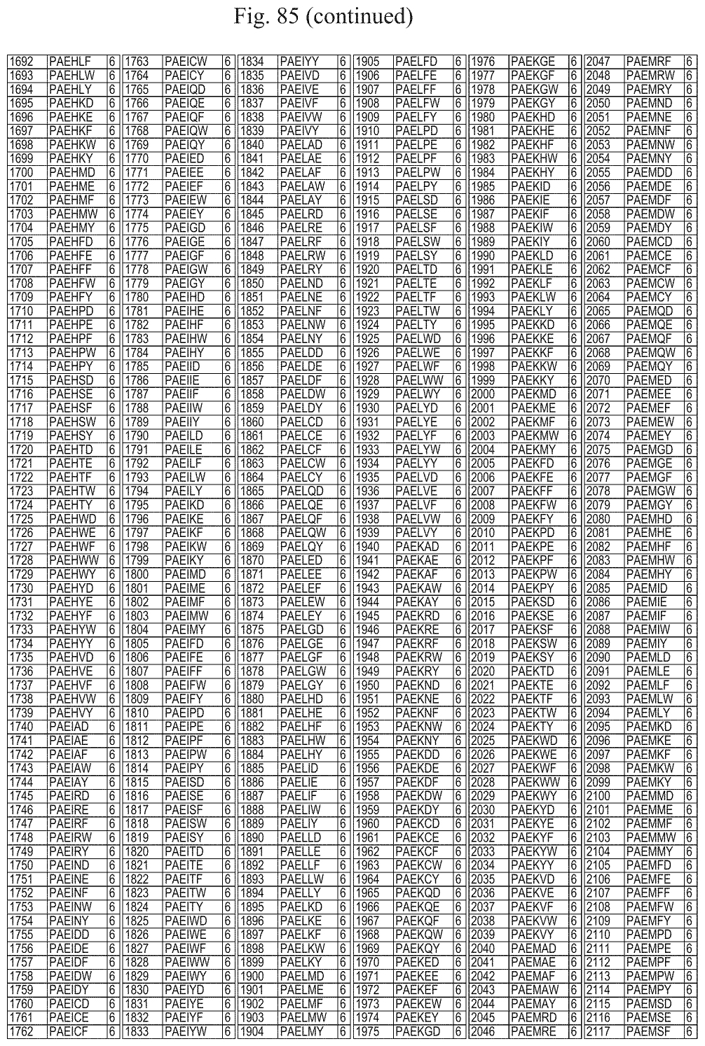

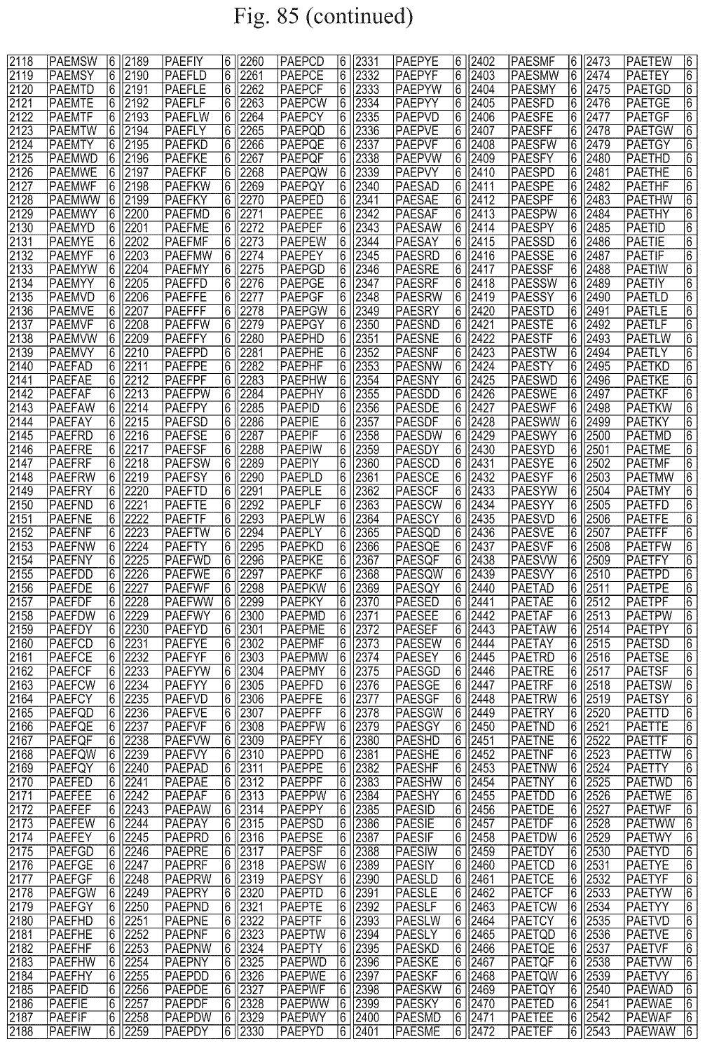

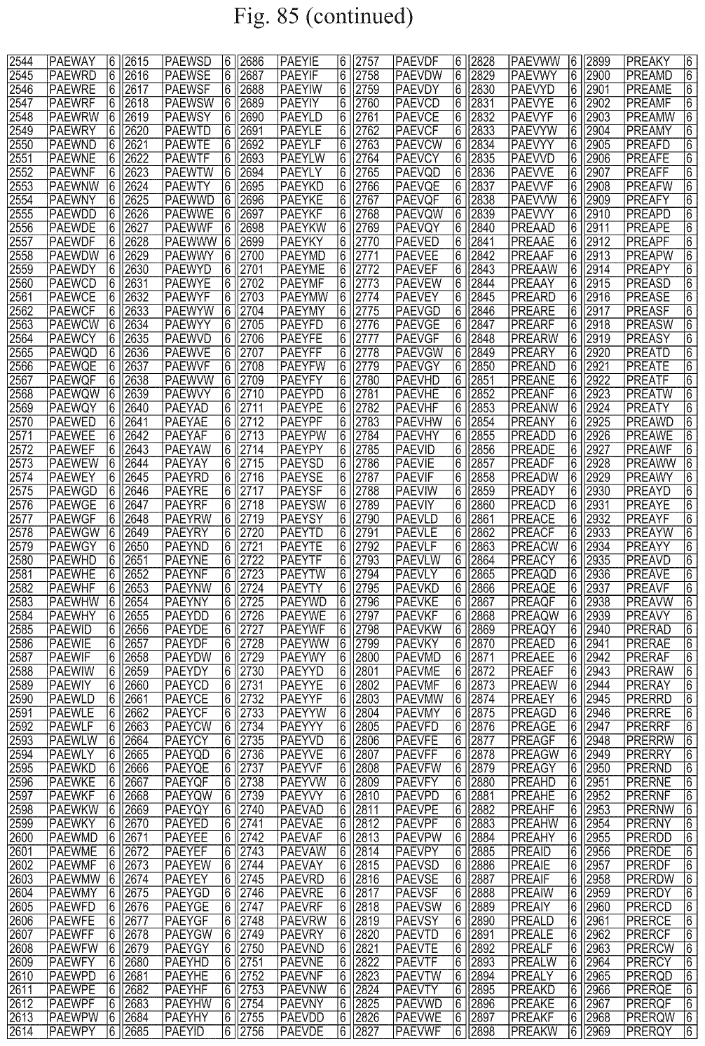

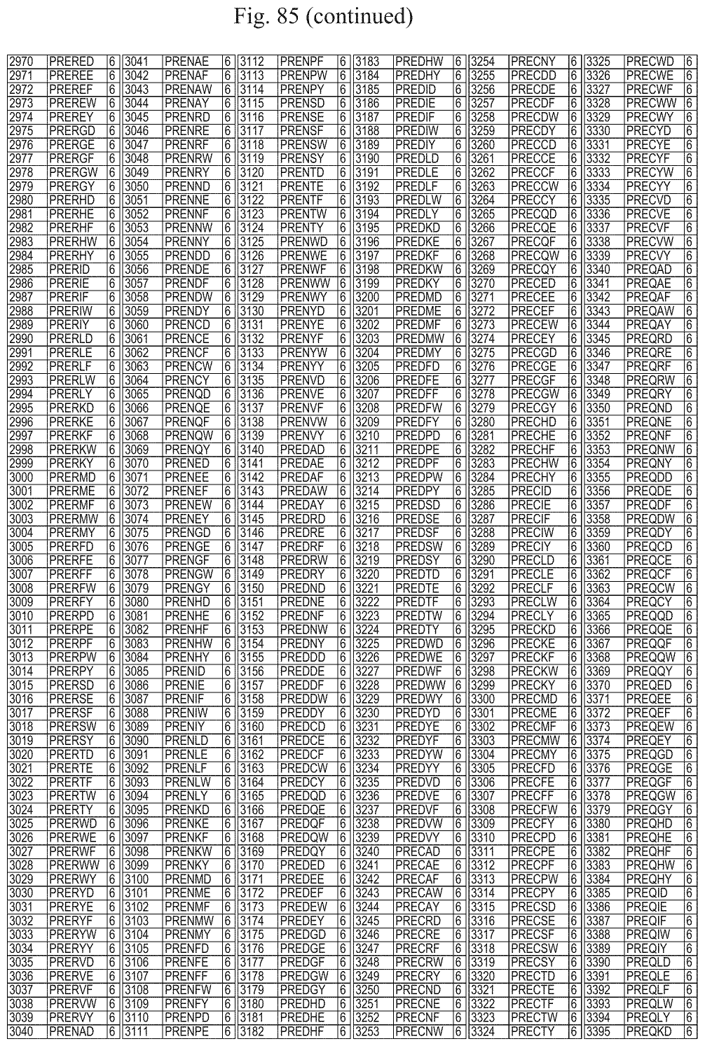

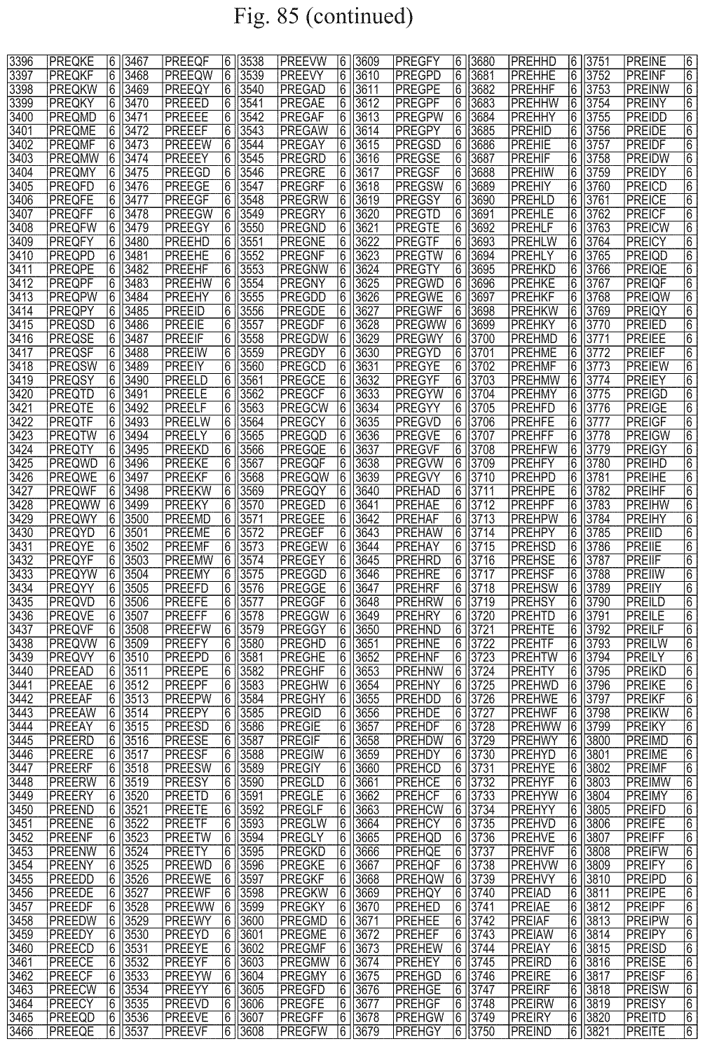

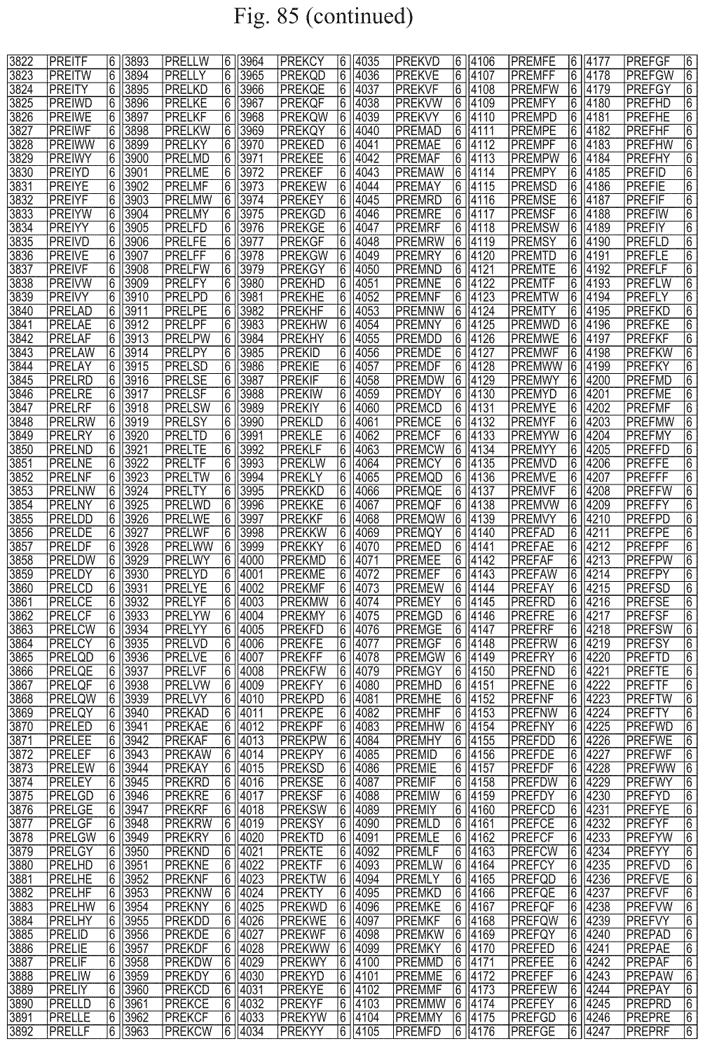

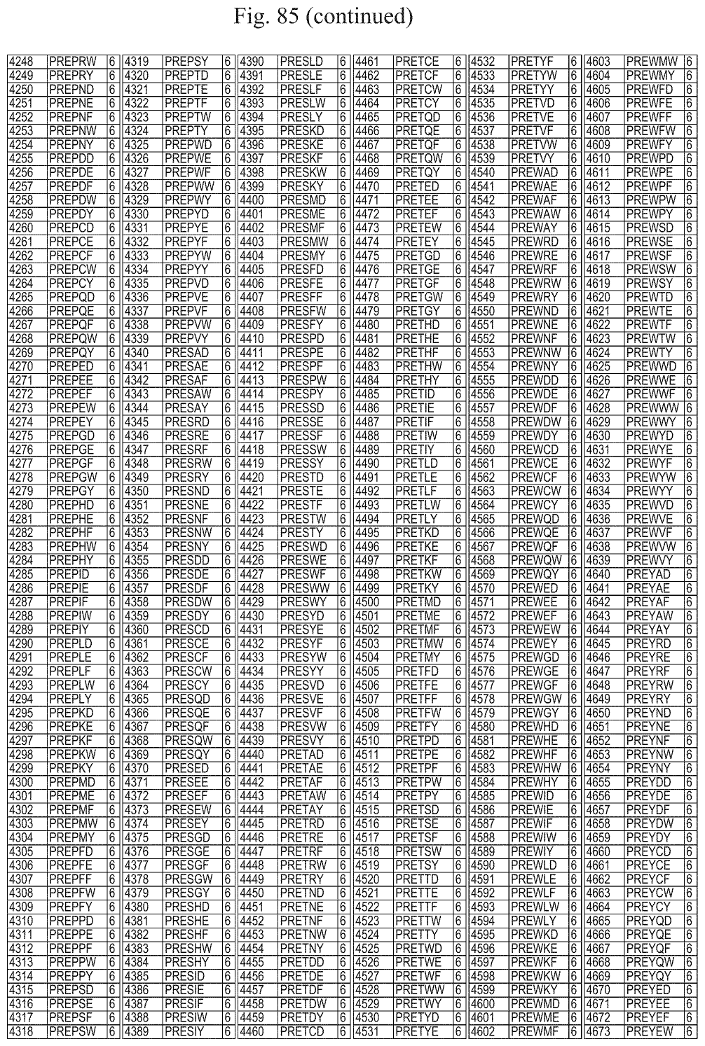

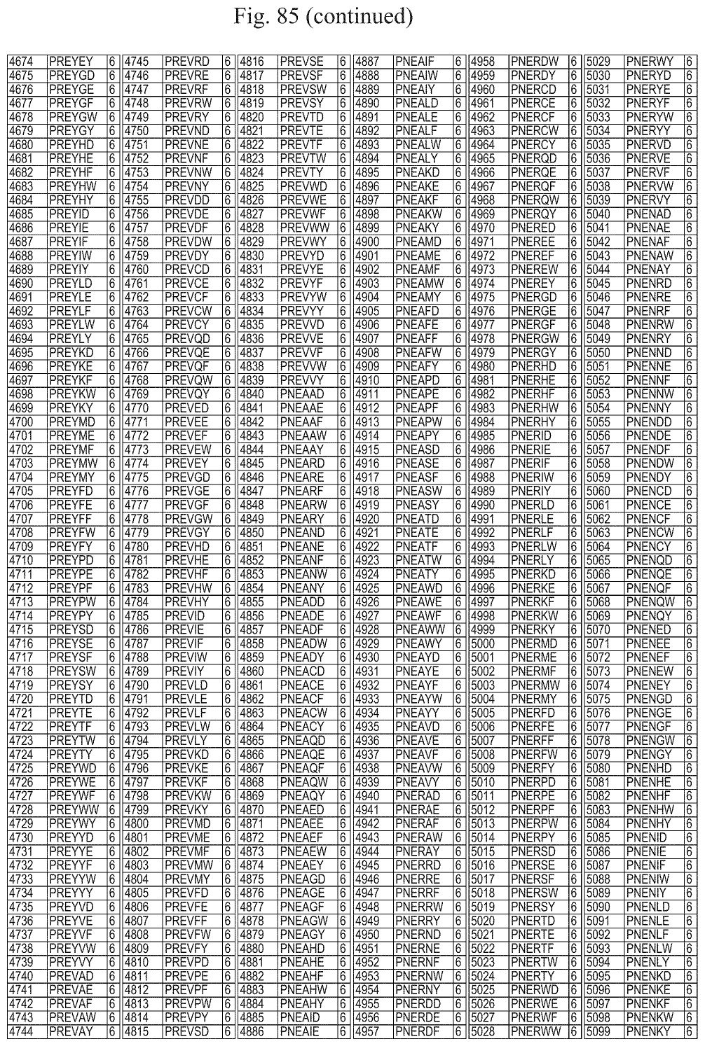

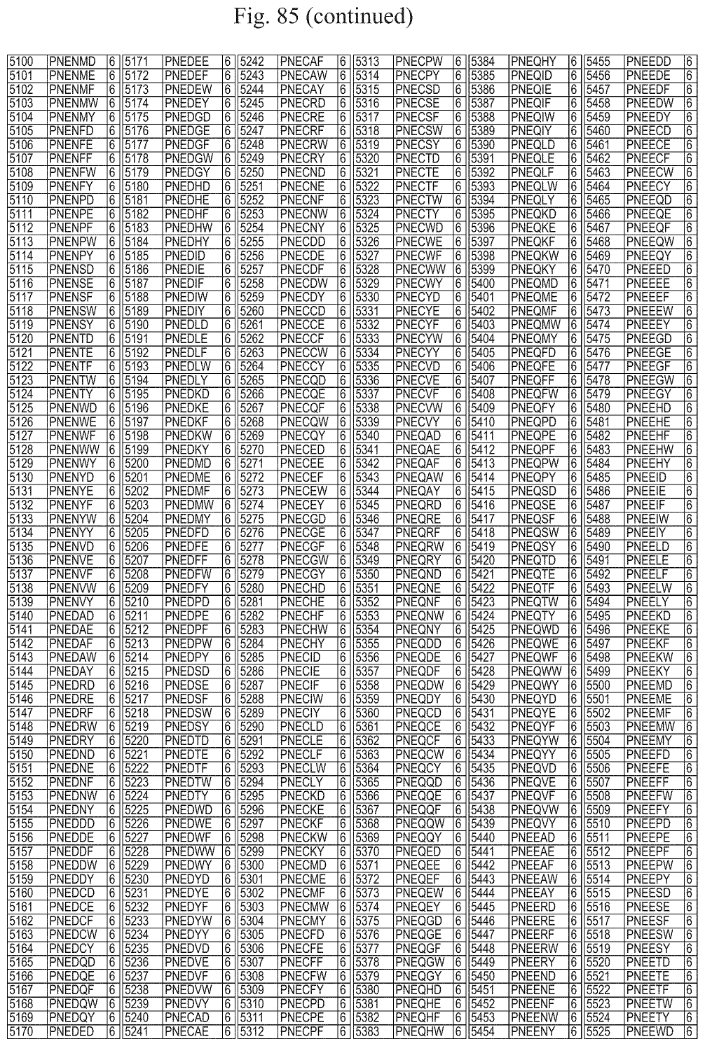

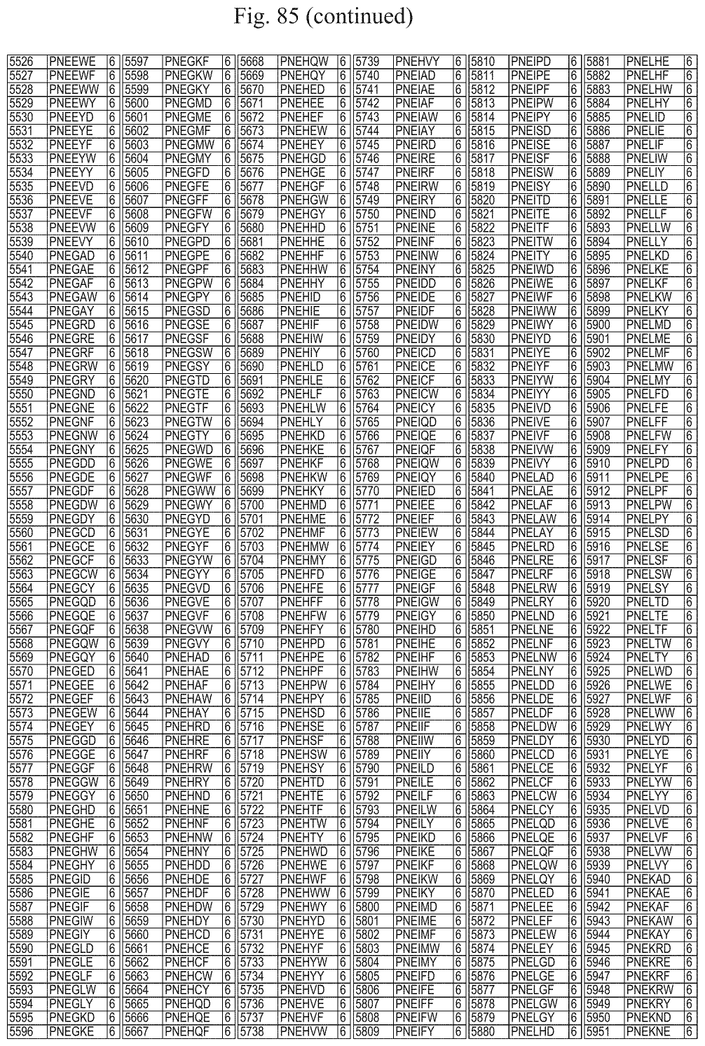

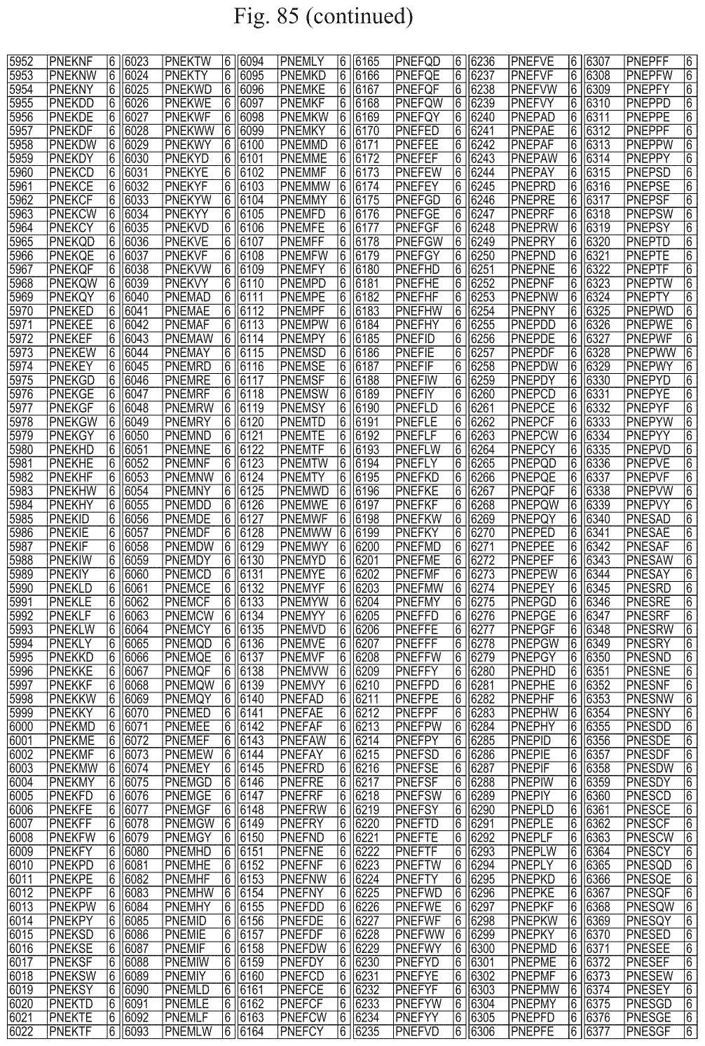

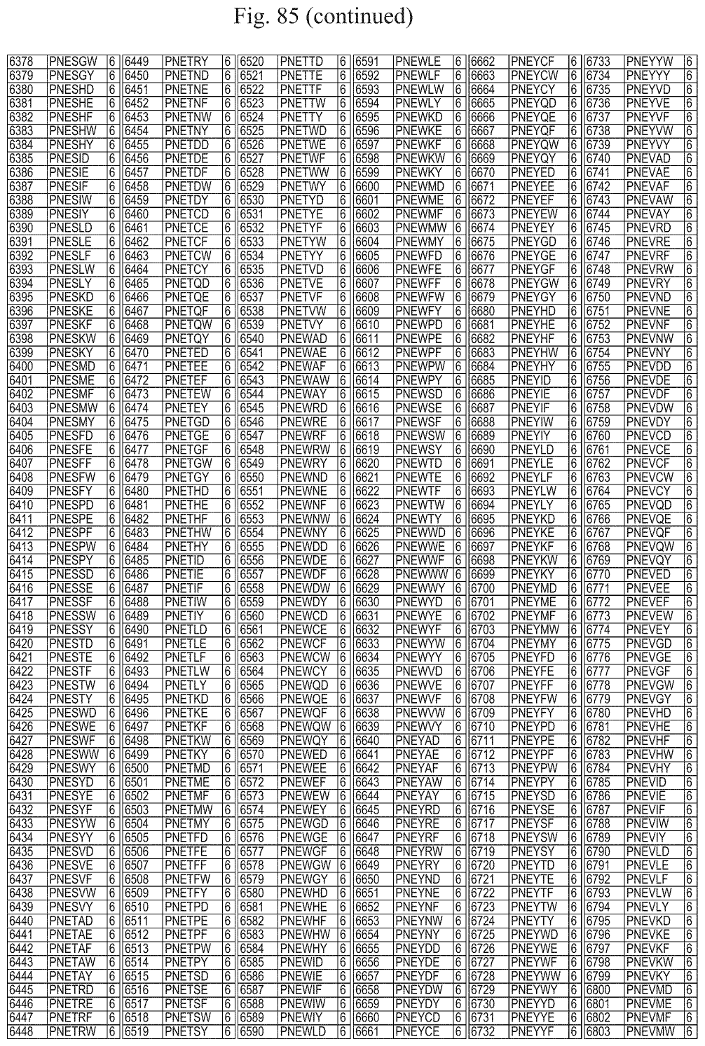

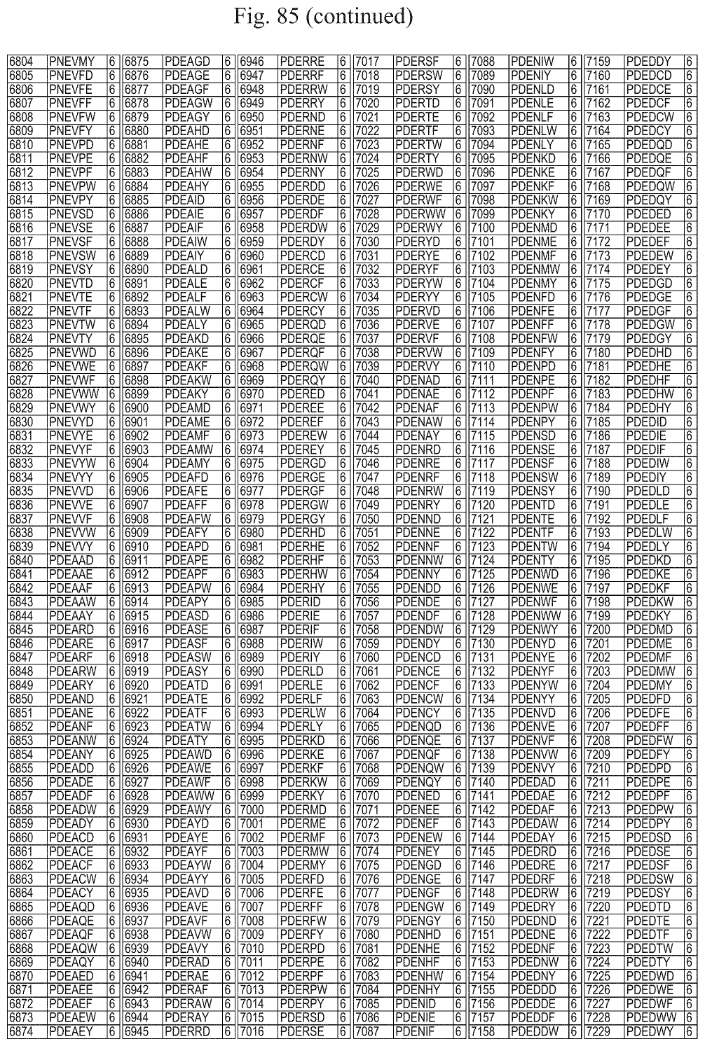

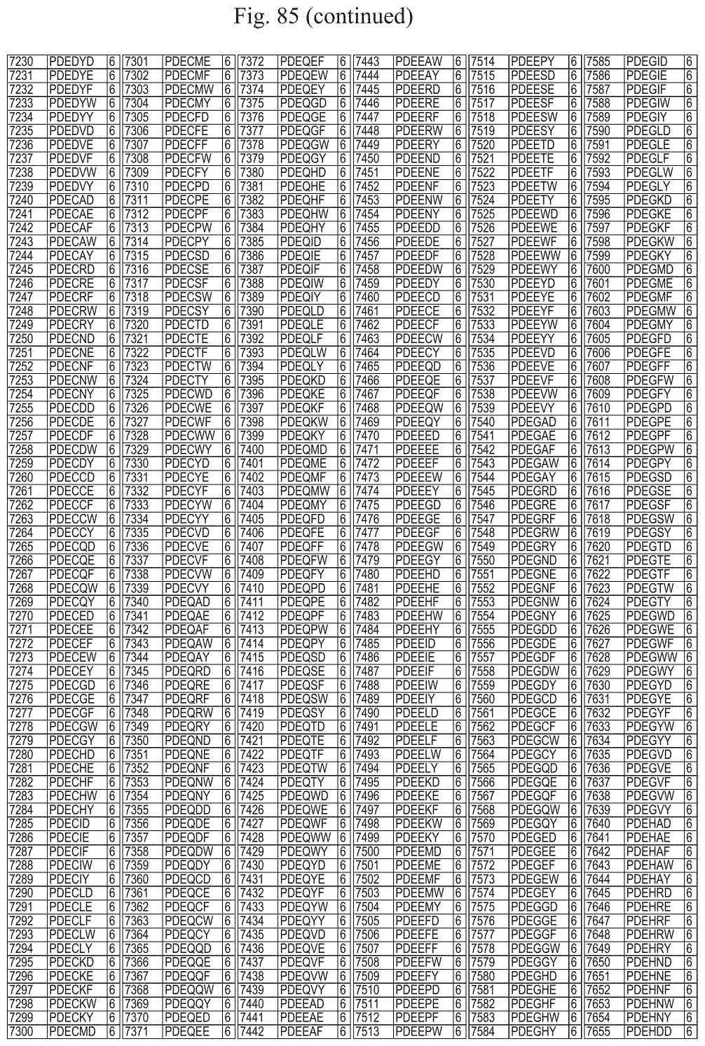

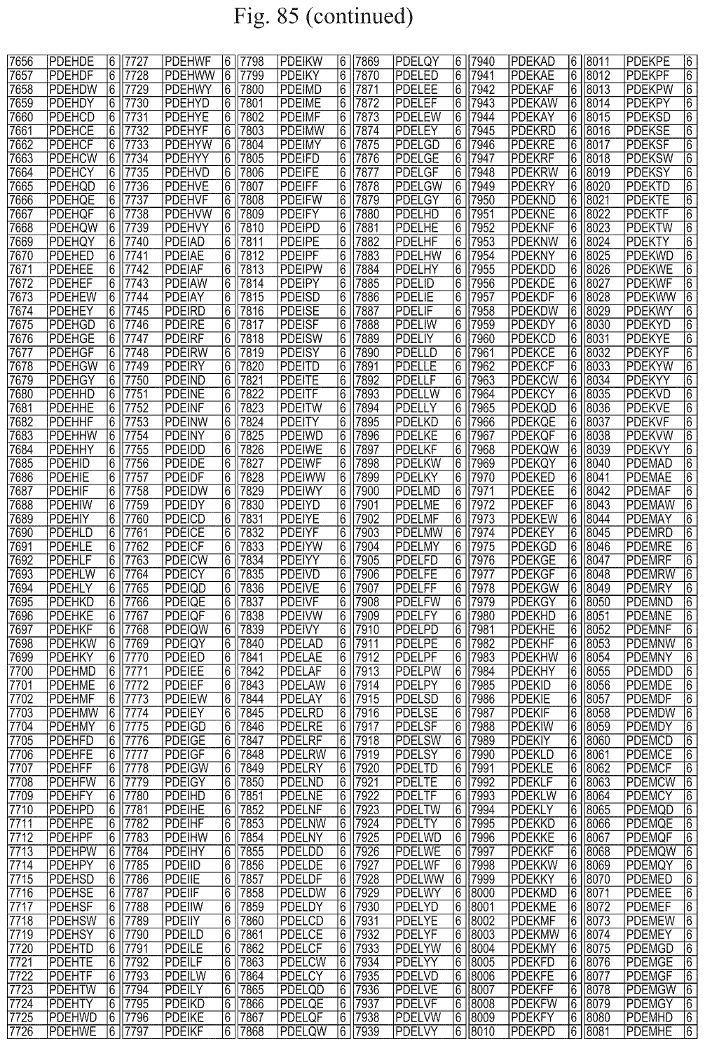

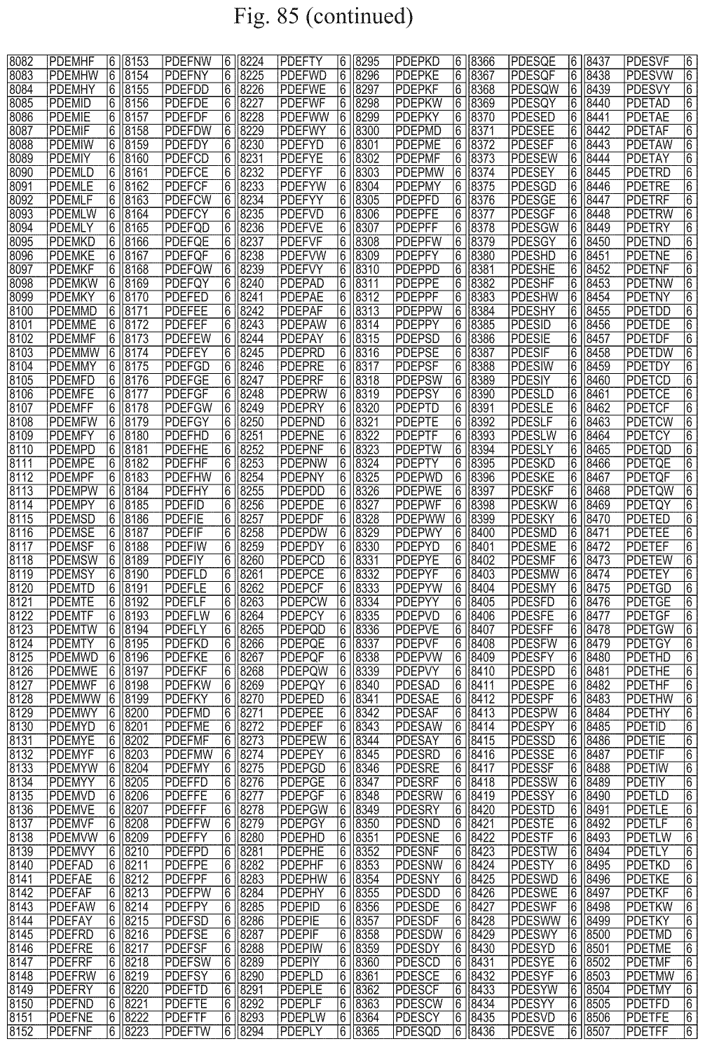

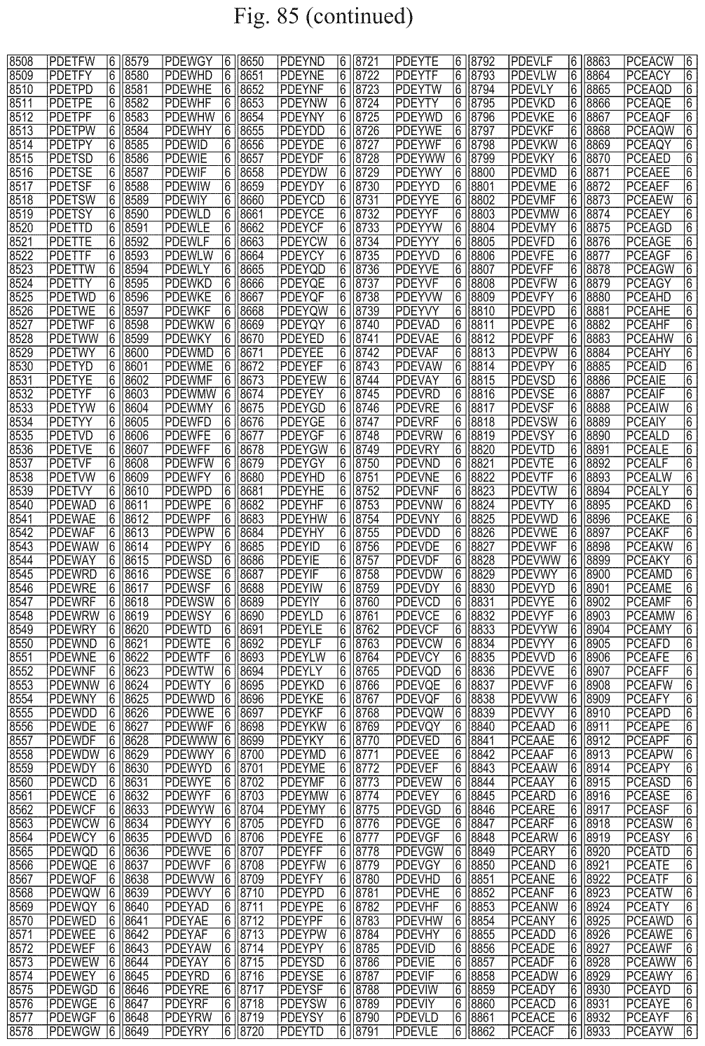

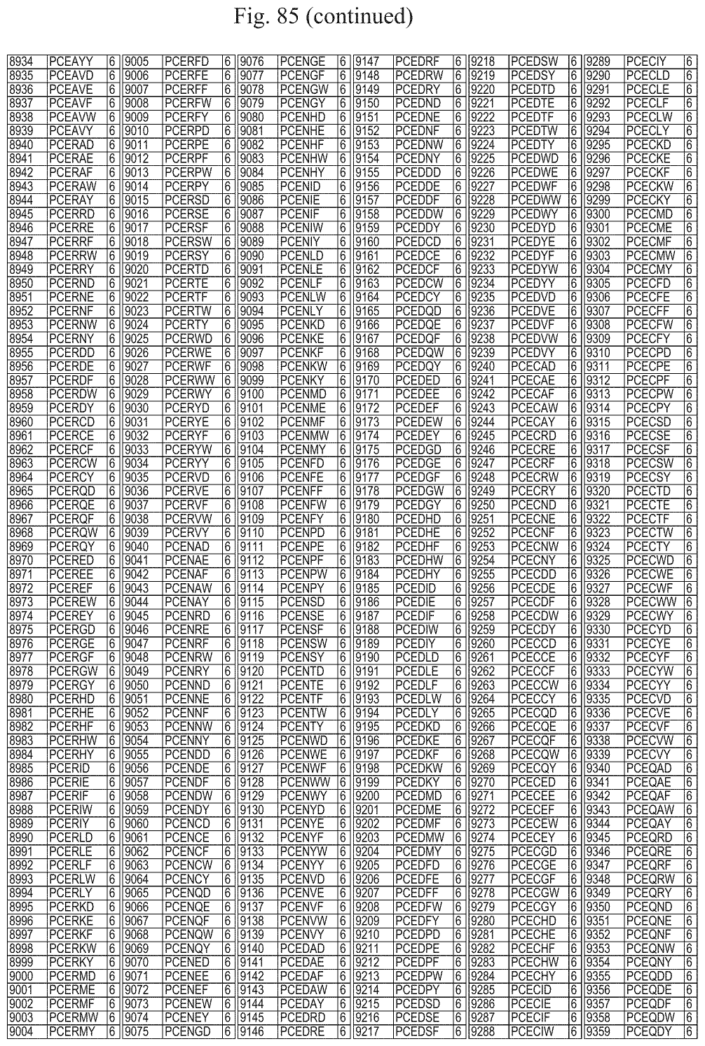

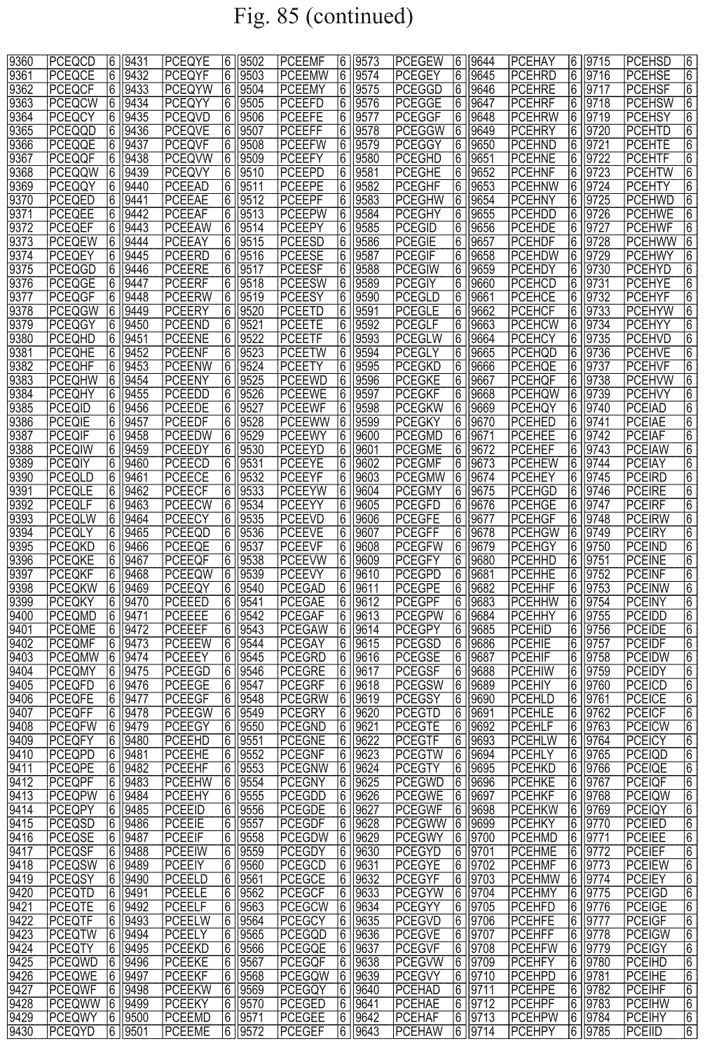

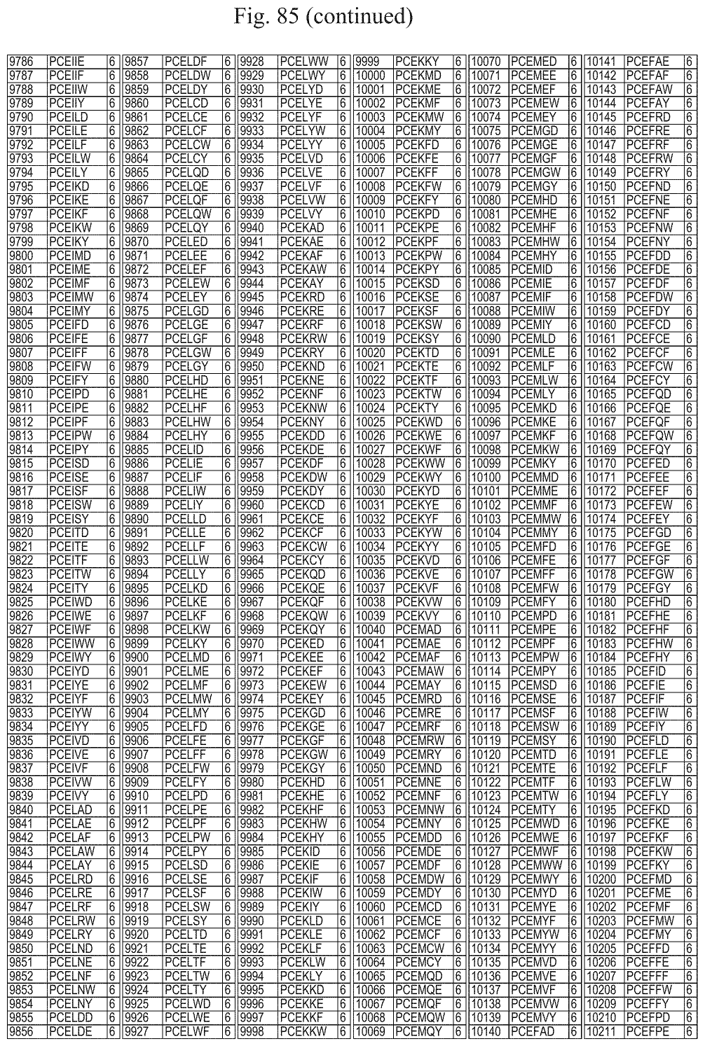

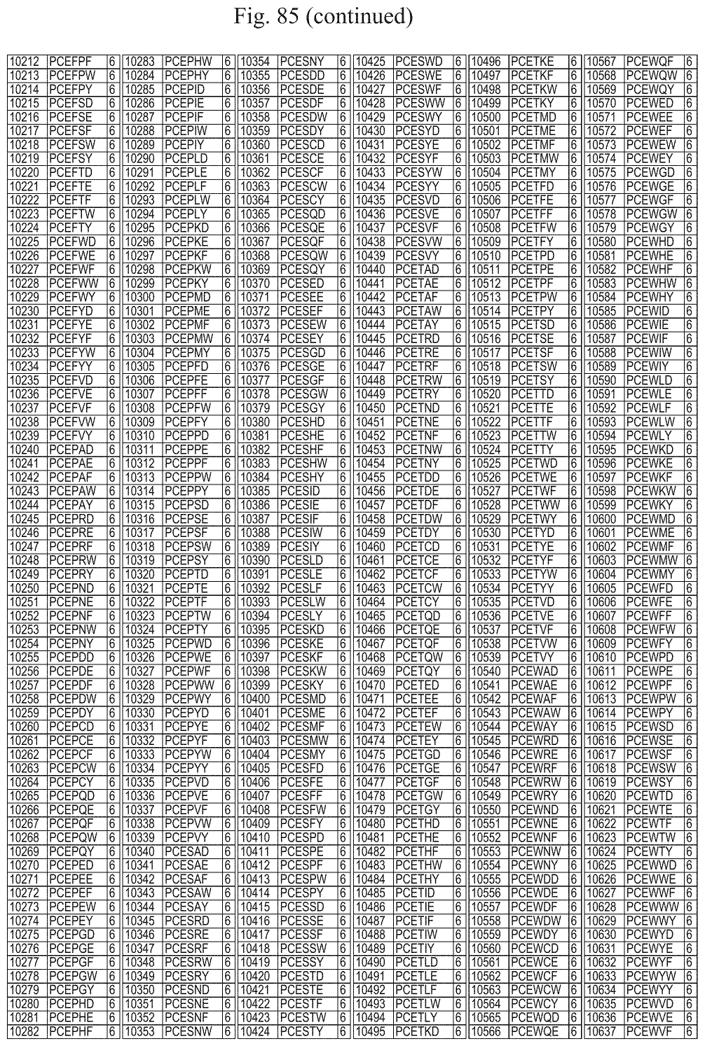

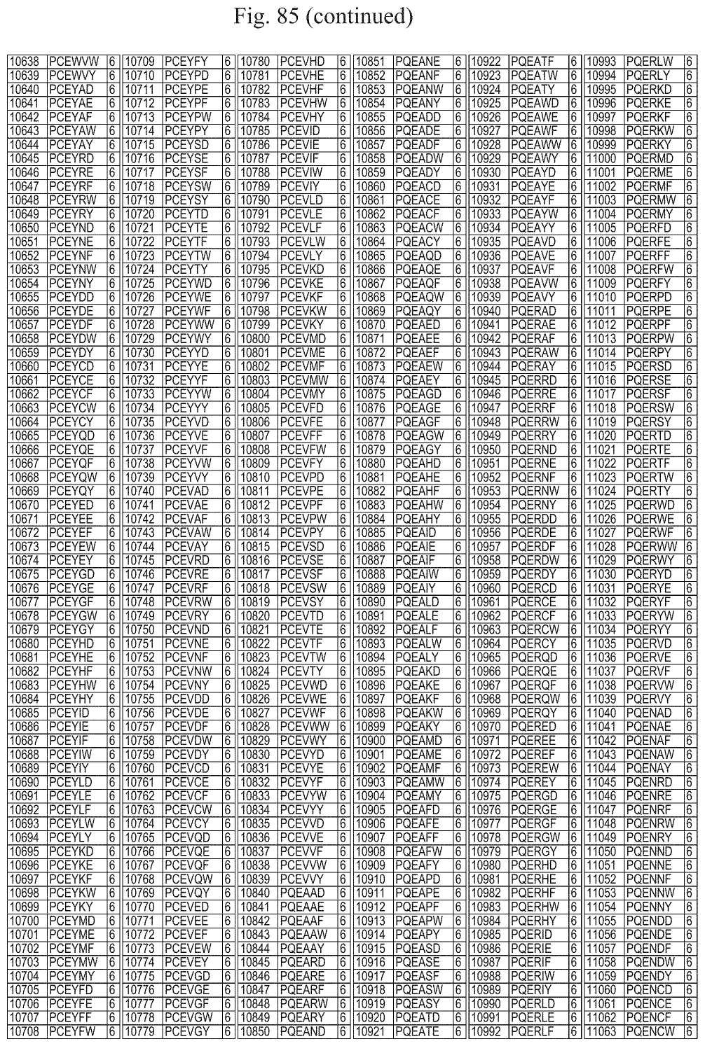

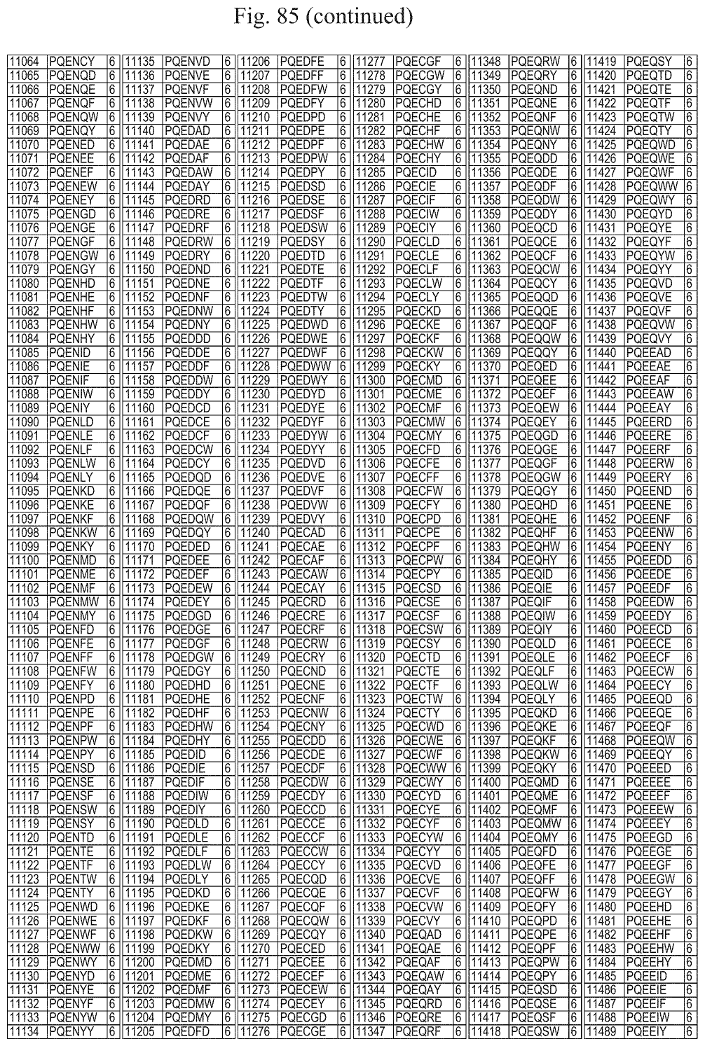

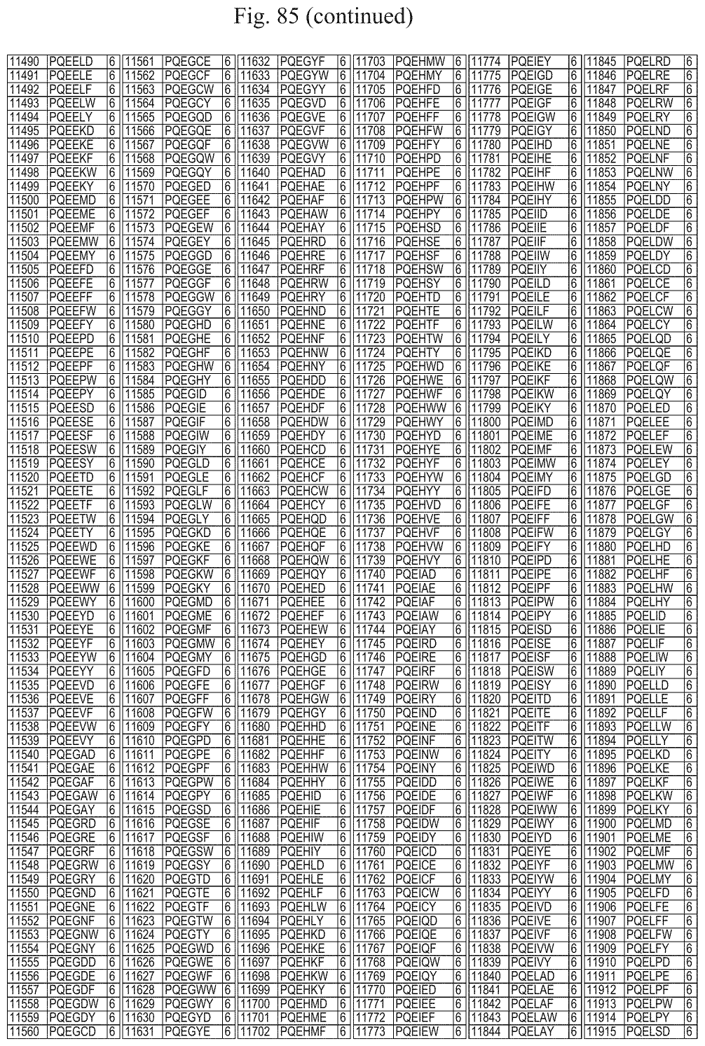

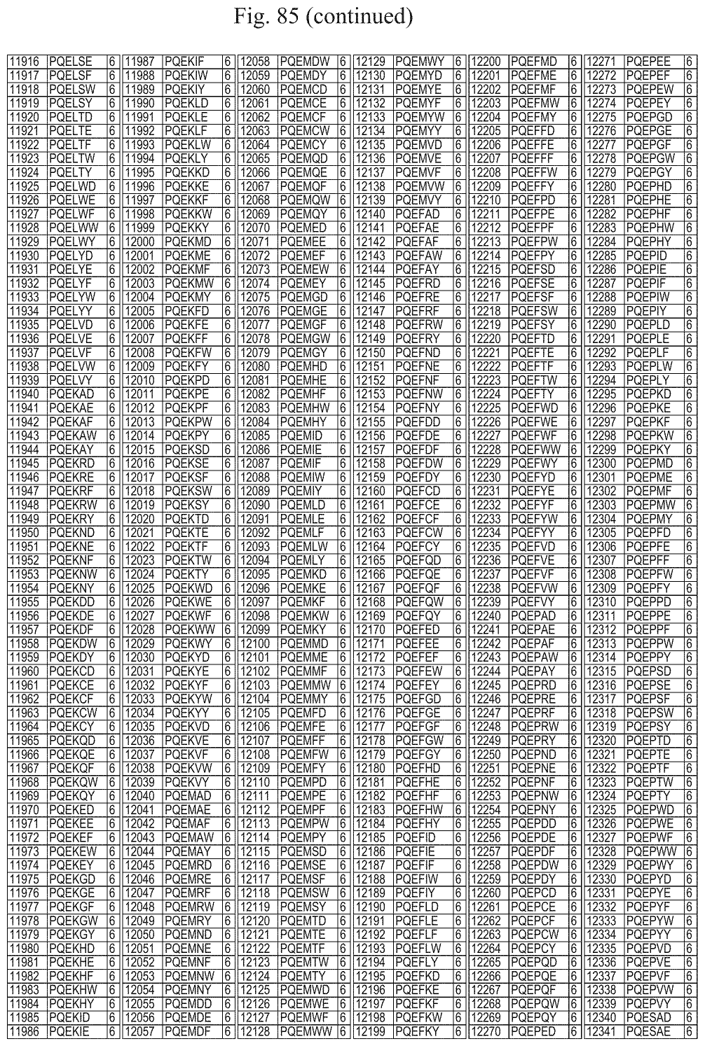

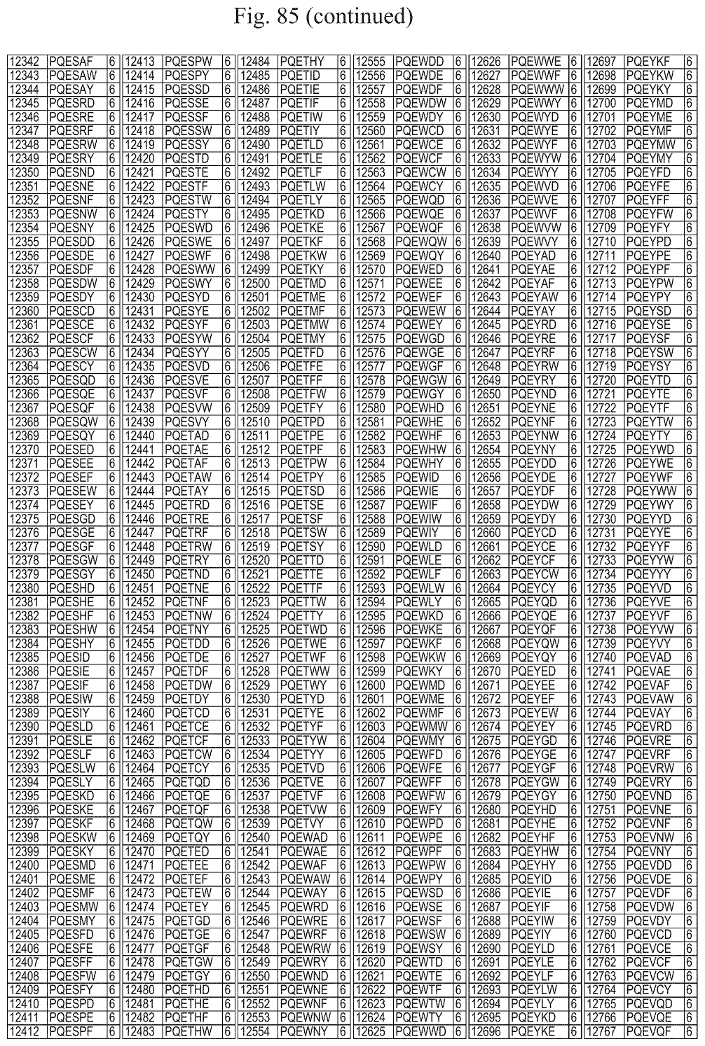

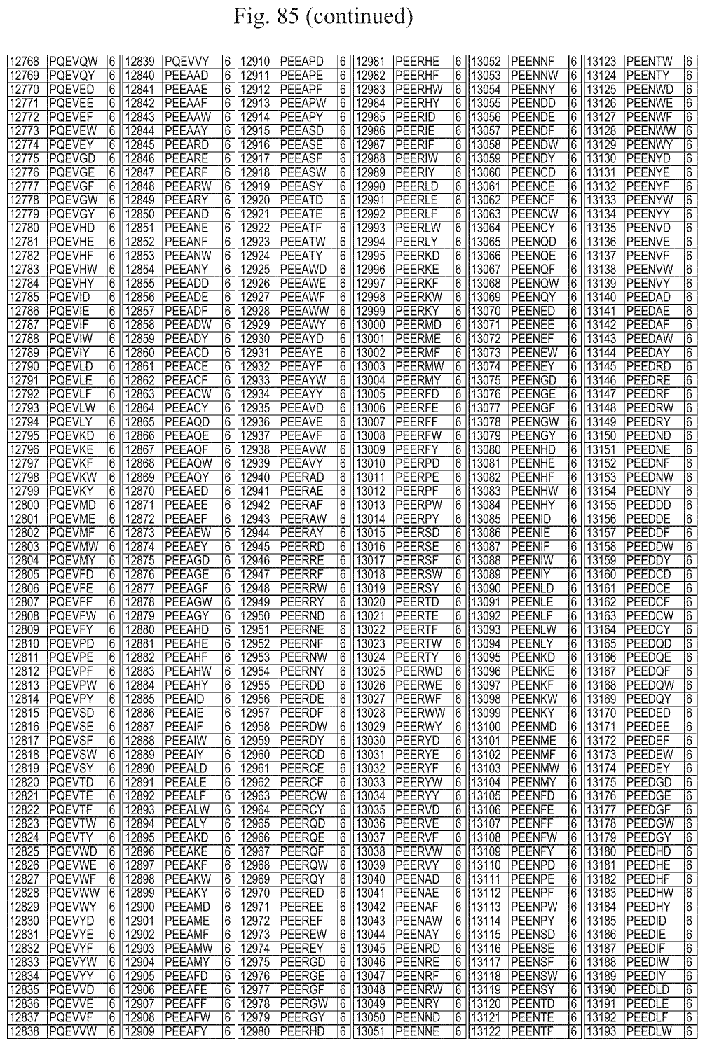

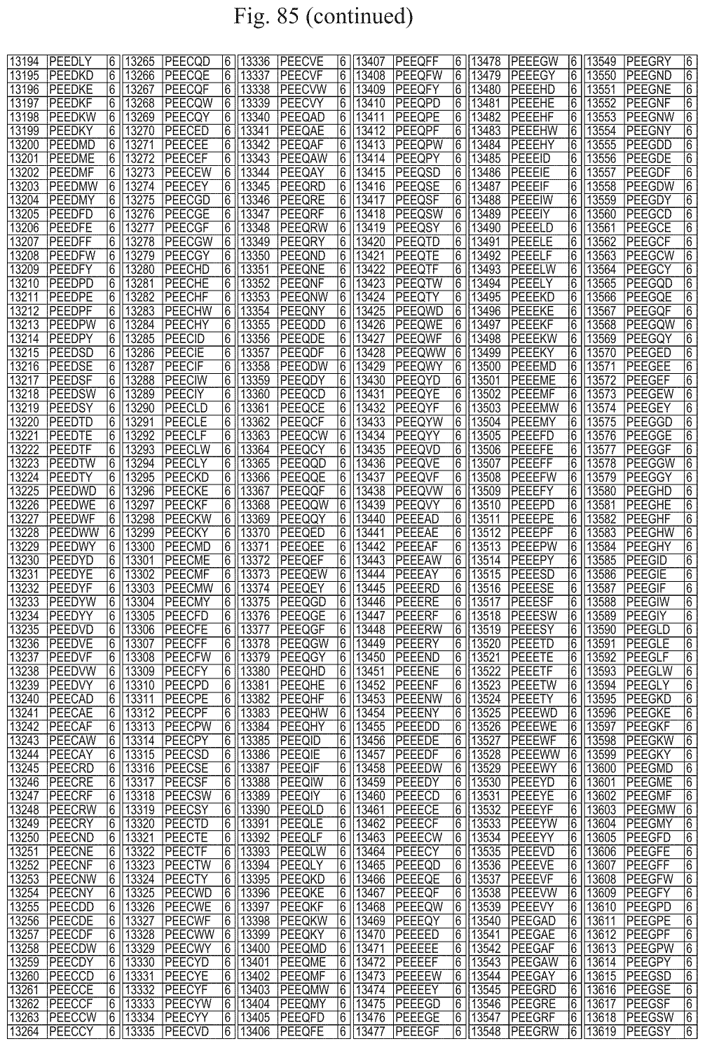

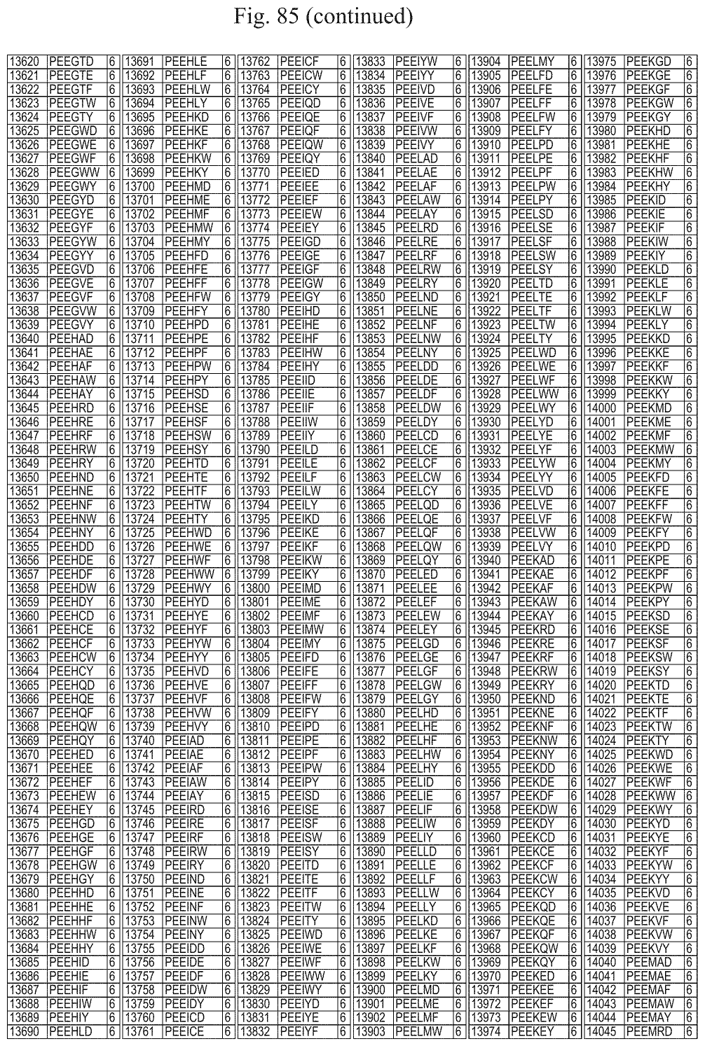

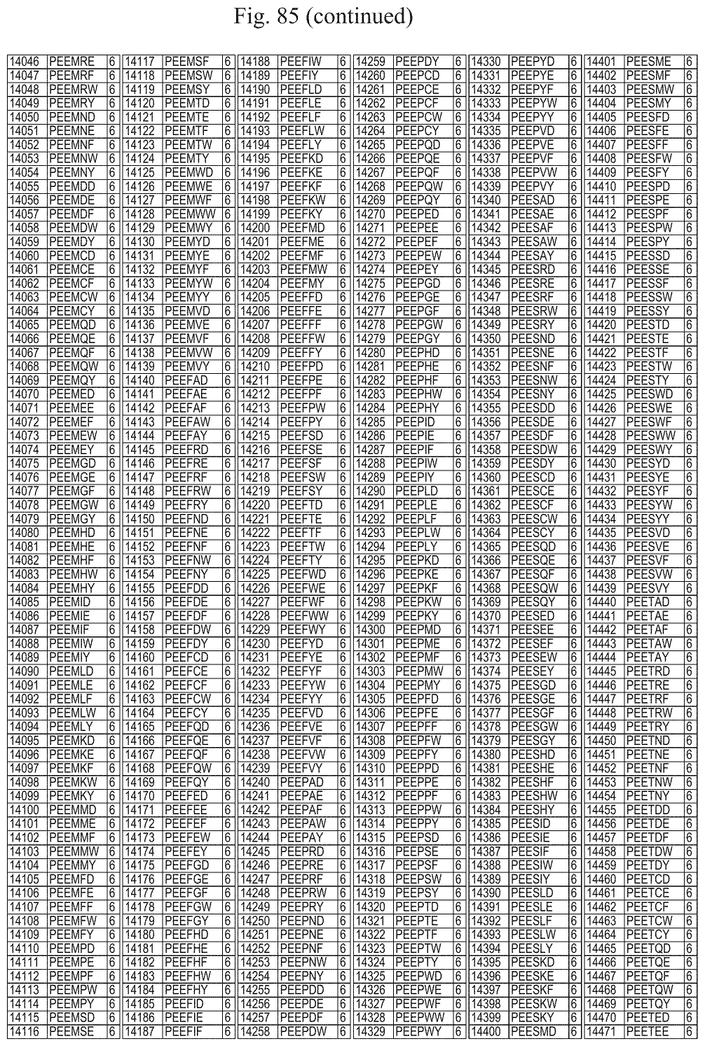

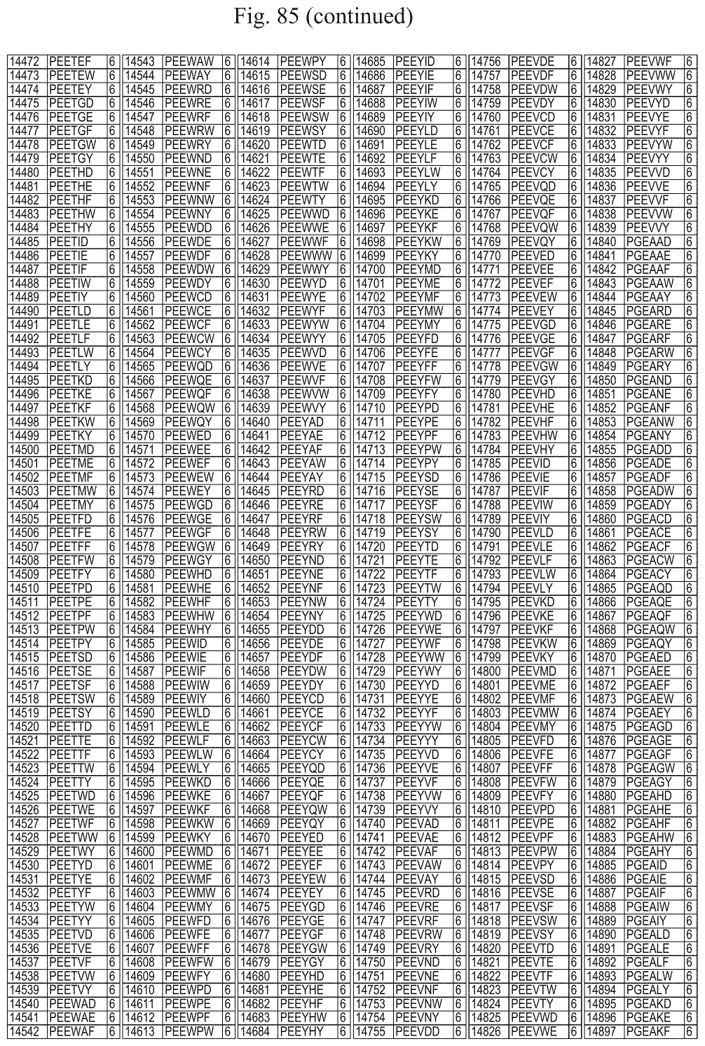

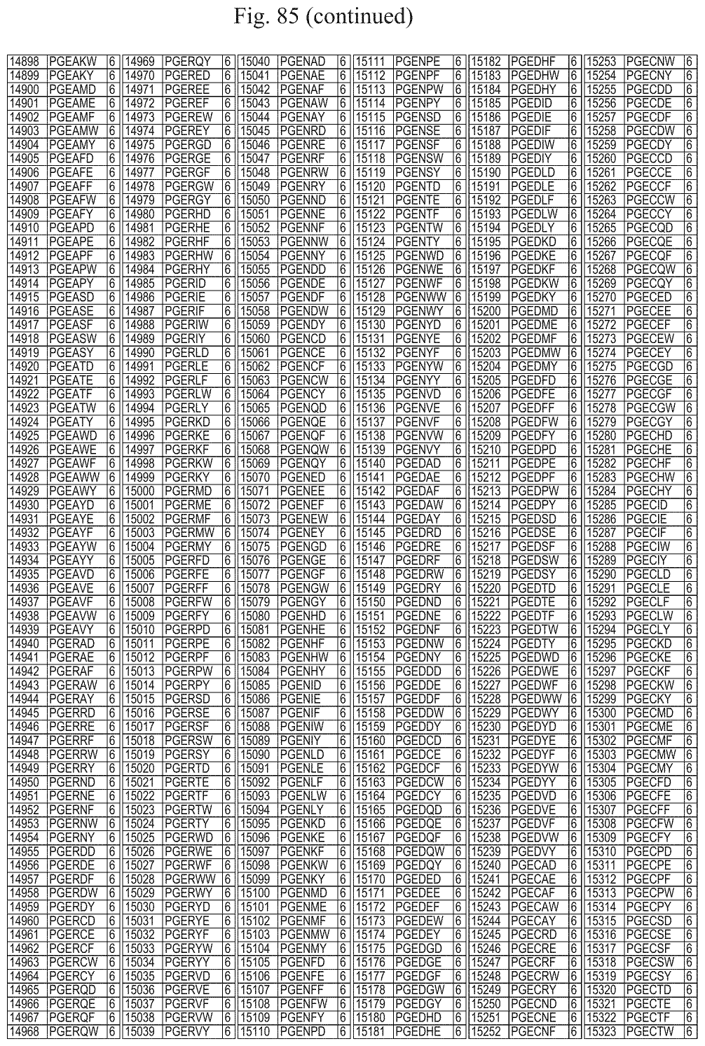

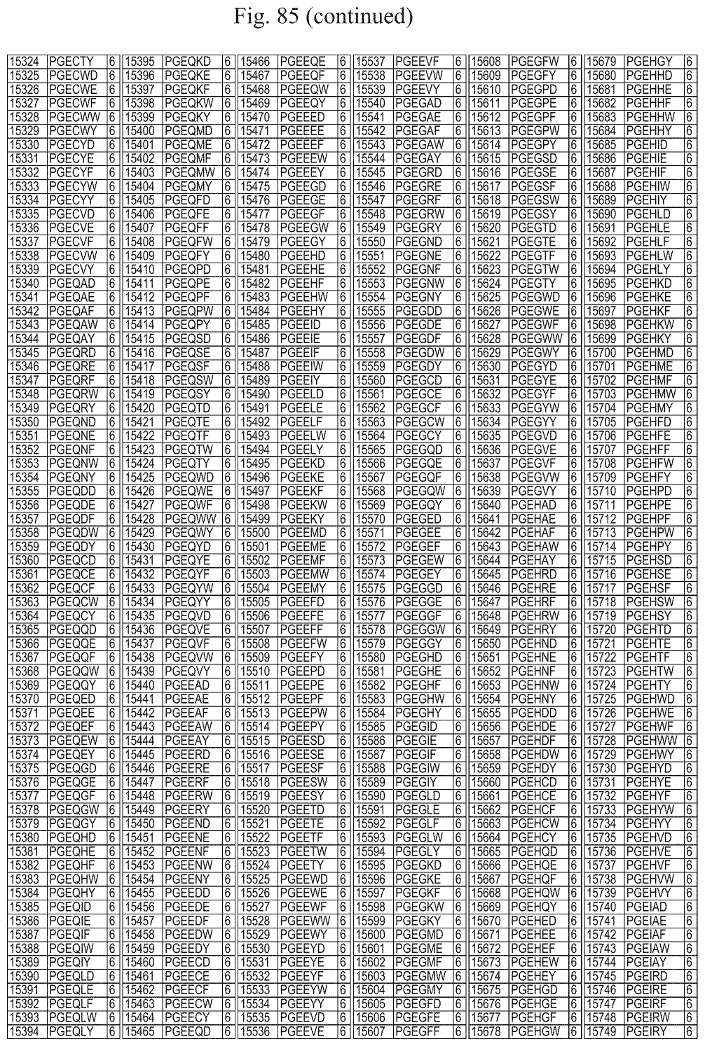

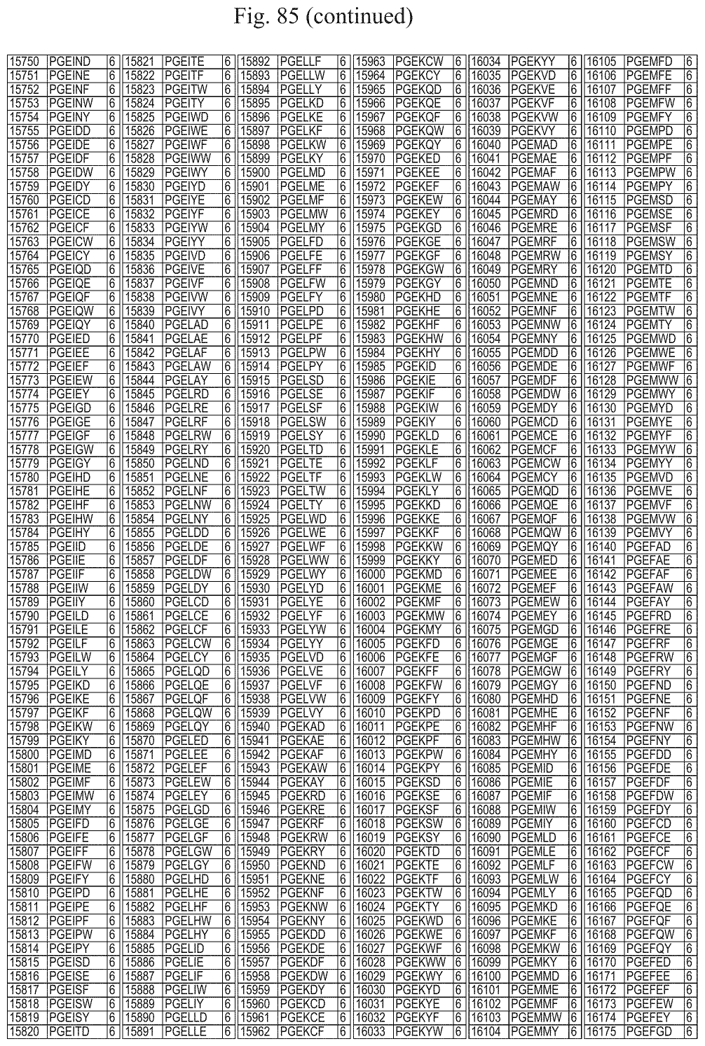

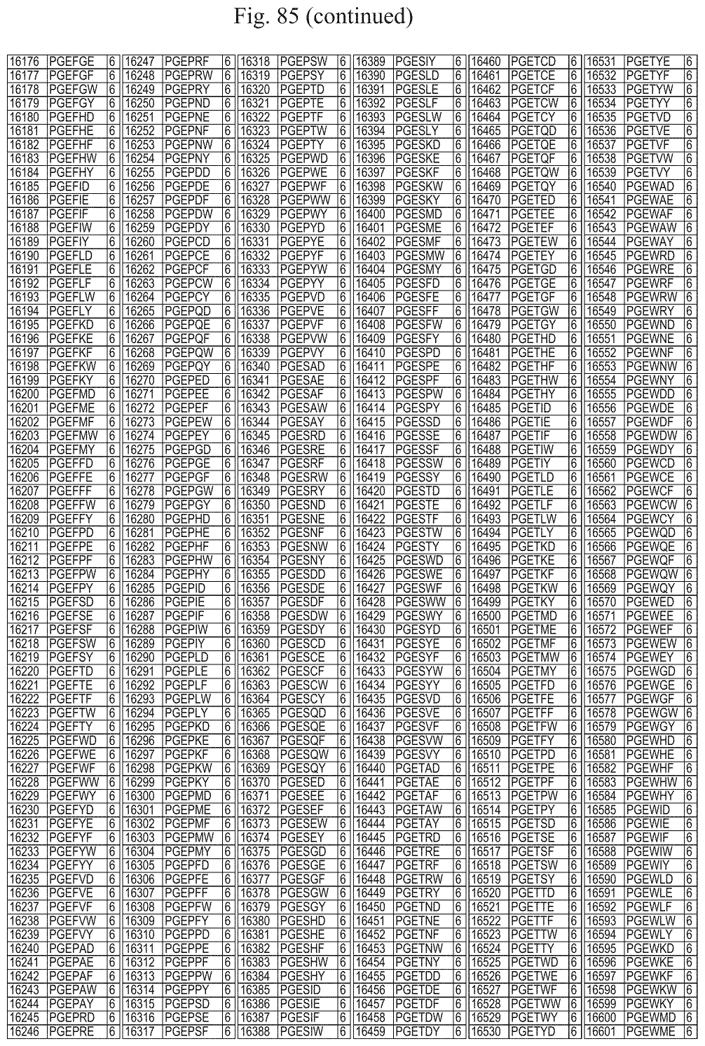

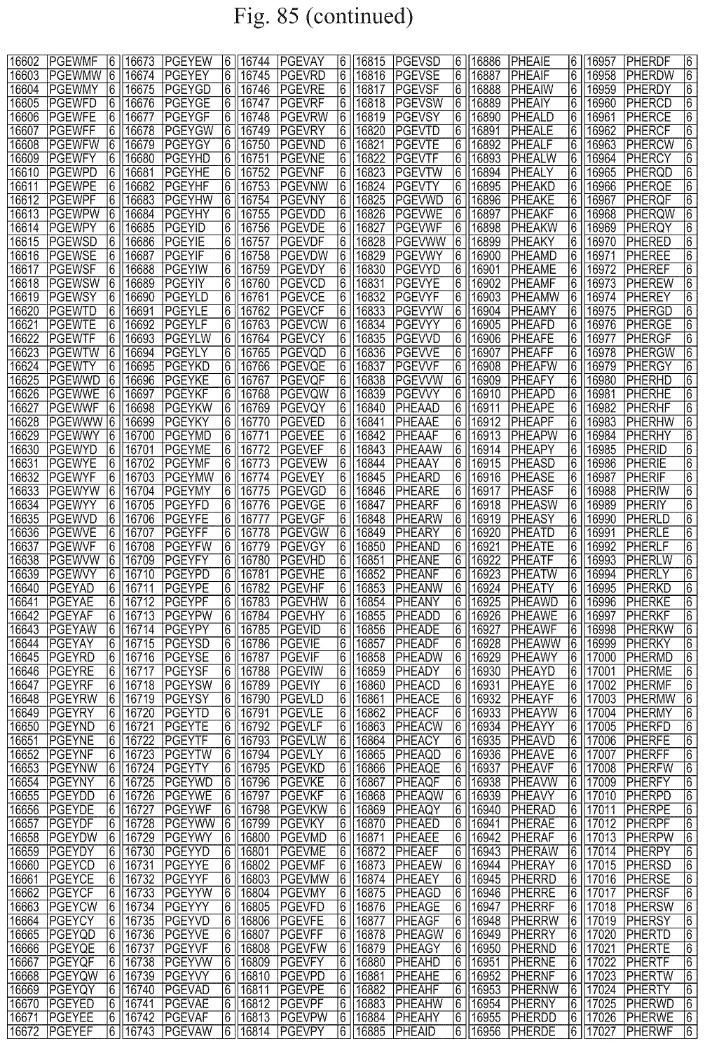

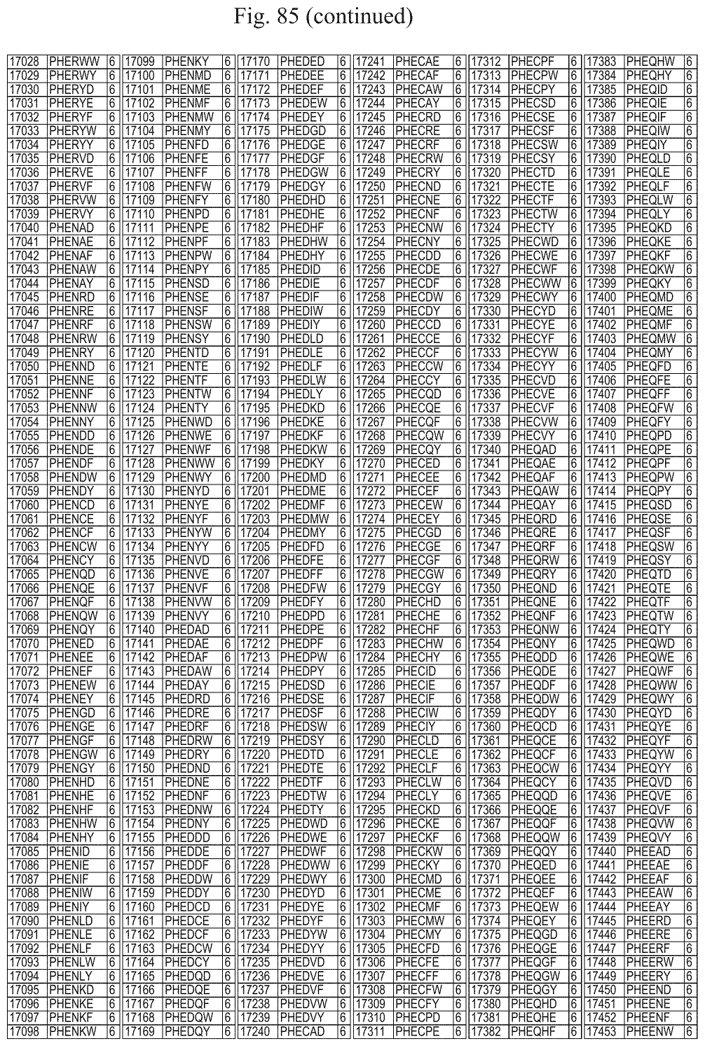

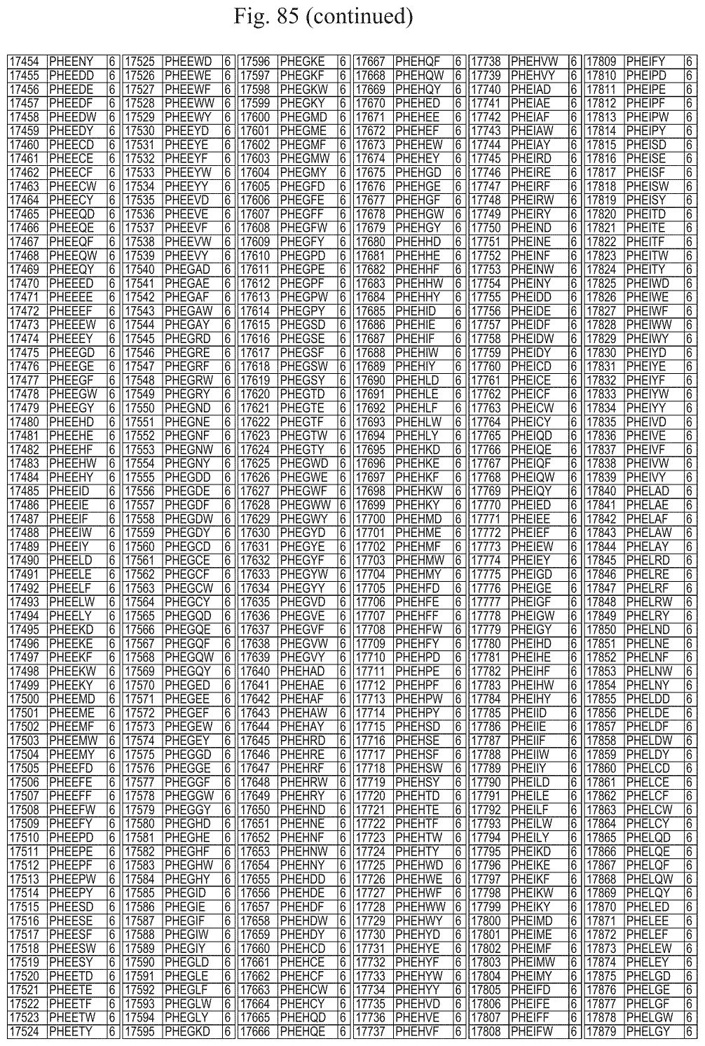

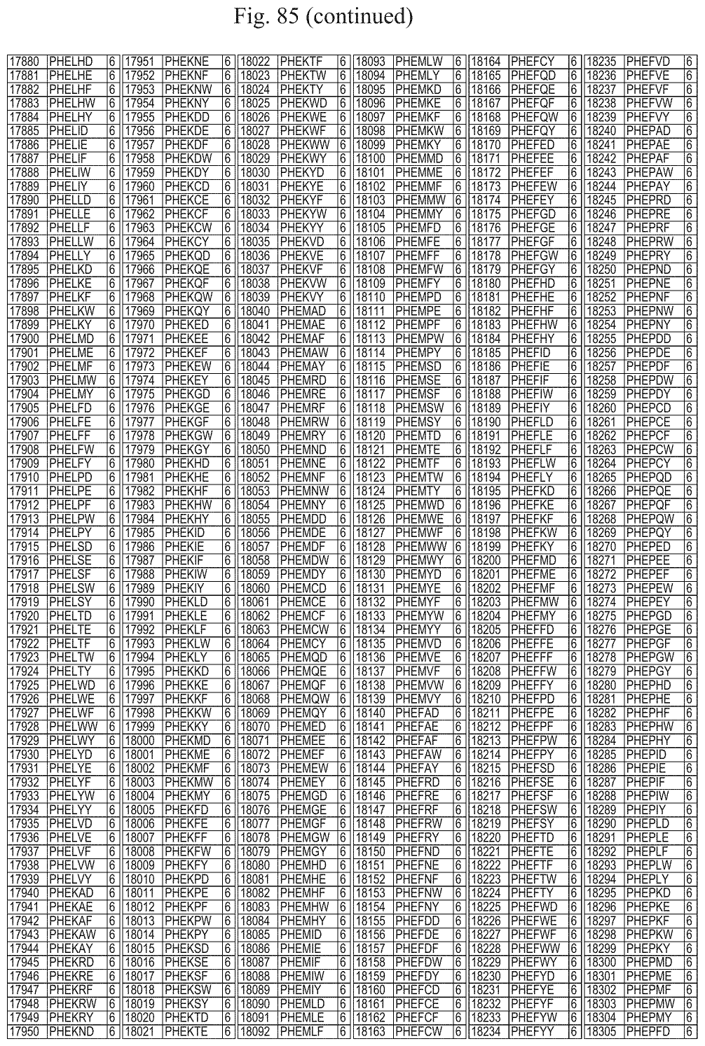

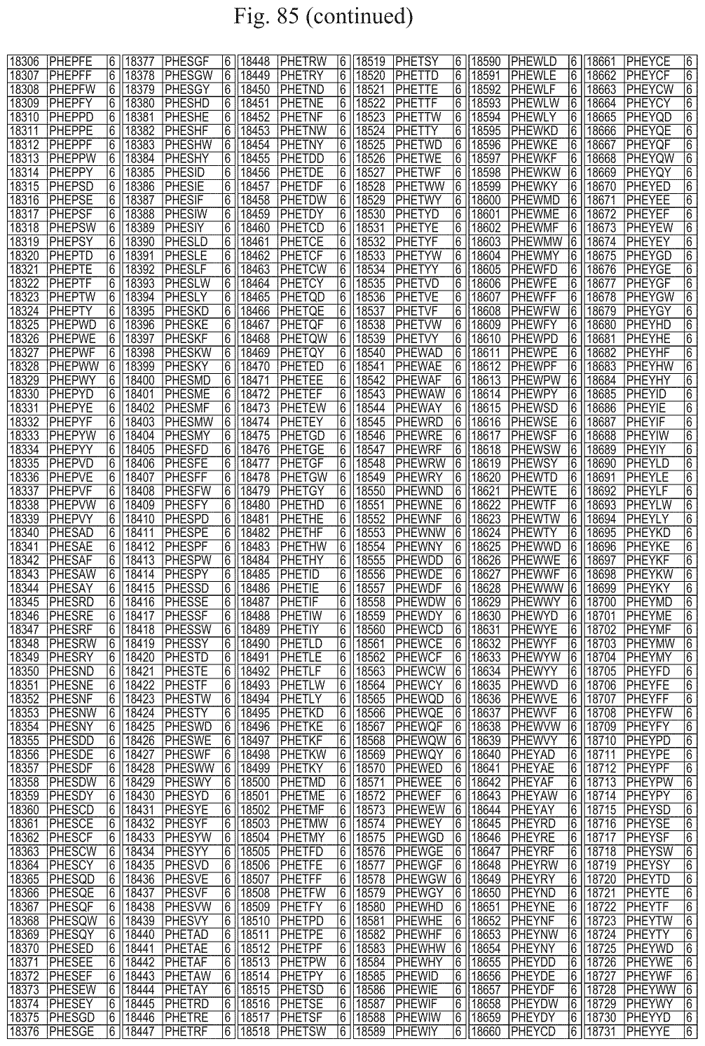

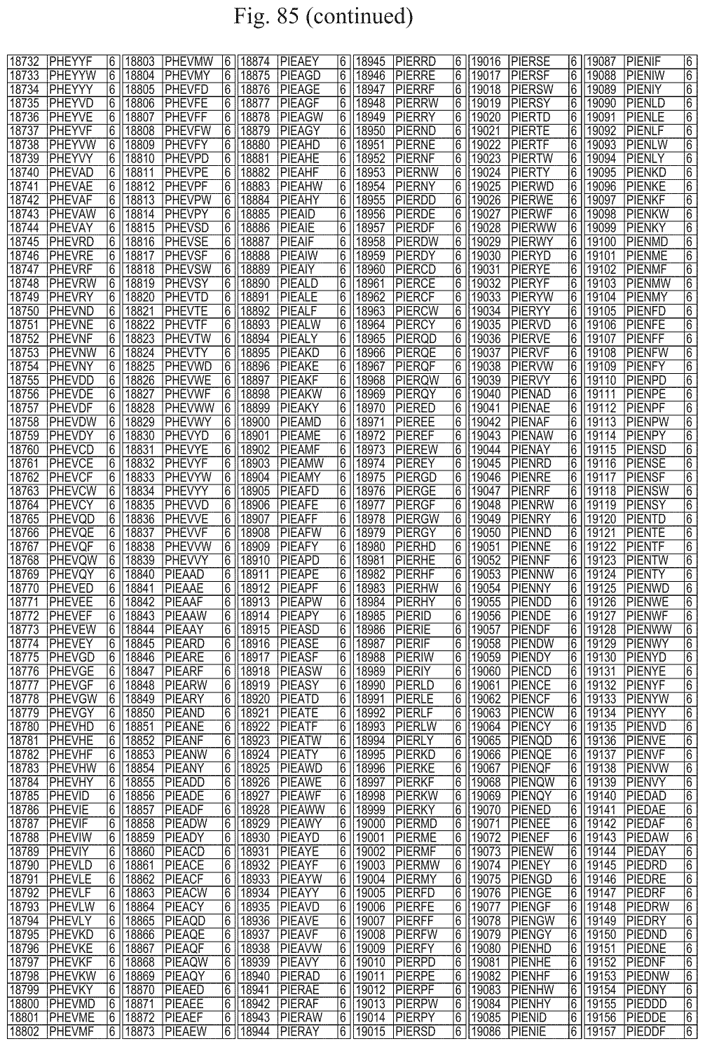

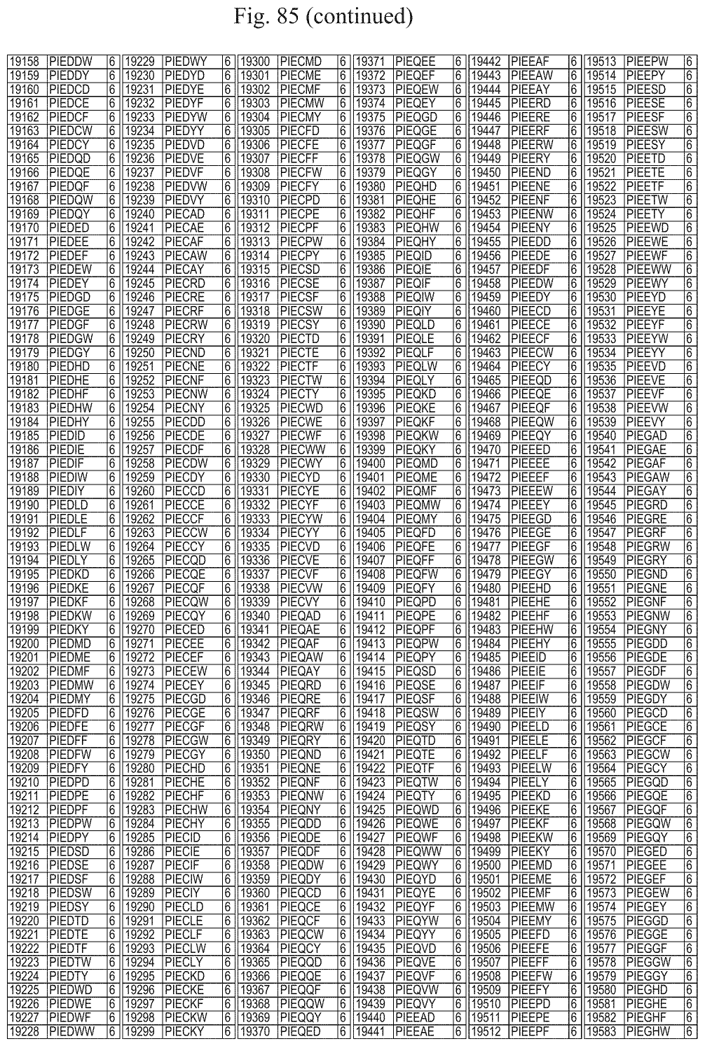

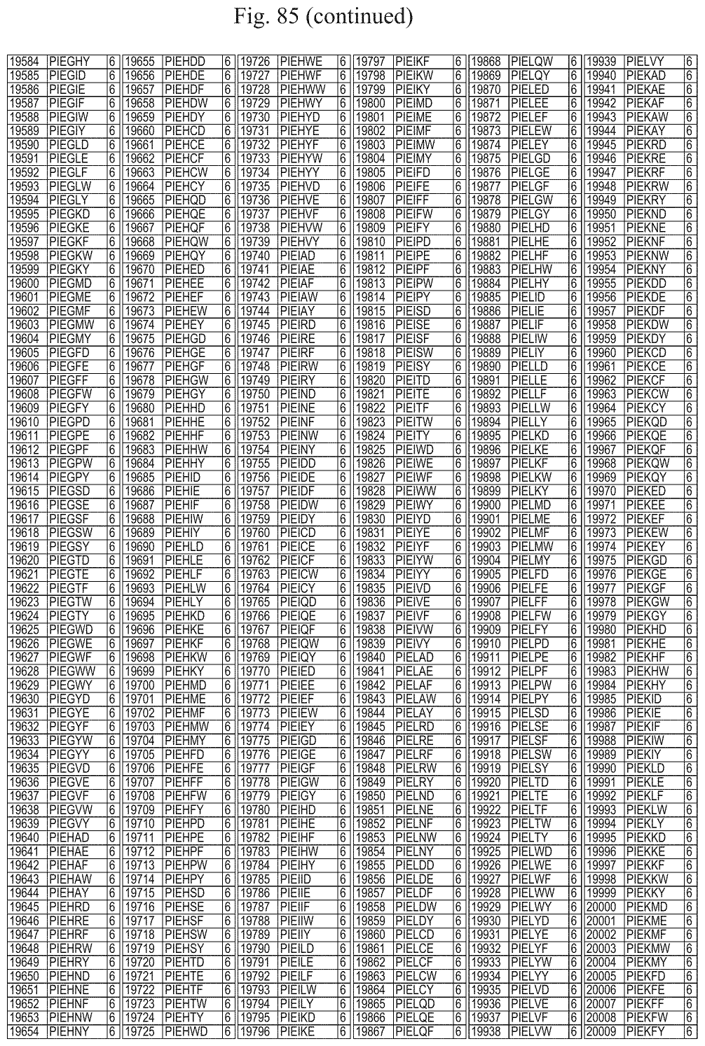

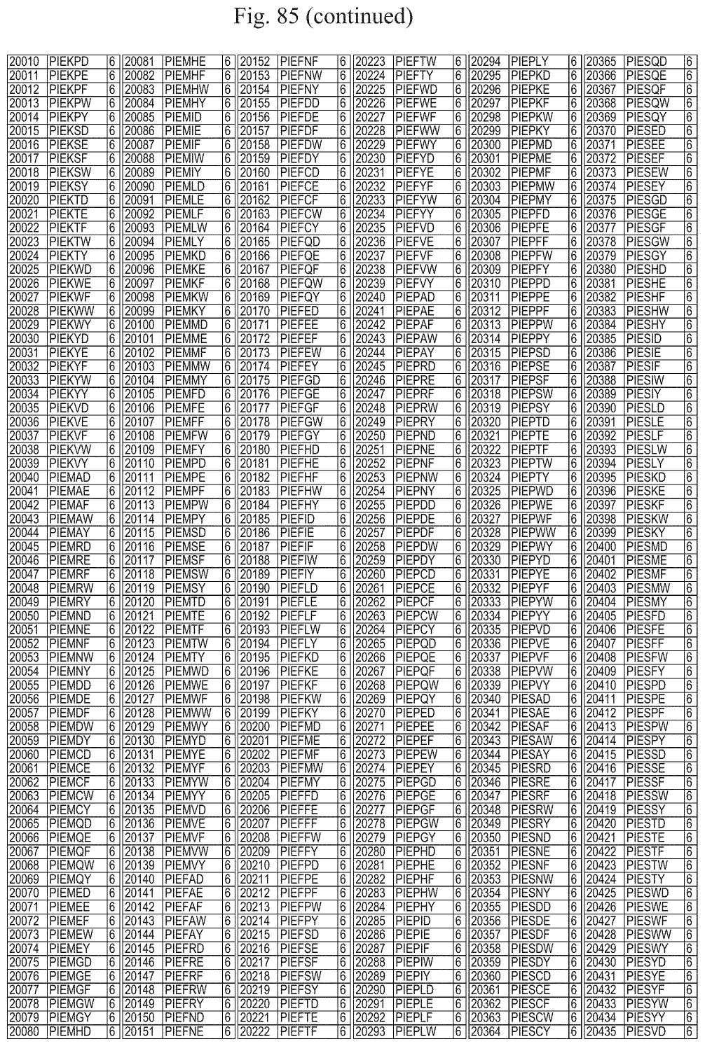

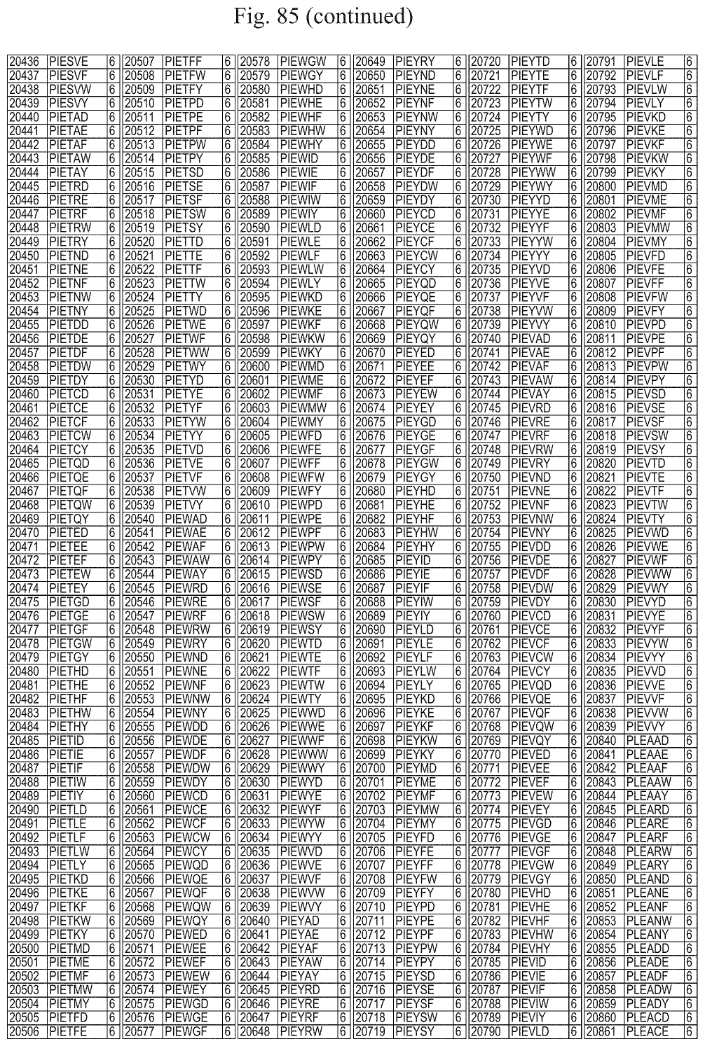

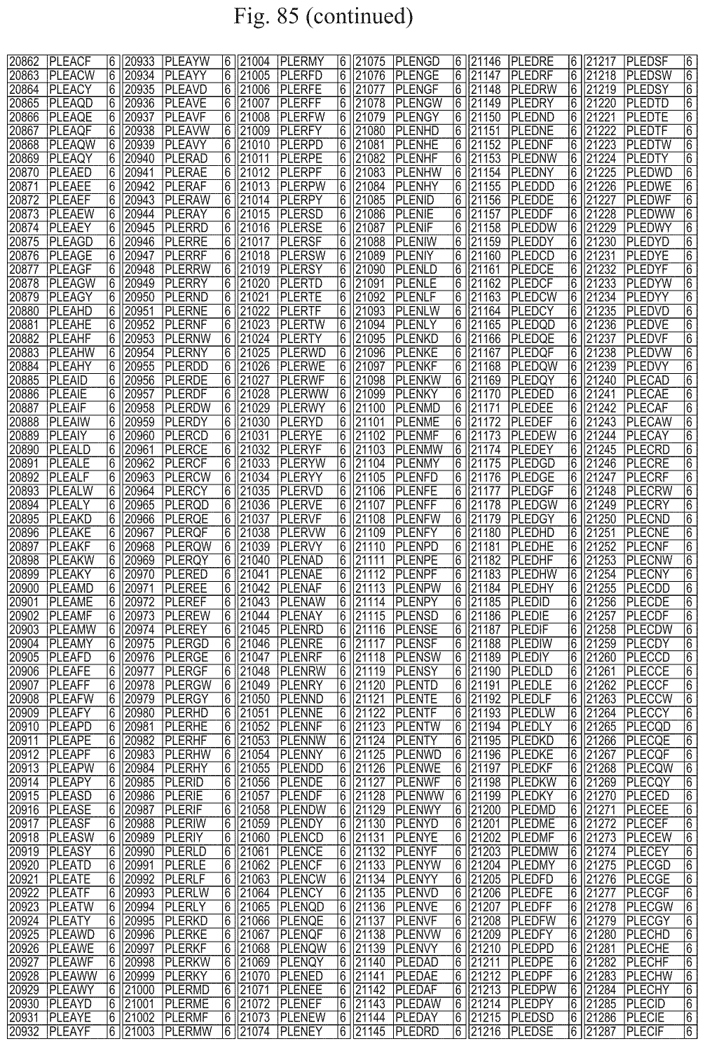

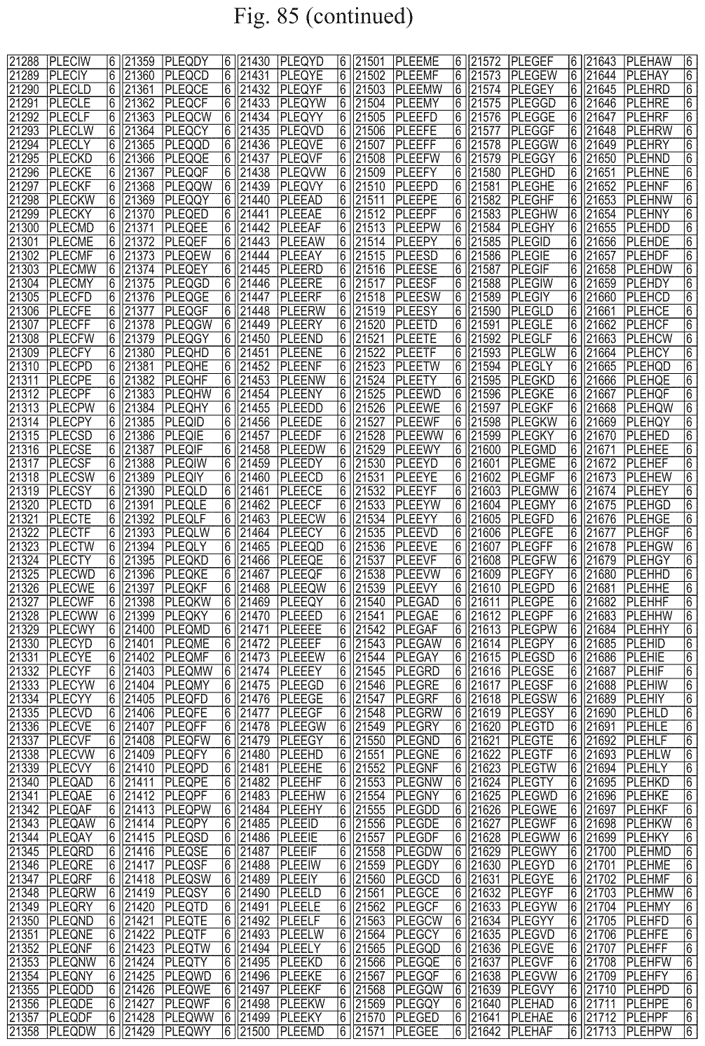

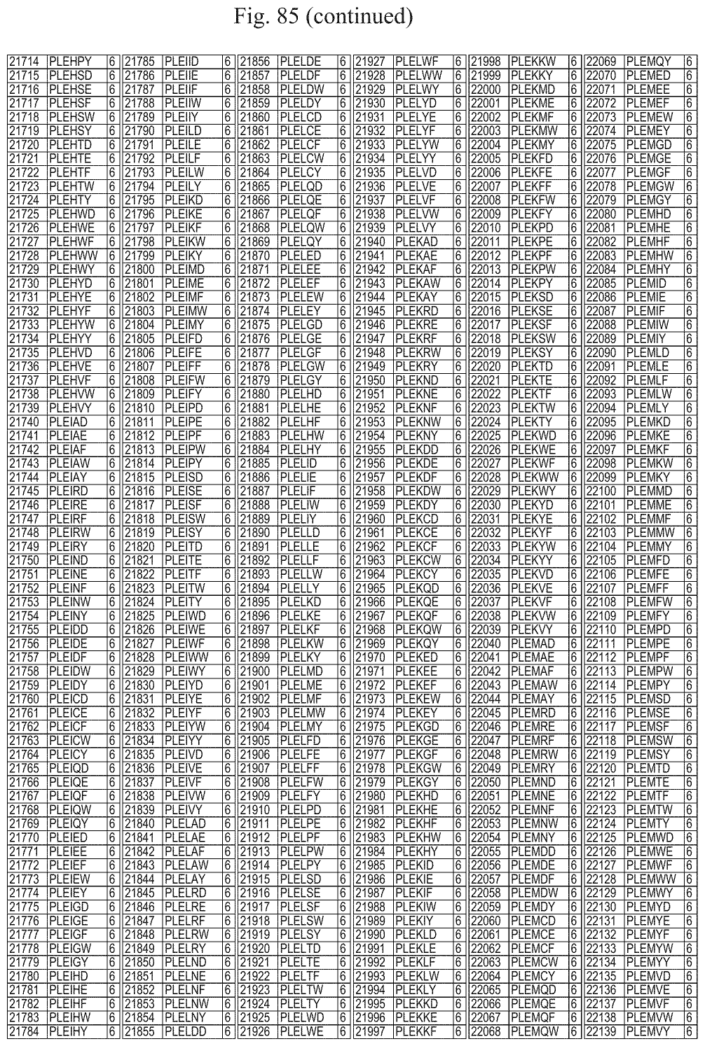

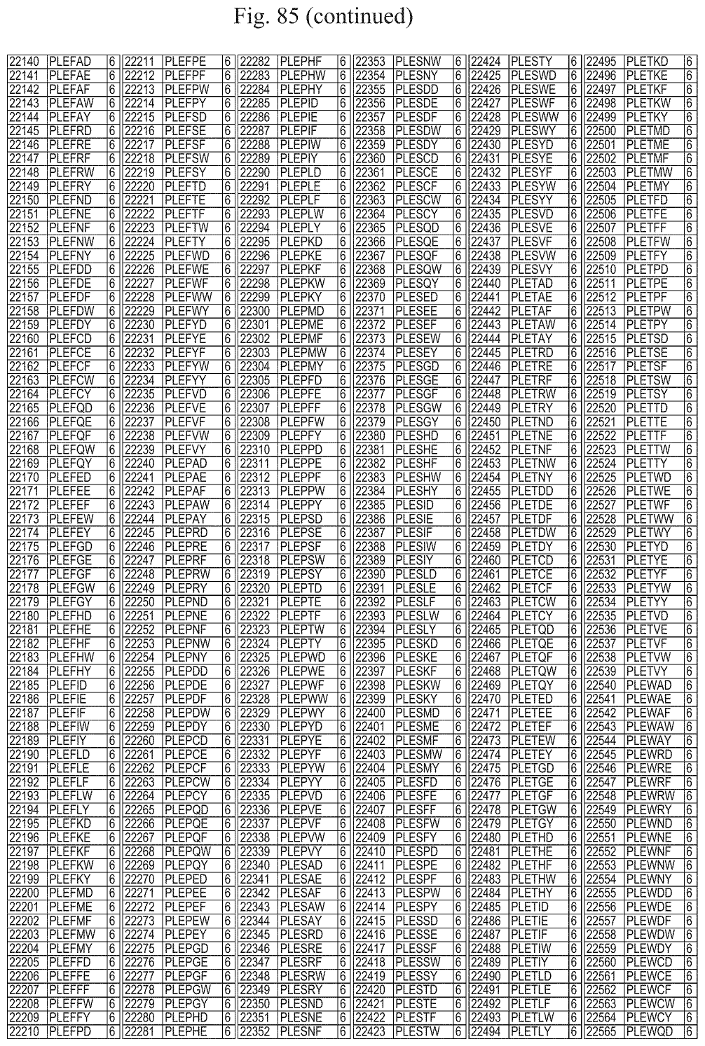

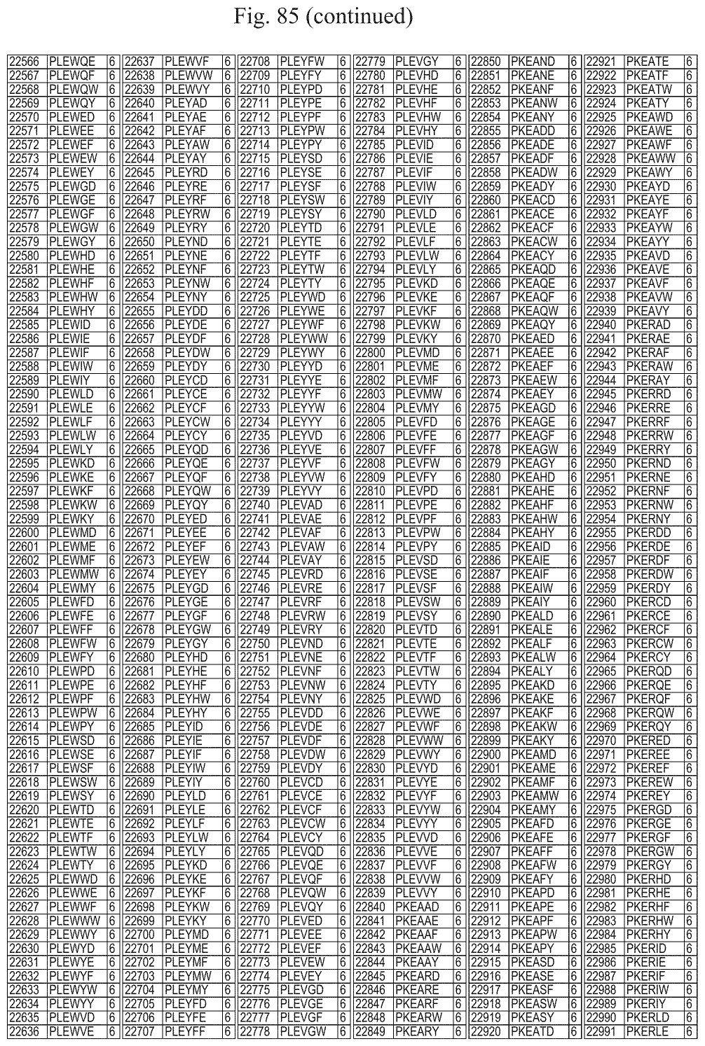

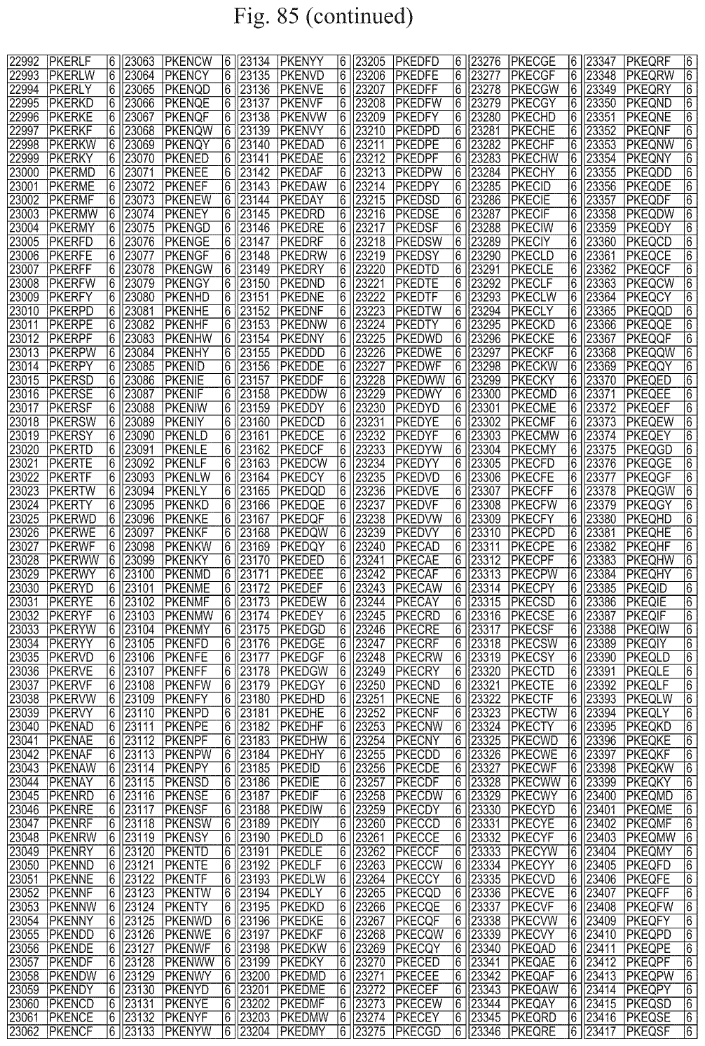

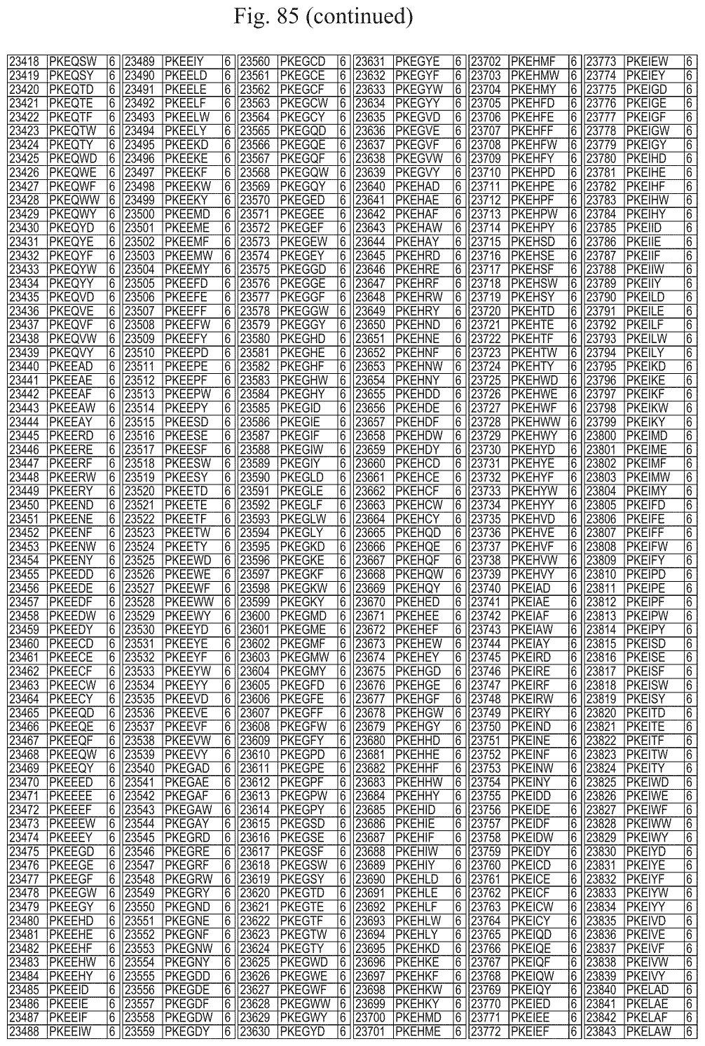

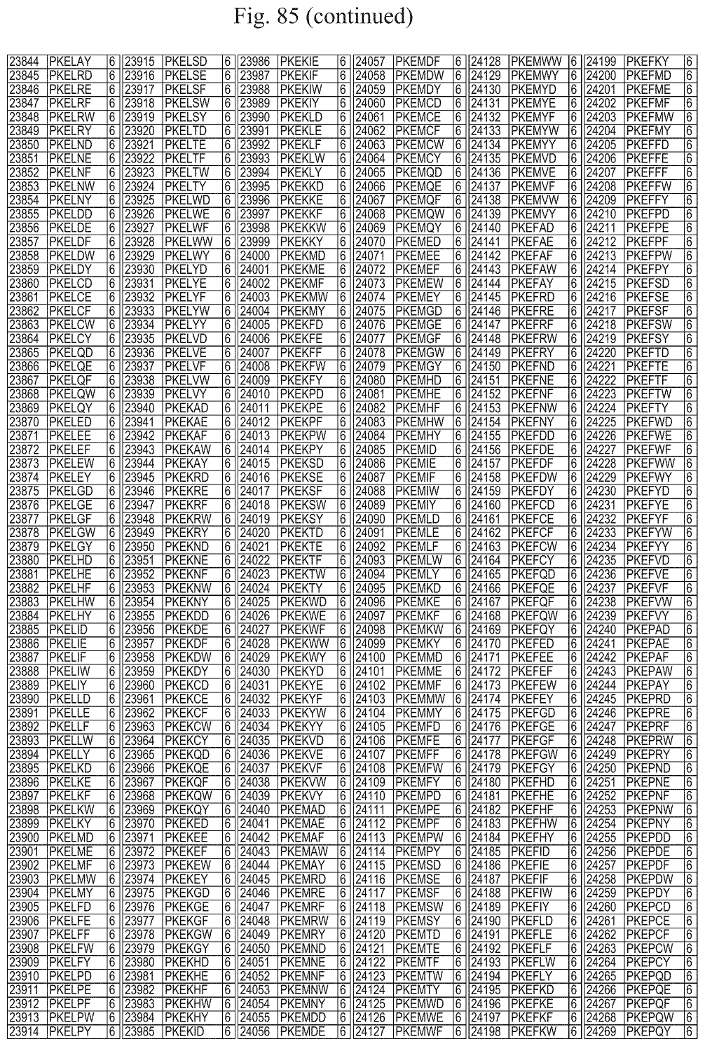

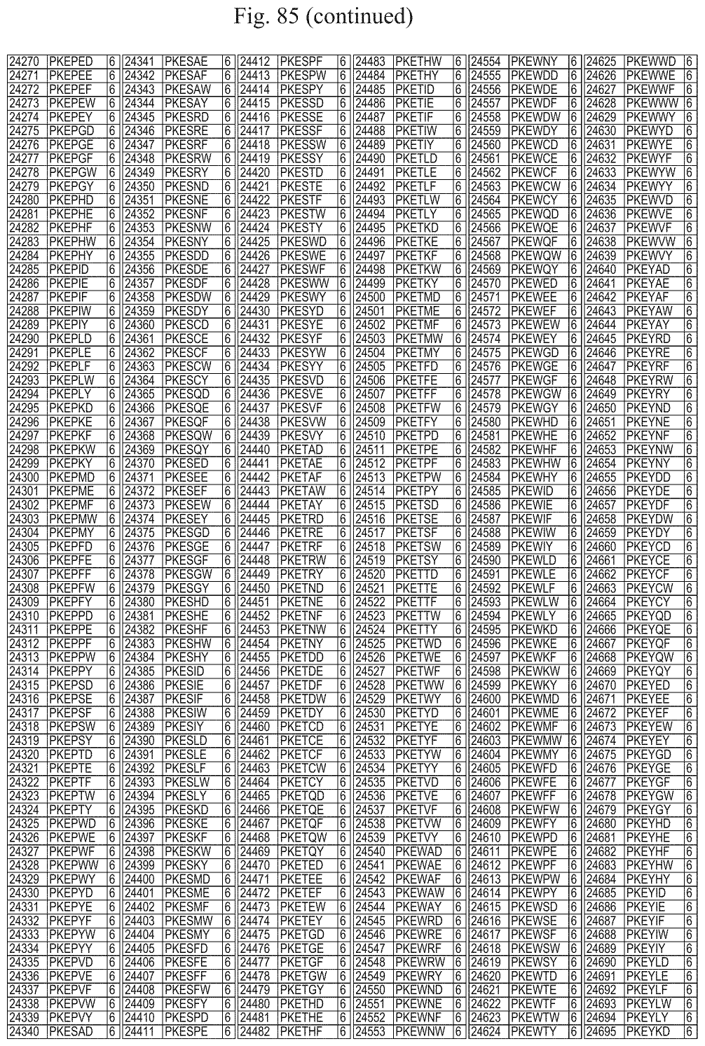

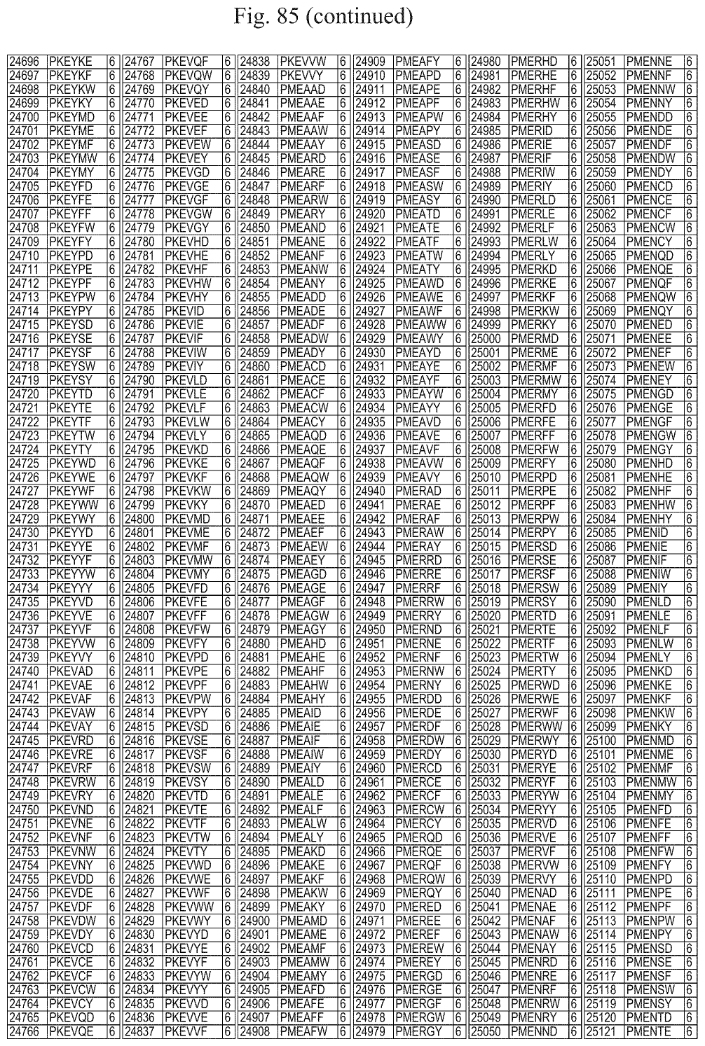

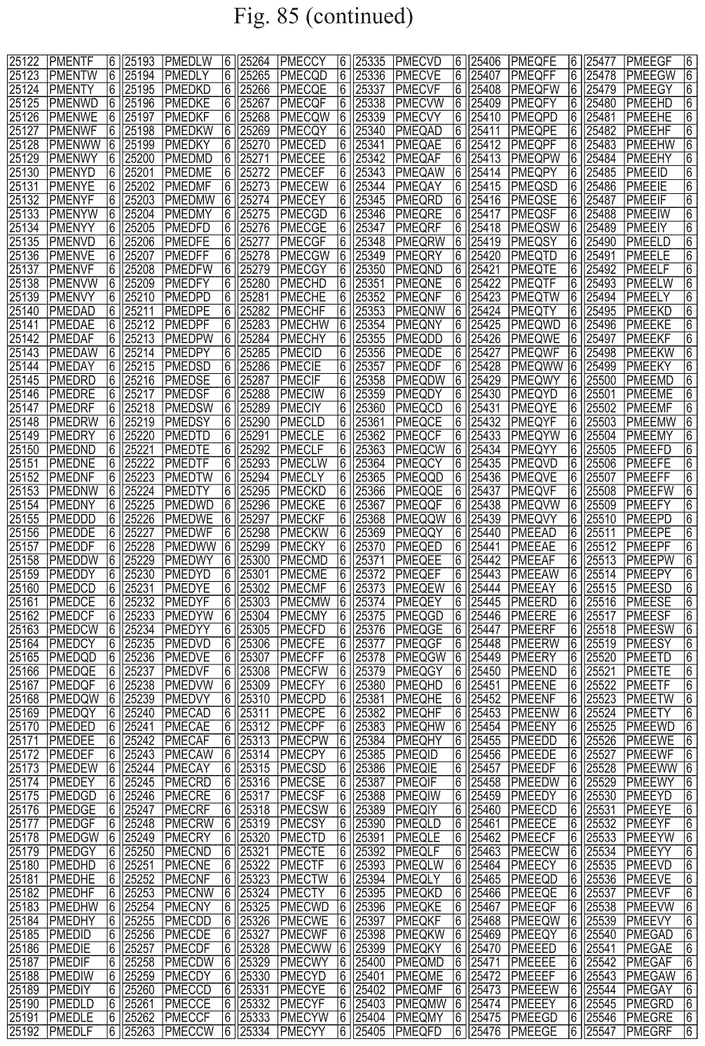

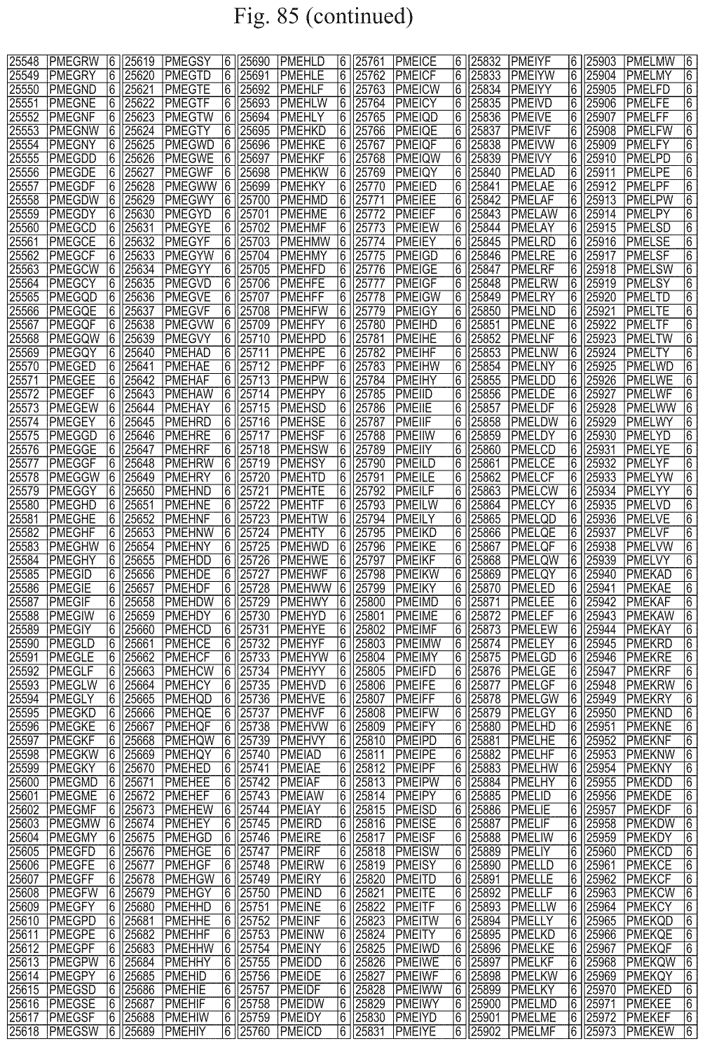

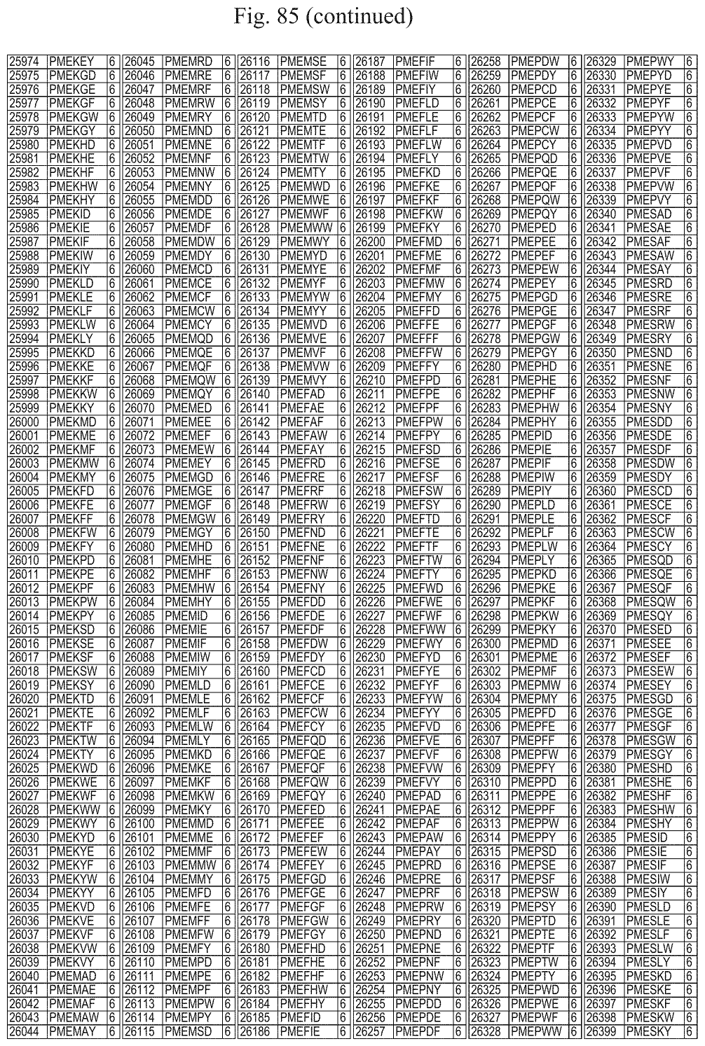

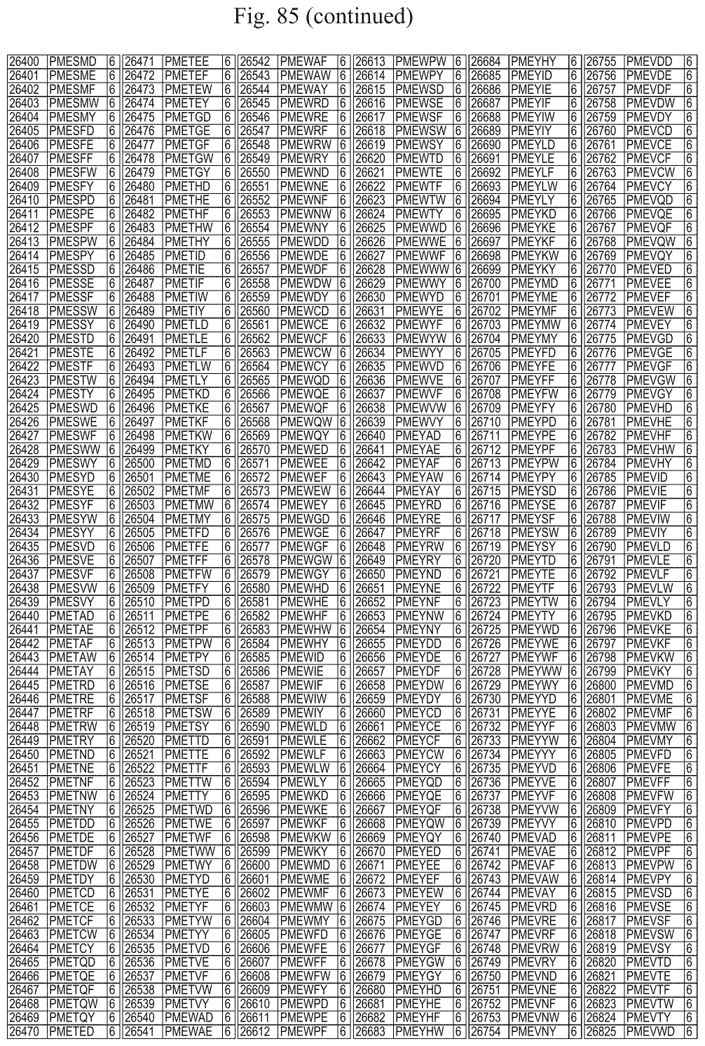

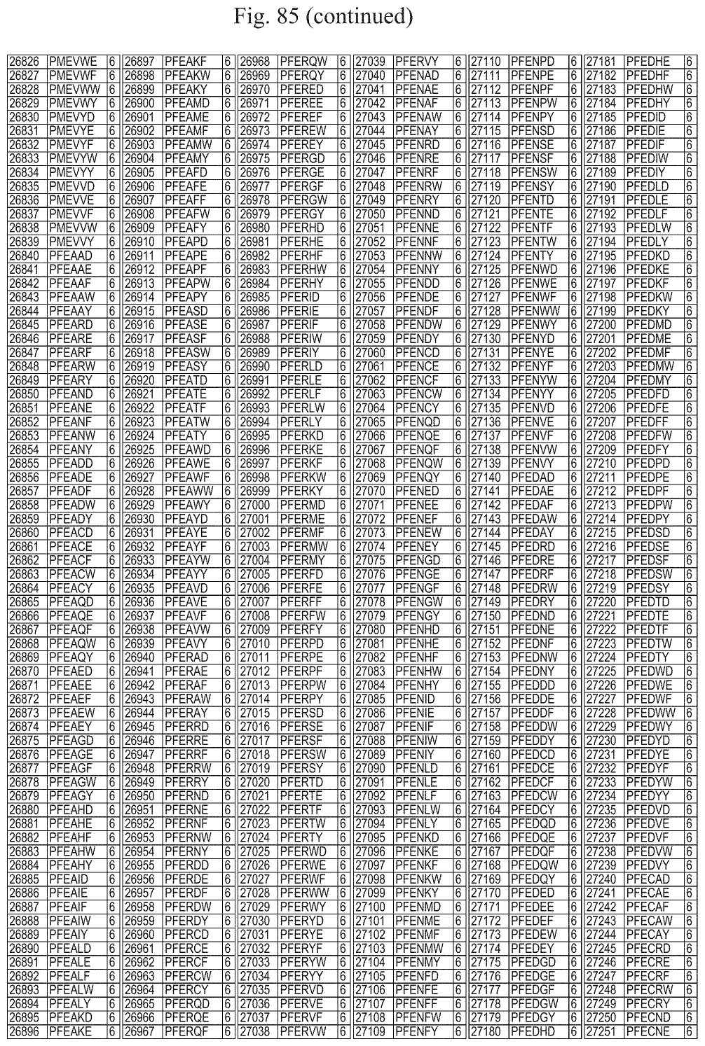

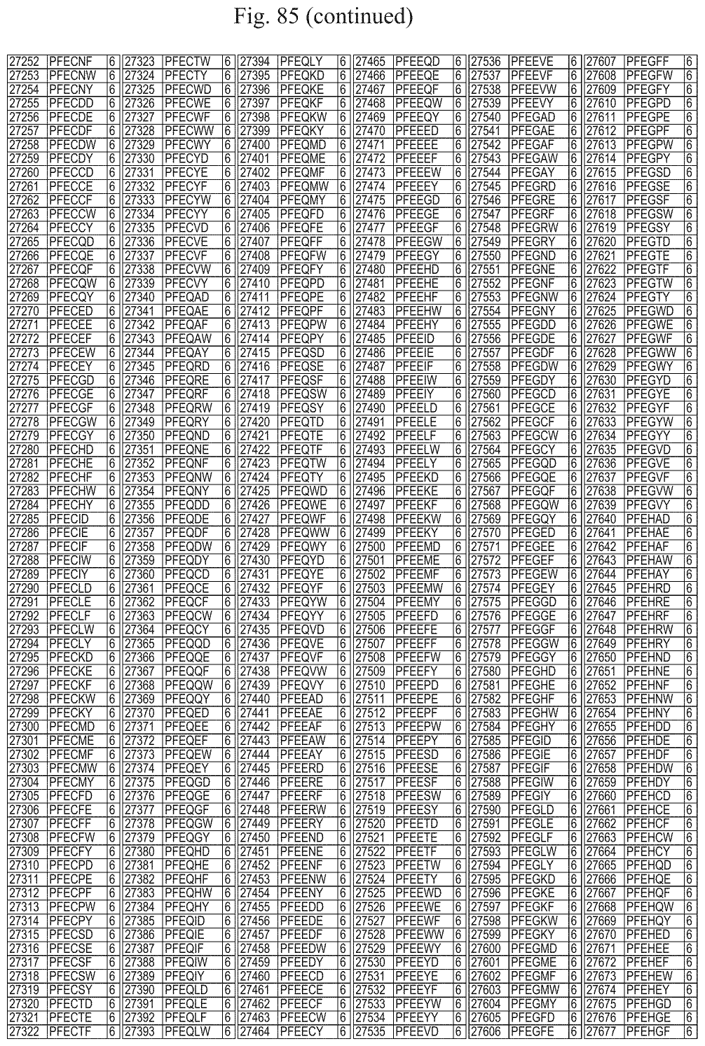

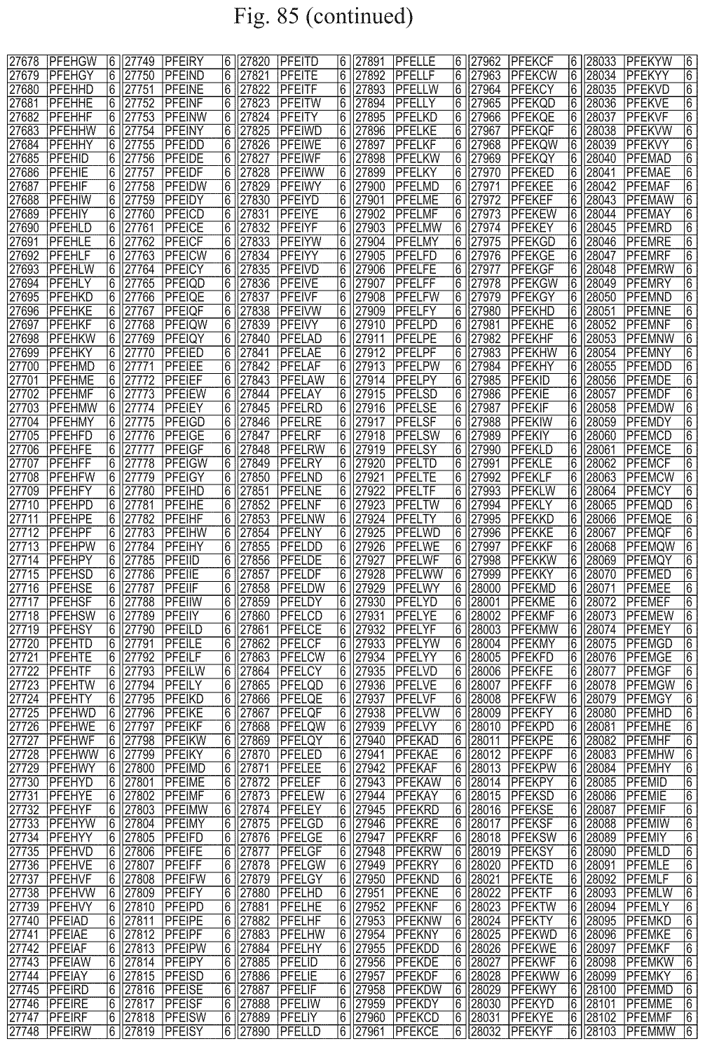

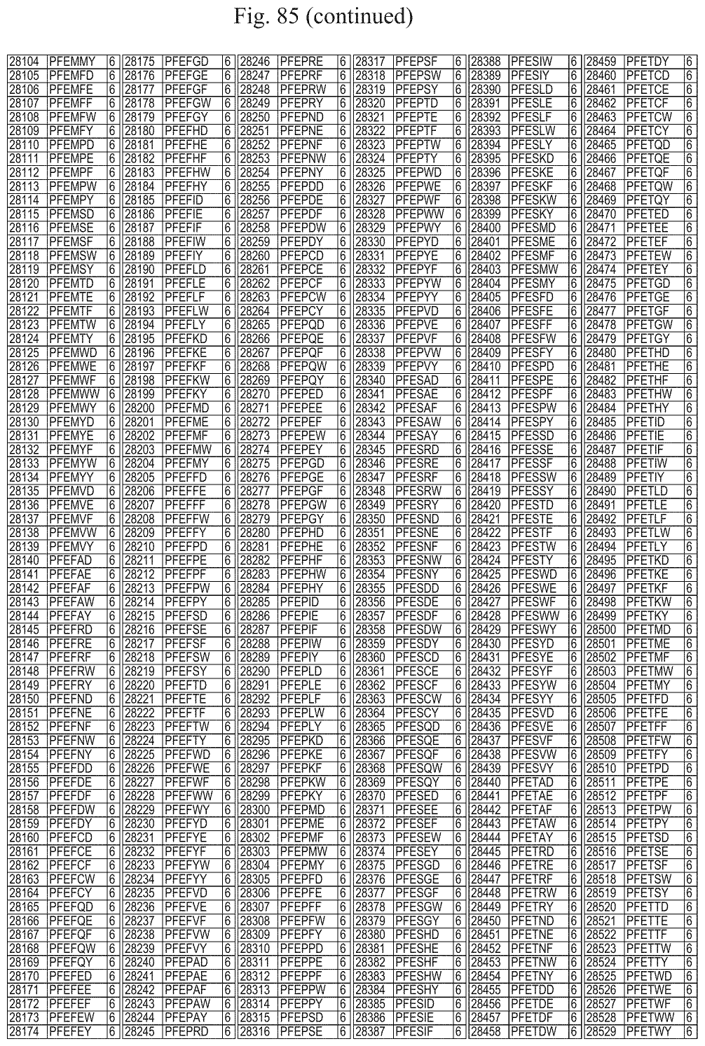

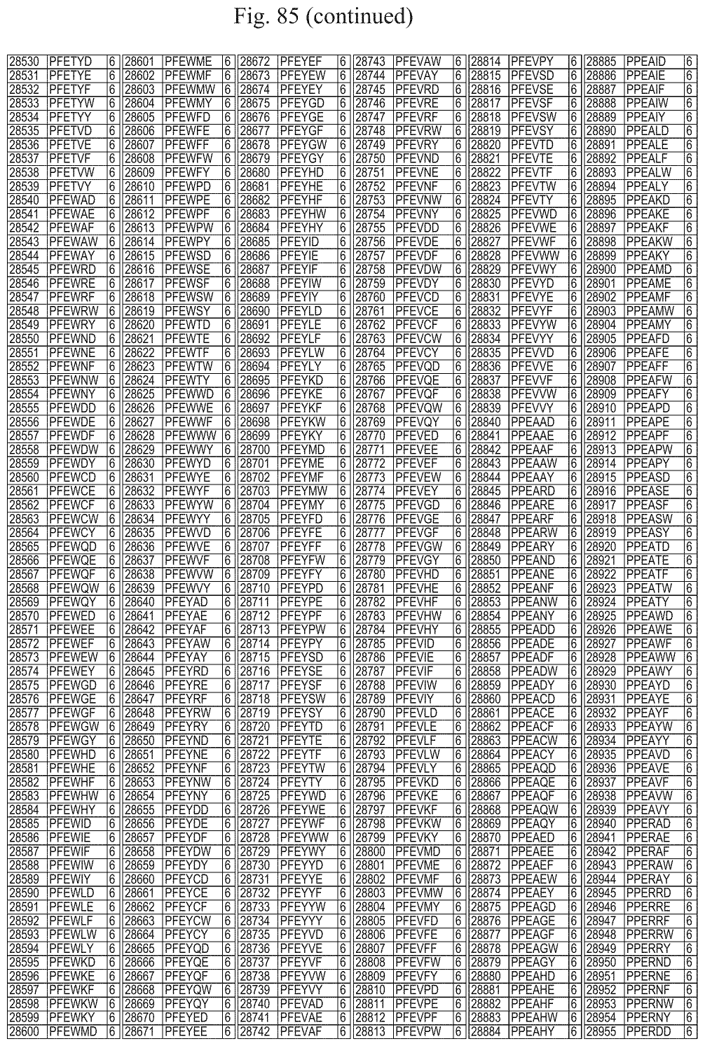

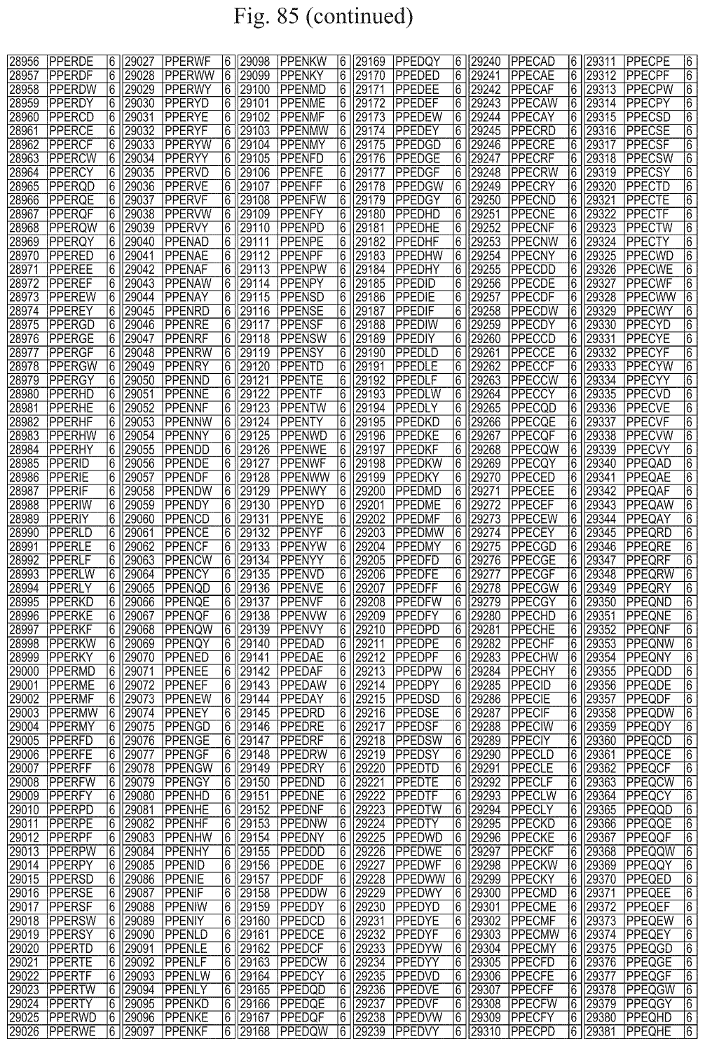

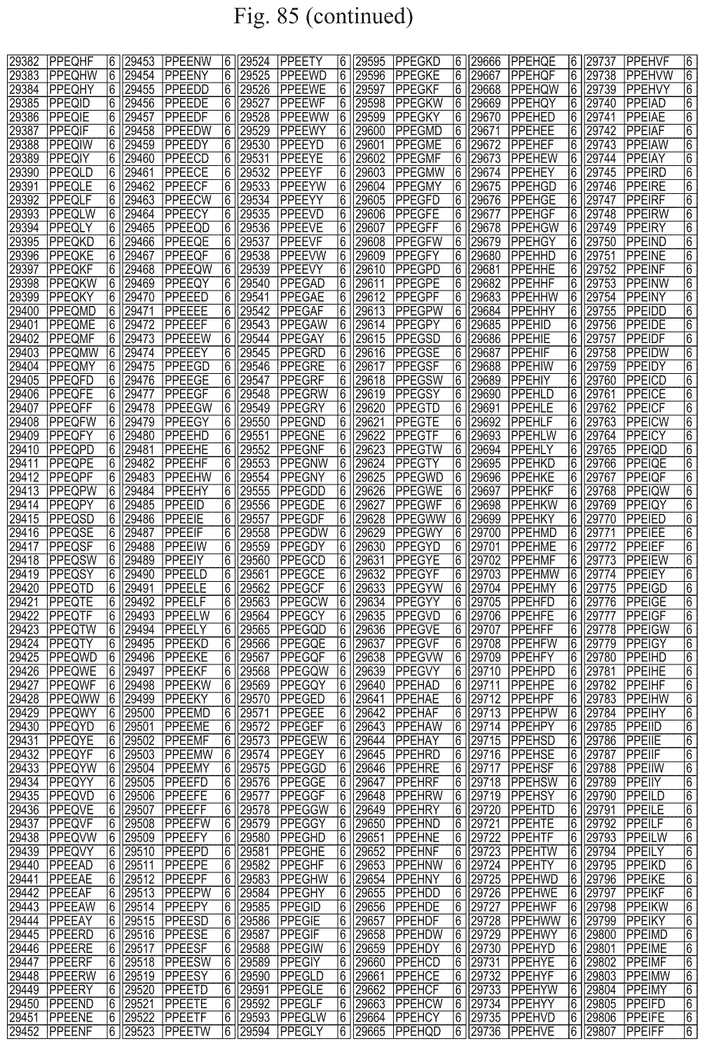

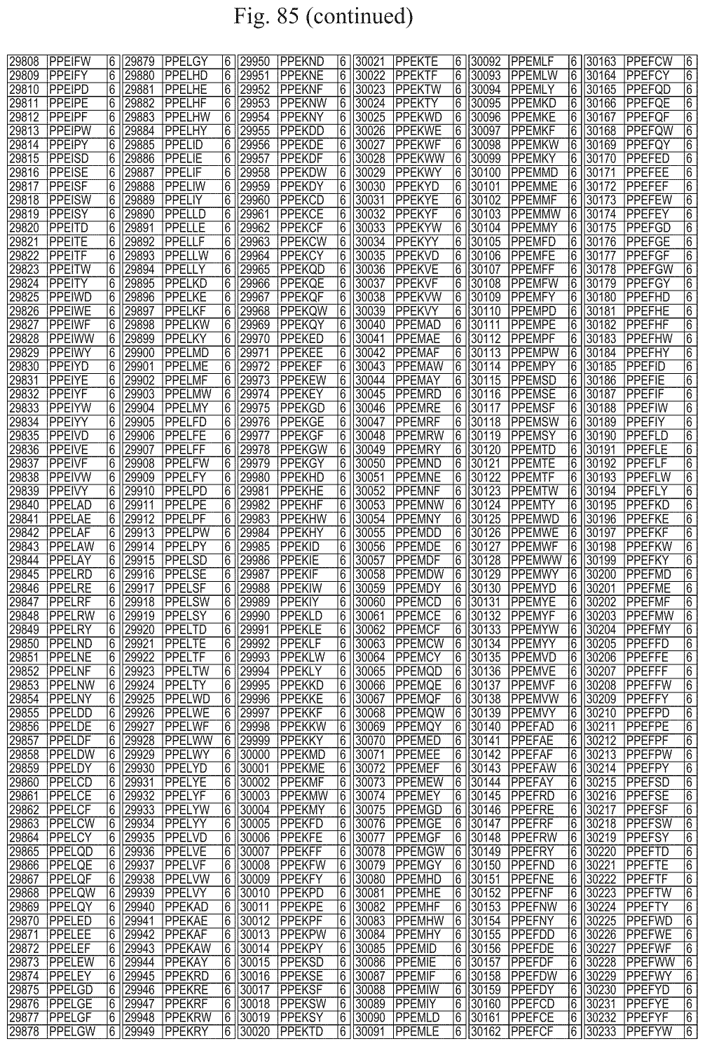

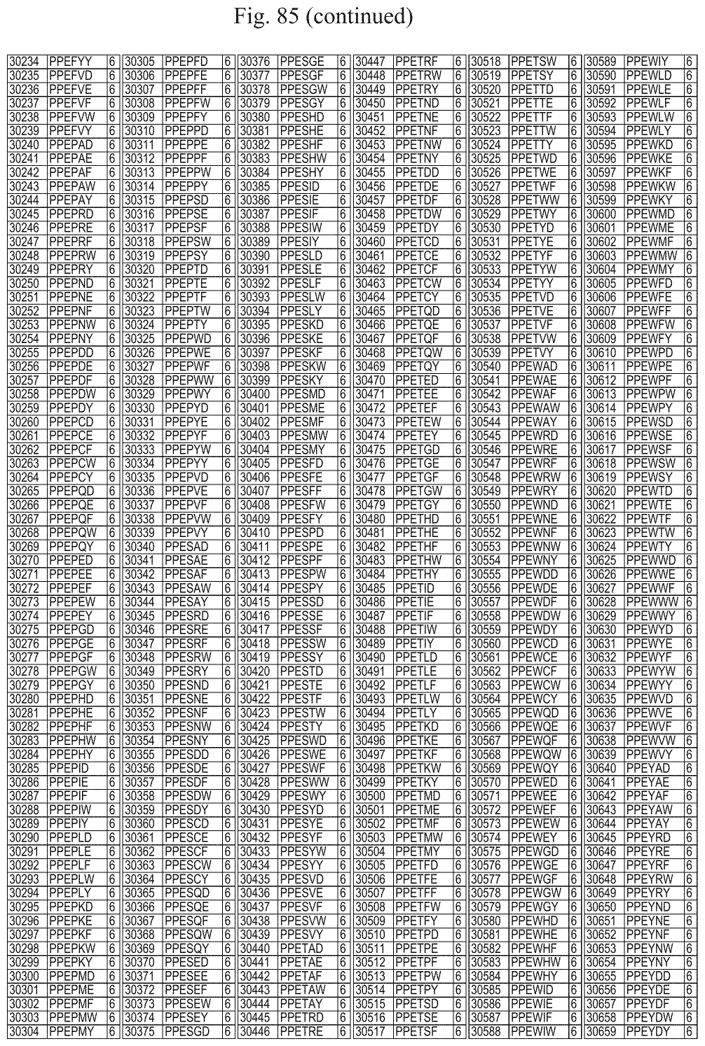

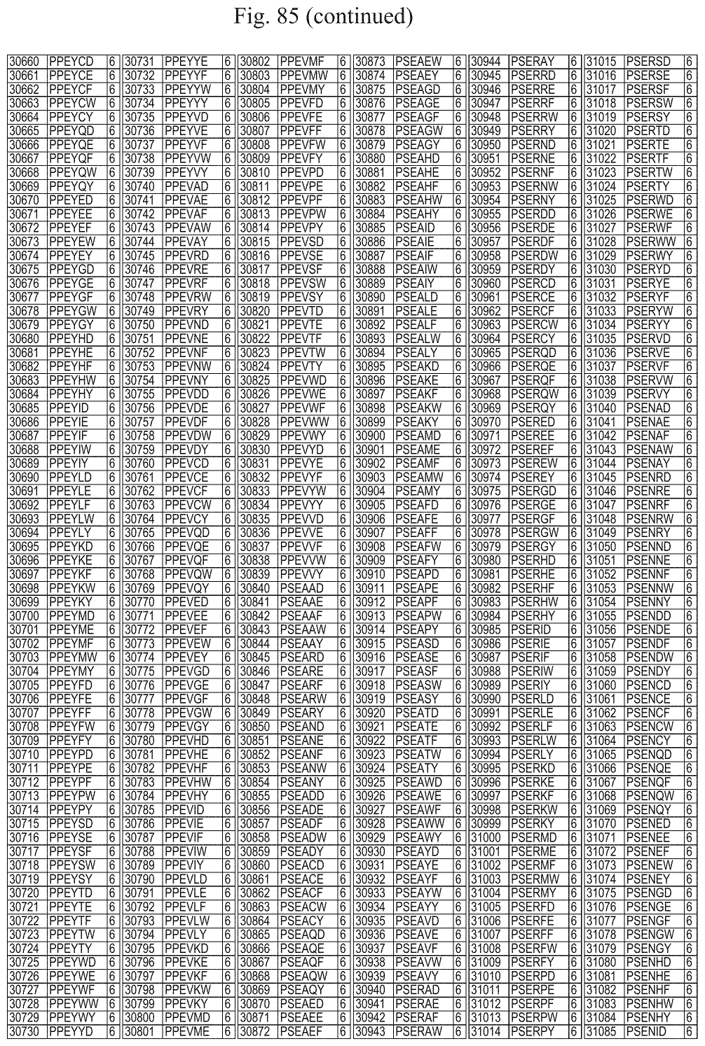

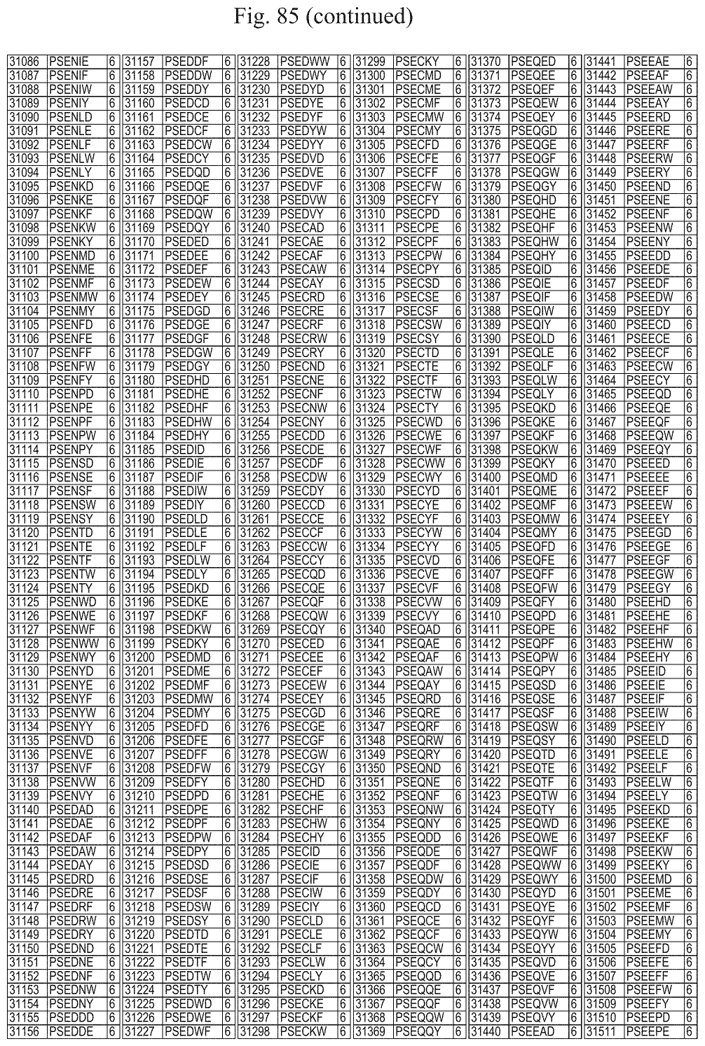

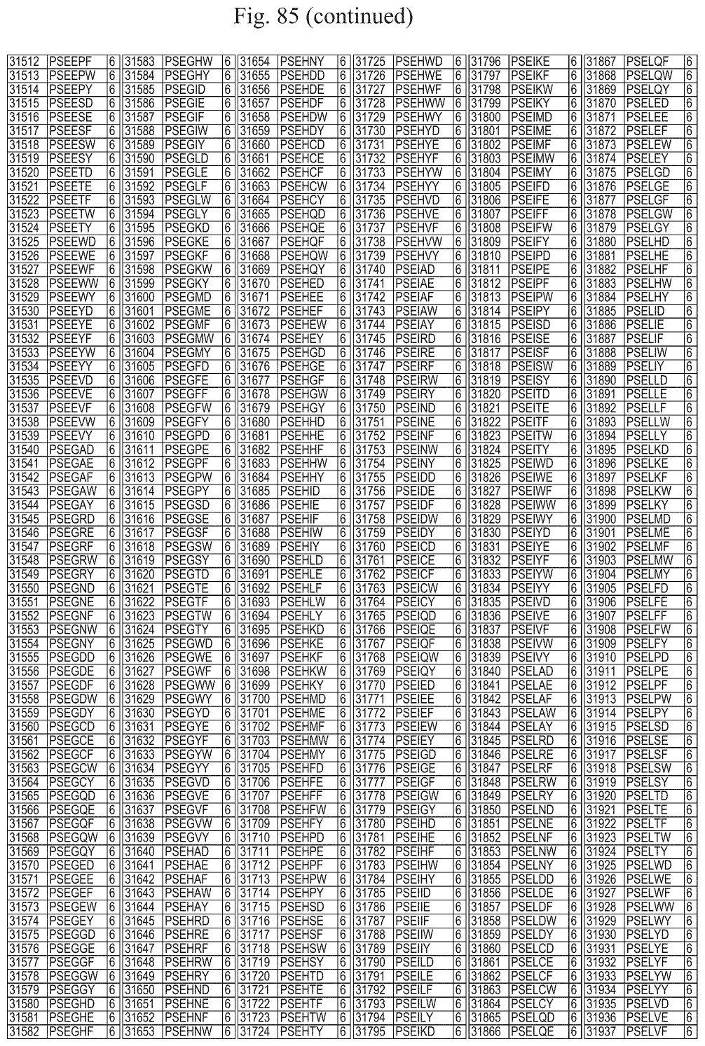

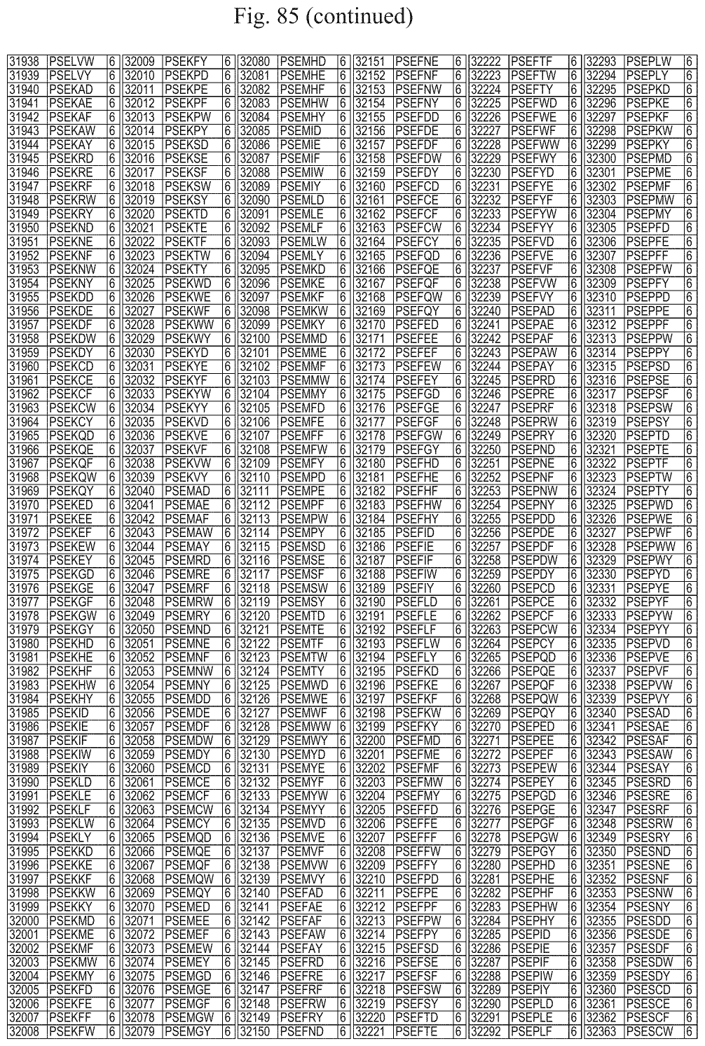

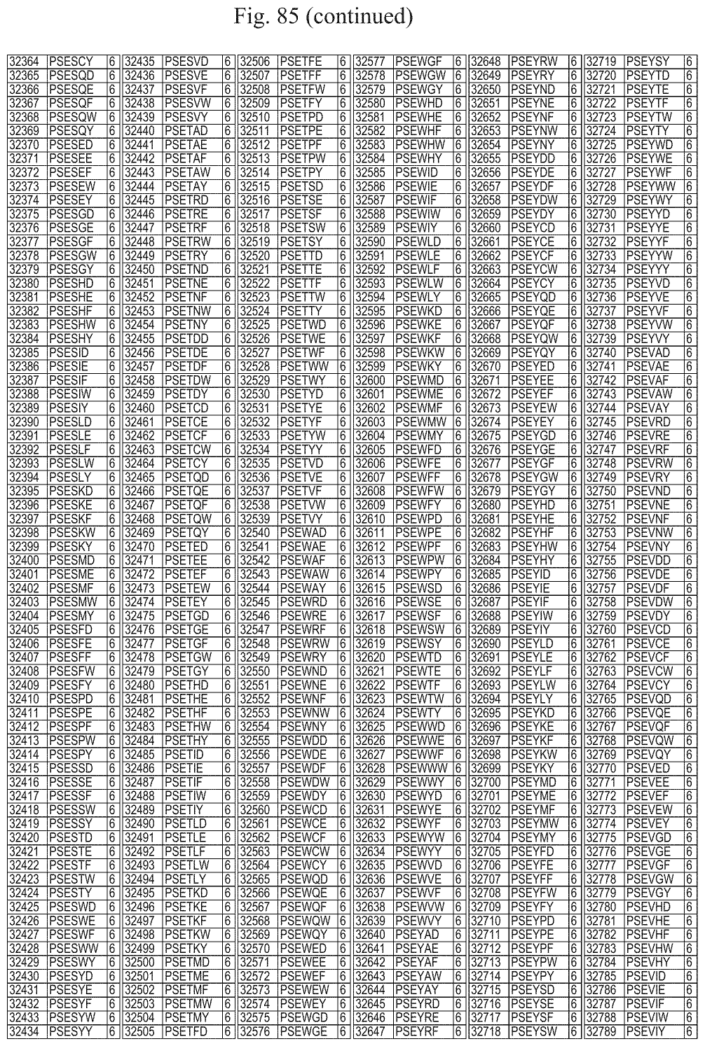

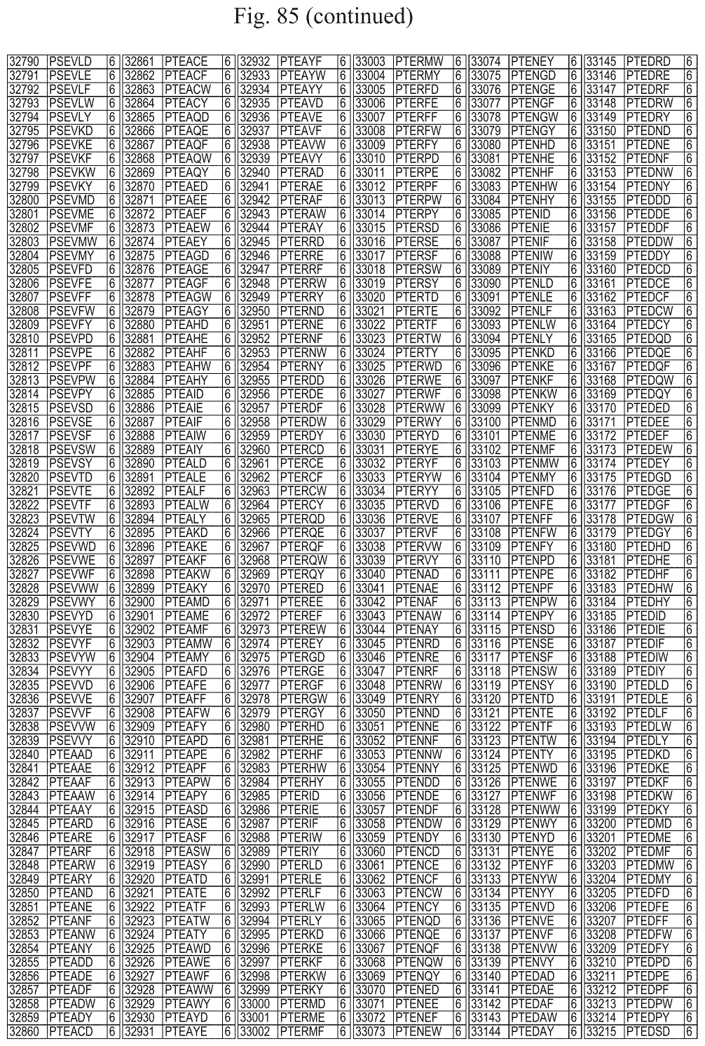

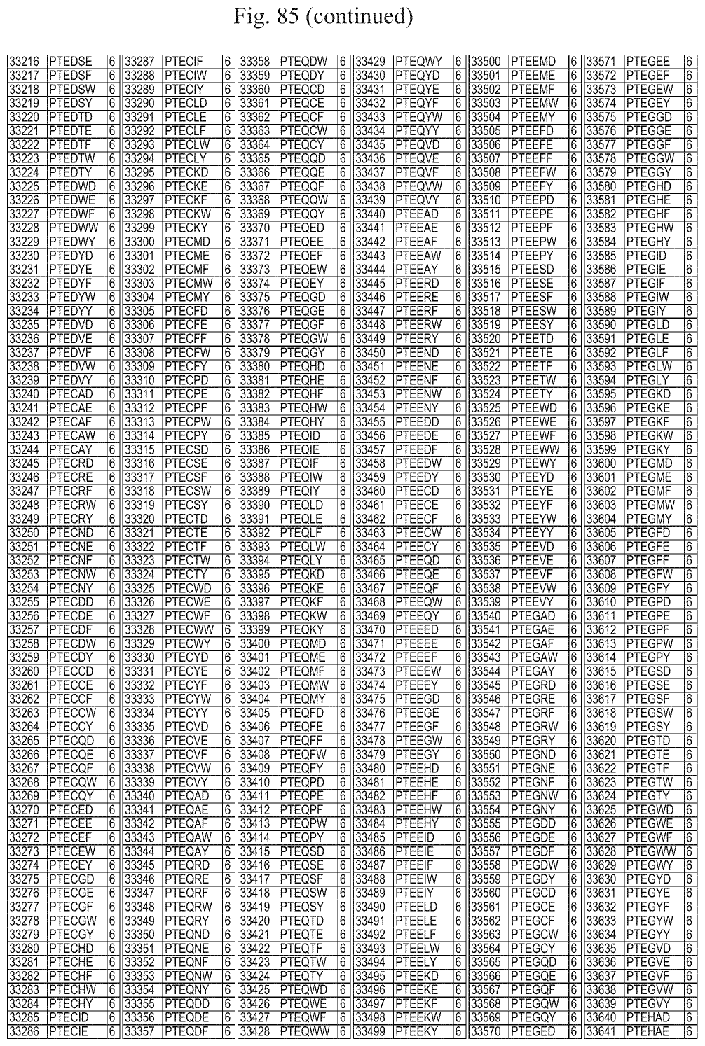

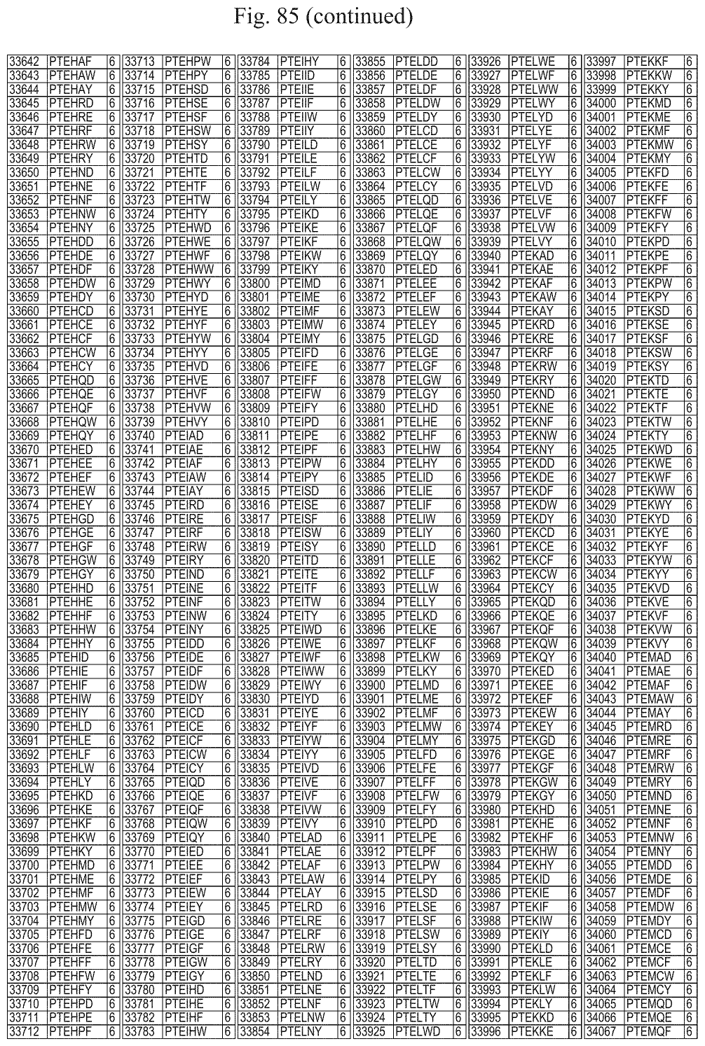

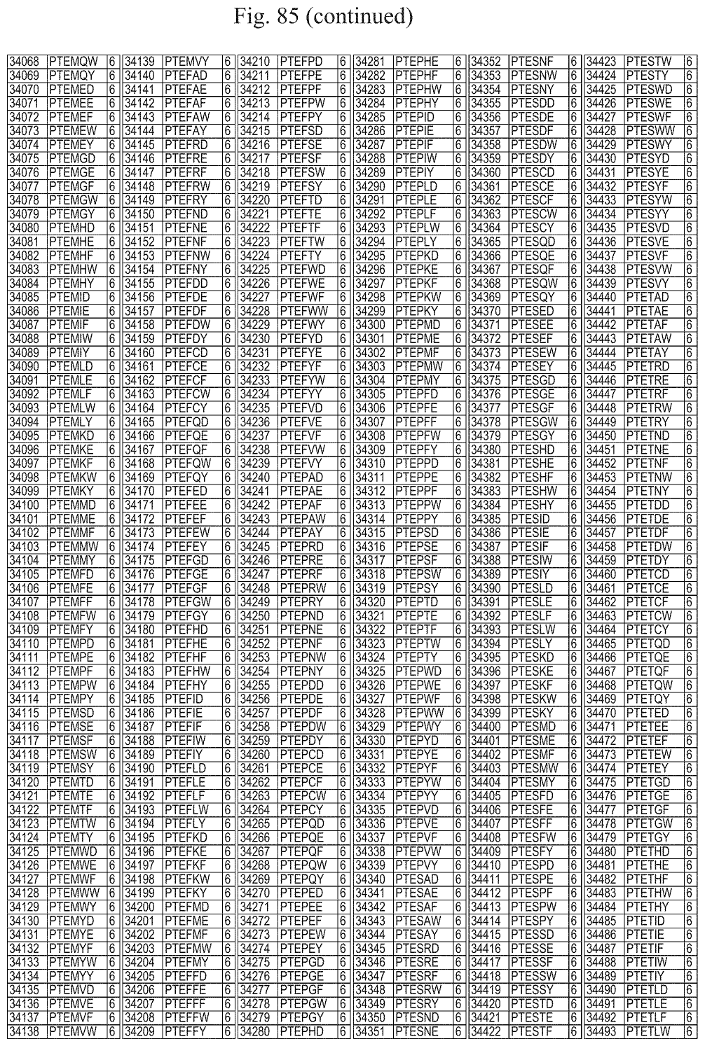

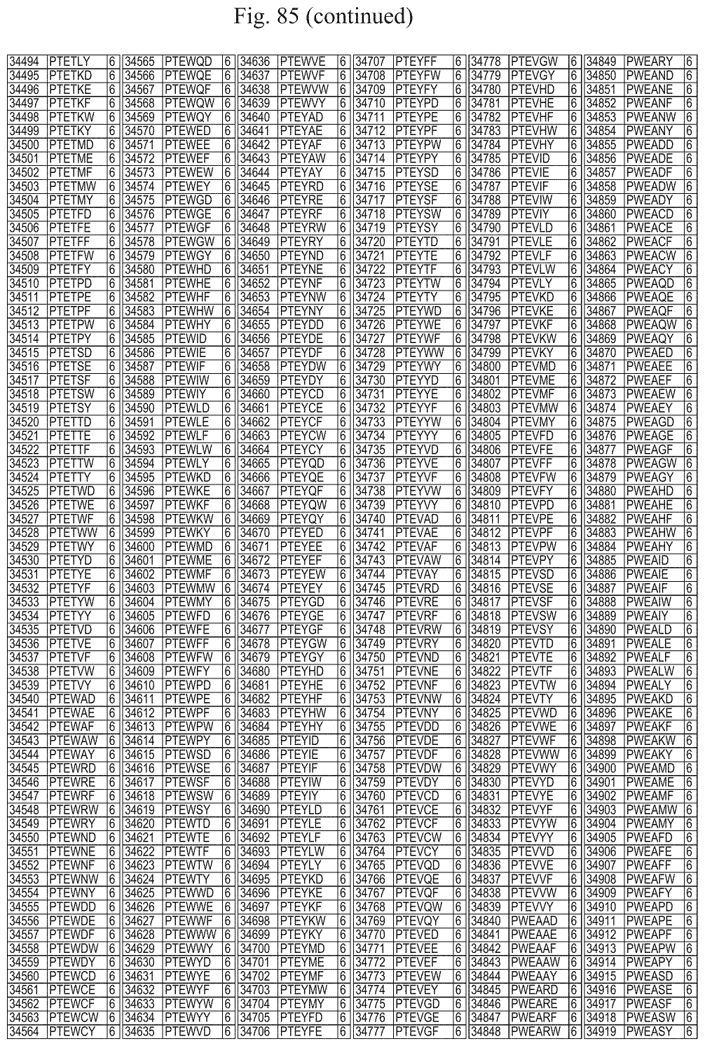

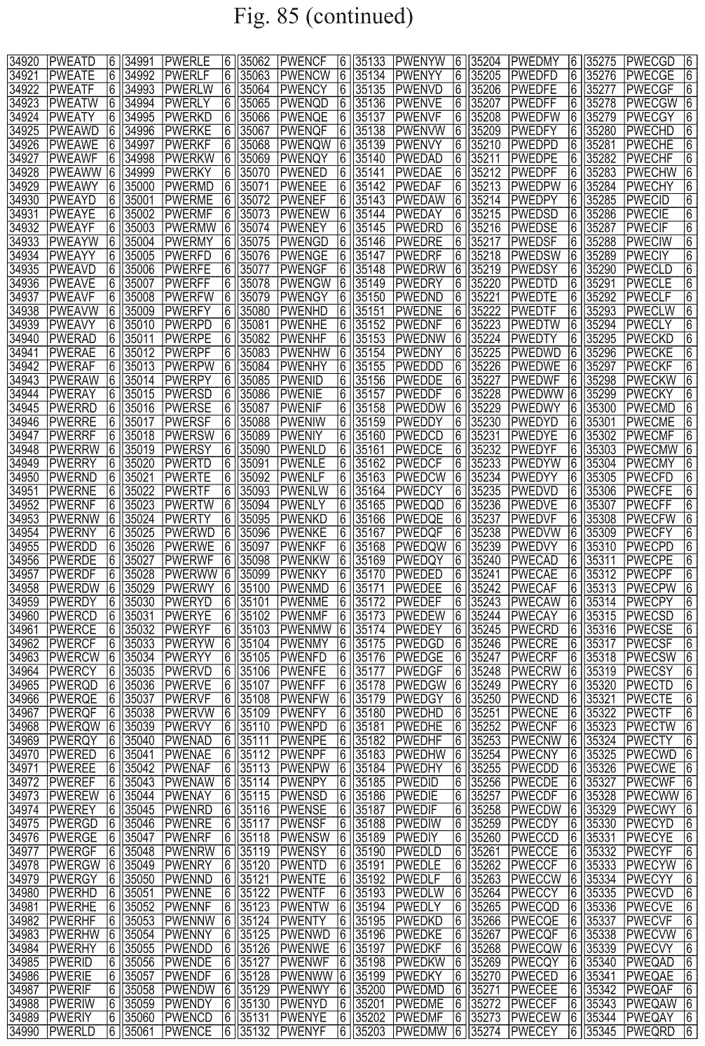

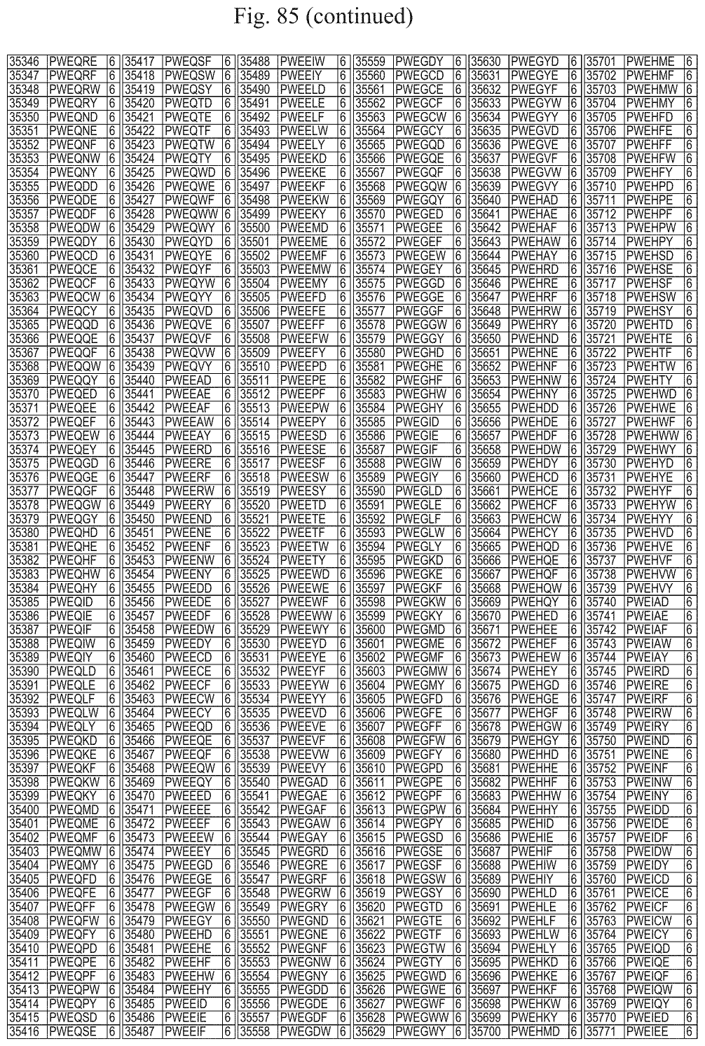

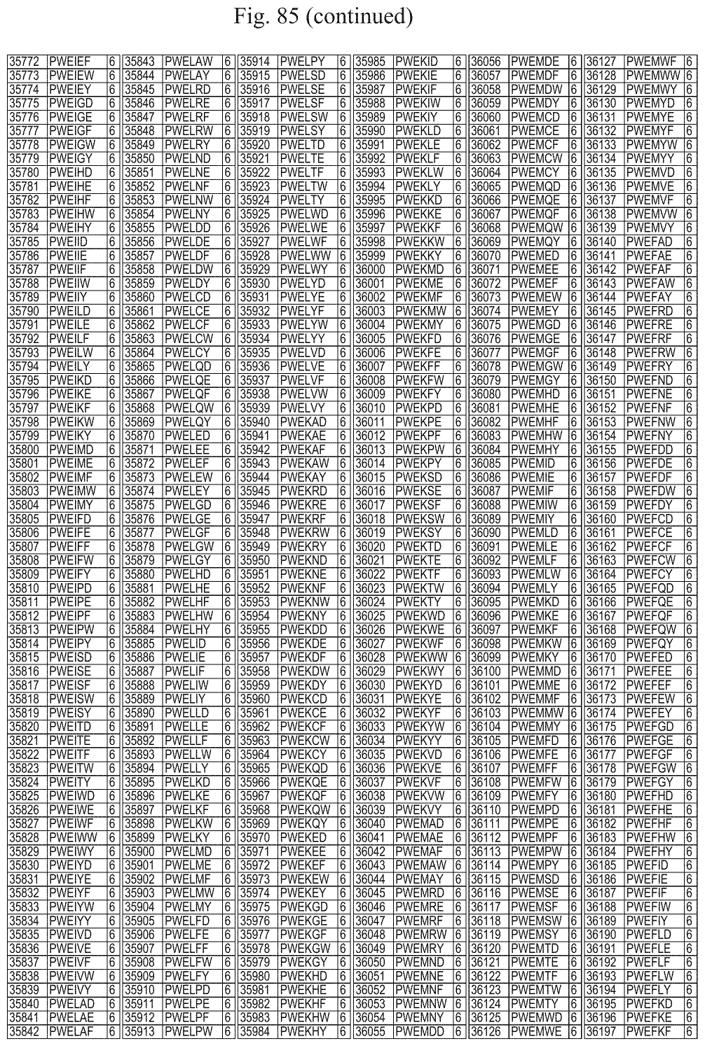

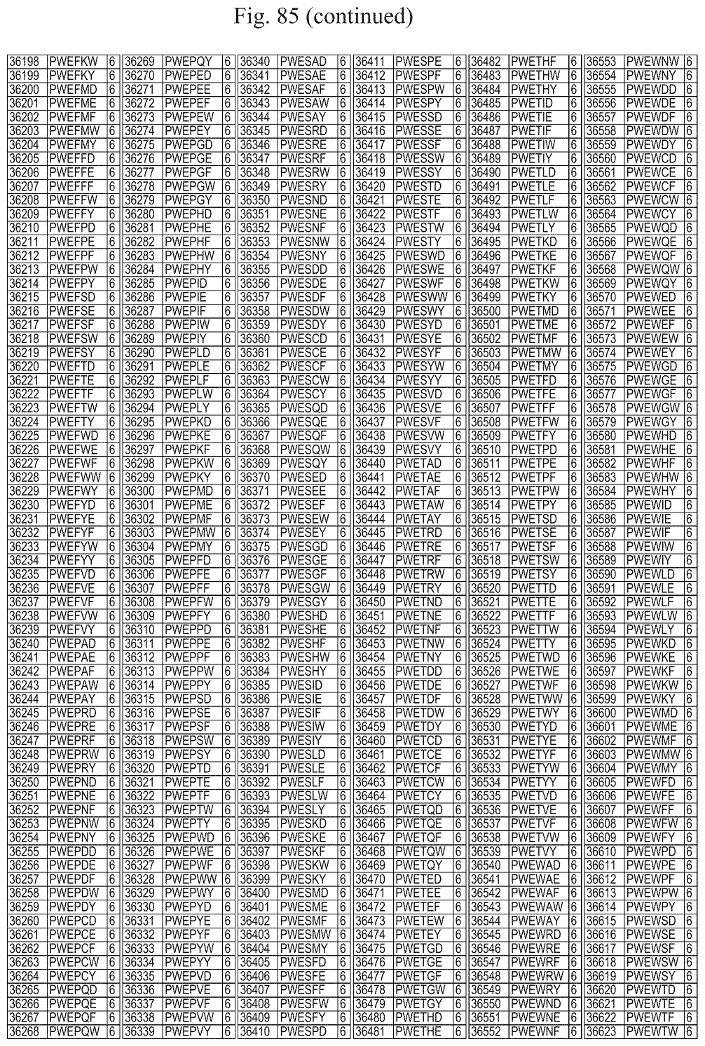

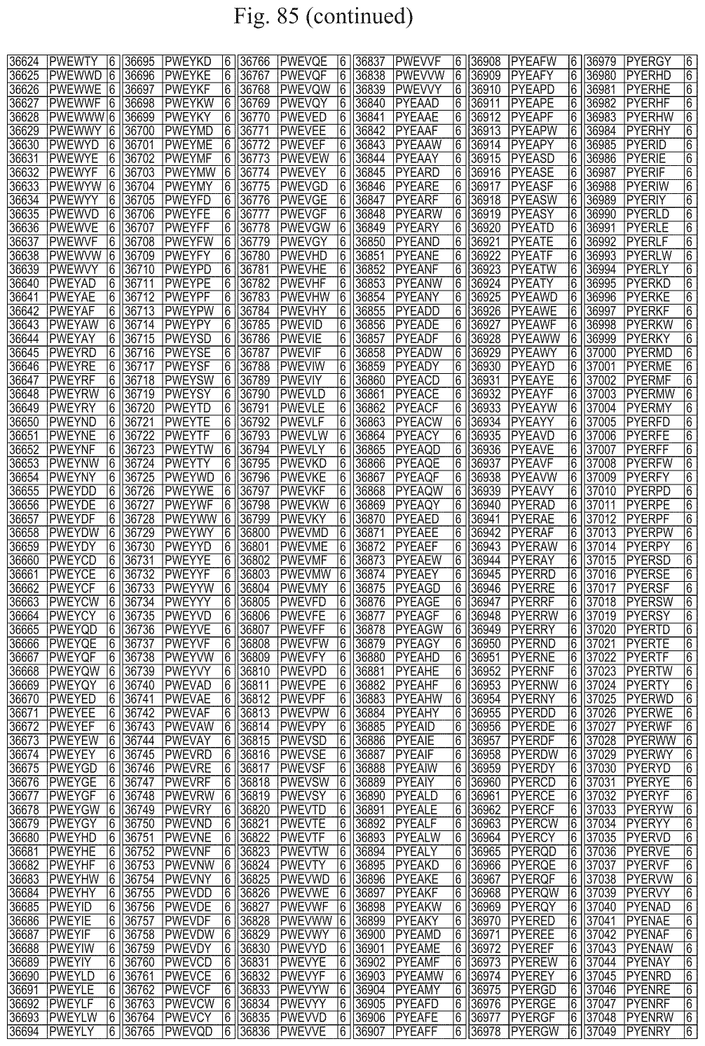

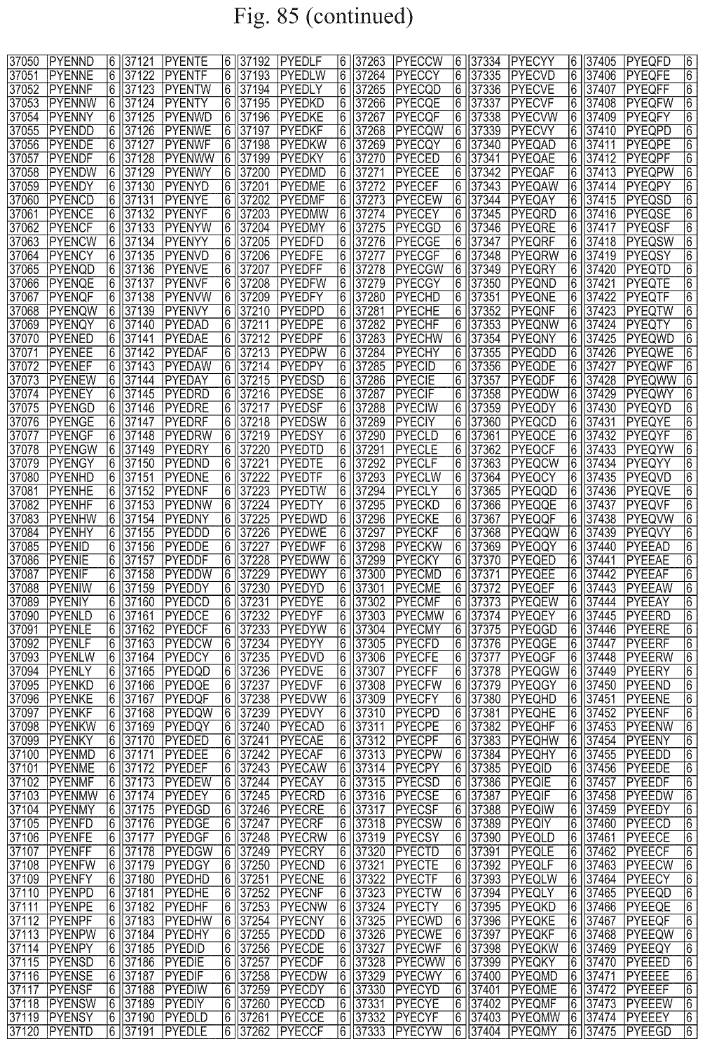

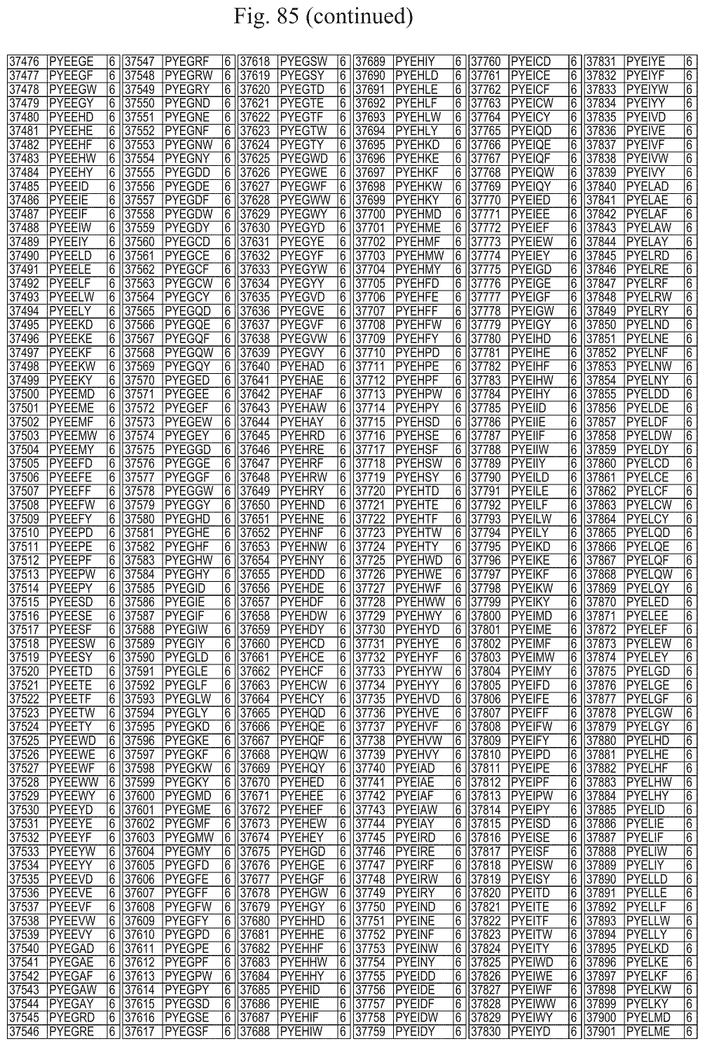

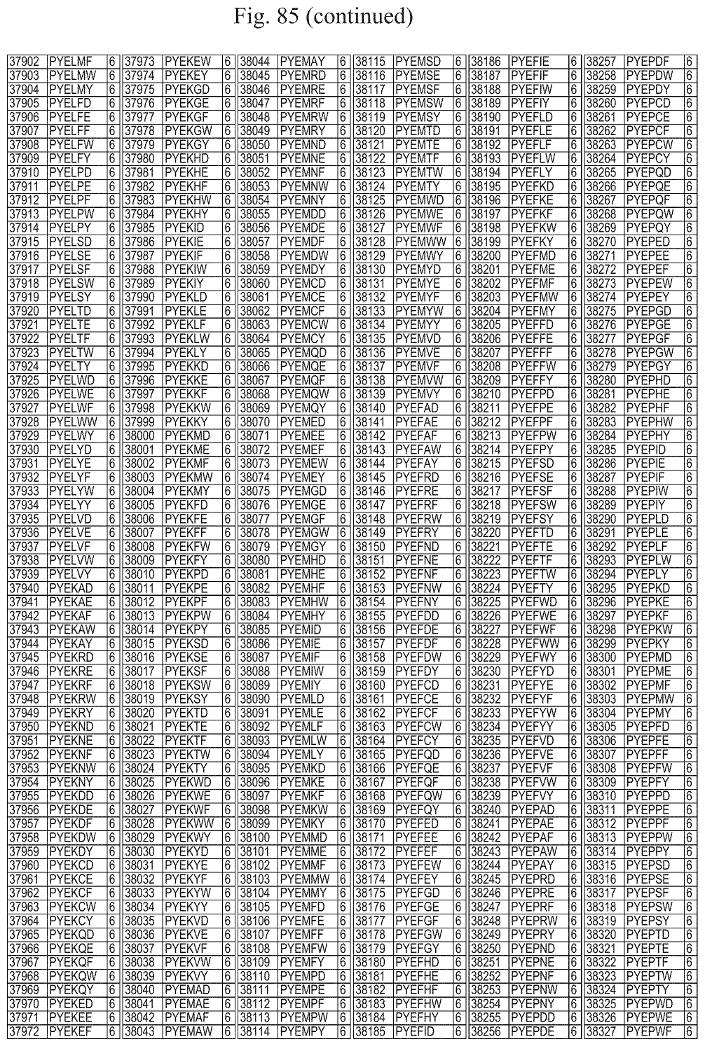

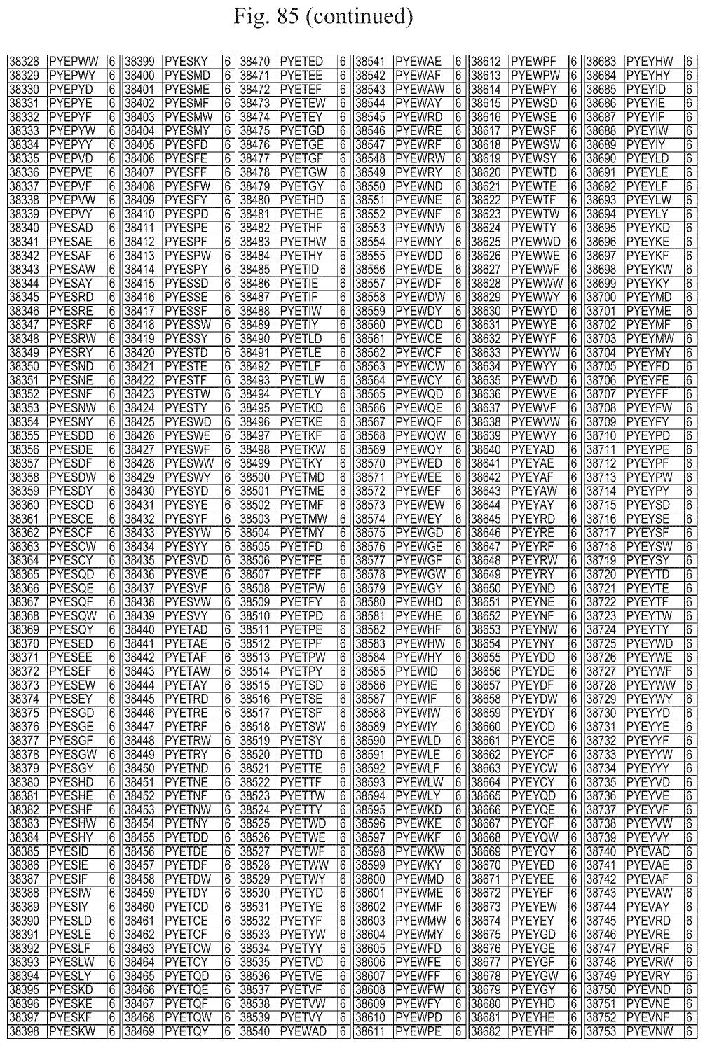

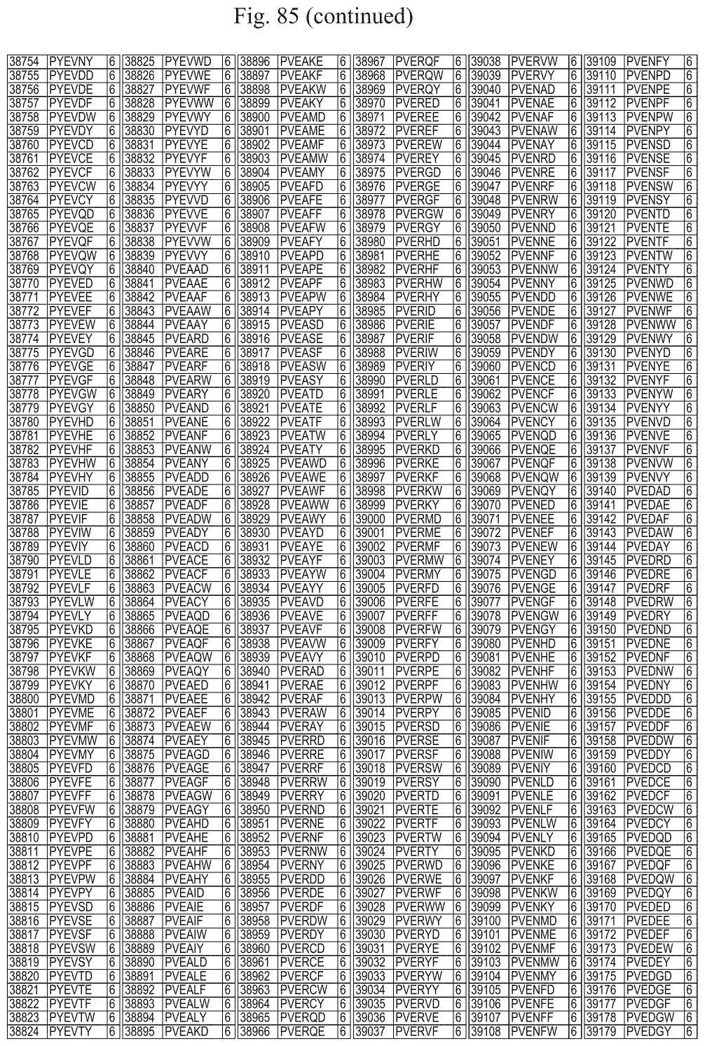

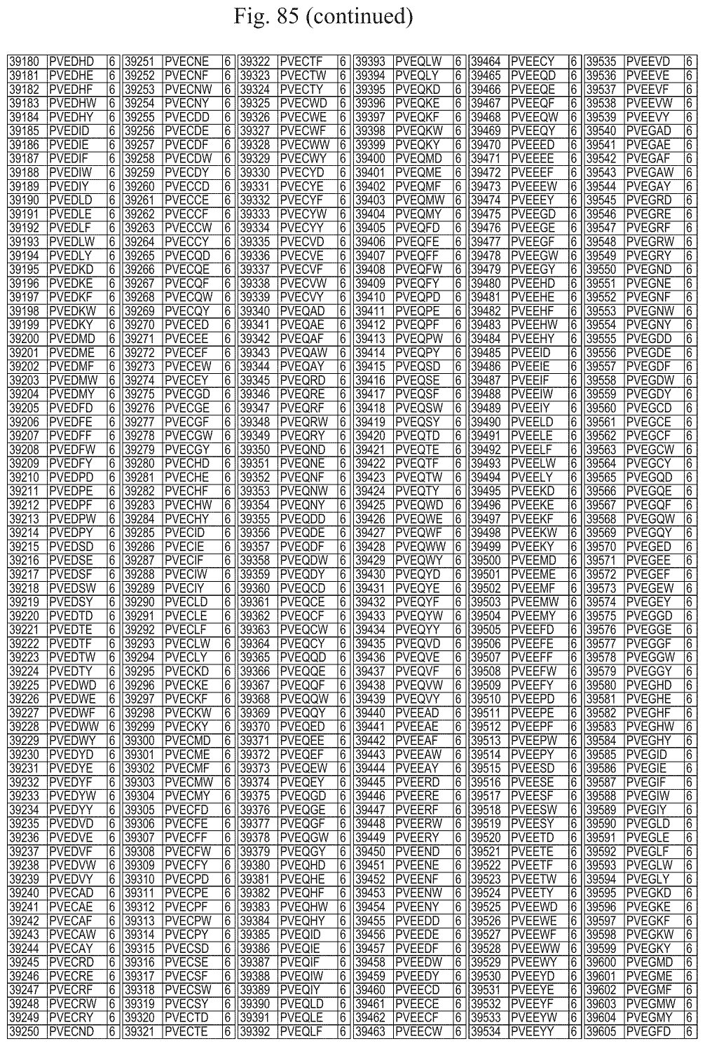

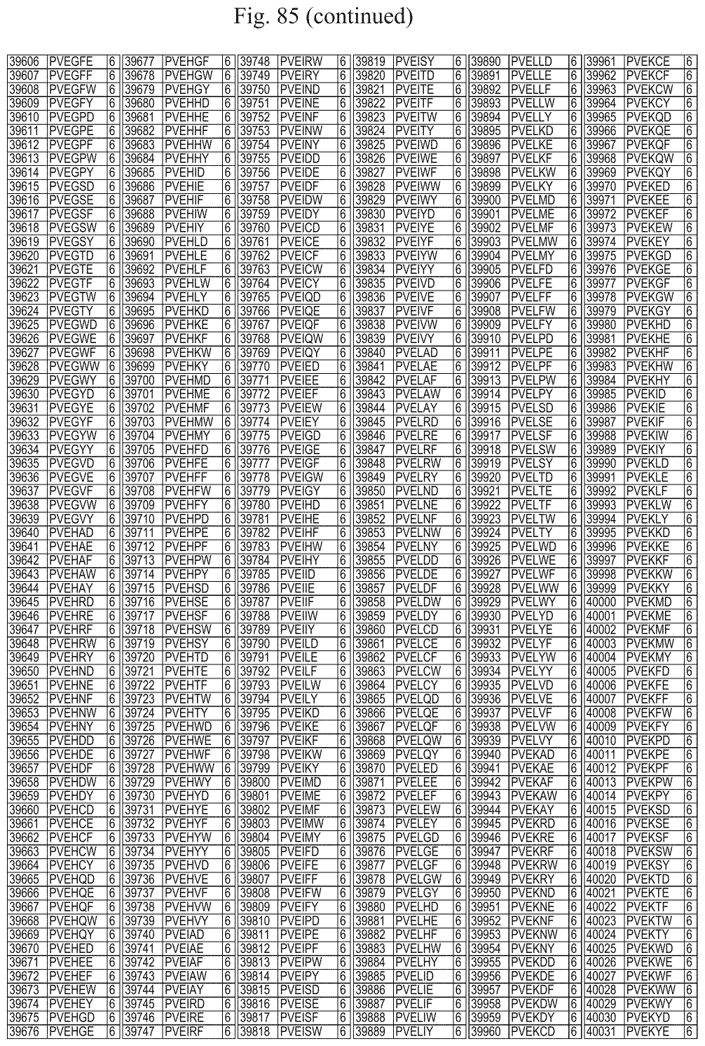

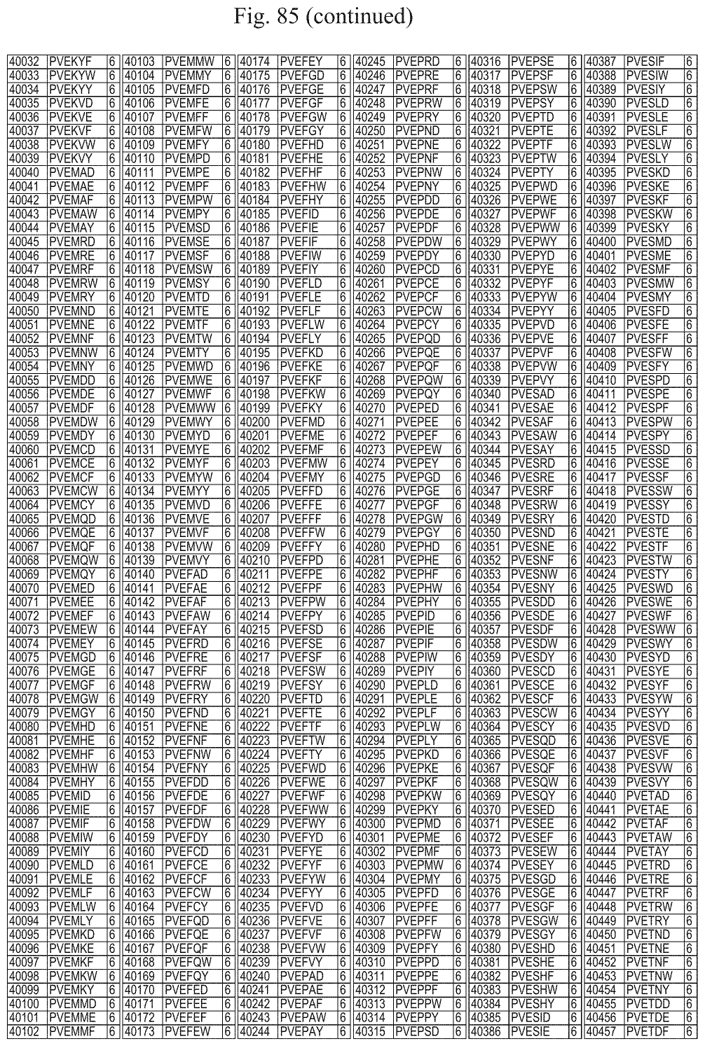

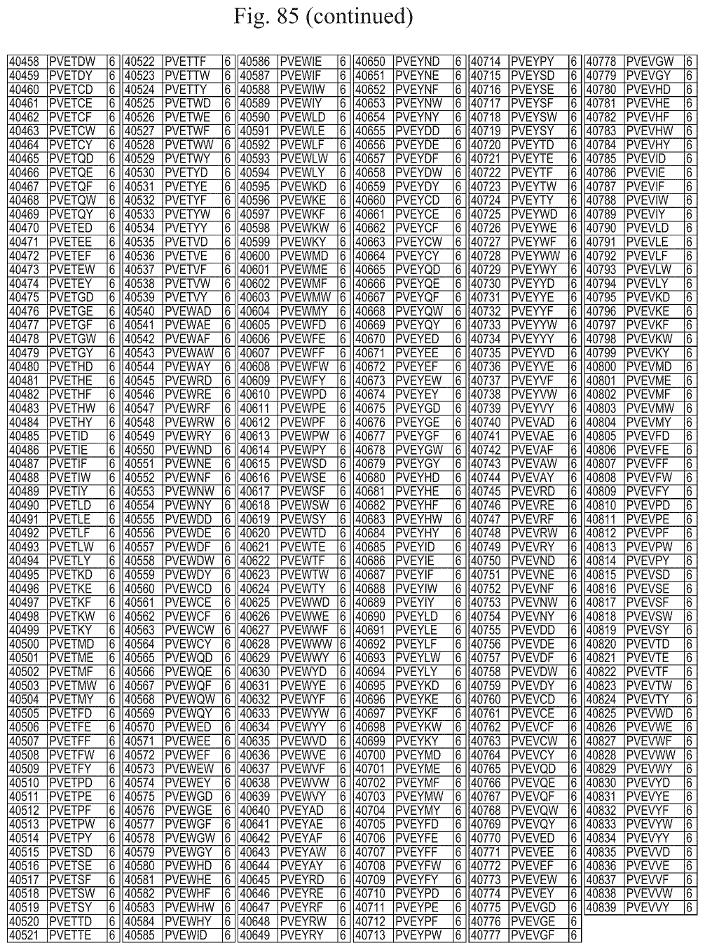

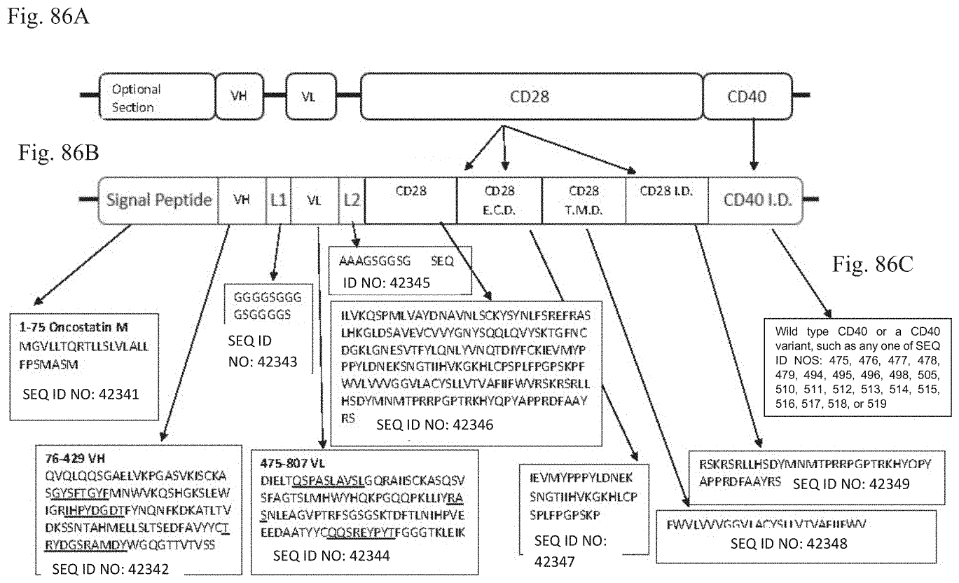

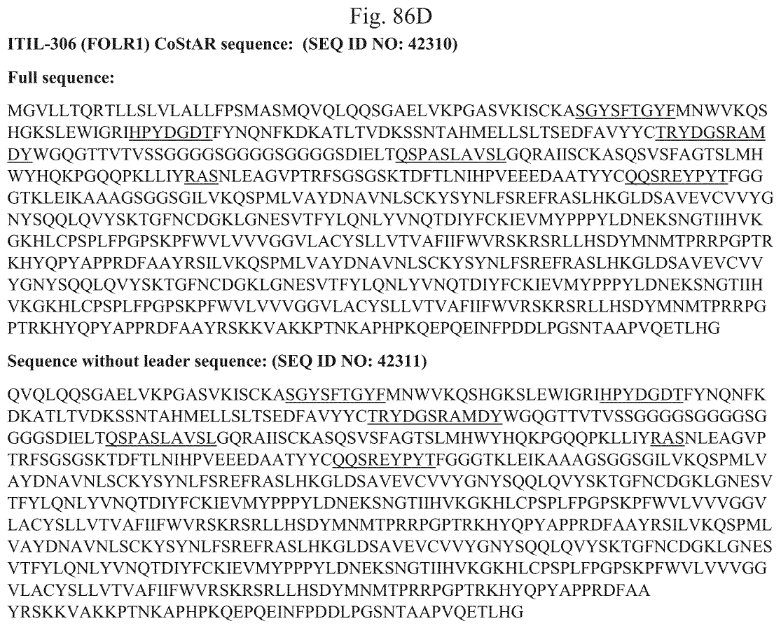

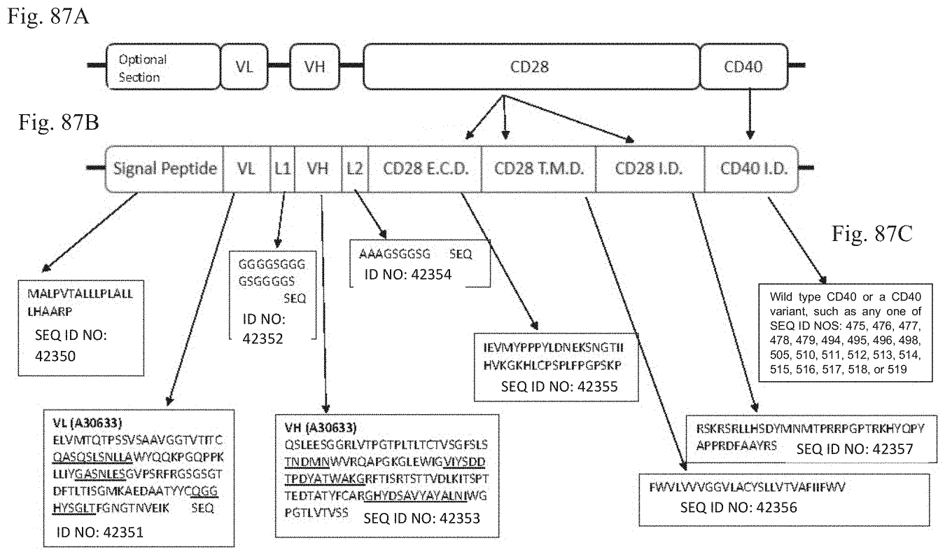

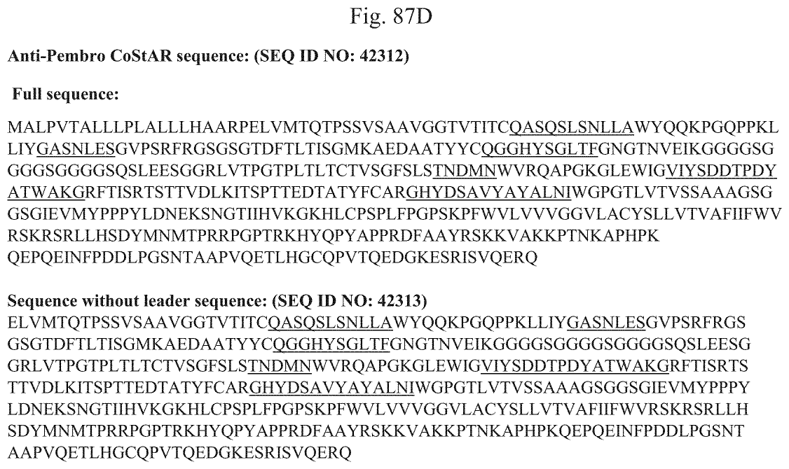

The disclosure herein relates to sequences that encode variants of TRAF binding domains. In some embodiments, these variants can be for TRAF2, TRAF2/3, or TRAF6 domains. In some embodiments, the TRAF2 variant will include one or more of the variants or point mutations provided herein. In some embodiments, the TRAF2/3 variant will include one or more of the variants or point mutations provided herein. In some embodiments, the TRAF6 variant will include one or more of the variants or point mutations provided herein. In some embodiments, the TRAF2, TRAF2/3, or TRAF6 variant will be any one or more of those provided herein, including, for example, one or more of SEQ ID Nos: 475, 476, 477, 478, 479, 494, 495, 496, 498, 505, 510, 511, 512, 513, 514, 515, 516, 517, 518, or 519. In some embodiments, the variant will comprise any one or more of the constructs from Table 2, Table 4, Table 5, or FIG. 67. In some embodiments, the variant is not one of the constructs of FIG. 78 (SEQ ID NOS: 40839-41074) or FIG. 79 (SEQ ID NOS: 41075-43474). In some embodiments, the variant comprises at least one of the constructs of FIG. 83, FIG. 84, or FIG. 85. In some embodiments, the TRAF2, TRAF2/3, or TRAF6 variant will be part of a CD40 protein. In some embodiments, the CD40 protein is part of a CoSTAR protein or other fusion construct as provided herein. In some embodiments, the TRAF variant is expressed in a T cell as provided herein. In some embodiments, the TRAF variant is expressed in a cell as shown in FIGS. 76A-76C. In some embodiments, the TRAF variant is part of a peptide construct as shown in FIGS. 77A-77C, 86A-86D, 87A-87D, 88A-88D, and/or 89A-89D.

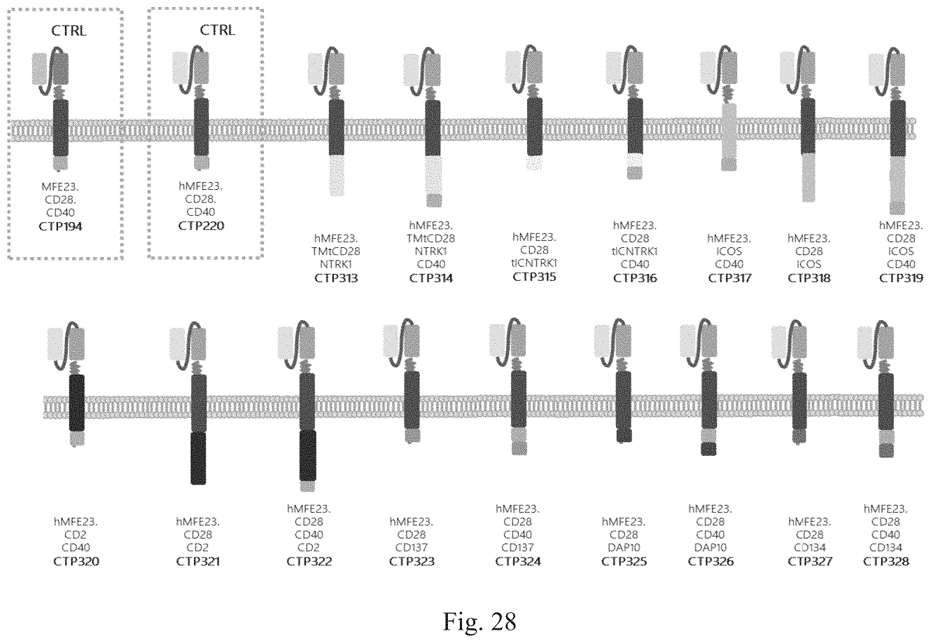

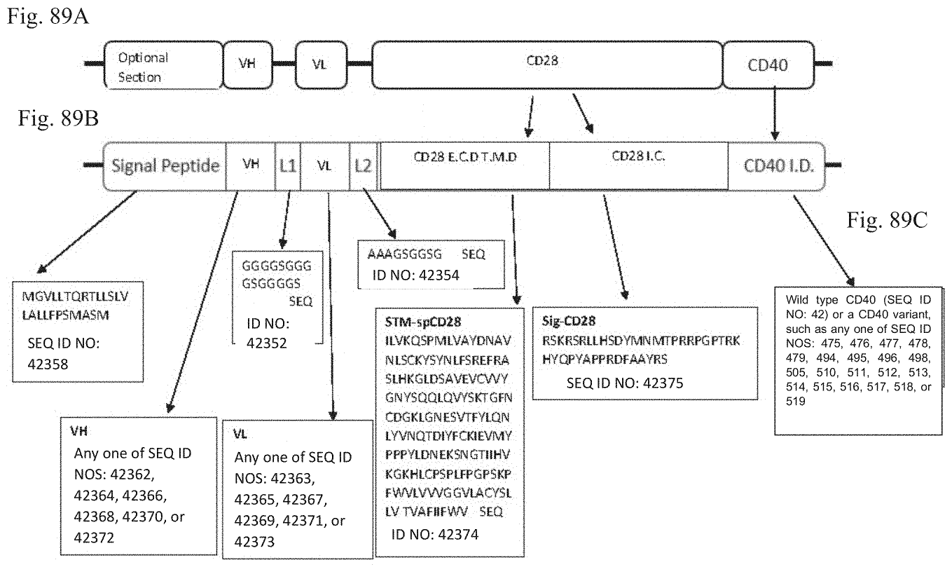

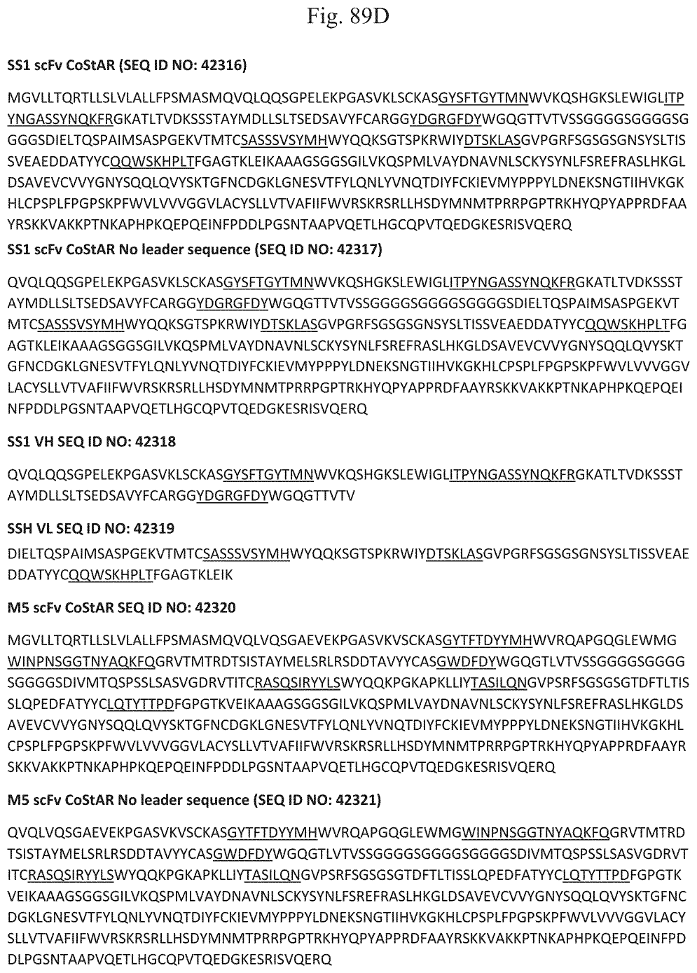

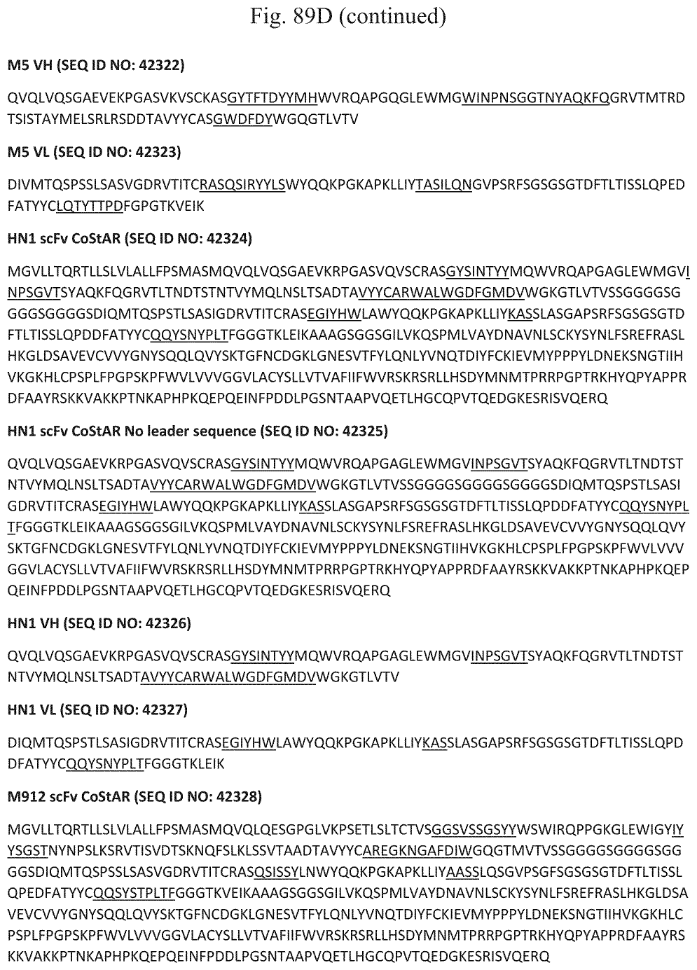

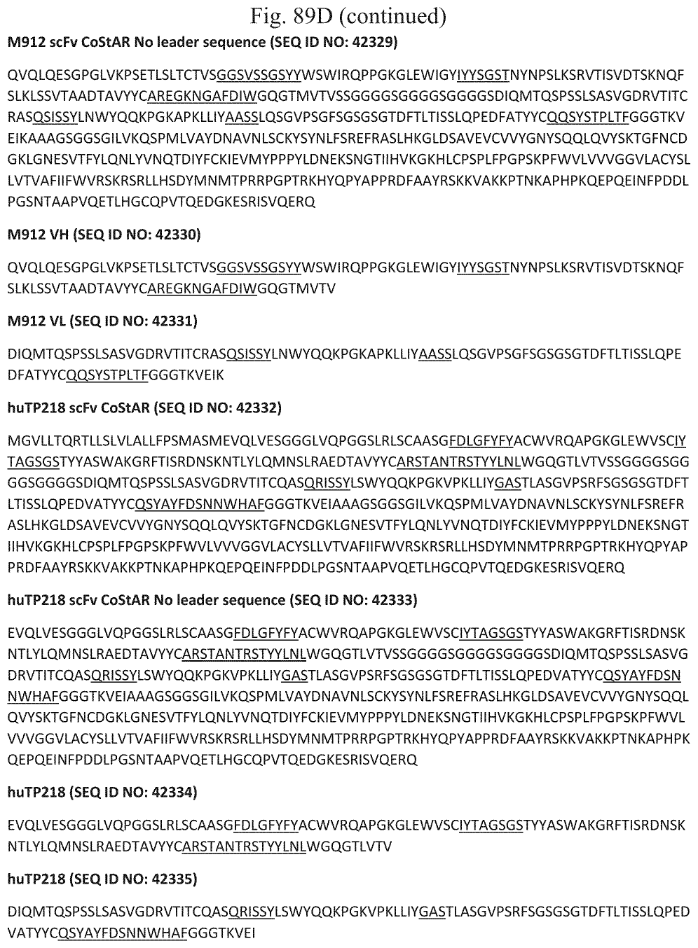

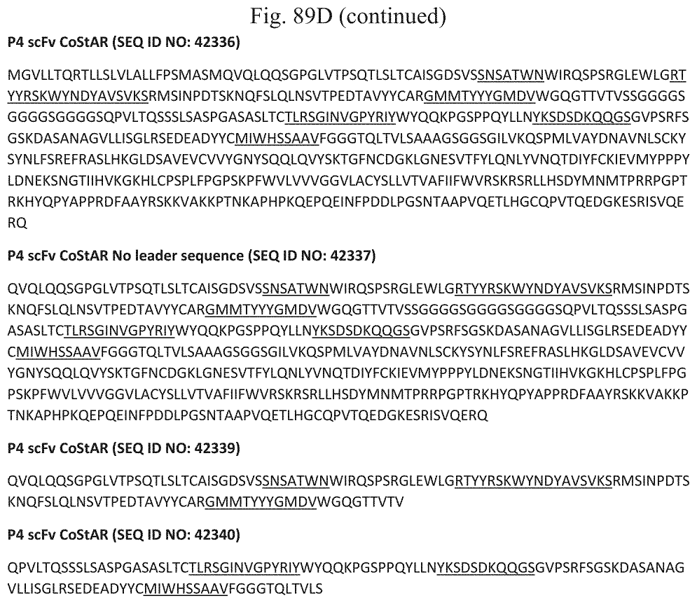

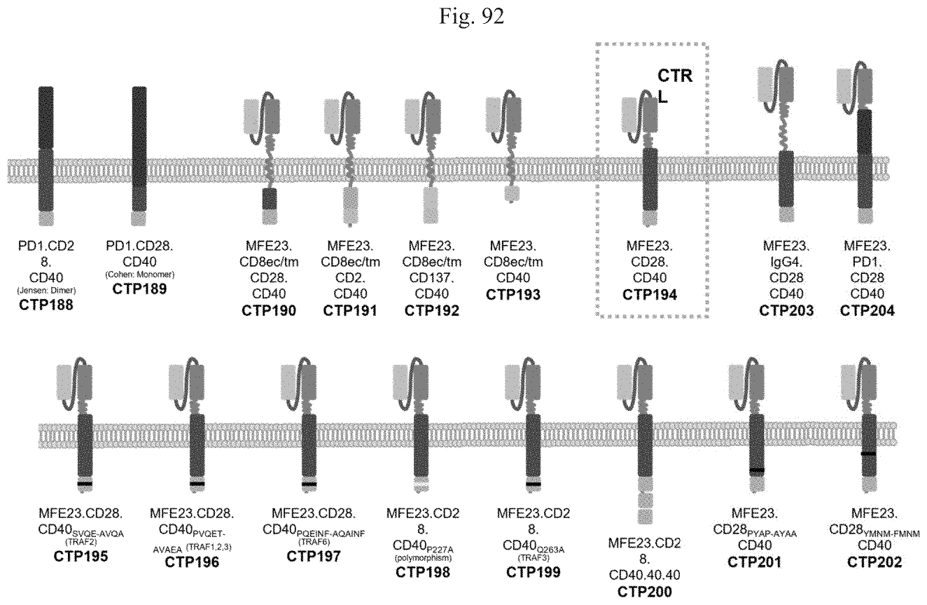

In some embodiments, any of the proteins and/or CoSTARs provided herein can employ a CD40 or TRAF variant instead of a wild-type CD40 or TRAF domain. Any of the CD40, TRAF2, TRAF2/3, or TRAF6 variants provided herein, including those in FIGS. 28, 67, 77A-77C, 83-89D, and 92 (excluding those in FIGS. 78 and 79), and including those in Tables 2, 4-5, and 7-14, and including SEQ ID NOS: 42, 475-479, 494-498, 505, 510-519, and 840-40839, and Consensus sequences A-C, can be used in the present method and/or construct and/or protein. For all descriptions of CD40, TRAF, TRAF2, TRAF2/3, and TRAF6 variants provided herein, in some embodiments, the sequences of FIGS. 78 and 79 are optionally to be excluded from the options of variants of the invention.

In some embodiments, the sequence is a variant of TRAF2. In some embodiments, the sequence is selected from any one of SEQ ID NOS: 520-679. In some embodiments, the sequence has about 60%, about 70%, about 80%, about 90%, about 95%, about 99%, about 100%, or any integer that is between about 60 and about 100%, identity to any one of SEQ ID NOS: 520-679. In some embodiments, the sequence is a variant of TRAF2/3. In some embodiments, the sequence is selected from any one of SEQ ID NOS: 680-839. In some embodiments, the sequence has about 60%, about 70%, about 80%, about 90%, about 95%, about 99%, about 100%, or any integer that is between about 60 and about 100%, identity to any one of SEQ ID NOS: 680-839. In some embodiments, the sequence is a variant of TRAF6. In some embodiments, the sequence is selected from any one of SEQ ID NOS: 840-40839. In some embodiments, the sequence has about 60%, about 70%, about 80%, about 90%, about 95%, about 99%, about 100%, or any integer that is between about 60 and about 100%, identity to any one of SEQ ID NOS: 840-40839. In some embodiments, the sequence comprises at least 2, 3, and/or 4 variants of TRAF.

In some embodiments, the sequence comprises a sequence from Table 2, 4, or 5. In some embodiments, the sequence comprises a sequence that has about 60%, about 70%, about 80%, about 90%, about 95%, about 99%, about 100%, or any integer that is between about 60 and about 100%, identity to a sequence from Table 2, 4, or 5. In some embodiments, the sequence is selected from any one of SEQ ID NOS: 475, 476, 477, 478, 479, 494, 495, 496, 498, 505, 510, 511, 512, 513, 514, 515, 516, 517, 518, and 519. In some embodiments, the sequence has about 60%, about 70%, about 80%, about 90%, about 95%, about 99%, about 100%, or any integer that is between about 60 and about 100%, identity to any one of SEQ ID NOS: 475, 476, 477, 478, 479, 494, 495, 496, 498, 505, 510, 511, 512, 513, 514, 515, 516, 517, 518, and 519.

Some embodiments of the present disclosure relate to a protein comprising an amino acid sequence comprising a sequence of Consensus A, or X₁X₂X₃X₄X₅X₆X₇X₈X₉X₁₀X₁₁. In some embodiments, X₁ is any amino acid, X₂ is any amino acid, X₃ is P, X₄ is any amino acid, X₅ is Q, X₆ is C, E, or A, X₇ is any amino acid, X₈ is any amino acid, X₉ is any amino acid, X₁₀ is any amino acid, and X₁₁ is any amino acid. In some embodiments, X₁, and X₂ are any amino acids, X₃ is P, X₄ is any amino acid, X₅ is Q, and X₆, X₇, X₈, X₉, X₁₀, and X₁₁ are any amino acids. In some embodiments, any one of X₁-X₁₁ is a naturally occurring amino acid. In some embodiments, any one of X₁-X₁₁ is a modified amino acid. In some embodiments, X₃ and/or X₅ is conservatively mutated to another amino acid with similar properties as P and/or Q, respectively. In some embodiments, the protein is part of a TRAF domain. In some embodiments, the protein is part of a TRAF2 domain. In some embodiments, the protein is part of a TRAF2/3 domain. In some embodiments, the sequence comprises: X₁ is S or P, X₂ is V, H, or Y, X₄ is I, Q, V, X₇ is T or S, X₈ is D, X₉ is K, D, or G, X₁₀ is T, S, G, or A, and X₁₁ is D, S, N, or D; or the sequence is at least 60%, at least 70%, at least 80%, at least 90%, at least 95%, at least 99%, and/or at least 100%, identical to (a). In some embodiments, the protein is of the sequence GSNTX₁X₂X₃X₄X₅X₆X₇X₈X₉X₁₀X₁₁, SEQ ID NO: 42299. In some embodiments, the protein is of the sequence X₁X₂X₃X₄X₅X₆X₇X₈X₉X₁₀X₁₁QPVT, SEQ ID NO: 42377. In some embodiments, the protein is of the sequence GSNTX₁X₂X₃X₄X₅X₆X₇X₈X₉X₁₀X₁₁QPVT, SEQ ID NO: 42378.

In some embodiments, provided is a protein comprising an amino acid sequence comprising a sequence of Consensus B, or X₁X₂X₃X₄X₅X₆X₇X₈X₉X₁₀X₁₁, wherein: X₁ is any amino acid, X₂ is any amino acid, X₃ is P, S, A, or T, X₄ is any amino acid, X₅ is Q or E, X₆ is E, X₇ is any amino acid, X₈ is any amino acid, X₉ is any amino acid, X₁₀ is any amino acid, and X₁₁ is any amino acid. In some embodiments, any one of X₁-X₁₁ is a naturally occurring amino acid. In some embodiments, any one of X₁-X₁₁ is a modified amino acid. In some embodiments, X₃ is conservatively mutated to another amino acid with similar properties as P, S, A, and/or T. In some embodiments, X₅ is conservatively mutated to another amino acid with similar properties as Q and/or E. In some embodiments, X₆ is conservatively mutated to another amino acid with similar properties as E. In some embodiments, the protein is part of a TRAF domain. In some embodiments, the protein is part of a TRAF2 domain. In some embodiments, the protein is part of a TRAF2/3 domain. In some embodiments, the sequence comprises: X₁ is P, A, M, T, S, R, V, H, X₂ is F, A, L, I, T, V, G, C, A, F, P, X₄ is K, V, I, H, A, Q, T, E, S, X₇ is C, T, E, D, S, A, X₈ is A, L, G, Y, I, Q, D, E, X₉ is F, H, K, R, P, A, G, Y, E, N, X₁₀ is R, G, E, K, A, S, D, C, C, P, V, and X₁₁ is S, C, D, P, W, T, A, G, E; or the sequence is at least 60%, at least 70%, at least 80%, at least 90%, at least 95%, at least 99%, and/or at least 100%, identical to (a). In some embodiments, the sequence is part of a Traf 2/3 sequence. In some embodiments, the protein is of the sequence GSNTX₁X₂X₃X₄X₅X₆X₇X₈X₉X₁₀X₁₁, SEQ ID NO: 42300. In some embodiments, the protein is of the sequence X₁X₂X₃X₄X₅X₆X₇X₈X₉X₁₀X₁₀QPVT, SEQ ID NO: 42379. In some embodiments, the protein is of the sequence GSNTX₁X₂X₃X₄X₅X₆X₇X₈X₉X₁₀X₁₁QPVT, SEQ ID NO: 42380.

In some embodiments, provided is a protein comprising an amino acid sequence comprising a sequence of Consensus C, or X₁X₂X₃X₄X₅X₆, wherein: X₁ is P, X₂ is any amino acid, X₃ is E, X₄ is any amino acid, X₅ is any amino acid, and X₆ is Ac/Ar. In some embodiments, X₁ is P, and X₂, X₃ X₄, X₅ and X₆ are any amino acids. In some embodiments, any one of X₁—X₆ is a naturally occurring amino acid. In some embodiments, any one of X₁—X₆ is a modified amino acid. In some embodiments, X₁ is conservatively mutated to another amino acid with similar properties as P. In some embodiments, X₆ is an aromatic amino acid. In some embodiments, X₆ is an acidic amino acid. In some embodiments, X₆ is conservatively mutated to another amino acid with similar properties as an acidic and/or aromatic amino acid. In some embodiments, X₆ is selected from Y, H, W, or F amino acids. In some embodiments, X₆ is selected from D or E amino acids. In some embodiments, the protein is part of a TRAF domain. In some embodiments, the protein is part of a TRAF6 domain. In some embodiments, the sequence comprises: X₂ is Q, P, E, V, T, or S, X₄ is I, L M, N, S, T, N, D, X₅ is N, D, R, S, G, or Y; or the sequence is at least 60%, at least 70%, at least 80%, at least 90%, at least 95%, at least 99%, and/or at least 100%, identical to (a). In some embodiments, the protein is of the sequence PKQEX₁X₂X₃X₄X₅X₆X₇X₈X₉X₁₀X₁₁, SEQ ID NO: 42301. In some embodiments, the protein is of the sequence X₁X₂X₃X₄X₅X₆PDDL, SEQ ID NO: 42381. In some embodiments, the protein is of the sequence PKQEX₁X₂X₃X₄X₅X₆PDDL, SEQ ID NO: 42382.

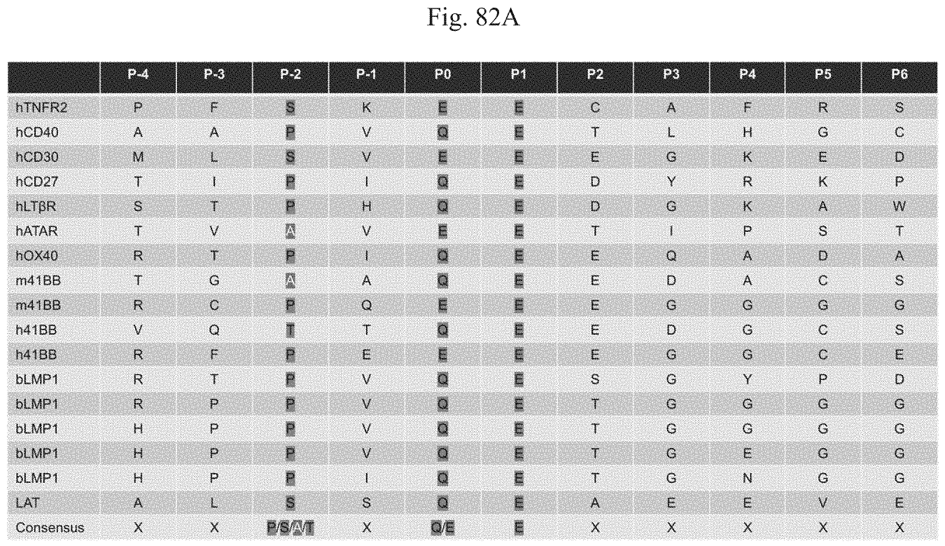

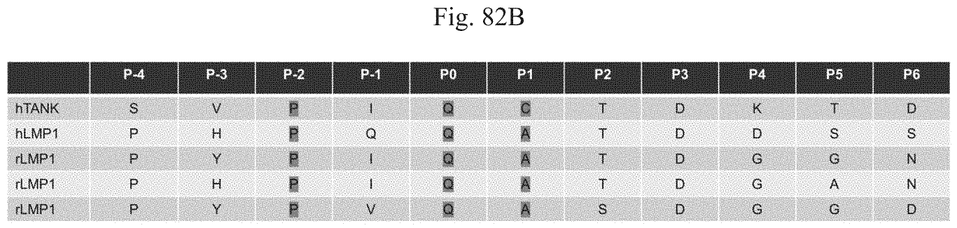

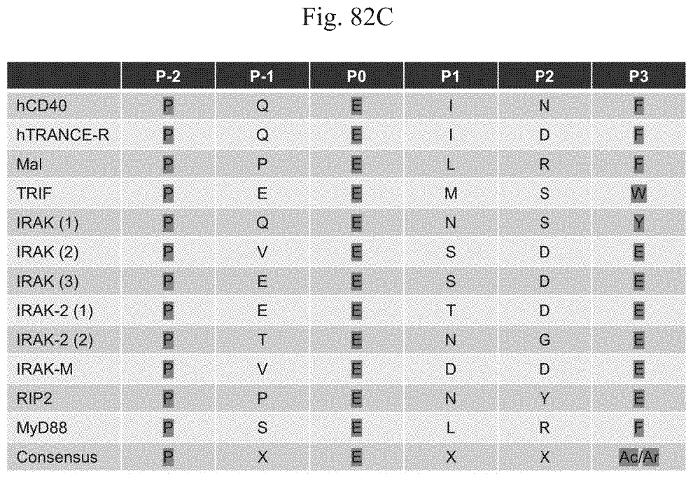

Consensus sequences A, B, and C were determined as shown in Tables 1 and 3 and FIGS. 82A-82C.

In some embodiments, the amino acid sequence comprises: (a) one or more of SEQ ID NOs: 475, 476, 477, 478, 479, 494, 495, 496, 498, 505, 510, 511, 512, 513, 514, 515, 516, 517, 518, or 519; or (b) a sequence that is at least 60%, at least 70%, at least 80%, at least 90%, at least 95%, at least 99%, and/or at least 100%, identical to (a).

In some embodiments the TRAF domain is in a CD40 protein, wherein the TRAF domain comprises one or more of: (a) SEQ ID NOs: 475, 476, 477, 478, 479, 494, 495, 496, 498, 505, 510, 511, 512, 513, 514, 515, 516, 517, 518, or 519; or (b) a sequence that is at least 60%, at least 70%, at least 80%, at least 90%, at least 95%, at least 99%, and/or at least 100%, identical to (a).

In some embodiments the TRAF6 domain sequence comprises: PQEINF, PEEMSW, PPENYE, or PQENSY. In some embodiments is a TRAF2/3 domain sequence comprising: PVQET, TQEET, SKEET, AVEET, or PVQET. In some embodiments is a TRAF2 domain sequence comprising: SVQE, AVEE or PEEE.

In some embodiments the TRAF 6 or TRAF2/3 domain comprises: (a) the amino acid sequence of one of SEQ ID NOs: 475, 476, 477, 478, 479, 494, 495, 496, 498, 505, 510, 511, 512, 513, 514, 515, 516, 517, 518, or 519; or (b) a sequence that is at least 60%, at least 70%, at least 80%, at least 90%, at least 95%, at least 99%, and/or at least 100%, identical to (a).

In some embodiments, the sequence is within a CD40 protein sequence for the protein, amino acid sequence, or TRAF domain of any one of the embodiments of the present disclosure. In some embodiments, the sequence is within a CD40 protein and is located at a corresponding TRAF 2, TRAF2/3, and/or TRAF6 domain within the CD40 protein.

In some embodiments, the TRAF2/TRAF3 domain comprises an amino acid sequence of Consensus A, or X₁X₂X₃X₄X₅X₆X₇X₈X₉X₁₀X₁₁, wherein X₁ is any amino acid, X₂ is any amino acid, X₃ is P, X₄ is any amino acid, X₅ is Q, X₆ is C, E, or A, X₇ is any amino acid, X₈ is any amino acid, X₉ is any amino acid, X₁₀ is any amino acid, and X₁₁ is any amino acid. In some embodiments, the protein is of the sequence GSNTX₁X₂X₃X₄X₅X₆X₇X₈X₉X₁₀X₁₁, SEQ ID NO: 42299. In some embodiments, the protein is of the sequence X₁X₂X₃X₄X₅X₆X₇X₈X₉X₁₀X₁₁QPVT, SEQ ID NO: 42377. In some embodiments, the protein is of the sequence GSNTX₁X₂X₃X₄X₅X₆X₇X₈X₉X₁₀X₁₁QPVT, SEQ ID NO: 42378.

In some embodiments the TRAF2/TRAF3 domain comprises an amino acid sequence of Consensus B, or. X₁X₂X₃X₄X₅X₆X₇X₈X₉X₁₀X₁₁, wherein X₁ is any amino acid, X₂ is any amino acid, X₃ is P, S, A, or T, X₄ is any amino acid, X₅ is Q or E, X₆ is E, X₇ is any amino acid, X₈ is any amino acid, X₉ is any amino acid, X₁₀ is any amino acid, and X₁₁ is any amino acid. In some embodiments, the protein is of the sequence GSNTX₁X₂X₃X₄X₅X₆X₇X₈X₉X₁₀X₁₁, SEQ ID NO: 42300. In some embodiments, the protein is of the sequence X₁X₂X₃X₄X₅X₆X₇X₈X₉X₁₀X₁₁QPVT, SEQ ID NO: 42379. In some embodiments, the protein is of the sequence GSNTX₁X₂X₃X₄X₅X₆X₇X₈X₉X₁₀X₁₁QPVT, SEQ ID NO: 42380.

In some embodiments the TRAF6 domain comprises an amino acid sequence of Consensus C, or X₁X₂X₃X₄X₅X₆, wherein X₁ is P, X₂ is any amino acid, X₃ is E, X₄ is any amino acid, X₅ is any amino acid, and X₆ is Ac/Ar. In some embodiments, the protein is of the sequence PKQEX₁X₂X₃X₄X₅X₆, SEQ ID NO: 42301. In some embodiments, the protein is of the sequence X₁X₂X₃X₄X₅X₆PDDL, SEQ ID NO: 42381. In some embodiments, the protein is of the sequence PKQEX₁X₂X₃X₄X₅X₆PDDL, SEQ ID NO: 42382.

In some embodiments the CD40 domain comprises an amino acid sequence of one or more of the following: (a) TRAF2/TRAF3 domain that comprises an amino acid sequence of Consensus A, or X₁X₂X₃X₄X₅X₆X₇X₈X₉X₁₀X₁₁, wherein X₁ is any amino acid, X₂ is any amino acid, X₃ is P, X₄ is any amino acid, X₅ is Q, X₆ is C, E, or A, X₇ is any amino acid, X₈ is any amino acid, X₉ is any amino acid, X₁₀ is any amino acid, and X₁₁ is any amino acid. (b) a TRAF2/TRAF3 domain that comprises an amino acid sequence of Consensus B, or X₁X₂X₃X₄X₅X₆X₇X₈X₉X₁₀X₁₁, wherein X₁ is any amino acid, X₂ is any amino acid, X₃ is P, S, A, or T, X₄ is any amino acid, X₅ is Q or E, X₆ is E, X₇ is any amino acid, X₈ is any amino acid, X₉ is any amino acid, X₁₀ is any amino acid, and X₁₁ is any amino acid. (c) a TRAF 6 domain that comprises an amino acid sequence of Consensus C, or X₁X₂X₃X₄X₅X₆, wherein X₁ is P, X₂ is any amino acid, X₃ is E, X₄ is any amino acid, X₅ is any amino acid, and X₆ is Ac/Ar.

In some embodiments, provided is a chimeric costimulatory antigen receptor (CoStAR) which comprises: an extracellular binding domain that binds to a tumor associated antigen; a transmembrane domain that is linked to the extracellular binding domain, a first signaling domain; and a second signaling domain, wherein the second signaling domain comprises a variant CD40 signaling domain or a signaling fragment thereof, wherein the variant CD40 comprises a sequence of a variant TRAF2/TRAF3 sequence, or a variant TRAF6 sequence, or a variant TRAF2 sequence, wherein the variant CD40 does not comprise SEQ ID NO: 42. In some embodiments, any of the CD40, TRAF2, TRAF2/3, or TRAF6 variants provided herein, including those in FIGS. 28, 67, 77A-77C, 83-89D, and 92 (excluding those in FIGS. 78 and 79), and including those in Tables 2, 4-5, and 7-14, and including SEQ ID NOS: 42, 475-479, 494-498, 505, 510-519, and 840-40839, and Consensus sequences A-C, can be used in the present method and/or construct and/or protein. The sequence options in FIGS. 78 and 79 can be optionally excluded from all of the embodiments provided herein.

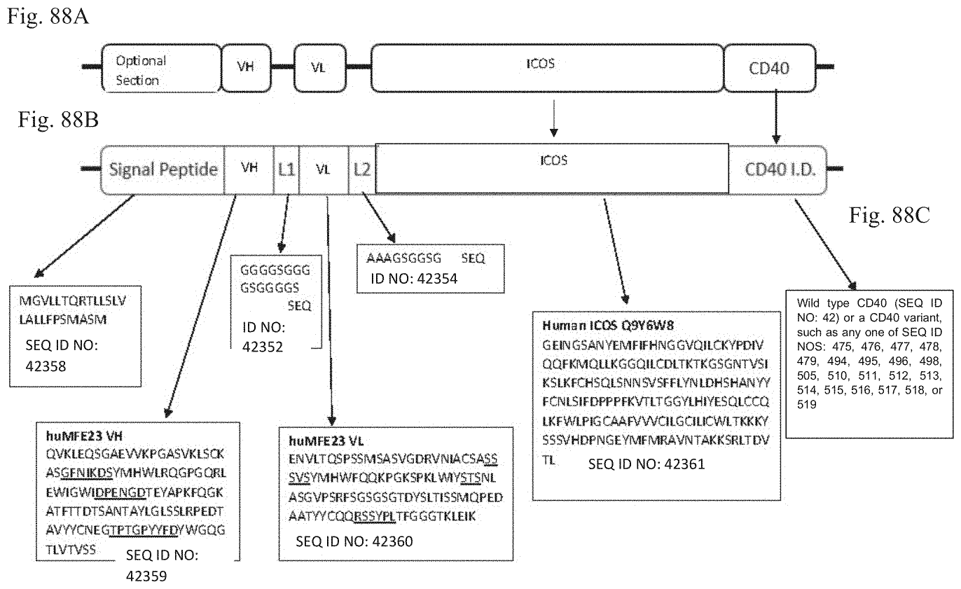

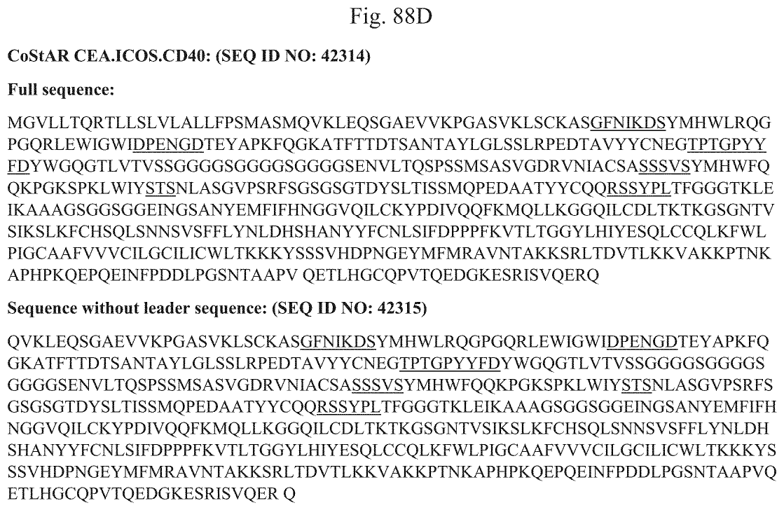

In some embodiments, provided is a chimeric costimulatory antigen receptor (CoStAR) which comprises: an extracellular binding domain that binds to carcinoembryonic antigen (CEA), or an extracellular binding domain that binds to mesothelin (MSLN); a transmembrane domain that is linked to the extracellular binding domain, a first signaling domain; and a second signaling domain, wherein the second signaling domain comprises a variant CD40 signaling domain or a signaling fragment thereof, wherein the variant CD40 comprises a sequence of a variant TRAF2/TRAF3 sequence, or a variant TRAF6 sequence, or a variant TRAF2 sequence, wherein the variant CD40 does not comprise SEQ ID NO: 42. In some embodiments, the first signaling domain comprises a signaling domain or signaling fragment of CD28 or CD278 (ICOS). In some embodiments, the first signaling domain comprises a full-length costimulatory domain. In some embodiments, the construct is as shown in FIG. 88A. In some embodiments, the construct is as shown in FIG. 88B. In some embodiments, the construct is as shown in FIG. 88C. In some embodiments, any of the CD40, TRAF2, TRAF2/3, or TRAF6 variants provided herein, including those in FIGS. 28, 67, 77A-77C, 83-89D, and 92 (excluding those in FIGS. 78 and 79), and including those in Tables 2, 4-5, and 7-14, and including SEQ ID NOS: 42, 475-479, 494-498, 505, 510-519, and 840-40839, and Consensus sequences A-C, can be used in the present method and/or construct and/or protein. The sequence options in FIGS. 78 and 79 can be optionally exclude from all of the embodiments provided herein.

In some embodiments, the extracellular binding domain is operatively linked to the transmembrane domain by a linker and/or a spacer. In some embodiments, the linker comprises about 2, 3, 5, 10, 15, 20, 25, 50, 75, 100, 150, 200, 250, 300, 350, or any integer that is between 2 and 350, amino acids. In some embodiments, the linker comprises from about 5 to about 20 amino acids. In some embodiments, the linker comprises from about 10 to about 250 amino acids. In some embodiments, the linker comprises 5 amino acids. In some embodiments, the linker comprises 10 amino acids. In some embodiments, the linker comprises 20 amino acids. In some embodiments, the linker comprises 50 amino acids. In some embodiments, the linker comprises 100 amino acids. In some embodiments, the linker comprises 250 amino acids.

In some embodiments, the transmembrane domain comprises a transmembrane domain from CD28, CD8, ICOS, DAP10, or NTRK. In some embodiments, the transmembrane domain comprises a transmembrane domain from CD28. In some embodiments, the transmembrane domain comprises a transmembrane domain from CD8. In some embodiments, the transmembrane domain comprises a transmembrane domain from ICOS. In some embodiments, the transmembrane domain comprises a transmembrane domain from DAP10. In some embodiments, the transmembrane domain comprises a transmembrane domain from NTRK. In some embodiments, the transmembrane domain comprises the transmembrane domain sequence of SEQ ID NO:20, SEQ ID NO:21, or SEQ ID NO:22. In some embodiments, the transmembrane domain comprises a sequence that has about 60%, about 70%, about 80%, about 90%, about 95%, about 99%, about 100%, or any integer that is between about 60 and about 100%, identity to any one SEQ ID NOS:20, SEQ ID NO:21, or SEQ ID NO:22. In some embodiments, the extracellular binding domain comprises an scFv, a peptide, an antibody heavy-chain variable domain, an antibody light-chain variable domain, or a CEA ligand. In some embodiments, any of the CD40, TRAF2, TRAF2/3, or TRAF6 variants provided herein, including those in FIGS. 28, 67, 77A-77C, 83-89D, and 92 (excluding those in FIGS. 78 and 79), and including those in Tables 2, 4-5, and 7-14, and including SEQ ID NOS: 42, 475-479, 494-498, 505, 510-519, and 840-40839, and Consensus sequences A-C, can be used in the present method and/or construct and/or protein. The sequence options in FIGS. 78 and 79 can be optionally excluded from all of the embodiments provided herein.

In some embodiments is a nucleic acid which encodes the CD40 TRAF variant construct of any one of the embodiments of the present disclosure.

In some embodiments is a nucleic acid which encodes the CoStAR of any one of the embodiments of the present disclosure.

In some embodiments is a vector comprising the nucleic acid of any one of the embodiments of the present disclosure.

In some embodiments is a cell which expresses the CD40 TRAF variant construct of any one of the embodiments of the present disclosure. In some embodiments, the cell comprises an alpha-beta T cell, gamma-delta T cell, T regulatory cell, TIL, NKT cell or NK cell. In some embodiments, the cell co-expresses a CAR or a TCR.

In some embodiments is a cell which expresses the CoStAR of any one of the embodiments of the present disclosure. In some embodiments, the cell comprises an alpha-beta T cell, gamma-delta T cell, T regulatory cell, TIL, NKT cell or NK cell. In some embodiments, the cell co-expresses a CAR or a TCR.

In some embodiments is a method of making the cell of any one of the embodiments of the present disclosure, which comprises the step of transducing or transfecting a cell with a vector of any one of the embodiments of the present disclosure

In some embodiments is a method for preparing a population of cells that express a CoStAR of any one the embodiments of the present disclosure, which comprises: detecting expression of the CoStAR on the surface of cells transfected or transduced with a vector of alternative 62; and selecting cells which are identified as expressing the CoStAR.

In some embodiments is a cell population which is enriched for cell expression a CoStAR of any one of the embodiments of the present disclosure.

In some embodiments is a method for treating a disease in a subject, which comprises the step of administering a cell according to any one of the embodiments of the present disclosure, or a cell population according to any one of the embodiments of the present disclosure, to the subject.

In some embodiments is a protein comprising the amino acid sequence of one of SEQ ID NOs: 510-519. In some embodiments, the protein comprises a sequence that has about 60%, about 70%, about 80%, about 90%, about 95%, about 99%, about 100%, or any integer that is between about 60 and about 100%, identity to any one SEQ ID NOS: SEQ ID NOs: 510-519. In some embodiments, the protein comprises the sequence of SEQ ID NO: 510. In some embodiments, the protein comprises a sequence that has about 60%, about 70%, about 80%, about 90%, about 95%, about 99%, about 100%, or any integer that is between about 60 and about 100%, identity to SEQ ID NO: 510. In some embodiments, the protein comprises the sequence of SEQ ID NO: 511. In some embodiments, the protein comprises a sequence that has about 60%, about 70%, about 80%, about 90%, about 95%, about 99%, about 100%, or any integer that is between about 60 and about 100%, identity to SEQ ID NO: 511. In some embodiments, the protein comprises the sequence of SEQ ID NO: 512. In some embodiments, the protein comprises a sequence that has about 60%, about 70%, about 80%, about 90%, about 95%, about 99%, about 100%, or any integer that is between about 60 and about 100%, identity to SEQ ID NO: 512. In some embodiments, the protein comprises the sequence of SEQ ID NO: 513. In some embodiments, the protein comprises a sequence that has about 60%, about 70%, about 80%, about 90%, about 95%, about 99%, about 100%, or any integer that is between about 60 and about 100%, identity to SEQ ID NO: 513. In some embodiments, the protein comprises the sequence of SEQ ID NO: 514. In some embodiments, the protein comprises a sequence that has about 60%, about 70%, about 80%, about 90%, about 95%, about 99%, about 100%, or any integer that is between about 60 and about 100%, identity to SEQ ID NO: 514. In some embodiments, the protein comprises the sequence of SEQ ID NO: 515. In some embodiments, the protein comprises a sequence that has about 60%, about 70%, about 80%, about 90%, about 95%, about 99%, about 100%, or any integer that is between about 60 and about 100%, identity to SEQ ID NO: 515. In some embodiments, the protein comprises the sequence of SEQ ID NO: 516. In some embodiments, the protein comprises a sequence that has about 60%, about 70%, about 80%, about 90%, about 95%, about 99%, about 100%, or any integer that is between about 60 and about 100%, identity to SEQ ID NO: 516. In some embodiments, the protein comprises the sequence of SEQ ID NO: 517. In some embodiments, the protein comprises a sequence that has about 60%, about 70%, about 80%, about 90%, about 95%, about 99%, about 100%, or any integer that is between about 60 and about 100%, identity to SEQ ID NO: 517. In some embodiments, the protein comprises the sequence of SEQ ID NO: 518. In some embodiments, the protein comprises a sequence that has about 60%, about 70%, about 80%, about 90%, about 95%, about 99%, about 100%, or any integer that is between about 60 and about 100%, identity to SEQ ID NO: 518. In some embodiments, the protein comprises the sequence of SEQ ID NO: 519. In some embodiments, the protein comprises a sequence that has about 60%, about 70%, about 80%, about 90%, about 95%, about 99%, about 100%, or any integer that is between about 60 and about 100%, identity to SEQ ID NO: 519. In some embodiments, any of the CD40, TRAF2, TRAF2/3, or TRAF6 variants provided herein, including those in FIGS. 28, 67, 77A-77C, 83-89D, and 92 (excluding those in FIGS. 78 and 79), and including those in Tables 2, 4-5, and 7-14, and including SEQ ID NOS: 42, 475-479, 494-498, 505, 510-519, and 840-40839, and Consensus sequences A-C, can be used in the present method and/or construct and/or protein. The sequence options in FIGS. 78 and 79 can be optionally excluded from all of the embodiments provided herein.

In some embodiments is an immune cell comprising an extracellular tumor-associated antigen (TAA) specific binding domain and a transmembrane domain, comprising: a) a CD28 intracellular signalling domain; and b) a CD40 intracellular signalling domain, wherein the CD40 intracellular signalling domain comprises a TRAF variant of any one of the embodiments of the present disclosure. In some embodiments is an isolated nucleic acid molecule encoding the chimeric receptor of an immune cell comprising an extracellular tumor-associated antigen (TAA) specific binding domain and a transmembrane domain, wherein the nucleic acid molecule further comprises a TRAF variant of any one of the embodiments of the present disclosure. In some embodiments, any of the CD40, TRAF2, TRAF2/3, or TRAF6 variants provided herein, including those in FIGS. 28, 67, 77A-77C, 83-89D, and 92 (excluding those in FIGS. 78 and 79), and including those in Tables 2, 4-5, and 7-14, and including SEQ ID NOS: 42, 475-479, 494-498, 505, 510-519, and 840-40839, and Consensus sequences A-C, can be used in the present method and/or construct and/or protein. The sequence options in FIGS. 78 and 79 can be optionally excluded from all of the embodiments provided herein.

In some embodiments is a chimeric costimulatory receptor (CoStAR) comprising an extracellular ligand binding fragment of a first costimulatory receptor and an intracellular signalling fragment of a second costimulatory receptor fused to a tumour associated antigen specific binding domain linked to a signalling domain, wherein the CoStAR further comprises a TRAF variant of any one of the embodiments of the present disclosure. In some embodiments is a CoStAR comprising an antigen specific binding domain, which is selected from: (a) a single chain antibody fragment which binds a tumour, associated antigen including, but not limited to, carcinoembryonic antigen (CEA), 5T4, melanotransferrin (CD228), Her2, EGFR, GPC3, melanoma-associated chondroitin sulphate proteoglycan (MCSP/CSPG4), CD71, folate receptor or CA125; or (b) a single chain antibody fragment which binds a tumour specific peptide (p)-major histocompatibility (MHO) complex; or (c) a tumour specific pMHC complex antigen specific single chain T-cell receptor (scTCR); or (d) a natural antigen binding polypeptide such as, but not limited to, transferrin; or (e) a domain which bind an antibody (e.g. Fe binding domains such as, but not limited to, CD16, CD32 or CD64), or other indirect method of antigen recognition. In some embodiments, the CoStAr is fused to at least one of: (1) a fusion signalling domain comprising or consisting of full length human CD28, or a variant thereof having at least 60%, 70%, 80%, 90%, 95%, 99%, 100%, or any integer that is between 60 and 100%, sequence identity at the protein level; (2) the intracellular domain from human CD137, such as or a variant thereof having at least 60%, 70%, 80%, 90%, 95%, 99%, 100%, or any integer that is between 60 and 100%, sequence identity at the protein level, (3) the intracellular domain from human CD134, or a variant thereof having at least 60%, 70%, 80%, 90%, 95%, 99%, 100%, or any integer that is between 60 and 100%, sequence identity at the protein level, (4) the intracellular domain from human CD2, or a variant thereof having at least 60%, 70%, 80%, 90%, 95%, 99%, 100%, or any integer that is between 60 and 100%, sequence identity at the protein level, (5) the intracellular domain from human CD29, or a variant thereof having at least 60%, 70%, 80%, 90%, 95%, 99%, 100%, or any integer that is between 60 and 100%, sequence identity at the protein level, (6) the intracellular domain from human GITR, or a variant thereof having at least 60%, 70%, 80%, 90%, 95%, 99%, 100%, or any integer that is between 60 and 100%, sequence identity at the protein level, (7) the intracellular domain from human IL2R-gamma, or a variant thereof having at least 60%, 70%, 80%, 90%, 95%, 99%, 100%, or any integer that is between 60 and 100%, sequence identity at the protein level, (8) the intracellular domain from human CD40, or a variant thereof having at least 60%, 70%, 80%, 90%, 95%, 99%, 100%, or any integer that is between 60 and 100%, sequence identity at the protein level, (9) the intracellular domain from human CD150, or a variant thereof having at least 60%, 70%, 80%, 90%, 95%, 99%, 100%, or any integer that is between 60 and 100%, sequence identity at the protein level, and/or (10) the intracellular domain from human CD2 and human CD40, or a variant thereof having at least 60%, 70%, 80%, 90%, 95%, 99%, 100%, or any integer that is between 60 and 100%, sequence identity at the protein level. In some embodiments, any of the CD40, TRAF2, TRAF2/3, or TRAF6 variants provided herein, including those in FIGS. 28, 67, 77A-77C, 83-89D, and 92 (excluding those in FIGS. 78 and 79), and including those in Tables 2, 4-5, and 7-14, and including SEQ ID NOS: 42, 475-479, 494-498, 505, 510-519, and 840-40839, and Consensus sequences A-C, can be used in the present method and/or construct and/or protein. The sequence options in FIGS. 78 and 79 can be optionally excluded from all of the embodiments provided herein.

In some embodiments is a chimeric costimulatory antigen receptor (CoStAR) which comprises: an extracellular binding domain operatively linked to a transmembrane domain, a CD28 signaling domain and a CD40 signaling domain, wherein the at least one extracellular binding domain comprises the amino acid sequence of SEQ ID NO:42275, wherein the CD28 signaling domain comprises the amino acid sequence of SEQ ID NO: 42276, and wherein the CD40 signaling domain comprises the amino acid sequence of SEQ ID NO: 42277. In some embodiments the CoStAR further comprises at least one TRAF variant of any one of the embodiments of the present disclosure. In some embodiments is a protein that comprises: a first portion that comprises the amino acid sequence of SEQ ID NO:42275 that is linked to; a second portion that comprises a transmembrane domain that is linked to; a third portion that comprises the amino acid sequence of SEQ ID NO: 42276 that is linked to; a fourth portion that comprises the amino acid sequence of SEQ ID NO: 42277. In some embodiments the protein further comprises at least one TRAF variant of any one of the embodiments of the present disclosure. In some embodiments is a protein that comprises: a first portion that comprises the amino acid sequence of SEQ ID NO: 42275; a second portion that comprises the amino acid sequence of SEQ ID NO: 42278; a third portion that comprises the amino acid sequence of SEQ ID NO: 42276; a fourth portion that comprises the amino acid sequence of SEQ ID NO: 42277; and a fifth portion that comprises SEQ ID NO: 42279, wherein the first portion is linked to the second portion by the fifth portion, wherein the third portion is linked to the second portion, and wherein the fourth portion is linked to the third portion. In some embodiments the protein further comprises at least one TRAF variant of any one of the embodiments of the present disclosure. In some embodiments, any of the CD40, TRAF2, TRAF2/3, or TRAF6 variants provided herein, including those in FIGS. 28, 67, 77A-77C, 83-89D, and 92 (excluding those in FIGS. 78 and 79), and including those in Tables 2, 4-5, and 7-14, and including SEQ ID NOS: 42, 475-479, 494-498, 505, 510-519, and 840-40839, and Consensus sequences A-C, can be used in the present method and/or construct and/or protein. The sequence options in FIGS. 78 and 79 can be optionally excluded from all of the embodiments provided herein.

In some embodiments is a chimeric costimulatory antigen receptor (CoStAR) which comprises: an extracellular binding domain comprising a VH of antibody 196-14 and a VL of antibody 196-14, operatively linked to a transmembrane domain, a CD28 signaling domain and a CD40 signaling domain, wherein the CD28 signaling domain comprises the amino acid sequence of SEQ ID NO: 42276, and wherein the CD40 signaling domain comprises the amino acid sequence of SEQ ID NO: 42277. In some embodiments the CoStAR further comprises at least one TRAF variant of any one of the embodiments of the present disclosure. In some embodiments is a cell population enriched for cell expression of a CoStAR, the CoStAR comprising: an extracellular binding domain comprising a VH of antibody 196-14 and a VL of antibody 196-14, operatively linked to a transmembrane domain, a CD28 signaling domain and a CD40 signaling domain, wherein the CD28 signaling domain comprises the amino acid sequence of SEQ ID NO: 42276, and wherein the CD40 signaling domain comprises the amino acid sequence of SEQ ID NO: 42277. In some embodiments the CoStAR further comprises at least one TRAF variant of any one of the embodiments of the present disclosure. In some embodiments, any of the CD40, TRAF2, TRAF2/3, or TRAF6 variants provided herein, including those in FIGS. 28, 67, 77A-77C, 83-89D, and 92 (excluding those in FIGS. 78 and 79), and including those in Tables 2, 4-5, and 7-14, and including SEQ ID NOS: 42, 475-479, 494-498, 505, 510-519, and 840-40839, and Consensus sequences A-C, can be used in the present method and/or construct and/or protein. The sequence options in FIGS. 78 and 79 can be optionally excluded from all of the embodiments provided herein.

In some embodiments is a CoStAR which comprises: an extracellular binding domain operatively linked to a transmembrane domain, a first signaling domain, a CD40 signaling domain or a fragment thereof, and at least one TRAF variant of any one of the embodiments of the present disclosure. In some embodiments, the CD40 signaling fragment comprises an SH3 motif (KPTNKAPH, SEQ ID NO: 42280), TRAF2 motif (PKQE, SEQ ID NO: 42281, PVQE, SEQ ID NO: 42282, SVQE, SEQ ID NO: 42283), TRAF6 motif (QEPQEINFP, SEQ ID NO: 42284), PKA motif (KKPTNKA, SEQ ID NO: 42285, SRISVQE, SEQ ID NO: 42286), or a combination thereof, or is a full length CD40 intracellular domain. In some embodiments, the first signaling domain comprises a signaling domain or signaling fragment of CD2, CD9, CD26, CD27, CD28, CD29, CD38, CD40, CD43, CD46, CD49d, CD55, CD73, CD81, CD82, CD99, CD100, CD134 (OX40), CD137 (41BB), CD150 (SLAM), CD270 (HVEM), CD278 (ICOS), CD357 (GITR), or EphB6. In some embodiments, the extracellular binding domain binds to CD70, CD146, FOLRI, carcinoembryonic antigen (CEA), 5T4, mellanotransferrin (CD228), Her2, EGFR, GPC3, melanoma-associated chondroitin sulphate proteoglycan (MCSP/CSPG4), CD71, EPCAM, SM5-1, folate receptor or CA125, PDL-1, CD155 PD-I, mesothelin, or a tumor specific peptide (p)-major histocompatability (MHC) complex, or a tumor specific pMHC complex antigen specific single chain T-cell receptor (scTCR), or transferrin, or an antibody or antigen binding protein. In some embodiments, any of the CD40, TRAF2, TRAF2/3, or TRAF6 variants provided herein, including those in FIGS. 28, 67, 77A-77C, 83-89D, and 92 (excluding those in FIGS. 78 and 79), and including those in Tables 2, 4-5, and 7-14, and including SEQ ID NOS: 42, 475-479, 494-498, 505, 510-519, and 840-40839, and Consensus sequences A-C, can be used in the present method and/or construct and/or protein. The sequence options in FIGS. 78 and 79 can be optionally excluded from all of the embodiments provided herein.

In some embodiments is a chimeric costimulatory antigen receptor (CoStAR) which comprises: an extracellular binding domain specific for a tumor associated antigen (TAA) operatively linked to a transmembrane domain, a CD28 signaling domain, a CD40 signaling domain or a signaling fragment thereof, and at least one TRAF variant of any one of the embodiments of the present disclosure. In some embodiments, the CoStAR is specific for FOLR1. In some embodiments, the CoStAR is specific for PDL1. In some embodiments, the CoStAR is specific for CEA. In some embodiments, the extracellular binding domain comprises MOV19 scFv. In some embodiments, the extracellular binding domain is specific for CD70, CD146, FOLR1, carcinoembryonic antigen (CEA), 5T4, mellanotransferrin (CD228), Her2, EGFR, GPC3, melanoma-associated chondroitin sulphate proteoglycan (MCSP/CSPG4), CD71, EPCAM, SM5-1, folate receptor or CA125, PDL-1, CD155 PD-1, mesothelin, or a tumor specific peptide (p)-major histocompatability (MHC) complex, or a tumor specific pMHC complex antigen specific single chain T-cell receptor (scTCR), or transferrin, or an antibody or antigen binding protein. In some embodiments, any of the CD40, TRAF2, TRAF2/3, or TRAF6 variants provided herein, including those in FIGS. 28, 67, 77A-77C, 83-89D, and 92 (excluding those in FIGS. 78 and 79), and including those in Tables 2, 4-5, and 7-14, and including SEQ ID NOS: 42, 475-479, 494-498, 505, 510-519, and 840-40839, and Consensus sequences A-C, can be used in the present method and/or construct and/or protein. The sequence options in FIGS. 78 and 79 can be optionally excluded from all of the embodiments provided herein.

In some embodiments is a fusion protein comprising: at least one TRAF variant of any one of the embodiments of the present disclosure, (a) a binding domain, wherein the binding domain comprises: an L-CDR1 sequence comprising the amino acid sequence of SEQ ID NO: 42287, an L-CDR2 sequence comprising the amino acid sequence of SEQ ID NO: 42288, an L-CDR3 sequence comprising the amino acid sequence of SEQ ID NO: 42289, an H-CDR1 sequence comprising the amino acid sequence of SEQ ID NO: 42290, an H-CDR2 sequence comprising the amino acid sequence of SEQ ID NO: 42291, and an H-CDR3 sequence comprising the amino acid sequence of SEQ ID NO: 173; (b) an amino acid sequence of SEQ ID NO: 42293 linked to the binding domain; (c) an amino acid sequence of SEQ ID NO: 42294 linked to (b); (d) an amino acid sequence of SEQ ID NO: 42295 linked to (c); and (e) an amino acid sequence of SEQ ID NO: 42296 linked to (d). In some embodiments is a fusion protein comprising the amino acid sequence of SEQ ID NO: 42297 or 42298, and at least one TRAF variant of any one of the embodiments of the present disclosure. In some embodiments, any of the CD40, TRAF2, TRAF2/3, or TRAF6 variants provided herein, including those in FIGS. 28, 67, 77A-77C, 83-89D, and 92 (excluding those in FIGS. 78 and 79), and including those in Tables 2, 4-5, and 7-14, and including SEQ ID NOS: 42, 475-479, 494-498, 505, 510-519, and 840-40839, and Consensus sequences A-C, can be used in the present method and/or construct and/or protein. The sequence options in FIGS. 78 and 79 can be optionally excluded from all of the embodiments provided herein.

In some embodiments is an engineered protein comprising a clustering domain and a signaling domain comprising a CD40 signaling domain or signaling fragment thereof and at least one TRAF variant of any one of the embodiments of the present disclosure is provided, wherein the clustering domain is capable of oligomerization whereby the signaling domain is activated. In some embodiments the protein comprises a constitutively stimulating antigen agnostic receptor (C-SAAR). In some embodiments, the protein comprises at least one of: LZ(cFos)-EGFRTM/JMD-CD28-CD40, LZ(cFos)-CD28TM-CD28-CD40, LZ(cJun)-EGFRTM/JMDCD28-CD40, LZ(cJun)-CD28TM-CD28-CD40, LZ(c/EBP)-EGFRTM/JMD-CD28-CD40, or LZ(c/EBP)-CD28TM-CD28-CD40. In some embodiments, the protein further comprises an inducible costimulatory receptor. In some embodiments, the protein comprises at least one of: anti-ID1-VHVL(A3 0514-pembro)-CD28TMD-CD28-CD40, or Anti-ID 1-VL-VH(A3 0514-pembro)CD28TMD-CD28-CD40, or Anti-ID2-VH-VL(A3 0523-pembro)-CD28TMD-CD28-CD40, or anti-ID2-VL-Vh(A3 0523-pembro)-CD28TMD-CD28-CD40, or Anti-ID3-Vh-VL(A3063 3-pembro)-CD28TMD-CD28-CD40, or Anti-ID3-VL-VH(A 3063 3-pembro)-CD28TMD-CD28-CD40. In some embodiments, the engineered protein comprises a transmembrane domain and the clustering domain oligomerizes when the engineered protein is bound by a ligand. In some embodiments, any of the CD40, TRAF2, TRAF2/3, or TRAF6 variants provided herein, including those in FIGS. 28, 67, 77A-77C, 83-89D, and 92 (excluding those in FIGS. 78 and 79), and including those in Tables 2, 4-5, and 7-14, and including SEQ ID NOS: 42, 475-479, 494-498, 505, 510-519, and 840-40839, and Consensus sequences A-C, can be used in the present method and/or construct and/or protein. The sequence options in FIGS. 78 and 79 can be optionally excluded from all of the embodiments provided herein.

In some embodiments is a CoStAR which comprises an extracellular binding domain that binds to carcinoembryonic antigen (CEA) and at least one TRAF variant of any one of the embodiments of the present disclosure. In some embodiments, the extracellular binding domain is operatively linked to ICOS, wherein ICOS comprises the amino acid sequence of SEQ ID NO: 515, wherein ICOS is linked to a CD40 signaling domain, and wherein the extracellular binding domain comprises: a HCDR1 that is an HCDR1 in SEQ ID NO: 12; a HCDR2 that is an HCDR2 in SEQ ID NO: 12; a HCDR3 that is an HCDR3 in SEQ ID NO: 12; a LCDR1 that is an LCDR1 in SEQ ID NO: 12; a LCDR2 that is an LCDR2 in SEQ ID NO: 12; and a LCDR3 that is an HCDR3 in SEQ ID NO: 12. In some embodiments is a fusion protein comprising at least one TRAF variant of any one of the embodiments of the present disclosure, wherein the fusion protein further comprises a HCDR1 that is an HCDR1 in SEQ ID NO: 12; a HCDR2 that is an HCDR2 in SEQ ID NO: 12; a HCDR3 that is an HCDR3 in SEQ ID NO: 12; a LCDR1 that is an LCDR1 in SEQ ID NO: 12; a LCDR2 that is an LCDR2 in SEQ ID NO: 12; and a LCDR3 that is an HCDR3 in SEQ ID NO: 12. In some embodiments, the construct is as shown in FIG. 88A. In some embodiments, the construct is as shown in FIG. 88B. In some embodiments, the construct is as shown in FIG. 88C. In some embodiments, any of the CD40, TRAF2, TRAF2/3, or TRAF6 variants provided herein, including those in FIGS. 28, 67, 77A-77C, 83-89D, and 92 (excluding those in FIGS. 78 and 79), and including those in Tables 2, 4-5, and 7-14, and including SEQ ID NOS: 42, 475-479, 494-498, 505, 510-519, and 840-40839, and Consensus sequences A-C, can be used in the present method and/or construct and/or protein. The sequence options in FIGS. 78 and 79 can be optionally excluded from all of the embodiments provided herein.

In some embodiments is a CoStAR which comprises: an extracellular binding domain that binds to mesothelin (MSLN) and at least one TRAF variant of any one of the embodiments of the present disclosure. In some embodiments, the CoStAR further comprises a transmembrane domain; a CD28 signaling domain, wherein the CD28 signaling domain comprises the amino acid sequence of SEQ ID NO: 25; and a CD40 signaling domain, wherein the extracellular binding domain comprises any one of: a HCDR1 that is an HCDR1 in SEQ ID NO: 186, a HCDR2 that is an HCDR2 in SEQ ID NO: 186, a HCDR3 that is an HCDR3 in SEQ ID NO: 186, a LCDR1 that is an LCDR1 in SEQ ID NO: 186, a LCDR2 that is an LCDR2 in SEQ ID NO: 186, and LCDR3 that is an LCDR3 in SEQ ID NO: 186; a HCDR1 that is an HCDR1 in SEQ ID NO: 187, a HCDR2 that is an HCDR2 in SEQ ID NO: 187, a HCDR3 that is an HCDR3 in SEQ ID NO: 187, a LCDR1 that is an LCDR1 in SEQ ID NO: 187, a LCDR2 that is an LCDR2 in SEQ ID NO: 187, and LCDR3 that is an LCDR3 in SEQ ID NO: 187; a HCDR1 that is an HCDR1 in SEQ ID NO: 188, a HCDR2 that is an HCDR2 in SEQ ID NO: 188, a HCDR3 that is an HCDR3 in SEQ ID NO: 188, a LCDR1 that is an LCDR1 in SEQ ID NO: 188, a LCDR2 that is an LCDR2 in SEQ ID NO: 188, and LCDR3 that is an LCDR3 in SEQ ID NO: 188; a HCDR1 that is an HCDR1 in SEQ ID NO: 189, a HCDR2 that is an HCDR2 in SEQ ID NO: 189, a HCDR3 that is an HCDR3 in SEQ ID NO: 189, a LCDR1 that is an LCDR1 in SEQ ID NO: 189, a LCDR2 that is an LCDR2 in SEQ ID NO: 189, and LCDR3 that is an LCDR3 in SEQ ID NO: 189; a HCDR1 that is an HCDR1 in SEQ ID NO: 190, a HCDR2 that is an HCDR2 in SEQ ID NO: 190, a HCDR3 that is an HCDR3 in SEQ ID NO: 190, a LCDR1 that is an LCDR1 in SEQ ID NO: 190, a LCDR2 that is an LCDR2 in SEQ ID NO: 190, and LCDR3 that is an LCDR3 in SEQ ID NO: 190; a HCDR1 that is an HCDR1 in SEQ ID NO: 191, a HCDR2 that is an HCDR2 in SEQ ID NO: 191, a HCDR3 that is an HCDR3 in SEQ ID NO: 191, a LCDR1 that is an LCDR1 in SEQ ID NO: 191, a LCDR2 that is an LCDR2 in SEQ ID NO: 191, and LCDR3 that is an LCDR3 in SEQ ID NO: 191; a VH that is a VH in SEQ ID NO: 186, and a VL that is a VL in SEQ ID NO: 186; a VH that is a VH in SEQ ID NO: 187, and a VL that is a VL in SEQ ID NO: 187; a VH that is a VH in SEQ ID NO: 188, and a VL that is a VL in SEQ ID NO: 188; a VH that is a VH in SEQ ID NO: 189, and a VL that is a VL in SEQ ID NO: 189; a VH that is a VH in SEQ ID NO: 190, and a VL that is a VL in SEQ ID NO: 190; or a VH that is a VH in SEQ ID NO: 191, and a VL that is a VL in SEQ ID NO: 191. In some embodiments is a fusion protein comprising at least one TRAF variant of any one of the embodiments of the present disclosure. In some embodiments, the fusion protein further comprises a first sequence, comprising any one or more of: a HCDR1 that is an HCDR1 in SEQ ID NO: 186, a HCDR2 that is an HCDR2 in SEQ ID NO: 186, a HCDR3 that is an HCDR3 in SEQ ID NO: 186, a LCDR1 that is an LCDR1 in SEQ ID NO: 186, a LCDR2 that is an LCDR2 in SEQ ID NO: 186, and LCDR3 that is an LCDR3 in SEQ ID NO: 186; a HCDR1 that is an HCDR1 in SEQ ID NO: 187, a HCDR2 that is an HCDR2 in SEQ ID NO: 187, a HCDR3 that is an HCDR3 in SEQ ID NO: 187, a LCDR1 that is an LCDR1 in SEQ ID NO: 187, a LCDR2 that is an LCDR2 in SEQ ID NO: 187, and LCDR3 that is an LCDR3 in SEQ ID NO: 187; a HCDR1 that is an HCDR1 in SEQ ID NO: 188, a HCDR2 that is an HCDR2 in SEQ ID NO: 188, a HCDR3 that is an HCDR3 in SEQ ID NO: 188, a LCDR1 that is an LCDR1 in SEQ ID NO: 188, a LCDR2 that is an LCDR2 in SEQ ID NO: 188, and LCDR3 that is an LCDR3 in SEQ ID NO: 188; a HCDR1 that is an HCDR1 in SEQ ID NO: 189, a HCDR2 that is an HCDR2 in SEQ ID NO: 189, a HCDR3 that is an HCDR3 in SEQ ID NO: 189, a LCDR1 that is an LCDR1 in SEQ ID NO: 189, a LCDR2 that is an LCDR2 in SEQ ID NO: 189, and LCDR3 that is an LCDR3 in SEQ ID NO: 189; a HCDR1 that is an HCDR1 in SEQ ID NO: 190, a HCDR2 that is an HCDR2 in SEQ ID NO: 190, a HCDR3 that is an HCDR3 in SEQ ID NO: 190, a LCDR1 that is an LCDR1 in SEQ ID NO: 190, a LCDR2 that is an LCDR2 in SEQ ID NO: 190, and LCDR3 that is an LCDR3 in SEQ ID NO: 190; a HCDR1 that is an HCDR1 in SEQ ID NO: 191, a HCDR2 that is an HCDR2 in SEQ ID NO: 191, a HCDR3 that is an HCDR3 in SEQ ID NO: 191, a LCDR1 that is an LCDR1 in SEQ ID NO: 191, a LCDR2 that is an LCDR2 in SEQ ID NO: 191, and LCDR3 that is an LCDR3 in SEQ ID NO: 191; a VH that is a VH in SEQ ID NO: 186, and a VL that is a VL in SEQ ID NO: 186; a VH that is a VH in SEQ ID NO: 187, and a VL that is a VL in SEQ ID NO: 187; a VH that is a VH in SEQ ID NO: 188, and a VL that is a VL in SEQ ID NO: 188; a VH that is a VH in SEQ ID NO: 189, and a VL that is a VL in SEQ ID NO: 189; a VH that is a VH in SEQ ID NO: 190, and a VL that is a VL in SEQ ID NO: 190; or a VH that is a VH in SEQ ID NO: 191, and a VL that is a VL in SEQ ID NO: 191. In some embodiments, the fusion protein further comprises a second sequence that is a transmembrane domain, a third sequence that comprises the amino acid sequence of SEQ ID NO: 25; and a fourth sequence that comprises the amino acid sequence of SEQ ID NO: 32, wherein the first sequence is linked to the second sequence, wherein the second sequence is linked to the third sequence, and wherein the third sequence is linked to the fourth sequence. In some embodiments, any of the CD40, TRAF2, TRAF2/3, or TRAF6 variants provided herein, including those in FIGS. 28, 67, 77A-77C, 83-89D, and 92 (excluding those in FIGS. 78 and 79), and including those in Tables 2, 4-5, and 7-14, and including SEQ ID NOS: 42, 475-479, 494-498, 505, 510-519, and 840-40839, and Consensus sequences A-C, can be used in the present method and/or construct and/or protein. The sequence options in FIGS. 78 and 79 can be optionally excluded from all of the embodiments provided herein.

In some embodiments is a CoStAR comprising at least one TRAF variant of any one of the embodiments of the present disclosure, wherein the CoStAR is capable of CoStAR-ligand-inducible activation of an immune cell bearing a T cell receptor (TCR) when the TCR is engaged by a target of the TCR, which comprises an extracellular ligand binding domain specific for the inducing ligand, a transmembrane domain, and an intracellular signaling domain. In some embodiments is a cell expressing the CoStAR, antibody, or protein of any one of the embodiments of the present disclosure. In some embodiments, the cell comprises an alpha-beta T cell. In some embodiments, the alpha-beta T cell comprises a tumor infiltrating lymphocyte (TIL). In some embodiments the CoStAR does not comprise a full length CD28 extracellular domain. In some embodiments the CoStAR comprises a truncated CD28 extracellular domain or a CD8 extracellular domain located between the ligand binding domain and the transmembrane domain. In some embodiments, the intracellular signaling domain comprises one or more of CD28, CD40, CD137, OX40, ICOS, CD2, or DAP10 or signaling fragments thereof. In some embodiments, the ligand-binding domain binds to tafasitamab, cetuximab, trastuzumab, and/or pembrolizumab. In some embodiments, any of the CD40, TRAF2, TRAF2/3, or TRAF6 variants provided herein, including those in FIGS. 28, 67, 77A-77C, 83-89D, and 92 (excluding those in FIGS. 78 and 79), and including those in Tables 2, 4-5, and 7-14, and including SEQ ID NOS: 42, 475-479, 494-498, 505, 510-519, and 840-40839, and Consensus sequences A-C, can be used in the present method and/or construct and/or protein. The sequence options in FIGS. 78 and 79 can be optionally excluded from all of the embodiments provided herein.

In some embodiments is a method of cell therapy, the method comprising identifying a subject in need thereof and administering the CoStAR, fusion protein, nucleic acid, vector, or cell of any one of the embodiments of the present disclosure.

In some embodiments is a method for preparing a population of cells enriched for the CoStAR or protein expressing the TRAF variant, the method comprising detecting expression of the CoStAR or protein on the surface of the cells transfected or transduced with a vector of any one of the embodiments of the present disclosure and selecting cells with are identified as expressing the CoStAR or protein.

In some embodiments is a method for treating a disease in a subject in need thereof, the method comprising administering the cell or cell population of any one of the embodiments of the present disclosure to the subject.

### CD40 and TRAF

CD40 is a TNF receptor superfamily member that provides activation signals in antigen-presenting cells such as B cells, macrophages, and dendritic cells. Multimerization of CD40 by its ligand initiates signaling by recruiting TNF receptor-associated factors (TRAFs) to the CD40 cytoplasmic domain. TRAF binding sites are responsible for the intracellular signalling of CD40.

The TRAF domain mediates oligomerization of TRAF proteins as well as their association with upstream receptors or adaptors and downstream effector proteins. The RING domain is best known for its function to mediate protein ubiquitination in a large family of E3 ubiquitinase ligases. Among the major downstream pathways regulated by TRAFs are those leading to activation of the nuclear factor kappa beta (NF-κB) and mitogen-activated protein kinases (MAPKs), which are in turn important for induction of genes associated with innate immunity, inflammation, and cell survival. TRAF2 and TRAF3, function as negative regulators in some signaling pathways involved in the survival of B cells and inflammatory responses of innate immune cells.

The canonical pathway of NF-κB is induced by TNFα, IL-1, or LPS and uses a large variety of signalling adaptors to engage IKK activity. Phosphorylation of serine residues in the signal responsive region (SRR) of classical IκBs by IKKβ leads to IκB ubiquitination and subsequent proteosomal degradation. This results in release of the NF-κB dimer, which can then translocate to the nucleus and induce transcription of target genes. The non-canonical pathway depends on NIK (NF-κB-inducing kinase) induced activation of IKKα. IKKα phosphorylates the p100 NF-κB subunit, which leads to proteosomal processing of p100 to p52. This results in the activation of p52-RelB dimers, which target specific κB elements.

It has been discovered that costimulatory receptors comprising a CD40 signaling domain display novel and improved activity profiles. It has further been discovered that variants of TRAF2, TRAF2/3, and TRAF6 binding domains within CD40 modulate signaling. The activity profiles can be modulated by selecting an intracellular domain of a receptor protein for joining to the CD40 signaling domain and/or by selecting elements of the CD40 signaling domains to join to the intracellular domain of a receptor protein. Provided herein are recombinant costimulatory antigen receptors (CoStARs) comprising: (i) a disease- or tumor-associated antigen binding domain, (ii) a first intracellular segment comprising an intracellular signaling domain of a receptor protein, and (iii) a second intracellular signaling domain of a CD40 receptor protein or signal transducing fragment thereof. Optionally, the CoStAR comprises an extracellular segment of a stimulatory receptor protein. In some embodiments, the extracellular segment of the stimulatory receptor protein is capable of binding ligand. In some embodiments, the extracellular segment of a stimulatory receptor protein is truncated and does not bind ligand. In some embodiments the extracellular segment of the stimulatory receptor protein operates as an adjustable length spacer allowing the disease- or tumor-associated antigen binding domain to be located away from the surface of the cell in which it is expressed for example to form a more optimal immune synapse. In some embodiments, the extracellular segment of a stimulatory receptor protein and the first intracellular segment comprise segments of the same receptor protein. In some embodiments, the extracellular segment and the first intracellular segment comprise segments of different receptor proteins. The CoStARs comprise an intervening transmembrane domain between the disease or tumor antigen binding domain and the first intracellular domain. When an extracellular segment of a stimulatory receptor protein is present, the transmembrane domain is intervening between the extracellular segment and the first intracellular signaling domain. In some embodiments, any of the CD40, TRAF2, TRAF2/3, or TRAF6 variants provided herein, including those in FIGS. 28, 67, 77A-77C, 83-89D, and 92 (excluding those in FIGS. 78 and 79), and including those in Tables 2, 4-5, and 7-14, and including SEQ ID NOS: 42, 475-479, 494-498, 505, 510-519, and 840-40839, and Consensus sequences A-C, can be used in the present methods and/or constructs and/or proteins. The sequence options in FIGS. 78 and 79 can be optionally excluded from all of the embodiments provided herein.

As used herein, “full length protein” or “full length receptor” refers to a receptor protein, such as, for example, a CD28 receptor protein. The term “full length” encompasses receptor proteins lacking up to about 5 or up to 10 amino acids, for example 1, 2, 3, 4, 5, 6, 7, 8, 9, or 10 amino acids, at the N-terminal of the mature receptor protein once its signal peptide has been cleaved. For instance, while a specific cleavage site of a receptors N-terminal signal peptide may be defined, variability in exact point of cleavage has been observed. The term “full length” does not imply presence or absence of amino acids of the receptors N-terminal signal peptide. In one embodiment, the term “full length” (e.g. a full length CD28 or a full length CD40 intracellular domain, according to certain aspects of the invention) encompasses mature receptor proteins (e.g. CD28 according to certain aspects of the invention) lacking the N terminal signal peptide lacking up to about 5, for example 1, 2, 3, 4, 5, or up to 10 amino acids at the N-terminal of the mature receptor protein once its signal peptide has been cleaved. As mentioned above, a “full length” CD28 receptor or other receptor or tumor antigen binding domain according to the various aspects of the invention does not include the signal peptide and may lack up to about 5, for example 1, 2, 3, 4, 5, or up to 10 amino acids at the N-terminal of the mature receptor protein (e.g. N terminal residues N, K, I, L and/or V). This is shown in the exemplary fusions, e.g. SEQ ID Nos. 4-12 (note that these may lack up to about 5, for example 1, 2, 3, 4, 5, or up to 10 amino acids at the N-terminal of the mature receptor protein as shown in the boxed region).

CoStARs have modular form and can be constructed to comprise extracellular, transmembrane and intracellular domains obtained from a one or more proteins, along with the scFv obtained from an antibody that binds to a disease-associated antigen, for example, a tumor associated antigen.

According to the invention, in one embodiment, a CoStAR comprises a disease-associated, for example a tumor-associated, antigen receptor, such as but not limited to a tumor-associated antigen specific scFv, and a primary costimulatory receptor protein that is capable of binding to its cognate ligand and providing an intracellular signal. In some embodiments, the primary costimulatory receptor can be less than a full length protein but is sufficient to bind cognate ligand and transduce a signal. In some embodiments, the primary costimulatory receptor domain is full length, such as but not limited to, full length CD28. Thus, both the antigen specific binding domain and the ligand specific receptor are capable of binding cognate antigen and ligand respectively. The amino acid sequences provided herein provide embodiments of several CoStAR constructs. These include CoStARs constructs that comprise an antigen binding domain, an optional spacer, an optional costimulatory receptor protein comprising an extracellular ligand binding segment or fragment thereof and intracellular CD40 signaling domain. In another embodiment, a CoStAR comprises an antigen binding domain, an optional spacer, an extracellular ligand-binding portion of a costimulatory receptor protein, a transmembrane domain, and an intracellular signaling domain of a selected costimulatory receptor protein and intracellular CD40 signaling domain. In some embodiments, the extracellular ligand-binding portion comprises a CD28 truncation, for example, a C-terminal CD28 truncation after amino acids IEV, and is followed by an intracellular signaling domain. In some embodiments, the intracellular signaling domain is from CD40. The transmembrane domain separating the extracellular ligand-binding and intracellular signaling domains can be from, with limitation, CD28, CD40. In further embodiments, CoStARs can comprise additional costimulatory domains, for example a third, intracellular costimulatory signaling domain and in this respect may be similar to certain chimeric antigen receptors (CARs), which have been classified into first (CD3ζ only), second (one costimulatory domain+CD3ζ), or third generation (more than one costimulatory domain+CD3ζ).

Costimulatory receptor proteins useful in CoStARs of the invention include, without limitation, CD2, CD9, CD26, CD27, CD28, CD29, CD38, CD40, CD43, CD46, CD49d, CD55, CD73, CD81, CD82, CD99, CD100, CD134 (OX40), CD137 (41BB), CD150 (SLAM), CD270 (HVEM), CD278 (ICOS), CD357 (GITR), or EphB6, which in their natural form comprise extracellular ligand binding domains and intracellular signal transducing domains. For example, CD2 is characterized as a cell adhesion molecule found on the surface of T cells and is capable of initiating intracellular signals necessary for T cell activation. CD27 is characterized as a type II transmembrane glycoprotein belonging to the TNFR superfamily (TNFRSF) whose expression on B cells is induced by antigen-receptor activation in B cells. CD28 is one of the proteins on T cells and is the receptor for CD80 (B7.1) and CD86 (B7.2) ligands on antigen-presenting cells. CD137 (4-1BB) ligand is found on most leukocytes and on some non-immune cells. OX40 ligand is expressed on many antigen-presenting cells such as DC2s (dendritic cells), macrophages, and B lymphocytes. In one embodiment, the costimulatory receptor protein is full length CD28 as defined herein.

CD40 is a member of the tumor necrosis factor receptor (TNFR) superfamily and several isoforms are generated by alternative splicing. Its ligand, CD154 (also called CD40L) is a protein that is primarily expressed on activated T cells. For reference, the human CD40 isoform 1 protein sequence is set forth in GenBank accession No. NP_001241.1, including signal peptide (amino acids 1-20), transmembrane domain (amino acids 194-215), and cytoplasmic domain (amino acids 216-277)(SEQ ID NO:32). CD40 receptor signaling involves adaptor proteins including but not limited to TNF receptor-associated factors (TRAF), and the CD40 cytoplasmic domain comprises signaling components, including amino acid sequences fitting an SH3 motif (KPTNKAPH) (SEQ ID NO:35), TRAF2 motif (PKQE (SEQ ID NO:36), PVQE (SEQ ID NO:37), SVQE (SEQ ID NO:38)), TRAF6 motif (QEPQEINFP) (SEQ ID NO:39) and PKA motif (KKPTNKA (SEQ ID NO:40), SRISVQE (SEQ ID NO:41)). The invention further includes engineered signaling domains, such as engineered CD40 signaling domains, comprising TRAF-binding amino acid sequences. Engineered signaling domains that bind to TRAF1, TRAF2, TRAF3, and TRAF5 may comprise the major consensus sequence (P/S/A/T)X(Q/E)E or minor consensus sequence PXQXXD and can be identified in or obtained from, without limitation, TNFR family members such as CD30, OX40, 41BB, and the EBV oncoprotein LMP1. (See, e.g., Ye, H et al., The Structural Basis for the Recognition of Diverse Receptor Sequences by TRAF2. Molecular Cell, 1999; 4(3):321-30. doi: 10.1016/51097-2765(00)80334-2; Park H H, Structure of TRAF Family: Current Understanding of Receptor Recognition. Front. Immunol. 2018; 9:1999. doi: 10.3389/fimmu.2018.01999; Chung, J. Y. et al., All TRAFs are not created equal: common and distinct molecular mechanisms of TRAF-mediated signal transduction. Journal of Cell Science 2002; 115:679-688).

Examples disclosed herein demonstrate operation of CD40 as a costimulatory signaling domain in a CoStAR and further that cytokine and chemokine expression profiles are altered by signaling domain selection. In some embodiments, the costimulatory CD40 signaling domain of a CoStAR promotes pro-inflammatory cytokines (e.g., IL-2, TNFα). In some embodiments, the costimulatory CD40 signaling domain of a CoStAR reduces immunosuppressive cytokines (e.g., IL-5, IL-10). Costimulatory activity of a CD40 signaling domain or fragment can be observed in combination with a first receptor signaling domain such as but not limited to CD2, CD9, CD26, CD27, CD28, CD29, CD38, CD40, CD43, CD46, CD49d, CD55, CD73, CD81, CD82, CD99, CD100, CD134 (OX40), CD137 (41BB), CD150 (SLAM), CD270 (HVEM), CD278 (ICOS), CD357 (GITR), or EphB6, as compared to activity of the first receptor signaling domain without the CD40 signaling domain or fragment. In this regard, the CD40 signaling domains of the invention, including signaling fragments comprising particular factor binding sites or wherein particular factor binding sites are mutated, in combination with a costimulatory first signaling domain, are capable of promoting or suppressing relative expression of particular cytokines and/or chemokines as compared to the first signaling domain alone. activity of a costimulatory signaling domain. (See, e.g., Ahonen, C L et al., The CD40-TRAF6 axis controls affinity maturation and the generation of long-lived plasma cells. Nat Immunol. 2002; 3: 451-456; Mackey M F et al., Distinct contributions of different CD40 TRAF binding sites to CD154-induced dendritic cell maturation and IL-12 secretion. Eur J Immunol. 2003; 33: 779-789; Mukundan L et al., TNF receptor-associated factor 6 is an essential mediator of CD40-activated proinflammatory pathways in monocytes and macrophages. J Immunol. 2005; 174: 1081-1090.

In some embodiments, a CoStAR of the invention comprises substantially all of a CD40 costimulatory domain. In some embodiments, a CoStAR of the invention comprises two or more CD40 costimulatory domains. In some embodiments, a CoStAR of the invention comprises a CD40 costimulatory domain signaling component or fragment or motif. In some embodiments, the CD40 signaling fragment or motif comprises, consists, or consists essentially of an SH3 binding sequence (e.g., without limitation, KPTNKAPH (SEQ ID NO:35), PTNKAPHP (SEQ ID NO:443) or PTNKAPH (SEQ ID NO:444)), TRAF2/TRAF3 binding sequence (e.g., without limitation, PKQE (SEQ ID NO:506), PKQET (SEQ ID NO:445), PVQE (SEQ ID NO:507), PVQET (SEQ ID NO:446), SVQE (SEQ ID NO:508), SVQET (SEQ ID NO:447)), TRAF6 binding sequence (e.g., without limitation, PQEINF (SEQ ID NO:509), QEPQEINF (SEQ ID NO:448) or QEPQEINFP (SEQ ID NO:39)) or PKA sequence (e.g., without limitation, KKPTNKA (SEQ ID NO:40), or SRISVQE (SEQ ID NO:41) as well as two or more, or three or more, or four or more such components or motifs, or combinations thereof, which can be in multiple copies and arranged in any order. In some embodiments, a CoStAR of the invention comprises a CD40 costimulatory domain and a CD40 costimulatory domain signaling component or motif. In some embodiments, one or more of the SH3, TRAF2/TRAF3, TRAF6, or PKA motifs of the CD40 signaling domain is mutated. In some embodiments, the SH3 motif, TRAF2/TRAF3 motif, and TRAF6 motif are sufficient to modulate pro-inflammatory and/or immunosuppressive cytokines. In some embodiments, adding tandem copies of those motifs and/or mutating certain motifs amplifies these effects.

Table 1 provides non-limiting examples of TRAF2/TRAF3 binding domains. The alignments of Table 1 with the minor and major consensus sequences are as shown in FIGS. 82A-82B, respectively.

TABLE 1
Exemplary TRAF2/TRAF3 binding sequences

| | | | | | | | | | | | | SEQ |
|---|---|---|---|---|---|---|---|---|---|---|---|---|
| | | | | | | | | | | | | ID |
| Source | P-4 | P-3 | P-2 | P-1 | P0 | P1 | P2 | P3 | P4 | P5 | P6 | NO. |
| hTNFR2 | P | F | S | K | E | E | C | A | F | R | S | 450 |
| hCD40 | A | A | P | V | Q | E | T | L | H | G | C | 451 |
| hCD30 | M | L | S | V | E | E | E | G | K | E | D | 452 |
| hCD27 | T | I | P | I | Q | E | D | Y | R | K | P | 453 |
| hLTβR | S | T | P | H | Q | E | D | G | K | A | W | 454 |
| hATAR | T | V | A | V | E | E | T | I | P | S | T | 455 |
| hOX40 | R | T | P | I | Q | E | E | Q | A | D | A | 456 |
| m41BB | T | G | A | A | Q | E | E | D | A | C | S | 457 |
| m41BB | R | C | P | Q | E | E | E | G | G | G | G | 458 |
| h41BB | V | Q | T | T | Q | E | E | D | G | C | S | 459 |
| h41BB | R | F | P | E | E | E | E | G | G | C | E | 460 |
| bLMP1 | R | T | P | V | Q | E | S | G | Y | P | D | 461 |
| bLMP1 | R | P | P | V | Q | E | T | G | G | G | G | 462 |
| bLMP1 | H | P | P | V | Q | E | T | G | G | G | G | 463 |
| bLMP1 | H | P | P | V | Q | E | T | G | E | G | G | 464 |
| bLMP1 | H | P | P | I | Q | E | T | G | N | G | G | 465 |
| LAT | A | L | S | S | Q | E | A | E | E | V | E | 466 |
| hTANK | S | V | P | I | Q | C | T | D | K | T | D | 467 |
| hLMP1 | P | H | P | Q | Q | A | T | D | D | S | S | 468 |
| rLMP1 | P | Y | P | I | Q | A | T | D | G | G | N | 469 |
| rLMP1 | P | H | P | I | Q | A | T | D | G | A | N | 470 |
| rLMP1 | P | Y | P | V | Q | A | S | D | G | G | D | 471 |
| Minor | | | P/S/ | X | Q/E | E | | | | | | 472 |
| Consensus | | | A/T | | | | | | | | | |
| Major | | | P | V | Q | E | | | | | | 473 |
| Consensus | | | | | | | | | | | | |

Accordingly, TRAF2/TRAF3 binding sequences of the invention further include sequences such as P1V₂Q₃E₄ and variants wherein P₁ is substituted with S, A, or T, V₂ is substituted with Q, K, or E, Q₃ is substituted with E, and/or E4 is substituted with A. In such variants, any one, two, three, or all four of P₁V₂Q₃E₄ may be substituted. Non-limiting examples are shown in Table 1 at positions P-2, P-1, P0, P1.

Illustrative non-limiting examples of CD40 TRAF2/TRAF3 sequence variants include the following, the amino acids at P-2, P-1, P0, and P1 enclosed by dashes, and the TRAF2/TRAF3 source protein identified.

TABLE 2
Exemplary CD40 TRAF2/TRAF3 variants

| | | TRAF2/3 |
|---|---|---|
| SEQ ID | | sequence |
| NO: | Amino acid sequence based on CD40 | origin |
| 474 | KKVAKKPTNKAPHPKQEPQEINFPDDLPGSNTAA--PVQE-- | CD40 WT |
| | TLHGCQPVTQEDGKESRI--SVQE--RQ | |
| 475 | KKVAKKPTNKAPHPKQEPQEINFPDDLPGSNTAA--TQEE-- | CD40/ |
| | TLHGCQPVTQEDGKESRISVQERQ | v41BB |
| 476 | KKVAKKPTNKAPHPKQEPQEINFPDDLPGSNTAA--SKEE-- | CD40/ |
| | TLHGCQPVTQEDGKESRISVQERQ | VTNFR2 |
| 477 | KKVAKKPTNKAPHPKQEPQEINFPDDLPGSNTAA--AVEE-- | CD40/ |
| | TLHGCQPVTQEDGKESRISVQERQ | vATAR |
| 478 | KKVAKKPTNKAPHPKQEPQEINFPDDLPGSNTAAPVQE | CD40/ |
| | TLHGCQPVTQEDGKESRI--AVEE--RQ | vATAR |
| 479 | KKVAKKPTNKAPHPKQEPQEINFPDDLPGSNTAAPVQE | CD40/ |
| | TLHGCQPVTQEDGKESRI--PEEE--RQ | v41BB |

Table 3 provides non-limiting examples of TRAF6 binding sequences. The alignments of Table 3 with the major TRAF6 consensus sequences is as shown in FIG. 82C.

TABLE 3
Exemplary TRAF6 binding sequences

| | | | | | | | SEQ |
|---|---|---|---|---|---|---|---|
| | | | | | | | ID |
| | P-2 | P-1 | P0 | P1 | P2 | P3 | NO: |
| hCD40 | P | Q | E | I | N | F | 480 |
| hTRANCE-R | P | Q | E | I | D | F | 481 |
| Mal | P | P | E | L | R | F | 482 |
| TRIF | P | E | E | M | S | W | 483 |
| IRAK(1) | P | Q | E | N | S | Y | 484 |
| IRAK (2) | P | V | E | S | D | E | 485 |
| IRAK (3) | P | E | E | S | D | E | 486 |
| IRAK-2 (1) | P | E | E | T | D | E | 487 |
| IRAK-2 (2) | P | T | E | N | G | E | 488 |
| IRAK-M | P | V | E | D | D | E | 489 |
| RIP2 | P | P | E | N | Y | E | 490 |
| MyD88 | P | S | E | L | R | F | 491 |
| Consensus | P | X | E | X | X | Ac/Ar | 492 |

Ac = acidic residue; Ar = Aromatic residue

Illustrative non-limiting examples of CD40 TRAF6 sequence variants include the following, the amino acids at P-2, P-1, P0, P1, P2, and P3 enclosed by dashes, and the TRAF6 sequence origin identified.

TABLE 4
Exemplary CD40 TRAF6 variants

| | | TRAF6 |
|---|---|---|
| SEQ ID | | sequence |
| No: | Amino acid sequence based on CD40 | origin |
| 493 | KKVAKKPTNKAPHPKQE--PQEINF--PDDLPGSNTAAPVQE | CD40 WT |
| | TLHGCQPVTQEDGKESRISVQERQ | |
| 494 | KKVAKKPTNKAPHPKQE--PEEMSW--PDDLPGSNTAAPVQE | CD40/ |
| | TLHGCQPVTQEDGKESRISVQERQ | vTRIF |
| 495 | KKVAKKPTNKAPHPKQE--PPENYE--PDDLPGSNTAAPVQE | CD40/ |
| | TLHGCQPVTQEDGKESRISVQERQ | vRIP2 |
| 496 | KKVAKKPTNKAPHPKQE--PQENSY--PDDLPGSNTAAPVQE | CD40/ |
| | TLHGCQPVTQEDGKESRISVQERQ | vIRAK(1) |

TABLE 5
Exemplary CD40 TRAF2/TRAF3/TRAF6 consensus variants

| SEQ ID | | |
|---|---|---|
| No: | Amino acid sequence variants based on CD40 | |
| 497 | KKVAKKPTNKAPHPKQE--PQEINF--PDDLPGSNTAA--PVQE-- | CD40 |
| | TLHGCQPVTQEDGKESRI--SVQE--RQ | WT |
| 498 | KKVAKKPTNKAPHPKQE--PXEXX(Ac/Ar)-- | |
| | PDDLPGSNTAAPVQETLHGCQPVTQEDGKESRISVQERQ | |
| 499 | KKVAKKPTNKAPHPKQEPQEINFPDDLPGSNTAA-- | |
| | (P/S/A/T)X(Q/E)E--TLHGCQPVTQEDGKESRISVQERQ | |
| 500 | KKVAKKPTNKAPHPKQEPQEINFPDDLPGSNTAAPVQE | |
| | TLHGCQPVTQEDGKESRI--(P/S/A/T)X(Q/E)E--RQ | |
| 501 | KKVAKKPTNKAPHPKQE--PXEXX(Ac/Ar)--PDDLPGSNTAA-- | |
| | (P/S/A/T)X(Q/E)--ETLHGCQPVTQEDGKESRISVQERQ | |
| 502 | KKVAKKPTNKAPHPKQEPQEINFPDDLPGSNTAA-- | |
| | (P/S/A/T)X(Q/E)E--TLHGCQPVTQEDGKESRI-- | |
| | (P/S/A/T)X(Q/E)E--RQ | |
| 503 | KKVAKKPTNKAPHPKQE--PXEXX(Ac/Ar)-- | |
| | PDDLPGSNTAAPVQE | |
| | TLHGCQPVTQEDGKESRI--(P/S/A/T)X(Q/E)E--RQ | |
| 504 | KKVAKKPTNKAPHPKQE--PXEXX(Ac/Ar)--PDDLPGSNTAA-- | |
| | (P/S/A/T)X(Q/E)E--TLHGCQPVTQEDGKESRI-- | |
| | (P/S/A/T)X(Q/E)E--RQ | |
| 505 | KKVAKKPTNKAPHPKQE--PEELRW--PDDLPGSNTAA--THEE-- | |
| | TLHGCQPVTQEDGKESRI--AIEE--RQ | |

Ar = aromatic residue; Ac = acidic residue

In some embodiments, selection of one or more costimulatory domain signaling component or motif is guided by the cell in which the CoStAR is to be expressed and/or a desired costimulatory activity more closely identified with a signaling component or motif, or avoidance of a costimulatory activity more closely identified with a signaling component or motif.

In some embodiments, a CoStAR signaling domain comprises, in addition to a CD40 costimulatory domain or signaling component or motif thereof, or two or more such domains or components or motifs or combinations thereof, an additional full length costimulatory domain or signaling component thereof from, without limitation, CD2, CD9, CD26, CD27, CD28, CD29, CD38, CD40, CD43, CD46, CD49d, CD55, CD73, CD81, CD82, CD99, CD100, CD134 (OX40), CD137 (41BB), CD150 (SLAM), CD270 (HVEM), CD278 (ICOS), CD357 (GITR), or EphB6,

For reference, the human CD28 protein sequence is set forth in GenBank accession No. NP_006130.1, including signal peptide (amino acids 1-18), extracellular domain (amino acids 19-152), transmembrane domain (amino acids 153-179) and cytoplasmic domain (amino acids 180-200). The extracellular domain includes an immunoglobulin type domain (amino acids 21-136) which contains amino acids with compose the antigen binding site and amino acids that form the homodimer interface. The extracellular domain includes several asparagine residues which may be glycosylated, and the intracellular domain comprises serine and tyrosine residues, which may be phosphorylated.

For reference, the human CD8 alpha chain protein sequence is set forth by GenBank accession No. NP_001139345.1, including signal peptide (amino acids 1-21), extracellular domain (amino acids 22-182), transmembrane domain (amino acids 183-203), and cytoplasmic domain (amino acids 204-235). The extracellular domain includes an immunoglobulin type domain (amino acids 28-128) which contains amino acids with compose the antigen binding site and amino acids that form the homodimer interface. The extracellular domain includes several asparagine residues which may be glycosylated, and the intracellular domain comprises serine and tyrosine residues, which may be phosphorylated.

For reference, the human IgG4 constant region sequence is set forth in UniProtKB/Swiss-Prot: accession No. P01861.1, including CH1 (amino acids 1-98), hinge (amino acids 99-110), CH2 (amino acids 111-220), CH3 (amino acids 221-327). The CH2 region includes asparagine at amino acid 177, which is the glycosylated and associated with Fc receptor and antibody-dependent cell-mediated cytotoxicity (ADCC).

For reference, the protein sequence of human CD137 (4-1BB), another TNFR superfamily member, is set forth by GenBank accession No. NP_001552.2, including signal peptide (amino acids 1-23), extracellular domain (amino acids 24-186), transmembrane domain (amino acids 187-213), and cytoplasmic domain (amino acids 214-255). Binding of CD137L ligand trimers expressed on antigen presenting cells to CD137 leads to receptor trimerization and activation of signaling cascades involved in T cell reactivity and survival (Li et al., Limited Cross-Linking of 4-1BB by 4-1BB Ligand and the Agonist Monoclonal Antibody Utomilumab. Cell Reports 2018; 25:909-920). Coimmunoprecipitation of CD137 with the signaling adaptors TRAF-2 and TRAF-1 and the structural basis for the interactions has been reported (Ye, H et al., Molecular Cell, 1999; 4(3):321-30).

For reference, the human CD134 (OX40) protein sequence is set forth by GenBank accession No. NP_003318.1, including signal peptide (amino acids 1-28), extracellular domain (amino acids 29-214), transmembrane domain (amino acids 215-235), and cytoplasmic domain (amino acids 236-277). This receptor has been shown to activate NF-kappaB through its interaction with adaptor proteins TRAF2 and TRAF5 and studies suggest that this receptor promotes expression of apoptosis inhibitors BCL2 and BCL21L1/BCL2-XL.

The human T-cell surface antigen CD2 has at least two isoforms. For reference, the human CD2 isoform1 protein sequence is set forth by NP_001315538.1, including signal peptide (amino acids 1-24), extracellular domain (amino acids 25-235), transmembrane domain (amino acids 236-261), and cytoplasmic domain (amino acids 262-377). The human CD2 isoform2 protein sequence is set forth by NP_001758.2

For reference, the human CD357 (GITR) isoform-1 protein sequence is set forth by GenBank accession No. NP_004186.1, including signal peptide (amino acids 1-25), extracellular domain (amino acids 26-162), transmembrane domain (amino acids 163-183), and cytoplasmic domain (amino acids 184-241).

For reference, the human CD29 (beta1 integrin) protein sequence is set forth by GenBank accession No. NP_596867, including signal peptide (amino acids 1-20), extracellular domain (amino acids 21-728), transmembrane domain (amino acids 729-751), and cytoplasmic domain (amino acids 752-798).

The human CD150 (SLAM) protein sequence has at several isoforms. In addition to the transmembrane form of CD150 (mCD150), cells of hematopoietic lineage express mRNA encoding the secreted form of CD150 (sCD150), which lacks the entire transmembrane region of 30 amino acids. For reference, human SLAM isoform b is set forth by GenBank accession No. NP_003028.1, including signal peptide (amino acids 1-20), extracellular domain (amino acids 21-237), transmembrane domain (amino acids 238-258), and cytoplasmic domain (amino acids 259-335). Human SLAM isoform a is set forth by GenBank accession No. NP_001317683.1.

CD278 or ICOS (Inducible T cell COStimulator) is a CD28-superfamily costimulatory molecule that is expressed on activated T cells. Human ICOS precursor (199 aa with signal peptide) is set forth by GenBank accession No. NP_036224.1, including signal peptide (amino acids 1-20), an Ig-V-like domain (amino acids 21-140), transmembrane domain (amino acids 141-161) and intracellular domain (amino acids 162-199). ICOS contains an IProx motif sequence SSSVHDPNGE (SEQ ID NO:466). The IProx motif sequence SSSXXXPXGE (SEQ ID NO:467) resembles certain binding sites of TRAF1 (SASFQRPQSE (SEQ ID NO:468)), TRAF2 (SSSFQRPVND (SEQ ID NO:469)), TRAF3 (SSFKKPTGE (SEQ ID NO:470)), and TRAF5 (SSSFKRPDGE (SEQ ID NO:471)).

Hematopoietic cell signal transducer (HCST) also known as DAP10, KAP10, PIK3AP, and hematopoietic cell signal transducer (GenBank accession No. NP_055081.1) encodes a transmembrane signaling adaptor thought to form part of a receptor complex with the C-type lectin-like receptor NKG2D. The intracellular domain contains a YxxM motif of a phosphatidylinositol 3-kinase binding site.

In embodiments of the invention, a CoStAR may be expressed alone under the control of a promoter in a therapeutic population of cells that have therapeutic activity, for example, Tumour Infiltrating Lymphocytes (TILs). Alternatively, the CoStAR may be expressed along with a therapeutic transgene such as a chimeric antigen receptor (CAR) and/or T-cell Receptor (TCR), (note that may lack up to about 5, for example 1, 2, 3, 4, 5, or up to 10 amino acids at the N-terminal of the mature receptor protein). Thus, in one aspect, the invention also relates to CoStAR constructs, not limited to those having a sequence as shown in any of SEQ ID NOS:42-185, 192-335, 344-430, including one of these sequences which lacks up to about 5, for example 1, 2, 3, 4, 5, or up to 10 amino acids at the N-terminal of the mature receptor protein). Suitable TCRs and CARs are well known in the literature, for example HLA-A*02-NYESO-1 specific TCRs (Rapoport et al. Nat Med 2015) or anti-CD19scFv.CD3ζ fusion CARs (Kochenderfer et al. J Clin Oncol 2015) which have been successfully used to treat Myeloma or B-cell malignancies respectively. The CoStARs described herein may be expressed with any known CAR or TCR thus providing the cell with a regulatable growth switch to allow cell expansion in-vitro or in-vivo, and a conventional activation mechanism in the form of the TCR or CAR for anti-cancer activity. Thus the invention provides a cell for use in adoptive cell therapy comprising a CoStAR as described herein and a TCR and/or CAR that specifically binds to a tumor associated antigen. An exemplary CoStAR comprising CD28 includes an extracellular antigen binding domain and an extracellular, transmembrane and intracellular signaling domain.

The term “antigen binding domain” as used herein refers to an antibody fragment including, but not limited to, a diabody, a Fab, a Fab′, a F(ab′)2, an Fv fragment, a disulfide stabilized Fv fragment (dsFv), a (dsFv)2, a bispecific dsFv (dsFv-dsFv′), a disulfide stabilized diabody (ds diabody), a single-chain antibody molecule (scFv), an scFv dimer (bivalent diabody), a multispecific antibody formed from a portion of an antibody comprising one or more CDRs, a camelized single domain antibody, a nanobody, a domain antibody, a bivalent domain antibody, or any other antibody fragment that binds to an antigen but does not comprise a complete antibody structure. An antigen binding domain is capable of binding to the same antigen to which the parent antibody or a parent antibody fragment (e.g., a parent scFv) binds. In some embodiments, an antigen-binding fragment may comprise one or more complementarity determining regions (CDRs) from a particular human antibody grafted to frameworks (FRs) from one or more different human antibodies. An “antigen binding domain” may be referred to as a “ligand binding domain.”

The antigen binding domain can be made specific for any disease-associated antigen, including but not limited to tumor-associated antigens (TAAs) and infectious disease-associated antigens. In some embodiments, the ligand binding domain is bispecific. Antigens have been identified in most of the human cancers, including Burkitt lymphoma, neuroblastoma, melanoma, osteosarcoma, renal cell carcinoma, breast cancer, prostate cancer, lung carcinoma, and colon cancer. TAA's include, without limitation, CD19, CD20, CD22, CD24, CD33, CD38, CD123, CD228, CD138, BCMA, GPC3, CEA, folate receptor (FRα), mesothelin, CD276, gp100, 5T4, GD2, EGFR, MUC-1, PSMA, EpCAM, MCSP, SM5-1, MICA, MICB, ULBP and HER-2. TAAs further include neoantigens, peptide/MHC complexes, and HSP/peptide complexes.

In some embodiments, the antigen binding domain comprises a T-cell receptor or binding fragment thereof that binds to a defined tumor specific peptide-MHC complex. The term “T cell receptor,” or “TCR,” refers to a heterodimeric receptor composed of αβ or γδ chains that pair on the surface of a T cell. Each a, 3, 7, and 6 chain is composed of two Ig-like domains: a variable domain (V) that confers antigen recognition through the complementarity determining regions (CDR), followed by a constant domain (C) that is anchored to cell membrane by a connecting peptide and a transmembrane (TM) region. The TM region associates with the invariant subunits of the CD3 signaling apparatus. Each of the V domains has three CDRs. These CDRs interact with a complex between an antigenic peptide bound to a protein encoded by the major histocompatibility complex (pMHC) (Davis and Bjorkman (1988) Nature, 334, 395-402; Davis et al. (1998) Annu Rev Immunol, 16, 523-544; Murphy (2012), xix, 868 p.).

In some embodiments, the antigen binding domain comprises a natural ligand of a tumor expressed protein or tumor-binding fragment thereof. A non-limiting example is PD1 which binds to PDL1. Another example is the transferrin receptor 1 (TfR1), also known as CD71, a homodimeric protein that is a key regulator of cellular iron homeostasis and proliferation. Although TfR1 is expressed at a low level in a broad variety of cells, it is expressed at higher levels in rapidly proliferating cells, including malignant cells in which overexpression has been associated with poor prognosis. In some embodiments of the invention, the antigen binding domain comprises transferrin or a transferrin receptor-binding fragment thereof.

In some embodiments, the antigen binding domain is specific to a defined tumor associated antigen, such as but not limited to FRα, CEA, 5T4, CA125, SM5-1 or CD71. In some embodiments, the tumor associated antigen can be a tumor-specific peptide-MHC complex. In certain such embodiments, the peptide is a neoantigen. In other embodiments, the tumor associated antigen it a peptide-heat shock protein complex.

In various embodiments, the invention provides a CoStAR which comprises

i. an scFv that binds to carcinoembryonic antigen (CEA), a spacer and transmembrane sequence of CD28, a CD28 signaling domain, and a CD40 signaling domain.

ii. an scFv that binds to CEA, a spacer and transmembrane sequence of CD28, and a CD40 signaling domain.

iii. an scFv that binds to CEA, a spacer and transmembrane sequence of CD28, a CD137 signaling domain, and a CD40 signaling domain.

iv. an scFv that binds to CEA, a spacer and transmembrane sequence of CD28, a CD134 signaling domain, and a CD40 signaling domain.

v. an scFv that binds to CEA, a spacer and transmembrane sequence of CD28, a CD2 signaling domain, and a CD40 signaling domain.

vi. an scFv that binds to CEA, a spacer and transmembrane sequence of CD28, a GITR signaling domain, and a CD40 signaling domain.

vii. an scFv that binds to CEA, a spacer and transmembrane sequence of CD28, a CD29 signaling domain, and a CD40 signaling domain.

viii. an scFv that binds to CEA, a spacer and transmembrane sequence of CD28, a CD150 signaling domain, and a CD40 signaling domain.

ix. an scFv that binds to CEA, a spacer and transmembrane sequence of CD8, a CD28 signaling domain, and a CD40 signaling domain.

x. an scFv that binds to CEA, a spacer and transmembrane sequence of CD8, and a CD40 signaling domain.

xi. an scFv that binds to CEA, a spacer and transmembrane sequence of CD8, a CD137 signaling domain, and a CD40 signaling domain.

xii. an scFv that binds to CEA, a spacer and transmembrane sequence of CD8, a CD134 signaling domain, and a CD40 signaling domain.

xiii. an scFv that binds to CEA, a spacer and transmembrane sequence of CD8, a CD2 signaling domain, and a CD40 signaling domain.

xiv. an scFv that binds to CEA, a spacer and transmembrane sequence of CD8, a GITR signaling domain, and a CD40 signaling domain.

xv. an scFv that binds to CEA, a spacer and transmembrane sequence of CD8, a CD29 signaling domain, and a CD40 signaling domain.

xvi. an scFv that binds to CEA, a spacer and transmembrane sequence of CD8, a CD150 signaling domain, and a CD40 signaling domain.

xvii. an scFv that binds to CEA, a spacer comprising an IgG4 constant region and CD28 transmembrane sequence, a CD28 signaling domain, and a CD40 signaling domain.

xviii. an scFv that binds to CEA, a spacer comprising an IgG4 constant region and CD28 transmembrane sequence, and a CD40 signaling domain.

xix. an scFv that binds to CEA, a spacer comprising an IgG4 constant region and CD28 transmembrane sequence, a CD137 signaling domain, and a CD40 signaling domain.

xx. an scFv that binds to CEA, a spacer comprising an IgG4 constant region and CD28 transmembrane sequence, a CD134 signaling domain, and a CD40 signaling domain.

xxi. an scFv that binds to CEA, a spacer comprising an IgG4 constant region and CD28 transmembrane sequence, a CD2 signaling domain, and a CD40 signaling domain.

xxii. an scFv that binds to CEA, a spacer comprising an IgG4 constant region and CD28 transmembrane sequence, a GITR signaling domain, and a CD40 signaling domain.

xxiii. an scFv that binds to CEA, a spacer comprising an IgG4 constant region and CD28 transmembrane sequence, a CD29 signaling domain, and a CD40 signaling domain.

xxiv. an scFv that binds to CEA, a spacer comprising an IgG4 constant region and CD28 transmembrane sequence, a CD150 signaling domain, and a CD40 signaling domain.

xxv. an scFv that binds to CEA, a spacer comprising an IgG4 constant region and CD28 transmembrane sequence, a first CD40 signaling domain and a second CD40 signaling domain

xxvi. an scFv that binds to CEA, a spacer comprising an IgG4 constant region and CD28 transmembrane sequence, a first CD40 signaling domain and a second mutated CD40 signaling domain

xxvii. a binding domain that binds to PDL1, a short spacer and transmembrane sequence of CD28, a CD28 signaling domain, and a CD40 signaling domain.

xxviii. a binding domain that binds to PDL1, a short spacer and transmembrane sequence of CD28, and a CD40 signaling domain.

xxix. a binding domain that binds to CD155, CD112, or CD113, a CD28 transmembrane domain, a CD28 signaling domain, and a CD40 signaling domain.

xxx. a binding domain that binds to CD155, CD112, or CD113, a CD28 transmembrane domain, and a CD40 signaling domain.

xxxi. an scFv that binds to CEA, a binding domain that binds to PDL1, a short spacer and transmembrane sequence of CD28, a CD28 signaling domain, and a CD40 signaling domain.

xxxii. an scFv that binds to CEA, a binding domain that binds to PDL1, a short spacer and transmembrane sequence of CD28, and a CD40 signaling domain.

xxxiii. an scFv that binds to CEA, a binding domain that binds to CD155, CD112, or CD113, a short spacer and transmembrane sequence of CD28, a CD28 signaling domain, and a CD40 signaling domain.

xxxiv. an scFv that binds to CEA, a binding domain that binds to CD155, CD112, or CD113, a short spacer and transmembrane sequence of CD28, and a CD40 signaling domain.

xxxv. an scFv that binds to CEA, a spacer comprising the CD28 extracellular domain, and transmembrane sequence of CD28, and a NTRK1 signaling domain

xxxvi. an scFv that binds to CEA, a spacer comprising the CD28 extracellular domain and transmembrane sequence of CD28, a NTRK1 signaling domain, and a CD40 signaling domain.

xxxvii. an scFv that binds to CEA, a spacer comprising the CD28 extracellular domain and transmembrane sequence of CD28, a CD28 signaling domain, and a NTRK1 signaling domain.

xxxviii. an scFv that binds to CEA, a spacer comprising the CD28 extracellular domain and transmembrane sequence of CD28, a CD28 signaling domain, a NTRK1 signaling domain, and a CD40 signaling domain.

xxxix. an scFv that binds to CEA, a spacer comprising the ICOS extracellular domain and transmembrane sequence of ICOS, an ICOS signaling domain, and a CD40 signaling domain

xl. an scFv that binds to CEA, a spacer comprising the CD28 extracellular domain and transmembrane sequence of CD28, a CD28 signaling domain, and an ICOS signaling domain.

xli. an scFv that binds to CEA, a spacer comprising the CD28 extracellular domain and transmembrane sequence of CD28, a CD28 signaling domain, an ICOS signaling domain, and a CD40 signaling domain.

xlii. an scFv that binds to CEA, a spacer comprising the CD2 extracellular domain and transmembrane sequence of CD2, a CD2 signaling domain, and a CD40 signaling domain.

xliii. an scFv that binds to CEA, a spacer comprising the CD28 extracellular domain and transmembrane sequence of CD28, a CD28 signaling domain, and a CD2 signaling domain.

xliv. an scFv that binds to CEA, a spacer comprising the CD28 extracellular domain and transmembrane sequence of CD28, a CD28 signaling domain, a CD40 signaling domain, and a CD2 signaling domain.

xlv. an scFv that binds to CEA, a spacer comprising the CD28 extracellular domain and transmembrane sequence of CD28, a CD28 signaling domain, and aCD137 signaling domain.

xlvi. an scFv that binds to CEA, a spacer comprising the CD28 extracellular domain and transmembrane sequence of CD28, a CD28 signaling domain, a CD40 signaling domains, and a CD137 signaling domain.

xlvii. an scFv that binds to CEA, a spacer comprising the CD28 extracellular domain and transmembrane sequence of CD28, a CD28 signaling domain, and a DAP10 signaling domain.

xlviii. an scFv that binds to CEA, a spacer comprising the CD28 extracellular domain and transmembrane sequence of CD28, a CD28 signaling domain, a CD40 signaling domain, and a DAP10 signaling domain.

xlix. an scFv that binds to CEA, a spacer comprising the CD28 extracellular domain and transmembrane sequence of CD28, a CD28 signaling domain, and a CD134 signaling domain.

xlx. an scFv that binds to CEA, a spacer comprising the CD28 extracellular domain and transmembrane sequence of CD28, a CD40 signaling domain, and a CD134 signaling domain.

In some embodiments, the invention provides a CoStAR which comprises an scFv that binds to MSLN linked to the spacer, transmembrane, and signaling domain structure of any one of paragraphs i-xlx.

In some embodiments, the invention provides a CoStAR which comprises an scFv that binds to FolR1linked to the spacer, transmembrane, and signaling domain structure of any one of paragraphs i-xlx.

In some embodiments, the invention provides a CoStAR which comprises the spacer, transmembrane, and signaling domain structure of any one of i-xxxiv and binds to FolR1 by a binding domain which comprises an antigen-binding fragment of scFv MOV19 (SEQ ID NO:9).

In some embodiments, the invention provides a CoStAR which comprises the spacer, transmembrane, and signaling domain structure of any one of i-xxxiv and binds to CEA by a binding domain which comprises an antigen-binding fragment of scFv MFE23 (SEQ ID NO:10).

In some embodiments, the invention provides a CoStAR which comprises the spacer, transmembrane, and signaling domain structure of any one of i-xxvi and xxxi to xxxiv and binds to CEA by a binding domain which comprises an antigen-binding fragment of scFv MFE23(K>Q) (SEQ ID NO:11).

In some embodiments, the invention provides a CoStAR which comprises the spacer, transmembrane, and signaling domain structure of any one of i-xxvi and xxxi to xxxiv and binds to CEA by a binding domain which comprises an antigen-binding fragment of humanized scFv MFE23 (hMFE23) (SEQ ID NO:12).

In some embodiments, the invention provides a CoStAR which comprises the spacer, transmembrane, and signaling domain structure of any one of i-xxvi and xxxi to xxxiv and binds to CEA by a binding domain which comprises an antigen-binding fragment of scFv CEA6 (SEQ ID NO:13).

In some embodiments, the invention provides a CoStAR which comprises the spacer, transmembrane, and signaling domain structure of any one of i-xxvi and xxxi to xxxiv and binds to CEA by a binding domain which comprises an antigen-binding fragment of scFv BW431/26 (SEQ ID NO:14).

In some embodiments, the invention provides a CoStAR which comprises the spacer, transmembrane, and signaling domain structure of any one of i-xxvi and xxxi to xxxiv and binds to CEA by a binding domain which comprises an antigen-binding fragment of scFv HuT84.66(M5A) (SEQ ID NO:15).

In some embodiments, the invention provides a CoStAR which comprises the spacer, transmembrane, and signaling domain structure of any one of i-xxxiv and binds to FolR1 by a binding domain which comprises an antigen-binding fragment of scFv MOV19 (SEQ ID NO:9).

In some embodiments, the invention provides a CoStAR which comprises the spacer, transmembrane, and signaling domain structure of any one of i-xxxiv and xxxi to xxxiv and binds to MSLN by a binding domain which comprises an antigen-binding fragment of scFv SS1 (SEQ ID NO:186).

In some embodiments, the invention provides a CoStAR which comprises the spacer, transmembrane, and signaling domain structure of any one of i-xxvi and xxxi to xxxiv and binds to MSLN by a binding domain which comprises an antigen-binding fragment of scFv M5 (humanized SS1) (SEQ ID NO:187).

In some embodiments, the invention provides a CoStAR which comprises the spacer, transmembrane, and signaling domain structure of any one of i-xxvi and xxxi to xxxiv and binds to MSLN by a binding domain which comprises an antigen-binding fragment of humanized scFv HN1 (SEQ ID NO:188).

In some embodiments, the invention provides a CoStAR which comprises the spacer, transmembrane, and signaling domain structure of any one of i-xxvi and xxxi to xxxiv and binds to MSLN by a binding domain which comprises an antigen-binding fragment of scFv M912 (SEQ ID NO:189).

In some embodiments, the invention provides a CoStAR which comprises the spacer, transmembrane, and signaling domain structure of any one of i-xxvi and xxxi to xxxiv and binds to MSLN by a binding domain which comprises an antigen-binding fragment of scFv HuYP218 (SEQ ID NO:190).

In some embodiments, the invention provides a CoStAR which comprises the spacer, transmembrane, and signaling domain structure of any one of i-xxvi and xxxi to xxxiv and binds to MSLN by a binding domain which comprises an antigen-binding fragment of scFv P4 (SEQ ID NO:191).

In some embodiments, any of the above (including I to xlx) can be used with a CD40, TRAF2, TRAF2/3, or TRAF6 variants provided herein, including those in FIGS. 28, 67, 77A-77C, 83-89D, and 92 (excluding those in FIGS. 78 and 79), and including those in Tables 2, 4-5, and 7-14, and including SEQ ID NOS: 42, 475-479, 494-498, 505, 510-519, and 840-40839, and Consensus sequences A-C.

As use herein, the term “specifically binds” or “is specific for” refers to measurable and reproducible interactions, such as binding between a target and an antibody or antibody moiety that is determinative of the presence of the target in the presence of a heterogeneous population of molecules, including biological molecules. For example, an antibody moiety that specifically binds to a target (which can be an epitope) is an antibody moiety that binds the target with greater affinity, avidity, more readily, and/or with greater duration than its bindings to other targets. In some embodiments, an antibody moiety that specifically binds to an antigen reacts with one or more antigenic determinants of the antigen (for example a cell surface antigen or a peptide/MHC protein complex) with a binding affinity that is at least about 10 times its binding affinity for other targets.

### Spacer

A CoStAR of the invention optionally comprises a spacer region between the antigen binding domain and the costimulatory receptor. As used herein, the term “spacer” refers to the extracellular structural region of a CoStAR that separates the antigen binding domain from the external ligand binding domain of the costimulatory protein. The spacer provides flexibility to access the targeted antigen and receptor ligand. In some embodiments long spacers are employed, for example to target membrane-proximal epitopes or glycosylated antigens (see Guest R. D. et al. The role of extracellular spacer regions in the optimal design of chimeric immune receptors: evaluation of four different scFvs and antigens. J. Immunother. 2005; 28:203-211; Wilkie S. et al., Retargeting of human T cells to tumor-associated MUC1: the evolution of a chimeric antigen receptor. J. Immunol. 2008; 180:4901-4909). In other embodiments, CoStARs bear short spacers, for example to target membrane distal epitopes (see Hudecek M. et al., Receptor affinity and extracellular domain modifications affect tumor recognition by ROR1-specific chimeric antigen receptor T cells. Clin. Cancer Res. 2013; 19:3153-3164; Hudecek M. et al., The nonsignaling extracellular spacer domain of chimeric antigen receptors is decisive for in vivo antitumor activity. Cancer Immunol. Res. 2015; 3:125-135). In some embodiments, the spacer comprises all or part of or is derived from an IgG hinge, including but not limited to IgG1, IgG2, or IgG4. By “derived from an Ig hinge” is meant a spacer comprising insertions, deletions, or mutations in an IgG hinge. In some embodiments, a spacer can comprise all or part of one or more antibody constant domains, such as but not limited to CH2 and/or CH3 domains. In some embodiments, in a spacer comprising all or part of a CH2 domain, the CH2 domain is modified so as not to bind to an Fc receptor. For example, Fc receptor binding in myeloid cells has been found to impair CAR T cell functionality. In some embodiments, the spacer comprises all or part of an Ig-like hinge from CD28, CD8, or other protein comprising a hinge region. In some embodiments of the invention that comprise a spacer, the spacer is from 1 and 50 amino acids in length.

In an non-limiting embodiment, the spacer comprises essentially all of an extracellular domain, for example a CD28 extracellular domain (i.e. from about amino acid 19, 20, 21, or 22 to about amino acid 152) or an extracellular domain of another protein, including but not limited to another TNFR superfamily member. In some embodiments, the spacer comprises a portion of an extracellular domain, for example a portion of a CD28 extracellular domain, and may lack all or most of the Ig domain. In another embodiment, the spacer includes amino acids of CD28 from about 141 to about 152 but not other portions of the CD28 extracellular domain. In another embodiment, the spacer includes amino acids of CD8 from about 128 to about 182 but not other portions of the CD8 extracellular domain.

### Linker

In some embodiments, the CoStAR extracellular domain comprises a linker. Linkers comprise short runs of amino acids used to connect domains, for example a binding domain with a spacer or transmembrane domain. In order for there to be flexibility to bind ligand, a ligand binding domain will usually be connected to a spacer or a transmembrane domain by flexible linker comprising from about 5 to 25 amino acids, such as, for example, AAAGSGGSG (SEQ ID NO:18), GGGGSGGGGSGGGGS (SEQ ID NO:431). In some embodiments, a CoStAR comprises a binding domain joined directly to a transmembrane domain by a linker, and without a spacer. In some embodiments, a CoStAR comprises a binding domain joined directly to a transmembrane by a spacer and without a linker.

### Signaling Domain

As discussed above, in some embodiments, a CoStAR comprises a full length primary costimulatory receptor which can comprise an extracellular ligand binding and intracellular signaling portion of, without limitation, CD2, CD9, CD26, CD27, CD28, CD29, CD38, CD40, CD43, CD46, CD49d, CD55, CD73, CD81, CD82, CD99, CD100, CD134 (OX40), CD137 (41BB), CD150 (SLAM), CD270 (HVEM), CD278 (ICOS), CD357 (GITR), or EphB6. In other embodiments, the costimulatory receptor comprises a chimeric protein, for instance comprising an extracellular ligand binding domain of one of the aforementioned proteins and an intracellular signaling domain of another of the aforementioned proteins. In some embodiments, the signaling portion of the CoStAR comprises a single signaling domain. In other embodiments, the signaling portion of the CoStAR comprises a second intracellular signaling domain such as but not limited to: CD2, CD27, CD28, CD40, CD134 (OX40), CD137 (4-1BB), CD150 (SLAM). In some embodiments, the first and second intracellular signaling domains are the same. In other embodiments, the first and second intracellular signaling domains are different. In some embodiments, the costimulatory receptor is capable of dimerization. Without being bound by theory, it is thought that CoStARs dimerize or associate with other accessory molecules for signal initiation. In some embodiments, CoStARs dimerize or associate with accessory molecules through transmembrane domain interactions. In some embodiments, dimerization or association with accessory molecules is assisted by costimulatory receptor interactions in the intracellular portion, and/or the extracellular portion of the costimulatory receptor.

### Transmembrane Domain

Although the main function of the transmembrane is to anchor the CoStAR in the T cell membrane, in some embodiments, the transmembrane domain influences CoStAR function. In some embodiments, the transmembrane domain is comprised by the full length primary costimulatory receptor domain. In embodiments of the invention wherein the CoStAR construct comprises an extracellular domain of one receptor and an intracellular signaling domain of a second receptor, the transmembrane domain can be that of the extracellular domain or the intracellular domain. In some embodiments, the transmembrane domain is from CD4, CD8a, CD28, or ICOS. Gueden et al. associated use of the ICOS transmembrane domain with increased CAR T cell persistence and overall anti-tumor efficacy (Guedan S. et al., Enhancing CAR T cell persistence through ICOS and 4-1BB costimulation. JCI Insight. 2018; 3:96976). In some embodiments, the transmembrane domain comprises a hydrophobic a helix that spans the cell membrane.

In some embodiments, the transmembrane domain comprises amino acids of the CD28 transmembrane domain from about amino acid 153 to about amino acid 179. In another embodiment, the transmembrane domain comprises amino acids of the CD8 transmembrane domain from about amino acid 183 to about amino acid 203. In some embodiments, the CoStARs of the invention may include several amino acids between the transmembrane domain and signaling domain. For example, in one construct described herein the link from a CD8 transmembrane domain to a signaling domain comprises several amino acids of the CD8 cytoplasmic domain (e.g., amino acids 204-210 of CD8).

### Variants

In some embodiments, amino acid sequence variants of the antibody moieties or other moieties provided herein are contemplated. For example, it may be desirable to improve the binding affinity and/or other biological properties of the antibody moiety. Amino acid sequence variants of an antibody moiety may be prepared by introducing appropriate modifications into the nucleotide sequence encoding the antibody moiety, or by peptide synthesis. Such modifications include, for example, deletions from, and/or insertions into and/or substitutions of residues within the amino acid sequences of the antibody moiety. Any combination of deletion, insertion, and substitution can be made to arrive at the final construct, provided that the final construct possesses the desired characteristics, e.g., antigen-binding.

In some embodiments, antibody binding domain moieties comprising one or more amino acid substitutions, deletions, or insertions are provided. Sites of interest for mutational changes include the antibody binding domain heavy and light chain variable regions (VRs) and frameworks (FRs). Amino acid substitutions may be introduced into a binding domain of interest and the products screened for a desired activity, e.g., retained/improved antigen binding or decreased immunogenicity. In some embodiments, amino acid substitutions may be introduced into one or more of the primary co-stimulatory receptor domain (extracellular or intracellular), secondary costimulatory receptor domain, or extracellular co-receptor domain. Accordingly, the invention encompasses CoStAR proteins and component parts particularly disclosed herein as well as variants thereof, i.e. CoStAR proteins and component parts having at least 75%, at least 80%, at least 85%, at least 87%, at least 90%, at least 91%, at least 92%, at least 93%, at least 94%, at least 95%, at least 96%, at least 97%, at least 98%, at least 99% sequence identity to the amino acid sequences particularly disclosed herein. The terms “percent similarity,” “percent identity,” and “percent homology” when referring to a particular sequence are used as set forth in the University of Wisconsin GCG software program BestFit. Other algorithms may be used, e.g. BLAST, psiBLAST or TBLASTN (which use the method of Altschul et al. (1990) J. Mol. Biol. 215: 405-410), FASTA (which uses the method of Pearson and Lipman (1988) PNAS USA 85: 2444-2448).

Particular amino acid sequence variants may differ from a reference sequence by insertion, addition, substitution or deletion of 1 amino acid, 2, 3, 4, 5-10, 10-20 or 20-30 amino acids. In some embodiments, a variant sequence may comprise the reference sequence with 1, 2, 3, 4, 5, 6, 7, 8, 9, 10 or more residues inserted, deleted or substituted. For example, 5, 10, 15, up to 20, up to 30 or up to 40 residues may be inserted, deleted or substituted.

In some preferred embodiments, a variant may differ from a reference sequence by 1, 2, 3, 4, 5, 6, 7, 8, 9, 10 or more conservative substitutions. Conservative substitutions involve the replacement of an amino acid with a different amino acid having similar properties. For example, an aliphatic residue may be replaced by another aliphatic residue, a non-polar residue may be replaced by another non-polar residue, an acidic residue may be replaced by another acidic residue, a basic residue may be replaced by another basic residue, a polar residue may be replaced by another polar residue or an aromatic residue may be replaced by another aromatic residue. Conservative substitutions may, for example, be between amino acids within the following groups:

Conservative substitutions are shown in Table 6 below.

TABLE 6
Amino Acid Substitutions

| Original | | Preferred |
|---|---|---|
| Residue | Exemplary Substitutions | Substitutions |
| Ala (A) | Val; Leu; Ile | Val |
| Arg (R) | Lys; Gln; Asn | Lys |
| Asn (N) | Gln; His; Asp; Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped into different classes according to common side-chain properties: a. hydrophobic: Norleucine, Met, Ala, Val, Leu, Ile; b. neutral hydrophilic: Cys, Ser, Thr, Asn, Gln; c. acidic: Asp, Glu; d. basic: His, Lys, Arg; e. residues that influence chain orientation: Gly, Pro; aromatic: Trp, Tyr, Phe. Non-conservative substitutions will entail exchanging a member of one of these classes for another class.

### Cells

The cells used in the present invention may be any lymphocyte that is useful in adoptive cell therapy, such as a T-cell or a natural killer (NK) cell, an NKT cell, a gamma/delta T-cell or T regulatory cell. The cells may be allogeneic or autologous to the patient.

T cells or T lymphocytes are a type of lymphocyte that have a central role in cell-mediated immunity. They can be distinguished from other lymphocytes, such as B cells and natural killer cells (NK cells), by the presence of a T-cell receptor (TCR) on the cell surface. There are various types of T cell, as summarized below. Cytotoxic T cells (TC cells, or CTLs) destroy virally infected cells and tumor cells, and are also implicated in transplant rejection. CTLs express the CD8 molecule at their surface.

These cells recognize their targets by binding to antigen associated with MHC class I, which is present on the surface of all nucleated cells. Through IL-10, adenosine and other molecules secreted by regulatory T cells, the CD8+ cells can be inactivated to an anergic state, which prevent autoimmune diseases such as experimental autoimmune encephalomyelitis.

Memory T cells are a subset of antigen-specific T cells that persist long-term after an infection has resolved. They quickly expand to large numbers of effector T cells upon re-exposure to their cognate antigen, thus providing the immune system with “memory” against past infections. Memory T cells comprise three subtypes: central memory T cells (TCM cells) and two types of effector memory T cells (TEM cells and TEMRA cells). Memory cells may be either CD4+ or CD8+. Memory T cells typically express the cell surface protein CD45RO. Regulatory T cells (Treg cells), formerly known as suppressor T cells, are crucial for the maintenance of immunological tolerance. Their major role is to shut down T cell-mediated immunity toward the end of an immune reaction and to suppress auto-reactive T cells that escaped the process of negative selection in the thymus.

Two major classes of CD4+ Treg cells have been described naturally occurring Treg cells and adaptive Treg cells. Naturally occurring Treg cells (also known as CD4⁺CD25⁺FoxP3⁺ Treg cells) arise in the thymus and have been linked to interactions between developing T cells with both myeloid (CD11c⁺) and plasmacytoid (CD123⁺) dendritic cells that have been activated with TSLP. Naturally occurring Treg cells can be distinguished from other T cells by the presence of an intracellular molecule called FoxP3. Adaptive Treg cells (also known as Tr1 cells or Th3 cells) may originate during a normal immune response.

Natural Killer Cells (or NK cells) are a type of cytolytic cell which form part of the innate immune system. NK cells provide rapid responses to innate signals from virally infected cells in an MHC independent manner. NK cells (belonging to the group of innate lymphoid cells) are defined as large granular lymphocytes (LGL) and constitute the third kind of cells differentiated from the common lymphoid progenitor generating B and T lymphocytes.

In some embodiments, therapeutic cells of the invention comprise autologous cells engineered to express a CoStAR. In some embodiments, therapeutic cells of the invention comprise allogeneic cells engineered to express a CoStAR. Autologous cells expressing CoStARs may be advantageous in avoiding graft-versus-host disease (GVHD) due to TCR-mediated recognition of recipient alloantigens. Also, the immune system of a CoStAR recipient could attack the infused CoStAR cells, causing rejection. In certain embodiments, to prevent GVHD, and to reduce rejection, endogenous TcR is removed from allogeneic CoStAR cells by genome editing.

### Nucleic Acids

An aspect of the invention provides a nucleic acid sequence of the invention, encoding any of the CoStARs, polypeptides, or proteins described herein (including functional portions and functional variants thereof). As used herein, the terms “polynucleotide”, “nucleotide”, and “nucleic acid” are intended to be synonymous with each other. It will be understood by a skilled person that numerous different polynucleotides and nucleic acids can encode the same polypeptide as a result of the degeneracy of the genetic code. In addition, it is to be understood that skilled persons may, using routine techniques, make nucleotide substitutions that do not affect the polypeptide sequence encoded by the polynucleotides described here to reflect the codon usage of any particular host organism in which the polypeptides are to be expressed, e.g. codon optimization. Nucleic acids according to the invention may comprise DNA or RNA. They may be single stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of the present invention, it is to be understood that the polynucleotides may be modified by any method available in the art. Such modifications may be carried out in order to enhance the in vivo activity or life span of polynucleotides of interest.

The terms “variant”, “homologue” or “derivative” in relation to a nucleotide sequence include any substitution of, variation of, modification of, replacement of, deletion of or addition of one (or more) nucleic acid from or to the sequence.

The nucleic acid sequence may encode the CoStAR proteins including without limitation any one of SEQ ID NOS:42-247 or a variant thereof. The nucleotide sequence may comprise a codon optimized nucleic acid sequence shown engineered for expression in human cells.

The invention also provides a nucleic acid sequence which comprises a nucleic acid sequence encoding a CoStAR and a further nucleic acid sequence encoding a T-cell receptor (TCR) and/or chimeric antigen receptor (CAR).

The nucleic acid sequences may be joined by a sequence allowing co-expression of the two or more nucleic acid sequences. For example, the construct may comprise an internal promoter, an internal ribosome entry sequence (IRES) sequence or a sequence encoding a cleavage site. The cleavage site may be self-cleaving, such that when the polypeptide is produced, it is immediately cleaved into the discrete proteins without the need for any external cleavage activity. Various self-cleaving sites are known, including the Foot- and Mouth disease virus (FMDV) and the 2A self-cleaving peptide. The co-expressing sequence may be an internal ribosome entry sequence (IRES). The co-expressing sequence may be an internal promoter.

### Vectors

In an aspect, the present invention provides a vector which comprises a nucleic acid sequence or nucleic acid construct of the invention.

Such a vector may be used to introduce the nucleic acid sequence(s) or nucleic acid construct(s) into a host cell so that it expresses one or more CoStAR(s) according to the first aspect of the invention and, optionally, one or more other proteins of interest (POI), for example a TCR or a CAR. The vector may, for example, be a plasmid or a viral vector, such as a retroviral vector or a lentiviral vector, or a transposon-based vector or synthetic mRNA.

The nucleic acids of the present invention may also be used for nucleic acid immunization and gene therapy, using standard gene delivery protocols. Methods for gene delivery are known in the art. See, e.g., U.S. Pat. Nos. 5,399,346, 5,580,859, 5,589,466, incorporated by reference herein in their entireties.

Vectors derived from retroviruses, such as the lentivirus, are suitable tools to achieve long-term gene transfer since they allow long-term, stable integration of a transgene or transgenes and its propagation in daughter cells. The vector may be capable of transfecting or transducing a lymphocyte including a T cell or an NK cell. The present invention also provides vectors in which a nucleic acid of the present invention is inserted. The expression of natural or synthetic nucleic acids encoding a CoStAR, and optionally a TCR or CAR is typically achieved by operably linking a nucleic acid encoding the CoStAR and TCR/CAR polypeptide or portions thereof to one or more promoters, and incorporating the construct into an expression vector.

Additional promoter elements, e.g., enhancers, regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the thymidine kinase (tk) promoter, the spacing between promoter elements can be increased to 50 bp apart before activity begins to decline.

One example of a suitable promoter is the immediate early cytomegalovirus (CMV) promoter sequence. This promoter sequence is a strong constitutive promoter sequence capable of driving high levels of expression of any polynucleotide sequence operatively linked thereto. Another example of a suitable promoter is Elongation Growth Factor-1α (EF-1α). However, other constitutive promoter sequences may also be used, including, but not limited to the simian virus 40 (SV40) early promoter, mouse mammary tumor virus (MMTV), human immunodeficiency virus (HIV) long terminal repeat (LTR) promoter, MoMuLV promoter, MSCV promoter, MND promoter, an avian leukemia virus promoter, an Epstein-Barr virus immediate early promoter, a Rous sarcoma virus promoter, as well as human gene promoters such as, but not limited to, the actin promoter, the myosin promoter, the hemoglobin promoter, and the creatine kinase promoter.

The vectors can be suitable for replication and integration in eukaryotic cells. Typical cloning vectors contain transcription and translation terminators, initiation sequences, and promoters useful for regulation of the expression of the desired nucleic acid sequence. Viral vector technology is well known in the art and is described, for example, in Sambrook et al. (2001, Molecular Cloning: A Laboratory Manual, Cold Spring Harbor Laboratory, New York), and in other virology and molecular biology manuals, see also, WO 01/96584; WO 01/29058; and U.S. Pat. No. 6,326,193). In some embodiments, the constructs expressed are as shown in SEQ ID NOS:42-247. In some embodiments the nucleic acids are multi-cistronic constructs that permit the expression of multiple transgenes (e.g., CoStAR and a TCR and/or CAR etc.) under the control of a single promoter. In some embodiments, the transgenes (e.g., CoStAR and a TCR and/or CAR etc.) are separated by a self-cleaving 2A peptide. Examples of 2A peptides useful in the nucleic acid constructs of the invention include F2A, P2A, T2A and E2A. In other embodiments of the invention, the nucleic acid construct of the invention is a multi-cistronic construct comprising two promoters; one promoter driving the expression of CoStAR and the other promoter driving the expression of the TCR or CAR. In some embodiments, the dual promoter constructs of the invention are uni-directional. In other embodiments, the dual promoter constructs of the invention are bi-directional. In order to assess the expression of the CoStAR polypeptide or portions thereof, the expression vector to be introduced into a cell can also contain either a selectable marker gene or a reporter gene or both to facilitate identification and selection of expressing cells from the population of cells sought to be transfected or transduced through viral vectors.

### Sources of Cells

Prior to expansion and genetic modification, a source of cells (e.g., immune effector cells, e.g., T cells or NK cells) is obtained from a subject. The term “subject” is intended to include living organisms in which an immune response can be elicited (e.g., mammals). Examples of subjects include humans, dogs, cats, mice, rats, and transgenic species thereof. T cells can be obtained from a number of sources, including peripheral blood mononuclear cells, bone marrow, lymph node tissue, cord blood, thymus tissue, tissue from a site of infection, ascites, pleural effusion, spleen tissue, and tumors.

In one aspect, T cells are isolated from peripheral blood lymphocytes by lysing the red blood cells and depleting the monocytes, for example, by centrifugation through a PERCOLL™ gradient or by counterflow centrifugal elutriation. T cell may be collected at an apheresis center and cell storage facility where T cells can be harvested, maintained, and easily transferred. The T cells can be cryopreserved and stored for later use. An acceptable duration of storage may be determined and validated and can be up to 6 months, up to a year, or longer.

In some embodiments, Tumor infiltrating cells (TILs) are isolated and/or expanded from a tumor, for example by a fragmented, dissected, or enzyme digested tumor biopsy or mass. The TILs may be produced in a two-stage process using a tumor biopsy as the starting material: Stage 1 (generally performed over 2-3 hours) initial collection and processing of tumor material using dissection, enzymatic digestion and homogenization to produce a single cell suspension which can be directly cryopreserved to stabilize the starting material for subsequent manufacture and Stage 2 which can occur days or years later. Stage 2 may be performed over 4 weeks, which may be a continuous process starting with thawing of the product of Stage 1 and growth of the TIL out of the tumor starting material (about 2 weeks) followed by a rapid expansion process of the TIL cells (about 2 weeks) to increase the amount of cells and therefore dose. The TILs maybe concentrated and washed prior to formulation as a liquid suspension of cells.

The TIL population can be transduced at any point following collection. In some embodiments, a cryopreserved TIL population is transduced to express a CoStAR following thawing. In some embodiments, a TIL population is transduced to express a CoStAR during outgrowth or initial expansion from tumor starting material. In some embodiments, a TIL population is transduced to express a CoStAR during REP, for example but not limited to from about day 8 to about day 10 of REP. An exemplary TIL preparation is described in Applicant's U.S. patent application Ser. No. 62/951,559, filed Dec. 20, 2019.

A specific subpopulation of T cells, such as CD3+, CD28+, CD4+, CD8+, CD45RA+, and CD45RO+ T cells, can be further isolated by positive or negative selection techniques. For example, in one aspect, T cells are isolated by incubation with anti-CD3/anti-CD28-conjugated beads, such as DYNABEADS® M-450 CD3/CD28 T, for a time period sufficient for positive selection of the desired T cells. In one aspect, the time period is about 30 minutes. In a further aspect, the time period ranges from 30 minutes to 36 hours or longer and all integer values there between. In a further aspect, the time period is at least 1, 2, 3, 4, 5, or 6 hours. In yet another preferred aspect, the time period is 10 to 24 hours. In one aspect, the incubation time period is 24 hours. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells. Thus, by simply shortening or lengthening the time T cells are allowed to bind to the CD3/CD28 beads and/or by increasing or decreasing the ratio of beads to T cells (as described further herein), subpopulations of T cells can be preferentially selected for or against at culture initiation or at other time points during the process. Additionally, by increasing or decreasing the ratio of anti-CD3 and/or anti-CD28 antibodies on the beads or other surface, subpopulations of T cells can be preferentially selected for or against at culture initiation or at other desired time points. The skilled artisan would recognize that multiple rounds of selection can also be used in the context of this invention. In certain aspects, it may be desirable to perform the selection procedure and use the “unselected” cells in the activation and expansion process. “Unselected” cells can also be subjected to further rounds of selection.

Enrichment of a T cell population by negative selection can be accomplished with a combination of antibodies directed to surface markers unique to the negatively selected cells. One method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD16, HLA-DR, and CD8. In certain aspects, it may be desirable to enrich for or positively select for regulatory T cells which typically express CD4+, CD25+, CD62Lhi, GITR+, CD137, PD1, TIM3, LAG-3, CD150 and FoxP3+. Alternatively, in certain aspects, T regulatory cells are depleted by anti-CD25 conjugated beads or other similar method of selection.

The methods described herein can include, e.g., selection of a specific subpopulation of immune effector cells, e.g., T cells, that are a T regulatory cell-depleted population, CD25+ depleted cells, using, e.g., a negative selection technique, e.g., described herein. Preferably, the population of T regulatory depleted cells contains less than 30%, 25%, 20%, 15%, 10%, 5%, 4%, 3%, 2%, 1% of CD25+ cells.

A specific subpopulation of CoStAR effector cells that specifically bind to a target antigen can be enriched for by positive selection techniques. For example, in some embodiments, effector cells are enriched for by incubation with target antigen-conjugated beads for a time period sufficient for positive selection of the desired abTCR effector cells. In some embodiments, the time period is about 30 minutes. In some embodiments, the time period ranges from 30 minutes to 36 hours or longer (including all ranges between these values). In some embodiments, the time period is at least one, 2, 3, 4, 5, or 6 hours. In some embodiments, the time period is 10 to 24 hours. In some embodiments, the incubation time period is 24 hours. For isolation of effector cells present at low levels in the heterogeneous cell population, use of longer incubation times, such as 24 hours, can increase cell yield. Longer incubation times may be used to isolate effector cells in any situation where there are few effector cells as compared to other cell types. The skilled artisan would recognize that multiple rounds of selection can also be used in the context of this invention.

T cells for stimulation can also be frozen after a washing step. After the washing step that removes plasma and platelets, the cells may be suspended in a freezing solution. While many freezing solutions and parameters are known in the art and will be useful in this context, one method involves using PBS containing 20% DMSO and 8% human serum albumin, or culture media containing 10% Dextran 40 and 5% Dextrose, 20% Human Serum Albumin and 7.5% DMSO, or 31.25% Plasmalyte-A, 31.25% Dextrose 5%, 0.45% NaCl, 10% Dextran 40 and 5% Dextrose, 20% Human Serum Albumin, and 7.5% DMSO or other suitable cell freezing media containing for example, Hespan and PlasmaLyte A, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

### Allogneic CoStAR

In embodiments described herein, the immune effector cell can be an allogeneic immune effector cell, e.g., T cell or NK cell. For example, the cell can be an allogeneic T cell, e.g., an allogeneic T cell lacking expression of endogenous T cell receptor (TCR) and/or human leukocyte antigen (HLA), e.g., HLA class I and/or HLA class II.

A T cell lacking a functional endogenous TCR can be, e.g., engineered such that it does not express any functional TCR on its surface, engineered such that it does not express one or more subunits that comprise a functional TCR (e.g., engineered such that it does not express (or exhibits reduced expression) of TCR alpha, TCR beta, TCR gamma, TCR delta, TCR epsilon, and/or TCR zeta) or engineered such that it produces very little functional TCR on its surface. Alternatively, the T cell can express a substantially impaired TCR, e.g., by expression of mutated or truncated forms of one or more of the subunits of the TCR. The term “substantially impaired TCR” means that this TCR will not elicit an adverse immune reaction in a host.

A T cell described herein can be, e.g., engineered such that it does not express a functional HLA on its surface. For example, a T cell described herein, can be engineered such that cell surface expression HLA, e.g., HLA class 1 and/or HLA class II, is downregulated. In some aspects, downregulation of HLA may be accomplished by reducing or eliminating expression of beta-2 microglobulin (B2M).

In some embodiments, the T cell can lack a functional TCR and a functional HLA, e.g., HLA class I and/or HLA class II. Modified T cells that lack expression of a functional TCR and/or HLA can be obtained by any suitable means, including a knock out or knock down of one or more subunit of TCR or HLA. For example, the T cell can include a knock down of TCR and/or HLA using siRNA, shRNA, clustered regularly interspaced short palindromic repeats (CRISPR) transcription-activator like effector nuclease (TALEN), or zinc finger endonuclease (ZFN).

In some embodiments, the allogeneic cell can be a cell which does not expresses or expresses at low levels an inhibitory molecule, e.g. a cell engineered by any method described herein. For example, the cell can be a cell that does not express or expresses at low levels an inhibitory molecule, e.g., that can decrease the ability of a CoStAR-expressing cell to mount an immune effector response. Examples of inhibitory molecules include PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, Gal9, adenosine, and TGFR beta. Inhibition of an inhibitory molecule, e.g., by inhibition at the DNA, RNA or protein level, can optimize a CAR-expressing cell performance. In embodiments, an inhibitory nucleic acid, e.g., an inhibitory nucleic acid, e.g., a dsRNA, e.g., an siRNA or shRNA, a clustered regularly interspaced short palindromic repeats (CRISPR), a transcription-activator like effector nuclease (TALEN), or a zinc finger endonuclease (ZFN), e.g., as described herein, can be used.

Use of siRNA or shRNA to Inhibit Endogenous TCR or HLA

In some embodiments, TCR expression and/or HLA expression can be inhibited using siRNA or shRNA that targets a nucleic acid encoding a TCR and/or HLA, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, Gal9, adenosine, and TGFR beta), in a T cell.

Expression of siRNA and shRNAs in T cells can be achieved using any conventional expression system, e.g., such as a lentiviral expression system. Exemplary shRNAs that downregulate expression of components of the TCR are described, e.g., in US Publication No.: 2012/0321667. Exemplary siRNA and shRNA that downregulate expression of HLA class I and/or HLA class II genes are described, e.g., in U.S. publication No.: US 2007/0036773.

CRISPR to Inhibit TCR or HLA

“CRISPR” or “CRISPR to inhibit TCR and/or HLA” as used herein refers to a set of clustered regularly interspaced short palindromic repeats, or a system comprising such a set of repeats. “Cas”, as used herein, refers to a CRISPR-associated protein. A “CRISPR/Cas” system refers to a system derived from CRISPR and Cas which can be used to silence or mutate a TCR and/or HLA gene, and/or an inhibitory molecule described herein (e.g., PD1, PD-L1, PD-L2, CTLA4, TIM3, CEACAM (e.g., CEACAM-1, CEACAM-3 and/or CEACAM-5), LAG3, VISTA, BTLA, TIGIT, LAIR1, CD160, 2B4, CD80, CD86, B7-H3 (CD276), B7-H4 (VTCN1), HVEM (TNFRSF14 or CD270), KIR, A2aR, MHC class I, MHC class II, GAL9, adenosine, and TGFR beta).

Naturally-occurring CRISPR/Cas systems are found in approximately 40% of sequenced eubacteria genomes and 90% of sequenced archaea. Grissa et al. (2007) BMC Bioinformatics 8: 172. This system is a type of prokaryotic immune system that confers resistance to foreign genetic elements such as plasmids and phages and provides a form of acquired immunity. Barrangou et al. (2007) Science 315: 1709-1712; Marragini et al. (2008) Science 322: 1843-1845.

Activation and Expansion of T Cells

T cells may be activated and expanded generally using methods as described, for example, in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005.

Generally, the T cells of the invention may be expanded by contact with a surface having attached thereto an agent that stimulates a CD3/TCR complex associated signal and a ligand that stimulates a costimulatory molecule on the surface of the T cells. In particular, T cell populations may be stimulated as described herein, such as by contact with an anti-CD3 antibody, or antigen-binding fragment thereof, or an anti-CD2 antibody immobilized on a surface, or by contact with a protein kinase C activator (e.g., bryostatin) in conjunction with a calcium ionophore. For co-stimulation of an accessory molecule on the surface of the T cells, a ligand that binds the accessory molecule is used. For example, a population of T cells can be contacted with an anti-CD3 antibody and an anti-CD28 antibody, under conditions appropriate for stimulating proliferation of the T cells. To stimulate proliferation of either CD4+ T cells or CD8+ T cells, an anti-CD3 antibody and an anti-CD28 antibody can be used. Examples of an anti-CD28 antibody include 9.3, B-T3, XR-CD28 (Diaclone, Besancon, France) can be used as can other methods commonly known in the art (Berg et al., Transplant Proc. 30(8):3975-3977, 1998; Haanen et al., J. Exp. Med. 190(9):13191328, 1999; Garland et al., J. Immunol Meth. 227(1-2):53-63, 1999).

In some embodiments, expansion can be performed using flasks or containers, or gas-permeable containers known by those of skill in the art and can proceed for 7 days, 8 days, 9 days, 10 days, 11 days, 12 days, 13 days, or 14 days, about 7 days to about 14 days, about 8 days to about 14 days, about 9 days to about 14 days, about 10 days to about 14 days, about 11 days to about 14 days, about 12 days to about 14 days, or about 13 days to about 14 days. In some embodiments, the second TIL expansion can proceed for about 14 days.

In some embodiments, the expansion can be performed using non-specific T-cell receptor stimulation in the presence of interleukin-2 (IL-2) or interleukin-15 (IL-15). The non-specific T-cell receptor stimulus can include, for example, an anti-CD3 antibody, such as about 30 ng/ml of OKT3, a mouse monoclonal anti-CD3 antibody (commercially available from Ortho-McNeil, Raritan, N.J. or Miltenyi Biotech, Auburn, Calif.) or UHCT-1 (commercially available from BioLegend, San Diego, Calif., USA). CoStAR cells can be expanded in vitro by including one or more antigens, including antigenic portions thereof, such as epitope(s), of a cancer, which can be optionally expressed from a vector, such as a human leukocyte antigen A2 (HLA-A2) binding peptide, e.g., 0.3 uM MART-1:26-35 (27L) or gp100:209-217 (210M), optionally in the presence of a T-cell growth factor, such as 300 IU/mL IL-2 or IL-15. Other suitable antigens may include, e.g., NY-ESO-1, TRP-1, TRP-2, tyrosinase cancer antigen, MAGE-A3, SSX-2, and VEGFR2, or antigenic portions thereof. CoStAR cells may also be rapidly expanded by re-stimulation with the same antigen(s) of the cancer pulsed onto HLA-A2-expressing antigen-presenting cells. Alternatively, the CoStAR cells can be further stimulated with, e.g., example, irradiated, autologous lymphocytes or with irradiated HLA-A2+ allogeneic lymphocytes and IL-2. In some embodiments, the stimulation occurs as part of the expansion. In some embodiments, the expansion occurs in the presence of irradiated, autologous lymphocytes or with irradiated HLA-A2+ allogeneic lymphocytes and IL-2.

In some embodiments, the cell culture medium comprises IL-2. In some embodiments, the cell culture medium comprises about 1000 IU/mL, about 1500 IU/mL, about 2000 IU/mL, about 2500 IU/mL, about 3000 IU/mL, about 3500 IU/mL, about 4000 IU/mL, about 4500 IU/mL, about 5000 IU/mL, about 5500 IU/mL, about 6000 IU/mL, about 6500 IU/mL, about 7000 IU/mL, about 7500 IU/mL, or about 8000 IU/mL, or between 1000 and 2000 IU/mL, between 2000 and 3000 IU/mL, between 3000 and 4000 IU/mL, between 4000 and 5000 IU/mL, between 5000 and 6000 IU/mL, between 6000 and 7000 IU/mL, between 7000 and 8000 IU/mL, or between 8000 IU/mL of IL-2.

In some embodiments, the cell culture medium comprises OKT3 antibody. In some embodiments, the cell culture medium comprises about 0.1 ng/mL, about 0.5 ng/mL, about 1 ng/mL, about 2.5 ng/mL, about 5 ng/mL, about 7.5 ng/mL, about 10 ng/mL, about 15 ng/mL, about 20 ng/mL, about 25 ng/mL, about 30 ng/mL, about 35 ng/mL, about 40 ng/mL, about 50 ng/mL, about 60 ng/mL, about 70 ng/mL, about 80 ng/mL, about 90 ng/mL, about 100 ng/mL, about 200 ng/mL, about 500 ng/mL, about 1 μg/mL or between 0.1 ng/mL and 1 ng/mL, between 1 ng/mL and 5 ng/mL, between 5 ng/mL and 10 ng/mL, between 10 ng/mL and 20 ng/mL, between 20 ng/mL and 30 ng/mL, between 30 ng/mL and 40 ng/mL, between 40 ng/mL and 50 ng/mL, or between 50 ng/mL and 100 ng/mL of OKT3 antibody.

In some embodiments, a combination of IL-2, IL-7, IL-15, and/or IL-21 are employed as a combination during the expansion. In some embodiments, IL-2, IL-7, IL-15, and/or IL-21 as well as any combinations thereof can be included during the expansion. In some embodiments, a combination of IL-2, IL-15, and IL-21 are employed as a combination during the expansion. In some embodiments, IL-2, IL-15, and IL-21 as well as any combinations thereof can be included.

In some embodiments, the expansion can be conducted in a supplemented cell culture medium comprising IL-2, OKT-3, and antigen-presenting feeder cells.

In some embodiments, the expansion culture media comprises about 500 IU/mL of IL-15, about 400 IU/mL of IL-15, about 300 IU/mL of IL-15, about 200 IU/mL of IL-15, about 180 IU/mL of IL-15, about 160 IU/mL of IL-15, about 140 IU/mL of IL-15, about 120 IU/mL of IL-15, or about 100 IU/mL of IL-15, or about 500 IU/mL of IL-15 to about 100 IU/mL of IL-15, or about 400 IU/mL of IL-15 to about 100 IU/mL of IL-15 or about 300 IU/mL of IL-15 to about 100 IU/mL of IL-15 or about 200 IU/mL of IL-15, or about 180 IU/mL of IL-15.

In some embodiments, the expansion culture media comprises about 20 IU/mL of IL-21, about 15 IU/mL of IL-21, about 12 IU/mL of IL-21, about 10 IU/mL of IL-21, about 5 IU/mL of IL-21, about 4 IU/mL of IL-21, about 3 IU/mL of IL-21, about 2 IU/mL of IL-21, about 1 IU/mL of IL-21, or about 0.5 IU/mL of IL-21, or about 20 IU/mL of IL-21 to about 0.5 IU/mL of IL-21, or about 15 IU/mL of IL-21 to about 0.5 IU/mL of IL-21, or about 12 IU/mL of IL-21 to about 0.5 IU/mL of IL-21, or about 10 IU/mL of IL-21 to about 0.5 IU/mL of IL-21, or about 5 IU/mL of IL-21 to about 1 IU/mL of IL-21, or about 2 IU/mL of IL-21. In some embodiments, the cell culture medium comprises about 1 IU/mL of IL-21, or about 0.5 IU/mL of IL-21.

In some embodiments the antigen-presenting feeder cells (APCs) are PBMCs. In some embodiments, the ratio of CoStAR cells to PBMCs and/or antigen-presenting cells in the expansion is about 1 to 25, about 1 to 50, about 1 to 100, about 1 to 125, about 1 to 150, about 1 to 175, about 1 to 200, about 1 to 225, about 1 to 250, about 1 to 275, about 1 to 300, about 1 to 325, about 1 to 350, about 1 to 375, about 1 to 400, or about 1 to 500, or between 1 to 50 and 1 to 300, or between 1 to 100 and 1 to 200.

In certain aspects, the primary stimulatory signal and the costimulatory signal for the T cell may be provided by different protocols. For example, the agents providing each signal may be in solution or coupled to a surface. When coupled to a surface, the agents may be coupled to the same surface (i.e., in “cis” formation) or to separate surfaces (i.e., in “trans” formation). Alternatively, one agent may be coupled to a surface and the other agent in solution. In one aspect, the agent providing the costimulatory signal is bound to a cell surface and the agent providing the primary activation signal is in solution or coupled to a surface. In certain aspects, both agents can be in solution. In one aspect, the agents may be in soluble form, and then cross-linked to a surface, such as a cell expressing Fc receptors or an antibody or other binding agent which will bind to the agents. In this regard, see for example, U.S. Patent Application Publication Nos. 20040101519 and 20060034810 for artificial antigen presenting cells (aAPCs) that are contemplated for use in activating and expanding T cells in the present invention.

In one aspect, the two agents are immobilized on beads, either on the same bead, i.e., “cis,” or to separate beads, i.e., “trans.” By way of example, the agent providing the primary activation signal is an anti-CD3 antibody or an antigen-binding fragment thereof and the agent providing the costimulatory signal is an anti-CD28 antibody or antigen-binding fragment thereof; and both agents are co-immobilized to the same bead in equivalent molecular amounts. In one aspect, a 1:1 ratio of each antibody bound to the beads for CD4+ T cell expansion and T cell growth is used. In certain aspects of the present invention, a ratio of anti CD3:CD28 antibodies bound to the beads is used such that an increase in T cell expansion is observed as compared to the expansion observed using a ratio of 1:1. In one particular aspect an increase of from about 1 to about 3 fold is observed as compared to the expansion observed using a ratio of 1:1. In one aspect, the ratio of CD3:CD28 antibody bound to the beads ranges from 100:1 to 1:100 and all integer values there between. In one aspect of the present invention, more anti-CD28 antibody is bound to the particles than anti-CD3 antibody, i.e., the ratio of CD3:CD28 is less than one. In certain aspects of the invention, the ratio of anti CD28 antibody to anti CD3 antibody bound to the beads is greater than 2:1. In one particular aspect, a 1:100 CD3:CD28 ratio of antibody bound to beads is used. In one aspect, a 1:75 CD3:CD28 ratio of antibody bound to beads is used. In a further aspect, a 1:50 CD3:CD28 ratio of antibody bound to beads is used. In one aspect, a 1:30 CD3:CD28 ratio of antibody bound to beads is used. In one preferred aspect, a 1:10 CD3:CD28 ratio of antibody bound to beads is used. In one aspect, a 1:3 CD3:CD28 ratio of antibody bound to the beads is used. In yet one aspect, a 3:1 CD3:CD28 ratio of antibody bound to the beads is used.

Ratios of particles to cells from 1:500 to 500:1 and any integer values in between may be used to stimulate T cells or other target cells. As those of ordinary skill in the art can readily appreciate, the ratio of particles to cells may depend on particle size relative to the target cell. For example, small sized beads could only bind a few cells, while larger beads could bind many. In certain aspects the ratio of cells to particles ranges from 1:100 to 100:1 and any integer values in-between and in further aspects the ratio comprises 1:9 to 9:1 and any integer values in between, can also be used to stimulate T cells. The ratio of anti-CD3- and anti-CD28-coupled particles to T cells that result in T cell stimulation can vary as noted above, however certain preferred values include 1:100, 1:50, 1:40, 1:30, 1:20, 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, 10:1, and 15:1 with one preferred ratio being at least 1:1 particles per T cell. In one aspect, a ratio of particles to cells of 1:1 or less is used. In one particular aspect, a preferred particle: cell ratio is 1:5. In further aspects, the ratio of particles to cells can be varied depending on the day of stimulation. For example, in one aspect, the ratio of particles to cells is from 1:1 to 10:1 on the first day and additional particles are added to the cells every day or every other day thereafter for up to 10 days, at final ratios of from 1:1 to 1:10 (based on cell counts on the day of addition). In one particular aspect, the ratio of particles to cells is 1:1 on the first day of stimulation and adjusted to 1:5 on the third and fifth days of stimulation. In one aspect, particles are added on a daily or every other day basis to a final ratio of 1:1 on the first day, and 1:5 on the third and fifth days of stimulation. In one aspect, the ratio of particles to cells is 2:1 on the first day of stimulation and adjusted to 1:10 on the third and fifth days of stimulation. In one aspect, particles are added on a daily or every other day basis to a final ratio of 1:1 on the first day, and 1:10 on the third and fifth days of stimulation. One of skill in the art will appreciate that a variety of other ratios may be suitable for use in the present invention. In particular, ratios will vary depending on particle size and on cell size and type. In one aspect, the most typical ratios for use are in the neighborhood of 1:1, 2:1 and 3:1 on the first day.

In further aspects of the present invention, the cells, such as T cells, are combined with agent-coated beads, the beads and the cells are subsequently separated, and then the cells are cultured. In an alternative aspect, prior to culture, the agent-coated beads and cells are not separated but are cultured together. In a further aspect, the beads and cells are first concentrated by application of a force, such as a magnetic force, resulting in increased ligation of cell surface markers, thereby inducing cell stimulation.

### Preparation of CoStAR Cells

Viral- and non-viral-based genetic engineering tools can be used to generate CoStAR cells, including without limitation T cells, NK cells resulting in permanent or transient expression of therapeutic genes. Retrovirus-based gene delivery is a mature, well-characterized technology, which has been used to permanently integrate CARs into the host cell genome (Scholler J., e.g. Decade-long safety and function of retroviral-modified chimeric antigen receptor T cells. Sci. Transl. Med. 2012; 4:132ra53; Rosenberg S. A. et al., Gene transfer into humans-immunotherapy of patients with advanced melanoma, using tumor-infiltrating lymphocytes modified by retroviral gene transduction. N. Engl. J. Med. 1990; 323:570-578)

Non-viral DNA transfection methods can also be used. For example, Singh et al describes use of the Sleeping Beauty (SB) transposon system developed to engineer CAR T cells (Singh H., et al., Redirecting specificity of T-cell populations for CD19 using the Sleeping Beauty system. Cancer Res. 2008; 68:2961-2971) and is being used in clinical trials (see e.g., ClinicalTrials.gov: NCT00968760 and NCT01653717). The same technology is applicable to engineer CoStARs cells.

Multiple SB enzymes have been used to deliver transgenes. Mátés describes a hyperactive transposase (SB100X) with approximately 100-fold enhancement in efficiency when compared to the first-generation transposase. SB100X supported 35-50% stable gene transfer in human CD34(+) cells enriched in hematopoietic stem or progenitor cells. (Mit6s L. et al., Molecular evolution of a novel hyperactive Sleeping Beauty transposase enables robust stable gene transfer in vertebrates. Nat. Genet. 2009; 41:753-761) and multiple transgenes can be delivered from multicistronic single plasmids (e.g., Thokala R. et al., Redirecting specificity of T cells using the Sleeping Beauty system to express chimeric antigen receptors by mix-and-matching of VL and VH domains targeting CD123+ tumors. PLoS ONE. 2016; 11:e0159477) or multiple plasmids (e.g., Hurton L. V. et al., Tethered IL-15 augments antitumor activity and promotes a stem-cell memory subset in tumor-specific T cells. Proc. Natl. Acad. Sci. USA. 2016; 113:E7788-E7797). Such systems are used with CoStARs of the invention.

Morita et al, describes the piggyBac transposon system to integrate larger transgenes (Morita D. et al., Enhanced expression of anti-CD19 chimeric antigen receptor in piggyBac transposon-engineered T cells. Mol. Ther. Methods Clin. Dev. 2017; 8:131-140) Nakazawa et al. describes use of the system to generate EBV-specific cytotoxic T-cells expressing HER2-specific chimeric antigen receptor (Nakazawa Y et al, PiggyBac-mediated cancer immunotherapy using EBV-specific cytotoxic T-cells expressing HER2-specific chimeric antigen receptor. Mol. Ther. 2011; 19:2133-2143). Manuri et al used the system to generate CD-19 specific T cells (Manuri P. V. R. et al., piggyBac transposon/transposase system to generate CD19-specific T cells for the treatment of B-lineage malignancies. Hum. Gene Ther. 2010; 21:427-437).

Transposon technology is easy and economical. One potential drawback is the longer expansion protocols currently employed may result in T cell differentiation, impaired activity and poor persistence of the infused cells. Monjezi et al describe development minicircle vectors that minimize these difficulties through higher efficiency integrations (Monjezi R. et al., Enhanced CAR T-cell engineering using non-viral Sleeping Beauty transposition from minicircle vectors. Leukemia. 2017; 31:186-194). These transposon technologies can be used for CoStARs of the invention.

### Pharmaceutical Compositions

The present invention also relates to a pharmaceutical composition containing a vector or a CoStAR expressing cell of the invention together with a pharmaceutically acceptable carrier, diluent or excipient, and optionally one or more further pharmaceutically active polypeptides and/or compounds.

In some embodiments, a pharmaceutical composition is provided comprising a CoStAR described above and a pharmaceutically acceptable carrier. In some embodiments, a pharmaceutical composition is provided comprising a nucleic acid encoding a CoStAR according to any of the embodiments described above and a pharmaceutically acceptable carrier. In some embodiments, a pharmaceutical composition is provided comprising an effector cell expressing a CoStAR described above and a pharmaceutically acceptable carrier. Such a formulation may, for example, be in a form suitable for intravenous infusion.

As used herein, by “pharmaceutically acceptable” or “pharmacologically compatible” is meant a material that is not biologically or otherwise undesirable, e.g., the material may be incorporated into a pharmaceutical composition administered to a patient without causing any significant undesirable biological effects or interacting in a deleterious manner with any of the other components of the composition in which it is contained. Pharmaceutically acceptable carriers or excipients have preferably met the required standards of toxicological and manufacturing testing and/or are included on the Inactive Ingredient Guide prepared by the U.S. Food and Drug administration.

An aspect of the invention provides a population of modified T cells expressing a recombinant CoStAR. A suitable population may be produced by a method described above.

The population of modified T cells may be for use as a medicament. For example, a population of modified T cells as described herein may be used in cancer immunotherapy therapy, for example adoptive T cell therapy.

Other aspects of the invention provide the use of a population of modified T cells as described herein for the manufacture of a medicament for the treatment of cancer, a population of modified T cells as described herein for the treatment of cancer, and a method of treatment of cancer may comprise administering a population of modified T cells as described herein to an individual in need thereof.

The population of modified T cells may be autologous i.e. the modified T cells were originally obtained from the same individual to whom they are subsequently administered (i.e. the donor and recipient individual are the same). A suitable population of modified T cells for administration to the individual may be produced by a method comprising providing an initial population of T cells obtained from the individual, modifying the T cells to express a cAMP PDE or fragment thereof and an antigen receptor which binds specifically to cancer cells in the individual, and culturing the modified T cells.

The population of modified T cells may be allogeneic i.e. the modified T cells were originally obtained from a different individual to the individual to whom they are subsequently administered (i.e. the donor and recipient individual are different). The donor and recipient individuals may be HLA matched to avoid GVHD and other undesirable immune effects. A suitable population of modified T cells for administration to a recipient individual may be produced by a method comprising providing an initial population of T cells obtained from a donor individual, modifying the T cells to express a CoStAR which binds specifically to cancer cells in the recipient individual, and culturing the modified T cells.

Following administration of the modified T cells, the recipient individual may exhibit a T cell mediated immune response against cancer cells in the recipient individual. This may have a beneficial effect on the cancer condition in the individual.

Cancer conditions may be characterized by the abnormal proliferation of malignant cancer cells and may include leukemias, such as AML, CML, ALL and CLL, lymphomas, such as Hodgkin lymphoma, non-Hodgkin lymphoma and multiple myeloma, and solid cancers such as sarcomas, skin cancer, melanoma, bladder cancer, brain cancer, breast cancer, uterus cancer, ovary cancer, prostate cancer, lung cancer, colorectal cancer, cervical cancer, liver cancer, head and neck cancer, oesophageal cancer, pancreas cancer, renal cancer, adrenal cancer, stomach cancer, testicular cancer, cancer of the gall bladder and biliary tracts, thyroid cancer, thymus cancer, cancer of bone, and cerebral cancer, as well as cancer of unknown primary (CUP).

Cancer cells within an individual may be immunologically distinct from normal somatic cells in the individual (i.e. the cancerous tumor may be immunogenic). For example, the cancer cells may be capable of eliciting a systemic immune response in the individual against one or more antigens expressed by the cancer cells. The tumor antigens that elicit the immune response may be specific to cancer cells or may be shared by one or more normal cells in the individual.

An individual suitable for treatment as described above may be a mammal, such as a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orangutan, gibbon), or a human.

In preferred embodiments, the individual is a human. In other preferred embodiments, non-human mammals, especially mammals that are conventionally used as models for demonstrating therapeutic efficacy in humans (e.g. murine, primate, porcine, canine, or rabbit animals) may be employed.

### Method of Treatment

The term “therapeutically effective amount” refers to an amount of a CoStAR or composition comprising a CoStAR as disclosed herein, effective to “treat” a disease or disorder in an individual. In the case of cancer, the therapeutically effective amount of a CoStAR or composition comprising a CoStAR as disclosed herein can reduce the number of cancer cells; reduce the tumor size or weight; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent a CoStAR or composition comprising a CoStAR as disclosed herein can prevent growth and/or kill existing cancer cells, it can be cytostatic and/or cytotoxic. In some embodiments, the therapeutically effective amount is a growth inhibitory amount. In some embodiments, the therapeutically effective amount is an amount that improves progression free survival of a patient. In the case of infectious disease, such as viral infection, the therapeutically effective amount of a CoStAR or composition comprising a CoStAR as disclosed herein can reduce the number of cells infected by the pathogen; reduce the production or release of pathogen-derived antigens; inhibit (i.e., slow to some extent and preferably stop) spread of the pathogen to uninfected cells; and/or relieve to some extent one or more symptoms associated with the infection. In some embodiments, the therapeutically effective amount is an amount that extends the survival of a patient.

Cells, including T and NK cells, expressing CoStARs for use in the methods of the present may either be created ex vivo either from a patient's own peripheral blood (autologous), or in the setting of a haematopoietic stem cell transplant from donor peripheral blood (allogenic), or peripheral blood from an unconnected donor (allogenic). Alternatively, T-cells or NK cells may be derived from ex-vivo differentiation of inducible progenitor cells or embryonic progenitor cells to T-cells or NK cells. In these instances, T-cells expressing a CoStAR and, optionally, a CAR and/or TCR, are generated by introducing DNA or RNA coding for the CoStAR and, optionally, a CAR and/or TCR, by one of many means including transduction with a viral vector, transfection with DNA or RNA.

T or NK cells expressing a CoStAR of the present invention and, optionally, expressing a TCR and/or CAR may be used for the treatment of haematological cancers or solid tumors.

A method for the treatment of disease relates to the therapeutic use of a vector or cell, including a T or NK cell, of the invention. In this respect, the vector, or T or NK cell may be administered to a subject having an existing disease or condition in order to lessen, reduce or improve at least one symptom associated with the disease and/or to slow down, reduce or block the progression of the disease. The method of the invention may cause or promote T-cell mediated killing of cancer cells. The vector, or T or NK cell according to the present invention may be administered to a patient with one or more additional therapeutic agents. The one or more additional therapeutic agents can be co-administered to the patient. By “co-administering” is meant administering one or more additional therapeutic agents and the vector, or T or NK cell of the present invention sufficiently close in time such that the vector, or T or NK cell can enhance the effect of one or more additional therapeutic agents, or vice versa. In this regard, the vectors or cells can be administered first and the one or more additional therapeutic agents can be administered second, or vice versa. Alternatively, the vectors or cells and the one or more additional therapeutic agents can be administered simultaneously. One co-administered therapeutic agent that may be useful is IL-2, as this is currently used in existing cell therapies to boost the activity of administered cells. However, IL-2 treatment is associated with toxicity and tolerability issues.

As mentioned, for administration to a patient, the CoStAR effector cells can be allogeneic or autologous to the patient. In some embodiments, allogeneic cells are further genetically modified, for example by gene editing, so as to minimize or prevent GVHD and/or a patient's immune response against the CoStAR cells.

The CoStAR effector cells are used to treat cancers and neoplastic diseases associated with a target antigen. Cancers and neoplastic diseases that may be treated using any of the methods described herein include tumors that are not vascularized, or not yet substantially vascularized, as well as vascularized tumors. The cancers may comprise non-solid tumors (such as hematological tumors, for example, leukemias and lymphomas) or may comprise solid tumors. Types of cancers to be treated with the CoStAR effector cells of the invention include, but are not limited to, carcinoma, blastoma, and sarcoma, and certain leukemia or lymphoid malignancies, benign and malignant tumors, and malignancies e.g., sarcomas, carcinomas, and melanomas. Adult tumors/cancers and pediatric tumors/cancers are also included.

Hematologic cancers are cancers of the blood or bone marrow. Examples of hematological (or hematogenous) cancers include leukemias, including acute leukemias (such as acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, plasmacytoma, Waldenstrom's macroglobulinemia, heavy chain disease, myelodysplastic syndrome, hairy cell leukemia and myelodysplasia.

Solid tumors are abnormal masses of tissue that usually do not contain cysts or liquid areas. Solid tumors can be benign or malignant. Different types of solid tumors are named for the type of cells that form them (such as sarcomas, carcinomas, and lymphomas). Examples of solid tumors, such as sarcomas and carcinomas, include adrenocortical carcinoma, cholangiocarcinoma, fibrosarcoma, myosarcoma, liposarcoma, chondrosarcoma, osteosarcoma, and other sarcomas, synovioma, mesothelioma, Ewing's tumor, leiomyosarcoma, rhabdomyosarcoma, colon carcinoma, stomach cancer, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, thyroid cancer (e.g., medullary thyroid carcinoma and papillary thyroid carcinoma), pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer (e.g., cervical carcinoma and pre-invasive cervical dysplasia), colorectal cancer, cancer of the anus, anal canal, or anorectum, vaginal cancer, cancer of the vulva (e.g., squamous cell carcinoma, intraepithelial carcinoma, adenocarcinoma, and fibrosarcoma), penile cancer, oropharyngeal cancer, esophageal cancer, head cancers (e.g., squamous cell carcinoma), neck cancers (e.g., squamous cell carcinoma), testicular cancer (e.g., seminoma, teratoma, embryonal carcinoma, teratocarcinoma, choriocarcinoma, sarcoma, Leydig cell tumor, fibroma, fibroadenoma, adenomatoid tumors, and lipoma), bladder carcinoma, kidney cancer, melanoma, cancer of the uterus (e.g., endometrial carcinoma), urothelial cancers (e.g., squamous cell carcinoma, transitional cell carcinoma, adenocarcinoma, ureter cancer, and urinary bladder cancer), and CNS tumors (such as a glioma (such as brainstem glioma and mixed gliomas), glioblastoma (also known as glioblastoma multiforme) astrocytoma, CNS lymphoma, germinoma, medulloblastoma, Schwannoma craniopharyogioma, ependymoma, pinealoma, hemangioblastoma, acoustic neuroma, oligodendroglioma, meningioma, neuroblastoma, retinoblastoma and brain metastases).

When “an immunologically effective amount,” “an anti-tumor effective amount,” “a tumor-inhibiting effective amount,” or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the T cells described herein may be administered at a dosage of 10⁴ to 10⁹ cells/kg body weight, in some instances 10⁵ to 10⁶ cells/kg body weight, including all integer values within those ranges. T cell compositions may also be administered multiple times at these dosages. The cells can be administered by using infusion techniques that are commonly known in immunotherapy (see, e.g., Rosenberg et al., New Eng. J. of Med. 319:1676, 1988).

### Combination Therapies

A CoStAR-expressing cell described herein may be used in combination with other known agents and therapies. Administered “in combination”, as used herein, means that two (or more) different treatments are delivered to the subject during the course of the subject's affliction with the disorder, e.g., the two or more treatments are delivered after the subject has been diagnosed with the disorder and before the disorder has been cured or eliminated or treatment has ceased for other reasons. In some embodiments, the delivery of one treatment is still occurring when the delivery of the second begins, so that there is overlap in terms of administration. This is sometimes referred to herein as “simultaneous” or “concurrent delivery”. In other embodiments, the delivery of one treatment ends before the delivery of the other treatment begins. In some embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

A CoStAR-expressing cell described herein and the at least one additional therapeutic agent can be administered simultaneously, in the same or in separate compositions, or sequentially. For sequential administration, the CAR-expressing cell described herein can be administered first, and the additional agent can be administered second, or the order of administration can be reversed.

The CoStAR therapy and/or other therapeutic agents, procedures or modalities can be administered during periods of active disorder, or during a period of remission or less active disease. The CoStAR therapy can be administered before the other treatment, concurrently with the treatment, post-treatment, or during remission of the disorder.

When administered in combination, the therapy and the additional agent (e.g., second or third agent), or all, can be administered in an amount or dose that is higher, lower or the same than the amount or dosage of each agent used individually, e.g., as a monotherapy. In some embodiments, the administered amount or dosage of the CoStAR therapy, the additional agent (e.g., second or third agent), or all, is lower (e.g., at least 20%, at least 30%, at least 40%, or at least 50%) than the amount or dosage of each agent used individually, e.g., as a monotherapy. In other embodiments, the amount or dosage of the CoStAR therapy, the additional agent (e.g., second or third agent), or all, that results in a desired effect (e.g., treatment of cancer) is lower (e.g., at least 20%, at least 30%, at least 40%, or at least 50% lower) than the amount or dosage of each agent used individually, e.g., as a monotherapy, required to achieve the same therapeutic effect.

In further aspects, a CoStAR-expressing cell described herein may be used in a treatment regimen in combination with surgery, chemotherapy, radiation, immunosuppressive agents, such as cyclosporin, azathioprine, methotrexate, mycophenolate, and FK506, antibodies, or other immunoablative agents such as CAMPATH, anti-CD3 antibodies or other antibody therapies, cytoxin, fludarabine, cyclosporin, FK506, rapamycin, mycophenolic acid, steroids, FR901228, cytokines, and irradiation, peptide vaccine, such as that described in Izumoto et al. 2008 J Neurosurg 108:963-971.

In certain instances, compounds of the present invention are combined with other therapeutic agents, such as other anti-cancer agents, anti-allergic agents, anti-nausea agents (or anti-emetics), pain relievers, cytoprotective agents, and combinations thereof.

In one embodiment, a CoStAR-expressing cell described herein can be used in combination with a chemotherapeutic agent. Exemplary chemotherapeutic agents include an anthracycline (e.g., doxorubicin (e.g., liposomal doxorubicin)), a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, ofatumumab, tositumomab, brentuximab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide).

General Chemotherapeutic agents considered for use in combination therapies include busulfan (Myleran®), busulfan injection (Busulfex®), cladribine (Leustatin®), cyclophosphamide (Cytoxan® or Neosar®), cytarabine, cytosine arabinoside (Cytosar-U®), cytarabine liposome injection (DepoCyt®), daunorubicin hydrochloride (Cerubidine®), daunorubicin citrate liposome injection (DaunoXome®), dexamethasone, doxorubicin hydrochloride (Adriamycin®, Rubex®), etoposide (Vepesid®), fludarabine phosphate (Fludara®), hydroxyurea (Hydrea®), Idarubicin (Idamycin®), mitoxantrone (Novantrone®), Gemtuzumab Ozogamicin (Mylotarg®).

In embodiments, general chemotherapeutic agents considered for use in combination therapies include anastrozole (Arimidex®), bicalutamide (Casodex®), bleomycin sulfate (Blenoxane®), busulfan (Myleran®), busulfan injection (Busulfex®), capecitabine (Xeloda®), N4-pentoxycarbonyl-5-deoxy-5-fluorocytidine, carboplatin (Paraplatin®), carmustine (BiCNU®), chlorambucil (Leukeran®), cisplatin (Platinol®), cladribine (Leustatin®), cyclophosphamide (Cytoxan® or Neosar®), cytarabine, cytosine arabinoside (Cytosar-U®), cytarabine liposome injection (DepoCyt®), dacarbazine (DTIC-Dome®), dactinomycin (Actinomycin D, Cosmegan), daunorubicin hydrochloride (Cerubidine®), daunorubicin citrate liposome injection (DaunoXome®), dexamethasone, docetaxel (Taxotere®), doxorubicin hydrochloride (Adriamycin®, Rubex®), etoposide (Vepesid®), fludarabine phosphate (Fludara®), 5-fluorouracil (Adrucil®, Efudex®), flutamide (Eulexin®), tezacitibine, Gemcitabine (difluorodeoxycitidine), hydroxyurea (Hydrea®), Idarubicin (Idamycin®), ifosfamide (IFEX®), irinotecan (Camptosar®), L-asparaginase (ELSPAR®), leucovorin calcium, melphalan (Alkeran®), 6-mercaptopurine (Purinethol®), methotrexate (Folex®), mitoxantrone (Novantrone®), mylotarg, paclitaxel (Taxol®), phoenix (Yttrium90/MX-DTPA), pentostatin, polifeprosan 20 with carmustine implant (Gliadel®), tamoxifen citrate (Nolvadex®), teniposide (Vumon®), 6-thioguanine, thiotepa, tirapazamine (Tirazone®), topotecan hydrochloride for injection (Hycamptin®), vinblastine (Velban®), vincristine (Oncovin®), and vinorelbine (Navelbine®).

Treatments can be evaluated, for example, by tumor regression, tumor weight or size shrinkage, time to progression, duration of survival, progression free survival, overall response rate, duration of response, quality of life, protein expression and/or activity. Approaches to determining efficacy of the therapy can be employed, including for example, measurement of response through radiological imaging.

### Sequences

The following sequences include complete CoStARs and CoStAR components and are non-limiting. Components include signal peptides (SP), binding domains (BD), linkers, spacers and transmembrane domains (STM), a CD28 transmembrane fragment without extracellular or intracellular sequences (STM-CD28TM), intracellular signal domains (SD) and CD40 domains and motifs. Whereas SEQ ID NOS:42-247 comprise CoStARs with N-terminal signal peptides, it will be understood that the N-terminal signal peptides are removed from a mature CoStAR. Further, is will be understood that a mature CoStAR may lack 1, 2, 3, 4, 5, 6, 7, 8, 9, or 10 or more amino acids at the N-terminal (i.e., counted from the C-terminal end of the signal peptide). Component locations within whole proteins can be confirmed from GenBank and other sources. The constructs and components are illustrative as to precise sizes and extents and components can be from more than one source. Where there is more than one intracellular signaling domain or signaling fragment, the multiple domains can be in any order. It will be understood that whereas certain proteins may comprise N-terminal signal peptides when expressed, those signal peptides are cleaved and may be imprecisely cleaved when the proteins are expressed, and that the resulting proteins from which signal peptides are removed comprise binding domains having variation of up to about five amino acids in the location of the N-terminal amino acid.

TABLE 7
Amino Acid Sequences

| ID | | |
|---|---|---|
| No | Component | Sequence |
| 1 | SP-OSM | MGVLLTQRTL LSLVLALLFP SMSAM |
| 2 | SP-CD8α | MALPVTALLL PLALLLHAAR P |
| 3 | SP-CD2 | MSFPCKFVAS FLLIFNVSSK GAVS |
| 4 | SP-IL2 | MYRMQLLSCI ALSLALVTNS |
| 5 | SP-GM-CSF | MWLQSLLLLG TVACSIS |
| 6 | SP-hIgGk-VIII | MEAPAQLLFL LLLWLPDTTR |
| 7 | SP-PD1 | MGVLLTQRTL LSLVLALLFP SMASM |
| 8 | SP-TIGIT | MQIPQAPWPV VWAVLQLGWR PGW |
| 9 | BD1-MOV | QVQLQQSGAE LVKPGASVKI SCKASGYSFT GYFMNWVKQS HGKSLEWIGR |
| | | IHPYDGDTFY NQNFKDKATL TVDKSSNTAH MELLSLTSED FAVYYCTRYD |
| | | GSRAMDYWGQ GTTVTVSSGG GGSGGGGSGG GGSDIELTQS PASLAVSLGQ |
| | | RAIISCKASQ SVSFAGTSLM HWYHQKPGQQ PKLLIYRASN LEAGVPTRFS |
| | | GSGSKTDFTL NIHPVEEEDA ATYYCQQSRE YPYTFGGGTK LEIK |
| 10 | MFE23 | QVKLQQSGAE LVRSGTSVKL SCTASGFNIK DSYMHWLRQG PEQGLEWIGW |
| | | IDPENGDTEY APKFQGKATF TTDTSSNTAY LQLSSLTSED TAVYYCNEGT |
| | | PTGPYYFDYW GQGTTVTVSS GGGGSGGGGS GGGGSENVLT QSPAIMSASP |
| | | GEKVTITCSA SSSVSYMHWF QQKPGTSPKL WIYSTSNLAS GVPARFSGSG |
| | | SGTSYSLTIS RMEAEDAATY YCQQRSSYPL TFGAGTKLEL KR |
| 11 | MFE23 | QVQLQQSGAE LVRSGTSVKL SCTASGFNIK DSYMHWLRQG PEQGLEWIGW |
| | (K > Q) | IDPENGDTEY APKFQGKATF TTDTSSNTAY LQLSSLTSED TAVYYCNEGT |
| | | PTGPYYFDYW GQGTTVTVSS GGGGSGGGGS GGGGSENVLT QSPAIMSASP |
| | | GEKVTITCSA SSSVSYMHWF QQKPGTSPKL WIYSTSNLAS GVPARFSGSG |
| | | SGTSYSLTIS RMEAEDAATY YCQQRSSYPL TFGAGTKLEL KR |
| 12 | hMFE23 | QVKLEQSGAE VVKPGASVKL SCKASGFNIK DSYMHWLRQG PGQRLEWIGW |
| | | IDPENGDTEY APKFQGKATF TTDTSANTAY LGLSSLRPED TAVYYCNEGT |
| | | PTGPYYFDYW GQGTLVTVSS GGGGSGGGGS GGGGSENVLT QSPSSMSASV |
| | | GDRVNIACSA SSSVSYMHWF QQKPGKSPKL WIYSTSNLAS GVPSRFSGSG |
| | | SGTDYSLTIS SMQPEDAATY YCQQRSSYPL TFGGGTKLEI K |
| 13 | CEA6 | QVQLVQSGAE VKKPGSSVKV SCKASGGTFS NSPINWLRQA PGQGLEWMGS |
| | | IIPSFGTANY AQKFQGRLTI TADESTSTAY MELSSLRSED TAVYYCAGRS |
| | | HNYELYYYYM DVWGQGTMVT VSSGGGGSGG GGSGGGGSDI QMTQSPSTLS |
| | | ASIGDRVTIT CRASEGIYHW LAWYQQKPGK APKLLIYKAS SLASGAPSRF |
| | | SGSGSGTDFT LTISSLQPDD FATYYCQQYS NYPLTFGGGT KLEIKR |
| 14 | BW431/26 | QLQESGPGLV RPSQTLSLTC TVSGFTISSG YSWHWVRQPP GRGLEWIGYI |
| | | QYSGITNYNP SLKSRVTMLV DTSKNQFSLR LSSVTAADTA VYYCAREDYD |
| | | YHWYFDVWGQ GSLVTVSSGG GGSGGGGSGG GGSGVHSDIQ MTQSPSSLSA |
| | | SVGDRVTITC STSSSVSYMH WYQQKPGKAP KLLIYSTSNL ASGVPSRFSG |
| | | SGSGTDFTFT ISSLQPEDIA TYYCHQWSSY PTFGQGTKVE IKR |
| 15 | HuT84.66 | EVQLVESGGG LVQPGGSLRL SCAASGFNIK DTYMHWVRQA PGKGLEWVAR |
| | (M5A) | IDPANGNSKY ADSVKGRFTI SADTSKNTAY LQMNSLRAED TAVYYCAPFG |
| | | YYVSDYAMAY WGQGTLVTVS SGGGGSGGGG SGGGGSDIQL TQSPSSLSAS |
| | | VGDRVTITCR AGESVDIFGV GFLHWYQQKP GKAPKLLIYR ASNLESGVPS |
| | | RFSGSGSRTD FTLTISSLQP EDFATYYCQQ TNEDPYTFGQ GTKVEIK |
| 16 | BD1-PD1 | RPGWFLDSPD RPWNPPTFSP ALLVVTEGDN ATFTCSFSNT SESFVLNWYR |
| | | MSPSNQTDKL AAFPEDRSQP GQDCRFRVTQ LPNGRDFHMS VVRARRNDSG |
| | | TYLCGAISLA PKAQIKESLR AELRVTERRA EVPTAH |
| 17 | BD1-TIGIT | MMTGTIETTG NISAEKGGSI ILQCHLSSTT AQVTQVNWEQ QDQLLAICNA |
| | | DLGWHISPSF KDRVAPGPGL GLTLQSLTVN DTGEYFCIYH TYPDGTYTGR |
| | | IFLEVLESSV AEHGARFQIP |
| 18 | 3XA2Xgsg | AAAGSGGSG |
| 19 | STM-spCD28 | ILVKQSPMLV AYDNAVNLSC KYSYNLFSRE FRASLHKGLD SAVEVCVVYG |
| | | NYSQQLQVYS KTGFNCDGKL GNESVTFYLQ NLYVNQTDIY FCKIEVMYPP |
| | | PYLDNEKSNG TIIHVKGKHL CPSPLFPGPS KPFWVLVVVG GVLACYSLLV |
| | | TVAFIIFWV |
| 20 | STM-spCD8 | FVPVFLPAKP TTTPAPRPPT PAPTIASQPL SLRPEACRPA AGGAVHTRGL |
| | | DFACDIYIWA PLAGTCGVLL LSLVITLYCN HRN |
| 21 | STM-CD28TM | FWVLVVVGGV LACYSLLVTV AFIIFWV |
| 22 | STM-spCD28TM | CPSPLFPGPS KPFWVLVVVG GVLACYSLLV TVAFIIFWV |
| 23 | STM- | IIHVKGKHLC PSPLFPGPSK PFWVLVVVGG VLACYSLLVT VAFIIFWV |
| | CD28IIH (trun) | |
| 24 | STM-spIG4 | ESKYGPPCPS CPAPEFLGGP SVFLFPPKPK DTLMISRTPE VTCVVVDVSQ |
| | | EDPEVQFNWY VDGVEVHNAK TKPREEQFNS TYRVVSVLTV LHQDWLNGKE |
| | | YKCKVSNKGL PSSIEKTISK AKGQPREPQV YTLPPSQEEM TKNQVSLTCL |
| | | VKGFYPSDIA VEWESNGQPE NNYKTTPPVL DSDGSFFLYS RLTVDKSRWQ |
| | | EGNVFSCSVM HEALHNHYTQ KSLSLSLGKM FWVLVVVGGV LACYSLLVTV |
| | | AFIIFWV |
| 25 | Sig-CD28 | RSKRSRLLHS DYMNMTPRRP GPTRKHYQPY APPRDFAAYR S |
| 26 | Sig-CD137 | RFSVVKRGRK KLLYIFKQPF MRPVQTTQEE DGCSCRFPEE EEGGCE |
| 27 | Sig-CD134 | RRDQRLPPDA HKPPGGGSFR TPIQEEQADA HSTLAKI |
| 28 | Sig-CD2 | KRKKQRSRRN DEELETRAHR VATEERGRKP HQIPASTPQN PATSQHPPPP |
| | | PGHRSQAPSH RPPPPGHRVQ HQPQKRPPAP SGTQVHQQKG PPLPRPRVQP |
| | | KPPHGAAENS LSPSSN |
| 29 | Sig GITR | QLGLHIWQLR SQCMWPRETQ LLLEVPPSTE DARSCQFPEE ERGERSAEEK |
| | | GRLGDLWV |
| 30 | Sig-CD29 | KLLMIIHDRR EFAKFEKEKM NAKWDTGENP IYKSAVTTVV NPKYEGK |
| 31 | Sig-CD150 | RRRGKTNHYQ TTVEKKSLTI YAQVQKPGPL QKKLDSFPAQ DPCTTIYVAA |
| | | TEPVPESVQE TNSITVYASV TLPES |
| 32 | CD40 | KKVAKKPTNK APHPKQEPQE INFPDDLPGS NTAAPVQETL HGCQPVTQED |
| | | GKESRISVQE RQ |
| 33 | CD40_tandem | AKKPTNKAPH PKQEPQEINF PDDLPGSNTA APVQETLHGC QPVTQEDGKE |
| | | SRISVQERQK KVAKKPTNKA PHPKQEPQEI NFPDDLPGSN TAAPVQETLH |
| | | GCQPVTQEDG KESRISVQER QKKVA |
| 34 | CD40_P227A | KKVAKKPTNK AAHPKQEPQE INFPDDLPGS NTAAPVQETL HGCQPVTQED |
| | | GKESRISVQE RQ |
| 35 | SH3_motif | KPTNKAPH |
| 36 | TRAF2_motif1 | PKQE |
| 37 | TRAF2_motif2 | PVQE |
| 38 | TRAF2_motif3 | SVQE |
| 39 | TRAF6_motif | QEPQEINFP |
| 40 | PKA_motif1 | KKPTNKA |
| 41 | PKA_motif2 | SRISVQE |
| 42 | OSM_MFE23_ | MGVLLTQRTL LSLVLALLFP SMASMQVKLQ QSGAELVRSG TSVKLSCTAS |
| | spCD28_ | GFNIKDSYMH WLRQGPEQGL EWIGWIDPEN GDTEYAPKFQ GKATFTTDTS |
| | CD28_CD40 | SNTAYLQLSS LTSEDTAVYY CNEGTPTGPY YFDYWGQGTT VTVSSGGGGS |
| | CTP194 | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGILVK QSPMLVAYDN AVNLSCKYSY |
| | | NLFSREFRAS LHKGLDSAVE VCVVYGNYSQ QLQVYSKTGF NCDGKLGNES |
| | | VTFYLQNLYV NQTDIYFCKI EVMYPPPYLD NEKSNGTIIH VKGKHLCPSP |
| | | LFPGPSKPFW VLVVVGGVLA CYSLLVTVAF IIFWVRSKRS RLLHSDYMNM |
| | | TPRRPGPTRK HYQPYAPPRD FAAYRSKKVA KKPTNKAPHP KQEPQEINFP |
| | | DDLPGSNTAA PVQETLHGCQ PVTQEDGKES RISVQERQ |
| 43 | CD8α_MFE23_ | MALPVTALLL PLALLLHAAR PQVKLQQSGA ELVRSGTSVK LSCTASGFNI |
| | spCD28_ | KDSYMHWLRQ GPEQGLEWIG WIDPENGDTE YAPKFQGKAT FTTDTSSNTA |
| | CD28_CD40 | YLQLSSLTSE DTAVYYCNEG TPTGPYYFDY WGQGTTVTVS SGGGGSGGGG |
| | CTP255 | SGGGGSENVL TQSPAIMSAS PGEKVTITCS ASSSVSYMHW FQQKPGTSPK |
| | | LWIYSTSNLA SGVPARFSGS GSGTSYSLTI SRMEAEDAAT YYCQQRSSYP |
| | | LTFGAGTKLE LKRAAAGSGG SGILVKQSPM LVAYDNAVNL SCKYSYNLFS |
| | | REFRASLHKG LDSAVEVCVV YGNYSQQLQV YSKTGFNCDG KLGNESVTFY |
| | | LQNLYVNQTD IYFCKIEVMY PPPYLDNEKS NGTIIHVKGK HLCPSPLFPG |
| | | PSKPFWVLVV VGGVLACYSL LVTVAFIIFW VRSKRSRLLH SDYMNMTPRR |
| | | PGPTRKHYQP YAPPRDFAAY RSKKVAKKPT NKAPHPKQEP QEINFPDDLP |
| | | GSNTAAPVQE TLHGCQPVTQ EDGKESRISV QERQ |
| 44 | CD2_MFE23_ | MSFPCKFVAS FLLIFNVSSK GAVSQVKLQQ SGAELVRSGT SVKLSCTASG |
| | spCD28_ | FNIKDSYMHW LRQGPEQGLE WIGWIDPENG DTEYAPKFQG KATFTTDTSS |
| | CD28_CD40 | NTAYLQLSSL TSEDTAVYYC NEGTPTGPYY FDYWGQGTTV TVSSGGGGSG |
| | CTP256 | GGGSGGGGSE NVLTQSPAIM SASPGEKVTI TCSASSSVSY MHWFQQKPGT |
| | | SPKLWIYSTS NLASGVPARF SGSGSGTSYS LTISRMEAED AATYYCQQRS |
| | | SYPLTFGAGT KLELKRAAAG SGGSGILVKQ SPMLVAYDNA VNLSCKYSYN |
| | | LFSREFRASL HKGLDSAVEV CVVYGNYSQQ LQVYSKTGFN CDGKLGNESV |
| | | TFYLQNLYVN QTDIYFCKIE VMYPPPYLDN EKSNGTIIHV KGKHLCPSPL |
| | | FPGPSKPFWV LVVVGGVLAC YSLLVTVAFI IFWVRSKRSR LLHSDYMNMT |
| | | PRRPGPTRKH YQPYAPPRDF AAYRSKKVAK KPTNKAPHPK QEPQEINFPD |
| | | DLPGSNTAAP VQETLHGCQP VTQEDGKESR ISVQERQ |
| 45 | IL2_MFE23_ | MYRMQLLSCI ALSLALVTNS QVKLQQSGAE LVRSGTSVKL SCTASGFNIK |
| | spCD28_ | DSYMHWLRQG PEQGLEWIGW IDPENGDTEY APKFQGKATF TTDTSSNTAY |
| | CD28_CD40 | LQLSSLTSED TAVYYCNEGT PTGPYYFDYW GQGTTVTVSS GGGGSGGGGS |
| | CTP257 | GGGGSENVLT QSPAIMSASP GEKVTITCSA SSSVSYMHWF QQKPGTSPKL |
| | | WIYSTSNLAS GVPARFSGSG SGTSYSLTIS RMEAEDAATY YCQQRSSYPL |
| | | TFGAGTKLEL KRAAAGSGGS GILVKQSPML VAYDNAVNLS CKYSYNLFSR |
| | | EFRASLHKGL DSAVEVCVVY GNYSQQLQVY SKTGFNCDGK LGNESVTFYL |
| | | QNLYVNQTDI YFCKIEVMYP PPYLDNEKSN GTIIHVKGKH LCPSPLFPGP |
| | | SKPFWVLVVV GGVLACYSLL VTVAFIIFWV RSKRSRLLHS DYMNMTPRRP |
| | | GPTRKHYQPY APPRDFAAYR SKKVAKKPTN KAPHPKQEPQ EINFPDDLPG |
| | | SNTAAPVQET LHGCQPVTQE DGKESRISVQ ERQ |
| 46 | GM-CSF_MFE23_ | MWLQSLLLLG TVACSISQVK LQQSGAELVR SGTSVKLSCT ASGFNIKDSY |
| | spCD28_ | MHWLRQGPEQ GLEWIGWIDP ENGDTEYAPK FQGKATFTTD TSSNTAYLQL |
| | CD28_CD40 | SSLTSEDTAV YYCNEGTPTG PYYFDYWGQG TTVTVSSGGG GSGGGGSGGG |
| | CTP258 | GSENVLTQSP AIMSASPGEK VTITCSASSS VSYMHWFQQK PGTSPKLWIY |
| | | STSNLASGVP ARFSGSGSGT SYSLTISRME AEDAATYYCQ QRSSYPLTFG |
| | | AGTKLELKRA AAGSGGSGIL VKQSPMLVAY DNAVNLSCKY SYNLFSREFR |
| | | ASLHKGLDSA VEVCVVYGNY SQQLQVYSKT GFNCDGKLGN ESVTFYLQNL |
| | | YVNQTDIYFC KIEVMYPPPY LDNEKSNGTI IHVKGKHLCP SPLFPGPSKP |
| | | FWVLVVVGGV LACYSLLVTV AFIIFWVRSK RSRLLHSDYM NMTPRRPGPT |
| | | RKHYQPYAPP RDFAAYRSKK VAKKPTNKAP HPKQEPQEIN FPDDLPGSNT |
| | | AAPVQETLHG CQPVTQEDGK ESRISVQERQ |
| 47 | hIgGk- | MEAPAQLLFL LLLWLPDTTR QVKLQQSGAE LVRSGTSVKL SCTASGFNIK |
| | VIII_MFE23_ | DSYMHWLRQG PEQGLEWIGW IDPENGDTEY APKFQGKATF TTDTSSNTAY |
| | spCD28_ | LQLSSLTSED TAVYYCNEGT PTGPYYFDYW GQGTTVTVSS GGGGSGGGGS |
| | CD28_CD40 | GGGGSENVLT QSPAIMSASP GEKVTITCSA SSSVSYMHWF QQKPGTSPKL |
| | CTP259 | WIYSTSNLAS GVPARFSGSG SGTSYSLTIS RMEAEDAATY YCQQRSSYPL |
| | | TFGAGTKLEL KRAAAGSGGS GILVKQSPML VAYDNAVNLS CKYSYNLFSR |
| | | EFRASLHKGL DSAVEVCVVY GNYSQQLQVY SKTGFNCDGK LGNESVTFYL |
| | | QNLYVNQTDI YFCKIEVMYP PPYLDNEKSN GTIIHVKGKH LCPSPLFPGP |
| | | SKPFWVLVVV GGVLACYSLL VTVAFIIFWV RSKRSRLLHS DYMNMTPRRP |
| | | GPTRKHYQPY APPRDFAAYR SKKVAKKPTN KAPHPKQEPQ EINFPDDLPG |
| | | SNTAAPVQET LHGCQPVTQE DGKESRISVQ ERQ |
| 48 | OSM_MFE23_ | MGVLLTQRTL LSLVLALLFP SMASMQVKLQ QSGAELVRSG TSVKLSCTAS |
| | spCD8_ | GFNIKDSYMH WLRQGPEQGL EWIGWIDPEN GDTEYAPKFQ GKATFTTDTS |
| | CD28_CD40 | SNTAYLQLSS LTSEDTAVYY CNEGTPTGPY YFDYWGQGTT VTVSSGGGGS |
| | CTP190 | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGFVPV FLPAKPTTTP APRPPTPAPT |
| | | IASQPLSLRP EACRPAAGGA VHTRGLDFAC DIYIWAPLAG TCGVLLLSLV |
| | | ITLYCNHRNR SKRSRLLHSD YMNMTPRRPG PTRKHYQPYA PPRDFAAYRS |
| | | KKVAKKPTNK APHPKQEPQE INFPDDLPGS NTAAPVQETL HGCQPVTQED |
| | | GKESRISVQE RQ |
| 49 | CD8a_MFE23_ | MALPVTALLL PLALLLHAAR PQVKLQQSGA ELVRSGTSVK LSCTASGFNI |
| | spCD8_ | KDSYMHWLRQ GPEQGLEWIG WIDPENGDTE YAPKFQGKAT FTTDTSSNTA |
| | CD28_CD40 | YLQLSSLTSE DTAVYYCNEG TPTGPYYFDY WGQGTTVTVS SGGGGSGGGG |
| | | SGGGGSENVL TQSPAIMSAS PGEKVTITCS ASSSVSYMHW FQQKPGTSPK |
| | | LWIYSTSNLA SGVPARFSGS GSGTSYSLTI SRMEAEDAAT YYCQQRSSYP |
| | | LTFGAGTKLE LKRAAAGSGG SGFVPVFLPA KPTTTPAPRP PTPAPTIASQ |
| | | PLSLRPEACR PAAGGAVHTR GLDFACDIYI WAPLAGTCGV LLLSLVITLY |
| | | CNHRNRSKRS RLLHSDYMNM TPRRPGPTRK HYQPYAPPRD FAAYRSKKVA |
| | | KKPTNKAPHP KQEPQEINFP DDLPGSNTAA PVQETLHGCQ PVTQEDGKES |
| | | RISVQERQ |
| 50 | CD2_MFE23_ | MSFPCKFVAS FLLIFNVSSK GAVSQVKLQQ SGAELVRSGT SVKLSCTASG |
| | spCD8_ | FNIKDSYMHW LRQGPEQGLE WIGWIDPENG DTEYAPKFQG KATFTTDTSS |
| | CD28_CD40 | NTAYLQLSSL TSEDTAVYYC NEGTPTGPYY FDYWGQGTTV TVSSGGGGSG |
| | | GGGSGGGGSE NVLTQSPAIM SASPGEKVTI TCSASSSVSY MHWFQQKPGT |
| | | SPKLWIYSTS NLASGVPARF SGSGSGTSYS LTISRMEAED AATYYCQQRS |
| | | SYPLTFGAGT KLELKRAAAG SGGSGFVPVF LPAKPTTTPA PRPPTPAPTI |
| | | ASQPLSLRPE ACRPAAGGAV HTRGLDFACD IYIWAPLAGT CGVLLLSLVI |
| | | TLYCNHRNRS KRSRLLHSDY MNMTPRRPGP TRKHYQPYAP PRDFAAYRSK |
| | | KVAKKPTNKA PHPKQEPQEI NFPDDLPGSN TAAPVQETLH GCQPVTQEDG |
| | | KESRISVQER Q |
| 51 | IL2_MFE23_ | MYRMQLLSCI ALSLALVTNS QVKLQQSGAE LVRSGTSVKL SCTASGFNIK |
| | spCD8_ | DSYMHWLRQG PEQGLEWIGW IDPENGDTEY APKFQGKATF TTDTSSNTAY |
| | CD28_CD40 | LQLSSLTSED TAVYYCNEGT PTGPYYFDYW GQGTTVTVSS GGGGSGGGGS |
| | | GGGGSENVLT QSPAIMSASP GEKVTITCSA SSSVSYMHWF QQKPGTSPKL |
| | | WIYSTSNLAS GVPARFSGSG SGTSYSLTIS RMEAEDAATY YCQQRSSYPL |
| | | TFGAGTKLEL KRAAAGSGGS GFVPVFLPAK PTTTPAPRPP TPAPTIASQP |
| | | LSLRPEACRP AAGGAVHTRG LDFACDIYIW APLAGTCGVL LLSLVITLYC |
| | | NHRNRSKRSR LLHSDYMNMT PRRPGPTRKH YQPYAPPRDF AAYRSKKVAK |
| | | KPTNKAPHPK QEPQEINFPD DLPGSNTAAP VQETLHGCQP VTQEDGKESR |
| | | ISVQERQ |
| 52 | GM-CSF_MFE23_ | MWLQSLLLLG TVACSISQVK LQQSGAELVR SGTSVKLSCT ASGFNIKDSY |
| | spCD8_ | MHWLRQGPEQ GLEWIGWIDP ENGDTEYAPK FQGKATFTTD TSSNTAYLQL |
| | CD28_CD40 | SSLTSEDTAV YYCNEGTPTG PYYFDYWGQG TTVTVSSGGG GSGGGGSGGG |
| | | GSENVLTQSP AIMSASPGEK VTITCSASSS VSYMHWFQQK PGTSPKLWIY |
| | | STSNLASGVP ARFSGSGSGT SYSLTISRME AEDAATYYCQ QRSSYPLTFG |
| | | AGTKLELKRA AAGSGGSGFV PVFLPAKPTT TPAPRPPTPA PTIASQPLSL |
| | | RPEACRPAAG GAVHTRGLDF ACDIYIWAPL AGTCGVLLLS LVITLYCNHR |
| | | NRSKRSRLLH SDYMNMTPRR PGPTRKHYQP YAPPRDFAAY RSKKVAKKPT |
| | | NKAPHPKQEP QEINFPDDLP GSNTAAPVQE TLHGCQPVTQ EDGKESRISV |
| | | QERQ |
| 53 | hIgGk- | MEAPAQLLFL LLLWLPDTTR QVKLQQSGAE LVRSGTSVKL SCTASGFNIK |
| | VIII_MFE23_ | DSYMHWLRQG PEQGLEWIGW IDPENGDTEY APKFQGKATF TTDTSSNTAY |
| | spCD8_ | LQLSSLTSED TAVYYCNEGT PTGPYYFDYW GQGTTVTVSS GGGGSGGGGS |
| | CD28_CD40 | GGGGSENVLT QSPAIMSASP GEKVTITCSA SSSVSYMHWF QQKPGTSPKL |
| | | WIYSTSNLAS GVPARFSGSG SGTSYSLTIS RMEAEDAATY YCQQRSSYPL |
| | | TFGAGTKLEL KRAAAGSGGS GFVPVFLPAK PTTTPAPRPP TPAPTIASQP |
| | | LSLRPEACRP AAGGAVHTRG LDFACDIYIW APLAGTCGVL LLSLVITLYC |
| | | NHRNRSKRSR LLHSDYMNMT PRRPGPTRKH YQPYAPPRDF AAYRSKKVAK |
| | | KPTNKAPHPK QEPQEINFPD DLPGSNTAAP VQETLHGCQP VTQEDGKESR |
| | | ISVQERQ |
| 54 | OSM_MFE23_ | MGVLLTQRTL LSLVLALLFP SMASMQVKLQ QSGAELVRSG TSVKLSCTAS |
| | CD28TM_ | GFNIKDSYMH WLRQGPEQGL EWIGWIDPEN GDTEYAPKFQ GKATFTTDTS |
| | CD28_CD40 | SNTAYLQLSS LTSEDTAVYY CNEGTPTGPY YFDYWGQGTT VTVSSGGGGS |
| | | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGFWVL VVVGGVLACY SLLVTVAFII |
| | | FWVRSKRSRL LHSDYMNMTP RRPGPTRKHY QPYAPPRDFA AYRSKKVAKK |
| | | PTNKAPHPKQ EPQEINFPDD LPGSNTAAPV QETLHGCQPV TQEDGKESRI |
| | | SVQERQ |
| 55 | CD8a_MFE23_ | MALPVTALLL PLALLLHAAR PQVKLQQSGA ELVRSGTSVK LSCTASGFNI |
| | CD28TM_ | KDSYMHWLRQ GPEQGLEWIG WIDPENGDTE YAPKFQGKAT FTTDTSSNTA |
| | CD28_CD40 | YLQLSSLTSE DTAVYYCNEG TPTGPYYFDY WGQGTTVTVS SGGGGSGGGG |
| | | SGGGGSENVL TQSPAIMSAS PGEKVTITCS ASSSVSYMHW FQQKPGTSPK |
| | | LWIYSTSNLA SGVPARFSGS GSGTSYSLTI SRMEAEDAAT YYCQQRSSYP |
| | | LTFGAGTKLE LKRAAAGSGG SGFWVLVVVG GVLACYSLLV TVAFIIFWVR |
| | | SKRSRLLHSD YMNMTPRRPG PTRKHYQPYA PPRDFAAYRS KKVAKKPTNK |
| | | APHPKQEPQE INFPDDLPGS NTAAPVQETL HGCQPVTQED GKESRISVQE |
| | | RQ |
| 56 | CD2_MFE23_ | MSFPCKFVAS FLLIFNVSSK GAVSQVKLQQ SGAELVRSGT SVKLSCTASG |
| | CD28TM_ | FNIKDSYMHW LRQGPEQGLE WIGWIDPENG DTEYAPKFQG KATFTTDTSS |
| | CD28_CD40 | NTAYLQLSSL TSEDTAVYYC NEGTPTGPYY FDYWGQGTTV TVSSGGGGSG |
| | | GGGSGGGGSE NVLTQSPAIM SASPGEKVTI TCSASSSVSY MHWFQQKPGT |
| | | SPKLWIYSTS NLASGVPARF SGSGSGTSYS LTISRMEAED AATYYCQQRS |
| | | SYPLTFGAGT KLELKRAAAG SGGSGFWVLV VVGGVLACYS LLVTVAFIIF |
| | | WVRSKRSRLL HSDYMNMTPR RPGPTRKHYQ PYAPPRDFAA YRSKKVAKKP |
| | | TNKAPHPKQE PQEINFPDDL PGSNTAAPVQ ETLHGCQPVT QEDGKESRIS |
| | | VQERQ |
| 57 | IL2_MFE23_ | MYRMQLLSCI ALSLALVTNS QVKLQQSGAE LVRSGTSVKL SCTASGFNIK |
| | CD28TM_ | DSYMHWLRQG PEQGLEWIGW IDPENGDTEY APKFQGKATF TTDTSSNTAY |
| | CD28_CD40 | LQLSSLTSED TAVYYCNEGT PTGPYYFDYW GQGTTVTVSS GGGGSGGGGS |
| | | GGGGSENVLT QSPAIMSASP GEKVTITCSA SSSVSYMHWF QQKPGTSPKL |
| | | WIYSTSNLAS GVPARFSGSG SGTSYSLTIS RMEAEDAATY YCQQRSSYPL |
| | | TFGAGTKLEL KRAAAGSGGS GFWVLVVVGG VLACYSLLVT VAFIIFWVRS |
| | | KRSRLLHSDY MNMTPRRPGP TRKHYQPYAP PRDFAAYRSK KVAKKPTNKA |
| | | PHPKQEPQEI NFPDDLPGSN TAAPVQETLH GCQPVTQEDG KESRISVQER |
| | | Q |
| 58 | GM-CSF_MFE23_ | MWLQSLLLLG TVACSISQVK LQQSGAELVR SGTSVKLSCT ASGFNIKDSY |
| | CD28TM_ | MHWLRQGPEQ GLEWIGWIDP ENGDTEYAPK FQGKATFTTD TSSNTAYLQL |
| | CD28_CD40 | SSLTSEDTAV YYCNEGTPTG PYYFDYWGQG TTVTVSSGGG GSGGGGSGGG |
| | | GSENVLTQSP AIMSASPGEK VTITCSASSS VSYMHWFQQK PGTSPKLWIY |
| | | STSNLASGVP ARFSGSGSGT SYSLTISRME AEDAATYYCQ QRSSYPLTFG |
| | | AGTKLELKRA AAGSGGSGFW VLVVVGGVLA CYSLLVTVAF IIFWVRSKRS |
| | | [symbol] |
| 59 | hIgGk- | MEAPAQLLFL LLLWLPDTTR QVKLQQSGAE LVRSGTSVKL SCTASGFNIK |
| | VIII_MFE23_ | DSYMHWLRQG PEQGLEWIGW IDPENGDTEY APKFQGKATF TTDTSSNTAY |
| | CD28TM_ | LQLSSLTSED TAVYYCNEGT PTGPYYFDYW GQGTTVTVSS GGGGSGGGGS |
| | CD28_CD40 | GGGGSENVLT QSPAIMSASP GEKVTITCSA SSSVSYMHWF QQKPGTSPKL |
| | | WIYSTSNLAS GVPARFSGSG SGTSYSLTIS RMEAEDAATY YCQQRSSYPL |
| | | TFGAGTKLEL KRAAAGSGGS GFWVLVVVGG VLACYSLLVT VAFIIFWVRS |
| | | KRSRLLHSDY MNMTPRRPGP TRKHYQPYAP PRDFAAYRSK KVAKKPTNKA |
| | | PHPKQEPQEI NFPDDLPGSN TAAPVQETLH GCQPVTQEDG KESRISVQER |
| | | Q |
| 60 | OSM_MFE23 | MGVLLTQRTL LSLVLALLFP SMASMQVQLQ QSGAELVRSG TSVKLSCTAS |
| | (K > Q)_ | GFNIKDSYMH WLRQGPEQGL EWIGWIDPEN GDTEYAPKFQ GKATFTTDTS |
| | spCD28_CD28_ | SNTAYLQLSS LTSEDTAVYY CNEGTPTGPY YFDYWGQGTT VTVSSGGGGS |
| | CD40 | GGGGSGGGGS ENVLTQSPAI MSASPGEKVT ITCSASSSVS YMHWFQQKPG |
| | CTP219 | TSPKLWIYST SNLASGVPAR FSGSGSGTSY SLTISRMEAE DAATYYCQQR |
| | | SSYPLTFGAG TKLELKRAAA GSGGSGILVK QSPMLVAYDN AVNLSCKYSY |
| | | NLFSREFRAS LHKGLDSAVE VCVVYGNYSQ QLQVYSKTGF NCDGKLGNES |
| | | VTFYLQNLYV NQTDIYFCKI EVMYPPPYLD NEKSNGTIIH VKGKHLCPSP |
| | | LFPGPSKPFW VLVVVGGVLA CYSLLVTVAF IIFWVRSKRS RLLHSDYMNM |
| | | TPRRPGPTRK HYQPYAPPRD FAAYRSKKVA KKPTNKAPHP KQEPQEINFP |
| | | DDLPGSNTAA PVQETLHGCQ PVTQEDGKES RISVQERQ |
| 61 | CD8a_MFE23 | MALPVTALLL PLALLLHAAR PQVQLQQSGA ELVRSGTSVK LSCTASGFNI |
| | (K > Q)_ | KDSYMHWLRQ GPEQGLEWIG WIDPENGDTE YAPKFQGKAT FTTDTSSNTA |
| | spCD28_CD28_ | YLQLSSLTSE DTAVYYCNEG TPTGPYYFDY WGQGTTVTVS SGGGGSGGGG |
| | CD40 | SGGGGSENVL TQSPAIMSAS PGEKVTITCS ASSSVSYMHW FQQKPGTSPK |
| | | LWIYSTSNLA SGVPARFSGS GSGTSYSLTI SRMEAEDAAT YYCQQRSSYP |
| | | LTFGAGTKLE LKRAAAGSGG SGILVKQSPM LVAYDNAVNL SCKYSYNLFS |
| | | REFRASLHKG LDSAVEVCVV YGNYSQQLQV YSKTGFNCDG KLGNESVTFY |
| | | LQNLYVNQTD IYFCKIEVMY PPPYLDNEKS NGTIIHVKGK HLCPSPLFPG |
| | | PSKPFWVLVV VGGVLACYSL LVTVAFIIFW VRSKRSRLLH SDYMNMTPRR |
| | | PGPTRKHYQP YAPPRDFAAY RSKKVAKKPT NKAPHPKQEP QEINFPDDLP |
| | | GSNTAAPVQE TLHGCQPVTQ EDGKESRISV QERQ |
| 62 | CD2_MFE23 | MSFPCKFVAS FLLIFNVSSK GAVSQVQLQQ SGAELVRSGT SVKLSCTASG |
| | (K > Q)_ | FNIKDSYMHW LRQGPEQGLE WIGWIDPENG DTEYAPKFQG KATFTTDTSS |
| | spCD28_ | NTAYLQLSSL TSEDTAVYYC NEGTPTGPYY FDYWGQGTTV TVSSGGGGSG |
| | CD28_CD40 | GGGSGGGGSE NVLTQSPAIM SASPGEKVTI TCSASSSVSY MHWFQQKPGT |
| | | SPKLWIYSTS NLASGVPARF SGSGSGTSYS LTISRMEAED AATYYCQQRS |
| | | SYPLTFGAGT KLELKRAAAG SGGSGILVKQ SPMLVAYDNA VNLSCKYSYN |
| | | LFSREFRASL HKGLDSAVEV CVVYGNYSQQ LQVYSKTGFN CDGKLGNESV |
| | | TFYLQNLYVN QTDIYFCKIE VMYPPPYLDN EKSNGTIIHV KGKHLCPSPL |
| | | FPGPSKPFWV LVVVGGVLAC YSLLVTVAFI IFWVRSKRSR LLHSDYMNMT |
| | | PRRPGPTRKH YQPYAPPRDF AAYRSKKVAK KPTNKAPHPK QEPQEINFPD |
| | | DLPGSNTAAP VQETLHGCQP VTQEDGKESR ISVQERQ |
| 63 | IL2_MFE23 | MYRMQLLSCI ALSLALVTNS QVQLQQSGAE LVRSGTSVKL SCTASGFNIK |
| | (K > Q)_ | DSYMHWLRQG PEQGLEWIGW IDPENGDTEY APKFQGKATF TTDTSSNTAY |
| | spCD28_ | LQLSSLTSED TAVYYCNEGT PTGPYYFDYW GQGTTVTVSS GGGGSGGGGS |
| | CD28_CD40 | GGGGSENVLT QSPAIMSASP GEKVTITCSA SSSVSYMHWF QQKPGTSPKL |
| | | WIYSTSNLAS GVPARFSGSG SGTSYSLTIS RMEAEDAATY YCQQRSSYPL |
| | | TFGAGTKLEL KRAAAGSGGS GILVKQSPML VAYDNAVNLS CKYSYNLFSR |
| | | EFRASLHKGL DSAVEVCVVY GNYSQQLQVY SKTGFNCDGK LGNESVTFYL |
| | | QNLYVNQTDI YFCKIEVMYP PPYLDNEKSN GTIIHVKGKH LCPSPLFPGP |
| | | SKPFWVLVVV GGVLACYSLL VTVAFIIFWV RSKRSRLLHS DYMNMTPRRP |
| | | GPTRKHYQPY APPRDFAAYR SKKVAKKPTN KAPHPKQEPQ EINFPDDLPG |
| | | SNTAAPVQET LHGCQPVTQE DGKESRISVQ ERQ |
| 64 | GM-CSF_ | MWLQSLLLLG TVACSISQVQ LQQSGAELVR SGTSVKLSCT ASGFNIKDSY |
| | MFE23(K > Q)_ | MHWLRQGPEQ GLEWIGWIDP ENGDTEYAPK FQGKATFTTD TSSNTAYLQL |
| | spCD28_ | SSLTSEDTAV YYCNEGTPTG PYYFDYWGQG TTVTVSSGGG GSGGGGSGGG |
| | CD28_CD40 | GSENVLTQSP AIMSASPGEK VTITCSASSS VSYMHWFQQK PGTSPKLWIY |
| | | STSNLASGVP ARFSGSGSGT SYSLTISRME AEDAATYYCQ QRSSYPLTFG |
| | | AGTKLELKRA AAGSGGSGIL VKQSPMLVAY DNAVNLSCKY SYNLFSREFR |
| | | ASLHKGLDSA VEVCVVYGNY SQQLQVYSKT GFNCDGKLGN ESVTFYLQNL |
| | | YVNQTDIYFC KIEVMYPPPY LDNEKSNGTI IHVKGKHLCP SPLFPGPSKP |
| | | FWVLVVVGGV LACYSLLVTV AFIIFWVRSK RSRLLHSDYM NMTPRRPGPT |
| | | RKHYQPYAPP RDFAAYRSKK VAKKPTNKAP HPKQEPQEIN FPDDLPGSNT |
| | | AAPVQETLHG CQPVTQEDGK ESRISVQERQ |
| 65 | hIgGk-VIII_ | MEAPAQLLFL LLLWLPDTTR QVQLQQSGAE LVRSGTSVKL SCTASGFNIK |
| | MFE23(K > Q)_ | DSYMHWLRQG PEQGLEWIGW IDPENGDTEY APKFQGKATF TTDTSSNTAY |
| | spCD28_ | LQLSSLTSED TAVYYCNEGT PTGPYYFDYW GQGTTVTVSS GGGGSGGGGS |
| | CD28_CD40 | GGGGSENVLT QSPAIMSASP GEKVTITCSA SSSVSYMHWF QQKPGTSPKL |
| | | WIYSTSNLAS GVPARFSGSG SGTSYSLTIS RMEAEDAATY YCQQRSSYPL |
| | | TFGAGTKLEL KRAAAGSGGS GILVKQSPML VAYDNAVNLS CKYSYNLFSR |
| | | EFRASLHKGL DSAVEVCVVY GNYSQQLQVY SKTGFNCDGK LGNESVTFYL |
| | | QNLYVNQTDI YFCKIEVMYP PPYLDNEKSN GTIIHVKGKH LCPSPLFPGP |
| | | SKPFWVLVVV GGVLACYSLL VTVAFIIFWV RSKRSRLLHS DYMNMTPRRP |
| | | GPTRKHYQPY APPRDFAAYR SKKVAKKPTN KAPHPKQEPQ EINFPDDLPG |
| | | SNTAAPVQET LHGCQPVTQE DGKESRISVQ ERQ |
| 66 | OSM_MFE23 | MGVLLTQRTL LSLVLALLFP SMASMQVOLQ QSGAELVRSG TSVKLSCTAS |
| | (K > Q)_ | GFNIKDSYMH WLRQGPEQGL EWIGWIDPEN GDTEYAPKFQ GKATFTTDTS |
| | spCD8_CD28_ | SNTAYLQLSS LTSEDTAVYY CNEGTPTGPY YFDYWGQGTT VTVSSGGGGS |
| | CD40 | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGFVPV FLPAKPTTTP APRPPTPAPT |
| | | IASQPLSLRP EACRPAAGGA VHTRGLDFAC DIYIWAPLAG TCGVLLLSLV |
| | | ITLYCNHRNR SKRSRLLHSD YMNMTPRRPG PTRKHYQPYA PPRDFAAYRS |
| | | KKVAKKPTNK APHPKQEPQE INFPDDLPGS NTAAPVQETL HGCQPVTQED |
| | | GKESRISVQE RQ |
| 67 | CD8a_ | MALPVTALLL PLALLLHAAR PQVQLQQSGA ELVRSGTSVK LSCTASGFNI |
| | MFE23(K > Q)_ | KDSYMHWLRQ GPEQGLEWIG WIDPENGDTE YAPKFQGKAT FTTDTSSNTA |
| | spCD8_ | YLQLSSLTSE DTAVYYCNEG TPTGPYYFDY WGQGTTVTVS SGGGGSGGGG |
| | CD28_CD40 | SGGGGSENVL TQSPAIMSAS PGEKVTITCS ASSSVSYMHW FQQKPGTSPK |
| | | LWIYSTSNLA SGVPARFSGS GSGTSYSLTI SRMEAEDAAT YYCQQRSSYP |
| | | LTFGAGTKLE LKRAAAGSGG SGFVPVFLPA KPTTTPAPRP PTPAPTIASQ |
| | | PLSLRPEACR PAAGGAVHTR GLDFACDIYI WAPLAGTCGV LLLSLVITLY |
| | | CNHRNRSKRS RLLHSDYMNM TPRRPGPTRK HYQPYAPPRD FAAYRSKKVA |
| | | KKPTNKAPHP KQEPQEINFP DDLPGSNTAA PVQETLHGCQ PVTQEDGKES |
| | | RISVQERQ |
| 68 | CD2_MFE23 | MSFPCKFVAS FLLIFNVSSK GAVSQVQLQQ SGAELVRSGT SVKLSCTASG |
| | (K > Q)_ | FNIKDSYMHW LRQGPEQGLE WIGWIDPENG DTEYAPKFQG KATFTTDTSS |
| | spCD8_ | NTAYLQLSSL TSEDTAVYYC NEGTPTGPYY FDYWGQGTTV TVSSGGGGSG |
| | CD28_CD40 | GGGSGGGGSE NVLTQSPAIM SASPGEKVTI TCSASSSVSY MHWFQQKPGT |
| | | SPKLWIYSTS NLASGVPARF SGSGSGTSYS LTISRMEAED AATYYCQQRS |
| | | SYPLTFGAGT KLELKRAAAG SGGSGFVPVF LPAKPTTTPA PRPPTPAPTI |
| | | ASQPLSLRPE ACRPAAGGAV HTRGLDFACD IYIWAPLAGT CGVLLLSLVI |
| | | TLYCNHRNRS KRSRLLHSDY MNMTPRRPGP TRKHYQPYAP PRDFAAYRSK |
| | | KVAKKPTNKA PHPKQEPQEI NFPDDLPGSN TAAPVQETLH GCQPVTQEDG |
| | | KESRISVQER Q |
| 69 | IL2_MFE23 | MYRMQLLSCI ALSLALVTNS QVQLQQSGAE LVRSGTSVKL SCTASGFNIK |
| | (K > Q)_ | DSYMHWLRQG PEQGLEWIGW IDPENGDTEY APKFQGKATF TTDTSSNTAY |
| | spCD8_ | LQLSSLTSED TAVYYCNEGT PTGPYYFDYW GQGTTVTVSS GGGGSGGGGS |
| | CD28_CD40 | GGGGSENVLT QSPAIMSASP GEKVTITCSA SSSVSYMHWF QQKPGTSPKL |
| | | WIYSTSNLAS GVPARFSGSG SGTSYSLTIS RMEAEDAATY YCQQRSSYPL |
| | | TFGAGTKLEL KRAAAGSGGS GFVPVFLPAK PTTTPAPRPP TPAPTIASQP |
| | | LSLRPEACRP AAGGAVHTRG LDFACDIYIW APLAGTCGVL LLSLVITLYC |
| | | NHRNRSKRSR LLHSDYMNMT PRRPGPTRKH YQPYAPPRDF AAYRSKKVAK |
| | | KPTNKAPHPK QEPQEINFPD DLPGSNTAAP VQETLHGCQP VTQEDGKESR |
| | | ISVQERQ |
| 70 | GM-CSF_ | MWLQSLLLLG TVACSISQVQ LQQSGAELVR SGTSVKLSCT ASGFNIKDSY |
| | MFE23(K > Q)_ | MHWLRQGPEQ GLEWIGWIDP ENGDTEYAPK FQGKATFTTD TSSNTAYLQL |
| | spCD8_ | SSLTSEDTAV YYCNEGTPTG PYYFDYWGQG TTVTVSSGGG GSGGGGSGGG |
| | CD28_CD40 | GSENVLTQSP AIMSASPGEK VTITCSASSS VSYMHWFQQK PGTSPKLWIY |
| | | STSNLASGVP ARFSGSGSGT SYSLTISRME AEDAATYYCQ QRSSYPLTFG |
| | | AGTKLELKRA AAGSGGSGFV PVFLPAKPTT TPAPRPPTPA PTIASQPLSL |
| | | RPEACRPAAG GAVHTRGLDF ACDIYIWAPL AGTCGVLLLS LVITLYCNHR |
| | | NRSKRSRLLH SDYMNMTPRR PGPTRKHYQP YAPPRDFAAY RSKKVAKKPT |
| | | NKAPHPKQEP QEINFPDDLP GSNTAAPVQE TLHGCQPVTQ EDGKESRISV |
| | | QERQ |
| 71 | hIgGk-VIII_ | MEAPAQLLFL LLLWLPDTTR QVQLQQSGAE LVRSGTSVKL SCTASGFNIK |
| | MFE23(K > Q)_ | DSYMHWLRQG PEQGLEWIGW IDPENGDTEY APKFQGKATF TTDTSSNTAY |
| | spCD8_ | LQLSSLTSED TAVYYCNEGT PTGPYYFDYW GQGTTVTVSS GGGGSGGGGS |
| | CD28_CD40 | GGGGSENVLT QSPAIMSASP GEKVTITCSA SSSVSYMHWF QQKPGTSPKL |
| | | WIYSTSNLAS GVPARFSGSG SGTSYSLTIS RMEAEDAATY YCQQRSSYPL |
| | | TFGAGTKLEL KRAAAGSGGS GFVPVFLPAK PTTTPAPRPP TPAPTIASQP |
| | | LSLRPEACRP AAGGAVHTRG LDFACDIYIW APLAGTCGVL LLSLVITLYC |
| | | NHRNRSKRSR LLHSDYMNMT PRRPGPTRKH YQPYAPPRDF AAYRSKKVAK |
| | | KPTNKAPHPK QEPQEINFPD DLPGSNTAAP VQETLHGCQP VTQEDGKESR |
| | | ISVQERQ |
| 72 | OSM_MFE23 | MGVLLTQRTL LSLVLALLFP SMASMQVQLQ QSGAELVRSG TSVKLSCTAS |
| | (K > Q)_ | GFNIKDSYMH WLRQGPEQGL EWIGWIDPEN GDTEYAPKFQ GKATFTTDTS |
| | CD28TM_ | SNTAYLQLSS LTSEDTAVYY CNEGTPTGPY YFDYWGQGTT VTVSSGGGGS |
| | CD28_CD40 | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGFWVL VVVGGVLACY SLLVTVAFII |
| | | FWVRSKRSRL LHSDYMNMTP RRPGPTRKHY QPYAPPRDFA AYRSKKVAKK |
| | | PTNKAPHPKQ EPQEINFPDD LPGSNTAAPV QETLHGCQPV TQEDGKESRI |
| | | SVQERQ |
| 73 | CD8a_ | MALPVTALLL PLALLLHAAR PQVQLQQSGA ELVRSGTSVK LSCTASGFNI |
| | MFE23(K > Q)_ | KDSYMHWLRQ GPEQGLEWIG WIDPENGDTE YAPKFQGKAT FTTDTSSNTA |
| | CD28TM_ | YLQLSSLTSE DTAVYYCNEG TPTGPYYFDY WGQGTTVTVS SGGGGSGGGG |
| | CD28_CD40 | SGGGGSENVL TQSPAIMSAS PGEKVTITCS ASSSVSYMHW FQQKPGTSPK |
| | | LWIYSTSNLA SGVPARFSGS GSGTSYSLTI SRMEAEDAAT YYCQQRSSYP |
| | | LTFGAGTKLE LKRAAAGSGG SGFWVLVVVG GVLACYSLLV TVAFIIFWVR |
| | | SKRSRLLHSD YMNMTPRRPG PTRKHYQPYA PPRDFAAYRS KKVAKKPTNK |
| | | APHPKQEPQE INFPDDLPGS NTAAPVQETL HGCQPVTQED GKESRISVQE |
| | | RQ |
| 74 | CD2_MFE23 | MSFPCKFVAS FLLIFNVSSK GAVSQVQLQQ SGAELVRSGT SVKLSCTASG |
| | (K > Q)_ | FNIKDSYMHW LRQGPEQGLE WIGWIDPENG DTEYAPKFQG KATFTTDTSS |
| | CD28TM_ | NTAYLQLSSL TSEDTAVYYC NEGTPTGPYY FDYWGQGTTV TVSSGGGGSG |
| | CD28_CD40 | GGGSGGGGSE NVLTQSPAIM SASPGEKVTI TCSASSSVSY MHWFQQKPGT |
| | | SPKLWIYSTS NLASGVPARF SGSGSGTSYS LTISRMEAED AATYYCQQRS |
| | | SYPLTFGAGT KLELKRAAAG SGGSGFWVLV VVGGVLACYS LLVTVAFIIF |
| | | WVRSKRSRLL HSDYMNMTPR RPGPTRKHYQ PYAPPRDFAA YRSKKVAKKP |
| | | TNKAPHPKQE PQEINFPDDL PGSNTAAPVQ ETLHGCQPVT QEDGKESRIS |
| | | VQERQ |
| 75 | IL2_MFE23 | MYRMQLLSCI ALSLALVTNS QVQLQQSGAE LVRSGTSVKL SCTASGFNIK |
| | (K > Q)_ | DSYMHWLRQG PEQGLEWIGW IDPENGDTEY APKFQGKATF TTDTSSNTAY |
| | CD28TM_ | LQLSSLTSED TAVYYCNEGT PTGPYYFDYW GQGTTVTVSS GGGGSGGGGS |
| | CD28_CD40 | GGGGSENVLT QSPAIMSASP GEKVTITCSA SSSVSYMHWF QQKPGTSPKL |
| | | WIYSTSNLAS GVPARFSGSG SGTSYSLTIS RMEAEDAATY YCQQRSSYPL |
| | | TFGAGTKLEL KRAAAGSGGS GFWVLVVVGG VLACYSLLVT VAFIIFWVRS |
| | | KRSRLLHSDY MNMTPRRPGP TRKHYQPYAP PRDFAAYRSK KVAKKPTNKA |
| | | PHPKQEPQEI NFPDDLPGSN TAAPVQETLH GCQPVTQEDG KESRISVQER |
| | | Q |
| 76 | GM-CSF_ | MWLQSLLLLG TVACSISQVQ LQQSGAELVR SGTSVKLSCT ASGFNIKDSY |
| | MFE23(K > Q)_ | MHWLRQGPEQ GLEWIGWIDP ENGDTEYAPK FQGKATFTTD TSSNTAYLQL |
| | CD28TM_ | SSLTSEDTAV YYCNEGTPTG PYYFDYWGQG TTVTVSSGGG GSGGGGSGGG |
| | CD28_CD40 | GSENVLTQSP AIMSASPGEK VTITCSASSS VSYMHWFQQK PGTSPKLWIY |
| | | STSNLASGVP ARFSGSGSGT SYSLTISRME AEDAATYYCQ QRSSYPLTFG |
| | | AGTKLELKRA AAGSGGSGFW VLVVVGGVLA CYSLLVTVAF IIFWVRSKRS |
| | | RLLHSDYMNM TPRRPGPTRK HYQPYAPPRD FAAYRSKKVA KKPTNKAPHP |
| | | KQEPQEINFP DDLPGSNTAA PVQETLHGCQ PVTQEDGKES RISVQERQ |
| 77 | hIgGk-VIII_ | MEAPAQLLFL LLLWLPDTTR QVQLQQSGAE LVRSGTSVKL SCTASGFNIK |
| | MFE23(K > Q)_ | DSYMHWLRQG PEQGLEWIGW IDPENGDTEY APKFQGKATF TTDTSSNTAY |
| | CD28TM_ | LQLSSLTSED TAVYYCNEGT PTGPYYFDYW GQGTTVTVSS GGGGSGGGGS |
| | CD28_CD40 | GGGGSENVLT QSPAIMSASP GEKVTITCSA SSSVSYMHWF QQKPGTSPKL |
| | | WIYSTSNLAS GVPARFSGSG SGTSYSLTIS RMEAEDAATY YCQQRSSYPL |
| | | TFGAGTKLEL KRAAAGSGGS GFWVLVVVGG VLACYSLLVT VAFIIFWVRS |
| | | KRSRLLHSDY MNMTPRRPGP TRKHYQPYAP PRDFAAYRSK KVAKKPTNKA |
| | | PHPKQEPQEI NFPDDLPGSN TAAPVQETLH GCQPVTQEDG KESRISVQER |
| | | Q |
| 78 | OSM_HuMFE23_ | MGVLLTQRTL LSLVLALLFP SMASMQVKLE QSGAEVVKPG ASVKLSCKAS |
| | spCD28_ | GFNIKDSYMH WLRQGPGQRL EWIGWIDPEN GDTEYAPKFQ GKATFTTDTS |
| | CD28_CD40 | ANTAYLGLSS LRPEDTAVYY CNEGTPTGPY YFDYWGQGTL VTVSSGGGGS |
| | CTP220 | GGGGSGGGGS ENVLTQSPSS MSASVGDRVN IACSASSSVS YMHWFQQKPG |
| | | KSPKLWIYST SNLASGVPSR FSGSGSGTDY SLTISSMQPE DAATYYCQQR |
| | | SSYPLTFGGG TKLEIKAAAG SGGSGILVKQ SPMLVAYDNA VNLSCKYSYN |
| | | LFSREFRASL HKGLDSAVEV CVVYGNYSQQ LQVYSKTGFN CDGKLGNESV |
| | | TFYLQNLYVN QTDIYFCKIE VMYPPPYLDN EKSNGTIIHV KGKHLCPSPL |
| | | FPGPSKPFWV LVVVGGVLAC YSLLVTVAFI IFWVRSKRSR LLHSDYMNMT |
| | | PRRPGPTRKH YQPYAPPRDF AAYRSKKVAK KPTNKAPHPK QEPQEINFPD |
| | | DLPGSNTAAP VQETLHGCQP VTQEDGKESR ISVQERQ |
| 79 | CD8a_HuMFE23_ | MALPVTALLL PLALLLHAAR PQVKLEQSGA EVVKPGASVK LSCKASGFNI |
| | spCD28_ | KDSYMHWLRQ GPGQRLEWIG WIDPENGDTE YAPKFQGKAT FTTDTSANTA |
| | CD28_CD40 | YLGLSSLRPE DTAVYYCNEG TPTGPYYFDY WGQGTLVTVS SGGGGSGGGG |
| | | SGGGGSENVL TQSPSSMSAS VGDRVNIACS ASSSVSYMHW FQQKPGKSPK |
| | | LWIYSTSNLA SGVPSRFSGS GSGTDYSLTI SSMQPEDAAT YYCQQRSSYP |
| | | LTFGGGTKLE IKAAAGSGGS GILVKQSPML VAYDNAVNLS CKYSYNLFSR |
| | | EFRASLHKGL DSAVEVCVVY GNYSQQLQVY SKTGFNCDGK LGNESVTFYL |
| | | QNLYVNQTDI YFCKIEVMYP PPYLDNEKSN GTIIHVKGKH LCPSPLFPGP |
| | | SKPFWVLVVV GGVLACYSLL VTVAFIIFWV RSKRSRLLHS DYMNMTPRRP |
| | | GPTRKHYQPY APPRDFAAYR SKKVAKKPTN KAPHPKQEPQ EINFPDDLPG |
| | | SNTAAPVQET LHGCQPVTQE DGKESRISVQ ERQ |
| 80 | CD2_HuMFE23_ | MSFPCKFVAS FLLIFNVSSK GAVSQVKLEQ SGAEVVKPGA SVKLSCKASG |
| | spCD28_ | FNIKDSYMHW LRQGPGQRLE WIGWIDPENG DTEYAPKFQG KATFTTDTSA |
| | CD28_CD40 | NTAYLGLSSL RPEDTAVYYC NEGTPTGPYY FDYWGQGTLV TVSSGGGGSG |
| | | GGGSGGGGSE NVLTQSPSSM SASVGDRVNI ACSASSSVSY MHWFQQKPGK |
| | | SPKLWIYSTS NLASGVPSRF SGSGSGTDYS LTISSMQPED AATYYCQQRS |
| | | SYPLTFGGGT KLEIKAAAGS GGSGILVKQS PMLVAYDNAV NLSCKYSYNL |
| | | FSREFRASLH KGLDSAVEVC VVYGNYSQQL QVYSKTGFNC DGKLGNESVT |
| | | FYLQNLYVNQ TDIYFCKIEV MYPPPYLDNE KSNGTIIHVK GKHLCPSPLF |
| | | PGPSKPFWVL VVVGGVLACY SLLVTVAFII FWVRSKRSRL LHSDYMNMTP |
| | | RRPGPTRKHY QPYAPPRDFA AYRSKKVAKK PTNKAPHPKQ EPQEINFPDD |
| | | LPGSNTAAPV QETLHGCQPV TQEDGKESRI SVQERQ |
| 81 | IL2_HuMFE23_ | MYRMQLLSCI ALSLALVTNS QVKLEQSGAE VVKPGASVKL SCKASGFNIK |
| | spCD28_ | DSYMHWLRQG PGQRLEWIGW IDPENGDTEY APKFQGKATF TTDTSANTAY |
| | CD28_CD40 | LGLSSLRPED TAVYYCNEGT PTGPYYFDYW GQGTLVTVSS GGGGSGGGGS |
| | | GGGGSENVLT QSPSSMSASV GDRVNIACSA SSSVSYMHWF QQKPGKSPKL |
| | | WIYSTSNLAS GVPSRFSGSG SGTDYSLTIS SMQPEDAATY YCQQRSSYPL |
| | | TFGGGTKLEI KAAAGSGGSG ILVKQSPMLV AYDNAVNLSC KYSYNLFSRE |
| | | FRASLHKGLD SAVEVCVVYG NYSQQLQVYS KTGFNCDGKL GNESVTFYLQ |
| | | NLYVNQTDIY FCKIEVMYPP PYLDNEKSNG TIIHVKGKHL CPSPLFPGPS |
| | | KPFWVLVVVG GVLACYSLLV TVAFIIFWVR SKRSRLLHSD YMNMTPRRPG |
| | | PTRKHYQPYA PPRDFAAYRS KKVAKKPTNK APHPKQEPQE INFPDDLPGS |
| | | NTAAPVQETL HGCQPVTQED GKESRISVQE RQ |
| 82 | GM-CSF_ | MWLQSLLLLG TVACSISQVK LEQSGAEVVK PGASVKLSCK ASGFNIKDSY |
| | HuMFE23_ | MHWLRQGPGQ RLEWIGWIDP ENGDTEYAPK FQGKATFTTD TSANTAYLGL |
| | spCD28_ | SSLRPEDTAV YYCNEGTPTG PYYFDYWGQG TLVTVSSGGG GSGGGGSGGG |
| | CD28_CD40 | GSENVLTQSP SSMSASVGDR VNIACSASSS VSYMHWFQQK PGKSPKLWIY |
| | | STSNLASGVP SRFSGSGSGT DYSLTISSMQ PEDAATYYCQ QRSSYPLTFG |
| | | GGTKLEIKAA AGSGGSGILV KQSPMLVAYD NAVNLSCKYS YNLFSREFRA |
| | | SLHKGLDSAV EVCVVYGNYS QQLQVYSKTG FNCDGKLGNE SVTFYLQNLY |
| | | VNQTDIYFCK IEVMYPPPYL DNEKSNGTII HVKGKHLCPS PLFPGPSKPF |
| | | WVLVVVGGVL ACYSLLVTVA FIIFWVRSKR SRLLHSDYMN MTPRRPGPTR |
| | | KHYQPYAPPR DFAAYRSKKV AKKPTNKAPH PKQEPQEINF PDDLPGSNTA |
| | | APVQETLHGC QPVTQEDGKE SRISVQERQ |
| 83 | hIgGk-VIII_ | MEAPAQLLFL LLLWLPDTTR QVKLEQSGAE VVKPGASVKL SCKASGFNIK |
| | HuMFE23_ | DSYMHWLRQG PGQRLEWIGW IDPENGDTEY APKFQGKATF TTDTSANTAY |
| | spCD28_ | LGLSSLRPED TAVYYCNEGT PTGPYYFDYW GQGTLVTVSS GGGGSGGGGS |
| | CD28_CD40 | GGGGSENVLT QSPSSMSASV GDRVNIACSA SSSVSYMHWF QQKPGKSPKL |
| | | WIYSTSNLAS GVPSRFSGSG SGTDYSLTIS SMQPEDAATY YCQQRSSYPL |
| | | TFGGGTKLEI KAAAGSGGSG ILVKQSPMLV AYDNAVNLSC KYSYNLFSRE |
| | | FRASLHKGLD SAVEVCVVYG NYSQQLQVYS KTGFNCDGKL GNESVTFYLQ |
| | | NLYVNQTDIY FCKIEVMYPP PYLDNEKSNG TIIHVKGKHL CPSPLFPGPS |
| | | KPFWVLVVVG GVLACYSLLV TVAFIIFWVR SKRSRLLHSD YMNMTPRRPG |
| | | PTRKHYQPYA PPRDFAAYRS KKVAKKPTNK APHPKQEPQE INFPDDLPGS |
| | | NTAAPVQETL HGCQPVTQED GKESRISVQE RQ |
| 84 | OSM_HuMFE23_ | MGVLLTQRTL LSLVLALLFP SMASMQVKLE QSGAEVVKPG ASVKLSCKAS |
| | spCD8_ | GFNIKDSYMH WLRQGPGQRL EWIGWIDPEN GDTEYAPKFQ GKATFTTDTS |
| | CD28_CD40 | ANTAYLGLSS LRPEDTAVYY CNEGTPTGPY YFDYWGQGTL VTVSSGGGGS |
| | CTP232 | GGGGSGGGGS ENVLTQSPSS MSASVGDRVN IACSASSSVS YMHWFQQKPG |
| | | KSPKLWIYST SNLASGVPSR FSGSGSGTDY SLTISSMQPE DAATYYCQQR |
| | | SSYPLTFGGG TKLEIKAAAG SGGSGFVPVF LPAKPTTTPA PRPPTPAPTI |
| | | ASQPLSLRPE ACRPAAGGAV HTRGLDFACD IYIWAPLAGT CGVLLLSLVI |
| | | TLYCNHRNRS KRSRLLHSDY MNMTPRRPGP TRKHYQPYAP PRDFAAYRSK |
| | | KVAKKPTNKA PHPKQEPQEI NFPDDLPGSN TAAPVQETLH GCQPVTQEDG |
| | | KESRISVQER Q |
| 85 | CD8a_HuMFE23_ | MALPVTALLL PLALLLHAAR PQVKLEQSGA EVVKPGASVK LSCKASGFNI |
| | spCD8_ | KDSYMHWLRQ GPGQRLEWIG WIDPENGDTE YAPKFQGKAT FTTDTSANTA |
| | CD28_CD40 | YLGLSSLRPE DTAVYYCNEG TPTGPYYFDY WGQGTLVTVS SGGGGSGGGG |
| | | SGGGGSENVL TQSPSSMSAS VGDRVNIACS ASSSVSYMHW FQQKPGKSPK |
| | | LWIYSTSNLA SGVPSRFSGS GSGTDYSLTI SSMQPEDAAT YYCQQRSSYP |
| | | LTFGGGTKLE IKAAAGSGGS GFVPVFLPAK PTTTPAPRPP TPAPTIASQP |
| | | LSLRPEACRP AAGGAVHTRG LDFACDIYIW APLAGTCGVL LLSLVITLYC |
| | | NHRNRSKRSR LLHSDYMNMT PRRPGPTRKH YQPYAPPRDF AAYRSKKVAK |
| | | KPTNKAPHPK QEPQEINFPD DLPGSNTAAP VQETLHGCQP VTQEDGKESR |
| | | ISVQERQ |
| 86 | CD2_HuMFE23_ | MSFPCKFVAS FLLIFNVSSK GAVSQVKLEQ SGAEVVKPGA SVKLSCKASG |
| | spCD8_ | FNIKDSYMHW LRQGPGQRLE WIGWIDPENG DTEYAPKFQG KATFTTDTSA |
| | CD28_CD40 | NTAYLGLSSL RPEDTAVYYC NEGTPTGPYY FDYWGQGTLV TVSSGGGGSG |
| | | GGGSGGGGSE NVLTQSPSSM SASVGDRVNI ACSASSSVSY MHWFQQKPGK |
| | | SPKLWIYSTS NLASGVPSRF SGSGSGTDYS LTISSMQPED AATYYCQQRS |
| | | SYPLTFGGGT KLEIKAAAGS GGSGFVPVFL PAKPTTTPAP RPPTPAPTIA |
| | | SQPLSLRPEA CRPAAGGAVH TRGLDFACDI YIWAPLAGTC GVLLLSLVIT |
| | | LYCNHRNRSK RSRLLHSDYM NMTPRRPGPT RKHYQPYAPP RDFAAYRSKK |
| | | VAKKPTNKAP HPKQEPQEIN FPDDLPGSNT AAPVQETLHG CQPVTQEDGK |
| | | ESRISVQERQ |
| 87 | IL2_HuMFE23_ | MYRMQLLSCI ALSLALVTNS QVKLEQSGAE VVKPGASVKL SCKASGFNIK |
| | spCD8_ | DSYMHWLRQG PGQRLEWIGW IDPENGDTEY APKFQGKATF TTDTSANTAY |
| | CD28_CD40 | LGLSSLRPED TAVYYCNEGT PTGPYYFDYW GQGTLVTVSS GGGGSGGGGS |
| | | GGGGSENVLT QSPSSMSASV GDRVNIACSA SSSVSYMHWF QQKPGKSPKL |
| | | WIYSTSNLAS GVPSRFSGSG SGTDYSLTIS SMQPEDAATY YCQQRSSYPL |
| | | TFGGGTKLEI KAAAGSGGSG FVPVFLPAKP TTTPAPRPPT PAPTIASQPL |
| | | SLRPEACRPA AGGAVHTRGL DFACDIYIWA PLAGTCGVLL LSLVITLYCN |
| | | HRNRSKRSRL LHSDYMNMTP RRPGPTRKHY QPYAPPRDFA AYRSKKVAKK |
| | | PTNKAPHPKQ EPQEINFPDD LPGSNTAAPV QETLHGCQPV TQEDGKESRI |
| | | SVQERQ |
| 88 | GM-CSF_ | MWLQSLLLLG TVACSISQVK LEQSGAEVVK PGASVKLSCK ASGFNIKDSY |
| | HuMFE23_ | MHWLRQGPGQ RLEWIGWIDP ENGDTEYAPK FQGKATFTTD TSANTAYLGL |
| | spCD8_ | SSLRPEDTAV YYCNEGTPTG PYYFDYWGQG TLVTVSSGGG GSGGGGSGGG |
| | CD28_CD40 | GSENVLTQSP SSMSASVGDR VNIACSASSS VSYMHWFQQK PGKSPKLWIY |
| | | STSNLASGVP SRFSGSGSGT DYSLTISSMQ PEDAATYYCQ QRSSYPLTFG |
| | | GGTKLEIKAA AGSGGSGFVP VFLPAKPTTT PAPRPPTPAP TIASQPLSLR |
| | | PEACRPAAGG AVHTRGLDFA CDIYIWAPLA GTCGVLLLSL VITLYCNHRN |
| | | RSKRSRLLHS DYMNMTPRRP GPTRKHYQPY APPRDFAAYR SKKVAKKPTN |
| | | KAPHPKQEPQ EINFPDDLPG SNTAAPVQET LHGCQPVTQE DGKESRISVQ |
| | | ERQ |
| 89 | hIgGk-VIII_ | MEAPAQLLFL LLLWLPDTTR QVKLEQSGAE VVKPGASVKL SCKASGFNIK |
| | HuMFE23_ | DSYMHWLRQG PGQRLEWIGW IDPENGDTEY APKFQGKATF TTDTSANTAY |
| | spCD8_ | LGLSSLRPED TAVYYCNEGT PTGPYYFDYW GQGTLVTVSS GGGGSGGGGS |
| | CD28_CD40 | GGGGSENVLT QSPSSMSASV GDRVNIACSA SSSVSYMHWF QQKPGKSPKL |
| | | WIYSTSNLAS GVPSRFSGSG SGTDYSLTIS SMQPEDAATY YCQQRSSYPL |
| | | TFGGGTKLEI KAAAGSGGSG FVPVFLPAKP TTTPAPRPPT PAPTIASQPL |
| | | SLRPEACRPA AGGAVHTRGL DFACDIYIWA PLAGTCGVLL LSLVITLYCN |
| | | HRNRSKRSRL LHSDYMNMTP RRPGPTRKHY QPYAPPRDFA AYRSKKVAKK |
| | | PTNKAPHPKQ EPQEINFPDD LPGSNTAAPV QETLHGCQPV TQEDGKESRI |
| | | SVQERQ |
| 90 | OSM_HuMFE23_ | MGVLLTQRTL LSLVLALLFP SMASMQVKLE QSGAEVVKPG ASVKLSCKAS |
| | CD28TM_ | GFNIKDSYMH WLRQGPGQRL EWIGWIDPEN GDTEYAPKFQ GKATFTTDTS |
| | CD28_CD40 | ANTAYLGLSS LRPEDTAVYY CNEGTPTGPY YFDYWGQGTL VTVSSGGGGS |
| | | GGGGSGGGGS ENVLTQSPSS MSASVGDRVN IACSASSSVS YMHWFQQKPG |
| | | KSPKLWIYST SNLASGVPSR FSGSGSGTDY SLTISSMQPE DAATYYCQQR |
| | | SSYPLTFGGG TKLEIKAAAG SGGSGFWVLV VVGGVLACYS LLVTVAFIIF |
| | | WVRSKRSRLL HSDYMNMTPR RPGPTRKHYQ PYAPPRDFAA YRSKKVAKKP |
| | | TNKAPHPKQE PQEINFPDDL PGSNTAAPVQ ETLHGCQPVT QEDGKESRIS |
| | | VQERQ |
| 91 | CD8a_HuMFE23_ | MALPVTALLL PLALLLHAAR PQVKLEQSGA EVVKPGASVK LSCKASGFNI |
| | CD28TM_ | KDSYMHWLRQ GPGQRLEWIG WIDPENGDTE YAPKFQGKAT FTTDTSANTA |
| | CD28_CD40 | YLGLSSLRPE DTAVYYCNEG TPTGPYYFDY WGQGTLVTVS SGGGGSGGGG |
| | | SGGGGSENVL TQSPSSMSAS VGDRVNIACS ASSSVSYMHW FQQKPGKSPK |
| | | LWIYSTSNLA SGVPSRFSGS GSGTDYSLTI SSMQPEDAAT YYCQQRSSYP |
| | | LTFGGGTKLE IKAAAGSGGS GFWVLVVVGG VLACYSLLVT VAFIIFWVRS |
| | | KRSRLLHSDY MNMTPRRPGP TRKHYQPYAP PRDFAAYRSK KVAKKPTNKA |
| | | PHPKQEPQEI NFPDDLPGSN TAAPVQETLH GCQPVTQEDG KESRISVQER |
| | | Q |
| 92 | CD2_HuMFE23_ | MSFPCKFVAS FLLIFNVSSK GAVSQVKLEQ SGAEVVKPGA SVKLSCKASG |
| | CD28TM_ | FNIKDSYMHW LRQGPGQRLE WIGWIDPENG DTEYAPKFQG KATFTTDTSA |
| | CD28_CD40 | NTAYLGLSSL RPEDTAVYYC NEGTPTGPYY FDYWGQGTLV TVSSGGGGSG |
| | | GGGSGGGGSE NVLTQSPSSM SASVGDRVNI ACSASSSVSY MHWFQQKPGK |
| | | SPKLWIYSTS NLASGVPSRF SGSGSGTDYS LTISSMQPED AATYYCQQRS |
| | | SYPLTFGGGT KLEIKAAAGS GGSGFWVLVV VGGVLACYSL LVTVAFIIFW |
| | | VRSKRSRLLH SDYMNMTPRR PGPTRKHYQP YAPPRDFAAY RSKKVAKKPT |
| | | NKAPHPKQEP QEINFPDDLP GSNTAAPVQE TLHGCQPVTQ EDGKESRISV |
| | | QERQ |
| 93 | IL2_HuMFE23_ | MYRMQLLSCI ALSLALVTNS QVKLEQSGAE VVKPGASVKL SCKASGFNIK |
| | CD28TM_ | DSYMHWLRQG PGQRLEWIGW IDPENGDTEY APKFQGKATF TTDTSANTAY |
| | CD28_CD40 | LGLSSLRPED TAVYYCNEGT PTGPYYFDYW GQGTLVTVSS GGGGSGGGGS |
| | | GGGGSENVLT QSPSSMSASV GDRVNIACSA SSSVSYMHWF QQKPGKSPKL |
| | | WIYSTSNLAS GVPSRFSGSG SGTDYSLTIS SMQPEDAATY YCQQRSSYPL |
| | | TFGGGTKLEI KAAAGSGGSG FWVLVVVGGV LACYSLLVTV AFIIFWVRSK |
| | | RSRLLHSDYM NMTPRRPGPT RKHYQPYAPP RDFAAYRSKK VAKKPTNKAP |
| | | HPKQEPQEIN FPDDLPGSNT AAPVQETLHG CQPVTQEDGK ESRISVQERQ |
| 94 | GM-CSF_ | MWLQSLLLLG TVACSISQVK LEQSGAEVVK PGASVKLSCK ASGFNIKDSY |
| | HuMFE23_ | MHWLRQGPGQ RLEWIGWIDP ENGDTEYAPK FQGKATFTTD TSANTAYLGL |
| | CD28TM_ | SSLRPEDTAV YYCNEGTPTG PYYFDYWGQG TLVTVSSGGG GSGGGGSGGG |
| | CD28_CD40 | GSENVLTQSP SSMSASVGDR VNIACSASSS VSYMHWFQQK PGKSPKLWIY |
| | | STSNLASGVP SRFSGSGSGT DYSLTISSMQ PEDAATYYCQ QRSSYPLTFG |
| | | GGTKLEIKAA AGSGGSGFWV LVVVGGVLAC YSLLVTVAFI IFWVRSKRSR |
| | | LLHSDYMNMT PRRPGPTRKH YQPYAPPRDF AAYRSKKVAK KPTNKAPHPK |
| | | QEPQEINFPD DLPGSNTAAP VQETLHGCQP VTQEDGKESR ISVQERQ |
| 95 | hIgGk-VIII_ | MEAPAQLLFL LLLWLPDTTR QVKLEQSGAE VVKPGASVKL SCKASGFNIK |
| | HuMFE23_ | DSYMHWLRQG PGQRLEWIGW IDPENGDTEY APKFQGKATF TTDTSANTAY |
| | CD28TM_ | LGLSSLRPED TAVYYCNEGT PTGPYYFDYW GQGTLVTVSS GGGGSGGGGS |
| | CD28_CD40 | GGGGSENVLT QSPSSMSASV GDRVNIACSA SSSVSYMHWF QQKPGKSPKL |
| | | WIYSTSNLAS GVPSRFSGSG SGTDYSLTIS SMQPEDAATY YCQQRSSYPL |
| | | TFGGGTKLEI KAAAGSGGSG FWVLVVVGGV LACYSLLVTV AFIIFWVRSK |
| | | RSRLLHSDYM NMTPRRPGPT RKHYQPYAPP RDFAAYRSKK VAKKPTNKAP |
| | | HPKQEPQEIN FPDDLPGSNT AAPVQETLHG CQPVTQEDGK ESRISVQERQ |
| 96 | OSM_CEA6_ | MGVLLTQRTL LSLVLALLFP SMASMQVQLV QSGAEVKKPG SSVKVSCKAS |
| | spCD28_ | GGTFSNSPIN WLRQAPGQGL EWMGSIIPSF GTANYAQKFQ GRLTITADES |
| | CD28_CD40 | TSTAYMELSS LRSEDTAVYY CAGRSHNYEL YYYYMDVWGQ GTMVTVSSGG |
| | CTP221 | GGSGGGGSGG GGSDIQMTQS PSTLSASIGD RVTITCRASE GIYHWLAWYQ |
| | | QKPGKAPKLL IYKASSLASG APSRFSGSGS GTDFTLTISS LQPDDFATYY |
| | | CQQYSNYPLT FGGGTKLEIK RAAAGSGGSG ILVKQSPMLV AYDNAVNLSC |
| | | KYSYNLFSRE FRASLHKGLD SAVEVCVVYG NYSQQLQVYS KTGFNCDGKL |
| | | GNESVTFYLQ NLYVNQTDIY FCKIEVMYPP PYLDNEKSNG TIIHVKGKHL |
| | | CPSPLFPGPS KPFWVLVVVG GVLACYSLLV TVAFIIFWVR SKRSRLLHSD |
| | | YMNMTPRRPG PTRKHYQPYA PPRDFAAYRS KKVAKKPTNK APHPKQEPQE |
| | | INFPDDLPGS NTAAPVQETL HGCQPVTQED GKESRISVQE RQ |
| 97 | CD8a_CEA6_ | MALPVTALLL PLALLLHAAR PQVQLVQSGA EVKKPGSSVK VSCKASGGTF |
| | spCD28_ | SNSPINWLRQ APGQGLEWMG SIIPSFGTAN YAQKFQGRLT ITADESTSTA |
| | CD28_CD40 | YMELSSLRSE DTAVYYCAGR SHNYELYYYY MDVWGQGTMV TVSSGGGGSG |
| | | GGGSGGGGSD IQMTQSPSTL SASIGDRVTI TGRASEGIYH WLAWYQQKPG |
| | | KAPKLLIYKA SSLASGAPSR FSGSGSGTDF TLTISSLQPD DFATYYCQQY |
| | | SNYPLTFGGG TKLEIKRAAA GSGGSGILVK QSPMLVAYDN AVNLSCKYSY |
| | | NLFSREFRAS LHKGLDSAVE VCVVYGNYSQ QLQVYSKTGF NCDGKLGNES |
| | | VTFYLQNLYV NQTDIYFCKI EVMYPPPYLD NEKSNGTIIH VKGKHLCPSP |
| | | LFPGPSKPFW VLVVVGGVLA CYSLLVTVAF IIFWVRSKRS RLLHSDYMNM |
| | | TPRRPGPTRK HYQPYAPPRD FAAYRSKKVA KKPTNKAPHP KQEPQEINFP |
| | | DDLPGSNTAA PVQETLHGCQ PVTQEDGKES RISVQERQ |
| 98 | CD2_CEA6_ | MSFPCKFVAS FLLIFNVSSK GAVSQVQLVQ SGAEVKKPGS SVKVSCKASG |
| | spCD28_ | GTFSNSPINW LRQAPGQGLE WMGSIIPSFG TANYAQKFQG RLTITADEST |
| | CD28_CD40 | STAYMELSSL RSEDTAVYYC AGRSHNYELY YYYMDVWGQG TMVTVSSGGG |
| | | GSGGGGSGGG GSDIQMTQSP STLSASIGDR VTITCRASEG IYHWLAWYQQ |
| | | KPGKAPKLLI YKASSLASGA PSRFSGSGSG TDFTLTISSL QPDDFATYYC |
| | | QQYSNYPLTF GGGTKLEIKR AAAGSGGSGI LVKQSPMLVA YDNAVNLSCK |
| | | YSYNLFSREF RASLHKGLDS AVEVCVVYGN YSQQLQVYSK TGFNCDGKLG |
| | | NESVTFYLQN LYVNQTDIYF CKIEVMYPPP YLDNEKSNGT IIHVKGKHLC |
| | | PSPLFPGPSK PFWVLVVVGG VLACYSLLVT VAFIIFWVRS KRSRLLHSDY |
| | | MNMTPRRPGP TRKHYQPYAP PRDFAAYRSK KVAKKPTNKA PHPKQEPQEI |
| | | NFPDDLPGSN TAAPVQETLH GCQPVTQEDG KESRISVQER Q |
| 99 | IL2_CEA6_ | MYRMQLLSCI ALSLALVTNS QVQLVQSGAE VKKPGSSVKV SCKASGGTFS |
| | spCD28_ | NSPINWLRQA PGQGLEWMGS IIPSFGTANY AQKFQGRLTI TADESTSTAY |
| | CD28_CD40 | MELSSLRSED TAVYYCAGRS HNYELYYYYM DVWGQGTMVT VSSGGGGSGG |
| | | GGSGGGGSDI QMTQSPSTLS ASIGDRVTIT CRASEGIYHW LAWYQQKPGK |
| | | APKLLIYKAS SLASGAPSRF SGSGSGTDFT LTISSLQPDD FATYYCQQYS |
| | | NYPLTFGGGT KLEIKRAAAG SGGSGILVKQ SPMLVAYDNA VNLSCKYSYN |
| | | LFSREFRASL HKGLDSAVEV CVVYGNYSQQ LQVYSKTGFN CDGKLGNESV |
| | | TFYLQNLYVN QTDIYFCKIE VMYPPPYLDN EKSNGTIIHV KGKHLCPSPL |
| | | FPGPSKPFWV LVVVGGVLAC YSLLVTVAFI IFWVRSKRSR LLHSDYMNMT |
| | | PRRPGPTRKH YQPYAPPRDF AAYRSKKVAK KPTNKAPHPK QEPQEINFPD |
| | | DLPGSNTAAP VQETLHGCQP VTQEDGKESR ISVQERQ |
| 100 | GM-CSF_CEA6_ | MWLQSLLLLG TVACSISQVQ LVQSGAEVKK PGSSVKVSCK ASGGTFSNSP |
| | spCD28_ | INWLRQAPGQ GLEWMGSIIP SFGTANYAQK FQGRLTITAD ESTSTAYMEL |
| | CD28_CD40 | SSLRSEDTAV YYCAGRSHNY ELYYYYMDVW GQGTMVTVSS GGGGSGGGGS |
| | | GGGGSDIQMT QSPSTLSASI GDRVTITCRA SEGIYHWLAW YQQKPGKAPK |
| | | LLIYKASSLA SGAPSRFSGS GSGTDFTLTI SSLQPDDFAT YYCQQYSNYP |
| | | LTFGGGTKLE IKRAAAGSGG SGILVKQSPM LVAYDNAVNL SCKYSYNLFS |
| | | REFRASLHKG LDSAVEVCVV YGNYSQQLQV YSKTGFNCDG KLGNESVTFY |
| | | LQNLYVNQTD IYFCKIEVMY PPPYLDNEKS NGTIIHVKGK HLCPSPLFPG |
| | | PSKPFWVLVV VGGVLACYSL LVTVAFIIFW VRSKRSRLLH SDYMNMTPRR |
| | | PGPTRKHYQP YAPPRDFAAY RSKKVAKKPT NKAPHPKQEP QEINFPDDLP |
| | | GSNTAAPVQE TLHGCQPVTQ EDGKESRISV QERQ |
| 101 | hIgGk-VIII_ | MEAPAQLLFL LLLWLPDTTR QVQLVQSGAE VKKPGSSVKV SCKASGGTFS |
| | CEA6_spCD28_ | NSPINWLRQA PGQGLEWMGS IIPSFGTANY AQKFQGRLTI TADESTSTAY |
| | CD28_CD40 | MELSSLRSED TAVYYCAGRS HNYELYYYYM DVWGQGTMVT VSSGGGGSGG |
| | | GGSGGGGSDI QMTQSPSTLS ASIGDRVTIT CRASEGIYHW LAWYQQKPGK |
| | | APKLLIYKAS SLASGAPSRF SGSGSGTDFT LTISSLQPDD FATYYCQQYS |
| | | NYPLTFGGGT KLEIKRAAAG SGGSGILVKQ SPMLVAYDNA VNLSCKYSYN |
| | | LFSREFRASL HKGLDSAVEV CVVYGNYSQQ LQVYSKTGFN CDGKLGNESV |
| | | TFYLQNLYVN QTDIYFCKIE VMYPPPYLDN EKSNGTIIHV KGKHLCPSPL |
| | | FPGPSKPFWV LVVVGGVLAC YSLLVTVAFI IFWVRSKRSR LLHSDYMNMT |
| | | PRRPGPTRKH YQPYAPPRDF AAYRSKKVAK KPTNKAPHPK QEPQEINFPD |
| | | DLPGSNTAAP VQETLHGCQP VTQEDGKESR ISVQERQ |
| 102 | OSM_CEA6_ | MGVLLTQRTL LSLVLALLFP SMASMQVQLV QSGAEVKKPG SSVKVSCKAS |
| | spCD8_ | GGTFSNSPIN WLRQAPGQGL EWMGSIIPSF GTANYAQKFQ GRLTITADES |
| | CD28_CD40 | TSTAYMELSS LRSEDTAVYY CAGRSHNYEL YYYYMDVWGQ GTMVTVSSGG |
| | | GGSGGGGSGG GGSDIQMTQS PSTLSASIGD RVTITCRASE GIYHWLAWYQ |
| | | QKPGKAPKLL IYKASSLASG APSRFSGSGS GTDFTLTISS LQPDDFATYY |
| | | CQQYSNYPLT FGGGTKLEIK RAAAGSGGSG FVPVFLPAKP TTTPAPRPPT |
| | | PAPTIASQPL SLRPEACRPA AGGAVHTRGL DFACDIYIWA PLAGTCGVLL |
| | | LSLVITLYCN HRNRSKRSRL LHSDYMNMTP RRPGPTRKHY QPYAPPRDFA |
| | | AYRSKKVAKK PTNKAPHPKQ EPQEINFPDD LPGSNTAAPV QETLHGCQPV |
| | | TQEDGKESRI SVQERQ |
| 103 | CD8a_CEA6_ | MALPVTALLL PLALLLHAAR PQVQLVQSGA EVKKPGSSVK VSCKASGGTF |
| | spCD8_ | SNSPINWLRQ APGQGLEWMG SIIPSFGTAN YAQKFQGRLT ITADESTSTA |
| | CD28_CD40 | YMELSSLRSE DTAVYYCAGR SHNYELYYYY MDVWGQGTMV TVSSGGGGSG |
| | | GGGSGGGGSD IQMTQSPSTL SASIGDRVTI TGRASEGIYH WLAWYQQKPG |
| | | KAPKLLIYKA SSLASGAPSR FSGSGSGTDF TLTISSLQPD DFATYYCQQY |
| | | SNYPLTFGGG TKLEIKRAAA GSGGSGFVPV FLPAKPTTTP APRPPTPAPT |
| | | IASQPLSLRP EACRPAAGGA VHTRGLDFAC DIYIWAPLAG TCGVLLLSLV |
| | | ITLYCNHRNR SKRSRLLHSD YMNMTPRRPG PTRKHYQPYA PPRDFAAYRS |
| | | KKVAKKPTNK APHPKQEPQE INFPDDLPGS NTAAPVQETL HGCQPVTQED |
| | | GKESRISVQE RQ |
| 104 | CD2_CEA6_ | MSFPCKFVAS FLLIFNVSSK GAVSQVQLVQ SGAEVKKPGS SVKVSCKASG |
| | spCD8_ | GTFSNSPINW LRQAPGQGLE WMGSIIPSFG TANYAQKFQG RLTITADEST |
| | CD28_CD40 | STAYMELSSL RSEDTAVYYC AGRSHNYELY YYYMDVWGQG TMVTVSSGGG |
| | | GSGGGGSGGG GSDIQMTQSP STLSASIGDR VTITCRASEG IYHWLAWYQQ |
| | | KPGKAPKLLI YKASSLASGA PSRFSGSGSG TDFTLTISSL QPDDFATYYC |
| | | QQYSNYPLTF GGGTKLEIKR AAAGSGGSGF VPVFLPAKPT TTPAPRPPTP |
| | | APTIASQPLS LRPEACRPAA GGAVHTRGLD FACDIYIWAP LAGTCGVLLL |
| | | SLVITLYCNH RNRSKRSRLL HSDYMNMTPR RPGPTRKHYQ PYAPPRDFAA |
| | | YRSKKVAKKP TNKAPHPKQE PQEINFPDDL PGSNTAAPVQ ETLHGCQPVT |
| | | QEDGKESRIS VQERQ |
| 105 | IL2_CEA6_ | MYRMQLLSCI ALSLALVTNS QVQLVQSGAE VKKPGSSVKV SCKASGGTFS |
| | spCD8_ | NSPINWLRQA PGQGLEWMGS IIPSFGTANY AQKFQGRLTI TADESTSTAY |
| | CD28_CD40 | MELSSLRSED TAVYYCAGRS HNYELYYYYM DVWGQGTMVT VSSGGGGSGG |
| | | GGSGGGGSDI QMTQSPSTLS ASIGDRVTIT CRASEGIYHW LAWYQQKPGK |
| | | APKLLIYKAS SLASGAPSRF SGSGSGTDFT LTISSLQPDD FATYYCQQYS |
| | | NYPLTFGGGT KLEIKRAAAG SGGSGFVPVF LPAKPTTTPA PRPPTPAPTI |
| | | ASQPLSLRPE ACRPAAGGAV HTRGLDFACD IYIWAPLAGT CGVLLLSLVI |
| | | TLYCNHRNRS KRSRLLHSDY MNMTPRRPGP TRKHYQPYAP PRDFAAYRSK |
| | | KVAKKPTNKA PHPKQEPQEI NFPDDLPGSN TAAPVQETLH GCQPVTQEDG |
| | | KESRISVQER Q |
| 106 | GM-CSFCEA_ | MWLQSLLLLG TVACSISQVQ LVQSGAEVKK PGSSVKVSCK ASGGTFSNSP |
| | spCD8_ | INWLRQAPGQ GLEWMGSIIP SFGTANYAQK FQGRLTITAD ESTSTAYMEL |
| | CD28_CD40 | SSLRSEDTAV YYCAGRSHNY ELYYYYMDVW GQGTMVTVSS GGGGSGGGGS |
| | | GGGGSDIQMT QSPSTLSASI GDRVTITCRA SEGIYHWLAW YQQKPGKAPK |
| | | LLIYKASSLA SGAPSRFSGS GSGTDFTLTI SSLQPDDFAT YYCQQYSNYP |
| | | LTFGGGTKLE IKRAAAGSGG SGFVPVFLPA KPTTTPAPRP PTPAPTIASQ |
| | | PLSLRPEACR PAAGGAVHTR GLDFACDIYI WAPLAGTCGV LLLSLVITLY |
| | | CNHRNRSKRS RLLHSDYMNM TPRRPGPTRK HYQPYAPPRD FAAYRSKKVA |
| | | KKPTNKAPHP KQEPQEINFP DDLPGSNTAA PVQETLHGCQ PVTQEDGKES |
| | | RISVQERQ |
| 107 | hIgGk-VIII_ | MEAPAQLLFL LLLWLPDTTR QVQLVQSGAE VKKPGSSVKV SCKASGGTFS |
| | CEA6_ | NSPINWLRQA PGQGLEWMGS IIPSFGTANY AQKFQGRLTI TADESTSTAY |
| | spCD8_ | MELSSLRSED TAVYYCAGRS HNYELYYYYM DVWGQGTMVT VSSGGGGSGG |
| | CD28_CD40 | GGSGGGGSDI QMTQSPSTLS ASIGDRVTIT CRASEGIYHW LAWYQQKPGK |
| | | APKLLIYKAS SLASGAPSRF SGSGSGTDFT LTISSLQPDD FATYYCQQYS |
| | | NYPLTFGGGT KLEIKRAAAG SGGSGFVPVF LPAKPTTTPA PRPPTPAPTI |
| | | ASQPLSLRPE ACRPAAGGAV HTRGLDFACD IYIWAPLAGT CGVLLLSLVI |
| | | TLYCNHRNRS KRSRLLHSDY MNMTPRRPGP TRKHYQPYAP PRDFAAYRSK |
| | | KVAKKPTNKA PHPKQEPQEI NFPDDLPGSN TAAPVQETLH GCQPVTQEDG |
| | | KESRISVQER Q |
| 108 | OSM_CEA6_ | MGVLLTQRTL LSLVLALLFP SMASMQVQLV QSGAEVKKPG SSVKVSCKAS |
| | CD28TM_ | GGTFSNSPIN WLRQAPGQGL EWMGSIIPSF GTANYAQKFQ GRLTITADES |
| | CD28_CD40 | TSTAYMELSS LRSEDTAVYY CAGRSHNYEL YYYYMDVWGQ GTMVTVSSGG |
| | | GGSGGGGSGG GGSDIQMTQS PSTLSASIGD RVTITCRASE GIYHWLAWYQ |
| | | QKPGKAPKLL IYKASSLASG APSRFSGSGS GTDFTLTISS LQPDDFATYY |
| | | CQQYSNYPLT FGGGTKLEIK RAAAGSGGSG FWVLVVVGGV LACYSLLVTV |
| | | AFIIFWVRSK RSRLLHSDYM NMTPRRPGPT RKHYQPYAPP RDFAAYRSKK |
| | | VAKKPTNKAP HPKQEPQEIN FPDDLPGSNT AAPVQETLHG CQPVTQEDGK |
| | | ESRISVQERQ |
| 109 | CD8a_CEA6_ | MALPVTALLL PLALLLHAAR PQVQLVQSGA EVKKPGSSVK VSCKASGGTF |
| | CD28TM_ | SNSPINWLRQ APGQGLEWMG SIIPSFGTAN YAQKFQGRLT ITADESTSTA |
| | CD28_CD40 | YMELSSLRSE DTAVYYCAGR SHNYELYYYY MDVWGQGTMV TVSSGGGGSG |
| | | GGGSGGGGSD IQMTQSPSTL SASIGDRVTI TCRASEGIYH WLAWYQQKPG |
| | | KAPKLLIYKA SSLASGAPSR FSGSGSGTDF TLTISSLQPD DFATYYCQQY |
| | | SNYPLTFGGG TKLEIKRAAA GSGGSGFWVL VVVGGVLACY SLLVTVAFII |
| | | FWVRSKRSRL LHSDYMNMTP RRPGPTRKHY QPYAPPRDFA AYRSKKVAKK |
| | | PTNKAPHPKQ EPQEINFPDD LPGSNTAAPV QETLHGCQPV TQEDGKESRI |
| | | SVQERQ |
| 110 | CD2_CEA6_ | MSFPCKFVAS FLLIFNVSSK GAVSQVQLVQ SGAEVKKPGS SVKVSCKASG |
| | CD28TM_ | GTFSNSPINW LRQAPGQGLE WMGSIIPSFG TANYAQKFQG RLTITADEST |
| | CD28_CD40 | STAYMELSSL RSEDTAVYYC AGRSHNYELY YYYMDVWGQG TMVTVSSGGG |
| | | GSGGGGSGGG GSDIQMTQSP STLSASIGDR VTITCRASEG IYHWLAWYQQ |
| | | KPGKAPKLLI YKASSLASGA PSRFSGSGSG TDFTLTISSL QPDDFATYYC |
| | | QQYSNYPLTF GGGTKLEIKR AAAGSGGSGF WVLVVVGGVL ACYSLLVTVA |
| | | FIIFWVRSKR SRLLHSDYMN MTPRRPGPTR KHYQPYAPPR DFAAYRSKKV |
| | | AKKPTNKAPH PKQEPQEINF PDDLPGSNTA APVQETLHGC QPVTQEDGKE |
| | | SRISVQERQ |
| 111 | IL2_CEA6_ | MYRMQLLSCI ALSLALVTNS QVQLVQSGAE VKKPGSSVKV SCKASGGTFS |
| | CD28TM_ | NSPINWLRQA PGQGLEWMGS IIPSFGTANY AQKFQGRLTI TADESTSTAY |
| | CD28_CD40 | MELSSLRSED TAVYYCAGRS HNYELYYYYM DVWGQGTMVT VSSGGGGSGG |
| | | GGSGGGGSDI QMTQSPSTLS ASIGDRVTIT CRASEGIYHW LAWYQQKPGK |
| | | APKLLIYKAS SLASGAPSRF SGSGSGTDFT LTISSLQPDD FATYYCQQYS |
| | | NYPLTFGGGT KLEIKRAAAG SGGSGFWVLV VVGGVLACYS LLVTVAFIIF |
| | | WVRSKRSRLL HSDYMNMTPR RPGPTRKHYQ PYAPPRDFAA YRSKKVAKKP |
| | | TNKAPHPKQE PQEINFPDDL PGSNTAAPVQ ETLHGCQPVT QEDGKESRIS |
| | | VQERQ |
| 112 | GM-CSF_CEA6_ | MWLQSLLLLG TVACSISQVQ LVQSGAEVKK PGSSVKVSCK ASGGTFSNSP |
| | CD28TM_ | INWLRQAPGQ GLEWMGSIIP SFGTANYAQK FQGRLTITAD ESTSTAYMEL |
| | CD28_CD40 | SSLRSEDTAV YYCAGRSHNY ELYYYYMDVW GQGTMVTVSS GGGGSGGGGS |
| | | GGGGSDIQMT QSPSTLSASI GDRVTITCRA SEGIYHWLAW YQQKPGKAPK |
| | | LLIYKASSLA SGAPSRFSGS GSGTDFTLTI SSLQPDDFAT YYCQQYSNYP |
| | | LTFGGGTKLE IKRAAAGSGG SGFWVLVVVG GVLACYSLLV TVAFIIFWVR |
| | | SKRSRLLHSD YMNMTPRRPG PTRKHYQPYA PPRDFAAYRS KKVAKKPTNK |
| | | APHPKQEPQE INFPDDLPGS NTAAPVQETL HGCQPVTQED GKESRISVQE |
| | | RQ |
| 113 | hIgGk-VIII_ | MEAPAQLLFL LLLWLPDTTR QVQLVQSGAE VKKPGSSVKV SCKASGGTFS |
| | CEA6_ | NSPINWLRQA PGQGLEWMGS IIPSFGTANY AQKFQGRLTI TADESTSTAY |
| | CD28TM_ | MELSSLRSED TAVYYCAGRS HNYELYYYYM DVWGQGTMVT VSSGGGGSGG |
| | CD28_CD40 | GGSGGGGSDI QMTQSPSTLS ASIGDRVTIT CRASEGIYHW LAWYQQKPGK |
| | | APKLLIYKAS SLASGAPSRF SGSGSGTDFT LTISSLQPDD FATYYCQQYS |
| | | NYPLTFGGGT KLEIKRAAAG SGGSGFWVLV VVGGVLACYS LLVTVAFIIF |
| | | WVRSKRSRLL HSDYMNMTPR RPGPTRKHYQ PYAPPRDFAA YRSKKVAKKP |
| | | TNKAPHPKQE PQEINFPDDL PGSNTAAPVQ ETLHGCQPVT QEDGKESRIS |
| | | VQERQ |
| 114 | OSM_BW431/26_ | MGVLLTQRTL LSLVLALLFP SMASMQLQES GPGLVRPSQT LSLTCTVSGF |
| | spCD28_ | TISSGYSWHW VRQPPGRGLE WIGYIQYSGI TNYNPSLKSR VTMLVDTSKN |
| | CD28_CD40 | QFSLRLSSVT AADTAVYYCA REDYDYHWYF DVWGQGSLVT VSSGGGGSGG |
| | CTP222 | GGSGGGGSGV HSDIQMTQSP SSLSASVGDR VTITCSTSSS VSYMHWYQQK |
| | | PGKAPKLLIY STSNLASGVP SRFSGSGSGT DFTFTISSLQ PEDIATYYCH |
| | | QWSSYPTFGQ GTKVEIKRAA AGSGGSGILV KQSPMLVAYD NAVNLSCKYS |
| | | YNLFSREFRA SLHKGLDSAV EVCVVYGNYS QQLQVYSKTG FNCDGKLGNE |
| | | SVTFYLQNLY VNQTDIYFCK IEVMYPPPYL DNEKSNGTII HVKGKHLCPS |
| | | PLFPGPSKPF WVLVVVGGVL ACYSLLVTVA FIIFWVRSKR SRLLHSDYMN |
| | | MTPRRPGPTR KHYQPYAPPR DFAAYRSKKV AKKPTNKAPH PKQEPQEINF |
| | | PDDLPGSNTA APVQETLHGC QPVTQEDGKE SRISVQERQ |
| 115 | CD8a_BW431/26_ | MALPVTALLL PLALLLHAAR PQLQESGPGL VRPSQTLSLT CTVSGFTISS |
| | spCD28_ | GYSWHWVRQP PGRGLEWIGY IQYSGITNYN PSLKSRVTML VDTSKNQFSL |
| | CD28_CD40 | RLSSVTAADT AVYYCAREDY DYHWYFDVWG QGSLVTVSSG GGGSGGGGSG |
| | | GGGSGVHSDI QMTQSPSSLS ASVGDRVTIT CSTSSSVSYM HWYQQKPGKA |
| | | PKLLIYSTSN LASGVPSRFS GSGSGTDFTF TISSLQPEDI ATYYCHQWSS |
| | | YPTFGQGTKV EIKRAAAGSG GSGILVKQSP MLVAYDNAVN LSCKYSYNLF |
| | | SREFRASLHK GLDSAVEVCV VYGNYSQQLQ VYSKTGFNCD GKLGNESVTF |
| | | YLQNLYVNQT DIYFCKIEVM YPPPYLDNEK SNGTIIHVKG KHLCPSPLFP |
| | | GPSKPFWVLV VVGGVLACYS LLVTVAFIIF WVRSKRSRLL HSDYMNMTPR |
| | | RPGPTRKHYQ PYAPPRDFAA YRSKKVAKKP TNKAPHPKQE PQEINFPDDL |
| | | PGSNTAAPVQ ETLHGCQPVT QEDGKESRIS VQERQ |
| 116 | CD2_BW431/26_ | MSFPCKFVAS FLLIFNVSSK GAVSQLQESG PGLVRPSQTL SLTCTVSGFT |
| | spCD28_ | ISSGYSWHWV RQPPGRGLEW IGYIQYSGIT NYNPSLKSRV TMLVDTSKNQ |
| | CD28_CD40 | FSLRLSSVTA ADTAVYYCAR EDYDYHWYFD VWGQGSLVTV SSGGGGSGGG |
| | | GSGGGGSGVH SDIQMTQSPS SLSASVGDRV TITCSTSSSV SYMHWYQQKP |
| | | GKAPKLLIYS TSNLASGVPS RFSGSGSGTD FTFTISSLQP EDIATYYCHQ |
| | | WSSYPTFGQG TKVEIKRAAA GSGGSGILVK QSPMLVAYDN AVNLSCKYSY |
| | | NLFSREFRAS LHKGLDSAVE VCVVYGNYSQ QLQVYSKTGF NCDGKLGNES |
| | | VTFYLQNLYV NQTDIYFCKI EVMYPPPYLD NEKSNGTIIH VKGKHLCPSP |
| | | LFPGPSKPFW VLVVVGGVLA CYSLLVTVAF IIFWVRSKRS RLLHSDYMNM |
| | | TPRRPGPTRK HYQPYAPPRD FAAYRSKKVA KKPTNKAPHP KQEPQEINFP |
| | | DDLPGSNTAA PVQETLHGCQ PVTQEDGKES RISVQERQ |
| 117 | IL2_BW431/26_ | MYRMQLLSCI ALSLALVTNS QLQESGPGLV RPSQTLSLTC TVSGFTISSG |
| | spCD28_ | YSWHWVRQPP GRGLEWIGYI QYSGITNYNP SLKSRVTMLV DTSKNQFSLR |
| | CD28_CD40 | LSSVTAADTA VYYCAREDYD YHWYFDVWGQ GSLVTVSSGG GGSGGGGSGG |
| | | GGSGVHSDIQ MTQSPSSLSA SVGDRVTITC STSSSVSYMH WYQQKPGKAP |
| | | KLLIYSTSNL ASGVPSRFSG SGSGTDFTFT ISSLQPEDIA TYYCHQWSSY |
| | | PTFGQGTKVE IKRAAAGSGG SGILVKQSPM LVAYDNAVNL SCKYSYNLFS |
| | | REFRASLHKG LDSAVEVCVV YGNYSQQLQV YSKTGFNCDG KLGNESVTFY |
| | | LQNLYVNQTD IYFCKIEVMY PPPYLDNEKS NGTIIHVKGK HLCPSPLFPG |
| | | PSKPFWVLVV VGGVLACYSL LVTVAFIIFW VRSKRSRLLH SDYMNMTPRR |
| | | PGPTRKHYQP YAPPRDFAAY RSKKVAKKPT NKAPHPKQEP QEINFPDDLP |
| | | GSNTAAPVQE TLHGCQPVTQ EDGKESRISV QERQ |
| 118 | GM-CSF_ | MWLQSLLLLG TVACSISQLQ ESGPGLVRPS QTLSLTCTVS GFTISSGYSW |
| | BW431/26_ | HWVRQPPGRG LEWIGYIQYS GITNYNPSLK SRVTMLVDTS KNQFSLRLSS |
| | spCD28_ | VTAADTAVYY CAREDYDYHW YFDVWGQGSL VTVSSGGGGS GGGGSGGGGS |
| | CD28_CD40 | GVHSDIQMTQ SPSSLSASVG DRVTITCSTS SSVSYMHWYQ QKPGKAPKLL |
| | | IYSTSNLASG VPSRFSGSGS GTDFTFTISS LQPEDIATYY CHQWSSYPTF |
| | | GQGTKVEIKR AAAGSGGSGI LVKQSPMLVA YDNAVNLSCK YSYNLFSREF |
| | | RASLHKGLDS AVEVCVVYGN YSQQLQVYSK TGFNCDGKLG NESVTFYLQN |
| | | LYVNQTDIYF CKIEVMYPPP YLDNEKSNGT IIHVKGKHLC PSPLFPGPSK |
| | | PFWVLVVVGG VLACYSLLVT VAFIIFWVRS KRSRLLHSDY MNMTPRRPGP |
| | | TRKHYQPYAP PRDFAAYRSK KVAKKPTNKA PHPKQEPQEI NFPDDLPGSN |
| | | TAAPVQETLH GCQPVTQEDG KESRISVQER Q |
| 119 | hIgGk-VIII_ | MEAPAQLLFL LLLWLPDTTR QLQESGPGLV RPSQTLSLTC TVSGFTISSG |
| | BW431/26_ | YSWHWVRQPP GRGLEWIGYI QYSGITNYNP SLKSRVTMLV DTSKNQFSLR |
| | spCD28_ | LSSVTAADTA VYYCAREDYD YHWYFDVWGQ GSLVTVSSGG GGSGGGGSGG |
| | CD28_CD40 | GGSGVHSDIQ MTQSPSSLSA SVGDRVTITC STSSSVSYMH WYQQKPGKAP |
| | | KLLIYSTSNL ASGVPSRFSG SGSGTDFTFT ISSLQPEDIA TYYCHQWSSY |
| | | PTFGQGTKVE IKRAAAGSGG SGILVKQSPM LVAYDNAVNL SCKYSYNLFS |
| | | REFRASLHKG LDSAVEVCVV YGNYSQQLQV YSKTGFNCDG KLGNESVTFY |
| | | LQNLYVNQTD IYFCKIEVMY PPPYLDNEKS NGTIIHVKGK HLCPSPLFPG |
| | | PSKPFWVLVV VGGVLACYSL LVTVAFIIFW VRSKRSRLLH SDYMNMTPRR |
| | | PGPTRKHYQP YAPPRDFAAY RSKKVAKKPT NKAPHPKQEP QEINFPDDLP |
| | | GSNTAAPVQE TLHGCQPVTQ EDGKESRISV QERQ |
| 120 | OSM_BW431/26_ | MGVLLTQRTL LSLVLALLFP SMASMQLQES GPGLVRPSQT LSLTCTVSGF |
| | spCD8_ | TISSGYSWHW VRQPPGRGLE WIGYIQYSGI TNYNPSLKSR VTMLVDTSKN |
| | CD28_CD40 | QFSLRLSSVT AADTAVYYCA REDYDYHWYF DVWGQGSLVT VSSGGGGSGG |
| | | GGSGGGGSGV HSDIQMTQSP SSLSASVGDR VTITCSTSSS VSYMHWYQQK |
| | | PGKAPKLLIY STSNLASGVP SRFSGSGSGT DFTFTISSLQ PEDIATYYCH |
| | | QWSSYPTFGQ GTKVEIKRAA AGSGGSGFVP VFLPAKPTTT PAPRPPTPAP |
| | | TIASQPLSLR PEACRPAAGG AVHTRGLDFA CDIYIWAPLA GTCGVLLLSL |
| | | VITLYCNHRN RSKRSRLLHS DYMNMTPRRP GPTRKHYQPY APPRDFAAYR |
| | | SKKVAKKPTN KAPHPKQEPQ EINFPDDLPG SNTAAPVQET LHGCQPVTQE |
| | | DGKESRISVQ ERQ |
| 121 | CD8a_BW431/26_ | MALPVTALLL PLALLLHAAR PQLQESGPGL VRPSQTLSLT CTVSGFTISS |
| | spCD8_ | GYSWHWVRQP PGRGLEWIGY IQYSGITNYN PSLKSRVTML VDTSKNQFSL |
| | CD28_CD40 | RLSSVTAADT AVYYCAREDY DYHWYFDVWG QGSLVTVSSG GGGSGGGGSG |
| | | GGGSGVHSDI QMTQSPSSLS ASVGDRVTIT CSTSSSVSYM HWYQQKPGKA |
| | | PKLLIYSTSN LASGVPSRFS GSGSGTDFTF TISSLQPEDI ATYYCHQWSS |
| | | YPTFGQGTKV EIKRAAAGSG GSGFVPVFLP AKPTTTPAPR PPTPAPTIAS |
| | | QPLSLRPEAC RPAAGGAVHT RGLDFACDIY IWAPLAGTCG VLLLSLVITL |
| | | YCNHRNRSKR SRLLHSDYMN MTPRRPGPTR KHYQPYAPPR DFAAYRSKKV |
| | | AKKPTNKAPH PKQEPQEINF PDDLPGSNTA APVQETLHGC QPVTQEDGKE |
| | | SRISVQERQ |
| 122 | CD2_BW431/26_ | MSFPCKFVAS FLLIFNVSSK GAVSQLQESG PGLVRPSQTL SLTCTVSGFT |
| | spCD8_ | ISSGYSWHWV RQPPGRGLEW IGYIQYSGIT NYNPSLKSRV TMLVDTSKNQ |
| | CD28_CD40 | FSLRLSSVTA ADTAVYYCAR EDYDYHWYFD VWGQGSLVTV SSGGGGSGGG |
| | | GSGGGGSGVH SDIQMTQSPS SLSASVGDRV TITCSTSSSV SYMHWYQQKP |
| | | GKAPKLLIYS TSNLASGVPS RFSGSGSGTD FTFTISSLQP EDIATYYCHQ |
| | | WSSYPTFGQG TKVEIKRAAA GSGGSGFVPV FLPAKPTTTP APRPPTPAPT |
| | | IASQPLSLRP EACRPAAGGA VHTRGLDFAC DIYIWAPLAG TCGVLLLSLV |
| | | ITLYCNHRNR SKRSRLLHSD YMNMTPRRPG PTRKHYQPYA PPRDFAAYRS |
| | | KKVAKKPTNK APHPKQEPQE INFPDDLPGS NTAAPVQETL HGCQPVTQED |
| | | GKESRISVQE RQ |
| 123 | IL2_BW431/26_ | MYRMQLLSCI ALSLALVTNS QLQESGPGLV RPSQTLSLTC TVSGFTISSG |
| | spCD8_ | YSWHWVRQPP GRGLEWIGYI QYSGITNYNP SLKSRVTMLV DTSKNQFSLR |
| | CD28_CD40 | LSSVTAADTA VYYCAREDYD YHWYFDVWGQ GSLVTVSSGG GGSGGGGSGG |
| | | GGSGVHSDIQ MTQSPSSLSA SVGDRVTITC STSSSVSYMH WYQQKPGKAP |
| | | KLLIYSTSNL ASGVPSRFSG SGSGTDFTFT ISSLQPEDIA TYYCHQWSSY |
| | | PTFGQGTKVE IKRAAAGSGG SGFVPVFLPA KPTTTPAPRP PTPAPTIASQ |
| | | PLSLRPEACR PAAGGAVHTR GLDFACDIYI WAPLAGTCGV LLLSLVITLY |
| | | CNHRNRSKRS RLLHSDYMNM TPRRPGPTRK HYQPYAPPRD FAAYRSKKVA |
| | | KKPTNKAPHP KQEPQEINFP DDLPGSNTAA PVQETLHGCQ PVTQEDGKES |
| | | RISVQERQ |
| 124 | GM-CSF_ | MWLQSLLLLG TVACSISQLQ ESGPGLVRPS QTLSLTCTVS GFTISSGYSW |
| | BW431/26_spCD8_ | HWVRQPPGRG LEWIGYIQYS GITNYNPSLK SRVTMLVDTS KNQFSLRLSS |
| | CD28_CD40 | VTAADTAVYY CAREDYDYHW YFDVWGQGSL VTVSSGGGGS GGGGSGGGGS |
| | | GVHSDIQMTQ SPSSLSASVG DRVTITCSTS SSVSYMHWYQ QKPGKAPKLL |
| | | IYSTSNLASG VPSRFSGSGS GTDFTFTISS LQPEDIATYY CHQWSSYPTF |
| | | GQGTKVEIKR AAAGSGGSGF VPVFLPAKPT TTPAPRPPTP APTIASQPLS |
| | | LRPEACRPAA GGAVHTRGLD FACDIYIWAP LAGTCGVLLL SLVITLYCNH |
| | | RNRSKRSRLL HSDYMNMTPR RPGPTRKHYQ PYAPPRDFAA YRSKKVAKKP |
| | | TNKAPHPKQE PQEINFPDDL PGSNTAAPVQ ETLHGCQPVT QEDGKESRIS |
| | | VQERQ |
| 125 | hIgGk- | MEAPAQLLFL LLLWLPDTTR QLQESGPGLV RPSQTLSLTC TVSGFTISSG |
| | VIII_BW431/26_ | YSWHWVRQPP GRGLEWIGYI QYSGITNYNP SLKSRVTMLV DTSKNQFSLR |
| | spCD8_ | LSSVTAADTA VYYCAREDYD YHWYFDVWGQ GSLVTVSSGG GGSGGGGSGG |
| | CD28_CD40 | GGSGVHSDIQ MTQSPSSLSA SVGDRVTITC STSSSVSYMH WYQQKPGKAP |
| | | KLLIYSTSNL ASGVPSRFSG SGSGTDFTFT ISSLQPEDIA TYYCHQWSSY |
| | | PTFGQGTKVE IKRAAAGSGG SGFVPVFLPA KPTTTPAPRP PTPAPTIASQ |
| | | PLSLRPEACR PAAGGAVHTR GLDFACDIYI WAPLAGTCGV LLLSLVITLY |
| | | CNHRNRSKRS RLLHSDYMNM TPRRPGPTRK HYQPYAPPRD FAAYRSKKVA |
| | | KKPTNKAPHP KQEPQEINFP DDLPGSNTAA PVQETLHGCQ PVTQEDGKES |
| | | RISVQERQ |
| 126 | OSM_BW431/26_ | MGVLLTQRTL LSLVLALLFP SMASMQLQES GPGLVRPSQT LSLTCTVSGF |
| | CD28TM_ | TISSGYSWHW VRQPPGRGLE WIGYIQYSGI TNYNPSLKSR VTMLVDTSKN |
| | CD28_CD40 | QFSLRLSSVT AADTAVYYCA REDYDYHWYF DVWGQGSLVT VSSGGGGSGG |
| | | GGSGGGGSGV HSDIQMTQSP SSLSASVGDR VTITCSTSSS VSYMHWYQQK |
| | | PGKAPKLLIY STSNLASGVP SRFSGSGSGT DFTFTISSLQ PEDIATYYCH |
| | | QWSSYPTFGQ GTKVEIKRAA AGSGGSGFWV LVVVGGVLAC YSLLVTVAFI |
| | | IFWVRSKRSR LLHSDYMNMT PRRPGPTRKH YQPYAPPRDF AAYRSKKVAK |
| | | KPTNKAPHPK QEPQEINFPD DLPGSNTAAP VQETLHGCQP VTQEDGKESR |
| | | ISVQERQ |
| 127 | CD8a_BW431/26_ | MALPVTALLL PLALLLHAAR PQLQESGPGL VRPSQTLSLT CTVSGFTISS |
| | CD28TM_ | GYSWHWVRQP PGRGLEWIGY IQYSGITNYN PSLKSRVTML VDTSKNQFSL |
| | CD28_CD40 | RLSSVTAADT AVYYCAREDY DYHWYFDVWG QGSLVTVSSG GGGSGGGGSG |
| | | GGGSGVHSDI QMTQSPSSLS ASVGDRVTIT CSTSSSVSYM HWYQQKPGKA |
| | | PKLLIYSTSN LASGVPSRFS GSGSGTDFTF TISSLQPEDI ATYYCHQWSS |
| | | YPTFGQGTKV EIKRAAAGSG GSGFWVLVVV GGVLACYSLL VTVAFIIFWV |
| | | RSKRSRLLHS DYMNMTPRRP GPTRKHYQPY APPRDFAAYR SKKVAKKPTN |
| | | KAPHPKQEPQ EINFPDDLPG SNTAAPVQET LHGCQPVTQE DGKESRISVQ |
| | | ERQ |
| 128 | CD2_BW431/26_ | MSFPCKFVAS FLLIFNVSSK GAVSQLQESG PGLVRPSQTL SLTCTVSGFT |
| | CD28TM_ | ISSGYSWHWV RQPPGRGLEW IGYIQYSGIT NYNPSLKSRV TMLVDTSKNQ |
| | CD28_CD40 | FSLRLSSVTA ADTAVYYCAR EDYDYHWYFD VWGQGSLVTV SSGGGGSGGG |
| | | GSGGGGSGVH SDIQMTQSPS SLSASVGDRV TITCSTSSSV SYMHWYQQKP |
| | | GKAPKLLIYS TSNLASGVPS RFSGSGSGTD FTFTISSLQP EDIATYYCHQ |
| | | WSSYPTFGQG TKVEIKRAAA GSGGSGFWVL VVVGGVLACY SLLVTVAFII |
| | | FWVRSKRSRL LHSDYMNMTP RRPGPTRKHY QPYAPPRDFA AYRSKKVAKK |
| | | PTNKAPHPKQ EPQEINFPDD LPGSNTAAPV QETLHGCQPV TQEDGKESRI |
| | | SVQERQ |
| 129 | IL2_BW431/26_ | MYRMQLLSCI ALSLALVTNS QLQESGPGLV RPSQTLSLTC TVSGFTISSG |
| | CD28TM_ | YSWHWVRQPP GRGLEWIGYI QYSGITNYNP SLKSRVTMLV DTSKNQFSLR |
| | CD28_CD40 | LSSVTAADTA VYYCAREDYD YHWYFDVWGQ GSLVTVSSGG GGSGGGGSGG |
| | | GGSGVHSDIQ MTQSPSSLSA SVGDRVTITC STSSSVSYMH WYQQKPGKAP |
| | | KLLIYSTSNL ASGVPSRFSG SGSGTDFTFT ISSLQPEDIA TYYCHQWSSY |
| | | PTFGQGTKVE IKRAAAGSGG SGFWVLVVVG GVLACYSLLV TVAFIIFWVR |
| | | SKRSRLLHSD YMNMTPRRPG PTRKHYQPYA PPRDFAAYRS KKVAKKPTNK |
| | | APHPKQEPQE INFPDDLPGS NTAAPVQETL HGCQPVTQED GKESRISVQE |
| | | RQ |
| 130 | GM-CSF_ | MWLQSLLLLG TVACSISQLQ ESGPGLVRPS QTLSLTCTVS GFTISSGYSW |
| | BW431/26_ | HWVRQPPGRG LEWIGYIQYS GITNYNPSLK SRVTMLVDTS KNQFSLRLSS |
| | CD28TM_ | VTAADTAVYY CAREDYDYHW YFDVWGQGSL VTVSSGGGGS GGGGSGGGGS |
| | CD28_CD40 | GVHSDIQMTQ SPSSLSASVG DRVTITCSTS SSVSYMHWYQ QKPGKAPKLL |
| | | IYSTSNLASG VPSRFSGSGS GTDFTFTISS LQPEDIATYY CHQWSSYPTF |
| | | GQGTKVEIKR AAAGSGGSGF WVLVVVGGVL ACYSLLVTVA FIIFWVRSKR |
| | | SRLLHSDYMN MTPRRPGPTR KHYQPYAPPR DFAAYRSKKV AKKPTNKAPH |
| | | PKQEPQEINF PDDLPGSNTA APVQETLHGC QPVTQEDGKE SRISVQERQ |
| 131 | hIgGk-VIII_ | MEAPAQLLFL LLLWLPDTTR QLQESGPGLV RPSQTLSLTC TVSGFTISSG |
| | BW431/26_ | YSWHWVRQPP GRGLEWIGYI QYSGITNYNP SLKSRVTMLV DTSKNQFSLR |
| | CD28TM_ | LSSVTAADTA VYYCAREDYD YHWYFDVWGQ GSLVTVSSGG GGSGGGGSGG |
| | CD28_CD40 | GGSGVHSDIQ MTQSPSSLSA SVGDRVTITC STSSSVSYMH WYQQKPGKAP |
| | | KLLIYSTSNL ASGVPSRFSG SGSGTDFTFT ISSLQPEDIA TYYCHQWSSY |
| | | PTFGQGTKVE IKRAAAGSGG SGFWVLVVVG GVLACYSLLV TVAFIIFWVR |
| | | SKRSRLLHSD YMNMTPRRPG PTRKHYQPYA PPRDFAAYRS KKVAKKPTNK |
| | | APHPKQEPQE INFPDDLPGS NTAAPVQETL HGCQPVTQED GKESRISVQE |
| | | RQ |
| 132 | OSM_HuT84.66_ | MGVLLTQRTL LSLVLALLFP SMASMEVQLV ESGGGLVQPG GSLRLSCAAS |
| | spCD28_ | GFNIKDTYMH WVRQAPGKGL EWVARIDPAN GNSKYADSVK GRFTISADTS |
| | CD28_CD40 | KNTAYLQMNS LRAEDTAVYY CAPFGYYVSD YAMAYWGQGT LVTVSSGGGG |
| | CTP223 | SGGGGSGGGG SDIQLTQSPS SLSASVGDRV TITCRAGESV DIFGVGFLHW |
| | | YQQKPGKAPK LLIYRASNLE SGVPSRFSGS GSRTDFTLTI SSLQPEDFAT |
| | | YYCQQTNEDP YTFGQGTKVE IKAAAGSGGS GILVKQSPML VAYDNAVNLS |
| | | CKYSYNLFSR EFRASLHKGL DSAVEVCVVY GNYSQQLQVY SKTGFNCDGK |
| | | LGNESVTFYL QNLYVNQTDI YFCKIEVMYP PPYLDNEKSN GTIIHVKGKH |
| | | LCPSPLFPGP SKPFWVLVVV GGVLACYSLL VTVAFIIFWV RSKRSRLLHS |
| | | DYMNMTPRRP GPTRKHYQPY APPRDFAAYR SKKVAKKPTN KAPHPKQEPQ |
| | | EINFPDDLPG SNTAAPVQET LHGCQPVTQE DGKESRISVQ ERQ |
| 133 | CD8a_HuT84.66_ | MALPVTALLL PLALLLHAAR PEVQLVESGG GLVQPGGSLR LSCAASGFNI |
| | spCD28_ | KDTYMHWVRQ APGKGLEWVA RIDPANGNSK YADSVKGRFT ISADTSKNTA |
| | CD28_CD40 | YLQMNSLRAE DTAVYYCAPF GYYVSDYAMA YWGQGTLVTV SSGGGGSGGG |
| | | GSGGGGSDIQ LTQSPSSLSA SVGDRVTITC RAGESVDIFG VGFLHWYQQK |
| | | PGKAPKLLIY RASNLESGVP SRFSGSGSRT DFTLTISSLQ PEDFATYYCQ |
| | | QTNEDPYTFG QGTKVEIKAA AGSGGSGILV KQSPMLVAYD NAVNLSCKYS |
| | | YNLFSREFRA SLHKGLDSAV EVCVVYGNYS QQLQVYSKTG FNCDGKLGNE |
| | | SVTFYLQNLY VNQTDIYFCK IEVMYPPPYL DNEKSNGTII HVKGKHLCPS |
| | | PLFPGPSKPF WVLVVVGGVL ACYSLLVTVA FIIFWVRSKR SRLLHSDYMN |
| | | MTPRRPGPTR KHYQPYAPPR DFAAYRSKKV AKKPTNKAPH PKQEPQEINF |
| | | PDDLPGSNTA APVQETLHGC QPVTQEDGKE SRISVQERQ |
| 134 | CD2_HuT84.66_ | MSFPCKFVAS FLLIFNVSSK GAVSEVQLVE SGGGLVQPGG SLRLSCAASG |
| | spCD28_ | FNIKDTYMHW VRQAPGKGLE WVARIDPANG NSKYADSVKG RFTISADTSK |
| | CD28_CD40 | NTAYLQMNSL RAEDTAVYYC APFGYYVSDY AMAYWGQGTL VTVSSGGGGS |
| | | GGGGSGGGGS DIQLTQSPSS LSASVGDRVT ITCRAGESVD IFGVGFLHWY |
| | | QQKPGKAPKL LIYRASNLES GVPSRFSGSG SRTDFTLTIS SLQPEDFATY |
| | | YCQQTNEDPY TFGQGTKVEI KAAAGSGGSG ILVKQSPMLV AYDNAVNLSC |
| | | KYSYNLFSRE FRASLHKGLD SAVEVCVVYG NYSQQLQVYS KTGFNCDGKL |
| | | GNESVTFYLQ NLYVNQTDIY FCKIEVMYPP PYLDNEKSNG TIIHVKGKHL |
| | | CPSPLFPGPS KPFWVLVVVG GVLACYSLLV TVAFIIFWVR SKRSRLLHSD |
| | | YMNMTPRRPG PTRKHYQPYA PPRDFAAYRS KKVAKKPTNK APHPKQEPQE |
| | | INFPDDLPGS NTAAPVQETL HGCQPVTQED GKESRISVQE RQ |
| 135 | IL2_HuT84.66 | MYRMQLLSCI ALSLALVTNS EVQLVESGGG LVQPGGSLRL SCAASGFNIK |
| | _spCD28 | DTYMHWVRQA PGKGLEWVAR IDPANGNSKY ADSVKGRFTI SADTSKNTAY |
| | _CD28_CD40 | LQMNSLRAED TAVYYCAPFG YYVSDYAMAY WGQGTLVTVS SGGGGSGGGG |
| | | SGGGGSDIQL TQSPSSLSAS VGDRVTITCR AGESVDIFGV GFLHWYQQKP |
| | | GKAPKLLIYR ASNLESGVPS RFSGSGSRTD FTLTISSLQP EDFATYYCQQ |
| | | TNEDPYTFGQ GTKVEIKAAA GSGGSGILVK QSPMLVAYDN AVNLSCKYSY |
| | | NLFSREFRAS LHKGLDSAVE VCVVYGNYSQ QLQVYSKTGF NCDGKLGNES |
| | | VTFYLQNLYV NQTDIYFCKI EVMYPPPYLD NEKSNGTIIH VKGKHLCPSP |
| | | LFPGPSKPFW VLVVVGGVLA CYSLLVTVAF IIFWVRSKRS RLLHSDYMNM |
| | | TPRRPGPTRK HYQPYAPPRD FAAYRSKKVA KKPTNKAPHP KQEPQEINFP |
| | | DDLPGSNTAA PVQETLHGCQ PVTQEDGKES RISVQERQ |
| 136 | GM-CSF_ | MWLQSLLLLG TVACSISEVQ LVESGGGLVQ PGGSLRLSCA ASGFNIKDTY |
| | HuT84.66_ | MHWVRQAPGK GLEWVARIDP ANGNSKYADS VKGRFTISAD TSKNTAYLQM |
| | spCD28_ | NSLRAEDTAV YYCAPFGYYV SDYAMAYWGQ GTLVTVSSGG GGSGGGGSGG |
| | CD28_CD40 | GGSDIQLTQS PSSLSASVGD RVTITCRAGE SVDIFGVGFL HWYQQKPGKA |
| | | PKLLIYRASN LESGVPSRFS GSGSRTDFTL TISSLQPEDF ATYYCQQTNE |
| | | DPYTFGQGTK VEIKAAAGSG GSGILVKQSP MLVAYDNAVN LSCKYSYNLF |
| | | SREFRASLHK GLDSAVEVCV VYGNYSQQLQ VYSKTGFNCD GKLGNESVTF |
| | | YLQNLYVNQT DIYFCKIEVM YPPPYLDNEK SNGTIIHVKG KHLCPSPLFP |
| | | GPSKPFWVLV VVGGVLACYS LLVTVAFIIF WVRSKRSRLL HSDYMNMTPR |
| | | RPGPTRKHYQ PYAPPRDFAA YRSKKVAKKP TNKAPHPKQE PQEINFPDDL |
| | | PGSNTAAPVQ ETLHGCQPVT QEDGKESRIS VQERQ |
| 137 | hIgGk- | MEAPAQLLFL LLLWLPDTTR EVQLVESGGG LVQPGGSLRL SCAASGFNIK |
| | VIII_HuT84.66_ | DTYMHWVRQA PGKGLEWVAR IDPANGNSKY ADSVKGRFTI SADTSKNTAY |
| | spCD28_ | LQMNSLRAED TAVYYCAPFG YYVSDYAMAY WGQGTLVTVS SGGGGSGGGG |
| | CD28_CD40 | SGGGGSDIQL TQSPSSLSAS VGDRVTITCR AGESVDIFGV GFLHWYQQKP |
| | | GKAPKLLIYR ASNLESGVPS RFSGSGSRTD FTLTISSLQP EDFATYYCQQ |
| | | TNEDPYTFGQ GTKVEIKAAA GSGGSGILVK QSPMLVAYDN AVNLSCKYSY |
| | | NLFSREFRAS LHKGLDSAVE VCVVYGNYSQ QLQVYSKTGF NCDGKLGNES |
| | | VTFYLQNLYV NQTDIYFCKI EVMYPPPYLD NEKSNGTIIH VKGKHLCPSP |
| | | LFPGPSKPFW VLVVVGGVLA CYSLLVTVAF IIFWVRSKRS RLLHSDYMNM |
| | | TPRRPGPTRK HYQPYAPPRD FAAYRSKKVA KKPTNKAPHP KQEPQEINFP |
| | | DDLPGSNTAA PVQETLHGCQ PVTQEDGKES RISVQERQ |
| 138 | OSM_HuT84.66_ | MGVLLTQRTL LSLVLALLFP SMASMEVQLV ESGGGLVQPG GSLRLSCAAS |
| | spCD8_ | GFNIKDTYMH WVRQAPGKGL EWVARIDPAN GNSKYADSVK GRFTISADTS |
| | CD28_CD40 | KNTAYLQMNS LRAEDTAVYY CAPFGYYVSD YAMAYWGQGT LVTVSSGGGG |
| | | SGGGGSGGGG SDIQLTQSPS SLSASVGDRV TITCRAGESV DIFGVGFLHW |
| | | YQQKPGKAPK LLIYRASNLE SGVPSRFSGS GSRTDFTLTI SSLQPEDFAT |
| | | YYCQQTNEDP YTFGQGTKVE IKAAAGSGGS GFVPVFLPAK PTTTPAPRPP |
| | | TPAPTIASQP LSLRPEACRP AAGGAVHTRG LDFACDIYIW APLAGTCGVL |
| | | LLSLVITLYC NHRNRSKRSR LLHSDYMNMT PRRPGPTRKH YQPYAPPRDF |
| | | AAYRSKKVAK KPTNKAPHPK QEPQEINFPD DLPGSNTAAP VQETLHGCQP |
| | | VTQEDGKESR ISVQERQ |
| 139 | CD8a_HuT84.66_ | MALPVTALLL PLALLLHAAR PEVQLVESGG GLVQPGGSLR LSCAASGFNI |
| | spCD8_ | KDTYMHWVRQ APGKGLEWVA RIDPANGNSK YADSVKGRFT ISADTSKNTA |
| | CD28_CD40 | YLQMNSLRAE DTAVYYCAPF GYYVSDYAMA YWGQGTLVTV SSGGGGSGGG |
| | | GSGGGGSDIQ LTQSPSSLSA SVGDRVTITC RAGESVDIFG VGFLHWYQQK |
| | | PGKAPKLLIY RASNLESGVP SRFSGSGSRT DFTLTISSLQ PEDFATYYCQ |
| | | QTNEDPYTFG QGTKVEIKAA AGSGGSGFVP VFLPAKPTTT PAPRPPTPAP |
| | | TIASQPLSLR PEACRPAAGG AVHTRGLDFA CDIYIWAPLA GTCGVLLLSL |
| | | VITLYCNHRN RSKRSRLLHS DYMNMTPRRP GPTRKHYQPY APPRDFAAYR |
| | | SKKVAKKPTN KAPHPKQEPQ EINFPDDLPG SNTAAPVQET LHGCQPVTQE |
| | | DGKESRISVQ ERQ |
| 140 | CD2_HuT84.66_ | MSFPCKFVAS FLLIFNVSSK GAVSEVQLVE SGGGLVQPGG SLRLSCAASG |
| | spCD8_ | FNIKDTYMHW VRQAPGKGLE WVARIDPANG NSKYADSVKG RFTISADTSK |
| | CD28_CD40 | NTAYLQMNSL RAEDTAVYYC APFGYYVSDY AMAYWGQGTL VTVSSGGGGS |
| | | GGGGSGGGGS DIQLTQSPSS LSASVGDRVT ITCRAGESVD IFGVGFLHWY |
| | | QQKPGKAPKL LIYRASNLES GVPSRFSGSG SRTDFTLTIS SLQPEDFATY |
| | | YCQQTNEDPY TFGQGTKVEI KAAAGSGGSG FVPVFLPAKP TTTPAPRPPT |
| | | PAPTIASQPL SLRPEACRPA AGGAVHTRGL DFACDIYIWA PLAGTCGVLL |
| | | LSLVITLYCN HRNRSKRSRL LHSDYMNMTP RRPGPTRKHY QPYAPPRDFA |
| | | AYRSKKVAKK PTNKAPHPKQ EPQEINFPDD LPGSNTAAPV QETLHGCQPV |
| | | TQEDGKESRI SVQERQ |
| 141 | IL2_HuT84.66_ | MYRMQLLSCI ALSLALVTNS EVQLVESGGG LVQPGGSLRL SCAASGFNIK |
| | spCD8_ | DTYMHWVRQA PGKGLEWVAR IDPANGNSKY ADSVKGRFTI SADTSKNTAY |
| | CD28_CD40 | LQMNSLRAED TAVYYCAPFG YYVSDYAMAY WGQGTLVTVS SGGGGSGGGG |
| | | SGGGGSDIQL TQSPSSLSAS VGDRVTITCR AGESVDIFGV GFLHWYQQKP |
| | | GKAPKLLIYR ASNLESGVPS RFSGSGSRTD FTLTISSLQP EDFATYYCQQ |
| | | TNEDPYTFGQ GTKVEIKAAA GSGGSGFVPV FLPAKPTTTP APRPPTPAPT |
| | | IASQPLSLRP EACRPAAGGA VHTRGLDFAC DIYIWAPLAG TCGVLLLSLV |
| | | ITLYCNHRNR SKRSRLLHSD YMNMTPRRPG PTRKHYQPYA PPRDFAAYRS |
| | | KKVAKKPTNK APHPKQEPQE INFPDDLPGS NTAAPVQETL HGCQPVTQED |
| | | GKESRISVQE RQ |
| 142 | GM-CSF_ | MWLQSLLLLG TVACSISEVQ LVESGGGLVQ PGGSLRLSCA ASGFNIKDTY |
| | HuT84.66_ | MHWVRQAPGK GLEWVARIDP ANGNSKYADS VKGRFTISAD TSKNTAYLQM |
| | spCD8_CD28_ | NSLRAEDTAV YYCAPFGYYV SDYAMAYWGQ GTLVTVSSGG GGSGGGGSGG |
| | CD40 | GGSDIQLTQS PSSLSASVGD RVTITCRAGE SVDIFGVGFL HWYQQKPGKA |
| | | PKLLIYRASN LESGVPSRFS GSGSRTDFTL TISSLQPEDF ATYYCQQTNE |
| | | DPYTFGQGTK VEIKAAAGSG GSGFVPVFLP AKPTTTPAPR PPTPAPTIAS |
| | | QPLSLRPEAC RPAAGGAVHT RGLDFACDIY IWAPLAGTCG VLLLSLVITL |
| | | YCNHRNRSKR SRLLHSDYMN MTPRRPGPTR KHYQPYAPPR DFAAYRSKKV |
| | | AKKPTNKAPH PKQEPQEINF PDDLPGSNTA APVQETLHGC QPVTQEDGKE |
| | | SRISVQERQ |
| 143 | hIgGk- | MEAPAQLLFL LLLWLPDTTR EVQLVESGGG LVQPGGSLRL SCAASGFNIK |
| | VIII_HuT84.66_ | DTYMHWVRQA PGKGLEWVAR IDPANGNSKY ADSVKGRFTI SADTSKNTAY |
| | spCD8_ | LQMNSLRAED TAVYYCAPFG YYVSDYAMAY WGQGTLVTVS SGGGGSGGGG |
| | CD28_CD40 | SGGGGSDIQL TQSPSSLSAS VGDRVTITCR AGESVDIFGV GFLHWYQQKP |
| | | GKAPKLLIYR ASNLESGVPS RFSGSGSRTD FTLTISSLQP EDFATYYCQQ |
| | | TNEDPYTFGQ GTKVEIKAAA GSGGSGFVPV FLPAKPTTTP APRPPTPAPT |
| | | IASQPLSLRP EACRPAAGGA VHTRGLDFAC DIYIWAPLAG TCGVLLLSLV |
| | | ITLYCNHRNR SKRSRLLHSD YMNMTPRRPG PTRKHYQPYA PPRDFAAYRS |
| | | KKVAKKPTNK APHPKQEPQE INFPDDLPGS NTAAPVQETL HGCQPVTQED |
| | | GKESRISVQE RQ |
| 144 | OSM_HuT84.66_ | MGVLLTQRTL LSLVLALLFP SMASMEVQLV ESGGGLVQPG GSLRLSCAAS |
| | CD28TM_ | GFNIKDTYMH WVRQAPGKGL EWVARIDPAN GNSKYADSVK GRFTISADTS |
| | CD28_CD40 | KNTAYLQMNS LRAEDTAVYY CAPFGYYVSD YAMAYWGQGT LVTVSSGGGG |
| | | SGGGGSGGGG SDIQLTQSPS SLSASVGDRV TITCRAGESV DIFGVGFLHW |
| | | YQQKPGKAPK LLIYRASNLE SGVPSRFSGS GSRTDFTLTI SSLQPEDFAT |
| | | YYCQQTNEDP YTFGQGTKVE IKAAAGSGGS GFWVLVVVGG VLACYSLLVT |
| | | VAFIIFWVRS KRSRLLHSDY MNMTPRRPGP TRKHYQPYAP PRDFAAYRSK |
| | | KVAKKPTNKA PHPKQEPQEI NFPDDLPGSN TAAPVQETLH GCQPVTQEDG |
| | | KESRISVQER Q |
| 145 | CD8a_HuT84.66_ | MALPVTALLL PLALLLHAAR PEVQLVESGG GLVQPGGSLR LSCAASGFNI |
| | CD28TM_ | KDTYMHWVRQ APGKGLEWVA RIDPANGNSK YADSVKGRFT ISADTSKNTA |
| | CD28_CD40 | YLQMNSLRAE DTAVYYCAPF GYYVSDYAMA YWGQGTLVTV SSGGGGSGGG |
| | | GSGGGGSDIQ LTQSPSSLSA SVGDRVTITC RAGESVDIFG VGFLHWYQQK |
| | | PGKAPKLLIY RASNLESGVP SRFSGSGSRT DFTLTISSLQ PEDFATYYCQ |
| | | QTNEDPYTFG QGTKVEIKAA AGSGGSGFWV LVVVGGVLAC YSLLVTVAFI |
| | | IFWVRSKRSR LLHSDYMNMT PRRPGPTRKH YQPYAPPRDF AAYRSKKVAK |
| | | KPTNKAPHPK QEPQEINFPD DLPGSNTAAP VQETLHGCQP VTQEDGKESR |
| | | ISVQERQ |
| 146 | CD2_HuT84.66_ | MSFPCKFVAS FLLIFNVSSK GAVSEVQLVE SGGGLVQPGG SLRLSCAASG |
| | CD28TM_ | FNIKDTYMHW VRQAPGKGLE WVARIDPANG NSKYADSVKG RFTISADTSK |
| | CD28_CD40 | NTAYLQMNSL RAEDTAVYYC APFGYYVSDY AMAYWGQGTL VTVSSGGGGS |
| | | GGGGSGGGGS DIQLTQSPSS LSASVGDRVT ITCRAGESVD IFGVGFLHWY |
| | | QQKPGKAPKL LIYRASNLES GVPSRFSGSG SRTDFTLTIS SLQPEDFATY |
| | | YCQQTNEDPY TFGQGTKVEI KAAAGSGGSG FWVLVVVGGV LACYSLLVTV |
| | | AFIIFWVRSK RSRLLHSDYM NMTPRRPGPT RKHYQPYAPP RDFAAYRSKK |
| | | VAKKPTNKAP HPKQEPQEIN FPDDLPGSNT AAPVQETLHG CQPVTQEDGK |
| | | ESRISVQERQ |
| 147 | IL2_HuT84.66_ | MYRMQLLSCI ALSLALVTNS EVQLVESGGG LVQPGGSLRL SCAASGFNIK |
| | CD28TM_ | DTYMHWVRQA PGKGLEWVAR IDPANGNSKY ADSVKGRFTI SADTSKNTAY |
| | CD28_CD40 | LQMNSLRAED TAVYYCAPFG YYVSDYAMAY WGQGTLVTVS SGGGGSGGGG |
| | | SGGGGSDIQL TQSPSSLSAS VGDRVTITCR AGESVDIFGV GFLHWYQQKP |
| | | GKAPKLLIYR ASNLESGVPS RFSGSGSRTD FTLTISSLQP EDFATYYCQQ |
| | | TNEDPYTFGQ GTKVEIKAAA GSGGSGFWVL VVVGGVLACY SLLVTVAFII |
| | | FWVRSKRSRL LHSDYMNMTP RRPGPTRKHY QPYAPPRDFA AYRSKKVAKK |
| | | PTNKAPHPKQ EPQEINFPDD LPGSNTAAPV QETLHGCQPV TQEDGKESRI |
| | | SVQERQ |
| 148 | GM-CSF_ | MWLQSLLLLG TVACSISEVQ LVESGGGLVQ PGGSLRLSCA ASGFNIKDTY |
| | HuT84.66_ | MHWVRQAPGK GLEWVARIDP ANGNSKYADS VKGRFTISAD TSKNTAYLQM |
| | CD28TM_CD28_ | NSLRAEDTAV YYCAPFGYYV SDYAMAYWGQ GTLVTVSSGG GGSGGGGSGG |
| | CD40 | GGSDIQLTQS PSSLSASVGD RVTITCRAGE SVDIFGVGFL HWYQQKPGKA |
| | | PKLLIYRASN LESGVPSRFS GSGSRTDFTL TISSLQPEDF ATYYCQQTNE |
| | | DPYTFGQGTK VEIKAAAGSG GSGFWVLVVV GGVLACYSLL VTVAFIIFWV |
| | | RSKRSRLLHS DYMNMTPRRP GPTRKHYQPY APPRDFAAYR SKKVAKKPTN |
| | | KAPHPKQEPQ EINFPDDLPG SNTAAPVQET LHGCQPVTQE DGKESRISVQ |
| | | ERQ |
| 149 | hIgGk- | MEAPAQLLFL LLLWLPDTTR EVQLVESGGG LVQPGGSLRL SCAASGFNIK |
| | VIII_HuT84.66_ | DTYMHWVRQA PGKGLEWVAR IDPANGNSKY ADSVKGRFTI SADTSKNTAY |
| | CD28TM_ | LQMNSLRAED TAVYYCAPFG YYVSDYAMAY WGQGTLVTVS SGGGGSGGGG |
| | CD28_CD40 | SGGGGSDIQL TQSPSSLSAS VGDRVTITCR AGESVDIFGV GFLHWYQQKP |
| | | GKAPKLLIYR ASNLESGVPS RFSGSGSRTD FTLTISSLQP EDFATYYCQQ |
| | | TNEDPYTFGQ GTKVEIKAAA GSGGSGFWVL VVVGGVLACY SLLVTVAFII |
| | | FWVRSKRSRL LHSDYMNMTP RRPGPTRKHY QPYAPPRDFA AYRSKKVAKK |
| | | PTNKAPHPKQ EPQEINFPDD LPGSNTAAPV QETLHGCQPV TQEDGKESRI |
| | | SVQERQ |
| 150 | OSM_MFE23_ | MGVLLTQRTL LSLVLALLFP SMASMQVKLQ QSGAELVRSG TSVKLSCTAS |
| | spCD28(trun)_ | GFNIKDSYMH WLRQGPEQGL EWIGWIDPEN GDTEYAPKFQ GKATFTTDTS |
| | CD28_CD40 | SNTAYLQLSS LTSEDTAVYY CNEGTPTGPY YFDYWGQGTT VTVSSGGGGS |
| | | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGIIHV KGKHLCPSPL FPGPSKPFWV |
| | | LVVVGGVLAC YSLLVTVAFI IFWVRSKRSR LLHSDYMNMT PRRPGPTRKH |
| | | YQPYAPPRDF AAYRSKKVAK KPTNKAPHPK QEPQEINFPD DLPGSNTAAP |
| | | VQETLHGCQP VTQEDGKESR ISVQERQ |
| 151 | CD8a_MFE23_ | MALPVTALLL PLALLLHAAR PQVKLQQSGA ELVRSGTSVK LSCTASGFNI |
| | spCD28(trun)_ | KDSYMHWLRQ GPEQGLEWIG WIDPENGDTE YAPKFQGKAT FTTDTSSNTA |
| | CD28_CD40 | YLQLSSLTSE DTAVYYCNEG TPTGPYYFDY WGQGTTVTVS SGGGGSGGGG |
| | | SGGGGSENVL TQSPAIMSAS PGEKVTITCS ASSSVSYMHW FQQKPGTSPK |
| | | LWIYSTSNLA SGVPARFSGS GSGTSYSLTI SRMEAEDAAT YYCQQRSSYP |
| | | LTFGAGTKLE LKRAAAGSGG SGIIHVKGKH LCPSPLFPGP SKPFWVLVVV |
| | | GGVLACYSLL VTVAFIIFWV RSKRSRLLHS DYMNMTPRRP GPTRKHYQPY |
| | | APPRDFAAYR SKKVAKKPTN KAPHPKQEPQ EINFPDDLPG SNTAAPVQET |
| | | LHGCQPVTQE DGKESRISVQ ERQ |
| 152 | CD2_MFE23_ | MSFPCKFVAS FLLIFNVSSK GAVSQVKLQQ SGAELVRSGT SVKLSCTASG |
| | spCD28(trun)_ | FNIKDSYMHW LRQGPEQGLE WIGWIDPENG DTEYAPKFQG KATFTTDTSS |
| | CD28_CD40 | NTAYLQLSSL TSEDTAVYYC NEGTPTGPYY FDYWGQGTTV TVSSGGGGSG |
| | | GGGSGGGGSE NVLTQSPAIM SASPGEKVTI TCSASSSVSY MHWFQQKPGT |
| | | SPKLWIYSTS NLASGVPARF SGSGSGTSYS LTISRMEAED AATYYCQQRS |
| | | SYPLTFGAGT KLELKRAAAG SGGSGIIHVK GKHLCPSPLF PGPSKPFWVL |
| | | VVVGGVLACY SLLVTVAFII FWVRSKRSRL LHSDYMNMTP RRPGPTRKHY |
| | | QPYAPPRDFA AYRSKKVAKK PTNKAPHPKQ EPQEINFPDD LPGSNTAAPV |
| | | QETLHGCQPV TQEDGKESRI SVQERQ |
| 153 | IL2_MFE23_ | MYRMQLLSCI ALSLALVTNS QVKLQQSGAE LVRSGTSVKL SCTASGFNIK |
| | spCD28(trun)_ | DSYMHWLRQG PEQGLEWIGW IDPENGDTEY APKFQGKATF TTDTSSNTAY |
| | CD28_CD40 | LQLSSLTSED TAVYYCNEGT PTGPYYFDYW GQGTTVTVSS GGGGSGGGGS |
| | | GGGGSENVLT QSPAIMSASP GEKVTITCSA SSSVSYMHWF QQKPGTSPKL |
| | | WIYSTSNLAS GVPARFSGSG SGTSYSLTIS RMEAEDAATY YCQQRSSYPL |
| | | TFGAGTKLEL KRAAAGSGGS GIIHVKGKHL CPSPLFPGPS KPFWVLVVVG |
| | | GVLACYSLLV TVAFIIFWVR SKRSRLLHSD YMNMTPRRPG PTRKHYQPYA |
| | | PPRDFAAYRS KKVAKKPTNK APHPKQEPQE INFPDDLPGS NTAAPVQETL |
| | | HGCQPVTQED GKESRISVQE RQ |
| 154 | GM-CSF_MFE23_ | MWLQSLLLLG TVACSISQVK LQQSGAELVR SGTSVKLSCT ASGFNIKDSY |
| | spCD28(trun)_ | MHWLRQGPEQ GLEWIGWIDP ENGDTEYAPK FQGKATFTTD TSSNTAYLQL |
| | CD28_CD40 | SSLTSEDTAV YYCNEGTPTG PYYFDYWGQG TTVTVSSGGG GSGGGGSGGG |
| | | GSENVLTQSP AIMSASPGEK VTITCSASSS VSYMHWFQQK PGTSPKLWIY |
| | | STSNLASGVP ARFSGSGSGT SYSLTISRME AEDAATYYCQ QRSSYPLTFG |
| | | AGTKLELKRA AAGSGGSGII HVKGKHLCPS PLFPGPSKPF WVLVVVGGVL |
| | | ACYSLLVTVA FIIFWVRSKR SRLLHSDYMN MTPRRPGPTR KHYQPYAPPR |
| | | DFAAYRSKKV AKKPTNKAPH PKQEPQEINF PDDLPGSNTA APVQETLHGC |
| | | QPVTQEDGKE SRISVQERQ |
| 155 | hIgGk- | MEAPAQLLFL LLLWLPDTTR QVKLQQSGAE LVRSGTSVKL SCTASGFNIK |
| | VIII_MFE23_ | DSYMHWLRQG PEQGLEWIGW IDPENGDTEY APKFQGKATF TTDTSSNTAY |
| | spCD28(trun)_ | LQLSSLTSED TAVYYCNEGT PTGPYYFDYW GQGTTVTVSS GGGGSGGGGS |
| | CD28_CD40 | GGGGSENVLT QSPAIMSASP GEKVTITCSA SSSVSYMHWF QQKPGTSPKL |
| | | WIYSTSNLAS GVPARFSGSG SGTSYSLTIS RMEAEDAATY YCQQRSSYPL |
| | | TFGAGTKLEL KRAAAGSGGS GIIHVKGKHL CPSPLFPGPS KPFWVLVVVG |
| | | GVLACYSLLV TVAFIIFWVR SKRSRLLHSD YMNMTPRRPG PTRKHYQPYA |
| | | PPRDFAAYRS KKVAKKPTNK APHPKQEPQE INFPDDLPGS NTAAPVQETL |
| | | HGCQPVTQED GKESRISVQE RQ |
| 156 | OSM_MFE23 | MGVLLTQRTL LSLVLALLFP SMASMQVOLQ QSGAELVRSG TSVKLSCTAS |
| | (K > Q)_spCD28 | GFNIKDSYMH WLRQGPEQGL EWIGWIDPEN GDTEYAPKFQ GKATFTTDTS |
| | (trun)_ | SNTAYLQLSS LTSEDTAVYY CNEGTPTGPY YFDYWGQGTT VTVSSGGGGS |
| | CD28_CD40 | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGIIHV KGKHLCPSPL FPGPSKPFWV |
| | | LVVVGGVLAC YSLLVTVAFI IFWVRSKRSR LLHSDYMNMT PRRPGPTRKH |
| | | YQPYAPPRDF AAYRSKKVAK KPTNKAPHPK QEPQEINFPD DLPGSNTAAP |
| | | VQETLHGCQP VTQEDGKESR ISVQERQ |
| 157 | CD8a_ | MALPVTALLL PLALLLHAAR PQVQLQQSGA ELVRSGTSVK LSCTASGFNI |
| | MFE23(K > Q)_ | KDSYMHWLRQ GPEQGLEWIG WIDPENGDTE YAPKFQGKAT FTTDTSSNTA |
| | spCD28(trun)_ | YLQLSSLTSE DTAVYYCNEG TPTGPYYFDY WGQGTTVTVS SGGGGSGGGG |
| | CD28_CD40 | SGGGGSENVL TQSPAIMSAS PGEKVTITCS ASSSVSYMHW FQQKPGTSPK |
| | | LWIYSTSNLA SGVPARFSGS GSGTSYSLTI SRMEAEDAAT YYCQQRSSYP |
| | | LTFGAGTKLE LKRAAAGSGG SGIIHVKGKH LCPSPLFPGP SKPFWVLVVV |
| | | GGVLACYSLL VTVAFIIFWV RSKRSRLLHS DYMNMTPRRP GPTRKHYQPY |
| | | APPRDFAAYR SKKVAKKPTN KAPHPKQEPQ EINFPDDLPG SNTAAPVQET |
| | | LHGCQPVTQE DGKESRISVQ ERQ |
| 158 | CD2_MFE23 | MSFPCKFVAS FLLIFNVSSK GAVSQVQLQQ SGAELVRSGT SVKLSCTASG |
| | (K > Q)_spCD28 | FNIKDSYMHW LRQGPEQGLE WIGWIDPENG DTEYAPKFQG KATFTTDTSS |
| | (trun)_ | NTAYLQLSSL TSEDTAVYYC NEGTPTGPYY FDYWGQGTTV TVSSGGGGSG |
| | CD28_CD40 | GGGSGGGGSE NVLTQSPAIM SASPGEKVTI TCSASSSVSY MHWFQQKPGT |
| | | SPKLWIYSTS NLASGVPARF SGSGSGTSYS LTISRMEAED AATYYCQQRS |
| | | SYPLTFGAGT KLELKRAAAG SGGSGIIHVK GKHLCPSPLF PGPSKPFWVL |
| | | VVVGGVLACY SLLVTVAFII FWVRSKRSRL LHSDYMNMTP RRPGPTRKHY |
| | | QPYAPPRDFA AYRSKKVAKK PTNKAPHPKQ EPQEINFPDD LPGSNTAAPV |
| | | QETLHGCQPV TQEDGKESRI SVQERQ |
| 159 | IL2_MFE23 | MYRMQLLSCI ALSLALVTNS QVQLQQSGAE LVRSGTSVKL SCTASGFNIK |
| | (K > Q)_spCD28 | DSYMHWLRQG PEQGLEWIGW IDPENGDTEY APKFQGKATF TTDTSSNTAY |
| | (trun)_ | LQLSSLTSED TAVYYCNEGT PTGPYYFDYW GQGTTVTVSS GGGGSGGGGS |
| | CD28_CD40 | GGGGSENVLT QSPAIMSASP GEKVTITCSA SSSVSYMHWF QQKPGTSPKL |
| | | WIYSTSNLAS GVPARFSGSG SGTSYSLTIS RMEAEDAATY YCQQRSSYPL |
| | | TFGAGTKLEL KRAAAGSGGS GIIHVKGKHL CPSPLFPGPS KPFWVLVVVG |
| | | GVLACYSLLV TVAFIIFWVR SKRSRLLHSD YMNMTPRRPG PTRKHYQPYA |
| | | PPRDFAAYRS KKVAKKPTNK APHPKQEPQE INFPDDLPGS NTAAPVQETL |
| | | HGCQPVTQED GKESRISVQE RQ |
| 160 | GM-CSF_ | MWLQSLLLLG TVACSISQVQ LQQSGAELVR SGTSVKLSCT ASGFNIKDSY |
| | MFE23(K > Q)_ | MHWLRQGPEQ GLEWIGWIDP ENGDTEYAPK FQGKATFTTD TSSNTAYLQL |
| | spCD28(trun)_ | SSLTSEDTAV YYCNEGTPTG PYYFDYWGQG TTVTVSSGGG GSGGGGSGGG |
| | CD28_CD40 | GSENVLTQSP AIMSASPGEK VTITCSASSS VSYMHWFQQK PGTSPKLWIY |
| | | STSNLASGVP ARFSGSGSGT SYSLTISRME AEDAATYYCQ QRSSYPLTFG |
| | | AGTKLELKRA AAGSGGSGII HVKGKHLCPS PLFPGPSKPF WVLVVVGGVL |
| | | ACYSLLVTVA FIIFWVRSKR SRLLHSDYMN MTPRRPGPTR KHYQPYAPPR |
| | | DFAAYRSKKV AKKPTNKAPH PKQEPQEINF PDDLPGSNTA APVQETLHGC |
| | | QPVTQEDGKE SRISVQERQ |
| 161 | hIgGk-VIII_ | MEAPAQLLFL LLLWLPDTTR QVQLQQSGAE LVRSGTSVKL SCTASGFNIK |
| | MFE23(K > Q)_ | DSYMHWLRQG PEQGLEWIGW IDPENGDTEY APKFQGKATF TTDTSSNTAY |
| | spCD28(trun)_ | LQLSSLTSED TAVYYCNEGT PTGPYYFDYW GQGTTVTVSS GGGGSGGGGS |
| | CD28_CD40 | GGGGSENVLT QSPAIMSASP GEKVTITCSA SSSVSYMHWF QQKPGTSPKL |
| | | WIYSTSNLAS GVPARFSGSG SGTSYSLTIS RMEAEDAATY YCQQRSSYPL |
| | | TFGAGTKLEL KRAAAGSGGS GIIHVKGKHL CPSPLFPGPS KPFWVLVVVG |
| | | GVLACYSLLV TVAFIIFWVR SKRSRLLHSD YMNMTPRRPG PTRKHYQPYA |
| | | PPRDFAAYRS KKVAKKPTNK APHPKQEPQE INFPDDLPGS NTAAPVQETL |
| | | HGCQPVTQED GKESRISVQE RQ |
| 162 | OSM_HuMFE23_ | MGVLLTQRTL LSLVLALLFP SMASMQVKLE QSGAEVVKPG ASVKLSCKAS |
| | spCD28(trun)_ | GFNIKDSYMH WLRQGPGQRL EWIGWIDPEN GDTEYAPKFQ GKATFTTDTS |
| | CD28_CD40 | ANTAYLGLSS LRPEDTAVYY CNEGTPTGPY YFDYWGQGTL VTVSSGGGGS |
| | CTP244 | GGGGSGGGGS ENVLTQSPSS MSASVGDRVN IACSASSSVS YMHWFQQKPG |
| | | KSPKLWIYST SNLASGVPSR FSGSGSGTDY SLTISSMQPE DAATYYCQQR |
| | | SSYPLTFGGG TKLEIKAAAG SGGSGIIHVK GKHLCPSPLF PGPSKPFWVL |
| | | VVVGGVLACY SLLVTVAFII FWVRSKRSRL LHSDYMNMTP RRPGPTRKHY |
| | | QPYAPPRDFA AYRSKKVAKK PTNKAPHPKQ EPQEINFPDD LPGSNTAAPV |
| | | QETLHGCQPV TQEDGKESRI SVQERQ |
| 163 | CD8a_HuMFE23_ | MALPVTALLL PLALLLHAAR PQVKLEQSGA EVVKPGASVK LSCKASGFNI |
| | spCD28(trun)_ | KDSYMHWLRQ GPGQRLEWIG WIDPENGDTE YAPKFQGKAT FTTDTSANTA |
| | CD28_CD40 | YLGLSSLRPE DTAVYYCNEG TPTGPYYFDY WGQGTLVTVS SGGGGSGGGG |
| | | SGGGGSENVL TQSPSSMSAS VGDRVNIACS ASSSVSYMHW FQQKPGKSPK |
| | | LWIYSTSNLA SGVPSRFSGS GSGTDYSLTI SSMQPEDAAT YYCQQRSSYP |
| | | LTFGGGTKLE IKAAAGSGGS GIIHVKGKHL CPSPLFPGPS KPFWVLVVVG |
| | | GVLACYSLLV TVAFIIFWVR SKRSRLLHSD YMNMTPRRPG PTRKHYQPYA |
| | | PPRDFAAYRS KKVAKKPTNK APHPKQEPQE INFPDDLPGS NTAAPVQETL |
| | | HGCQPVTQED GKESRISVQE RQ |
| 164 | CD2_HuMFE23_ | MSFPCKFVAS FLLIFNVSSK GAVSQVKLEQ SGAEVVKPGA SVKLSCKASG |
| | spCD28(trun)_ | FNIKDSYMHW LRQGPGQRLE WIGWIDPENG DTEYAPKFQG KATFTTDTSA |
| | CD28_CD40 | NTAYLGLSSL RPEDTAVYYC NEGTPTGPYY FDYWGQGTLV TVSSGGGGSG |
| | | GGGSGGGGSE NVLTQSPSSM SASVGDRVNI ACSASSSVSY MHWFQQKPGK |
| | | SPKLWIYSTS NLASGVPSRF SGSGSGTDYS LTISSMQPED AATYYCQQRS |
| | | SYPLTFGGGT KLEIKAAAGS GGSGIIHVKG KHLCPSPLFP GPSKPFWVLV |
| | | VVGGVLACYS LLVTVAFIIF WVRSKRSRLL HSDYMNMTPR RPGPTRKHYQ |
| | | PYAPPRDFAA YRSKKVAKKP TNKAPHPKQE PQEINFPDDL PGSNTAAPVQ |
| | | ETLHGCQPVT QEDGKESRIS VQERQ |
| 165 | IL2_HuMFE23_ | MYRMQLLSCI ALSLALVTNS QVKLEQSGAE VVKPGASVKL SCKASGFNIK |
| | spCD28(trun)_ | DSYMHWLRQG PGQRLEWIGW IDPENGDTEY APKFQGKATF TTDTSANTAY |
| | CD28_CD40 | LGLSSLRPED TAVYYCNEGT PTGPYYFDYW GQGTLVTVSS GGGGSGGGGS |
| | | GGGGSENVLT QSPSSMSASV GDRVNIACSA SSSVSYMHWF QQKPGKSPKL |
| | | WIYSTSNLAS GVPSRFSGSG SGTDYSLTIS SMQPEDAATY YCQQRSSYPL |
| | | TFGGGTKLEI KAAAGSGGSG IIHVKGKHLC PSPLFPGPSK PFWVLVVVGG |
| | | VLACYSLLVT VAFIIFWVRS KRSRLLHSDY MNMTPRRPGP TRKHYQPYAP |
| | | PRDFAAYRSK KVAKKPTNKA PHPKQEPQEI NFPDDLPGSN TAAPVQETLH |
| | | GCQPVTQEDG KESRISVQER Q |
| 166 | GM-CSF_HuMFE23_ | MWLQSLLLLG TVACSISQVK LEQSGAEVVK PGASVKLSCK ASGFNIKDSY |
| | spCD28(trun)_ | MHWLRQGPGQ RLEWIGWIDP ENGDTEYAPK FQGKATFTTD TSANTAYLGL |
| | CD28_CD40 | SSLRPEDTAV YYCNEGTPTG PYYFDYWGQG TLVTVSSGGG GSGGGGSGGG |
| | | GSENVLTQSP SSMSASVGDR VNIACSASSS VSYMHWFQQK PGKSPKLWIY |
| | | STSNLASGVP SRFSGSGSGT DYSLTISSMQ PEDAATYYCQ QRSSYPLTFG |
| | | GGTKLEIKAA AGSGGSGIIH VKGKHLCPSP LFPGPSKPFW VLVVVGGVLA |
| | | CYSLLVTVAF IIFWVRSKRS RLLHSDYMNM TPRRPGPTRK HYQPYAPPRD |
| | | FAAYRSKKVA KKPTNKAPHP KQEPQEINFP DDLPGSNTAA PVQETLHGCQ |
| | | PVTQEDGKES RISVQERQ |
| 167 | hIgGk-VIII_ | MEAPAQLLFL LLLWLPDTTR QVKLEQSGAE VVKPGASVKL SCKASGFNIK |
| | HuMFE23_ | DSYMHWLRQG PGQRLEWIGW IDPENGDTEY APKFQGKATF TTDTSANTAY |
| | spCD28(trun)_ | LGLSSLRPED TAVYYCNEGT PTGPYYFDYW GQGTLVTVSS GGGGSGGGGS |
| | CD28_CD40 | GGGGSENVLT QSPSSMSASV GDRVNIACSA SSSVSYMHWF QQKPGKSPKL |
| | | WIYSTSNLAS GVPSRFSGSG SGTDYSLTIS SMQPEDAATY YCQQRSSYPL |
| | | TFGGGTKLEI KAAAGSGGSG IIHVKGKHLC PSPLFPGPSK PFWVLVVVGG |
| | | VLACYSLLVT VAFIIFWVRS KRSRLLHSDY MNMTPRRPGP TRKHYQPYAP |
| | | PRDFAAYRSK KVAKKPTNKA PHPKQEPQEI NFPDDLPGSN TAAPVQETLH |
| | | GCQPVTQEDG KESRISVQER Q |
| 168 | OSM_CEA6_ | MGVLLTQRTL LSLVLALLFP SMASMQVQLV QSGAEVKKPG SSVKVSCKAS |
| | spCD28(trun)_ | GGTFSNSPIN WLRQAPGQGL EWMGSIIPSF GTANYAQKFQ GRLTITADES |
| | CD28_CD40 | TSTAYMELSS LRSEDTAVYY CAGRSHNYEL YYYYMDVWGQ GTMVTVSSGG |
| | | GGSGGGGSGG GGSDIQMTQS PSTLSASIGD RVTITCRASE GIYHWLAWYQ |
| | | QKPGKAPKLL IYKASSLASG APSRFSGSGS GTDFTLTISS LQPDDFATYY |
| | | CQQYSNYPLT FGGGTKLEIK RAAAGSGGSG IIHVKGKHLC PSPLFPGPSK |
| | | PFWVLVVVGG VLACYSLLVT VAFIIFWVRS KRSRLLHSDY MNMTPRRPGP |
| | | TRKHYQPYAP PRDFAAYRSK KVAKKPTNKA PHPKQEPQEI NFPDDLPGSN |
| | | TAAPVQETLH GCQPVTQEDG KESRISVQER Q |
| 169 | CD8a_CEA6_ | MALPVTALLL PLALLLHAAR PQVQLVQSGA EVKKPGSSVK VSCKASGGTF |
| | spCD28(trun)_ | SNSPINWLRQ APGQGLEWMG SIIPSFGTAN YAQKFQGRLT ITADESTSTA |
| | CD28_CD40 | YMELSSLRSE DTAVYYCAGR SHNYELYYYY MDVWGQGTMV TVSSGGGGSG |
| | | GGGSGGGGSD IQMTQSPSTL SASIGDRVTI TCRASEGIYH WLAWYQQKPG |
| | | KAPKLLIYKA SSLASGAPSR FSGSGSGTDF TLTISSLQPD DFATYYCQQY |
| | | SNYPLTFGGG TKLEIKRAAA GSGGSGIIHV KGKHLCPSPL FPGPSKPFWV |
| | | LVVVGGVLAC YSLLVTVAFI IFWVRSKRSR LLHSDYMNMT PRRPGPTRKH |
| | | YQPYAPPRDF AAYRSKKVAK KPTNKAPHPK QEPQEINFPD DLPGSNTAAP |
| | | VQETLHGCQP VTQEDGKESR ISVQERQ |
| 170 | CD2_CEA6_ | MSFPCKFVAS FLLIFNVSSK GAVSQVQLVQ SGAEVKKPGS SVKVSCKASG |
| | spCD28(trun)_ | GTFSNSPINW LRQAPGQGLE WMGSIIPSFG TANYAQKFQG RLTITADEST |
| | CD28_CD40 | STAYMELSSL RSEDTAVYYC AGRSHNYELY YYYMDVWGQG TMVTVSSGGG |
| | | GSGGGGSGGG GSDIQMTQSP STLSASIGDR VTITCRASEG IYHWLAWYQQ |
| | | KPGKAPKLLI YKASSLASGA PSRFSGSGSG TDFTLTISSL QPDDFATYYC |
| | | QQYSNYPLTF GGGTKLEIKR AAAGSGGSGI IHVKGKHLCP SPLFPGPSKP |
| | | FWVLVVVGGV LACYSLLVTV AFIIFWVRSK RSRLLHSDYM NMTPRRPGPT |
| | | RKHYQPYAPP RDFAAYRSKK VAKKPTNKAP HPKQEPQEIN FPDDLPGSNT |
| | | AAPVQETLHG CQPVTQEDGK ESRISVQERQ |
| 171 | IL2_CEA6_ | MYRMQLLSCI ALSLALVTNS QVQLVQSGAE VKKPGSSVKV SCKASGGTFS |
| | spCD28(trun)_ | NSPINWLRQA PGQGLEWMGS IIPSEGTANY AQKFQGRLTI TADESTSTAY |
| | CD28_CD40 | MELSSLRSED TAVYYCAGRS HNYELYYYYM DVWGQGTMVT VSSGGGGSGG |
| | | GGSGGGGSDI QMTQSPSTLS ASIGDRVTIT CRASEGIYHW LAWYQQKPGK |
| | | APKLLIYKAS SLASGAPSRF SGSGSGTDFT LTISSLQPDD FATYYCQQYS |
| | | NYPLTFGGGT KLEIKRAAAG SGGSGIIHVK GKHLCPSPLF PGPSKPFWVL |
| | | VVVGGVLACY SLLVTVAFII FWVRSKRSRL LHSDYMNMTP RRPGPTRKHY |
| | | QPYAPPRDFA AYRSKKVAKK PTNKAPHPKQ EPQEINFPDD LPGSNTAAPV |
| | | QETLHGCQPV TQEDGKESRI SVQERQ |
| 172 | GM-CSF_CEA6_ | MWLQSLLLLG TVACSISQVQ LVQSGAEVKK PGSSVKVSCK ASGGTFSNSP |
| | spCD28(trun)_ | INWLRQAPGQ GLEWMGSIIP SFGTANYAQK FQGRLTITAD ESTSTAYMEL |
| | CD28_CD40 | SSLRSEDTAV YYCAGRSHNY ELYYYYMDVW GQGTMVTVSS GGGGSGGGGS |
| | | GGGGSDIQMT QSPSTLSASI GDRVTITCRA SEGIYHWLAW YQQKPGKAPK |
| | | LLIYKASSLA SGAPSRFSGS GSGTDFTLTI SSLQPDDFAT YYCQQYSNYP |
| | | LTFGGGTKLE IKRAAAGSGG SGIIHVKGKH LCPSPLFPGP SKPFWVLVVV |
| | | GGVLACYSLL VTVAFIIFWV RSKRSRLLHS DYMNMTPRRP GPTRKHYQPY |
| | | APPRDFAAYR SKKVAKKPTN KAPHPKQEPQ EINFPDDLPG SNTAAPVQET |
| | | LHGCQPVTQE DGKESRISVQ ERQ |
| 173 | hIgGk-VIII_ | MEAPAQLLFL LLLWLPDTTR QVQLVQSGAE VKKPGSSVKV SCKASGGTFS |
| | CEA6_ | NSPINWLRQA PGQGLEWMGS IIPSEGTANY AQKFQGRLTI TADESTSTAY |
| | spCD28(trun)_ | MELSSLRSED TAVYYCAGRS HNYELYYYYM DVWGQGTMVT VSSGGGGSGG |
| | CD28_CD40 | GGSGGGGSDI QMTQSPSTLS ASIGDRVTIT CRASEGIYHW LAWYQQKPGK |
| | | APKLLIYKAS SLASGAPSRF SGSGSGTDFT LTISSLQPDD FATYYCQQYS |
| | | NYPLTFGGGT KLEIKRAAAG SGGSGIIHVK GKHLCPSPLF PGPSKPFWVL |
| | | VVVGGVLACY SLLVTVAFII FWVRSKRSRL LHSDYMNMTP RRPGPTRKHY |
| | | QPYAPPRDFA AYRSKKVAKK PTNKAPHPKQ EPQEINFPDD LPGSNTAAPV |
| | | QETLHGCQPV TQEDGKESRI SVQERQ |
| 174 | OSM_BW431/26_ | MGVLLTQRTL LSLVLALLFP SMASMQLQES GPGLVRPSQT LSLTCTVSGF |
| | spCD28(trun)_ | TISSGYSWHW VRQPPGRGLE WIGYIQYSGI TNYNPSLKSR VTMLVDTSKN |
| | CD28_CD40 | QFSLRLSSVT AADTAVYYCA REDYDYHWYF DVWGQGSLVT VSSGGGGSGG |
| | | GGSGGGGSGV HSDIQMTQSP SSLSASVGDR VTITCSTSSS VSYMHWYQQK |
| | | PGKAPKLLIY STSNLASGVP SRFSGSGSGT DFTFTISSLQ PEDIATYYCH |
| | | QWSSYPTFGQ GTKVEIKRAA AGSGGSGIIH VKGKHLCPSP LFPGPSKPFW |
| | | VLVVVGGVLA CYSLLVTVAF IIFWVRSKRS RLLHSDYMNM TPRRPGPTRK |
| | | HYQPYAPPRD FAAYRSKKVA KKPTNKAPHP KQEPQEINFP DDLPGSNTAA |
| | | PVQETLHGCQ PVTQEDGKES RISVQERQ |
| 175 | CD8a_BW431/26_ | MALPVTALLL PLALLLHAAR PQLQESGPGL VRPSQTLSLT CTVSGFTISS |
| | spCD28(trun)_ | GYSWHWVRQP PGRGLEWIGY IQYSGITNYN PSLKSRVTML VDTSKNQFSL |
| | CD28_CD40 | RLSSVTAADT AVYYCAREDY DYHWYFDVWG QGSLVTVSSG GGGSGGGGSG |
| | | GGGSGVHSDI QMTQSPSSLS ASVGDRVTIT CSTSSSVSYM HWYQQKPGKA |
| | | PKLLIYSTSN LASGVPSRFS GSGSGTDFTF TISSLQPEDI ATYYCHQWSS |
| | | YPTFGQGTKV EIKRAAAGSG GSGIIHVKGK HLCPSPLFPG PSKPFWVLVV |
| | | VGGVLACYSL LVTVAFIIFW VRSKRSRLLH SDYMNMTPRR PGPTRKHYQP |
| | | YAPPRDFAAY RSKKVAKKPT NKAPHPKQEP QEINFPDDLP GSNTAAPVQE |
| | | TLHGCQPVTQ EDGKESRISV QERQ |
| 176 | CD2_BW431/26_ | MSFPCKFVAS FLLIFNVSSK GAVSQLQESG PGLVRPSQTL SLTCTVSGFT |
| | spCD28(trun)_ | ISSGYSWHWV RQPPGRGLEW IGYIQYSGIT NYNPSLKSRV TMLVDTSKNQ |
| | CD28_CD40 | FSLRLSSVTA ADTAVYYCAR EDYDYHWYFD VWGQGSLVTV SSGGGGSGGG |
| | | GSGGGGSGVH SDIQMTQSPS SLSASVGDRV TITCSTSSSV SYMHWYQQKP |
| | | GKAPKLLIYS TSNLASGVPS RFSGSGSGTD FTFTISSLQP EDIATYYCHQ |
| | | WSSYPTFGQG TKVEIKRAAA GSGGSGIIHV KGKHLCPSPL FPGPSKPFWV |
| | | LVVVGGVLAC YSLLVTVAFI IFWVRSKRSR LLHSDYMNMT PRRPGPTRKH |
| | | YQPYAPPRDF AAYRSKKVAK KPTNKAPHPK QEPQEINFPD DLPGSNTAAP |
| | | VQETLHGCQP VTQEDGKESR ISVQERQ |
| 177 | IL2_BW431/26_ | MYRMQLLSCI ALSLALVTNS QLQESGPGLV RPSQTLSLTC TVSGFTISSG |
| | spCD28(trun)_ | YSWHWVRQPP GRGLEWIGYI QYSGITNYNP SLKSRVTMLV DTSKNQFSLR |
| | CD28_CD40 | LSSVTAADTA VYYCAREDYD YHWYFDVWGQ GSLVTVSSGG GGSGGGGSGG |
| | | GGSGVHSDIQ MTQSPSSLSA SVGDRVTITC STSSSVSYMH WYQQKPGKAP |
| | | KLLIYSTSNL ASGVPSRFSG SGSGTDFTFT ISSLQPEDIA TYYCHQWSSY |
| | | PTFGQGTKVE IKRAAAGSGG SGIIHVKGKH LCPSPLFPGP SKPFWVLVVV |
| | | GGVLACYSLL VTVAFIIFWV RSKRSRLLHS DYMNMTPRRP GPTRKHYQPY |
| | | APPRDFAAYR SKKVAKKPTN KAPHPKQEPQ EINFPDDLPG SNTAAPVQET |
| | | LHGCQPVTQE DGKESRISVQ ERQ |
| 178 | GM-CSF_ | MWLQSLLLLG TVACSISQLQ ESGPGLVRPS QTLSLTCTVS GFTISSGYSW |
| | BW431/26_ | HWVRQPPGRG LEWIGYIQYS GITNYNPSLK SRVTMLVDTS KNQFSLRLSS |
| | spCD28(trun)_ | VTAADTAVYY CAREDYDYHW YFDVWGQGSL VTVSSGGGGS GGGGSGGGGS |
| | CD28_CD40 | GVHSDIQMTQ SPSSLSASVG DRVTITCSTS SSVSYMHWYQ QKPGKAPKLL |
| | | IYSTSNLASG VPSRFSGSGS GTDFTFTISS LQPEDIATYY CHQWSSYPTF |
| | | GQGTKVEIKR AAAGSGGSGI IHVKGKHLCP SPLFPGPSKP FWVLVVVGGV |
| | | LACYSLLVTV AFIIFWVRSK RSRLLHSDYM NMTPRRPGPT RKHYQPYAPP |
| | | RDFAAYRSKK VAKKPTNKAP HPKQEPQEIN FPDDLPGSNT AAPVQETLHG |
| | | CQPVTQEDGK ESRISVQERQ |
| 179 | hIgGk-VIII_ | MEAPAQLLFL LLLWLPDTTR QLQESGPGLV RPSQTLSLTC TVSGFTISSG |
| | BW431/26_ | YSWHWVRQPP GRGLEWIGYI QYSGITNYNP SLKSRVTMLV DTSKNQFSLR |
| | spCD28(trun)_ | LSSVTAADTA VYYCAREDYD YHWYFDVWGQ GSLVTVSSGG GGSGGGGSGG |
| | CD28_CD40 | GGSGVHSDIQ MTQSPSSLSA SVGDRVTITC STSSSVSYMH WYQQKPGKAP |
| | | KLLIYSTSNL ASGVPSRFSG SGSGTDFTFT ISSLQPEDIA TYYCHQWSSY |
| | | PTFGQGTKVE IKRAAAGSGG SGIIHVKGKH LCPSPLFPGP SKPFWVLVVV |
| | | GGVLACYSLL VTVAFIIFWV RSKRSRLLHS DYMNMTPRRP GPTRKHYQPY |
| | | APPRDFAAYR SKKVAKKPTN KAPHPKQEPQ EINFPDDLPG SNTAAPVQET |
| | | LHGCQPVTQE DGKESRISVQ ERQ |
| 180 | OSM_HuT84.66_ | MGVLLTQRTL LSLVLALLFP SMASMEVQLV ESGGGLVQPG GSLRLSCAAS |
| | spCD28(trun)_ | GFNIKDTYMH WVRQAPGKGL EWVARIDPAN GNSKYADSVK GRFTISADTS |
| | CD28_CD40 | KNTAYLQMNS LRAEDTAVYY CAPFGYYVSD YAMAYWGQGT LVTVSSGGGG |
| | | SGGGGSGGGG SDIQLTQSPS SLSASVGDRV TITCRAGESV DIFGVGFLHW |
| | | YQQKPGKAPK LLIYRASNLE SGVPSRFSGS GSRTDFTLTI SSLQPEDFAT |
| | | YYCQQTNEDP YTFGQGTKVE IKAAAGSGGS GIIHVKGKHL CPSPLFPGPS |
| | | KPFWVLVVVG GVLACYSLLV TVAFIIFWVR SKRSRLLHSD YMNMTPRRPG |
| | | PTRKHYQPYA PPRDFAAYRS KKVAKKPTNK APHPKQEPQE INFPDDLPGS |
| | | NTAAPVQETL HGCQPVTQED GKESRISVQE RQ |
| 181 | CD8a_HuT84.66_ | MALPVTALLL PLALLLHAAR PEVQLVESGG GLVQPGGSLR LSCAASGFNI |
| | spCD28(trun)_ | KDTYMHWVRQ APGKGLEWVA RIDPANGNSK YADSVKGRFT ISADTSKNTA |
| | CD28_CD40 | YLQMNSLRAE DTAVYYCAPF GYYVSDYAMA YWGQGTLVTV SSGGGGSGGG |
| | | GSGGGGSDIQ LTQSPSSLSA SVGDRVTITC RAGESVDIFG VGFLHWYQQK |
| | | PGKAPKLLIY RASNLESGVP SRFSGSGSRT DFTLTISSLQ PEDFATYYCQ |
| | | QTNEDPYTFG QGTKVEIKAA AGSGGSGIIH VKGKHLCPSP LFPGPSKPFW |
| | | VLVVVGGVLA CYSLLVTVAF IIFWVRSKRS RLLHSDYMNM TPRRPGPTRK |
| | | HYQPYAPPRD FAAYRSKKVA KKPTNKAPHP KQEPQEINFP DDLPGSNTAA |
| | | PVQETLHGCQ PVTQEDGKES RISVQERQ |
| 182 | CD2_HuT84.66_ | MSFPCKFVAS FLLIFNVSSK GAVSEVQLVE SGGGLVQPGG SLRLSCAASG |
| | spCD28(trun)_ | FNIKDTYMHW VRQAPGKGLE WVARIDPANG NSKYADSVKG RFTISADTSK |
| | CD28_CD40 | NTAYLQMNSL RAEDTAVYYC APFGYYVSDY AMAYWGQGTL VTVSSGGGGS |
| | | GGGGSGGGGS DIQLTQSPSS LSASVGDRVT ITCRAGESVD IFGVGFLHWY |
| | | QQKPGKAPKL LIYRASNLES GVPSRFSGSG SRTDFTLTIS SLQPEDFATY |
| | | YCQQTNEDPY TFGQGTKVEI KAAAGSGGSG IIHVKGKHLC PSPLFPGPSK |
| | | PFWVLVVVGG VLACYSLLVT VAFIIFWVRS KRSRLLHSDY MNMTPRRPGP |
| | | TRKHYQPYAP PRDFAAYRSK KVAKKPTNKA PHPKQEPQEI NFPDDLPGSN |
| | | TAAPVQETLH GCQPVTQEDG KESRISVQER Q |
| 183 | IL2_HuT84.66_ | MYRMQLLSCI ALSLALVTNS EVQLVESGGG LVQPGGSLRL SCAASGFNIK |
| | spCD28(trun)_ | DTYMHWVRQA PGKGLEWVAR IDPANGNSKY ADSVKGRFTI SADTSKNTAY |
| | CD28_CD40 | LQMNSLRAED TAVYYCAPFG YYVSDYAMAY WGQGTLVTVS SGGGGSGGGG |
| | | SGGGGSDIQL TQSPSSLSAS VGDRVTITCR AGESVDIFGV GFLHWYQQKP |
| | | GKAPKLLIYR ASNLESGVPS RFSGSGSRTD FTLTISSLQP EDFATYYCQQ |
| | | TNEDPYTFGQ GTKVEIKAAA GSGGSGIIHV KGKHLCPSPL FPGPSKPFWV |
| | | LVVVGGVLAC YSLLVTVAFI IFWVRSKRSR LLHSDYMNMT PRRPGPTRKH |
| | | YQPYAPPRDF AAYRSKKVAK KPTNKAPHPK QEPQEINFPD DLPGSNTAAP |
| | | VQETLHGCQP VTQEDGKESR ISVQERQ |
| 184 | GM-CSF_ | MWLQSLLLLG TVACSISEVQ LVESGGGLVQ PGGSLRLSCA ASGFNIKDTY |
| | HuT84.66_ | MHWVRQAPGK GLEWVARIDP ANGNSKYADS VKGRFTISAD TSKNTAYLQM |
| | spCD28(trun)_ | NSLRAEDTAV YYCAPFGYYV SDYAMAYWGQ GTLVTVSSGG GGSGGGGSGG |
| | CD28_CD40 | GGSDIQLTQS PSSLSASVGD RVTITCRAGE SVDIFGVGFL HWYQQKPGKA |
| | | PKLLIYRASN LESGVPSRFS GSGSRTDFTL TISSLQPEDF ATYYCQQTNE |
| | | DPYTFGQGTK VEIKAAAGSG GSGIIHVKGK HLCPSPLFPG PSKPFWVLVV |
| | | VGGVLACYSL LVTVAFIIFW VRSKRSRLLH SDYMNMTPRR PGPTRKHYQP |
| | | YAPPRDFAAY RSKKVAKKPT NKAPHPKQEP QEINFPDDLP GSNTAAPVQE |
| | | TLHGCQPVTQ EDGKESRISV QERQ |
| 185 | hIgGk- | MEAPAQLLFL LLLWLPDTTR EVQLVESGGG LVQPGGSLRL SCAASGFNIK |
| | VIII_HuT84.66_ | DTYMHWVRQA PGKGLEWVAR IDPANGNSKY ADSVKGRFTI SADTSKNTAY |
| | spCD28(trun)_ | LQMNSLRAED TAVYYCAPFG YYVSDYAMAY WGQGTLVTVS SGGGGSGGGG |
| | CD28_CD40 | SGGGGSDIQL TQSPSSLSAS VGDRVTITCR AGESVDIFGV GFLHWYQQKP |
| | | GKAPKLLIYR ASNLESGVPS RFSGSGSRTD FTLTISSLQP EDFATYYCQQ |
| | | TNEDPYTFGQ GTKVEIKAAA GSGGSGIIHV KGKHLCPSPL FPGPSKPFWV |
| | | LVVVGGVLAC YSLLVTVAFI IFWVRSKRSR LLHSDYMNMT PRRPGPTRKH |
| | | YQPYAPPRDF AAYRSKKVAK KPTNKAPHPK QEPQEINFPD DLPGSNTAAP |
| | | VQETLHGCQP VTQEDGKESR ISVQERQ |
| 186 | SS1 | QVQLQQSGPE LEKPGASVKL SCKASGYSFT GYTMNWVKQS HGKSLEWIGL |
| | | ITPYNGASSY NQKFRGKATL TVDKSSSTAY MDLLSLTSED SAVYFCARGG |
| | | YDGRGFDYWG QGTTVTVSSG GGGSGGGGSG GGGSDIELTQ SPAIMSASPG |
| | | EKVTMTCSAS SSVSYMHWYQ QKSGTSPKRW IYDTSKLASG VPGRFSGSGS |
| | | GNSYSLTISS VEAEDDATYY CQQWSKHPLT FGAGTKLEIK |
| 187 | M5 | QVQLVQSGAE VEKPGASVKV SCKASGYTFT DYYMHWVRQA PGQGLEWMGW |
| | (humanized SS1) | INPNSGGTNY AQKFQGRVTM TRDTSISTAY MELSRLRSDD TAVYYCASGW |
| | | DFDYWGQGTL VTVSSGGGGS GGGGSGGGGS DIVMTQSPSS LSASVGDRVT |
| | | ITCRASQSIR YYLSWYQQKP GKAPKLLIYT ASILQNGVPS RFSGSGSGTD |
| | | FTLTISSLQP EDFATYYCLQ TYTTPDFGPG TKVEIK |
| 188 | HN1 | QVQLVQSGAE VKRPGASVQV SCRASGYSIN TYYMQWVRQA PGAGLEWMGV |
| | | INPSGVTSYA QKFQGRVTLT NDTSTNTVYM QLNSLTSADT AVYYCARWAL |
| | | WGDFGMDVWG KGTLVTVSSG GGGSGGGGSG GGGSDIQMTQ SPSTLSASIG |
| | | DRVTITCRAS EGIYHWLAWY QQKPGKAPKL LIYKASSLAS GAPSRFSGSG |
| | | SGTDFTLTIS SLQPDDFATY YCQQYSNYPL TFGGGTKLEI K |
| 189 | M912 | QVQLQESGPG LVKPSETLSL TCTVSGGSVS SGSYYWSWIR QPPGKGLEWI |
| | | GYIYYSGSTN YNPSLKSRVT ISVDTSKNQF SLKLSSVTAA DTAVYYCARE |
| | | GKNGAFDIWG QGTMVTVSSG GGGSGGGGSG GGGSDIQMTQ SPSSLSASVG |
| | | DRVTITCRAS QSISSYLNWY QQKPGKAPKL LIYAASSLQS GVPSGFSGSG |
| | | SGTDFTLTIS SLQPEDFATY YCQQSYSTPL TFGGGTKVEI K |
| 190 | HuYP218 | EVQLVESGGG LVQPGGSLRL SCAASGFDLG FYFYACWVRQ APGKGLEWVS |
| | | GIYTAGSGST YYASWAKGRF TISRDNSKNT LYLQMNSLRA EDTAVYYCAR |
| | | STANTRSTYY LNLWGQGTLV TVSSGGGGSG GGGSGGGGSD IQMTQSPSSL |
| | | SASVGDRVTI TCQASQRISS YLSWYQQKPG KVPKLLIYGA STLASGVPSR |
| | | FSGSGSGTDF TLTISSLQPE DVATYYCQSY AYFDSNNWHA FGGGTKVEI |
| 191 | P4 | QVQLQQSGPG LVTPSQTLSL TCAISGDSVS SNSATWNWIR QSPSRGLEWL |
| | | GRTYYRSKWY NDYAVSVKSR MSINPDTSKN QFSLQLNSVT PEDTAVYYCA |
| | | RGMMTYYYGM DVWGQGTTVT VSSGGGGSGG GGSGGGGSQP VLTQSSSLSA |
| | | SPGASASLTC TLRSGINVGP YRIYWYQQKP GSPPQYLLNY KSDSDKQQGS |
| | | GVPSRFSGSK DASANAGVLL ISGLRSEDEA DYYCMIWHSS AAVFGGGTQL |
| | | TVLS |
| 192 | OSM_SS1_ | MGVLLTQRTL LSLVLALLFP SMASMQVQLQ QSGPELEKPG ASVKLSCKAS |
| | spCD28_ | GYSFTGYTMN WVKQSHGKSL EWIGLITPYN GASSYNQKFR GKATLTVDKS |
| | CD28_CD40 | SSTAYMDLLS LTSEDSAVYF CARGGYDGRG FDYWGQGTTV TVSSGGGGSG |
| | CTP224 | GGGSGGGGSD IELTQSPAIM SASPGEKVTM TCSASSSVSY MHWYQQKSGT |
| | | SPKRWIYDTS KLASGVPGRF SGSGSGNSYS LTISSVEAED DATYYCQQWS |
| | | KHPLTFGAGT KLEIKAAAGS GGSGILVKQS PMLVAYDNAV NLSCKYSYNL |
| | | FSREFRASLH KGLDSAVEVC VVYGNYSQQL QVYSKTGFNC DGKLGNESVT |
| | | FYLQNLYVNQ TDIYFCKIEV MYPPPYLDNE KSNGTIIHVK GKHLCPSPLF |
| | | PGPSKPFWVL VVVGGVLACY SLLVTVAFII FWVRSKRSRL LHSDYMNMTP |
| | | RRPGPTRKHY QPYAPPRDFA AYRSKKVAKK PTNKAPHPKQ EPQEINFPDD |
| | | LPGSNTAAPV QETLHGCQPV TQEDGKESRI SVQERQ |
| 193 | CD8a_SS1_ | MALPVTALLL PLALLLHAAR PQVQLQQSGP ELEKPGASVK LSCKASGYSF |
| | spCD28_ | TGYTMNWVKQ SHGKSLEWIG LITPYNGASS YNQKFRGKAT LTVDKSSSTA |
| | CD28_CD40 | YMDLLSLTSE DSAVYFCARG GYDGRGFDYW GQGTTVTVSS GGGGSGGGGS |
| | | GGGGSDIELT QSPAIMSASP GEKVTMTCSA SSSVSYMHWY QQKSGTSPKR |
| | | WIYDTSKLAS GVPGRFSGSG SGNSYSLTIS SVEAEDDATY YCQQWSKHPL |
| | | TFGAGTKLEI KAAAGSGGSG ILVKQSPMLV AYDNAVNLSC KYSYNLFSRE |
| | | FRASLHKGLD SAVEVCVVYG NYSQQLQVYS KTGFNCDGKL GNESVTFYLQ |
| | | NLYVNQTDIY FCKIEVMYPP PYLDNEKSNG TIIHVKGKHL CPSPLFPGPS |
| | | KPFWVLVVVG GVLACYSLLV TVAFIIFWVR SKRSRLLHSD YMNMTPRRPG |
| | | PTRKHYQPYA PPRDFAAYRS KKVAKKPTNK APHPKQEPQE INFPDDLPGS |
| | | NTAAPVQETL HGCQPVTQED GKESRISVQE RQ |
| 194 | CD2_SS1_ | MSFPCKFVAS FLLIFNVSSK GAVSQVQLQQ SGPELEKPGA SVKLSCKASG |
| | spCD28_ | YSFTGYTMNW VKQSHGKSLE WIGLITPYNG ASSYNQKFRG KATLTVDKSS |
| | CD28_CD40 | STAYMDLLSL TSEDSAVYFC ARGGYDGRGF DYWGQGTTVT VSSGGGGSGG |
| | | GGSGGGGSDI ELTQSPAIMS ASPGEKVTMT CSASSSVSYM HWYQQKSGTS |
| | | PKRWIYDTSK LASGVPGRFS GSGSGNSYSL TISSVEAEDD ATYYCQQWSK |
| | | HPLTFGAGTK LEIKAAAGSG GSGILVKQSP MLVAYDNAVN LSCKYSYNLF |
| | | SREFRASLHK GLDSAVEVCV VYGNYSQQLQ VYSKTGFNCD GKLGNESVTF |
| | | YLQNLYVNQT DIYFCKIEVM YPPPYLDNEK SNGTIIHVKG KHLCPSPLFP |
| | | GPSKPFWVLV VVGGVLACYS LLVTVAFIIF WVRSKRSRLL HSDYMNMTPR |
| | | RPGPTRKHYQ PYAPPRDFAA YRSKKVAKKP TNKAPHPKQE PQEINFPDDL |
| | | PGSNTAAPVQ ETLHGCQPVT QEDGKESRIS VQERQ |
| 195 | IL2_SS1_ | MYRMQLLSCI ALSLALVTNS QVQLQQSGPE LEKPGASVKL SCKASGYSFT |
| | spCD28_ | GYTMNWVKQS HGKSLEWIGL ITPYNGASSY NQKFRGKATL TVDKSSSTAY |
| | CD28_CD40 | MDLLSLTSED SAVYFCARGG YDGRGFDYWG QGTTVTVSSG GGGSGGGGSG |
| | | GGGSDIELTQ SPAIMSASPG EKVTMTCSAS SSVSYMHWYQ QKSGTSPKRW |
| | | IYDTSKLASG VPGRFSGSGS GNSYSLTISS VEAEDDATYY CQQWSKHPLT |
| | | FGAGTKLEIK AAAGSGGSGI LVKQSPMLVA YDNAVNLSCK YSYNLFSREF |
| | | RASLHKGLDS AVEVCVVYGN YSQQLQVYSK TGFNCDGKLG NESVTFYLQN |
| | | LYVNQTDIYF CKIEVMYPPP YLDNEKSNGT IIHVKGKHLC PSPLFPGPSK |
| | | PFWVLVVVGG VLACYSLLVT VAFIIFWVRS KRSRLLHSDY MNMTPRRPGP |
| | | TRKHYQPYAP PRDFAAYRSK KVAKKPTNKA PHPKQEPQEI NFPDDLPGSN |
| | | TAAPVQETLH GCQPVTQEDG KESRISVQER Q |
| 196 | GM-CSF_SS1_ | MWLQSLLLLG TVACSISQVQ LQQSGPELEK PGASVKLSCK ASGYSFTGYT |
| | spCD28_ | MNWVKQSHGK SLEWIGLITP YNGASSYNQK FRGKATLTVD KSSSTAYMDL |
| | CD28_CD40 | LSLTSEDSAV YFCARGGYDG RGFDYWGQGT TVTVSSGGGG SGGGGSGGGG |
| | | SDIELTQSPA IMSASPGEKV TMTCSASSSV SYMHWYQQKS GTSPKRWIYD |
| | | TSKLASGVPG RFSGSGSGNS YSLTISSVEA EDDATYYCQQ WSKHPLTFGA |
| | | GTKLEIKAAA GSGGSGILVK QSPMLVAYDN AVNLSCKYSY NLFSREFRAS |
| | | LHKGLDSAVE VCVVYGNYSQ QLQVYSKTGF NCDGKLGNES VTFYLQNLYV |
| | | NQTDIYFCKI EVMYPPPYLD NEKSNGTIIH VKGKHLCPSP LFPGPSKPFW |
| | | VLVVVGGVLA CYSLLVTVAF IIFWVRSKRS RLLHSDYMNM TPRRPGPTRK |
| | | HYQPYAPPRD FAAYRSKKVA KKPTNKAPHP KQEPQEINFP DDLPGSNTAA |
| | | PVQETLHGCQ PVTQEDGKES RISVQERQ |
| 197 | hIgGk-VIII_ | MEAPAQLLFL LLLWLPDTTR QVQLQQSGPE LEKPGASVKL SCKASGYSFT |
| | SS1_spCD28_ | GYTMNWVKQS HGKSLEWIGL ITPYNGASSY NQKFRGKATL TVDKSSSTAY |
| | CD28_CD40 | MDLLSLTSED SAVYFCARGG YDGRGFDYWG QGTTVTVSSG GGGSGGGGSG |
| | | GGGSDIELTQ SPAIMSASPG EKVTMTCSAS SSVSYMHWYQ QKSGTSPKRW |
| | | IYDTSKLASG VPGRFSGSGS GNSYSLTISS VEAEDDATYY CQQWSKHPLT |
| | | FGAGTKLEIK AAAGSGGSGI LVKQSPMLVA YDNAVNLSCK YSYNLFSREF |
| | | RASLHKGLDS AVEVCVVYGN YSQQLQVYSK TGFNCDGKLG NESVTFYLQN |
| | | LYVNQTDIYF CKIEVMYPPP YLDNEKSNGT IIHVKGKHLC PSPLFPGPSK |
| | | PFWVLVVVGG VLACYSLLVT VAFIIFWVRS KRSRLLHSDY MNMTPRRPGP |
| | | TRKHYQPYAP PRDFAAYRSK KVAKKPTNKA PHPKQEPQEI NFPDDLPGSN |
| | | TAAPVQETLH GCQPVTQEDG KESRISVQER Q |
| 198 | OSM_SS1_spCD8_ | MGVLLTQRTL LSLVLALLFP SMASMQVOLQ QSGPELEKPG ASVKLSCKAS |
| | CD28_CD40 | GYSFTGYTMN WVKQSHGKSL EWIGLITPYN GASSYNQKFR GKATLTVDKS |
| | CTP236 | SSTAYMDLLS LTSEDSAVYF CARGGYDGRG FDYWGQGTTV TVSSGGGGSG |
| | | GGGSGGGGSD IELTQSPAIM SASPGEKVTM TCSASSSVSY MHWYQQKSGT |
| | | SPKRWIYDTS KLASGVPGRF SGSGSGNSYS LTISSVEAED DATYYCQQWS |
| | | KHPLTFGAGT KLEIKAAAGS GGSGFVPVFL PAKPTTTPAP RPPTPAPTIA |
| | | SQPLSLRPEA CRPAAGGAVH TRGLDFACDI YIWAPLAGTC GVLLLSLVIT |
| | | LYCNHRNRSK RSRLLHSDYM NMTPRRPGPT RKHYQPYAPP RDFAAYRSKK |
| | | VAKKPTNKAP HPKQEPQEIN FPDDLPGSNT AAPVQETLHG CQPVTQEDGK |
| | | ESRISVQERQ |
| 199 | CD8a_SS1_ | MALPVTALLL PLALLLHAAR PQVQLQQSGP ELEKPGASVK LSCKASGYSF |
| | spCD8_ | TGYTMNWVKQ SHGKSLEWIG LITPYNGASS YNQKFRGKAT LTVDKSSSTA |
| | CD28_CD40 | YMDLLSLTSE DSAVYFCARG GYDGRGFDYW GQGTTVTVSS GGGGSGGGGS |
| | | GGGGSDIELT QSPAIMSASP GEKVTMTCSA SSSVSYMHWY QQKSGTSPKR |
| | | WIYDTSKLAS GVPGRFSGSG SGNSYSLTIS SVEAEDDATY YCQQWSKHPL |
| | | TFGAGTKLEI KAAAGSGGSG FVPVFLPAKP TTTPAPRPPT PAPTIASQPL |
| | | SLRPEACRPA AGGAVHTRGL DFACDIYIWA PLAGTCGVLL LSLVITLYCN |
| | | HRNRSKRSRL LHSDYMNMTP RRPGPTRKHY QPYAPPRDFA AYRSKKVAKK |
| | | PTNKAPHPKQ EPQEINFPDD LPGSNTAAPV QETLHGCQPV TQEDGKESRI |
| | | SVQERQ |
| 200 | CD2_SS1_spCD8_ | MSFPCKFVAS FLLIFNVSSK GAVSQVQLQQ SGPELEKPGA SVKLSCKASG |
| | CD28_CD40 | YSFTGYTMNW VKQSHGKSLE WIGLITPYNG ASSYNQKFRG KATLTVDKSS |
| | | STAYMDLLSL TSEDSAVYFC ARGGYDGRGF DYWGQGTTVT VSSGGGGSGG |
| | | GGSGGGGSDI ELTQSPAIMS ASPGEKVTMT CSASSSVSYM HWYQQKSGTS |
| | | PKRWIYDTSK LASGVPGRFS GSGSGNSYSL TISSVEAEDD ATYYCQQWSK |
| | | HPLTFGAGTK LEIKAAAGSG GSGFVPVFLP AKPTTTPAPR PPTPAPTIAS |
| | | QPLSLRPEAC RPAAGGAVHT RGLDFACDIY IWAPLAGTCG VLLLSLVITL |
| | | YCNHRNRSKR SRLLHSDYMN MTPRRPGPTR KHYQPYAPPR DFAAYRSKKV |
| | | AKKPTNKAPH PKQEPQEINF PDDLPGSNTA APVQETLHGC QPVTQEDGKE |
| | | SRISVQERQ |
| 201 | IL2_SS1_spCD8_ | MYRMQLLSCI ALSLALVTNS QVQLQQSGPE LEKPGASVKL SCKASGYSFT |
| | CD28_CD40 | GYTMNWVKQS HGKSLEWIGL ITPYNGASSY NQKFRGKATL TVDKSSSTAY |
| | | MDLLSLTSED SAVYFCARGG YDGRGFDYWG QGTTVTVSSG GGGSGGGGSG |
| | | GGGSDIELTQ SPAIMSASPG EKVTMTCSAS SSVSYMHWYQ QKSGTSPKRW |
| | | IYDTSKLASG VPGRFSGSGS GNSYSLTISS VEAEDDATYY CQQWSKHPLT |
| | | FGAGTKLEIK AAAGSGGSGF VPVFLPAKPT TTPAPRPPTP APTIASQPLS |
| | | LRPEACRPAA GGAVHTRGLD FACDIYIWAP LAGTCGVLLL SLVITLYCNH |
| | | RNRSKRSRLL HSDYMNMTPR RPGPTRKHYQ PYAPPRDFAA YRSKKVAKKP |
| | | TNKAPHPKQE PQEINFPDDL PGSNTAAPVQ ETLHGCQPVT QEDGKESRIS |
| | | VQERQ |
| 202 | GM-CSF_SS1_ | MWLQSLLLLG TVACSISQVQ LQQSGPELEK PGASVKLSCK ASGYSFTGYT |
| | spCD8_ | MNWVKQSHGK SLEWIGLITP YNGASSYNQK FRGKATLTVD KSSSTAYMDL |
| | CD28_CD40 | LSLTSEDSAV YFCARGGYDG RGFDYWGQGT TVTVSSGGGG SGGGGSGGGG |
| | | SDIELTQSPA IMSASPGEKV TMTCSASSSV SYMHWYQQKS GTSPKRWIYD |
| | | TSKLASGVPG RFSGSGSGNS YSLTISSVEA EDDATYYCQQ WSKHPLTFGA |
| | | GTKLEIKAAA GSGGSGFVPV FLPAKPTTTP APRPPTPAPT IASQPLSLRP |
| | | EACRPAAGGA VHTRGLDFAC DIYIWAPLAG TCGVLLLSLV ITLYCNHRNR |
| | | SKRSRLLHSD YMNMTPRRPG PTRKHYQPYA PPRDFAAYRS KKVAKKPTNK |
| | | APHPKQEPQE INFPDDLPGS NTAAPVQETL HGCQPVTQED GKESRISVQE |
| | | RQ |
| 203 | hIgGk-VIII_ | MEAPAQLLFL LLLWLPDTTR QVQLQQSGPE LEKPGASVKL SCKASGYSFT |
| | SS1_spCD8_ | GYTMNWVKQS HGKSLEWIGL ITPYNGASSY NQKFRGKATL TVDKSSSTAY |
| | CD28_CD40 | MDLLSLTSED SAVYFCARGG YDGRGFDYWG QGTTVTVSSG GGGSGGGGSG |
| | | GGGSDIELTQ SPAIMSASPG EKVTMTCSAS SSVSYMHWYQ QKSGTSPKRW |
| | | IYDTSKLASG VPGRFSGSGS GNSYSLTISS VEAEDDATYY CQQWSKHPLT |
| | | FGAGTKLEIK AAAGSGGSGF VPVFLPAKPT TTPAPRPPTP APTIASQPLS |
| | | LRPEACRPAA GGAVHTRGLD FACDIYIWAP LAGTCGVLLL SLVITLYCNH |
| | | RNRSKRSRLL HSDYMNMTPR RPGPTRKHYQ PYAPPRDFAA YRSKKVAKKP |
| | | TNKAPHPKQE PQEINFPDDL PGSNTAAPVQ ETLHGCQPVT QEDGKESRIS |
| | | VQERQ |
| 204 | OSM_SS1_ | MGVLLTQRTL LSLVLALLFP SMASMQVQLQ QSGPELEKPG ASVKLSCKAS |
| | CD28TM_ | GYSFTGYTMN WVKQSHGKSL EWIGLITPYN GASSYNQKFR GKATLTVDKS |
| | CD28_CD40 | SSTAYMDLLS LTSEDSAVYF CARGGYDGRG FDYWGQGTTV TVSSGGGGSG |
| | | GGGSGGGGSD IELTQSPAIM SASPGEKVTM TCSASSSVSY MHWYQQKSGT |
| | | SPKRWIYDTS KLASGVPGRF SGSGSGNSYS LTISSVEAED DATYYCQQWS |
| | | KHPLTFGAGT KLEIKAAAGS GGSGFWVLVV VGGVLACYSL LVTVAFIIFW |
| | | VRSKRSRLLH SDYMNMTPRR PGPTRKHYQP YAPPRDFAAY RSKKVAKKPT |
| | | NKAPHPKQEP QEINFPDDLP GSNTAAPVQE TLHGCQPVTQ EDGKESRISV |
| | | QERQ |
| 205 | CD8a_SS1_ | MALPVTALLL PLALLLHAAR PQVQLQQSGP ELEKPGASVK LSCKASGYSF |
| | CD28TM_ | TGYTMNWVKQ SHGKSLEWIG LITPYNGASS YNQKFRGKAT LTVDKSSSTA |
| | CD28_CD40 | YMDLLSLTSE DSAVYFCARG GYDGRGFDYW GQGTTVTVSS GGGGSGGGGS |
| | | GGGGSDIELT QSPAIMSASP GEKVTMTCSA SSSVSYMHWY QQKSGTSPKR |
| | | WIYDTSKLAS GVPGRFSGSG SGNSYSLTIS SVEAEDDATY YCQQWSKHPL |
| | | TFGAGTKLEI KAAAGSGGSG FWVLVVVGGV LACYSLLVTV AFIIFWVRSK |
| | | RSRLLHSDYM NMTPRRPGPT RKHYQPYAPP RDFAAYRSKK VAKKPTNKAP |
| | | HPKQEPQEIN FPDDLPGSNT AAPVQETLHG CQPVTQEDGK ESRISVQERQ |
| 206 | CD2_SS1_ | MSFPCKFVAS FLLIFNVSSK GAVSQVQLQQ SGPELEKPGA SVKLSCKASG |
| | CD28TM_ | YSFTGYTMNW VKQSHGKSLE WIGLITPYNG ASSYNQKFRG KATLTVDKSS |
| | CD28_CD40 | STAYMDLLSL TSEDSAVYFC ARGGYDGRGF DYWGQGTTVT VSSGGGGSGG |
| | | GGSGGGGSDI ELTQSPAIMS ASPGEKVTMT CSASSSVSYM HWYQQKSGTS |
| | | PKRWIYDTSK LASGVPGRFS GSGSGNSYSL TISSVEAEDD ATYYCQQWSK |
| | | HPLTFGAGTK LEIKAAAGSG GSGFWVLVVV GGVLACYSLL VTVAFIIFWV |
| | | RSKRSRLLHS DYMNMTPRRP GPTRKHYQPY APPRDFAAYR SKKVAKKPTN |
| | | KAPHPKQEPQ EINFPDDLPG SNTAAPVQET LHGCQPVTQE DGKESRISVQ |
| | | ERQ |
| 207 | IL2_SS1_ | MYRMQLLSCI ALSLALVTNS QVQLQQSGPE LEKPGASVKL SCKASGYSFT |
| | CD28TM_ | GYTMNWVKQS HGKSLEWIGL ITPYNGASSY NQKFRGKATL TVDKSSSTAY |
| | CD28_CD40 | MDLLSLTSED SAVYFCARGG YDGRGFDYWG QGTTVTVSSG GGGSGGGGSG |
| | | GGGSDIELTQ SPAIMSASPG EKVTMTCSAS SSVSYMHWYQ QKSGTSPKRW |
| | | IYDTSKLASG VPGRFSGSGS GNSYSLTISS VEAEDDATYY CQQWSKHPLT |
| | | FGAGTKLEIK AAAGSGGSGF WVLVVVGGVL ACYSLLVTVA FIIFWVRSKR |
| | | SRLLHSDYMN MTPRRPGPTR KHYQPYAPPR DFAAYRSKKV AKKPTNKAPH |
| | | PKQEPQEINF PDDLPGSNTA APVQETLHGC QPVTQEDGKE SRISVQERQ |
| 208 | GM-CSF_SS1_ | MWLQSLLLLG TVACSISQVQ LQQSGPELEK PGASVKLSCK ASGYSFTGYT |
| | CD28TM_ | MNWVKQSHGK SLEWIGLITP YNGASSYNQK FRGKATLTVD KSSSTAYMDL |
| | CD28_CD40 | LSLTSEDSAV YFCARGGYDG RGFDYWGQGT TVTVSSGGGG SGGGGSGGGG |
| | | SDIELTQSPA IMSASPGEKV TMTCSASSSV SYMHWYQQKS GTSPKRWIYD |
| | | TSKLASGVPG RFSGSGSGNS YSLTISSVEA EDDATYYCQQ WSKHPLTFGA |
| | | GTKLEIKAAA GSGGSGFWVL VVVGGVLACY SLLVTVAFII FWVRSKRSRL |
| | | LHSDYMNMTP RRPGPTRKHY QPYAPPRDFA AYRSKKVAKK PTNKAPHPKQ |
| | | EPQEINFPDD LPGSNTAAPV QETLHGCQPV TQEDGKESRI SVQERQ |
| 209 | hIgGk-VIII_ | MEAPAQLLFL LLLWLPDTTR QVQLQQSGPE LEKPGASVKL SCKASGYSFT |
| | SS1_CD28TM_ | GYTMNWVKQS HGKSLEWIGL ITPYNGASSY NQKFRGKATL TVDKSSSTAY |
| | CD28_CD40 | MDLLSLTSED SAVYFCARGG YDGRGFDYWG QGTTVTVSSG GGGSGGGGSG |
| | | GGGSDIELTQ SPAIMSASPG EKVTMTCSAS SSVSYMHWYQ QKSGTSPKRW |
| | | IYDTSKLASG VPGRFSGSGS GNSYSLTISS VEAEDDATYY CQQWSKHPLT |
| | | FGAGTKLEIK AAAGSGGSGF WVLVVVGGVL ACYSLLVTVA FIIFWVRSKR |
| | | SRLLHSDYMN MTPRRPGPTR KHYQPYAPPR DFAAYRSKKV AKKPTNKAPH |
| | | PKQEPQEINF PDDLPGSNTA APVQETLHGC QPVTQEDGKE SRISVQERQ |
| 210 | OSM_M5_ | MGVLLTQRTL LSLVLALLFP SMASMQVQLV QSGAEVEKPG ASVKVSCKAS |
| | spCD28_ | GYTFTDYYMH WVRQAPGQGL EWMGWINPNS GGTNYAQKFQ GRVTMTRDTS |
| | CD28_CD40 | ISTAYMELSR LRSDDTAVYY CASGWDFDYW GQGTLVTVSS GGGGSGGGGS |
| | CTP225 | GGGGSDIVMT QSPSSLSASV GDRVTITCRA SQSIRYYLSW YQQKPGKAPK |
| | | LLIYTASILQ NGVPSRFSGS GSGTDFTLTI SSLQPEDFAT YYCLQTYTTP |
| | | DFGPGTKVEI KAAAGSGGSG ILVKQSPMLV AYDNAVNLSC KYSYNLFSRE |
| | | FRASLHKGLD SAVEVCVVYG NYSQQLQVYS KTGFNCDGKL GNESVTFYLQ |
| | | NLYVNQTDIY FCKIEVMYPP PYLDNEKSNG TIIHVKGKHL CPSPLFPGPS |
| | | KPFWVLVVVG GVLACYSLLV TVAFIIFWVR SKRSRLLHSD YMNMTPRRPG |
| | | PTRKHYQPYA PPRDFAAYRS KKVAKKPTNK APHPKQEPQE INFPDDLPGS |
| | | NTAAPVQETL HGCQPVTQED GKESRISVQE RQ |
| 211 | CD8a_M5_ | MALPVTALLL PLALLLHAAR PQVQLVQSGA EVEKPGASVK VSCKASGYTF |
| | spCD28_ | TDYYMHWVRQ APGQGLEWMG WINPNSGGTN YAQKFQGRVT MTRDTSISTA |
| | CD28_CD40 | YMELSRLRSD DTAVYYCASG WDFDYWGQGT LVTVSSGGGG SGGGGSGGGG |
| | | SDIVMTQSPS SLSASVGDRV TITCRASQSI RYYLSWYQQK PGKAPKLLIY |
| | | TASILQNGVP SRFSGSGSGT DFTLTISSLQ PEDFATYYCL QTYTTPDFGP |
| | | GTKVEIKAAA GSGGSGILVK QSPMLVAYDN AVNLSCKYSY NLFSREFRAS |
| | | LHKGLDSAVE VCVVYGNYSQ QLQVYSKTGF NCDGKLGNES VTFYLQNLYV |
| | | NQTDIYFCKI EVMYPPPYLD NEKSNGTIIH VKGKHLCPSP LFPGPSKPFW |
| | | VLVVVGGVLA CYSLLVTVAF IIFWVRSKRS RLLHSDYMNM TPRRPGPTRK |
| | | HYQPYAPPRD FAAYRSKKVA KKPTNKAPHP KQEPQEINFP DDLPGSNTAA |
| | | PVQETLHGCQ PVTQEDGKES RISVQERQ |
| 212 | CD2_M5_ | MSFPCKFVAS FLLIFNVSSK GAVSQVQLVQ SGAEVEKPGA SVKVSCKASG |
| | spCD28_ | YTFTDYYMHW VRQAPGQGLE WMGWINPNSG GTNYAQKFQG RVTMTRDTSI |
| | CD28_CD40 | STAYMELSRL RSDDTAVYYC ASGWDFDYWG QGTLVTVSSG GGGSGGGGSG |
| | | GGGSDIVMTQ SPSSLSASVG DRVTITCRAS QSIRYYLSWY QQKPGKAPKL |
| | | LIYTASILQN GVPSRFSGSG SGTDFTLTIS SLQPEDFATY YCLQTYTTPD |
| | | FGPGTKVEIK AAAGSGGSGI LVKQSPMLVA YDNAVNLSCK YSYNLFSREF |
| | | RASLHKGLDS AVEVCVVYGN YSQQLQVYSK TGFNCDGKLG NESVTFYLQN |
| | | LYVNQTDIYF CKIEVMYPPP YLDNEKSNGT IIHVKGKHLC PSPLFPGPSK |
| | | PFWVLVVVGG VLACYSLLVT VAFIIFWVRS KRSRLLHSDY MNMTPRRPGP |
| | | TRKHYQPYAP PRDFAAYRSK KVAKKPTNKA PHPKQEPQEI NFPDDLPGSN |
| | | TAAPVQETLH GCQPVTQEDG KESRISVQER Q |
| 213 | IL2_M5_ | MYRMQLLSCI ALSLALVTNS QVQLVQSGAE VEKPGASVKV SCKASGYTFT |
| | spCD28_ | DYYMHWVRQA PGQGLEWMGW INPNSGGTNY AQKFQGRVTM TRDTSISTAY |
| | CD28_CD40 | MELSRLRSDD TAVYYCASGW DFDYWGQGTL VTVSSGGGGS GGGGSGGGGS |
| | | DIVMTQSPSS LSASVGDRVT ITCRASQSIR YYLSWYQQKP GKAPKLLIYT |
| | | ASILQNGVPS RFSGSGSGTD FTLTISSLQP EDFATYYCLQ TYTTPDFGPG |
| | | TKVEIKAAAG SGGSGILVKQ SPMLVAYDNA VNLSCKYSYN LFSREFRASL |
| | | HKGLDSAVEV CVVYGNYSQQ LQVYSKTGFN CDGKLGNESV TFYLQNLYVN |
| | | QTDIYFCKIE VMYPPPYLDN EKSNGTIIHV KGKHLCPSPL FPGPSKPFWV |
| | | LVVVGGVLAC YSLLVTVAFI IFWVRSKRSR LLHSDYMNMT PRRPGPTRKH |
| | | YQPYAPPRDF AAYRSKKVAK KPTNKAPHPK QEPQEINFPD DLPGSNTAAP |
| | | VQETLHGCQP VTQEDGKESR ISVQERQ |
| 214 | GM-CSF_M5_ | MWLQSLLLLG TVACSISQVQ LVQSGAEVEK PGASVKVSCK ASGYTFTDYY |
| | spCD28_ | MHWVRQAPGQ GLEWMGWINP NSGGTNYAQK FQGRVTMTRD TSISTAYMEL |
| | CD28_CD40 | SRLRSDDTAV YYCASGWDFD YWGQGTLVTV SSGGGGSGGG GSGGGGSDIV |
| | | MTQSPSSLSA SVGDRVTITC RASQSIRYYL SWYQQKPGKA PKLLIYTASI |
| | | LQNGVPSRFS GSGSGTDFTL TISSLQPEDF ATYYCLQTYT TPDFGPGTKV |
| | | EIKAAAGSGG SGILVKQSPM LVAYDNAVNL SCKYSYNLFS REFRASLHKG |
| | | LDSAVEVCVV YGNYSQQLQV YSKTGFNCDG KLGNESVTFY LQNLYVNQTD |
| | | IYFCKIEVMY PPPYLDNEKS NGTIIHVKGK HLCPSPLFPG PSKPFWVLVV |
| | | VGGVLACYSL LVTVAFIIFW VRSKRSRLLH SDYMNMTPRR PGPTRKHYQP |
| | | YAPPRDFAAY RSKKVAKKPT NKAPHPKQEP QEINFPDDLP GSNTAAPVQE |
| | | TLHGCQPVTQ EDGKESRISV QERQ |
| 215 | hIgGk-VIII_ | MEAPAQLLFL LLLWLPDTTR QVQLVQSGAE VEKPGASVKV SCKASGYTFT |
| | M5_ | DYYMHWVRQA PGQGLEWMGW INPNSGGTNY AQKFQGRVTM TRDTSISTAY |
| | spCD28_ | MELSRLRSDD TAVYYCASGW DFDYWGQGTL VTVSSGGGGS GGGGSGGGGS |
| | CD28_CD40 | DIVMTQSPSS LSASVGDRVT ITCRASQSIR YYLSWYQQKP GKAPKLLIYT |
| | | ASILQNGVPS RFSGSGSGTD FTLTISSLQP EDFATYYCLQ TYTTPDFGPG |
| | | TKVEIKAAAG SGGSGILVKQ SPMLVAYDNA VNLSCKYSYN LFSREFRASL |
| | | HKGLDSAVEV CVVYGNYSQQ LQVYSKTGFN CDGKLGNESV TFYLQNLYVN |
| | | QTDIYFCKIE VMYPPPYLDN EKSNGTIIHV KGKHLCPSPL FPGPSKPFWV |
| | | LVVVGGVLAC YSLLVTVAFI IFWVRSKRSR LLHSDYMNMT PRRPGPTRKH |
| | | YQPYAPPRDF AAYRSKKVAK KPTNKAPHPK QEPQEINFPD DLPGSNTAAP |
| | | VQETLHGCQP VTQEDGKESR ISVQERQ |
| 216 | OSM_M5_spCD8_ | MGVLLTQRTL LSLVLALLFP SMASMQVQLV QSGAEVEKPG ASVKVSCKAS |
| | CD28_CD40 | GYTFTDYYMH WVRQAPGQGL EWMGWINPNS GGTNYAQKFQ GRVTMTRDTS |
| | CTP237 | ISTAYMELSR LRSDDTAVYY CASGWDFDYW GQGTLVTVSS GGGGSGGGGS |
| | | GGGGSDIVMT QSPSSLSASV GDRVTITCRA SQSIRYYLSW YQQKPGKAPK |
| | | LLIYTASILQ NGVPSRFSGS GSGTDFTLTI SSLQPEDFAT YYCLQTYTTP |
| | | DFGPGTKVEI KAAAGSGGSG FVPVFLPAKP TTTPAPRPPT PAPTIASQPL |
| | | SLRPEACRPA AGGAVHTRGL DFACDIYIWA PLAGTCGVLL LSLVITLYCN |
| | | HRNRSKRSRL LHSDYMNMTP RRPGPTRKHY QPYAPPRDFA AYRSKKVAKK |
| | | PTNKAPHPKQ EPQEINFPDD LPGSNTAAPV QETLHGCQPV TQEDGKESRI |
| | | SVQERQ |
| 217 | CD8a_M5_spCD8_ | MALPVTALLL PLALLLHAAR PQVQLVQSGA EVEKPGASVK VSCKASGYTF |
| | CD28_CD40 | TDYYMHWVRQ APGQGLEWMG WINPNSGGTN YAQKFQGRVT MTRDTSISTA |
| | | YMELSRLRSD DTAVYYCASG WDFDYWGQGT LVTVSSGGGG SGGGGSGGGG |
| | | SDIVMTQSPS SLSASVGDRV TITCRASQSI RYYLSWYQQK PGKAPKLLIY |
| | | TASILQNGVP SRFSGSGSGT DFTLTISSLQ PEDFATYYCL QTYTTPDFGP |
| | | GTKVEIKAAA GSGGSGFVPV FLPAKPTTTP APRPPTPAPT IASQPLSLRP |
| | | EACRPAAGGA VHTRGLDFAC DIYIWAPLAG TCGVLLLSLV ITLYCNHRNR |
| | | SKRSRLLHSD YMNMTPRRPG PTRKHYQPYA PPRDFAAYRS KKVAKKPTNK |
| | | APHPKQEPQE INFPDDLPGS NTAAPVQETL HGCQPVTQED GKESRISVQE |
| | | RQ |
| 218 | CD2_M5_spCD8_ | MSFPCKFVAS FLLIFNVSSK GAVSQVQLVQ SGAEVEKPGA SVKVSCKASG |
| | CD28_CD40 | YTFTDYYMHW VRQAPGQGLE WMGWINPNSG GTNYAQKFQG RVTMTRDTSI |
| | | STAYMELSRL RSDDTAVYYC ASGWDFDYWG QGTLVTVSSG GGGSGGGGSG |
| | | GGGSDIVMTQ SPSSLSASVG DRVTITCRAS QSIRYYLSWY QQKPGKAPKL |
| | | LIYTASILON GVPSRFSGSG SGTDFTLTIS SLQPEDFATY YCLQTYTTPD |
| | | FGPGTKVEIK AAAGSGGSGF VPVFLPAKPT TTPAPRPPTP APTIASQPLS |
| | | LRPEACRPAA GGAVHTRGLD FACDIYIWAP LAGTCGVLLL SLVITLYCNH |
| | | RNRSKRSRLL HSDYMNMTPR RPGPTRKHYQ PYAPPRDFAA YRSKKVAKKP |
| | | TNKAPHPKQE PQEINFPDDL PGSNTAAPVQ ETLHGCQPVT QEDGKESRIS |
| | | VQERQ |
| 219 | IL2_M5_spCD8_ | MYRMQLLSCI ALSLALVTNS QVQLVQSGAE VEKPGASVKV SCKASGYTFT |
| | CD28_CD40 | DYYMHWVRQA PGQGLEWMGW INPNSGGTNY AQKFQGRVTM TRDTSISTAY |
| | | MELSRLRSDD TAVYYCASGW DFDYWGQGTL VTVSSGGGGS GGGGSGGGGS |
| | | DIVMTQSPSS LSASVGDRVT ITCRASQSIR YYLSWYQQKP GKAPKLLIYT |
| | | ASILQNGVPS RFSGSGSGTD FTLTISSLQP EDFATYYCLQ TYTTPDFGPG |
| | | TKVEIKAAAG SGGSGFVPVF LPAKPTTTPA PRPPTPAPTI ASQPLSLRPE |
| | | ACRPAAGGAV HTRGLDFACD IYIWAPLAGT CGVLLLSLVI TLYCNHRNRS |
| | | KRSRLLHSDY MNMTPRRPGP TRKHYQPYAP PRDFAAYRSK KVAKKPTNKA |
| | | PHPKQEPQEI NFPDDLPGSN TAAPVQETLH GCQPVTQEDG KESRISVQER |
| | | Q |
| 220 | GM-CSF_M5_ | MWLQSLLLLG TVACSISQVQ LVQSGAEVEK PGASVKVSCK ASGYTFTDYY |
| | spCD8_CD28_CD40 | MHWVRQAPGQ GLEWMGWINP NSGGTNYAQK FQGRVTMTRD TSISTAYMEL |
| | | SRLRSDDTAV YYCASGWDFD YWGQGTLVTV SSGGGGSGGG GSGGGGSDIV |
| | | MTQSPSSLSA SVGDRVTITC RASQSIRYYL SWYQQKPGKA PKLLIYTASI |
| | | LQNGVPSRFS GSGSGTDFTL TISSLQPEDF ATYYCLQTYT TPDFGPGTKV |
| | | EIKAAAGSGG SGFVPVFLPA KPTTTPAPRP PTPAPTIASQ PLSLRPEACR |
| | | PAAGGAVHTR GLDFACDIYI WAPLAGTCGV LLLSLVITLY CNHRNRSKRS |
| | | RLLHSDYMNM TPRRPGPTRK HYQPYAPPRD FAAYRSKKVA KKPTNKAPHP |
| | | KQEPQEINFP DDLPGSNTAA PVQETLHGCQ PVTQEDGKES RISVQERQ |
| 221 | hIgGk-VIII_ | MEAPAQLLFL LLLWLPDTTR QVQLVQSGAE VEKPGASVKV SCKASGYTFT |
| | M5_spCD8_ | DYYMHWVRQA PGQGLEWMGW INPNSGGTNY AQKFQGRVTM TRDTSISTAY |
| | CD28_CD40 | MELSRLRSDD TAVYYCASGW DFDYWGQGTL VTVSSGGGGS GGGGSGGGGS |
| | | DIVMTQSPSS LSASVGDRVT ITCRASQSIR YYLSWYQQKP GKAPKLLIYT |
| | | ASILQNGVPS RFSGSGSGTD FTLTISSLQP EDFATYYCLQ TYTTPDFGPG |
| | | TKVEIKAAAG SGGSGFVPVF LPAKPTTTPA PRPPTPAPTI ASQPLSLRPE |
| | | ACRPAAGGAV HTRGLDFACD IYIWAPLAGT CGVLLLSLVI TLYCNHRNRS |
| | | KRSRLLHSDY MNMTPRRPGP TRKHYQPYAP PRDFAAYRSK KVAKKPTNKA |
| | | PHPKQEPQEI NFPDDLPGSN TAAPVQETLH GCQPVTQEDG KESRISVQER |
| | | Q |
| 222 | OSM_M5_CD28TM_ | MGVLLTQRTL LSLVLALLFP SMASMQVQLV QSGAEVEKPG ASVKVSCKAS |
| | CD28_CD40 | GYTFTDYYMH WVRQAPGQGL EWMGWINPNS GGTNYAQKFQ GRVTMTRDTS |
| | | ISTAYMELSR LRSDDTAVYY CASGWDFDYW GQGTLVTVSS GGGGSGGGGS |
| | | GGGGSDIVMT QSPSSLSASV GDRVTITCRA SQSIRYYLSW YQQKPGKAPK |
| | | LLIYTASILQ NGVPSRFSGS GSGTDFTLTI SSLQPEDFAT YYCLQTYTTP |
| | | DFGPGTKVEI KAAAGSGGSG FWVLVVVGGV LACYSLLVTV AFIIFWVRSK |
| | | RSRLLHSDYM NMTPRRPGPT RKHYQPYAPP RDFAAYRSKK VAKKPTNKAP |
| | | HPKQEPQEIN FPDDLPGSNT AAPVQETLHG CQPVTQEDGK ESRISVQERQ |
| 223 | CD8a_M5_CD28TM_ | MALPVTALLL PLALLLHAAR PQVQLVQSGA EVEKPGASVK VSCKASGYTF |
| | CD28_CD40 | TDYYMHWVRQ APGQGLEWMG WINPNSGGTN YAQKFQGRVT MTRDTSISTA |
| | | YMELSRLRSD DTAVYYCASG WDFDYWGQGT LVTVSSGGGG SGGGGSGGGG |
| | | SDIVMTQSPS SLSASVGDRV TITCRASQSI RYYLSWYQQK PGKAPKLLIY |
| | | TASILQNGVP SRFSGSGSGT DFTLTISSLQ PEDFATYYCL QTYTTPDFGP |
| | | GTKVEIKAAA GSGGSGFWVL VVVGGVLACY SLLVTVAFII FWVRSKRSRL |
| | | LHSDYMNMTP RRPGPTRKHY QPYAPPRDFA AYRSKKVAKK PTNKAPHPKQ |
| | | EPQEINFPDD LPGSNTAAPV QETLHGCQPV TQEDGKESRI SVQERQ |
| 224 | CD2_M5_CD28TM_ | MSFPCKFVAS FLLIFNVSSK GAVSQVQLVQ SGAEVEKPGA SVKVSCKASG |
| | CD28_CD40 | YTFTDYYMHW VRQAPGQGLE WMGWINPNSG GTNYAQKFQG RVTMTRDTSI |
| | | STAYMELSRL RSDDTAVYYC ASGWDFDYWG QGTLVTVSSG GGGSGGGGSG |
| | | GGGSDIVMTQ SPSSLSASVG DRVTITCRAS QSIRYYLSWY QQKPGKAPKL |
| | | LIYTASILON GVPSRFSGSG SGTDFTLTIS SLQPEDFATY YCLQTYTTPD |
| | | FGPGTKVEIK AAAGSGGSGF WVLVVVGGVL ACYSLLVTVA FIIFWVRSKR |
| | | SRLLHSDYMN MTPRRPGPTR KHYQPYAPPR DFAAYRSKKV AKKPTNKAPH |
| | | PKQEPQEINF PDDLPGSNTA APVQETLHGC QPVTQEDGKE SRISVQERQ |
| 225 | IL2_M5_CD28TM_ | MYRMQLLSCI ALSLALVTNS QVQLVQSGAE VEKPGASVKV SCKASGYTFT |
| | CD28_CD40 | DYYMHWVRQA PGQGLEWMGW INPNSGGTNY AQKFQGRVTM TRDTSISTAY |
| | | MELSRLRSDD TAVYYCASGW DFDYWGQGTL VTVSSGGGGS GGGGSGGGGS |
| | | DIVMTQSPSS LSASVGDRVT ITCRASQSIR YYLSWYQQKP GKAPKLLIYT |
| | | ASILQNGVPS RFSGSGSGTD FTLTISSLQP EDFATYYCLQ TYTTPDFGPG |
| | | TKVEIKAAAG SGGSGFWVLV VVGGVLACYS LLVTVAFIIF WVRSKRSRLL |
| | | HSDYMNMTPR RPGPTRKHYQ PYAPPRDFAA YRSKKVAKKP TNKAPHPKQE |
| | | PQEINFPDDL PGSNTAAPVQ ETLHGCQPVT QEDGKESRIS VQERQ |
| 226 | GM-CSF_M5_ | MWLQSLLLLG TVACSISQVQ LVQSGAEVEK PGASVKVSCK ASGYTFTDYY |
| | CD28TM_ | MHWVRQAPGQ GLEWMGWINP NSGGTNYAQK FQGRVTMTRD TSISTAYMEL |
| | CD28_CD40 | SRLRSDDTAV YYCASGWDFD YWGQGTLVTV SSGGGGSGGG GSGGGGSDIV |
| | | MTQSPSSLSA SVGDRVTITC RASQSIRYYL SWYQQKPGKA PKLLIYTASI |
| | | LQNGVPSRFS GSGSGTDFTL TISSLQPEDF ATYYCLQTYT TPDFGPGTKV |
| | | EIKAAAGSGG SGFWVLVVVG GVLACYSLLV TVAFIIFWVR SKRSRLLHSD |
| | | YMNMTPRRPG PTRKHYQPYA PPRDFAAYRS KKVAKKPTNK APHPKQEPQE |
| | | INFPDDLPGS NTAAPVQETL HGCQPVTQED GKESRISVQE RQ |
| 227 | hIgGk-VIII_ | MEAPAQLLFL LLLWLPDTTR QVQLVQSGAE VEKPGASVKV SCKASGYTFT |
| | M5_CD28TM_ | DYYMHWVRQA PGQGLEWMGW INPNSGGTNY AQKFQGRVTM TRDTSISTAY |
| | CD28_CD40 | MELSRLRSDD TAVYYCASGW DFDYWGQGTL VTVSSGGGGS GGGGSGGGGS |
| | | DIVMTQSPSS LSASVGDRVT ITCRASQSIR YYLSWYQQKP GKAPKLLIYT |
| | | ASILQNGVPS RFSGSGSGTD FTLTISSLQP EDFATYYCLQ TYTTPDFGPG |
| | | TKVEIKAAAG SGGSGFWVLV VVGGVLACYS LLVTVAFIIF WVRSKRSRLL |
| | | HSDYMNMTPR RPGPTRKHYQ PYAPPRDFAA YRSKKVAKKP TNKAPHPKQE |
| | | PQEINFPDDL PGSNTAAPVQ ETLHGCQPVT QEDGKESRIS VQERQ |
| 228 | OSM_HN1_ | MGVLLTQRTL LSLVLALLFP SMASMQVQLV QSGAEVKRPG ASVQVSCRAS |
| | spCD28_ | GYSINTYYMQ WVRQAPGAGL EWMGVINPSG VTSYAQKFQG RVTLTNDTST |
| | CD28_CD40 | NTVYMQLNSL TSADTAVYYC ARWALWGDFG MDVWGKGTLV TVSSGGGGSG |
| | CTP226 | GGGSGGGGSD IQMTQSPSTL SASIGDRVTI TGRASEGIYH WLAWYQQKPG |
| | | KAPKLLIYKA SSLASGAPSR FSGSGSGTDF TLTISSLQPD DFATYYCQQY |
| | | SNYPLTFGGG TKLEIKAAAG SGGSGILVKQ SPMLVAYDNA VNLSCKYSYN |
| | | LFSREFRASL HKGLDSAVEV CVVYGNYSQQ LQVYSKTGFN CDGKLGNESV |
| | | TFYLQNLYVN QTDIYFCKIE VMYPPPYLDN EKSNGTIIHV KGKHLCPSPL |
| | | FPGPSKPFWV LVVVGGVLAC YSLLVTVAFI IFWVRSKRSR LLHSDYMNMT |
| | | PRRPGPTRKH YQPYAPPRDF AAYRSKKVAK KPTNKAPHPK QEPQEINFPD |
| | | DLPGSNTAAP VQETLHGCQP VTQEDGKESR ISVQERQ |
| 229 | CD8a_HN1_ | MALPVTALLL PLALLLHAAR PQVQLVQSGA EVKRPGASVQ VSCRASGYSI |
| | spCD28_ | NTYYMQWVRQ APGAGLEWMG VINPSGVTSY AQKFQGRVTL TNDTSTNTVY |
| | CD28_CD40 | MQLNSLTSAD TAVYYCARWA LWGDFGMDVW GKGTLVTVSS GGGGSGGGGS |
| | | GGGGSDIQMT QSPSTLSASI GDRVTITCRA SEGIYHWLAW YQQKPGKAPK |
| | | LLIYKASSLA SGAPSRFSGS GSGTDFTLTI SSLQPDDFAT YYCQQYSNYP |
| | | LTFGGGTKLE IKAAAGSGGS GILVKQSPML VAYDNAVNLS CKYSYNLFSR |
| | | EFRASLHKGL DSAVEVCVVY GNYSQQLQVY SKTGFNCDGK LGNESVTFYL |
| | | QNLYVNQTDI YFCKIEVMYP PPYLDNEKSN GTIIHVKGKH LCPSPLFPGP |
| | | SKPFWVLVVV GGVLACYSLL VTVAFIIFWV RSKRSRLLHS DYMNMTPRRP |
| | | GPTRKHYQPY APPRDFAAYR SKKVAKKPTN KAPHPKQEPQ EINFPDDLPG |
| | | SNTAAPVQET LHGCQPVTQE DGKESRISVQ ERQ |
| 230 | CD2_HN1_ | MSFPCKFVAS FLLIFNVSSK GAVSQVQLVQ SGAEVKRPGA SVQVSCRASG |
| | spCD28_ | YSINTYYMQW VRQAPGAGLE WMGVINPSGV TSYAQKFQGR VTLTNDTSTN |
| | CD28_CD40 | TVYMQLNSLT SADTAVYYGA RWALWGDFGM DVWGKGTLVT VSSGGGGSGG |
| | | GGSGGGGSDI QMTQSPSTLS ASIGDRVTIT CRASEGIYHW LAWYQQKPGK |
| | | APKLLIYKAS SLASGAPSRF SGSGSGTDFT LTISSLQPDD FATYYCQQYS |
| | | NYPLTFGGGT KLEIKAAAGS GGSGILVKQS PMLVAYDNAV NLSCKYSYNL |
| | | FSREFRASLH KGLDSAVEVC VVYGNYSQQL QVYSKTGFNC DGKLGNESVT |
| | | FYLQNLYVNQ TDIYFCKIEV MYPPPYLDNE KSNGTIIHVK GKHLCPSPLF |
| | | PGPSKPFWVL VVVGGVLACY SLLVTVAFII FWVRSKRSRL LHSDYMNMTP |
| | | RRPGPTRKHY QPYAPPRDFA AYRSKKVAKK PTNKAPHPKQ EPQEINFPDD |
| | | LPGSNTAAPV QETLHGCQPV TQEDGKESRI SVQERQ |
| 231 | IL2_HN1_ | MYRMQLLSCI ALSLALVTNS QVQLVQSGAE VKRPGASVQV SCRASGYSIN |
| | spCD28_ | TYYMQWVRQA PGAGLEWMGV INPSGVTSYA QKFQGRVTLT NDTSTNTVYM |
| | CD28_CD40 | QLNSLTSADT AVYYCARWAL WGDFGMDVWG KGTLVTVSSG GGGSGGGGSG |
| | | GGGSDIQMTQ SPSTLSASIG DRVTITCRAS EGIYHWLAWY QQKPGKAPKL |
| | | LIYKASSLAS GAPSRFSGSG SGTDFTLTIS SLQPDDFATY YCQQYSNYPL |
| | | TFGGGTKLEI KAAAGSGGSG ILVKQSPMLV AYDNAVNLSC KYSYNLFSRE |
| | | FRASLHKGLD SAVEVCVVYG NYSQQLQVYS KTGFNCDGKL GNESVTFYLQ |
| | | NLYVNQTDIY FCKIEVMYPP PYLDNEKSNG TIIHVKGKHL CPSPLFPGPS |
| | | KPFWVLVVVG GVLACYSLLV TVAFIIFWVR SKRSRLLHSD YMNMTPRRPG |
| | | PTRKHYQPYA PPRDFAAYRS KKVAKKPTNK APHPKQEPQE INFPDDLPGS |
| | | NTAAPVQETL HGCQPVTQED GKESRISVQE RQ |
| 232 | GM-CSF_HN1_ | MWLQSLLLLG TVACSISQVQ LVQSGAEVKR PGASVQVSCR ASGYSINTYY |
| | spCD28_ | MQWVRQAPGA GLEWMGVINP SGVTSYAQKF QGRVTLTNDT STNTVYMQLN |
| | CD28_CD40 | SLTSADTAVY YCARWALWGD FGMDVWGKGT LVTVSSGGGG SGGGGSGGGG |
| | | SDIQMTQSPS TLSASIGDRV TITCRASEGI YHWLAWYQQK PGKAPKLLIY |
| | | KASSLASGAP SRFSGSGSGT DFTLTISSLQ PDDFATYYCQ QYSNYPLTFG |
| | | GGTKLEIKAA AGSGGSGILV KQSPMLVAYD NAVNLSCKYS YNLFSREFRA |
| | | SLHKGLDSAV EVCVVYGNYS QQLQVYSKTG FNCDGKLGNE SVTFYLQNLY |
| | | VNQTDIYFCK IEVMYPPPYL DNEKSNGTII HVKGKHLCPS PLFPGPSKPF |
| | | WVLVVVGGVL ACYSLLVTVA FIIFWVRSKR SRLLHSDYMN MTPRRPGPTR |
| | | KHYQPYAPPR DFAAYRSKKV AKKPTNKAPH PKQEPQEINF PDDLPGSNTA |
| | | APVQETLHGC QPVTQEDGKE SRISVQERQ |
| 233 | hIgGk-VIII_ | MEAPAQLLFL LLLWLPDTTR QVQLVQSGAE VKRPGASVQV SCRASGYSIN |
| | HN1_ | TYYMQWVRQA PGAGLEWMGV INPSGVTSYA QKFQGRVTLT NDTSTNTVYM |
| | spCD28_ | QLNSLTSADT AVYYCARWAL WGDFGMDVWG KGTLVTVSSG GGGSGGGGSG |
| | CD28_CD40 | GGGSDIQMTQ SPSTLSASIG DRVTITCRAS EGIYHWLAWY QQKPGKAPKL |
| | | LIYKASSLAS GAPSRFSGSG SGTDFTLTIS SLQPDDFATY YCQQYSNYPL |
| | | TFGGGTKLEI KAAAGSGGSG ILVKQSPMLV AYDNAVNLSC KYSYNLFSRE |
| | | FRASLHKGLD SAVEVCVVYG NYSQQLQVYS KTGFNCDGKL GNESVTFYLQ |
| | | NLYVNQTDIY FCKIEVMYPP PYLDNEKSNG TIIHVKGKHL CPSPLFPGPS |
| | | KPFWVLVVVG GVLACYSLLV TVAFIIFWVR SKRSRLLHSD YMNMTPRRPG |
| | | PTRKHYQPYA PPRDFAAYRS KKVAKKPTNK APHPKQEPQE INFPDDLPGS |
| | | NTAAPVQETL HGCQPVTQED GKESRISVQE RQ |
| 234 | OSM_HN1_spCD8_ | MGVLLTQRTL LSLVLALLFP SMASMQVQLV QSGAEVKRPG ASVQVSCRAS |
| | CD28_CD40 | GYSINTYYMQ WVRQAPGAGL EWMGVINPSG VTSYAQKFQG RVTLTNDTST |
| | CTP238 | NTVYMQLNSL TSADTAVYYC ARWALWGDFG MDVWGKGTLV TVSSGGGGSG |
| | | GGGSGGGGSD IQMTQSPSTL SASIGDRVTI TCRASEGIYH WLAWYQQKPG |
| | | KAPKLLIYKA SSLASGAPSR FSGSGSGTDF TLTISSLQPD DFATYYCQQY |
| | | SNYPLTFGGG TKLEIKAAAG SGGSGFVPVF LPAKPTTTPA PRPPTPAPTI |
| | | ASQPLSLRPE ACRPAAGGAV HTRGLDFACD IYIWAPLAGT CGVLLLSLVI |
| | | TLYCNHRNRS KRSRLLHSDY MNMTPRRPGP TRKHYQPYAP PRDFAAYRSK |
| | | KVAKKPTNKA PHPKQEPQEI NFPDDLPGSN TAAPVQETLH GCQPVTQEDG |
| | | KESRISVQER Q |
| 235 | CD8a_HN1_spCD8_ | MALPVTALLL PLALLLHAAR PQVQLVQSGA EVKRPGASVQ VSCRASGYSI |
| | CD28_CD40 | NTYYMQWVRQ APGAGLEWMG VINPSGVTSY AQKFQGRVTL TNDTSTNTVY |
| | | MQLNSLTSAD TAVYYCARWA LWGDFGMDVW GKGTLVTVSS GGGGSGGGGS |
| | | GGGGSDIQMT QSPSTLSASI GDRVTITCRA SEGIYHWLAW YQQKPGKAPK |
| | | LLIYKASSLA SGAPSRFSGS GSGTDFTLTI SSLQPDDFAT YYCQQYSNYP |
| | | LTFGGGTKLE IKAAAGSGGS GFVPVFLPAK PTTTPAPRPP TPAPTIASQP |
| | | LSLRPEACRP AAGGAVHTRG LDFACDIYIW APLAGTCGVL LLSLVITLYC |
| | | NHRNRSKRSR LLHSDYMNMT PRRPGPTRKH YQPYAPPRDF AAYRSKKVAK |
| | | KPTNKAPHPK QEPQEINFPD DLPGSNTAAP VQETLHGCQP VTQEDGKESR |
| | | ISVQERQ |
| 236 | CD2_HN1_spCD8_ | MSFPCKFVAS FLLIFNVSSK GAVSQVQLVQ SGAEVKRPGA SVQVSCRASG |
| | CD28_CD40 | YSINTYYMQW VRQAPGAGLE WMGVINPSGV TSYAQKFQGR VTLTNDTSTN |
| | | TVYMQLNSLT SADTAVYYGA RWALWGDFGM DVWGKGTLVT VSSGGGGSGG |
| | | GGSGGGGSDI QMTQSPSTLS ASIGDRVTIT CRASEGIYHW LAWYQQKPGK |
| | | APKLLIYKAS SLASGAPSRF SGSGSGTDFT LTISSLQPDD FATYYCQQYS |
| | | NYPLTFGGGT KLEIKAAAGS GGSGFVPVFL PAKPTTTPAP RPPTPAPTIA |
| | | SQPLSLRPEA CRPAAGGAVH TRGLDFACDI YIWAPLAGTC GVLLLSLVIT |
| | | LYCNHRNRSK RSRLLHSDYM NMTPRRPGPT RKHYQPYAPP RDFAAYRSKK |
| | | VAKKPTNKAP HPKQEPQEIN FPDDLPGSNT AAPVQETLHG CQPVTQEDGK |
| | | ESRISVQERQ |
| 237 | IL2_HN1_spCD8_ | MYRMQLLSCI ALSLALVTNS QVQLVQSGAE VKRPGASVQV SCRASGYSIN |
| | CD28_CD40 | TYYMQWVRQA PGAGLEWMGV INPSGVTSYA QKFQGRVTLT NDTSTNTVYM |
| | | QLNSLTSADT AVYYCARWAL WGDFGMDVWG KGTLVTVSSG GGGSGGGGSG |
| | | GGGSDIQMTQ SPSTLSASIG DRVTITCRAS EGIYHWLAWY QQKPGKAPKL |
| | | LIYKASSLAS GAPSRFSGSG SGTDFTLTIS SLQPDDFATY YCQQYSNYPL |
| | | TFGGGTKLEI KAAAGSGGSG FVPVFLPAKP TTTPAPRPPT PAPTIASQPL |
| | | SLRPEACRPA AGGAVHTRGL DFACDIYIWA PLAGTCGVLL LSLVITLYCN |
| | | HRNRSKRSRL LHSDYMNMTP RRPGPTRKHY QPYAPPRDFA AYRSKKVAKK |
| | | PTNKAPHPKQ EPQEINFPDD LPGSNTAAPV QETLHGCQPV TQEDGKESRI |
| | | SVQERQ |
| 238 | GM-CSF_HN1_ | MWLQSLLLLG TVACSISQVQ LVQSGAEVKR PGASVQVSCR ASGYSINTYY |
| | spCD8_ | MQWVRQAPGA GLEWMGVINP SGVTSYAQKF QGRVTLTNDT STNTVYMQLN |
| | CD28_CD40 | SLTSADTAVY YCARWALWGD FGMDVWGKGT LVTVSSGGGG SGGGGSGGGG |
| | | SDIQMTQSPS TLSASIGDRV TITCRASEGI YHWLAWYQQK PGKAPKLLIY |
| | | KASSLASGAP SRFSGSGSGT DFTLTISSLQ PDDFATYYCQ QYSNYPLTFG |
| | | GGTKLEIKAA AGSGGSGFVP VFLPAKPTTT PAPRPPTPAP TIASQPLSLR |
| | | PEACRPAAGG AVHTRGLDFA CDIYIWAPLA GTCGVLLLSL VITLYCNHRN |
| | | RSKRSRLLHS DYMNMTPRRP GPTRKHYQPY APPRDFAAYR SKKVAKKPTN |
| | | KAPHPKQEPQ EINFPDDLPG SNTAAPVQET LHGCQPVTQE DGKESRISVQ |
| | | ERQ |
| 239 | hIgGk-VIII_ | MEAPAQLLFL LLLWLPDTTR QVQLVQSGAE VKRPGASVQV SCRASGYSIN |
| | HN1_spCD8_ | TYYMQWVRQA PGAGLEWMGV INPSGVTSYA QKFQGRVTLT NDTSTNTVYM |
| | CD28_CD40 | QLNSLTSADT AVYYCARWAL WGDFGMDVWG KGTLVTVSSG GGGSGGGGSG |
| | | GGGSDIQMTQ SPSTLSASIG DRVTITCRAS EGIYHWLAWY QQKPGKAPKL |
| | | LIYKASSLAS GAPSRFSGSG SGTDFTLTIS SLQPDDFATY YCQQYSNYPL |
| | | TFGGGTKLEI KAAAGSGGSG FVPVFLPAKP TTTPAPRPPT PAPTIASQPL |
| | | SLRPEACRPA AGGAVHTRGL DFACDIYIWA PLAGTCGVLL LSLVITLYCN |
| | | HRNRSKRSRL LHSDYMNMTP RRPGPTRKHY QPYAPPRDFA AYRSKKVAKK |
| | | PTNKAPHPKQ EPQEINFPDD LPGSNTAAPV QETLHGCQPV TQEDGKESRI |
| | | SVQERQ |
| 240 | OSM_HN1_ | MGVLLTQRTL LSLVLALLFP SMASMQVQLV QSGAEVKRPG ASVQVSCRAS |
| | CD28TM_CD28_ | GYSINTYYMQ WVRQAPGAGL EWMGVINPSG VTSYAQKFQG RVTLTNDTST |
| | CD40 | NTVYMQLNSL TSADTAVYYC ARWALWGDFG MDVWGKGTLV TVSSGGGGSG |
| | | GGGSGGGGSD IQMTQSPSTL SASIGDRVTI TGRASEGIYH WLAWYQQKPG |
| | | KAPKLLIYKA SSLASGAPSR FSGSGSGTDF TLTISSLQPD DFATYYCQQY |
| | | SNYPLTFGGG TKLEIKAAAG SGGSGFWVLV VVGGVLACYS LLVTVAFIIF |
| | | WVRSKRSRLL HSDYMNMTPR RPGPTRKHYQ PYAPPRDFAA YRSKKVAKKP |
| | | TNKAPHPKQE PQEINFPDDL PGSNTAAPVQ ETLHGCQPVT QEDGKESRIS |
| | | VQERQRSKRS RLLHSDYMNM TPRRPGPTRK HYQPYAPPRD FAAYRSKKVA |
| | | KKPTNKAPHP KQEPQEINFP DDLPGSNTAA PVQETLHGCQ PVTQEDGKES |
| | | RISVQERQ |
| 241 | CD8a_HN1_ | MALPVTALLL PLALLLHAAR PQVQLVQSGA EVKRPGASVQ VSCRASGYSI |
| | CD28TM_CD28_ | NTYYMQWVRQ APGAGLEWMG VINPSGVTSY AQKFQGRVTL TNDTSTNTVY |
| | CD40 | MQLNSLTSAD TAVYYCARWA LWGDFGMDVW GKGTLVTVSS GGGGSGGGGS |
| | | GGGGSDIQMT QSPSTLSASI GDRVTITCRA SEGIYHWLAW YQQKPGKAPK |
| | | LLIYKASSLA SGAPSRFSGS GSGTDFTLTI SSLQPDDFAT YYCQQYSNYP |
| | | LTFGGGTKLE IKAAAGSGGS GFWVLVVVGG VLACYSLLVT VAFIIFWVRS |
| | | KRSRLLHSDY MNMTPRRPGP TRKHYQPYAP PRDFAAYRSK KVAKKPTNKA |
| | | PHPKQEPQEI NFPDDLPGSN TAAPVQETLH GCQPVTQEDG KESRISVQER |
| | | Q |
| 242 | CD2_HN1_ | MSFPCKFVAS FLLIFNVSSK GAVSQVQLVQ SGAEVKRPGA SVQVSCRASG |
| | CD28TM_CD28_ | YSINTYYMQW VRQAPGAGLE WMGVINPSGV TSYAQKFQGR VTLTNDTSTN |
| | CD40 | TVYMQLNSLT SADTAVYYCA RWALWGDFGM DVWGKGTLVT VSSGGGGSGG |
| | | GGSGGGGSDI QMTQSPSTLS ASIGDRVTIT CRASEGIYHW LAWYQQKPGK |
| | | APKLLIYKAS SLASGAPSRF SGSGSGTDFT LTISSLQPDD FATYYCQQYS |
| | | NYPLTFGGGT KLEIKAAAGS GGSGFWVLVV VGGVLACYSL LVTVAFIIFW |
| | | VRSKRSRLLH SDYMNMTPRR PGPTRKHYQP YAPPRDFAAY RSKKVAKKPT |
| | | NKAPHPKQEP QEINFPDDLP GSNTAAPVQE TLHGCQPVTQ EDGKESRISV |
| | | QERQ |
| 243 | IL2_HN1_ | MYRMQLLSCI ALSLALVTNS QVQLVQSGAE VKRPGASVQV SCRASGYSIN |
| | CD28TM_CD28_ | TYYMQWVRQA PGAGLEWMGV INPSGVTSYA QKFQGRVTLT NDTSTNTVYM |
| | CD40 | QLNSLTSADT AVYYCARWAL WGDFGMDVWG KGTLVTVSSG GGGSGGGGSG |
| | | GGGSDIQMTQ SPSTLSASIG DRVTITCRAS EGIYHWLAWY QQKPGKAPKL |
| | | LIYKASSLAS GAPSRFSGSG SGTDFTLTIS SLQPDDFATY YCQQYSNYPL |
| | | TFGGGTKLEI KAAAGSGGSG FWVLVVVGGV LACYSLLVTV AFIIFWVRSK |
| | | RSRLLHSDYM NMTPRRPGPT RKHYQPYAPP RDFAAYRSKK VAKKPTNKAP |
| | | HPKQEPQEIN FPDDLPGSNT AAPVQETLHG CQPVTQEDGK ESRISVQERQ |
| 244 | GM-CSF_HN1_ | MWLQSLLLLG TVACSISQVQ LVQSGAEVKR PGASVQVSCR ASGYSINTYY |
| | CD28TM_ | MQWVRQAPGA GLEWMGVINP SGVTSYAQKF QGRVTLTNDT STNTVYMQLN |
| | CD28_CD40 | SLTSADTAVY YCARWALWGD FGMDVWGKGT LVTVSSGGGG SGGGGSGGGG |
| | | SDIQMTQSPS TLSASIGDRV TITCRASEGI YHWLAWYQQK PGKAPKLLIY |
| | | KASSLASGAP SRFSGSGSGT DFTLTISSLQ PDDFATYYCQ QYSNYPLTFG |
| | | GGTKLEIKAA AGSGGSGFWV LVVVGGVLAC YSLLVTVAFI IFWVRSKRSR |
| | | LLHSDYMNMT PRRPGPTRKH YQPYAPPRDF AAYRSKKVAK KPTNKAPHPK |
| | | QEPQEINFPD DLPGSNTAAP VQETLHGCQP VTQEDGKESR ISVQERQ |
| 245 | hIgGk-VIII_ | MEAPAQLLFL LLLWLPDTTR QVQLVQSGAE VKRPGASVQV SCRASGYSIN |
| | HN1_CD28TM_ | TYYMQWVRQA PGAGLEWMGV INPSGVTSYA QKFQGRVTLT NDTSTNTVYM |
| | CD28_CD40 | QLNSLTSADT AVYYCARWAL WGDFGMDVWG KGTLVTVSSG GGGSGGGGSG |
| | | GGGSDIQMTQ SPSTLSASIG DRVTITCRAS EGIYHWLAWY QQKPGKAPKL |
| | | LIYKASSLAS GAPSRFSGSG SGTDFTLTIS SLQPDDFATY YCQQYSNYPL |
| | | TFGGGTKLEI KAAAGSGGSG FWVLVVVGGV LACYSLLVTV AFIIFWVRSK |
| | | RSRLLHSDYM NMTPRRPGPT RKHYQPYAPP RDFAAYRSKK VAKKPTNKAP |
| | | HPKQEPQEIN FPDDLPGSNT AAPVQETLHG CQPVTQEDGK ESRISVQERQ |
| 246 | OSM_M912_ | MGVLLTQRTL LSLVLALLFP SMASMQVQLQ ESGPGLVKPS ETLSLTCTVS |
| | spCD28_ | GGSVSSGSYY WSWIRQPPGK GLEWIGYIYY SGSTNYNPSL KSRVTISVDT |
| | CD28_CD40 | SKNQFSLKLS SVTAADTAVY YCAREGKNGA FDIWGQGTMV TVSSGGGGSG |
| | CTP227 | GGGSGGGGSD IQMTQSPSSL SASVGDRVTI TCRASQSISS YLNWYQQKPG |
| | | KAPKLLIYAA SSLQSGVPSG FSGSGSGTDF TLTISSLQPE DFATYYCQQS |
| | | YSTPLTFGGG TKVEIKAAAG SGGSGILVKQ SPMLVAYDNA VNLSCKYSYN |
| | | LFSREFRASL HKGLDSAVEV CVVYGNYSQQ LQVYSKTGFN CDGKLGNESV |
| | | TFYLQNLYVN QTDIYFCKIE VMYPPPYLDN EKSNGTIIHV KGKHLCPSPL |
| | | FPGPSKPFWV LVVVGGVLAC YSLLVTVAFI IFWVRSKRSR LLHSDYMNMT |
| | | PRRPGPTRKH YQPYAPPRDF AAYRSKKVAK KPTNKAPHPK QEPQEINFPD |
| | | DLPGSNTAAP VQETLHGCQP VTQEDGKESR ISVQERQ |
| 247 | CD8a_M912_ | MALPVTALLL PLALLLHAAR PQVQLQESGP GLVKPSETLS LTCTVSGGSV |
| | spCD28_ | SSGSYYWSWI RQPPGKGLEW IGYIYYSGST NYNPSLKSRV TISVDTSKNQ |
| | CD28_CD40 | FSLKLSSVTA ADTAVYYCAR EGKNGAFDIW GQGTMVTVSS GGGGSGGGGS |
| | | GGGGSDIQMT QSPSSLSASV GDRVTITCRA SQSISSYLNW YQQKPGKAPK |
| | | LLIYAASSLQ SGVPSGFSGS GSGTDFTLTI SSLQPEDFAT YYCQQSYSTP |
| | | LTFGGGTKVE IKAAAGSGGS GILVKQSPML VAYDNAVNLS CKYSYNLFSR |
| | | EFRASLHKGL DSAVEVCVVY GNYSQQLQVY SKTGFNCDGK LGNESVTFYL |
| | | QNLYVNQTDI YFCKIEVMYP PPYLDNEKSN GTIIHVKGKH LCPSPLFPGP |
| | | SKPFWVLVVV GGVLACYSLL VTVAFIIFWV RSKRSRLLHS DYMNMTPRRP |
| | | GPTRKHYQPY APPRDFAAYR SKKVAKKPTN KAPHPKQEPQ EINFPDDLPG |
| | | SNTAAPVQET LHGCQPVTQE DGKESRISVQ ERQ |
| 248 | CD2_M912_ | MSFPCKFVAS FLLIFNVSSK GAVSQVQLQE SGPGLVKPSE TLSLTCTVSG |
| | spCD28_ | GSVSSGSYYW SWIRQPPGKG LEWIGYIYYS GSTNYNPSLK SRVTISVDTS |
| | CD28_CD40 | KNQFSLKLSS VTAADTAVYY CAREGKNGAF DIWGQGTMVT VSSGGGGSGG |
| | | GGSGGGGSDI QMTQSPSSLS ASVGDRVTIT CRASQSISSY LNWYQQKPGK |
| | | APKLLIYAAS SLQSGVPSGF SGSGSGTDFT LTISSLQPED FATYYCQQSY |
| | | STPLTFGGGT KVEIKAAAGS GGSGILVKQS PMLVAYDNAV NLSCKYSYNL |
| | | FSREFRASLH KGLDSAVEVC VVYGNYSQQL QVYSKTGFNC DGKLGNESVT |
| | | FYLQNLYVNQ TDIYFCKIEV MYPPPYLDNE KSNGTIIHVK GKHLCPSPLF |
| | | PGPSKPFWVL VVVGGVLACY SLLVTVAFII FWVRSKRSRL LHSDYMNMTP |
| | | RRPGPTRKHY QPYAPPRDFA AYRSKKVAKK PTNKAPHPKQ EPQEINFPDD |
| | | LPGSNTAAPV QETLHGCQPV TQEDGKESRI SVQERQ |
| 249 | IL2_M912_ | MYRMQLLSCI ALSLALVTNS QVQLQESGPG LVKPSETLSL TCTVSGGSVS |
| | spCD28_ | SGSYYWSWIR QPPGKGLEWI GYIYYSGSTN YNPSLKSRVT ISVDTSKNQF |
| | CD28_CD40 | SLKLSSVTAA DTAVYYCARE GKNGAFDIWG QGTMVTVSSG GGGSGGGGSG |
| | | GGGSDIQMTQ SPSSLSASVG DRVTITCRAS QSISSYLNWY QQKPGKAPKL |
| | | LIYAASSLQS GVPSGFSGSG SGTDFTLTIS SLQPEDFATY YCQQSYSTPL |
| | | TFGGGTKVEI KAAAGSGGSG ILVKQSPMLV AYDNAVNLSC KYSYNLFSRE |
| | | FRASLHKGLD SAVEVCVVYG NYSQQLQVYS KTGFNCDGKL GNESVTFYLQ |
| | | NLYVNQTDIY FCKIEVMYPP PYLDNEKSNG TIIHVKGKHL CPSPLFPGPS |
| | | KPFWVLVVVG GVLACYSLLV TVAFIIFWVR SKRSRLLHSD YMNMTPRRPG |
| | | PTRKHYQPYA PPRDFAAYRS KKVAKKPTNK APHPKQEPQE INFPDDLPGS |
| | | NTAAPVQETL HGCQPVTQED GKESRISVQE RQ |
| 250 | GM-CSF_M912_ | MWLQSLLLLG TVACSISQVQ LQESGPGLVK PSETLSLTCT VSGGSVSSGS |
| | spCD28_ | YYWSWIRQPP GKGLEWIGYI YYSGSTNYNP SLKSRVTISV DTSKNQFSLK |
| | CD28_CD40 | LSSVTAADTA VYYCAREGKN GAFDIWGQGT MVTVSSGGGG SGGGGSGGGG |
| | | SDIQMTQSPS SLSASVGDRV TITCRASQSI SSYLNWYQQK PGKAPKLLIY |
| | | AASSLQSGVP SGFSGSGSGT DFTLTISSLQ PEDFATYYCQ QSYSTPLTFG |
| | | GGTKVEIKAA AGSGGSGILV KQSPMLVAYD NAVNLSCKYS YNLFSREFRA |
| | | SLHKGLDSAV EVCVVYGNYS QQLQVYSKTG FNCDGKLGNE SVTFYLQNLY |
| | | VNQTDIYFCK IEVMYPPPYL DNEKSNGTII HVKGKHLCPS PLFPGPSKPF |
| | | WVLVVVGGVL ACYSLLVTVA FIIFWVRSKR SRLLHSDYMN MTPRRPGPTR |
| | | KHYQPYAPPR DFAAYRSKKV AKKPTNKAPH PKQEPQEINF PDDLPGSNTA |
| | | APVQETLHGC QPVTQEDGKE SRISVQERQ |
| 251 | hIgGk-VIII_ | MEAPAQLLFL LLLWLPDTTR QVQLQESGPG LVKPSETLSL TCTVSGGSVS |
| | M912_ | SGSYYWSWIR QPPGKGLEWI GYIYYSGSTN YNPSLKSRVT ISVDTSKNQF |
| | spCD28_ | SLKLSSVTAA DTAVYYCARE GKNGAFDIWG QGTMVTVSSG GGGSGGGGSG |
| | CD28_CD40 | GGGSDIQMTQ SPSSLSASVG DRVTITCRAS QSISSYLNWY QQKPGKAPKL |
| | | LIYAASSLQS GVPSGFSGSG SGTDFTLTIS SLQPEDFATY YCQQSYSTPL |
| | | TFGGGTKVEI KAAAGSGGSG ILVKQSPMLV AYDNAVNLSC KYSYNLFSRE |
| | | FRASLHKGLD SAVEVCVVYG NYSQQLQVYS KTGFNCDGKL GNESVTFYLQ |
| | | NLYVNQTDIY FCKIEVMYPP PYLDNEKSNG TIIHVKGKHL CPSPLFPGPS |
| | | KPFWVLVVVG GVLACYSLLV TVAFIIFWVR SKRSRLLHSD YMNMTPRRPG |
| | | PTRKHYQPYA PPRDFAAYRS KKVAKKPTNK APHPKQEPQE INFPDDLPGS |
| | | NTAAPVQETL HGCQPVTQED GKESRISVQE RQ |
| 252 | OSM_M912_ | MGVLLTQRTL LSLVLALLFP SMASMQVQLQ ESGPGLVKPS ETLSLTCTVS |
| | spCD8_CD28_CD40 | GGSVSSGSYY WSWIRQPPGK GLEWIGYIYY SGSTNYNPSL KSRVTISVDT |
| | CTP239 | SKNQFSLKLS SVTAADTAVY YCAREGKNGA FDIWGQGTMV TVSSGGGGSG |
| | | GGGSGGGGSD IQMTQSPSSL SASVGDRVTI TCRASQSISS YLNWYQQKPG |
| | | KAPKLLIYAA SSLQSGVPSG FSGSGSGTDF TLTISSLQPE DFATYYCQQS |
| | | YSTPLTFGGG TKVEIKAAAG SGGSGFVPVF LPAKPTTTPA PRPPTPAPTI |
| | | ASQPLSLRPE ACRPAAGGAV HTRGLDFACD IYIWAPLAGT CGVLLLSLVI |
| | | TLYCNHRNRS KRSRLLHSDY MNMTPRRPGP TRKHYQPYAP PRDFAAYRSK |
| | | KVAKKPTNKA PHPKQEPQEI NFPDDLPGSN TAAPVQETLH GCQPVTQEDG |
| | | KESRISVQER Q |
| 253 | CD8a_M912_ | MALPVTALLL PLALLLHAAR PQVQLQESGP GLVKPSETLS LTCTVSGGSV |
| | spCD8_CD28_CD40 | SSGSYYWSWI RQPPGKGLEW IGYIYYSGST NYNPSLKSRV TISVDTSKNQ |
| | | FSLKLSSVTA ADTAVYYCAR EGKNGAFDIW GQGTMVTVSS GGGGSGGGGS |
| | | GGGGSDIQMT QSPSSLSASV GDRVTITCRA SQSISSYLNW YQQKPGKAPK |
| | | LLIYAASSLQ SGVPSGFSGS GSGTDFTLTI SSLQPEDFAT YYCQQSYSTP |
| | | LTFGGGTKVE IKAAAGSGGS GFVPVFLPAK PTTTPAPRPP TPAPTIASQP |
| | | LSLRPEACRP AAGGAVHTRG LDFACDIYIW APLAGTCGVL LLSLVITLYC |
| | | NHRNRSKRSR LLHSDYMNMT PRRPGPTRKH YQPYAPPRDF AAYRSKKVAK |
| | | KPTNKAPHPK QEPQEINFPD DLPGSNTAAP VQETLHGCQP VTQEDGKESR |
| | | ISVQERQ |
| 254 | CD2_M912_spCD8_ | MSFPCKFVAS FLLIFNVSSK GAVSQVQLQE SGPGLVKPSE TLSLTCTVSG |
| | CD28_CD40 | GSVSSGSYYW SWIRQPPGKG LEWIGYIYYS GSTNYNPSLK SRVTISVDTS |
| | | KNQFSLKLSS VTAADTAVYY CAREGKNGAF DIWGQGTMVT VSSGGGGSGG |
| | | GGSGGGGSDI QMTQSPSSLS ASVGDRVTIT CRASQSISSY LNWYQQKPGK |
| | | APKLLIYAAS SLQSGVPSGF SGSGSGTDFT LTISSLQPED FATYYCQQSY |
| | | STPLTFGGGT KVEIKAAAGS GGSGFVPVFL PAKPTTTPAP RPPTPAPTIA |
| | | SQPLSLRPEA CRPAAGGAVH TRGLDFACDI YIWAPLAGTC GVLLLSLVIT |
| | | LYCNHRNRSK RSRLLHSDYM NMTPRRPGPT RKHYQPYAPP RDFAAYRSKK |
| | | VAKKPTNKAP HPKQEPQEIN FPDDLPGSNT AAPVQETLHG CQPVTQEDGK |
| | | ESRISVQERQ |
| 255 | IL2_M912_spCD8_ | MYRMQLLSCI ALSLALVTNS QVQLQESGPG LVKPSETLSL TCTVSGGSVS |
| | CD28_CD40 | SGSYYWSWIR QPPGKGLEWI GYIYYSGSTN YNPSLKSRVT ISVDTSKNQF |
| | | SLKLSSVTAA DTAVYYCARE GKNGAFDIWG QGTMVTVSSG GGGSGGGGSG |
| | | GGGSDIQMTQ SPSSLSASVG DRVTITCRAS QSISSYLNWY QQKPGKAPKL |
| | | LIYAASSLQS GVPSGFSGSG SGTDFTLTIS SLQPEDFATY YCQQSYSTPL |
| | | TFGGGTKVEI KAAAGSGGSG FVPVFLPAKP TTTPAPRPPT PAPTIASQPL |
| | | SLRPEACRPA AGGAVHTRGL DFACDIYIWA PLAGTCGVLL LSLVITLYCN |
| | | HRNRSKRSRL LHSDYMNMTP RRPGPTRKHY QPYAPPRDFA AYRSKKVAKK |
| | | PTNKAPHPKQ EPQEINFPDD LPGSNTAAPV QETLHGCQPV TQEDGKESRI |
| | | SVQERQ |
| 256 | GM-CSF_ | MWLQSLLLLG TVACSISQVQ LQESGPGLVK PSETLSLTCT VSGGSVSSGS |
| | M912_spCD8_ | YYWSWIRQPP GKGLEWIGYI YYSGSTNYNP SLKSRVTISV DTSKNQFSLK |
| | CD28_CD40 | LSSVTAADTA VYYCAREGKN GAFDIWGQGT MVTVSSGGGG SGGGGSGGGG |
| | | SDIQMTQSPS SLSASVGDRV TITCRASQSI SSYLNWYQQK PGKAPKLLIY |
| | | AASSLQSGVP SGFSGSGSGT DFTLTISSLQ PEDFATYYCQ QSYSTPLTFG |
| | | GGTKVEIKAA AGSGGSGFVP VFLPAKPTTT PAPRPPTPAP TIASQPLSLR |
| | | PEACRPAAGG AVHTRGLDFA CDIYIWAPLA GTCGVLLLSL VITLYCNHRN |
| | | RSKRSRLLHS DYMNMTPRRP GPTRKHYQPY APPRDFAAYR SKKVAKKPTN |
| | | KAPHPKQEPQ EINFPDDLPG SNTAAPVQET LHGCQPVTQE DGKESRISVQ |
| | | ERQ |
| 257 | hIgGk-VIII_ | MEAPAQLLFL LLLWLPDTTR QVQLQESGPG LVKPSETLSL TCTVSGGSVS |
| | M912_spCD8_ | SGSYYWSWIR QPPGKGLEWI GYIYYSGSTN YNPSLKSRVT ISVDTSKNQF |
| | CD28_CD40 | SLKLSSVTAA DTAVYYCARE GKNGAFDIWG QGTMVTVSSG GGGSGGGGSG |
| | | GGGSDIQMTQ SPSSLSASVG DRVTITCRAS QSISSYLNWY QQKPGKAPKL |
| | | LIYAASSLQS GVPSGFSGSG SGTDFTLTIS SLQPEDFATY YCQQSYSTPL |
| | | TFGGGTKVEI KAAAGSGGSG FVPVFLPAKP TTTPAPRPPT PAPTIASQPL |
| | | SLRPEACRPA AGGAVHTRGL DFACDIYIWA PLAGTCGVLL LSLVITLYCN |
| | | HRNRSKRSRL LHSDYMNMTP RRPGPTRKHY QPYAPPRDFA AYRSKKVAKK |
| | | PTNKAPHPKQ EPQEINFPDD LPGSNTAAPV QETLHGCQPV TQEDGKESRI |
| | | SVQERQ |
| 258 | OSM_M912_ | MGVLLTQRTL LSLVLALLFP SMASMQVOLQ ESGPGLVKPS ETLSLTCTVS |
| | CD28TM_ | GGSVSSGSYY WSWIRQPPGK GLEWIGYIYY SGSTNYNPSL KSRVTISVDT |
| | CD28_CD40 | SKNQFSLKLS SVTAADTAVY YCAREGKNGA FDIWGQGTMV TVSSGGGGSG |
| | | GGGSGGGGSD IQMTQSPSSL SASVGDRVTI TCRASQSISS YLNWYQQKPG |
| | | KAPKLLIYAA SSLQSGVPSG FSGSGSGTDF TLTISSLQPE DFATYYCQQS |
| | | YSTPLTFGGG TKVEIKAAAG SGGSGFWVLV VVGGVLACYS LLVTVAFIIF |
| | | WVRSKRSRLL HSDYMNMTPR RPGPTRKHYQ PYAPPRDFAA YRSKKVAKKP |
| | | TNKAPHPKQE PQEINFPDDL PGSNTAAPVQ ETLHGCQPVT QEDGKESRIS |
| | | VQERQ |
| 259 | CD8a_M912_ | MALPVTALLL PLALLLHAAR PQVQLQESGP GLVKPSETLS LTCTVSGGSV |
| | CD28TM_ | SSGSYYWSWI RQPPGKGLEW IGYIYYSGST NYNPSLKSRV TISVDTSKNQ |
| | CD28_CD40 | FSLKLSSVTA ADTAVYYCAR EGKNGAFDIW GQGTMVTVSS GGGGSGGGGS |
| | | GGGGSDIQMT QSPSSLSASV GDRVTITCRA SQSISSYLNW YQQKPGKAPK |
| | | LLIYAASSLQ SGVPSGFSGS GSGTDFTLTI SSLQPEDFAT YYCQQSYSTP |
| | | LTFGGGTKVE IKAAAGSGGS GFWVLVVVGG VLACYSLLVT VAFIIFWVRS |
| | | KRSRLLHSDY MNMTPRRPGP TRKHYQPYAP PRDFAAYRSK KVAKKPTNKA |
| | | PHPKQEPQEI NFPDDLPGSN TAAPVQETLH GCQPVTQEDG KESRISVQER |
| | | Q |
| 260 | CD2_M912_ | MSFPCKFVAS FLLIFNVSSK GAVSQVQLQE SGPGLVKPSE TLSLTCTVSG |
| | CD28TM_ | GSVSSGSYYW SWIRQPPGKG LEWIGYIYYS GSTNYNPSLK SRVTISVDTS |
| | CD28_CD40 | KNQFSLKLSS VTAADTAVYY CAREGKNGAF DIWGQGTMVT VSSGGGGSGG |
| | | GGSGGGGSDI QMTQSPSSLS ASVGDRVTIT CRASQSISSY LNWYQQKPGK |
| | | APKLLIYAAS SLQSGVPSGF SGSGSGTDFT LTISSLQPED FATYYCQQSY |
| | | STPLTFGGGT KVEIKAAAGS GGSGFWVLVV VGGVLACYSL LVTVAFIIFW |
| | | VRSKRSRLLH SDYMNMTPRR PGPTRKHYQP YAPPRDFAAY RSKKVAKKPT |
| | | NKAPHPKQEP QEINFPDDLP GSNTAAPVQE TLHGCQPVTQ EDGKESRISV |
| | | QERQ |
| 261 | IL2_M912_ | MYRMQLLSCI ALSLALVTNS QVQLQESGPG LVKPSETLSL TCTVSGGSVS |
| | CD28TM_ | SGSYYWSWIR QPPGKGLEWI GYIYYSGSTN YNPSLKSRVT ISVDTSKNQF |
| | CD28_CD40 | SLKLSSVTAA DTAVYYCARE GKNGAFDIWG QGTMVTVSSG GGGSGGGGSG |
| | | GGGSDIQMTQ SPSSLSASVG DRVTITCRAS QSISSYLNWY QQKPGKAPKL |
| | | LIYAASSLQS GVPSGFSGSG SGTDFTLTIS SLQPEDFATY YCQQSYSTPL |
| | | TFGGGTKVEI KAAAGSGGSG FWVLVVVGGV LACYSLLVTV AFIIFWVRSK |
| | | RSRLLHSDYM NMTPRRPGPT RKHYQPYAPP RDFAAYRSKK VAKKPTNKAP |
| | | HPKQEPQEIN FPDDLPGSNT AAPVQETLHG CQPVTQEDGK ESRISVQERQ |
| 262 | GM-CSF_M912_ | MWLQSLLLLG TVACSISQVQ LQESGPGLVK PSETLSLTCT VSGGSVSSGS |
| | CD28TM_ | YYWSWIRQPP GKGLEWIGYI YYSGSTNYNP SLKSRVTISV DTSKNQFSLK |
| | CD28_CD40 | LSSVTAADTA VYYCAREGKN GAFDIWGQGT MVTVSSGGGG SGGGGSGGGG |
| | | SDIQMTQSPS SLSASVGDRV TITCRASQSI SSYLNWYQQK PGKAPKLLIY |
| | | AASSLQSGVP SGFSGSGSGT DFTLTISSLQ PEDFATYYCQ QSYSTPLTFG |
| | | GGTKVEIKAA AGSGGSGFWV LVVVGGVLAC YSLLVTVAFI IFWVRSKRSR |
| | | LLHSDYMNMT PRRPGPTRKH YQPYAPPRDF AAYRSKKVAK KPTNKAPHPK |
| | | QEPQEINFPD DLPGSNTAAP VQETLHGCQP VTQEDGKESR ISVQERQ |
| 263 | hIgGk-VIII_ | MEAPAQLLFL LLLWLPDTTR QVQLQESGPG LVKPSETLSL TCTVSGGSVS |
| | M912_ | SGSYYWSWIR QPPGKGLEWI GYIYYSGSTN YNPSLKSRVT ISVDTSKNQF |
| | CD28TM_ | SLKLSSVTAA DTAVYYCARE GKNGAFDIWG QGTMVTVSSG GGGSGGGGSG |
| | CD28_CD40 | GGGSDIQMTQ SPSSLSASVG DRVTITCRAS QSISSYLNWY QQKPGKAPKL |
| | | LIYAASSLQS GVPSGFSGSG SGTDFTLTIS SLQPEDFATY YCQQSYSTPL |
| | | TFGGGTKVEI KAAAGSGGSG FWVLVVVGGV LACYSLLVTV AFIIFWVRSK |
| | | RSRLLHSDYM NMTPRRPGPT RKHYQPYAPP RDFAAYRSKK VAKKPTNKAP |
| | | HPKQEPQEIN FPDDLPGSNT AAPVQETLHG CQPVTQEDGK ESRISVQERQ |
| 264 | OSM_HuYP218_ | MGVLLTQRTL LSLVLALLFP SMASMEVQLV ESGGGLVQPG GSLRLSCAAS |
| | spCD28_ | GFDLGFYFYA CWVRQAPGKG LEWVSCIYTA GSGSTYYASW AKGRFTISRD |
| | CD28_CD40 | NSKNTLYLQM NSLRAEDTAV YYCARSTANT RSTYYLNLWG QGTLVTVSSG |
| | CTP228 | GGGSGGGGSG GGGSDIQMTQ SPSSLSASVG DRVTITCQAS QRISSYLSWY |
| | | QQKPGKVPKL LIYGASTLAS GVPSRFSGSG SGTDFTLTIS SLQPEDVATY |
| | | YCQSYAYFDS NNWHAFGGGT KVEIAAAGSG GSGILVKQSP MLVAYDNAVN |
| | | LSCKYSYNLF SREFRASLHK GLDSAVEVCV VYGNYSQQLQ VYSKTGFNCD |
| | | GKLGNESVTF YLQNLYVNQT DIYFCKIEVM YPPPYLDNEK SNGTIIHVKG |
| | | KHLCPSPLFP GPSKPFWVLV VVGGVLACYS LLVTVAFIIF WVRSKRSRLL |
| | | HSDYMNMTPR RPGPTRKHYQ PYAPPRDFAA YRSKKVAKKP TNKAPHPKQE |
| | | PQEINFPDDL PGSNTAAPVQ ETLHGCQPVT QEDGKESRIS VQERQ |
| 265 | CD8a_HuYP218_ | MALPVTALLL PLALLLHAAR PEVQLVESGG GLVQPGGSLR LSCAASGFDL |
| | spCD28_ | GFYFYACWVR QAPGKGLEWV SCIYTAGSGS TYYASWAKGR FTISRDNSKN |
| | CD28_CD40 | TLYLQMNSLR AEDTAVYYGA RSTANTRSTY YLNLWGQGTL VTVSSGGGGS |
| | | GGGGSGGGGS DIQMTQSPSS LSASVGDRVT ITCQASQRIS SYLSWYQQKP |
| | | GKVPKLLIYG ASTLASGVPS RFSGSGSGTD FTLTISSLQP EDVATYYCQS |
| | | YAYFDSNNWH AFGGGTKVEI AAAGSGGSGI LVKQSPMLVA YDNAVNLSCK |
| | | YSYNLFSREF RASLHKGLDS AVEVCVVYGN YSQQLQVYSK TGFNCDGKLG |
| | | NESVTFYLQN LYVNQTDIYF CKIEVMYPPP YLDNEKSNGT IIHVKGKHLC |
| | | PSPLFPGPSK PFWVLVVVGG VLACYSLLVT VAFIIFWVRS KRSRLLHSDY |
| | | MNMTPRRPGP TRKHYQPYAP PRDFAAYRSK KVAKKPTNKA PHPKQEPQEI |
| | | NFPDDLPGSN TAAPVQETLH GCQPVTQEDG KESRISVQER Q |
| 266 | CD2_HuYP218_ | MSFPCKFVAS FLLIFNVSSK GAVSEVQLVE SGGGLVQPGG SLRLSCAASG |
| | spCD28_ | FDLGFYFYAC WVRQAPGKGL EWVSCIYTAG SGSTYYASWA KGRFTISRDN |
| | CD28_CD40 | SKNTLYLQMN SLRAEDTAVY YCARSTANTR STYYLNLWGQ GTLVTVSSGG |
| | | GGSGGGGSGG GGSDIQMTQS PSSLSASVGD RVTITCQASQ RISSYLSWYQ |
| | | QKPGKVPKLL IYGASTLASG VPSRFSGSGS GTDFTLTISS LQPEDVATYY |
| | | CQSYAYFDSN NWHAFGGGTK VEIAAAGSGG SGILVKQSPM LVAYDNAVNL |
| | | SCKYSYNLFS REFRASLHKG LDSAVEVCVV YGNYSQQLQV YSKTGFNCDG |
| | | KLGNESVTFY LQNLYVNQTD IYFCKIEVMY PPPYLDNEKS NGTIIHVKGK |
| | | HLCPSPLFPG PSKPFWVLVV VGGVLACYSL LVTVAFIIFW VRSKRSRLLH |
| | | SDYMNMTPRR PGPTRKHYQP YAPPRDFAAY RSKKVAKKPT NKAPHPKQEP |
| | | QEINFPDDLP GSNTAAPVQE TLHGCQPVTQ EDGKESRISV QERQ |
| 267 | IL2_HuYP218_ | MYRMQLLSCI ALSLALVTNS EVQLVESGGG LVQPGGSLRL SCAASGFDLG |
| | spCD28_ | FYFYACWVRQ APGKGLEWVS GIYTAGSGST YYASWAKGRF TISRDNSKNT |
| | CD28_CD40 | LYLQMNSLRA EDTAVYYCAR STANTRSTYY LNLWGQGTLV TVSSGGGGSG |
| | | GGGSGGGGSD IQMTQSPSSL SASVGDRVTI TCQASQRISS YLSWYQQKPG |
| | | KVPKLLIYGA STLASGVPSR FSGSGSGTDF TLTISSLQPE DVATYYCQSY |
| | | AYFDSNNWHA FGGGTKVEIA AAGSGGSGIL VKQSPMLVAY DNAVNLSCKY |
| | | SYNLFSREFR ASLHKGLDSA VEVCVVYGNY SQQLQVYSKT GFNCDGKLGN |
| | | ESVTFYLQNL YVNQTDIYFC KIEVMYPPPY LDNEKSNGTI IHVKGKHLCP |
| | | SPLFPGPSKP FWVLVVVGGV LACYSLLVTV AFIIFWVRSK RSRLLHSDYM |
| | | NMTPRRPGPT RKHYQPYAPP RDFAAYRSKK VAKKPTNKAP HPKQEPQEIN |
| | | FPDDLPGSNT AAPVQETLHG CQPVTQEDGK ESRISVQERQ |
| 268 | GM-CSF_HuYP218_ | MWLQSLLLLG TVACSISEVQ LVESGGGLVQ PGGSLRLSCA ASGFDLGFYF |
| | spCD28_ | YACWVRQAPG KGLEWVSCIY TAGSGSTYYA SWAKGRFTIS RDNSKNTLYL |
| | CD28_CD40 | QMNSLRAEDT AVYYCARSTA NTRSTYYLNL WGQGTLVTVS SGGGGSGGGG |
| | | SGGGGSDIQM TQSPSSLSAS VGDRVTITCQ ASQRISSYLS WYQQKPGKVP |
| | | KLLIYGASTL ASGVPSRFSG SGSGTDFTLT ISSLQPEDVA TYYCQSYAYF |
| | | DSNNWHAFGG GTKVEIAAAG SGGSGILVKQ SPMLVAYDNA VNLSCKYSYN |
| | | LFSREFRASL HKGLDSAVEV CVVYGNYSQQ LQVYSKTGFN CDGKLGNESV |
| | | TFYLQNLYVN QTDIYFCKIE VMYPPPYLDN EKSNGTIIHV KGKHLCPSPL |
| | | FPGPSKPFWV LVVVGGVLAC YSLLVTVAFI IFWVRSKRSR LLHSDYMNMT |
| | | PRRPGPTRKH YQPYAPPRDF AAYRSKKVAK KPTNKAPHPK QEPQEINFPD |
| | | DLPGSNTAAP VQETLHGCQP VTQEDGKESR ISVQERQ |
| 269 | hIgGk-VIII_ | MEAPAQLLFL LLLWLPDTTR EVQLVESGGG LVQPGGSLRL SCAASGFDLG |
| | HuYP218_ | FYFYACWVRQ APGKGLEWVS GIYTAGSGST YYASWAKGRF TISRDNSKNT |
| | spCD28_ | LYLQMNSLRA EDTAVYYCAR STANTRSTYY LNLWGQGTLV TVSSGGGGSG |
| | CD28_CD40 | GGGSGGGGSD IQMTQSPSSL SASVGDRVTI TCQASQRISS YLSWYQQKPG |
| | | KVPKLLIYGA STLASGVPSR FSGSGSGTDF TLTISSLQPE DVATYYCQSY |
| | | AYFDSNNWHA FGGGTKVEIA AAGSGGSGIL VKQSPMLVAY DNAVNLSCKY |
| | | SYNLFSREFR ASLHKGLDSA VEVCVVYGNY SQQLQVYSKT GFNCDGKLGN |
| | | ESVTFYLQNL YVNQTDIYFC KIEVMYPPPY LDNEKSNGTI IHVKGKHLCP |
| | | SPLFPGPSKP FWVLVVVGGV LACYSLLVTV AFIIFWVRSK RSRLLHSDYM |
| | | NMTPRRPGPT RKHYQPYAPP RDFAAYRSKK VAKKPTNKAP HPKQEPQEIN |
| | | FPDDLPGSNT AAPVQETLHG CQPVTQEDGK ESRISVQERQ |
| 270 | OSM_HuYP218_ | MGVLLTQRTL LSLVLALLFP SMASMEVQLV ESGGGLVQPG GSLRLSCAAS |
| | spCD8_ | GFDLGFYFYA CWVRQAPGKG LEWVSCIYTA GSGSTYYASW AKGRFTISRD |
| | CD28_CD40 | NSKNTLYLQM NSLRAEDTAV YYCARSTANT RSTYYLNLWG QGTLVTVSSG |
| | CTP240 | GGGSGGGGSG GGGSDIQMTQ SPSSLSASVG DRVTITCQAS QRISSYLSWY |
| | | QQKPGKVPKL LIYGASTLAS GVPSRFSGSG SGTDFTLTIS SLQPEDVATY |
| | | YCQSYAYFDS NNWHAFGGGT KVEIAAAGSG GSGFVPVFLP AKPTTTPAPR |
| | | PPTPAPTIAS QPLSLRPEAC RPAAGGAVHT RGLDFACDIY IWAPLAGTCG |
| | | VLLLSLVITL YCNHRNRSKR SRLLHSDYMN MTPRRPGPTR KHYQPYAPPR |
| | | DFAAYRSKKV AKKPTNKAPH PKQEPQEINF PDDLPGSNTA APVQETLHGC |
| | | QPVTQEDGKE SRISVQERQ |
| 271 | CD8a_HuYP218_ | MALPVTALLL PLALLLHAAR PEVQLVESGG GLVQPGGSLR LSCAASGFDL |
| | spCD8_ | GFYFYACWVR QAPGKGLEWV SCIYTAGSGS TYYASWAKGR FTISRDNSKN |
| | CD28_CD40 | TLYLQMNSLR AEDTAVYYCA RSTANTRSTY YLNLWGQGTL VTVSSGGGGS |
| | | GGGGSGGGGS DIQMTQSPSS LSASVGDRVT ITCQASQRIS SYLSWYQQKP |
| | | GKVPKLLIYG ASTLASGVPS RFSGSGSGTD FTLTISSLQP EDVATYYCQS |
| | | YAYFDSNNWH AFGGGTKVEI AAAGSGGSGF VPVFLPAKPT TTPAPRPPTP |
| | | APTIASQPLS LRPEACRPAA GGAVHTRGLD FACDIYIWAP LAGTCGVLLL |
| | | SLVITLYCNH RNRSKRSRLL HSDYMNMTPR RPGPTRKHYQ PYAPPRDFAA |
| | | YRSKKVAKKP TNKAPHPKQE PQEINFPDDL PGSNTAAPVQ ETLHGCQPVT |
| | | QEDGKESRIS VQERQ |
| 272 | CD2_HuYP218_ | MSFPCKFVAS FLLIFNVSSK GAVSEVQLVE SGGGLVQPGG SLRLSCAASG |
| | spCD8_ | FDLGFYFYAC WVRQAPGKGL EWVSCIYTAG SGSTYYASWA KGRFTISRDN |
| | CD28_CD40 | SKNTLYLQMN SLRAEDTAVY YCARSTANTR STYYLNLWGQ GTLVTVSSGG |
| | | GGSGGGGSGG GGSDIQMTQS PSSLSASVGD RVTITCQASQ RISSYLSWYQ |
| | | QKPGKVPKLL IYGASTLASG VPSRFSGSGS GTDFTLTISS LQPEDVATYY |
| | | CQSYAYFDSN NWHAFGGGTK VEIAAAGSGG SGFVPVFLPA KPTTTPAPRP |
| | | PTPAPTIASQ PLSLRPEACR PAAGGAVHTR GLDFACDIYI WAPLAGTCGV |
| | | LLLSLVITLY CNHRNRSKRS RLLHSDYMNM TPRRPGPTRK HYQPYAPPRD |
| | | FAAYRSKKVA KKPTNKAPHP KQEPQEINFP DDLPGSNTAA PVQETLHGCQ |
| | | PVTQEDGKES RISVQERQ |
| 273 | IL2_HuYP218_ | MYRMQLLSCI ALSLALVTNS EVQLVESGGG LVQPGGSLRL SCAASGFDLG |
| | spCD8_ | FYFYACWVRQ APGKGLEWVS GIYTAGSGST YYASWAKGRF TISRDNSKNT |
| | CD28_CD40 | LYLQMNSLRA EDTAVYYCAR STANTRSTYY LNLWGQGTLV TVSSGGGGSG |
| | | GGGSGGGGSD IQMTQSPSSL SASVGDRVTI TCQASQRISS YLSWYQQKPG |
| | | KVPKLLIYGA STLASGVPSR FSGSGSGTDF TLTISSLQPE DVATYYCQSY |
| | | AYFDSNNWHA FGGGTKVEIA AAGSGGSGFV PVFLPAKPTT TPAPRPPTPA |
| | | PTIASQPLSL RPEACRPAAG GAVHTRGLDF ACDIYIWAPL AGTCGVLLLS |
| | | LVITLYCNHR NRSKRSRLLH SDYMNMTPRR PGPTRKHYQP YAPPRDFAAY |
| | | RSKKVAKKPT NKAPHPKQEP QEINFPDDLP GSNTAAPVQE TLHGCQPVTQ |
| | | EDGKESRISV QERQ |
| 274 | GM-CSF_ | MWLQSLLLLG TVACSISEVQ LVESGGGLVQ PGGSLRLSCA ASGFDLGFYF |
| | HuYP218_ | YACWVRQAPG KGLEWVSCIY TAGSGSTYYA SWAKGRFTIS RDNSKNTLYL |
| | spCD8_ | QMNSLRAEDT AVYYCARSTA NTRSTYYLNL WGQGTLVTVS SGGGGSGGGG |
| | CD28_CD40 | SGGGGSDIQM TQSPSSLSAS VGDRVTITCQ ASQRISSYLS WYQQKPGKVP |
| | | KLLIYGASTL ASGVPSRFSG SGSGTDFTLT ISSLQPEDVA TYYCQSYAYF |
| | | DSNNWHAFGG GTKVEIAAAG SGGSGFVPVF LPAKPTTTPA PRPPTPAPTI |
| | | ASQPLSLRPE AGRPAAGGAV HTRGLDFACD IYIWAPLAGT CGVLLLSLVI |
| | | TLYCNHRNRS KRSRLLHSDY MNMTPRRPGP TRKHYQPYAP PRDFAAYRSK |
| | | KVAKKPTNKA PHPKQEPQEI NFPDDLPGSN TAAPVQETLH GCQPVTQEDG |
| | | KESRISVQER Q |
| 275 | hIgGk-VIII_ | MEAPAQLLFL LLLWLPDTTR EVQLVESGGG LVQPGGSLRL SCAASGFDLG |
| | HuYP218_ | FYFYACWVRQ APGKGLEWVS GIYTAGSGST YYASWAKGRF TISRDNSKNT |
| | spCD8_ | LYLQMNSLRA EDTAVYYCAR STANTRSTYY LNLWGQGTLV TVSSGGGGSG |
| | CD28_CD40 | GGGSGGGGSD IQMTQSPSSL SASVGDRVTI TCQASQRISS YLSWYQQKPG |
| | | KVPKLLIYGA STLASGVPSR FSGSGSGTDF TLTISSLQPE DVATYYCQSY |
| | | AYFDSNNWHA FGGGTKVEIA AAGSGGSGFV PVFLPAKPTT TPAPRPPTPA |
| | | PTIASQPLSL RPEACRPAAG GAVHTRGLDF ACDIYIWAPL AGTCGVLLLS |
| | | LVITLYCNHR NRSKRSRLLH SDYMNMTPRR PGPTRKHYQP YAPPRDFAAY |
| | | RSKKVAKKPT NKAPHPKQEP QEINFPDDLP GSNTAAPVQE TLHGCQPVTQ |
| | | EDGKESRISV QERQ |
| 276 | OSM_HuYP218_ | MGVLLTQRTL LSLVLALLFP SMASMEVQLV ESGGGLVQPG GSLRLSCAAS |
| | CD28TM_ | GFDLGFYFYA CWVRQAPGKG LEWVSCIYTA GSGSTYYASW AKGRFTISRD |
| | CD28_CD40 | NSKNTLYLQM NSLRAEDTAV YYCARSTANT RSTYYLNLWG QGTLVTVSSG |
| | | GGGSGGGGSG GGGSDIQMTQ SPSSLSASVG DRVTITCQAS QRISSYLSWY |
| | | QQKPGKVPKL LIYGASTLAS GVPSRFSGSG SGTDFTLTIS SLQPEDVATY |
| | | YCQSYAYFDS NNWHAFGGGT KVEIAAAGSG GSGFWVLVVV GGVLACYSLL |
| | | VTVAFIIFWV RSKRSRLLHS DYMNMTPRRP GPTRKHYQPY APPRDFAAYR |
| | | SKKVAKKPTN KAPHPKQEPQ EINFPDDLPG SNTAAPVQET LHGCQPVTQE |
| | | DGKESRISVQ ERQ |
| 277 | CD8a_HuYP218_ | MALPVTALLL PLALLLHAAR PEVQLVESGG GLVQPGGSLR LSCAASGFDL |
| | CD28TM_ | GFYFYACWVR QAPGKGLEWV SCIYTAGSGS TYYASWAKGR FTISRDNSKN |
| | CD28_CD40 | TLYLQMNSLR AEDTAVYYCA RSTANTRSTY YLNLWGQGTL VTVSSGGGGS |
| | | GGGGSGGGGS DIQMTQSPSS LSASVGDRVT ITCQASQRIS SYLSWYQQKP |
| | | GKVPKLLIYG ASTLASGVPS RFSGSGSGTD FTLTISSLQP EDVATYYCQS |
| | | YAYFDSNNWH AFGGGTKVEI AAAGSGGSGF WVLVVVGGVL ACYSLLVTVA |
| | | FIIFWVRSKR SRLLHSDYMN MTPRRPGPTR KHYQPYAPPR DFAAYRSKKV |
| | | AKKPTNKAPH PKQEPQEINF PDDLPGSNTA APVQETLHGC QPVTQEDGKE |
| | | SRISVQERQ |
| 278 | CD2_HuYP218_ | MSFPCKFVAS FLLIFNVSSK GAVSEVQLVE SGGGLVQPGG SLRLSCAASG |
| | CD28TM_ | FDLGFYFYAC WVRQAPGKGL EWVSCIYTAG SGSTYYASWA KGRFTISRDN |
| | CD28_CD40 | SKNTLYLQMN SLRAEDTAVY YCARSTANTR STYYLNLWGQ GTLVTVSSGG |
| | | GGSGGGGSGG GGSDIQMTQS PSSLSASVGD RVTITCQASQ RISSYLSWYQ |
| | | QKPGKVPKLL TYGASTLASG VPSRFSGSGS GTDFTLTISS LQPEDVATYY |
| | | CQSYAYFDSN NWHAFGGGTK VEIAAAGSGG SGFWVLVVVG GVLACYSLLV |
| | | TVAFIIFWVR SKRSRLLHSD YMNMTPRRPG PTRKHYQPYA PPRDFAAYRS |
| | | KKVAKKPTNK APHPKQEPQE INFPDDLPGS NTAAPVQETL HGCQPVTQED |
| | | GKESRISVQE RQ |
| 279 | IL2_HuYP218_ | MYRMQLLSCI ALSLALVTNS EVQLVESGGG LVQPGGSLRL SCAASGFDLG |
| | CD28TM_ | FYFYACWVRQ APGKGLEWVS GIYTAGSGST YYASWAKGRF TISRDNSKNT |
| | CD28_CD40 | LYLQMNSLRA EDTAVYYCAR STANTRSTYY LNLWGQGTLV TVSSGGGGSG |
| | | GGGSGGGGSD IQMTQSPSSL SASVGDRVTI TCQASQRISS YLSWYQQKPG |
| | | KVPKLLIYGA STLASGVPSR FSGSGSGTDF TLTISSLQPE DVATYYCQSY |
| | | AYFDSNNWHA FGGGTKVEIA AAGSGGSGFW VLVVVGGVLA CYSLLVTVAF |
| | | IIFWVRSKRS RLLHSDYMNM TPRRPGPTRK HYQPYAPPRD FAAYRSKKVA |
| | | KKPTNKAPHP KQEPQEINFP DDLPGSNTAA PVQETLHGCQ PVTQEDGKES |
| | | RISVQERQ |
| 280 | GM-CSF_HuYP218_ | MWLQSLLLLG TVACSISEVQ LVESGGGLVQ PGGSLRLSCA ASGFDLGFYF |
| | CD28TM_ | YACWVRQAPG KGLEWVSCIY TAGSGSTYYA SWAKGRFTIS RDNSKNTLYL |
| | CD28_CD40 | QMNSLRAEDT AVYYCARSTA NTRSTYYLNL WGQGTLVTVS SGGGGSGGGG |
| | | SGGGGSDIQM TQSPSSLSAS VGDRVTITCQ ASQRISSYLS WYQQKPGKVP |
| | | KLLIYGASTL ASGVPSRFSG SGSGTDFTLT ISSLQPEDVA TYYCQSYAYF |
| | | DSNNWHAFGG GTKVEIAAAG SGGSGFWVLV VVGGVLACYS LLVTVAFIIF |
| | | WVRSKRSRLL HSDYMNMTPR RPGPTRKHYQ PYAPPRDFAA YRSKKVAKKP |
| | | TNKAPHPKQE PQEINFPDDL PGSNTAAPVQ ETLHGCQPVT QEDGKESRIS |
| | | VQERQ |
| 281 | hIgGk-VIII_ | MEAPAQLLFL LLLWLPDTTR EVQLVESGGG LVQPGGSLRL SCAASGFDLG |
| | HuYP218_ | FYFYACWVRQ APGKGLEWVS GIYTAGSGST YYASWAKGRF TISRDNSKNT |
| | CD28TM_ | LYLQMNSLRA EDTAVYYCAR STANTRSTYY LNLWGQGTLV TVSSGGGGSG |
| | CD28_CD40 | GGGSGGGGSD IQMTQSPSSL SASVGDRVTI TCQASQRISS YLSWYQQKPG |
| | | KVPKLLIYGA STLASGVPSR FSGSGSGTDF TLTISSLQPE DVATYYCQSY |
| | | AYFDSNNWHA FGGGTKVEIA AAGSGGSGFW VLVVVGGVLA CYSLLVTVAF |
| | | IIFWVRSKRS RLLHSDYMNM TPRRPGPTRK HYQPYAPPRD FAAYRSKKVA |
| | | KKPTNKAPHP KQEPQEINFP DDLPGSNTAA PVQETLHGCQ PVTQEDGKES |
| | | RISVQERQ |
| 282 | OSM_P4_ | MGVLLTQRTL LSLVLALLFP SMASMQVQLQ QSGPGLVTPS QTLSLTCAIS |
| | spCD28_ | GDSVSSNSAT WNWIRQSPSR GLEWLGRTYY RSKWYNDYAV SVKSRMSINP |
| | CD28_CD40 | DTSKNQFSLQ LNSVTPEDTA VYYCARGMMT YYYGMDVWGQ GTTVTVSSGG |
| | CTP229 | GGSGGGGSGG GGSQPVLTQS SSLSASPGAS ASLTCTLRSG INVGPYRIYW |
| | | YQQKPGSPPQ YLLNYKSDSD KQQGSGVPSR FSGSKDASAN AGVLLISGLR |
| | | SEDEADYYCM IWHSSAAVFG GGTQLTVLSA AAGSGGSGIL VKQSPMLVAY |
| | | DNAVNLSCKY SYNLFSREFR ASLHKGLDSA VEVCVVYGNY SQQLQVYSKT |
| | | GFNCDGKLGN ESVTFYLQNL YVNQTDIYFC KIEVMYPPPY LDNEKSNGTI |
| | | IHVKGKHLCP SPLFPGPSKP FWVLVVVGGV LACYSLLVTV AFIIFWVRSK |
| | | RSRLLHSDYM NMTPRRPGPT RKHYQPYAPP RDFAAYRSKK VAKKPTNKAP |
| | | HPKQEPQEIN FPDDLPGSNT AAPVQETLHG CQPVTQEDGK ESRISVQERQ |
| 283 | CD8a_P4_ | MALPVTALLL PLALLLHAAR PQVQLQQSGP GLVTPSQTLS LTCAISGDSV |
| | spCD28_ | SSNSATWNWI RQSPSRGLEW LGRTYYRSKW YNDYAVSVKS RMSINPDTSK |
| | CD28_CD40 | NQFSLQLNSV TPEDTAVYYC ARGMMTYYYG MDVWGQGTTV TVSSGGGGSG |
| | | GGGSGGGGSQ PVLTQSSSLS ASPGASASLT CTLRSGINVG PYRIYWYQQK |
| | | PGSPPQYLLN YKSDSDKQQG SGVPSRFSGS KDASANAGVL LISGLRSEDE |
| | | ADYYCMIWHS SAAVFGGGTQ LTVLSAAAGS GGSGILVKQS PMLVAYDNAV |
| | | NLSCKYSYNL FSREFRASLH KGLDSAVEVC VVYGNYSQQL QVYSKTGFNC |
| | | DGKLGNESVT FYLQNLYVNQ TDIYFCKIEV MYPPPYLDNE KSNGTIIHVK |
| | | GKHLCPSPLF PGPSKPFWVL VVVGGVLACY SLLVTVAFII FWVRSKRSRL |
| | | LHSDYMNMTP RRPGPTRKHY QPYAPPRDFA AYRSKKVAKK PTNKAPHPKQ |
| | | EPQEINFPDD LPGSNTAAPV QETLHGCQPV TQEDGKESRI SVQERQ |
| 284 | CD2_P4_ | MSFPCKFVAS FLLIFNVSSK GAVSQVQLQQ SGPGLVTPSQ TLSLTCAISG |
| | spCD28_ | DSVSSNSATW NWIRQSPSRG LEWLGRTYYR SKWYNDYAVS VKSRMSINPD |
| | CD28_CD40 | TSKNQFSLQL NSVTPEDTAV YYCARGMMTY YYGMDVWGQG TTVTVSSGGG |
| | | GSGGGGSGGG GSQPVLTQSS SLSASPGASA SLTCTLRSGI NVGPYRIYWY |
| | | QQKPGSPPQY LLNYKSDSDK QQGSGVPSRF SGSKDASANA GVLLISGLRS |
| | | EDEADYYCMI WHSSAAVFGG GTQLTVLSAA AGSGGSGILV KQSPMLVAYD |
| | | NAVNLSCKYS YNLFSREFRA SLHKGLDSAV EVCVVYGNYS QQLQVYSKTG |
| | | FNCDGKLGNE SVTFYLQNLY VNQTDIYFCK IEVMYPPPYL DNEKSNGTII |
| | | HVKGKHLCPS PLFPGPSKPF WVLVVVGGVL ACYSLLVTVA FIIFWVRSKR |
| | | SRLLHSDYMN MTPRRPGPTR KHYQPYAPPR DFAAYRSKKV AKKPTNKAPH |
| | | PKQEPQEINF PDDLPGSNTA APVQETLHGC QPVTQEDGKE SRISVQERQ |
| 285 | IL2_P4_ | MYRMQLLSCI ALSLALVTNS QVQLQQSGPG LVTPSQTLSL TCAISGDSVS |
| | spCD28_ | SNSATWNWIR QSPSRGLEWL GRTYYRSKWY NDYAVSVKSR MSINPDTSKN |
| | CD28_CD40 | QFSLQLNSVT PEDTAVYYCA RGMMTYYYGM DVWGQGTTVT VSSGGGGSGG |
| | | GGSGGGGSQP VLTQSSSLSA SPGASASLTC TLRSGINVGP YRIYWYQQKP |
| | | GSPPQYLLNY KSDSDKQQGS GVPSRFSGSK DASANAGVLL ISGLRSEDEA |
| | | DYYCMIWHSS AAVFGGGTQL TVLSAAAGSG GSGILVKQSP MLVAYDNAVN |
| | | LSCKYSYNLF SREFRASLHK GLDSAVEVCV VYGNYSQQLQ VYSKTGFNCD |
| | | GKLGNESVTF YLQNLYVNQT DIYFCKIEVM YPPPYLDNEK SNGTIIHVKG |
| | | KHLCPSPLFP GPSKPFWVLV VVGGVLACYS LLVTVAFIIF WVRSKRSRLL |
| | | HSDYMNMTPR RPGPTRKHYQ PYAPPRDFAA YRSKKVAKKP TNKAPHPKQE |
| | | PQEINFPDDL PGSNTAAPVQ ETLHGCQPVT QEDGKESRIS VQERQ |
| 286 | GM-CSF_P4_ | MWLQSLLLLG TVACSISQVQ LQQSGPGLVT PSQTLSLTCA ISGDSVSSNS |
| | spCD28_ | ATWNWIRQSP SRGLEWLGRT YYRSKWYNDY AVSVKSRMSI NPDTSKNQFS |
| | CD28_CD40 | LQLNSVTPED TAVYYCARGM MTYYYGMDVW GQGTTVTVSS GGGGSGGGGS |
| | | GGGGSQPVLT QSSSLSASPG ASASLTCTLR SGINVGPYRI YWYQQKPGSP |
| | | PQYLLNYKSD SDKQQGSGVP SRFSGSKDAS ANAGVLLISG LRSEDEADYY |
| | | CMIWHSSAAV FGGGTQLTVL SAAAGSGGSG ILVKQSPMLV AYDNAVNLSC |
| | | KYSYNLFSRE FRASLHKGLD SAVEVCVVYG NYSQQLQVYS KTGFNCDGKL |
| | | GNESVTFYLQ NLYVNQTDIY FCKIEVMYPP PYLDNEKSNG TIIHVKGKHL |
| | | CPSPLFPGPS KPFWVLVVVG GVLACYSLLV TVAFIIFWVR SKRSRLLHSD |
| | | YMNMTPRRPG PTRKHYQPYA PPRDFAAYRS KKVAKKPTNK APHPKQEPQE |
| | | INFPDDLPGS NTAAPVQETL HGCQPVTQED GKESRISVQE RQ |
| 287 | hIgGk-VIII_ | MEAPAQLLFL LLLWLPDTTR QVQLQQSGPG LVTPSQTLSL TCAISGDSVS |
| | P4_ | SNSATWNWIR QSPSRGLEWL GRTYYRSKWY NDYAVSVKSR MSINPDTSKN |
| | spCD28_ | QFSLQLNSVT PEDTAVYYCA RGMMTYYYGM DVWGQGTTVT VSSGGGGSGG |
| | CD28_CD40 | GGSGGGGSQP VLTQSSSLSA SPGASASLTC TLRSGINVGP YRIYWYQQKP |
| | | GSPPQYLLNY KSDSDKQQGS GVPSRFSGSK DASANAGVLL ISGLRSEDEA |
| | | DYYCMIWHSS AAVFGGGTQL TVLSAAAGSG GSGILVKQSP MLVAYDNAVN |
| | | LSCKYSYNLF SREFRASLHK GLDSAVEVCV VYGNYSQQLQ VYSKTGFNCD |
| | | GKLGNESVTF YLQNLYVNQT DIYFCKIEVM YPPPYLDNEK SNGTIIHVKG |
| | | KHLCPSPLFP GPSKPFWVLV VVGGVLACYS LLVTVAFIIF WVRSKRSRLL |
| | | HSDYMNMTPR RPGPTRKHYQ PYAPPRDFAA YRSKKVAKKP TNKAPHPKQE |
| | | PQEINFPDDL PGSNTAAPVQ ETLHGCQPVT QEDGKESRIS VQERQ |
| 288 | OSM_P4_spCD8_ | MGVLLTQRTL LSLVLALLFP SMASMQVOLQ QSGPGLVTPS QTLSLTCAIS |
| | CD28_CD40 | GDSVSSNSAT WNWIRQSPSR GLEWLGRTYY RSKWYNDYAV SVKSRMSINP |
| | CTP241 | DTSKNQFSLQ LNSVTPEDTA VYYCARGMMT YYYGMDVWGQ GTTVTVSSGG |
| | | GGSGGGGSGG GGSQPVLTQS SSLSASPGAS ASLTCTLRSG INVGPYRIYW |
| | | YQQKPGSPPQ YLLNYKSDSD KQQGSGVPSR FSGSKDASAN AGVLLISGLR |
| | | SEDEADYYGM IWHSSAAVFG GGTQLTVLSA AAGSGGSGFV PVFLPAKPTT |
| | | TPAPRPPTPA PTIASQPLSL RPEACRPAAG GAVHTRGLDF ACDIYIWAPL |
| | | AGTCGVLLLS LVITLYCNHR NRSKRSRLLH SDYMNMTPRR PGPTRKHYQP |
| | | YAPPRDFAAY RSKKVAKKPT NKAPHPKQEP QEINFPDDLP GSNTAAPVQE |
| | | TLHGCQPVTQ EDGKESRISV QERQ |
| 289 | CD8a_P4_spCD8_ | MALPVTALLL PLALLLHAAR PQVQLQQSGP GLVTPSQTLS LTCAISGDSV |
| | CD28_CD40 | SSNSATWNWI RQSPSRGLEW LGRTYYRSKW YNDYAVSVKS RMSINPDTSK |
| | | NQFSLQLNSV TPEDTAVYYC ARGMMTYYYG MDVWGQGTTV TVSSGGGGSG |
| | | GGGSGGGGSQ PVLTQSSSLS ASPGASASLT CTLRSGINVG PYRIYWYQQK |
| | | PGSPPQYLLN YKSDSDKQQG SGVPSRFSGS KDASANAGVL LISGLRSEDE |
| | | ADYYCMIWHS SAAVFGGGTQ LTVLSAAAGS GGSGFVPVFL PAKPTTTPAP |
| | | RPPTPAPTIA SQPLSLRPEA CRPAAGGAVH TRGLDFACDI YIWAPLAGTC |
| | | GVLLLSLVIT LYCNHRNRSK RSRLLHSDYM NMTPRRPGPT RKHYQPYAPP |
| | | RDFAAYRSKK VAKKPTNKAP HPKQEPQEIN FPDDLPGSNT AAPVQETLHG |
| | | CQPVTQEDGK ESRISVQERQ |
| 290 | CD2_P4_spCD8_ | MSFPCKFVAS FLLIFNVSSK GAVSQVQLQQ SGPGLVTPSQ TLSLTCAISG |
| | CD28_CD40 | DSVSSNSATW NWIRQSPSRG LEWLGRTYYR SKWYNDYAVS VKSRMSINPD |
| | | TSKNQFSLQL NSVTPEDTAV YYCARGMMTY YYGMDVWGQG TTVTVSSGGG |
| | | GSGGGGSGGG GSQPVLTQSS SLSASPGASA SLTCTLRSGI NVGPYRIYWY |
| | | QQKPGSPPQY LLNYKSDSDK QQGSGVPSRF SGSKDASANA GVLLISGLRS |
| | | EDEADYYCMI WHSSAAVFGG GTQLTVLSAA AGSGGSGFVP VFLPAKPTTT |
| | | PAPRPPTPAP TIASQPLSLR PEACRPAAGG AVHTRGLDFA CDIYIWAPLA |
| | | GTCGVLLLSL VITLYCNHRN RSKRSRLLHS DYMNMTPRRP GPTRKHYQPY |
| | | APPRDFAAYR SKKVAKKPTN KAPHPKQEPQ EINFPDDLPG SNTAAPVQET |
| | | LHGCQPVTQE DGKESRISVQ ERQ |
| 291 | IL2_P4_spCD8_ | MYRMQLLSCI ALSLALVTNS QVQLQQSGPG LVTPSQTLSL TCAISGDSVS |
| | CD28_CD40 | SNSATWNWIR QSPSRGLEWL GRTYYRSKWY NDYAVSVKSR MSINPDTSKN |
| | | QFSLQLNSVT PEDTAVYYCA RGMMTYYYGM DVWGQGTTVT VSSGGGGSGG |
| | | GGSGGGGSQP VLTQSSSLSA SPGASASLTC TLRSGINVGP YRIYWYQQKP |
| | | GSPPQYLLNY KSDSDKQQGS GVPSRFSGSK DASANAGVLL ISGLRSEDEA |
| | | DYYCMIWHSS AAVFGGGTQL TVLSAAAGSG GSGFVPVFLP AKPTTTPAPR |
| | | PPTPAPTIAS QPLSLRPEAC RPAAGGAVHT RGLDFACDIY IWAPLAGTCG |
| | | VLLLSLVITL YCNHRNRSKR SRLLHSDYMN MTPRRPGPTR KHYQPYAPPR |
| | | DFAAYRSKKV AKKPTNKAPH PKQEPQEINF PDDLPGSNTA APVQETLHGC |
| | | QPVTQEDGKE SRISVQERQ |
| 292 | GM-CSF_P4_ | MWLQSLLLLG TVACSISQVQ LQQSGPGLVT PSQTLSLTCA ISGDSVSSNS |
| | spCD8_CD28_CD40 | ATWNWIRQSP SRGLEWLGRT YYRSKWYNDY AVSVKSRMSI NPDTSKNQFS |
| | | LQLNSVTPED TAVYYCARGM MTYYYGMDVW GQGTTVTVSS GGGGSGGGGS |
| | | GGGGSQPVLT QSSSLSASPG ASASLTCTLR SGINVGPYRI YWYQQKPGSP |
| | | PQYLLNYKSD SDKQQGSGVP SRFSGSKDAS ANAGVLLISG LRSEDEADYY |
| | | CMIWHSSAAV FGGGTQLTVL SAAAGSGGSG FVPVFLPAKP TTTPAPRPPT |
| | | PAPTIASQPL SLRPEACRPA AGGAVHTRGL DFACDIYIWA PLAGTCGVLL |
| | | LSLVITLYCN HRNRSKRSRL LHSDYMNMTP RRPGPTRKHY QPYAPPRDFA |
| | | AYRSKKVAKK PTNKAPHPKQ EPQEINFPDD LPGSNTAAPV QETLHGCQPV |
| | | TQEDGKESRI SVQERQ |
| 293 | hIgGk-VIII_ | MEAPAQLLFL LLLWLPDTTR QVQLQQSGPG LVTPSQTLSL TCAISGDSVS |
| | P4_spCD8_ | SNSATWNWIR QSPSRGLEWL GRTYYRSKWY NDYAVSVKSR MSINPDTSKN |
| | CD28_CD40 | QFSLQLNSVT PEDTAVYYCA RGMMTYYYGM DVWGQGTTVT VSSGGGGSGG |
| | | GGSGGGGSQP VLTQSSSLSA SPGASASLTC TLRSGINVGP YRIYWYQQKP |
| | | GSPPQYLLNY KSDSDKQQGS GVPSRFSGSK DASANAGVLL ISGLRSEDEA |
| | | DYYCMIWHSS AAVFGGGTQL TVLSAAAGSG GSGFVPVFLP AKPTTTPAPR |
| | | PPTPAPTIAS QPLSLRPEAC RPAAGGAVHT RGLDFACDIY IWAPLAGTCG |
| | | VLLLSLVITL YCNHRNRSKR SRLLHSDYMN MTPRRPGPTR KHYQPYAPPR |
| | | DFAAYRSKKV AKKPTNKAPH PKQEPQEINF PDDLPGSNTA APVQETLHGC |
| | | QPVTQEDGKE SRISVQERQ |
| 294 | OSM_P4_CD28TM_ | MGVLLTQRTL LSLVLALLFP SMASMQVOLQ QSGPGLVTPS QTLSLTCAIS |
| | CD28_CD40 | GDSVSSNSAT WNWIRQSPSR GLEWLGRTYY RSKWYNDYAV SVKSRMSINP |
| | | DTSKNQFSLQ LNSVTPEDTA VYYCARGMMT YYYGMDVWGQ GTTVTVSSGG |
| | | GGSGGGGSGG GGSQPVLTQS SSLSASPGAS ASLTCTLRSG INVGPYRIYW |
| | | YQQKPGSPPQ YLLNYKSDSD KQQGSGVPSR FSGSKDASAN AGVLLISGLR |
| | | SEDEADYYGM IWHSSAAVFG GGTQLTVLSA AAGSGGSGFW VLVVVGGVLA |
| | | CYSLLVTVAF IIFWVRSKRS RLLHSDYMNM TPRRPGPTRK HYQPYAPPRD |
| | | FAAYRSKKVA KKPTNKAPHP KQEPQEINFP DDLPGSNTAA PVQETLHGCQ |
| | | PVTQEDGKES RISVQERQ |
| 295 | CD8a_P4_ | MALPVTALLL PLALLLHAAR PQVQLQQSGP GLVTPSQTLS LTCAISGDSV |
| | CD28TM_CD28_ | SSNSATWNWI RQSPSRGLEW LGRTYYRSKW YNDYAVSVKS RMSINPDTSK |
| | CD40 | NQFSLQLNSV TPEDTAVYYC ARGMMTYYYG MDVWGQGTTV TVSSGGGGSG |
| | | GGGSGGGGSQ PVLTQSSSLS ASPGASASLT CTLRSGINVG PYRIYWYQQK |
| | | PGSPPQYLLN YKSDSDKQQG SGVPSRFSGS KDASANAGVL LISGLRSEDE |
| | | ADYYCMIWHS SAAVFGGGTQ LTVLSAAAGS GGSGFWVLVV VGGVLACYSL |
| | | LVTVAFIIFW VRSKRSRLLH SDYMNMTPRR PGPTRKHYQP YAPPRDFAAY |
| | | RSKKVAKKPT NKAPHPKQEP QEINFPDDLP GSNTAAPVQE TLHGCQPVTQ |
| | | EDGKESRISV QERQ |
| 296 | CD2_P4_CD28TM_ | MSFPCKFVAS FLLIFNVSSK GAVSQVQLQQ SGPGLVTPSQ TLSLTCAISG |
| | CD28_CD40 | DSVSSNSATW NWIRQSPSRG LEWLGRTYYR SKWYNDYAVS VKSRMSINPD |
| | | TSKNQFSLQL NSVTPEDTAV YYCARGMMTY YYGMDVWGQG TTVTVSSGGG |
| | | GSGGGGSGGG GSQPVLTQSS SLSASPGASA SLTCTLRSGI NVGPYRIYWY |
| | | QQKPGSPPQY LLNYKSDSDK QQGSGVPSRF SGSKDASANA GVLLISGLRS |
| | | EDEADYYCMI WHSSAAVFGG GTQLTVLSAA AGSGGSGFWV LVVVGGVLAC |
| | | YSLLVTVAFI IFWVRSKRSR LLHSDYMNMT PRRPGPTRKH YQPYAPPRDF |
| | | AAYRSKKVAK KPTNKAPHPK QEPQEINFPD DLPGSNTAAP VQETLHGCQP |
| | | VTQEDGKESR ISVQERQ |
| 297 | IL2_P4_CD28TM_ | MYRMQLLSCI ALSLALVTNS QVQLQQSGPG LVTPSQTLSL TCAISGDSVS |
| | CD28_CD40 | SNSATWNWIR QSPSRGLEWL GRTYYRSKWY NDYAVSVKSR MSINPDTSKN |
| | | QFSLQLNSVT PEDTAVYYCA RGMMTYYYGM DVWGQGTTVT VSSGGGGSGG |
| | | GGSGGGGSQP VLTQSSSLSA SPGASASLTC TLRSGINVGP YRIYWYQQKP |
| | | GSPPQYLLNY KSDSDKQQGS GVPSRFSGSK DASANAGVLL ISGLRSEDEA |
| | | DYYCMIWHSS AAVFGGGTQL TVLSAAAGSG GSGFWVLVVV GGVLACYSLL |
| | | VTVAFIIFWV RSKRSRLLHS DYMNMTPRRP GPTRKHYQPY APPRDFAAYR |
| | | SKKVAKKPTN KAPHPKQEPQ EINFPDDLPG SNTAAPVQET LHGCQPVTQE |
| | | DGKESRISVQ ERQ |
| 298 | GM-CSF_P4_ | MWLQSLLLLG TVACSISQVQ LQQSGPGLVT PSQTLSLTCA ISGDSVSSNS |
| | CD28TM_ | ATWNWIRQSP SRGLEWLGRT YYRSKWYNDY AVSVKSRMSI NPDTSKNQFS |
| | CD28_CD40 | LQLNSVTPED TAVYYCARGM MTYYYGMDVW GQGTTVTVSS GGGGSGGGGS |
| | | GGGGSQPVLT QSSSLSASPG ASASLTCTLR SGINVGPYRI YWYQQKPGSP |
| | | PQYLLNYKSD SDKQQGSGVP SRFSGSKDAS ANAGVLLISG LRSEDEADYY |
| | | CMIWHSSAAV FGGGTQLTVL SAAAGSGGSG FWVLVVVGGV LACYSLLVTV |
| | | AFIIFWVRSK RSRLLHSDYM NMTPRRPGPT RKHYQPYAPP RDFAAYRSKK |
| | | VAKKPTNKAP HPKQEPQEIN FPDDLPGSNT AAPVQETLHG CQPVTQEDGK |
| | | ESRISVQERQ |
| 299 | hIgGk-VIII_ | MEAPAQLLFL LLLWLPDTTR QVQLQQSGPG LVTPSQTLSL TCAISGDSVS |
| | P4_CD28TM_ | SNSATWNWIR QSPSRGLEWL GRTYYRSKWY NDYAVSVKSR MSINPDTSKN |
| | CD28_CD40 | QFSLQLNSVT PEDTAVYYCA RGMMTYYYGM DVWGQGTTVT VSSGGGGSGG |
| | | GGSGGGGSQP VLTQSSSLSA SPGASASLTC TLRSGINVGP YRIYWYQQKP |
| | | GSPPQYLLNY KSDSDKQQGS GVPSRFSGSK DASANAGVLL ISGLRSEDEA |
| | | DYYCMIWHSS AAVFGGGTQL TVLSAAAGSG GSGFWVLVVV GGVLACYSLL |
| | | VTVAFIIFWV RSKRSRLLHS DYMNMTPRRP GPTRKHYQPY APPRDFAAYR |
| | | SKKVAKKPTN KAPHPKQEPQ EINFPDDLPG SNTAAPVQET LHGCQPVTQE |
| | | DGKESRISVQ ERQ |
| 300 | OSM_SS1_ | MGVLLTQRTL LSLVLALLFP SMASMQVQLQ QSGPELEKPG ASVKLSCKAS |
| | spCD28(trun)_ | GYSFTGYTMN WVKQSHGKSL EWIGLITPYN GASSYNQKFR GKATLTVDKS |
| | CD28_CD40 | SSTAYMDLLS LTSEDSAVYF CARGGYDGRG FDYWGQGTTV TVSSGGGGSG |
| | CTP248 | GGGSGGGGSD IELTQSPAIM SASPGEKVTM TCSASSSVSY MHWYQQKSGT |
| | | SPKRWIYDTS KLASGVPGRF SGSGSGNSYS LTISSVEAED DATYYCQQWS |
| | | KHPLTFGAGT KLEIKAAAGS GGSGIIHVKG KHLCPSPLFP GPSKPFWVLV |
| | | VVGGVLACYS LLVTVAFIIF WVRSKRSRLL HSDYMNMTPR RPGPTRKHYQ |
| | | PYAPPRDFAA YRSKKVAKKP TNKAPHPKQE PQEINFPDDL PGSNTAAPVQ |
| | | ETLHGCQPVT QEDGKESRIS VQERQ |
| 301 | CD8a_SS1_ | MALPVTALLL PLALLLHAAR PQVQLQQSGP ELEKPGASVK LSCKASGYSF |
| | spCD28(trun)_ | TGYTMNWVKQ SHGKSLEWIG LITPYNGASS YNQKFRGKAT LTVDKSSSTA |
| | CD28_CD40 | YMDLLSLTSE DSAVYFCARG GYDGRGFDYW GQGTTVTVSS GGGGSGGGGS |
| | | GGGGSDIELT QSPAIMSASP GEKVTMTCSA SSSVSYMHWY QQKSGTSPKR |
| | | WIYDTSKLAS GVPGRFSGSG SGNSYSLTIS SVEAEDDATY YCQQWSKHPL |
| | | TFGAGTKLEI KAAAGSGGSG IIHVKGKHLC PSPLFPGPSK PFWVLVVVGG |
| | | VLACYSLLVT VAFIIFWVRS KRSRLLHSDY MNMTPRRPGP TRKHYQPYAP |
| | | PRDFAAYRSK KVAKKPTNKA PHPKQEPQEI NFPDDLPGSN TAAPVQETLH |
| | | GCQPVTQEDG KESRISVQER Q |
| 302 | CD2_SS1_ | MSFPCKFVAS FLLIFNVSSK GAVSQVQLQQ SGPELEKPGA SVKLSCKASG |
| | spCD28(trun)_ | YSFTGYTMNW VKQSHGKSLE WIGLITPYNG ASSYNQKFRG KATLTVDKSS |
| | CD28_CD40 | STAYMDLLSL TSEDSAVYFC ARGGYDGRGF DYWGQGTTVT VSSGGGGSGG |
| | | GGSGGGGSDI ELTQSPAIMS ASPGEKVTMT CSASSSVSYM HWYQQKSGTS |
| | | PKRWIYDTSK LASGVPGRFS GSGSGNSYSL TISSVEAEDD ATYYCQQWSK |
| | | HPLTFGAGTK LEIKAAAGSG GSGIIHVKGK HLCPSPLFPG PSKPFWVLVV |
| | | VGGVLACYSL LVTVAFIIFW VRSKRSRLLH SDYMNMTPRR PGPTRKHYQP |
| | | YAPPRDFAAY RSKKVAKKPT NKAPHPKQEP QEINFPDDLP GSNTAAPVQE |
| | | TLHGCQPVTQ EDGKESRISV QERQ |
| 303 | IL2_SS1_ | MYRMQLLSCI ALSLALVTNS QVQLQQSGPE LEKPGASVKL SCKASGYSFT |
| | spCD28(trun)_ | GYTMNWVKQS HGKSLEWIGL ITPYNGASSY NQKFRGKATL TVDKSSSTAY |
| | CD28_CD40 | MDLLSLTSED SAVYFCARGG YDGRGFDYWG QGTTVTVSSG GGGSGGGGSG |
| | | GGGSDIELTQ SPAIMSASPG EKVTMTCSAS SSVSYMHWYQ QKSGTSPKRW |
| | | IYDTSKLASG VPGRFSGSGS GNSYSLTISS VEAEDDATYY CQQWSKHPLT |
| | | FGAGTKLEIK AAAGSGGSGI IHVKGKHLCP SPLFPGPSKP FWVLVVVGGV |
| | | LACYSLLVTV AFIIFWVRSK RSRLLHSDYM NMTPRRPGPT RKHYQPYAPP |
| | | RDFAAYRSKK VAKKPTNKAP HPKQEPQEIN FPDDLPGSNT AAPVQETLHG |
| | | CQPVTQEDGK ESRISVQERQ |
| 304 | GM-CSF_SS1_ | MWLQSLLLLG TVACSISQVQ LQQSGPELEK PGASVKLSCK ASGYSFTGYT |
| | spCD28(trun)_ | MNWVKQSHGK SLEWIGLITP YNGASSYNQK FRGKATLTVD KSSSTAYMDL |
| | CD28_CD40 | LSLTSEDSAV YFCARGGYDG RGFDYWGQGT TVTVSSGGGG SGGGGSGGGG |
| | | SDIELTQSPA IMSASPGEKV TMTCSASSSV SYMHWYQQKS GTSPKRWIYD |
| | | TSKLASGVPG RFSGSGSGNS YSLTISSVEA EDDATYYCQQ WSKHPLTFGA |
| | | GTKLEIKAAA GSGGSGIIHV KGKHLCPSPL FPGPSKPFWV LVVVGGVLAC |
| | | YSLLVTVAFI IFWVRSKRSR LLHSDYMNMT PRRPGPTRKH YQPYAPPRDF |
| | | AAYRSKKVAK KPTNKAPHPK QEPQEINFPD DLPGSNTAAP VQETLHGCQP |
| | | VTQEDGKESR ISVQERQ |
| 305 | hIgGk-VIII_ | MEAPAQLLFL LLLWLPDTTR QVQLQQSGPE LEKPGASVKL SCKASGYSFT |
| | SS1_ | GYTMNWVKQS HGKSLEWIGL ITPYNGASSY NQKFRGKATL TVDKSSSTAY |
| | spCD28(trun)_ | MDLLSLTSED SAVYFCARGG YDGRGFDYWG QGTTVTVSSG GGGSGGGGSG |
| | CD28_CD40 | GGGSDIELTQ SPAIMSASPG EKVTMTCSAS SSVSYMHWYQ QKSGTSPKRW |
| | | IYDTSKLASG VPGRFSGSGS GNSYSLTISS VEAEDDATYY CQQWSKHPLT |
| | | FGAGTKLEIK AAAGSGGSGI IHVKGKHLCP SPLFPGPSKP FWVLVVVGGV |
| | | LACYSLLVTV AFIIFWVRSK RSRLLHSDYM NMTPRRPGPT RKHYQPYAPP |
| | | RDFAAYRSKK VAKKPTNKAP HPKQEPQEIN FPDDLPGSNT AAPVQETLHG |
| | | CQPVTQEDGK ESRISVQERQ |
| 306 | OSM_M5_ | MGVLLTQRTL LSLVLALLFP SMASMQVQLV QSGAEVEKPG ASVKVSCKAS |
| | spCD28(trun)_ | GYTFTDYYMH WVRQAPGQGL EWMGWINPNS GGTNYAQKFQ GRVTMTRDTS |
| | CD28_CD40 | ISTAYMELSR LRSDDTAVYY CASGWDFDYW GQGTLVTVSS GGGGSGGGGS |
| | CTP249 | GGGGSDIVMT QSPSSLSASV GDRVTITCRA SQSIRYYLSW YQQKPGKAPK |
| | | LLIYTASILQ NGVPSRFSGS GSGTDFTLTI SSLQPEDFAT YYCLQTYTTP |
| | | DFGPGTKVEI KAAAGSGGSG IIHVKGKHLC PSPLFPGPSK PFWVLVVVGG |
| | | VLACYSLLVT VAFIIFWVRS KRSRLLHSDY MNMTPRRPGP TRKHYQPYAP |
| | | PRDFAAYRSK KVAKKPTNKA PHPKQEPQEI NFPDDLPGSN TAAPVQETLH |
| | | GCQPVTQEDG KESRISVQER Q |
| 307 | CD8a_M5_ | MALPVTALLL PLALLLHAAR PQVQLVQSGA EVEKPGASVK VSCKASGYTF |
| | spCD28(trun)_ | TDYYMHWVRQ APGQGLEWMG WINPNSGGTN YAQKFQGRVT MTRDTSISTA |
| | CD28_CD40 | YMELSRLRSD DTAVYYCASG WDFDYWGQGT LVTVSSGGGG SGGGGSGGGG |
| | | SDIVMTQSPS SLSASVGDRV TITCRASQSI RYYLSWYQQK PGKAPKLLIY |
| | | TASILQNGVP SRFSGSGSGT DFTLTISSLQ PEDFATYYCL QTYTTPDFGP |
| | | GTKVEIKAAA GSGGSGIIHV KGKHLCPSPL FPGPSKPFWV LVVVGGVLAC |
| | | YSLLVTVAFI IFWVRSKRSR LLHSDYMNMT PRRPGPTRKH YQPYAPPRDF |
| | | AAYRSKKVAK KPTNKAPHPK QEPQEINFPD DLPGSNTAAP VQETLHGCQP |
| | | VTQEDGKESR ISVQERQ |
| 308 | CD2_M5_ | MSFPCKFVAS FLLIFNVSSK GAVSQVQLVQ SGAEVEKPGA SVKVSCKASG |
| | spCD28(trun)_ | YTFTDYYMHW VRQAPGQGLE WMGWINPNSG GTNYAQKFQG RVTMTRDTSI |
| | CD28_CD40 | STAYMELSRL RSDDTAVYYC ASGWDFDYWG QGTLVTVSSG GGGSGGGGSG |
| | | GGGSDIVMTQ SPSSLSASVG DRVTITCRAS QSIRYYLSWY QQKPGKAPKL |
| | | LIYTASILON GVPSRFSGSG SGTDFTLTIS SLQPEDFATY YCLQTYTTPD |
| | | FGPGTKVEIK AAAGSGGSGI IHVKGKHLCP SPLFPGPSKP FWVLVVVGGV |
| | | LACYSLLVTV AFIIFWVRSK RSRLLHSDYM NMTPRRPGPT RKHYQPYAPP |
| | | RDFAAYRSKK VAKKPTNKAP HPKQEPQEIN FPDDLPGSNT AAPVQETLHG |
| | | CQPVTQEDGK ESRISVQERQ |
| 309 | IL2_M5_ | MYRMQLLSCI ALSLALVTNS QVQLVQSGAE VEKPGASVKV SCKASGYTFT |
| | spCD28(trun)_ | DYYMHWVRQA PGQGLEWMGW INPNSGGTNY AQKFQGRVTM TRDTSISTAY |
| | CD28_CD40 | MELSRLRSDD TAVYYCASGW DFDYWGQGTL VTVSSGGGGS GGGGSGGGGS |
| | | DIVMTQSPSS LSASVGDRVT ITCRASQSIR YYLSWYQQKP GKAPKLLIYT |
| | | ASILQNGVPS RFSGSGSGTD FTLTISSLQP EDFATYYCLQ TYTTPDFGPG |
| | | TKVEIKAAAG SGGSGIIHVK GKHLCPSPLF PGPSKPFWVL VVVGGVLACY |
| | | SLLVTVAFII FWVRSKRSRL LHSDYMNMTP RRPGPTRKHY QPYAPPRDFA |
| | | AYRSKKVAKK PTNKAPHPKQ EPQEINFPDD LPGSNTAAPV QETLHGCQPV |
| | | TQEDGKESRI SVQERQ |
| 310 | GM-CSF_M5_ | MWLQSLLLLG TVACSISQVQ LVQSGAEVEK PGASVKVSCK ASGYTFTDYY |
| | spCD28(trun)_ | MHWVRQAPGQ GLEWMGWINP NSGGTNYAQK FQGRVTMTRD TSISTAYMEL |
| | CD28_CD40 | SRLRSDDTAV YYCASGWDFD YWGQGTLVTV SSGGGGSGGG GSGGGGSDIV |
| | | MTQSPSSLSA SVGDRVTITC RASQSIRYYL SWYQQKPGKA PKLLIYTASI |
| | | LQNGVPSRFS GSGSGTDFTL TISSLQPEDF ATYYCLQTYT TPDFGPGTKV |
| | | EIKAAAGSGG SGIIHVKGKH LCPSPLFPGP SKPFWVLVVV GGVLACYSLL |
| | | VTVAFIIFWV RSKRSRLLHS DYMNMTPRRP GPTRKHYQPY APPRDFAAYR |
| | | SKKVAKKPTN KAPHPKQEPQ EINFPDDLPG SNTAAPVQET LHGCQPVTQE |
| | | DGKESRISVQ ERQ |
| 311 | hIgGk-VIII_ | MEAPAQLLFL LLLWLPDTTR QVQLVQSGAE VEKPGASVKV SCKASGYTFT |
| | M5_ | DYYMHWVRQA PGQGLEWMGW INPNSGGTNY AQKFQGRVTM TRDTSISTAY |
| | spCD28(trun)_ | MELSRLRSDD TAVYYCASGW DFDYWGQGTL VTVSSGGGGS GGGGSGGGGS |
| | CD28_CD40 | DIVMTQSPSS LSASVGDRVT ITCRASQSIR YYLSWYQQKP GKAPKLLIYT |
| | | ASILQNGVPS RFSGSGSGTD FTLTISSLQP EDFATYYCLQ TYTTPDFGPG |
| | | TKVEIKAAAG SGGSGIIHVK GKHLCPSPLF PGPSKPFWVL VVVGGVLACY |
| | | SLLVTVAFII FWVRSKRSRL LHSDYMNMTP RRPGPTRKHY QPYAPPRDFA |
| | | AYRSKKVAKK PTNKAPHPKQ EPQEINFPDD LPGSNTAAPV QETLHGCQPV |
| | | TQEDGKESRI SVQERQ |
| 312 | OSM_HN1_ | MGVLLTQRTL LSLVLALLFP SMASMQVQLV QSGAEVKRPG ASVQVSCRAS |
| | spCD28(trun)_ | GYSINTYYMQ WVRQAPGAGL EWMGVINPSG VTSYAQKFQG RVTLTNDTST |
| | CD28_CD40 | NTVYMQLNSL TSADTAVYYC ARWALWGDFG MDVWGKGTLV TVSSGGGGSG |
| | CTP250 | GGGSGGGGSD IQMTQSPSTL SASIGDRVTI TGRASEGIYH WLAWYQQKPG |
| | | KAPKLLIYKA SSLASGAPSR FSGSGSGTDF TLTISSLQPD DFATYYCQQY |
| | | SNYPLTFGGG TKLEIKAAAG SGGSGIIHVK GKHLCPSPLF PGPSKPFWVL |
| | | VVVGGVLACY SLLVTVAFII FWVRSKRSRL LHSDYMNMTP RRPGPTRKHY |
| | | QPYAPPRDFA AYRSKKVAKK PTNKAPHPKQ EPQEINFPDD LPGSNTAAPV |
| | | QETLHGCQPV TQEDGKESRI SVQERQ |
| 313 | CD8a_HN1_ | MALPVTALLL PLALLLHAAR PQVQLVQSGA EVKRPGASVQ VSCRASGYSI |
| | spCD28(trun)_ | NTYYMQWVRQ APGAGLEWMG VINPSGVTSY AQKFQGRVTL TNDTSTNTVY |
| | CD28_CD40 | MQLNSLTSAD TAVYYCARWA LWGDFGMDVW GKGTLVTVSS GGGGSGGGGS |
| | | GGGGSDIQMT QSPSTLSASI GDRVTITCRA SEGIYHWLAW YQQKPGKAPK |
| | | LLIYKASSLA SGAPSRFSGS GSGTDFTLTI SSLQPDDFAT YYCQQYSNYP |
| | | LTFGGGTKLE IKAAAGSGGS GIIHVKGKHL CPSPLFPGPS KPFWVLVVVG |
| | | GVLACYSLLV TVAFIIFWVR SKRSRLLHSD YMNMTPRRPG PTRKHYQPYA |
| | | PPRDFAAYRS KKVAKKPTNK APHPKQEPQE INFPDDLPGS NTAAPVQETL |
| | | HGCQPVTQED GKESRISVQE RQ |
| 314 | CD2_HN1_ | MSFPCKFVAS FLLIFNVSSK GAVSQVQLVQ SGAEVKRPGA SVQVSCRASG |
| | spCD28(trun)_ | YSINTYYMOW VRQAPGAGLE WMGVINPSGV TSYAQKFQGR VTLTNDTSTN |
| | CD28_CD40 | TVYMQLNSLT SADTAVYYGA RWALWGDFGM DVWGKGTLVT VSSGGGGSGG |
| | | GGSGGGGSDI QMTQSPSTLS ASIGDRVTIT CRASEGIYHW LAWYQQKPGK |
| | | APKLLIYKAS SLASGAPSRF SGSGSGTDFT LTISSLQPDD FATYYCQQYS |
| | | NYPLTFGGGT KLEIKAAAGS GGSGIIHVKG KHLCPSPLFP GPSKPFWVLV |
| | | VVGGVLACYS LLVTVAFIIF WVRSKRSRLL HSDYMNMTPR RPGPTRKHYQ |
| | | PYAPPRDFAA YRSKKVAKKP TNKAPHPKQE PQEINFPDDL PGSNTAAPVQ |
| | | ETLHGCQPVT QEDGKESRIS VQERQ |
| 315 | IL2_HN1_ | MYRMQLLSCI ALSLALVTNS QVQLVQSGAE VKRPGASVQV SCRASGYSIN |
| | spCD28(trun)_ | TYYMQWVRQA PGAGLEWMGV INPSGVTSYA QKFQGRVTLT NDTSTNTVYM |
| | CD28_CD40 | QLNSLTSADT AVYYCARWAL WGDFGMDVWG KGTLVTVSSG GGGSGGGGSG |
| | | GGGSDIQMTQ SPSTLSASIG DRVTITCRAS EGIYHWLAWY QQKPGKAPKL |
| | | LIYKASSLAS GAPSRFSGSG SGTDFTLTIS SLQPDDFATY YCQQYSNYPL |
| | | TFGGGTKLEI KAAAGSGGSG IIHVKGKHLC PSPLFPGPSK PFWVLVVVGG |
| | | VLACYSLLVT VAFIIFWVRS KRSRLLHSDY MNMTPRRPGP TRKHYQPYAP |
| | | PRDFAAYRSK KVAKKPTNKA PHPKQEPQEI NFPDDLPGSN TAAPVQETLH |
| | | GCQPVTQEDG KESRISVQER Q |
| 316 | GM-CSF_HN1_ | MWLQSLLLLG TVACSISQVQ LVQSGAEVKR PGASVQVSCR ASGYSINTYY |
| | spCD28(trun)_ | MQWVRQAPGA GLEWMGVINP SGVTSYAQKF QGRVTLTNDT STNTVYMQLN |
| | CD28_CD40 | SLTSADTAVY YCARWALWGD FGMDVWGKGT LVTVSSGGGG SGGGGSGGGG |
| | | SDIQMTQSPS TLSASIGDRV TITCRASEGI YHWLAWYQQK PGKAPKLLIY |
| | | KASSLASGAP SRFSGSGSGT DFTLTISSLQ PDDFATYYCQ QYSNYPLTFG |
| | | GGTKLEIKAA AGSGGSGIIH VKGKHLCPSP LFPGPSKPFW VLVVVGGVLA |
| | | CYSLLVTVAF IIFWVRSKRS RLLHSDYMNM TPRRPGPTRK HYQPYAPPRD |
| | | FAAYRSKKVA KKPTNKAPHP KQEPQEINFP DDLPGSNTAA PVQETLHGCQ |
| | | PVTQEDGKES RISVQERQ |
| 317 | hIgGk-VIII_ | MEAPAQLLFL LLLWLPDTTR QVQLVQSGAE VKRPGASVQV SCRASGYSIN |
| | HN1_ | TYYMQWVRQA PGAGLEWMGV INPSGVTSYA QKFQGRVTLT NDTSTNTVYM |
| | spCD28(trun)_ | QLNSLTSADT AVYYCARWAL WGDFGMDVWG KGTLVTVSSG GGGSGGGGSG |
| | CD28_CD40 | GGGSDIQMTQ SPSTLSASIG DRVTITCRAS EGIYHWLAWY QQKPGKAPKL |
| | | LIYKASSLAS GAPSRFSGSG SGTDFTLTIS SLQPDDFATY YCQQYSNYPL |
| | | TFGGGTKLEI KAAAGSGGSG IIHVKGKHLC PSPLFPGPSK PFWVLVVVGG |
| | | VLACYSLLVT VAFIIFWVRS KRSRLLHSDY MNMTPRRPGP TRKHYQPYAP |
| | | PRDFAAYRSK KVAKKPTNKA PHPKQEPQEI NFPDDLPGSN TAAPVQETLH |
| | | GCQPVTQEDG KESRISVQER Q |
| 318 | OSM_M912_ | MGVLLTQRTL LSLVLALLFP SMASMQVQLQ ESGPGLVKPS ETLSLTCTVS |
| | spCD28(trun)_ | GGSVSSGSYY WSWIRQPPGK GLEWIGYIYY SGSTNYNPSL KSRVTISVDT |
| | CD28_CD40 | SKNQFSLKLS SVTAADTAVY YCAREGKNGA FDIWGQGTMV TVSSGGGGSG |
| | CTP251 | GGGSGGGGSD IQMTQSPSSL SASVGDRVTI TCRASQSISS YLNWYQQKPG |
| | | KAPKLLIYAA SSLQSGVPSG FSGSGSGTDF TLTISSLQPE DFATYYCQQS |
| | | YSTPLTFGGG TKVEIKAAAG SGGSGIIHVK GKHLCPSPLF PGPSKPFWVL |
| | | VVVGGVLACY SLLVTVAFII FWVRSKRSRL LHSDYMNMTP RRPGPTRKHY |
| | | QPYAPPRDFA AYRSKKVAKK PTNKAPHPKQ EPQEINFPDD LPGSNTAAPV |
| | | QETLHGCQPV TQEDGKESRI SVQERQ |
| 319 | CD8a_M912_ | MALPVTALLL PLALLLHAAR PQVQLQESGP GLVKPSETLS LTCTVSGGSV |
| | spCD28(trun)_ | SSGSYYWSWI RQPPGKGLEW IGYIYYSGST NYNPSLKSRV TISVDTSKNQ |
| | CD28_CD40 | FSLKLSSVTA ADTAVYYCAR EGKNGAFDIW GQGTMVTVSS GGGGSGGGGS |
| | | GGGGSDIQMT QSPSSLSASV GDRVTITCRA SQSISSYLNW YQQKPGKAPK |
| | | LLIYAASSLQ SGVPSGFSGS GSGTDFTLTI SSLQPEDFAT YYCQQSYSTP |
| | | LTFGGGTKVE IKAAAGSGGS GIIHVKGKHL CPSPLFPGPS KPFWVLVVVG |
| | | GVLACYSLLV TVAFIIFWVR SKRSRLLHSD YMNMTPRRPG PTRKHYQPYA |
| | | PPRDFAAYRS KKVAKKPTNK APHPKQEPQE INFPDDLPGS NTAAPVQETL |
| | | HGCQPVTQED GKESRISVQE RQ |
| 320 | CD2_M912_ | MSFPCKFVAS FLLIFNVSSK GAVSQVQLQE SGPGLVKPSE TLSLTCTVSG |
| | spCD28(trun)_ | GSVSSGSYYW SWIRQPPGKG LEWIGYIYYS GSTNYNPSLK SRVTISVDTS |
| | CD28_CD40 | KNQFSLKLSS VTAADTAVYY CAREGKNGAF DIWGQGTMVT VSSGGGGSGG |
| | | GGSGGGGSDI QMTQSPSSLS ASVGDRVTIT CRASQSISSY LNWYQQKPGK |
| | | APKLLIYAAS SLQSGVPSGF SGSGSGTDFT LTISSLQPED FATYYCQQSY |
| | | STPLTFGGGT KVEIKAAAGS GGSGIIHVKG KHLCPSPLFP GPSKPFWVLV |
| | | VVGGVLACYS LLVTVAFIIF WVRSKRSRLL HSDYMNMTPR RPGPTRKHYQ |
| | | PYAPPRDFAA YRSKKVAKKP TNKAPHPKQE PQEINFPDDL PGSNTAAPVQ |
| | | ETLHGCQPVT QEDGKESRIS VQERQ |
| 321 | IL2_M912_ | MYRMQLLSCI ALSLALVTNS QVQLQESGPG LVKPSETLSL TCTVSGGSVS |
| | spCD28(trun)_ | SGSYYWSWIR QPPGKGLEWI GYIYYSGSTN YNPSLKSRVT ISVDTSKNQF |
| | CD28_CD40 | SLKLSSVTAA DTAVYYCARE GKNGAFDIWG QGTMVTVSSG GGGSGGGGSG |
| | | GGGSDIQMTQ SPSSLSASVG DRVTITCRAS QSISSYLNWY QQKPGKAPKL |
| | | LIYAASSLQS GVPSGFSGSG SGTDFTLTIS SLQPEDFATY YCQQSYSTPL |
| | | TFGGGTKVEI KAAAGSGGSG IIHVKGKHLC PSPLFPGPSK PFWVLVVVGG |
| | | VLACYSLLVT VAFIIFWVRS KRSRLLHSDY MNMTPRRPGP TRKHYQPYAP |
| | | PRDFAAYRSK KVAKKPTNKA PHPKQEPQEI NFPDDLPGSN TAAPVQETLH |
| | | GCQPVTQEDG KESRISVQER Q |
| 322 | GM-CSF_M912_ | MWLQSLLLLG TVACSISQVQ LQESGPGLVK PSETLSLTCT VSGGSVSSGS |
| | spCD28(trun)_ | YYWSWIRQPP GKGLEWIGYI YYSGSTNYNP SLKSRVTISV DTSKNQFSLK |
| | CD28_CD40 | LSSVTAADTA VYYCAREGKN GAFDIWGQGT MVTVSSGGGG SGGGGSGGGG |
| | | SDIQMTQSPS SLSASVGDRV TITCRASQSI SSYLNWYQQK PGKAPKLLIY |
| | | AASSLQSGVP SGFSGSGSGT DFTLTISSLQ PEDFATYYCQ QSYSTPLTFG |
| | | GGTKVEIKAA AGSGGSGIIH VKGKHLCPSP LFPGPSKPFW VLVVVGGVLA |
| | | CYSLLVTVAF IIFWVRSKRS RLLHSDYMNM TPRRPGPTRK HYQPYAPPRD |
| | | FAAYRSKKVA KKPTNKAPHP KQEPQEINFP DDLPGSNTAA PVQETLHGCQ |
| | | PVTQEDGKES RISVQERQ |
| 323 | hIgGk-VIII_ | MEAPAQLLFL LLLWLPDTTR QVQLQESGPG LVKPSETLSL TCTVSGGSVS |
| | M912_ | SGSYYWSWIR QPPGKGLEWI GYIYYSGSTN YNPSLKSRVT ISVDTSKNQF |
| | spCD28(trun)_ | SLKLSSVTAA DTAVYYCARE GKNGAFDIWG QGTMVTVSSG GGGSGGGGSG |
| | CD28_CD40 | GGGSDIQMTQ SPSSLSASVG DRVTITCRAS QSISSYLNWY QQKPGKAPKL |
| | | LIYAASSLQS GVPSGFSGSG SGTDFTLTIS SLQPEDFATY YCQQSYSTPL |
| | | TFGGGTKVEI KAAAGSGGSG IIHVKGKHLC PSPLFPGPSK PFWVLVVVGG |
| | | VLACYSLLVT VAFIIFWVRS KRSRLLHSDY MNMTPRRPGP TRKHYQPYAP |
| | | PRDFAAYRSK KVAKKPTNKA PHPKQEPQEI NFPDDLPGSN TAAPVQETLH |
| | | GCQPVTQEDG KESRISVQER Q |
| 324 | OSM_HuYP218_ | MGVLLTQRTL LSLVLALLFP SMASMEVQLV ESGGGLVQPG GSLRLSCAAS |
| | spCD28(trun)_ | GFDLGFYFYA CWVRQAPGKG LEWVSCIYTA GSGSTYYASW AKGRFTISRD |
| | CD28_CD40 | NSKNTLYLQM NSLRAEDTAV YYCARSTANT RSTYYLNLWG QGTLVTVSSG |
| | CTP252 | GGGSGGGGSG GGGSDIQMTQ SPSSLSASVG DRVTITCQAS QRISSYLSWY |
| | | QQKPGKVPKL LIYGASTLAS GVPSRFSGSG SGTDFTLTIS SLQPEDVATY |
| | | YCQSYAYFDS NNWHAFGGGT KVEIAAAGSG GSGIIHVKGK HLCPSPLFPG |
| | | PSKPFWVLVV VGGVLACYSL LVTVAFIIFW VRSKRSRLLH SDYMNMTPRR |
| | | PGPTRKHYQP YAPPRDFAAY RSKKVAKKPT NKAPHPKQEP QEINFPDDLP |
| | | GSNTAAPVQE TLHGCQPVTQ EDGKESRISV QERQ |
| 325 | CD8a_HuYP218_ | MALPVTALLL PLALLLHAAR PEVQLVESGG GLVQPGGSLR LSCAASGFDL |
| | spCD28(trun)_ | GFYFYACWVR QAPGKGLEWV SCIYTAGSGS TYYASWAKGR FTISRDNSKN |
| | CD28_CD40 | TLYLQMNSLR AEDTAVYYGA RSTANTRSTY YLNLWGQGTL VTVSSGGGGS |
| | | GGGGSGGGGS DIQMTQSPSS LSASVGDRVT ITCQASQRIS SYLSWYQQKP |
| | | GKVPKLLIYG ASTLASGVPS RFSGSGSGTD FTLTISSLQP EDVATYYCQS |
| | | YAYFDSNNWH AFGGGTKVEI AAAGSGGSGI IHVKGKHLCP SPLFPGPSKP |
| | | FWVLVVVGGV LACYSLLVTV AFIIFWVRSK RSRLLHSDYM NMTPRRPGPT |
| | | RKHYQPYAPP RDFAAYRSKK VAKKPTNKAP HPKQEPQEIN FPDDLPGSNT |
| | | AAPVQETLHG CQPVTQEDGK ESRISVQERQ |
| 326 | CD2_HuYP218_ | MSFPCKFVAS FLLIFNVSSK GAVSEVQLVE SGGGLVQPGG SLRLSCAASG |
| | spCD28(trun)_ | FDLGFYFYAC WVRQAPGKGL EWVSCIYTAG SGSTYYASWA KGRFTISRDN |
| | CD28_CD40 | SKNTLYLQMN SLRAEDTAVY YCARSTANTR STYYLNLWGQ GTLVTVSSGG |
| | | GGSGGGGSGG GGSDIQMTQS PSSLSASVGD RVTITCQASQ RISSYLSWYQ |
| | | QKPGKVPKLL IYGASTLASG VPSRFSGSGS GTDFTLTISS LQPEDVATYY |
| | | CQSYAYFDSN NWHAFGGGTK VEIAAAGSGG SGIIHVKGKH LCPSPLFPGP |
| | | SKPFWVLVVV GGVLACYSLL VTVAFIIFWV RSKRSRLLHS DYMNMTPRRP |
| | | GPTRKHYQPY APPRDFAAYR SKKVAKKPTN KAPHPKQEPQ EINFPDDLPG |
| | | SNTAAPVQET LHGCQPVTQE DGKESRISVQ ERQ |
| 327 | IL2_HuYP218_ | MYRMQLLSCI ALSLALVTNS EVQLVESGGG LVQPGGSLRL SCAASGFDLG |
| | spCD28(trun)_ | FYFYACWVRQ APGKGLEWVS GIYTAGSGST YYASWAKGRF TISRDNSKNT |
| | CD28_CD40 | LYLQMNSLRA EDTAVYYCAR STANTRSTYY LNLWGQGTLV TVSSGGGGSG |
| | | GGGSGGGGSD IQMTQSPSSL SASVGDRVTI TCQASQRISS YLSWYQQKPG |
| | | KVPKLLIYGA STLASGVPSR FSGSGSGTDF TLTISSLQPE DVATYYCQSY |
| | | AYFDSNNWHA FGGGTKVEIA AAGSGGSGII HVKGKHLCPS PLFPGPSKPF |
| | | WVLVVVGGVL ACYSLLVTVA FIIFWVRSKR SRLLHSDYMN MTPRRPGPTR |
| | | KHYQPYAPPR DFAAYRSKKV AKKPTNKAPH PKQEPQEINF PDDLPGSNTA |
| | | APVQETLHGC QPVTQEDGKE SRISVQERQ |
| 328 | GM-CSF_ | MWLQSLLLLG TVACSISEVQ LVESGGGLVQ PGGSLRLSCA ASGFDLGFYF |
| | HuYP218_ | YACWVRQAPG KGLEWVSCIY TAGSGSTYYA SWAKGRFTIS RDNSKNTLYL |
| | spCD28(trun)_ | QMNSLRAEDT AVYYCARSTA NTRSTYYLNL WGQGTLVTVS SGGGGSGGGG |
| | CD28_CD40 | SGGGGSDIQM TQSPSSLSAS VGDRVTITCQ ASQRISSYLS WYQQKPGKVP |
| | | KLLIYGASTL ASGVPSRFSG SGSGTDFTLT ISSLQPEDVA TYYCQSYAYF |
| | | DSNNWHAFGG GTKVEIAAAG SGGSGIIHVK GKHLCPSPLF PGPSKPFWVL |
| | | VVVGGVLACY SLLVTVAFII FWVRSKRSRL LHSDYMNMTP RRPGPTRKHY |
| | | QPYAPPRDFA AYRSKKVAKK PTNKAPHPKQ EPQEINFPDD LPGSNTAAPV |
| | | QETLHGCQPV TQEDGKESRI SVQERQ |
| 329 | hIgGk-VIII_ | MEAPAQLLFL LLLWLPDTTR EVQLVESGGG LVQPGGSLRL SCAASGFDLG |
| | HuYP218_ | FYFYACWVRQ APGKGLEWVS GIYTAGSGST YYASWAKGRF TISRDNSKNT |
| | spCD28(trun)_ | LYLQMNSLRA EDTAVYYCAR STANTRSTYY LNLWGQGTLV TVSSGGGGSG |
| | CD28_CD40 | GGGSGGGGSD IQMTQSPSSL SASVGDRVTI TCQASQRISS YLSWYQQKPG |
| | | KVPKLLIYGA STLASGVPSR FSGSGSGTDF TLTISSLQPE DVATYYCQSY |
| | | AYFDSNNWHA FGGGTKVEIA AAGSGGSGII HVKGKHLCPS PLFPGPSKPF |
| | | WVLVVVGGVL ACYSLLVTVA FIIFWVRSKR SRLLHSDYMN MTPRRPGPTR |
| | | KHYQPYAPPR DFAAYRSKKV AKKPTNKAPH PKQEPQEINF PDDLPGSNTA |
| | | APVQETLHGC QPVTQEDGKE SRISVQERQ |
| 330 | OSM_P4_ | MGVLLTQRTL LSLVLALLFP SMASMQVQLQ QSGPGLVTPS QTLSLTCAIS |
| | spCD28(trun)_ | GDSVSSNSAT WNWIRQSPSR GLEWLGRTYY RSKWYNDYAV SVKSRMSINP |
| | CD28_CD40 | DTSKNQFSLQ LNSVTPEDTA VYYCARGMMT YYYGMDVWGQ GTTVTVSSGG |
| | CTP253 | GGSGGGGSGG GGSQPVLTQS SSLSASPGAS ASLTCTLRSG INVGPYRIYW |
| | | YQQKPGSPPQ YLLNYKSDSD KQQGSGVPSR FSGSKDASAN AGVLLISGLR |
| | | SEDEADYYGM IWHSSAAVFG GGTQLTVLSA AAGSGGSGII HVKGKHLCPS |
| | | PLFPGPSKPF WVLVVVGGVL ACYSLLVTVA FIIFWVRSKR SRLLHSDYMN |
| | | MTPRRPGPTR KHYQPYAPPR DFAAYRSKKV AKKPTNKAPH PKQEPQEINF |
| | | PDDLPGSNTA APVQETLHGC QPVTQEDGKE SRISVQERQ |
| 331 | CD8a_P4_ | MALPVTALLL PLALLLHAAR PQVQLQQSGP GLVTPSQTLS LTCAISGDSV |
| | spCD28(trun)_ | SSNSATWNWI RQSPSRGLEW LGRTYYRSKW YNDYAVSVKS RMSINPDTSK |
| | CD28_CD40 | NQFSLQLNSV TPEDTAVYYC ARGMMTYYYG MDVWGQGTTV TVSSGGGGSG |
| | | GGGSGGGGSQ PVLTQSSSLS ASPGASASLT CTLRSGINVG PYRIYWYQQK |
| | | PGSPPQYLLN YKSDSDKQQG SGVPSRFSGS KDASANAGVL LISGLRSEDE |
| | | ADYYCMIWHS SAAVFGGGTQ LTVLSAAAGS GGSGIIHVKG KHLCPSPLFP |
| | | GPSKPFWVLV VVGGVLACYS LLVTVAFIIF WVRSKRSRLL HSDYMNMTPR |
| | | RPGPTRKHYQ PYAPPRDFAA YRSKKVAKKP TNKAPHPKQE PQEINFPDDL |
| | | PGSNTAAPVQ ETLHGCQPVT QEDGKESRIS VQERQ |
| 332 | CD2_P4_ | MSFPCKFVAS FLLIFNVSSK GAVSQVQLQQ SGPGLVTPSQ TLSLTCAISG |
| | spCD28(trun)_ | DSVSSNSATW NWIRQSPSRG LEWLGRTYYR SKWYNDYAVS VKSRMSINPD |
| | CD28_CD40 | TSKNQFSLQL NSVTPEDTAV YYCARGMMTY YYGMDVWGQG TTVTVSSGGG |
| | | GSGGGGSGGG GSQPVLTQSS SLSASPGASA SLTCTLRSGI NVGPYRIYWY |
| | | QQKPGSPPQY LLNYKSDSDK QQGSGVPSRF SGSKDASANA GVLLISGLRS |
| | | EDEADYYCMI WHSSAAVFGG GTQLTVLSAA AGSGGSGIIH VKGKHLCPSP |
| | | LFPGPSKPFW VLVVVGGVLA CYSLLVTVAF IIFWVRSKRS RLLHSDYMNM |
| | | TPRRPGPTRK HYQPYAPPRD FAAYRSKKVA KKPTNKAPHP KQEPQEINFP |
| | | DDLPGSNTAA PVQETLHGCQ PVTQEDGKES RISVQERQ |
| 333 | IL2_P4_ | MYRMQLLSCI ALSLALVTNS QVQLQQSGPG LVTPSQTLSL TCAISGDSVS |
| | spCD28(trun)_ | SNSATWNWIR QSPSRGLEWL GRTYYRSKWY NDYAVSVKSR MSINPDTSKN |
| | CD28_CD40 | QFSLQLNSVT PEDTAVYYCA RGMMTYYYGM DVWGQGTTVT VSSGGGGSGG |
| | | GGSGGGGSQP VLTQSSSLSA SPGASASLTC TLRSGINVGP YRIYWYQQKP |
| | | GSPPQYLLNY KSDSDKQQGS GVPSRFSGSK DASANAGVLL ISGLRSEDEA |
| | | DYYCMIWHSS AAVFGGGTQL TVLSAAAGSG GSGIIHVKGK HLCPSPLFPG |
| | | PSKPFWVLVV VGGVLACYSL LVTVAFIIFW VRSKRSRLLH SDYMNMTPRR |
| | | PGPTRKHYQP YAPPRDFAAY RSKKVAKKPT NKAPHPKQEP QEINFPDDLP |
| | | GSNTAAPVQE TLHGCQPVTQ EDGKESRISV QERQ |
| 334 | GM-CSF_P4_ | MWLQSLLLLG TVACSISQVQ LQQSGPGLVT PSQTLSLTCA ISGDSVSSNS |
| | spCD28(trun)_ | ATWNWIRQSP SRGLEWLGRT YYRSKWYNDY AVSVKSRMSI NPDTSKNQFS |
| | CD28_CD40 | LQLNSVTPED TAVYYCARGM MTYYYGMDVW GQGTTVTVSS GGGGSGGGGS |
| | | GGGGSQPVLT QSSSLSASPG ASASLTCTLR SGINVGPYRI YWYQQKPGSP |
| | | PQYLLNYKSD SDKQQGSGVP SRFSGSKDAS ANAGVLLISG LRSEDEADYY |
| | | CMIWHSSAAV FGGGTQLTVL SAAAGSGGSG IIHVKGKHLC PSPLFPGPSK |
| | | PFWVLVVVGG VLACYSLLVT VAFIIFWVRS KRSRLLHSDY MNMTPRRPGP |
| | | TRKHYQPYAP PRDFAAYRSK KVAKKPTNKA PHPKQEPQEI NFPDDLPGSN |
| | | TAAPVQETLH GCQPVTQEDG KESRISVQER Q |
| 335 | hIgGk-VIII_ | MEAPAQLLFL LLLWLPDTTR QVQLQQSGPG LVTPSQTLSL TCAISGDSVS |
| | P4_ | SNSATWNWIR QSPSRGLEWL GRTYYRSKWY NDYAVSVKSR MSINPDTSKN |
| | spCD28(trun)_ | QFSLQLNSVT PEDTAVYYCA RGMMTYYYGM DVWGQGTTVT VSSGGGGSGG |
| | CD28_CD40 | GGSGGGGSQP VLTQSSSLSA SPGASASLTC TLRSGINVGP YRIYWYQQKP |
| | | GSPPQYLLNY KSDSDKQQGS GVPSRFSGSK DASANAGVLL ISGLRSEDEA |
| | | DYYCMIWHSS AAVFGGGTQL TVLSAAAGSG GSGIIHVKGK HLCPSPLFPG |
| | | PSKPFWVLVV VGGVLACYSL LVTVAFIIFW VRSKRSRLLH SDYMNMTPRR |
| | | PGPTRKHYQP YAPPRDFAAY RSKKVAKKPT NKAPHPKQEP QEINFPDDLP |
| | | GSNTAAPVQE TLHGCQPVTQ EDGKESRISV QERQ |
| 336 | NTRK1 IC domain | NKCGRRNKFG INRPAVLAPE DGLAMSLHFM TLGGSSLSPT EGKGSGLQGH |
| | | IIENPQYFSD ACVHHIKRRD IVLKWELGEG AFGKVFLAEC HNLLPEQDKM |
| | | LVAVKALKEA SESARQDFQR EAELLTMLQH QHIVRFFGVC TEGRPLLMVF |
| | | EYMRHGDLNR FLRSHGPDAK LLAGGEDVAP GPLGLGQLLA VASQVAAGMV |
| | | YLAGLHFVHR DLATRNCLVG QGLVVKIGDF GMSRDIYSTD YYRVGGRTML |
| | | PIRWMPPESI LYRKFTTESD VWSFGVVLWE IFTYGKQPWY QLSNTEAIDC |
| | | ITQGRELERP RACPPEVYAI MRGCWQREPQ QRHSIKDVHA RLQALAQAPP |
| | | VYLDVLG |
| 337 | ICOS | MKSGLWYFFLFLCRIKVLTGEINGSANYEMFIFHNGGVQILCKYPDIVQQ |
| | | FKMQLLKGGQILCDLTKTKGSGNTVSIKSLKFCHSQLSNNSVSFFLYNLD |
| | | HSHANYYFCNLSIFDPPPFKVTLTGGYLHIYESQLCCQLKFWLPIGCAAF |
| | | VVVCILGCILICWLTKKKYSSSVHDPNGEYMFMRAVNTAKKSRLTDVTL |
| 338 | ICOS IC domain | CWLTKKKYSS SVHDPNGEYM FMRAVNTAKK SRLTDVTL |
| 339 | mouse CD2 | RDNETIWGVL GHGITLNIPN FQMTDDIDEV RWVRRGTLVA EFKRKKPPFL |
| | | ISETYEVLAN GSLKIKKPMM RNDSGTYNVM VYGTNGMTRL EKDLDVRILE |
| | | RVSKPMIHWE CPNTTLTCAV LQGTDFELKL YQGETLLNSL PQKNMSYQWT |
| | | NLNAPFKCEA INPVSKESKM EVVNCPEKGL SFYVTVGVGA GGLLLVLLVA |
| | | LFIFCICKRR KRNRRRKDEE LEIKASRTST VERGPKPHST PAAAAQNSVA |
| | | LQAPPPPGHH LQTPGHRPLP PGHRTREHQQ KKRPPPSGTQ IHQQKGPPLP |
| | | RPRVQPKPPC GSGDGVSLPP PN |
| 340 | mouse CD2 IC | KRRKRNRRRK DEELEIKASR TSTVERGPKP HSTPAAAAQN SVALQAPPPP |
| | domain | GHHLQTPGHR PLPPGHRTRE HQQKKRPPPS GTQIHQQKGP PLPRPRVQPK |
| | | PPCGSGDGVS LPPPN |
| 341 | CD137 IC domain | KRGRKKLLYI FKQPFMRPVQ TTQEEDGCSC RFPEEEEGGC EL |
| 342 | DAP10 IC domain | LCARPRRSPA QEDGKVYINM PGRG |
| 343 | CD134 IC domain | ALYLLRRDQR LPPDAHKPPG GGSFRTPIQE EQADAHSTLA KI |
| 344 | hMFE23. | MGVLLTQRTL LSLVLALLFP SMASMQVKLE QSGAEVVKPG ASVKLSCKAS |
| | TMtCD28.NTRK1 | GFNIKDSYMH WLRQGPGQRL EWIGWIDPEN GDTEYAPKFQ GKATFTTDTS |
| | CTP313 | ANTAYLGLSS LRPEDTAVYY CNEGTPTGPY YFDYWGQGTL VTVSSGGGGS |
| | | GGGGSGGGGS ENVLTQSPSS MSASVGDRVN IACSASSSVS YMHWFQQKPG |
| | | KSPKLWIYST SNLASGVPSR FSGSGSGTDY SLTISSMQPE DAATYYCQQR |
| | | SSYPLTFGGG TKLEIKAAAG SGGSGILVKQ SPMLVAYDNA VNLSCKYSYN |
| | | LFSREFRASL HKGLDSAVEV CVVYGNYSQQ LQVYSKTGFN CDGKLGNESV |
| | | TFYLQNLYVN QTDIYFCKIE VMYPPPYLDN EKSNGTIIHV KGKHLCPSPL |
| | | FPGPSKPFWV LVVVGGVLAC YSLLVTVAFI IFWVNKCGRR NKFGINRPAV |
| | | LAPEDGLAMS LHFMTLGGSS LSPTEGKGSG LQGHIIENPQ YFSDACVHHI |
| | | KRRDIVLKWE LGEGAFGKVF LAECHNLLPE QDKMLVAVKA LKEASESARQ |
| | | DFQREAELLT MLQHQHIVRF FGVCTEGRPL LMVFEYMRHG DLNRFLRSHG |
| | | PDAKLLAGGE DVAPGPLGLG QLLAVASQVA AGMVYLAGLH FVHRDLATRN |
| | | CLVGQGLVVK IGDFGMSRDI YSTDYYRVGG RTMLPIRWMP PESILYRKFT |
| | | TESDVWSFGV VLWEIFTYGK QPWYQLSNTE AIDCITQGRE LERPRACPPE |
| | | VYAIMRGCWQ REPQQRHSIK DVHARLQALA QAPPVYLDVLG |
| 345 | hMFE23. | MGVLLTQRTL LSLVLALLFP SMASMQVKLE QSGAEVVKPG ASVKLSCKAS |
| | TMFCD28. | GFNIKDSYMH WLRQGPGQRL EWIGWIDPEN GDTEYAPKFQ GKATFTTDTS |
| | NTRK1.CD40 | ANTAYLGLSS LRPEDTAVYY CNEGTPTGPY YFDYWGQGTL VTVSSGGGGS |
| | CTP314 | GGGGSGGGGS ENVLTQSPSS MSASVGDRVN IACSASSSVS YMHWFQQKPG |
| | | KSPKLWIYST SNLASGVPSR FSGSGSGTDY SLTISSMQPE DAATYYCQQR |
| | | SSYPLTFGGG TKLEIKAAAG SGGSGILVKQ SPMLVAYDNA VNLSCKYSYN |
| | | LFSREFRASL HKGLDSAVEV CVVYGNYSQQ LQVYSKTGFN CDGKLGNESV |
| | | TFYLQNLYVN QTDIYFCKIE VMYPPPYLDN EKSNGTIIHV KGKHLCPSPL |
| | | FPGPSKPFWV LVVVGGVLAC YSLLVTVAFI IFWVNKCGRR NKFGINRPAV |
| | | LAPEDGLAMS LHFMTLGGSS LSPTEGKGSG LQGHIIENPQ YFSDACVHHI |
| | | KRRDIVLKWE LGEGAFGKVF LAECHNLLPE QDKMLVAVKA LKEASESARQ |
| | | DFQREAELLT MLQHQHIVRF FGVCTEGRPL LMVFEYMRHG DLNRFLRSHG |
| | | PDAKLLAGGE DVAPGPLGLG QLLAVASQVA AGMVYLAGLH FVHRDLATRN |
| | | CLVGQGLVVK IGDFGMSRDI YSTDYYRVGG RTMLPIRWMP PESILYRKFT |
| | | TESDVWSFGV VLWEIFTYGK QPWYQLSNTE AIDCITQGRE LERPRACPPE |
| | | VYAIMRGCWQ REPQQRHSIK DVHARLQALA QAPPVYLDVL GKKVAKKPTN |
| | | KAPHPKQEPQ EINFPDDLPG SNTAAPVQET LHGCQPVTQE DGKESRISVQ |
| | | ERQ |
| 346 | hMFE23.CD28. | MGVLLTQRTL LSLVLALLFP SMASMQVKLE QSGAEVVKPG ASVKLSCKAS |
| | tICNTRK1 | GFNIKDSYMH WLRQGPGQRL EWIGWIDPEN GDTEYAPKFQ GKATFTTDTS |
| | CTP315 | ANTAYLGLSS LRPEDTAVYY CNEGTPTGPY YFDYWGQGTL VTVSSGGGGS |
| | | GGGGSGGGGS ENVLTQSPSS MSASVGDRVN IACSASSSVS YMHWFQQKPG |
| | | KSPKLWIYST SNLASGVPSR FSGSGSGTDY SLTISSMQPE DAATYYCQQR |
| | | SSYPLTFGGG TKLEIKAAAG SGGSGILVKQ SPMLVAYDNA VNLSCKYSYN |
| | | LFSREFRASL HKGLDSAVEV CVVYGNYSQQ LQVYSKTGFN CDGKLGNESV |
| | | TFYLQNLYVN QTDIYFCKIE VMYPPPYLDN EKSNGTIIHV KGKHLCPSPL |
| | | FPGPSKPFWV LVVVGGVLAC YSLLVTVAFI IFWVRSKRSR LLHSDYMNMT |
| | | PRRPGPTRKH YQPYAPPRDF AAYRSNKCGR RNKFGINRPA VLAPEDGLAM |
| | | SLHFMTLGGS SLSPTEGKGS GLQGHIIENP QYFSDACVHH IKRRDIVLKW |
| | | ELGEGAFGKV FLAECHNLLP EQDKMLVAVK ALKEASESAR QDFQREAELL |
| | | TMLQHQHIVR FFGVCTEGRP LLMVFEYMRH GDLNRFLRSH GPDAKLLAGG |
| | | EDVAPGPLGL GQLLAVASQV AAGMVYLAGL HFVHRDLATR NCLVGQGLVV |
| | | KIGDFGMSRD IYSTDYYRVG GRTMLPIRWM PPESILYRKF TTESDVWSFG |
| | | VVLWEIFTYG KQPWYQLSNT EAIDCITQGR ELERPRACPP EVYAIMRGCW |
| | | QREPQQRHSI KDVHARLQAL AQAPPVYLDV LG |
| 347 | hMFE23.CD28. | MGVLLTQRTL LSLVLALLFP SMASMQVKLE QSGAEVVKPG ASVKLSCKAS |
| | tICNTRK1.CD40 | GFNIKDSYMH WLRQGPGQRL EWIGWIDPEN GDTEYAPKFQ GKATFTTDTS |
| | CTP316 | ANTAYLGLSS LRPEDTAVYY CNEGTPTGPY YFDYWGQGTL VTVSSGGGGS |
| | | GGGGSGGGGS ENVLTQSPSS MSASVGDRVN IACSASSSVS YMHWFQQKPG |
| | | KSPKLWIYST SNLASGVPSR FSGSGSGTDY SLTISSMQPE DAATYYCQQR |
| | | SSYPLTFGGG TKLEIKAAAG SGGSGILVKQ SPMLVAYDNA VNLSCKYSYN |
| | | LFSREFRASL HKGLDSAVEV CVVYGNYSQQ LQVYSKTGFN CDGKLGNESV |
| | | TFYLQNLYVN QTDIYFCKIE VMYPPPYLDN EKSNGTIIHV KGKHLCPSPL |
| | | FPGPSKPFWV LVVVGGVLAC YSLLVTVAFI IFWVRSKRSR LLHSDYMNMT |
| | | PRRPGPTRKH YQPYAPPRDF AAYRSNKCGR RNKFGINRPA VLAPEDGLAM |
| | | SLHFMTLGGS SLSPTEGKGS GLQGHIIENP QYFSDACVHH IKRRDIVLKW |
| | | ELGEGAFGKV FLAECHNLLP EQDKMLVAVK ALKEASESAR QDFQREAELL |
| | | TMLQHQHIVR FFGVCTEGRP LLMVFEYMRH GDLNRFLRSH GPDAKLLAGG |
| | | EDVAPGPLGL GQLLAVASQV AAGMVYLAGL HFVHRDLATR NCLVGQGLVV |
| | | KIGDFGMSRD IYSTDYYRVG GRTMLPIRWM PPESILYRKF TTESDVWSFG |
| | | VVLWEIFTYG KQPWYQLSNT EAIDCITQGR ELERPRACPP EVYAIMRGCW |
| | | QREPQQRHSI KDVHARLQAL AQAPPVYLDV LGKKVAKKPT NKAPHPKQEP |
| | | QEINFPDDLP GSNTAAPVQE TLHGCQPVTQ EDGKESRISV QERQ |
| 348 | hMFE23. | MGVLLTQRTL LSLVLALLFP SMASMQVKLE QSGAEVVKPG ASVKLSCKAS |
| | ICOS.CD40 | GFNIKDSYMH WLRQGPGQRL EWIGWIDPEN GDTEYAPKFQ GKATFTTDTS |
| | CTP317 | ANTAYLGLSS LRPEDTAVYY CNEGTPTGPY YFDYWGQGTL VTVSSGGGGS |
| | | GGGGSGGGGS ENVLTQSPSS MSASVGDRVN IACSASSSVS YMHWFQQKPG |
| | | KSPKLWIYST SNLASGVPSR FSGSGSGTDY SLTISSMQPE DAATYYCQQR |
| | | SSYPLTFGGG TKLEIKAAAG SGGSGGEING SANYEMFIFH NGGVQILCKY |
| | | PDIVQQFKMQ LLKGGQILCD LTKTKGSGNT VSIKSLKFCH SQLSNNSVSF |
| | | FLYNLDHSHA NYYFCNLSIF DPPPFKVTLT GGYLHIYESQ LCCQLKFWLP |
| | | IGCAAFVVVC ILGCILICWL TKKKYSSSVH DPNGEYMFMR AVNTAKKSRL |
| | | TDVTLKKVAK KPTNKAPHPK QEPQEINFPD DLPGSNTAAP VQETLHGCQP |
| | | VTQEDGKESR ISVQERQ |
| 349 | hMFE23. | MGVLLTQRTL LSLVLALLFP SMASMQVKLE QSGAEVVKPG ASVKLSCKAS |
| | CD28.ICOS | GFNIKDSYMH WLRQGPGQRL EWIGWIDPEN GDTEYAPKFQ GKATFTTDTS |
| | CTP318 | ANTAYLGLSS LRPEDTAVYY CNEGTPTGPY YFDYWGQGTL VTVSSGGGGS |
| | | GGGGSGGGGS ENVLTQSPSS MSASVGDRVN IACSASSSVS YMHWFQQKPG |
| | | KSPKLWIYST SNLASGVPSR FSGSGSGTDY SLTISSMQPE DAATYYCQQR |
| | | SSYPLTFGGG TKLEIKAAAG SGGSGILVKQ SPMLVAYDNA VNLSCKYSYN |
| | | LFSREFRASL HKGLDSAVEV CVVYGNYSQQ LQVYSKTGFN CDGKLGNESV |
| | | TFYLQNLYVN QTDIYFCKIE VMYPPPYLDN EKSNGTIIHV KGKHLCPSPL |
| | | FPGPSKPFWV LVVVGGVLAC YSLLVTVAFI IFWVRSKRSR LLHSDYMNMT |
| | | PRRPGPTRKH YQPYAPPRDF AAYRSCWLTK KKYSSSVHDP NGEYMFMRAV |
| | | NTAKKSRLTD VTL |
| 350 | hMFE23.CD28. | MGVLLTQRTL LSLVLALLFP SMASMQVKLE QSGAEVVKPG ASVKLSCKAS |
| | ICOS.CD40 | GFNIKDSYMH WLRQGPGQRL EWIGWIDPEN GDTEYAPKFQ GKATFTTDTS |
| | CTP319 | ANTAYLGLSS LRPEDTAVYY CNEGTPTGPY YFDYWGQGTL VTVSSGGGGS |
| | | GGGGSGGGGS ENVLTQSPSS MSASVGDRVN IACSASSSVS YMHWFQQKPG |
| | | KSPKLWIYST SNLASGVPSR FSGSGSGTDY SLTISSMQPE DAATYYCQQR |
| | | SSYPLTFGGG TKLEIKAAAG SGGSGILVKQ SPMLVAYDNA VNLSCKYSYN |
| | | LFSREFRASL HKGLDSAVEV CVVYGNYSQQ LQVYSKTGFN CDGKLGNESV |
| | | TFYLQNLYVN QTDIYFCKIE VMYPPPYLDN EKSNGTIIHV KGKHLCPSPL |
| | | FPGPSKPFWV LVVVGGVLAC YSLLVTVAFI IFWVRSKRSR LLHSDYMNMT |
| | | PRRPGPTRKH YQPYAPPRDF AAYRSCWLTK KKYSSSVHDP NGEYMFMRAV |
| | | NTAKKSRLTD VTLKKVAKKP TNKAPHPKQE PQEINFPDDL PGSNTAAPVQ |
| | | ETLHGCQPVT QEDGKESRIS VQERQ |
| 351 | hMFE23.CD2.CD40 | MGVLLTQRTL LSLVLALLFP SMASMQVKLE QSGAEVVKPG ASVKLSCKAS |
| | CTP320 | GFNIKDSYMH WLRQGPGQRL EWIGWIDPEN GDTEYAPKFQ GKATFTTDTS |
| | | ANTAYLGLSS LRPEDTAVYY CNEGTPTGPY YFDYWGQGTL VTVSSGGGGS |
| | | GGGGSGGGGS ENVLTQSPSS MSASVGDRVN IACSASSSVS YMHWFQQKPG |
| | | KSPKLWIYST SNLASGVPSR FSGSGSGTDY SLTISSMQPE DAATYYCQQR |
| | | SSYPLTFGGG TKLEIKAAAG SGGSGRDNET IWGVLGHGIT LNIPNFQMTD |
| | | DIDEVRWVRR GTLVAEFKRK KPPFLISETY EVLANGSLKI KKPMMRNDSG |
| | | TYNVMVYGTN GMTRLEKDLD VRILERVSKP MIHWECPNTT LTCAVLQGTD |
| | | FELKLYQGET LLNSLPQKNM SYQWTNLNAP FKCEAINPVS KESKMEVVNC |
| | | PEKGLSFYVT VGVGAGGLLL VLLVALFIFC ICKRRKRNRR RKDEELEIKA |
| | | SRTSTVERGP KPHSTPAAAA QNSVALQAPP PPGHHLQTPG HRPLPPGHRT |
| | | REHQQKKRPP PSGTQIHQQK GPPLPRPRVQ PKPPCGSGDG VSLPPPNKKV |
| | | AKKPTNKAPH PKQEPQEINF PDDLPGSNTA APVQETLHGC QPVTQEDGKE |
| | | SRISVQERQ |
| 352 | hMFE23. | MGVLLTQRTL LSLVLALLFP SMASMQVKLE QSGAEVVKPG ASVKLSCKAS |
| | CD28.CD2 | GFNIKDSYMH WLRQGPGQRL EWIGWIDPEN GDTEYAPKFQ GKATFTTDTS |
| | CTP321 | ANTAYLGLSS LRPEDTAVYY CNEGTPTGPY YFDYWGQGTL VTVSSGGGGS |
| | | GGGGSGGGGS ENVLTQSPSS MSASVGDRVN IACSASSSVS YMHWFQQKPG |
| | | KSPKLWIYST SNLASGVPSR FSGSGSGTDY SLTISSMQPE DAATYYCQQR |
| | | SSYPLTFGGG TKLEIKAAAG SGGSGILVKQ SPMLVAYDNA VNLSCKYSYN |
| | | LFSREFRASL HKGLDSAVEV CVVYGNYSQQ LQVYSKTGFN CDGKLGNESV |
| | | TFYLQNLYVN QTDIYFCKIE VMYPPPYLDN EKSNGTIIHV KGKHLCPSPL |
| | | FPGPSKPFWV LVVVGGVLAC YSLLVTVAFI IFWVRSKRSR LLHSDYMNMT |
| | | PRRPGPTRKH YQPYAPPRDF AAYRSKRRKR NRRRKDEELE IKASRTSTVE |
| | | RGPKPHSTPA AAAQNSVALQ APPPPGHHLQ TPGHRPLPPG HRTREHQQKK |
| | | RPPPSGTQIH QQKGPPLPRP RVQPKPPCGS GDGVSLPPPN |
| 353 | hMFE23.CD28. | MGVLLTQRTL LSLVLALLFP SMASMQVKLE QSGAEVVKPG ASVKLSCKAS |
| | CD40.CD2 | GFNIKDSYMH WLRQGPGQRL EWIGWIDPEN GDTEYAPKFQ GKATFTTDTS |
| | CTP322 | ANTAYLGLSS LRPEDTAVYY CNEGTPTGPY YFDYWGQGTL VTVSSGGGGS |
| | | GGGGSGGGGS ENVLTQSPSS MSASVGDRVN IACSASSSVS YMHWFQQKPG |
| | | KSPKLWIYST SNLASGVPSR FSGSGSGTDY SLTISSMQPE DAATYYCQQR |
| | | SSYPLTFGGG TKLEIKAAAG SGGSGILVKQ SPMLVAYDNA VNLSCKYSYN |
| | | LFSREFRASL HKGLDSAVEV CVVYGNYSQQ LQVYSKTGFN CDGKLGNESV |
| | | TFYLQNLYVN QTDIYFCKIE VMYPPPYLDN EKSNGTIIHV KGKHLCPSPL |
| | | FPGPSKPFWV LVVVGGVLAC YSLLVTVAFI IFWVRSKRSR LLHSDYMNMT |
| | | PRRPGPTRKH YQPYAPPRDF AAYRSKKVAK KPTNKAPHPK QEPQEINFPD |
| | | DLPGSNTAAP VQETLHGCQP VTQEDGKESR ISVQERQKRR KRNRRRKDEE |
| | | LEIKASRTST VERGPKPHST PAAAAQNSVA LQAPPPPGHH LQTPGHRPLP |
| | | PGHRTREHQQ KKRPPPSGTQ IHQQKGPPLP RPRVQPKPPC GSGDGVSLPP |
| | | PN |
| 354 | hMFE23. | MGVLLTQRTL LSLVLALLFP SMASMQVKLE QSGAEVVKPG ASVKLSCKAS |
| | CD28.CD137 | GFNIKDSYMH WLRQGPGQRL EWIGWIDPEN GDTEYAPKFQ GKATFTTDTS |
| | CTP323 | ANTAYLGLSS LRPEDTAVYY CNEGTPTGPY YFDYWGQGTL VTVSSGGGGS |
| | | GGGGSGGGGS ENVLTQSPSS MSASVGDRVN IACSASSSVS YMHWFQQKPG |
| | | KSPKLWIYST SNLASGVPSR FSGSGSGTDY SLTISSMQPE DAATYYCQQR |
| | | SSYPLTFGGG TKLEIKAAAG SGGSGILVKQ SPMLVAYDNA VNLSCKYSYN |
| | | LFSREFRASL HKGLDSAVEV CVVYGNYSQQ LQVYSKTGFN CDGKLGNESV |
| | | TFYLQNLYVN QTDIYFCKIE VMYPPPYLDN EKSNGTIIHV KGKHLCPSPL |
| | | FPGPSKPFWV LVVVGGVLAC YSLLVTVAFI IFWVRSKRSR LLHSDYMNMT |
| | | PRRPGPTRKH YQPYAPPRDF AAYRSKRGRK KLLYIFKQPF MRPVQTTQEE |
| | | DGCSCRFPEE EEGGCEL |
| 355 | hMFE23.CD28. | MGVLLTQRTL LSLVLALLFP SMASMQVKLE QSGAEVVKPG ASVKLSCKAS |
| | CD40.CD137 | GFNIKDSYMH WLRQGPGQRL EWIGWIDPEN GDTEYAPKFQ GKATFTTDTS |
| | CTP324 | ANTAYLGLSS LRPEDTAVYY CNEGTPTGPY YFDYWGQGTL VTVSSGGGGS |
| | | GGGGSGGGGS ENVLTQSPSS MSASVGDRVN IACSASSSVS YMHWFQQKPG |
| | | KSPKLWIYST SNLASGVPSR FSGSGSGTDY SLTISSMQPE DAATYYCQQR |
| | | SSYPLTFGGG TKLEIKAAAG SGGSGILVKQ SPMLVAYDNA VNLSCKYSYN |
| | | LFSREFRASL HKGLDSAVEV CVVYGNYSQQ LQVYSKTGFN CDGKLGNESV |
| | | TFYLQNLYVN QTDIYFCKIE VMYPPPYLDN EKSNGTIIHV KGKHLCPSPL |
| | | FPGPSKPFWV LVVVGGVLAC YSLLVTVAFI IFWVRSKRSR LLHSDYMNMT |
| | | PRRPGPTRKH YQPYAPPRDF AAYRSKKVAK KPTNKAPHPK QEPQEINFPD |
| | | DLPGSNTAAP VQETLHGCQP VTQEDGKESR ISVQERQKRG RKKLLYIFKQ |
| | | PFMRPVQTTQ EEDGCSCRFP EEEEGGCEL |
| 356 | hMFE23.CD28. | MGVLLTQRTL LSLVLALLFP SMASMQVKLE QSGAEVVKPG ASVKLSCKAS |
| | DAP10 | GFNIKDSYMH WLRQGPGQRL EWIGWIDPEN GDTEYAPKFQ GKATFTTDTS |
| | CTP325 | ANTAYLGLSS LRPEDTAVYY CNEGTPTGPY YFDYWGQGTL VTVSSGGGGS |
| | | GGGGSGGGGS ENVLTQSPSS MSASVGDRVN IACSASSSVS YMHWFQQKPG |
| | | KSPKLWIYST SNLASGVPSR FSGSGSGTDY SLTISSMQPE DAATYYCQQR |
| | | SSYPLTFGGG TKLEIKAAAG SGGSGILVKQ SPMLVAYDNA VNLSCKYSYN |
| | | LFSREFRASL HKGLDSAVEV CVVYGNYSQQ LQVYSKTGFN CDGKLGNESV |
| | | TFYLQNLYVN QTDIYFCKIE VMYPPPYLDN EKSNGTIIHV KGKHLCPSPL |
| | | FPGPSKPFWV LVVVGGVLAC YSLLVTVAFI IFWVRSKRSR LLHSDYMNMT |
| | | PRRPGPTRKH YQPYAPPRDF AAYRSLCARP RRSPAQEDGK VYINMPGRG |
| 357 | hMFE23.CD28. | MGVLLTQRTL LSLVLALLFP SMASMQVKLE QSGAEVVKPG ASVKLSCKAS |
| | CD40.DAP10 | GFNIKDSYMH WLRQGPGQRL EWIGWIDPEN GDTEYAPKFQ GKATFTTDTS |
| | CTP326 | ANTAYLGLSS LRPEDTAVYY CNEGTPTGPY YFDYWGQGTL VTVSSGGGGS |
| | | GGGGSGGGGS ENVLTQSPSS MSASVGDRVN IACSASSSVS YMHWFQQKPG |
| | | KSPKLWIYST SNLASGVPSR FSGSGSGTDY SLTISSMQPE DAATYYCQQR |
| | | SSYPLTFGGG TKLEIKAAAG SGGSGILVKQ SPMLVAYDNA VNLSCKYSYN |
| | | LFSREFRASL HKGLDSAVEV CVVYGNYSQQ LQVYSKTGFN CDGKLGNESV |
| | | TFYLQNLYVN QTDIYFCKIE VMYPPPYLDN EKSNGTIIHV KGKHLCPSPL |
| | | FPGPSKPFWV LVVVGGVLAC YSLLVTVAFI IFWVRSKRSR LLHSDYMNMT |
| | | PRRPGPTRKH YQPYAPPRDF AAYRSKKVAK KPTNKAPHPK QEPQEINFPD |
| | | DLPGSNTAAP VQETLHGCQP VTQEDGKESR ISVQERQLCA RPRRSPAQED |
| | | GKVYINMPGR G |
| 358 | hMFE23.CD28. | MGVLLTQRTL LSLVLALLFP SMASMQVKLE QSGAEVVKPG ASVKLSCKAS |
| | CD134 | GFNIKDSYMH WLRQGPGQRL EWIGWIDPEN GDTEYAPKFQ GKATFTTDTS |
| | CTP327 | ANTAYLGLSS LRPEDTAVYY CNEGTPTGPY YFDYWGQGTL VTVSSGGGGS |
| | | GGGGSGGGGS ENVLTQSPSS MSASVGDRVN IACSASSSVS YMHWFQQKPG |
| | | KSPKLWIYST SNLASGVPSR FSGSGSGTDY SLTISSMQPE DAATYYCQQR |
| | | SSYPLTFGGG TKLEIKAAAG SGGSGILVKQ SPMLVAYDNA VNLSCKYSYN |
| | | LFSREFRASL HKGLDSAVEV CVVYGNYSQQ LQVYSKTGFN CDGKLGNESV |
| | | TFYLQNLYVN QTDIYFCKIE VMYPPPYLDN EKSNGTIIHV KGKHLCPSPL |
| | | FPGPSKPFWV LVVVGGVLAC YSLLVTVAFI IFWVRSKRSR LLHSDYMNMT |
| | | PRRPGPTRKH YQPYAPPRDF AAYRSALYLL RRDQRLPPDA HKPPGGGSFR |
| | | TPIQEEQADA HSTLAKI |
| 359 | hMFE23.CD28. | MGVLLTQRTL LSLVLALLFP SMASMQVKLE QSGAEVVKPG ASVKLSCKAS |
| | CD40.CD134 | GFNIKDSYMH WLRQGPGQRL EWIGWIDPEN GDTEYAPKFQ GKATFTTDTS |
| | CTP328 | ANTAYLGLSS LRPEDTAVYY CNEGTPTGPY YFDYWGQGTL VTVSSGGGGS |
| | | GGGGSGGGGS ENVLTQSPSS MSASVGDRVN IACSASSSVS YMHWFQQKPG |
| | | KSPKLWIYST SNLASGVPSR FSGSGSGTDY SLTISSMQPE DAATYYCQQR |
| | | SSYPLTFGGG TKLEIKAAAG SGGSGILVKQ SPMLVAYDNA VNLSCKYSYN |
| | | LFSREFRASL HKGLDSAVEV CVVYGNYSQQ LQVYSKTGFN CDGKLGNESV |
| | | TFYLQNLYVN QTDIYFCKIE VMYPPPYLDN EKSNGTIIHV KGKHLCPSPL |
| | | FPGPSKPFWV LVVVGGVLAC YSLLVTVAFI IFWVRSKRSR LLHSDYMNMT |
| | | PRRPGPTRKH YQPYAPPRDF AAYRSKKVAK KPTNKAPHPK QEPQEINFPD |
| | | DLPGSNTAAP VQETLHGCQP VTQEDGKESR ISVQERQALY LLRRDQRLPP |
| | | DAHKPPGGGS FRTPIQEEQA DAHSTLAKI |
| 360 | PD1_PDl_ | MQIPQAPWPV VWAVLQLGWR PGWFLDSPDR PWNPPTFSPA LLVVTEGDNA |
| | sCD28TM_CD28_ | TFTCSFSNTS ESFVLNWYRM SPSNQTDKLA AFPEDRSQPG QDCRFRVTQL |
| | CD40 | PNGRDFHMSV VRARRNDSGT YLCGAISLAP KAQIKESLRA ELRVTERRAE |
| | (monomeric) | VPTAHPSPSP RPAGQFWVLV VVGGVLACYS LLVTVAFIIF WVRSKRSRLL |
| | CTP189 | HSDYMNMTPR RPGPTRKHYQ PYAPPRDFAA YRSKKVAKKP TNKAPHPKQE |
| | | PQEINFPDDL PGSNTAAPVQ ETLHGCQPVT QEDGKESRIS VQERQ |
| 361 | OSM_MFE23_ | MGVLLTQRTL LSLVLALLFP SMASMQVQLQ QSGAELVRSG TSVKLSCTAS |
| | spCD28_ | GFNIKDSYMH WLRQGPEQGL EWIGWIDPEN GDTEYAPKFQ GKATFTTDTS |
| | CD28_CD40 | SNTAYLQLSS LTSEDTAVYY CNEGTPTGPY YFDYWGQGTT VTVSSGGGGS |
| | (SVQE-AVQA) | GGGGSGGGGS ENVLTQSPAI MSASPGEKVT ITCSASSSVS YMHWFQQKPG |
| | CTP195 | TSPKLWIYST SNLASGVPAR FSGSGSGTSY SLTISRMEAE DAATYYCQQR |
| | | SSYPLTFGAG TKLELKRAAA GSGGSGILVK QSPMLVAYDN AVNLSCKYSY |
| | | NLFSREFRAS LHKGLDSAVE VCVVYGNYSQ QLQVYSKTGF NCDGKLGNES |
| | | VTFYLQNLYV NQTDIYFCKI EVMYPPPYLD NEKSNGTIIH VKGKHLCPSP |
| | | LFPGPSKPFW VLVVVGGVLA CYSLLVTVAF IIFWVRSKRS RLLHSDYMNM |
| | | TPRRPGPTRK HYQPYAPPRD FAAYRSKKVA KKPTNKAPHP KQEPQEINFP |
| | | DDLPGSNTAA PVQETLHGCQ PVTQEDGKES RIAVQARQ |
| 362 | OSM_MFE23_ | MGVLLTQRTL LSLVLALLFP SMASMQVQLQ QSGAELVRSG TSVKLSCTAS |
| | spCD28_ | GFNIKDSYMH WLRQGPEQGL EWIGWIDPEN GDTEYAPKFQ GKATFTTDTS |
| | CD28_CD40 | SNTAYLQLSS LTSEDTAVYY CNEGTPTGPY YFDYWGQGTT VTVSSGGGGS |
| | (PVQET-AVAEA) | GGGGSGGGGS ENVLTQSPAI MSASPGEKVT ITCSASSSVS YMHWFQQKPG |
| | CTP196 | TSPKLWIYST SNLASGVPAR FSGSGSGTSY SLTISRMEAE DAATYYCQQR |
| | | SSYPLTFGAG TKLELKRAAA GSGGSGILVK QSPMLVAYDN AVNLSCKYSY |
| | | NLFSREFRAS LHKGLDSAVE VCVVYGNYSQ QLQVYSKTGF NCDGKLGNES |
| | | VTFYLQNLYV NQTDIYFCKI EVMYPPPYLD NEKSNGTIIH VKGKHLCPSP |
| | | LFPGPSKPFW VLVVVGGVLA CYSLLVTVAF IIFWVRSKRS RLLHSDYMNM |
| | | TPRRPGPTRK HYQPYAPPRD FAAYRSKKVA KKPTNKAPHP KQEPQEINFP |
| | | DDLPGSNTAA AVAEALHGCQ PVTQEDGKES RISVQERQ |
| 363 | OSM_MFE23_ | MGVLLTQRTL LSLVLALLFP SMASMQVQLQ QSGAELVRSG TSVKLSCTAS |
| | spCD28_ | GFNIKDSYMH WLRQGPEQGL EWIGWIDPEN GDTEYAPKFQ GKATFTTDTS |
| | CD28_CD40 | SNTAYLQLSS LTSEDTAVYY CNEGTPTGPY YFDYWGQGTT VTVSSGGGGS |
| | (PQEINF-AQAINF) | GGGGSGGGGS ENVLTQSPAI MSASPGEKVT ITCSASSSVS YMHWFQQKPG |
| | CTP197 | TSPKLWIYST SNLASGVPAR FSGSGSGTSY SLTISRMEAE DAATYYCQQR |
| | | SSYPLTFGAG TKLELKRAAA GSGGSGILVK QSPMLVAYDN AVNLSCKYSY |
| | | NLFSREFRAS LHKGLDSAVE VCVVYGNYSQ QLQVYSKTGF NCDGKLGNES |
| | | VTFYLQNLYV NQTDIYFCKI EVMYPPPYLD NEKSNGTIIH VKGKHLCPSP |
| | | LFPGPSKPFW VLVVVGGVLA CYSLLVTVAF IIFWVRSKRS RLLHSDYMNM |
| | | TPRRPGPTRK HYQPYAPPRD FAAYRSKKVA KKPTNKAPHP KQEAQAINFP |
| | | DDLPGSNTAA PVQETLHGCQ PVTQEDGKES RISVQERQ |
| 364 | OSM_MFE23_ | MGVLLTQRTL LSLVLALLFP SMASMQVQLQ QSGAELVRSG TSVKLSCTAS |
| | spCD28_ | GFNIKDSYMH WLRQGPEQGL EWIGWIDPEN GDTEYAPKFQ GKATFTTDTS |
| | CD28_CD40 | SNTAYLQLSS LTSEDTAVYY CNEGTPTGPY YFDYWGQGTT VTVSSGGGGS |
| | (P227A) | GGGGSGGGGS ENVLTQSPAI MSASPGEKVT ITCSASSSVS YMHWFQQKPG |
| | CTP198 | TSPKLWIYST SNLASGVPAR FSGSGSGTSY SLTISRMEAE DAATYYCQQR |
| | | SSYPLTFGAG TKLELKRAAA GSGGSGILVK QSPMLVAYDN AVNLSCKYSY |
| | | NLFSREFRAS LHKGLDSAVE VCVVYGNYSQ QLQVYSKTGF NCDGKLGNES |
| | | VTFYLQNLYV NQTDIYFCKI EVMYPPPYLD NEKSNGTIIH VKGKHLCPSP |
| | | LFPGPSKPFW VLVVVGGVLA CYSLLVTVAF IIFWVRSKRS RLLHSDYMNM |
| | | TPRRPGPTRK HYQPYAPPRD FAAYRSKKVA KKPTNKAAHP KQEPQEINFP |
| | | DDLPGSNTAA PVQETLHGCQ PVTQEDGKES RISVQERQ |
| 365 | OSM_MFE23_ | MGVLLTQRTL LSLVLALLFP SMASMQVOLQ QSGAELVRSG TSVKLSCTAS |
| | spCD28_CD28_ | GFNIKDSYMH WLRQGPEQGL EWIGWIDPEN GDTEYAPKFQ GKATFTTDTS |
| | CD40(Q263A) | SNTAYLQLSS LTSEDTAVYY CNEGTPTGPY YFDYWGQGTT VTVSSGGGGS |
| | CTP199 | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGILVK QSPMLVAYDN AVNLSCKYSY |
| | | NLFSREFRAS LHKGLDSAVE VCVVYGNYSQ QLQVYSKTGF NCDGKLGNES |
| | | VTFYLQNLYV NQTDIYFCKI EVMYPPPYLD NEKSNGTIIH VKGKHLCPSP |
| | | LFPGPSKPFW VLVVVGGVLA CYSLLVTVAF IIFWVRSKRS RLLHSDYMNM |
| | | TPRRPGPTRK HYQPYAPPRD FAAYRSKKVA KKPTNKAPHP KQEPQEINFP |
| | | DDLPGSNTAA PVQETLHGCQ PVTAEDGKES RISVQERQ |
| 366 | OSM_MFE23_ | MGVLLTQRTL LSLVLALLFP SMASMQVQLQ QSGAELVRSG TSVKLSCTAS |
| | spCD28_ | GFNIKDSYMH WLRQGPEQGL EWIGWIDPEN GDTEYAPKFQ GKATFTTDTS |
| | CD28_ | SNTAYLQLSS LTSEDTAVYY CNEGTPTGPY YFDYWGQGTT VTVSSGGGGS |
| | CD40_ | GGGGSGGGGS ENVLTQSPAI MSASPGEKVT ITCSASSSVS YMHWFQQKPG |
| | CD40_CD40 | TSPKLWIYST SNLASGVPAR FSGSGSGTSY SLTISRMEAE DAATYYCQQR |
| | CTP200 | SSYPLTFGAG TKLELKRAAA GSGGSGILVK QSPMLVAYDN AVNLSCKYSY |
| | | NLFSREFRAS LHKGLDSAVE VCVVYGNYSQ QLQVYSKTGF NCDGKLGNES |
| | | VTFYLQNLYV NQTDIYFCKI EVMYPPPYLD NEKSNGTIIH VKGKHLCPSP |
| | | LFPGPSKPFW VLVVVGGVLA CYSLLVTVAF IIFWVRSKRS RLLHSDYMNM |
| | | TPRRPGPTRK HYQPYAPPRD FAAYRSKKVA KKPTNKAPHP KQEPQEINFP |
| | | DDLPGSNTAA PVQETLHGCQ PVTAEDGKES RISVQERQKK VAKKPTNKAP |
| | | HPKQEPQEIN FPDDLPGSNT AAPVQETLHG CQPVTAEDGK ESRISVQERQ |
| 367 | OSM_MFE23_ | MGVLLTQRTL LSLVLALLFP SMASMQVQLQ QSGAELVRSG TSVKLSCTAS |
| | spCD28_CD28 | GFNIKDSYMH WLRQGPEQGL EWIGWIDPEN GDTEYAPKFQ GKATFTTDTS |
| | (PYAPP-AYAA)_ | SNTAYLQLSS LTSEDTAVYY CNEGTPTGPY YFDYWGQGTT VTVSSGGGGS |
| | CD40 | GGGGSGGGGS ENVLTQSPAI MSASPGEKVT ITCSASSSVS YMHWFQQKPG |
| | CTP201 | TSPKLWIYST SNLASGVPAR FSGSGSGTSY SLTISRMEAE DAATYYCQQR |
| | | SSYPLTFGAG TKLELKRAAA GSGGSGILVK QSPMLVAYDN AVNLSCKYSY |
| | | NLFSREFRAS LHKGLDSAVE VCVVYGNYSQ QLQVYSKTGF NCDGKLGNES |
| | | VTFYLQNLYV NQTDIYFCKI EVMYPPPYLD NEKSNGTIIH VKGKHLCPSP |
| | | LFPGPSKPFW VLVVVGGVLA CYSLLVTVAF IIFWVRSKRS RLLHSDYMNM |
| | | TPRRPGPTRK HYQAYAAPRD FAAYRSKKVA KKPTNKAPHP KQEPQEINFP |
| | | DDLPGSNTAA PVQETLHGCQ PVTQEDGKES RISVQERQ |
| 368 | OSM_MFE23_ | MGVLLTQRTL LSLVLALLFP SMASMQVOLQ QSGAELVRSG TSVKLSCTAS |
| | spCD28_CD28 | GFNIKDSYMH WLRQGPEQGL EWIGWIDPEN GDTEYAPKFQ GKATFTTDTS |
| | (YMNM-FMNM)_ | SNTAYLQLSS LTSEDTAVYY CNEGTPTGPY YFDYWGQGTT VTVSSGGGGS |
| | CD40 | GGGGSGGGGS ENVLTQSPAI MSASPGEKVT ITCSASSSVS YMHWFQQKPG |
| | CTP202 | TSPKLWIYST SNLASGVPAR FSGSGSGTSY SLTISRMEAE DAATYYCQQR |
| | | SSYPLTFGAG TKLELKRAAA GSGGSGILVK QSPMLVAYDN AVNLSCKYSY |
| | | NLFSREFRAS LHKGLDSAVE VCVVYGNYSQ QLQVYSKTGF NCDGKLGNES |
| | | VTFYLQNLYV NQTDIYFCKI EVMYPPPYLD NEKSNGTIIH VKGKHLCPSP |
| | | LFPGPSKPFW VLVVVGGVLA CYSLLVTVAF IIFWVRSKRS RLLHSDFMNM |
| | | TPRRPGPTRK HYQPYAPPRD FAAYRSKKVA KKPTNKAPHP KQEPQEINFP |
| | | DDLPGSNTAA PVQETLHGCQ PVTQEDGKES RISVQERQ |
| 369 | OSM_MFE23_ | MGVLLTQRTL LSLVLALLFP SMASMQVQLQ QSGAELVRSG TSVKLSCTAS |
| | spCD28_CD40 | GFNIKDSYMH WLRQGPEQGL EWIGWIDPEN GDTEYAPKFQ GKATFTTDTS |
| | | SNTAYLQLSS LTSEDTAVYY CNEGTPTGPY YFDYWGQGTT VTVSSGGGGS |
| | | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGILVK QSPMLVAYDN AVNLSCKYSY |
| | | NLFSREFRAS LHKGLDSAVE VCVVYGNYSQ QLQVYSKTGF NCDGKLGNES |
| | | VTFYLQNLYV NQTDIYFCKI EVMYPPPYLD NEKSNGTIIH VKGKHLCPSP |
| | | LFPGPSKPFW VLVVVGGVLA CYSLLVTVAF IIFWVKKVAK KPTNKAPHPK |
| | | QEPQEINFPD DLPGSNTAAP VQETLHGCQP VTQEDGKESR ISVQERQ |
| 370 | OSM_MFE23_ | MGVLLTQRTL LSLVLALLFP SMASMQVQLQ QSGAELVRSG TSVKLSCTAS |
| | spCD28_ | GFNIKDSYMH WLRQGPEQGL EWIGWIDPEN GDTEYAPKFQ GKATFTTDTS |
| | CD137_CD40 | SNTAYLQLSS LTSEDTAVYY CNEGTPTGPY YFDYWGQGTT VTVSSGGGGS |
| | | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGILVK QSPMLVAYDN AVNLSCKYSY |
| | | NLFSREFRAS LHKGLDSAVE VCVVYGNYSQ QLQVYSKTGF NCDGKLGNES |
| | | VTFYLQNLYV NQTDIYFCKI EVMYPPPYLD NEKSNGTIIH VKGKHLCPSP |
| | | LFPGPSKPFW VLVVVGGVLA CYSLLVTVAF IIFWVRFSVV KRGRKKLLYI |
| | | FKQPFMRPVQ TTQEEDGCSC RFPEEEEGGC EKKVAKKPTN KAPHPKQEPQ |
| | | EINFPDDLPG SNTAAPVQET LHGCQPVTQE DGKESRISVQ ERQ |
| 371 | OSM_MFE23_ | MGVLLTQRTL LSLVLALLFP SMASMQVQLQ QSGAELVRSG TSVKLSCTAS |
| | spCD28_ | GFNIKDSYMH WLRQGPEQGL EWIGWIDPEN GDTEYAPKFQ GKATFTTDTS |
| | CD134_CD40 | SNTAYLQLSS LTSEDTAVYY CNEGTPTGPY YFDYWGQGTT VTVSSGGGGS |
| | | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGILVK QSPMLVAYDN AVNLSCKYSY |
| | | NLFSREFRAS LHKGLDSAVE VCVVYGNYSQ QLQVYSKTGF NCDGKLGNES |
| | | VTFYLQNLYV NQTDIYFCKI EVMYPPPYLD NEKSNGTIIH VKGKHLCPSP |
| | | LFPGPSKPFW VLVVVGGVLA CYSLLVTVAF IIFWVRRDQR LPPDAHKPPG |
| | | GGSFRTPIQE EQADAHSTLA KIKKVAKKPT NKAPHPKQEP QEINFPDDLP |
| | | GSNTAAPVQE TLHGCQPVTQ EDGKESRISV QERQ |
| 372 | OSM_MFE23_ | MGVLLTQRTL LSLVLALLFP SMASMQVQLQ QSGAELVRSG TSVKLSCTAS |
| | spCD28_ | GFNIKDSYMH WLRQGPEQGL EWIGWIDPEN GDTEYAPKFQ GKATFTTDTS |
| | CD2_CD40 | SNTAYLQLSS LTSEDTAVYY CNEGTPTGPY YFDYWGQGTT VTVSSGGGGS |
| | | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGILVK QSPMLVAYDN AVNLSCKYSY |
| | | NLFSREFRAS LHKGLDSAVE VCVVYGNYSQ QLQVYSKTGF NCDGKLGNES |
| | | VTFYLQNLYV NQTDIYFCKI EVMYPPPYLD NEKSNGTIIH VKGKHLCPSP |
| | | LFPGPSKPFW VLVVVGGVLA CYSLLVTVAF IIFWVKRKKQ RSRRNDEELE |
| | | TRAHRVATEE RGRKPHQIPA STPQNPATSQ HPPPPPGHRS QAPSHRPPPP |
| | | GHRVQHQPQK RPPAPSGTQV HQQKGPPLPR PRVQPKPPHG AAENSLSPSS |
| | | NKKVAKKPTN KAPHPKQEPQ EINFPDDLPG SNTAAPVQET LHGCQPVTQE |
| | | DGKESRISVQ ERQ |
| 373 | OSM_MFE23_ | MGVLLTQRTL LSLVLALLFP SMASMQVQLQ QSGAELVRSG TSVKLSCTAS |
| | spCD28_ | GFNIKDSYMH WLRQGPEQGL EWIGWIDPEN GDTEYAPKFQ GKATFTTDTS |
| | GITR_CD40 | SNTAYLQLSS LTSEDTAVYY CNEGTPTGPY YFDYWGQGTT VTVSSGGGGS |
| | | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGILVK QSPMLVAYDN AVNLSCKYSY |
| | | NLFSREFRAS LHKGLDSAVE VCVVYGNYSQ QLQVYSKTGF NCDGKLGNES |
| | | VTFYLQNLYV NQTDIYFCKI EVMYPPPYLD NEKSNGTIIH VKGKHLCPSP |
| | | LFPGPSKPFW VLVVVGGVLA CYSLLVTVAF IIFWVQLGLH IWQLRSQCMW |
| | | PRETQLLLEV PPSTEDARSC QFPEEERGER SAEEKGRLGD LWVKKVAKKP |
| | | TNKAPHPKQE PQEINFPDDL PGSNTAAPVQ ETLHGCQPVT QEDGKESRIS |
| | | VQERQ |
| 374 | OSM_MFE23_ | MGVLLTQRTL LSLVLALLFP SMASMQVQLQ QSGAELVRSG TSVKLSCTAS |
| | spCD28_ | GFNIKDSYMH WLRQGPEQGL EWIGWIDPEN GDTEYAPKFQ GKATFTTDTS |
| | CD29_CD40 | SNTAYLQLSS LTSEDTAVYY CNEGTPTGPY YFDYWGQGTT VTVSSGGGGS |
| | | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGILVK QSPMLVAYDN AVNLSCKYSY |
| | | NLFSREFRAS LHKGLDSAVE VCVVYGNYSQ QLQVYSKTGF NCDGKLGNES |
| | | VTFYLQNLYV NQTDIYFCKI EVMYPPPYLD NEKSNGTIIH VKGKHLCPSP |
| | | LFPGPSKPFW VLVVVGGVLA CYSLLVTVAF IIFWVKLLMI IHDRREFAKF |
| | | EKEKMNAKWD TGENPIYKSA VTTVVNPKYE GKKKVAKKPT NKAPHPKQEP |
| | | QEINFPDDLP GSNTAAPVQE TLHGCQPVTQ EDGKESRISV QERQ |
| 375 | OSM_MFE23_ | MGVLLTQRTL LSLVLALLFP SMASMQVQLQ QSGAELVRSG TSVKLSCTAS |
| | spCD28_ | GFNIKDSYMH WLRQGPEQGL EWIGWIDPEN GDTEYAPKFQ GKATFTTDTS |
| | CD150_CD40 | SNTAYLQLSS LTSEDTAVYY CNEGTPTGPY YFDYWGQGTT VTVSSGGGGS |
| | | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGILVK QSPMLVAYDN AVNLSCKYSY |
| | | NLFSREFRAS LHKGLDSAVE VCVVYGNYSQ QLQVYSKTGF NCDGKLGNES |
| | | VTFYLQNLYV NQTDIYFCKI EVMYPPPYLD NEKSNGTIIH VKGKHLCPSP |
| | | LFPGPSKPFW VLVVVGGVLA CYSLLVTVAF IIFWVRRRGK TNHYQTTVEK |
| | | KSLTIYAQVQ KPGPLQKKLD SFPAQDPCTT IYVAATEPVP ESVQETNSIT |
| | | VYASVTLPES KKVAKKPTNK APHPKQEPQE INFPDDLPGS NTAAPVQETL |
| | | HGCQPVTQED GKESRISVQE RQ |
| 376 | OSM_MFE23_ | MGVLLTQRTL LSLVLALLFP SMASMQVQLQ QSGAELVRSG TSVKLSCTAS |
| | spCD8_CD40 | GFNIKDSYMH WLRQGPEQGL EWIGWIDPEN GDTEYAPKFQ GKATFTTDTS |
| | CTP193 | SNTAYLQLSS LTSEDTAVYY CNEGTPTGPY YFDYWGQGTT VTVSSGGGGS |
| | | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGFVPV FLPAKPTTTP APRPPTPAPT |
| | | IASQPLSLRP EACRPAAGGA VHTRGLDFAC DIYIWAPLAG TCGVLLLSLV |
| | | ITLYCNHRNK KVAKKPTNKA PHPKQEPQEI NFPDDLPGSN TAAPVQETLH |
| | | GCQPVTQEDG KESRISVQER Q |
| 377 | OSM_MFE23_ | MGVLLTQRTL LSLVLALLFP SMASMQVQLQ QSGAELVRSG TSVKLSCTAS |
| | spCD8_ | GFNIKDSYMH WLRQGPEQGL EWIGWIDPEN GDTEYAPKFQ GKATFTTDTS |
| | CD137_CD40 | SNTAYLQLSS LTSEDTAVYY CNEGTPTGPY YFDYWGQGTT VTVSSGGGGS |
| | CTP192 | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGFVPV FLPAKPTTTP APRPPTPAPT |
| | | IASQPLSLRP EACRPAAGGA VHTRGLDFAC DIYIWAPLAG TCGVLLLSLV |
| | | ITLYCNHRNR FSVVKRGRKK LLYIFKQPFM RPVQTTQEED GCSCRFPEEE |
| | | EGGGEKKVAK KPTNKAPHPK QEPQEINFPD DLPGSNTAAP VQETLHGCQP |
| | | VTQEDGKESR ISVQERQ |
| 378 | OSM_MFE23_ | MGVLLTQRTL LSLVLALLFP SMASMQVOLQ QSGAELVRSG TSVKLSCTAS |
| | spCD8_ | GFNIKDSYMH WLRQGPEQGL EWIGWIDPEN GDTEYAPKFQ GKATFTTDTS |
| | CD134_CD40 | SNTAYLQLSS LTSEDTAVYY CNEGTPTGPY YFDYWGQGTT VTVSSGGGGS |
| | | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGFVPV FLPAKPTTTP APRPPTPAPT |
| | | IASQPLSLRP EACRPAAGGA VHTRGLDFAC DIYIWAPLAG TCGVLLLSLV |
| | | ITLYCNHRNR RDQRLPPDAH KPPGGGSFRT PIQEEQADAH STLAKIKKVA |
| | | KKPTNKAPHP KQEPQEINFP DDLPGSNTAA PVQETLHGCQ PVTQEDGKSR |
| | | ISVQERQ |
| 379 | OSM_MFE23_ | MGVLLTQRTL LSLVLALLFP SMASMQVQLQ QSGAELVRSG TSVKLSCTAS |
| | spCD8_ | GFNIKDSYMH WLRQGPEQGL EWIGWIDPEN GDTEYAPKFQ GKATFTTDTS |
| | CD2_CD40 | SNTAYLQLSS LTSEDTAVYY CNEGTPTGPY YFDYWGQGTT VTVSSGGGGS |
| | CTP191 | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGFVPV FLPAKPTTTP APRPPTPAPT |
| | | IASQPLSLRP EACRPAAGGA VHTRGLDFAC DIYIWAPLAG TCGVLLLSLV |
| | | ITLYCNHRNK RKKQRSRRND EELETRAHRV ATEERGRKPH QIPASTPQNP |
| | | ATSQHPPPPP GHRSQAPSHR PPPPGHRVQH QPQKRPPAPS GTQVHQQKGP |
| | | PLPRPRVQPK PPHGAAENSL SPSSNKKVAK KPTNKAPHPK QEPQEINFPD |
| | | DLPGSNTAAP VQETLHGCQP VTQEDGKESR ISVQERQ |
| 380 | OSM_MFE23_ | MGVLLTQRTL LSLVLALLFP SMASMQVQLQ QSGAELVRSG TSVKLSCTAS |
| | spCD8_ | GFNIKDSYMH WLRQGPEQGL EWIGWIDPEN GDTEYAPKFQ GKATFTTDTS |
| | GITR_CD40 | SNTAYLQLSS LTSEDTAVYY CNEGTPTGPY YFDYWGQGTT VTVSSGGGGS |
| | | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGFVPV FLPAKPTTTP APRPPTPAPT |
| | | IASQPLSLRP EACRPAAGGA VHTRGLDFAC DIYIWAPLAG TCGVLLLSLV |
| | | ITLYCNHRNQ LGLHIWQLRS QCMWPRETQL LLEVPPSTED ARSCQFPEEE |
| | | RGERSAEEKG RLGDLWVKKV AKKPTNKAPH PKQEPQEINF PDDLPGSNTA |
| | | APVQETLHGC QPVTQEDGKE SRISVQERQ |
| 381 | OSM_MFE23_ | MGVLLTQRTL LSLVLALLFP SMASMQVQLQ QSGAELVRSG TSVKLSCTAS |
| | spCD8_ | GFNIKDSYMH WLRQGPEQGL EWIGWIDPEN GDTEYAPKFQ GKATFTTDTS |
| | CD29_CD40 | SNTAYLQLSS LTSEDTAVYY CNEGTPTGPY YFDYWGQGTT VTVSSGGGGS |
| | | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGFVPV FLPAKPTTTP APRPPTPAPT |
| | | IASQPLSLRP EACRPAAGGA VHTRGLDFAC DIYIWAPLAG TCGVLLLSLV |
| | | ITLYCNHRNK LLMIIHDRRE FAKFEKEKMN AKWDTGENPI YKSAVTTVVN |
| | | PKYEGKKKVA KKPTNKAPHP KQEPQEINFP DDLPGSNTAA PVQETLHGCQ |
| | | PVTQEDGKES RISVQERQ |
| 382 | OSM_MFE23_ | MGVLLTQRTL LSLVLALLFP SMASMQVQLQ QSGAELVRSG TSVKLSCTAS |
| | spCD8_ | GFNIKDSYMH WLRQGPEQGL EWIGWIDPEN GDTEYAPKFQ GKATFTTDTS |
| | CD150_CD40 | SNTAYLQLSS LTSEDTAVYY CNEGTPTGPY YFDYWGQGTT VTVSSGGGGS |
| | | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGFVPV FLPAKPTTTP APRPPTPAPT |
| | | IASQPLSLRP EACRPAAGGA VHTRGLDFAC DIYIWAPLAG TCGVLLLSLV |
| | | ITLYCNHRNR RRGKTNHYQT TVEKKSLTIY AQVQKPGPLQ KKLDSFPAQD |
| | | PCTTIYVAAT EPVPESVQET NSITVYASVT LPESKKVAKK PTNKAPHPKQ |
| | | EPQEINFPDD LPGSNTAAPV QETLHGCQPV TQEDGKESRI SVQERQ |
| 383 | OSM_MFE23_ | MGVLLTQRTL LSLVLALLFP SMASMQVQLQ QSGAELVRSG TSVKLSCTAS |
| | spIG4_ | GFNIKDSYMH WLRQGPEQGL EWIGWIDPEN GDTEYAPKFQ GKATFTTDTS |
| | CD28_CD40 | SNTAYLQLSS LTSEDTAVYY CNEGTPTGPY YFDYWGQGTT VTVSSGGGGS |
| | CTP203 | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGESKY GPPCPSCPAP EFLGGPSVFL |
| | | FPPKPKDTLM ISRTPEVTCV VVDVSQEDPE VQFNWYVDGV EVHNAKTKPR |
| | | EEQFNSTYRV VSVLTVLHQD WLNGKEYKCK VSNKGLPSSI EKTISKAKGQ |
| | | PREPQVYTLP PSQEEMTKNQ VSLTCLVKGF YPSDIAVEWE SNGQPENNYK |
| | | TTPPVLDSDG SFFLYSRLTV DKSRWQEGNV FSCSVMHEAL HNHYTQKSLS |
| | | LSLGKMFWVL VVVGGVLACY SLLVTVAFII FWVRSKRSRL LHSDYMNMTP |
| | | RRPGPTRKHY QPYAPPRDFA AYRSKKVAKK PTNKAPHPKQ EPQEINFPDD |
| | | LPGSNTAAPV QETLHGCQPV TQEDGKESRI SVQERQ |
| 384 | OSM_MFE23_ | MGVLLTQRTL LSLVLALLFP SMASMQVQLQ QSGAELVRSG TSVKLSCTAS |
| | spIG4_CD40 | GFNIKDSYMH WLRQGPEQGL EWIGWIDPEN GDTEYAPKFQ GKATFTTDTS |
| | | SNTAYLQLSS LTSEDTAVYY CNEGTPTGPY YFDYWGQGTT VTVSSGGGGS |
| | | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGESKY GPPCPSCPAP EFLGGPSVFL |
| | | FPPKPKDTLM ISRTPEVTCV VVDVSQEDPE VQFNWYVDGV EVHNAKTKPR |
| | | EEQFNSTYRV VSVLTVLHQD WLNGKEYKCK VSNKGLPSSI EKTISKAKGQ |
| | | PREPQVYTLP PSQEEMTKNQ VSLTCLVKGF YPSDIAVEWE SNGQPENNYK |
| | | TTPPVLDSDG SFFLYSRLTV DKSRWQEGNV FSCSVMHEAL HNHYTQKSLS |
| | | LSLGKMFWVL VVVGGVLACY SLLVTVAFII FWVKKVAKKP TNKAPHPKQE |
| | | PQEINFPDDL PGSNTAAPVQ ETLHGCQPVT QEDGKESRIS VQERQ |
| 385 | OSM_MFE23_ | MGVLLTQRTL LSLVLALLFP SMASMQVOLQ QSGAELVRSG TSVKLSCTAS |
| | spIG4_ | GFNIKDSYMH WLRQGPEQGL EWIGWIDPEN GDTEYAPKFQ GKATFTTDTS |
| | CD137_CD40 | SNTAYLQLSS LTSEDTAVYY CNEGTPTGPY YFDYWGQGTT VTVSSGGGGS |
| | | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGESKY GPPCPSCPAP EFLGGPSVFL |
| | | FPPKPKDTLM ISRTPEVTCV VVDVSQEDPE VQFNWYVDGV EVHNAKTKPR |
| | | EEQFNSTYRV VSVLTVLHQD WLNGKEYKCK VSNKGLPSSI EKTISKAKGQ |
| | | PREPQVYTLP PSQEEMTKNQ VSLTCLVKGF YPSDIAVEWE SNGQPENNYK |
| | | TTPPVLDSDG SFFLYSRLTV DKSRWQEGNV FSCSVMHEAL HNHYTQKSLS |
| | | LSLGKMFWVL VVVGGVLACY SLLVTVAFII FWVKKVAKKP TNKAPHPKQE |
| | | PQEINFPDDL PGSNTAAPVQ ETLHGCQPVT QEDGKESRIS VQERQ |
| 386 | OSM_MFE23_ | MGVLLTQRTL LSLVLALLFP SMASMQVQLQ QSGAELVRSG TSVKLSCTAS |
| | spIG4_ | GFNIKDSYMH WLRQGPEQGL EWIGWIDPEN GDTEYAPKFQ GKATFTTDTS |
| | CD134_CD40 | SNTAYLQLSS LTSEDTAVYY CNEGTPTGPY YFDYWGQGTT VTVSSGGGGS |
| | | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGESKY GPPCPSCPAP EFLGGPSVFL |
| | | FPPKPKDTLM ISRTPEVTCV VVDVSQEDPE VQFNWYVDGV EVHNAKTKPR |
| | | EEQFNSTYRV VSVLTVLHQD WLNGKEYKCK VSNKGLPSSI EKTISKAKGQ |
| | | PREPQVYTLP PSQEEMTKNQ VSLTCLVKGF YPSDIAVEWE SNGQPENNYK |
| | | TTPPVLDSDG SFFLYSRLTV DKSRWQEGNV FSCSVMHEAL HNHYTQKSLS |
| | | LSLGKMFWVL VVVGGVLACY SLLVTVAFII FWVRRDQRLP PDAHKPPGGG |
| | | SFRTPIQEEQ ADAHSTLAKI KKVAKKPTNK APHPKQEPQE INFPDDLPGS |
| | | NTAAPVQETL HGCQPVTQED GKESRISVQE RQ |
| 387 | OSM_MFE23_ | MGVLLTQRTL LSLVLALLFP SMASMQVQLQ QSGAELVRSG TSVKLSCTAS |
| | spIG4_ | GFNIKDSYMH WLRQGPEQGL EWIGWIDPEN GDTEYAPKFQ GKATFTTDTS |
| | CD2_CD40 | SNTAYLQLSS LTSEDTAVYY CNEGTPTGPY YFDYWGQGTT VTVSSGGGGS |
| | | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGESKY GPPCPSCPAP EFLGGPSVFL |
| | | FPPKPKDTLM ISRTPEVTCV VVDVSQEDPE VQFNWYVDGV EVHNAKTKPR |
| | | EEQFNSTYRV VSVLTVLHQD WLNGKEYKCK VSNKGLPSSI EKTISKAKGQ |
| | | PREPQVYTLP PSQEEMTKNQ VSLTCLVKGF YPSDIAVEWE SNGQPENNYK |
| | | TTPPVLDSDG SFFLYSRLTV DKSRWQEGNV FSCSVMHEAL HNHYTQKSLS |
| | | LSLGKMFWVL VVVGGVLACY SLLVTVAFII FWVKRKKQRS RRNDEELETR |
| | | AHRVATEERG RKPHQIPAST PQNPATSQHP PPPPGHRSQA PSHRPPPPGH |
| | | RVQHQPQKRP PAPSGTQVHQ QKGPPLPRPR VQPKPPHGAA ENSLSPSSNK |
| | | KVAKKPTNKA PHPKQEPQEI NFPDDLPGSN TAAPVQETLH GCQPVTQEDG |
| | | KESRISVQER Q |
| 388 | OSM_MFE23_ | MGVLLTQRTL LSLVLALLFP SMASMQVOLQ QSGAELVRSG TSVKLSCTAS |
| | spIG4_ | GFNIKDSYMH WLRQGPEQGL EWIGWIDPEN GDTEYAPKFQ GKATFTTDTS |
| | GITR_CD40 | SNTAYLQLSS LTSEDTAVYY CNEGTPTGPY YFDYWGQGTT VTVSSGGGGS |
| | | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGESKY GPPCPSCPAP EFLGGPSVFL |
| | | FPPKPKDTLM ISRTPEVTCV VVDVSQEDPE VQFNWYVDGV EVHNAKTKPR |
| | | EEQFNSTYRV VSVLTVLHQD WLNGKEYKCK VSNKGLPSSI EKTISKAKGQ |
| | | PREPQVYTLP PSQEEMTKNQ VSLTCLVKGF YPSDIAVEWE SNGQPENNYK |
| | | TTPPVLDSDG SFFLYSRLTV DKSRWQEGNV FSCSVMHEAL HNHYTQKSLS |
| | | LSLGKMFWVL VVVGGVLACY SLLVTVAFII FWVQLGLHIW QLRSQCMWPR |
| | | ETQLLLEVPP STEDARSCQF PEEERGERSA EEKGRLGDLW VKKVAKKPTN |
| | | KAPHPKQEPQ EINFPDDLPG SNTAAPVQET LHGCQPVTQE DGKESRISVQ |
| | | ERQ |
| 389 | OSM_MFE23_ | MGVLLTQRTL LSLVLALLFP SMASMQVQLQ QSGAELVRSG TSVKLSCTAS |
| | spIG4_ | GFNIKDSYMH WLRQGPEQGL EWIGWIDPEN GDTEYAPKFQ GKATFTTDTS |
| | CD29_CD40 | SNTAYLQLSS LTSEDTAVYY CNEGTPTGPY YFDYWGQGTT VTVSSGGGGS |
| | | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGESKY GPPCPSCPAP EFLGGPSVFL |
| | | FPPKPKDTLM ISRTPEVTCV VVDVSQEDPE VQFNWYVDGV EVHNAKTKPR |
| | | EEQFNSTYRV VSVLTVLHQD WLNGKEYKCK VSNKGLPSSI EKTISKAKGQ |
| | | PREPQVYTLP PSQEEMTKNQ VSLTCLVKGF YPSDIAVEWE SNGQPENNYK |
| | | TTPPVLDSDG SFFLYSRLTV DKSRWQEGNV FSCSVMHEAL HNHYTQKSLS |
| | | LSLGKMFWVL VVVGGVLACY SLLVTVAFII FWVKLLMIIH DRREFAKFEK |
| | | EKMNAKWDTG ENPIYKSAVT TVVNPKYEGK KKVAKKPTNK APHPKQEPQE |
| | | INFPDDLPGS NTAAPVQETL HGCQPVTQED GKESRISVQE RQ |
| 390 | OSM_MFE23_ | MGVLLTQRTL LSLVLALLFP SMASMQVQLQ QSGAELVRSG TSVKLSCTAS |
| | spIG4_ | GFNIKDSYMH WLRQGPEQGL EWIGWIDPEN GDTEYAPKFQ GKATFTTDTS |
| | CD150_CD40 | SNTAYLQLSS LTSEDTAVYY CNEGTPTGPY YFDYWGQGTT VTVSSGGGGS |
| | | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGESKY GPPCPSCPAP EFLGGPSVFL |
| | | FPPKPKDTLM ISRTPEVTCV VVDVSQEDPE VQFNWYVDGV EVHNAKTKPR |
| | | EEQFNSTYRV VSVLTVLHQD WLNGKEYKCK VSNKGLPSSI EKTISKAKGQ |
| | | PREPQVYTLP PSQEEMTKNQ VSLTCLVKGF YPSDIAVEWE SNGQPENNYK |
| | | TTPPVLDSDG SFFLYSRLTV DKSRWQEGNV FSCSVMHEAL HNHYTQKSLS |
| | | LSLGKMFWVL VVVGGVLACY SLLVTVAFII FWVRRRGKTN HYQTTVEKKS |
| | | LTIYAQVQKP GPLQKKLDSF PAQDPCTTIY VAATEPVPES VQETNSITVY |
| | | ASVTLPESKK VAKKPTNKAP HPKQEPQEIN FPDDLPGSNT AAPVQETLHG |
| | | CQPVTQEDGK ESRISVQERQ |
| 391 | OSM_MFE23_ | MGVLLTQRTL LSLVLALLFP SMASMQVQLQ QSGAELVRSG TSVKLSCTAS |
| | spIG4_ | GFNIKDSYMH WLRQGPEQGL EWIGWIDPEN GDTEYAPKFQ GKATFTTDTS |
| | CD40_tandem | SNTAYLQLSS LTSEDTAVYY CNEGTPTGPY YFDYWGQGTT VTVSSGGGGS |
| | | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGESKY GPPCPSCPAP EFLGGPSVFL |
| | | FPPKPKDTLM ISRTPEVTCV VVDVSQEDPE VQFNWYVDGV EVHNAKTKPR |
| | | EEQFNSTYRV VSVLTVLHQD WLNGKEYKCK VSNKGLPSSI EKTISKAKGQ |
| | | PREPQVYTLP PSQEEMTKNQ VSLTCLVKGF YPSDIAVEWE SNGQPENNYK |
| | | TTPPVLDSDG SFFLYSRLTV DKSRWQEGNV FSCSVMHEAL HNHYTQKSLS |
| | | LSLGKMFWVL VVVGGVLACY SLLVTVAFII FWVAKKPTNK APHPKQEPQE |
| | | INFPDDLPGS NTAAPVQETL HGCQPVTQED GKESRISVQE RQKKVAKKPT |
| | | NKAPHPKQEP QEINFPDDLP GSNTAAPVQE TLHGCQPVTQ EDGKESRISV |
| | | QERQKKVA |
| 392 | OSM_MFE23_ | MGVLLTQRTL LSLVLALLFP SMASMQVQLQ QSGAELVRSG TSVKLSCTAS |
| | spIG4_ | GFNIKDSYMH WLRQGPEQGL EWIGWIDPEN GDTEYAPKFQ GKATFTTDTS |
| | CD40_P227A | SNTAYLQLSS LTSEDTAVYY CNEGTPTGPY YFDYWGQGTT VTVSSGGGGS |
| | | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGESKY GPPCPSCPAP EFLGGPSVFL |
| | | FPPKPKDTLM ISRTPEVTCV VVDVSQEDPE VQFNWYVDGV EVHNAKTKPR |
| | | EEQFNSTYRV VSVLTVLHQD WLNGKEYKCK VSNKGLPSSI EKTISKAKGQ |
| | | PREPQVYTLP PSQEEMTKNQ VSLTCLVKGF YPSDIAVEWE SNGQPENNYK |
| | | TTPPVLDSDG SFFLYSRLTV DKSRWQEGNV FSCSVMHEAL HNHYTQKSLS |
| | | LSLGKMFWVL VVVGGVLACY SLLVTVAFII FWVKKVAKKP TNKAAHPKQE |
| | | PQEINFPDDL PGSNTAAPVQ ETLHGCQPVT QEDGKESRIS VQERQ |
| 393 | OSM_MFE23_PD1_ | MGVLLTQRTL LSLVLALLFP SMASMQVOLQ QSGAELVRSG TSVKLSCTAS |
| | sCD28TM_CD28_ | GFNIKDSYMH WLRQGPEQGL EWIGWIDPEN GDTEYAPKFQ GKATFTTDTS |
| | CD40 | SNTAYLQLSS LTSEDTAVYY CNEGTPTGPY YFDYWGQGTT VTVSSGGGGS |
| | CTP204 | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGRPGW FLDSPDRPWN PPTFSPALLV |
| | | VTEGDNATFT CSFSNTSESF VLNWYRMSPS NQTDKLAAFP EDRSQPGQDC |
| | | RFRVTQLPNG RDFHMSVVRA RRNDSGTYLC GAISLAPKAQ IKESLRAELR |
| | | VTERRAEVPT AHCPSPLFPG PSKPFWVLVV VGGVLACYSL LVTVAFIIFW |
| | | VRSKRSRLLH SDYMNMTPRR PGPTRKHYQP YAPPRDFAAY RSKKVAKKPT |
| | | NKAPHPKQEP QEINFPDDLP GSNTAAPVQE TLHGCQPVTQ EDGKESRISV |
| | | QERQ |
| 394 | OSM_MFE23_PD1_ | MGVLLTQRTL LSLVLALLFP SMASMQVQLQ QSGAELVRSG TSVKLSCTAS |
| | sCD28TM_CD40 | GFNIKDSYMH WLRQGPEQGL EWIGWIDPEN GDTEYAPKFQ GKATFTTDTS |
| | | SNTAYLQLSS LTSEDTAVYY CNEGTPTGPY YFDYWGQGTT VTVSSGGGGS |
| | | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGRPGW FLDSPDRPWN PPTFSPALLV |
| | | VTEGDNATFT CSFSNTSESF VLNWYRMSPS NQTDKLAAFP EDRSQPGQDC |
| | | RFRVTQLPNG RDFHMSVVRA RRNDSGTYLC GAISLAPKAQ IKESLRAELR |
| | | VTERRAEVPT AHCPSPLFPG PSKPFWVLVV VGGVLACYSL LVTVAFIIFW |
| | | VKKVAKKPTN KAPHPKQEPQ EINFPDDLPG SNTAAPVQET LHGCQPVTQE |
| | | DGKESRISVQ ERQ |
| 395 | OSM_MFE23_ | MGVLLTQRTL LSLVLALLFP SMASMQVQLQ QSGAELVRSG TSVKLSCTAS |
| | TIGIT_sCD28TM_ | GFNIKDSYMH WLRQGPEQGL EWIGWIDPEN GDTEYAPKFQ GKATFTTDTS |
| | CD28_CD40 | SNTAYLQLSS LTSEDTAVYY CNEGTPTGPY YFDYWGQGTT VTVSSGGGGS |
| | | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGMMTG TIETTGNISA EKGGSIILQC |
| | | HLSSTTAQVT QVNWEQQDQL LAICNADLGW HISPSFKDRV APGPGLGLTL |
| | | QSLTVNDTGE YFCIYHTYPD GTYTGRIFLE VLESSVAEHG ARFQIPFWVL |
| | | VVVGGVLACY SLLVTVAFII FWVRSKRSRL LHSDYMNMTP RRPGPTRKHY |
| | | QPYAPPRDFA AYRSKKVAKK PTNKAPHPKQ EPQEINFPDD LPGSNTAAPV |
| | | QETLHGCQPV TQEDGKESRI SVQERQ |
| 396 | OSM_MFE23_ | MGVLLTQRTL LSLVLALLFP SMASMQVQLQ QSGAELVRSG TSVKLSCTAS |
| | TIGIT_sCD28TM_ | GFNIKDSYMH WLRQGPEQGL EWIGWIDPEN GDTEYAPKFQ GKATFTTDTS |
| | CD40 | SNTAYLQLSS LTSEDTAVYY CNEGTPTGPY YFDYWGQGTT VTVSSGGGGS |
| | | GGGGSGGGGS ENVLTQSPAI MSASPGEKVT ITCSASSSVS YMHWFQQKPG |
| | | TSPKLWIYST SNLASGVPAR FSGSGSGTSY SLTISRMEAE DAATYYCQQR |
| | | SSYPLTFGAG TKLELKRAAA GSGGSGMMTG TIETTGNISA EKGGSIILQC |
| | | HLSSTTAQVT QVNWEQQDQL LAICNADLGW HISPSFKDRV APGPGLGLTL |
| | | QSLTVNDTGE YFCIYHTYPD GTYTGRIFLE VLESSVAEHG ARFQIPFWVL |
| | | VVVGGVLACY SLLVTVAFII FWVKKVAKKP TNKAPHPKQE PQEINFPDDL |
| | | PGSNTAAPVQ ETLHGCQPVT QEDGKESRIS VQERQ |
| 397 | OSM_MOV19_ | MGVLLTQRTL LSLVLALLFP SMASMQVQLQ QSGAELVKPG ASVKISCKAS |
| | spCD28_ | GYSFTGYFMN WVKQSHGKSL EWIGRIHPYD GDTFYNQNFK DKATLTVDKS |
| | CD28_CD40 | SNTAHMELLS LTSEDFAVYY CTRYDGSRAM DYWGQGTTVT VSSGGGGSGG |
| | | GGSGGGGSDI ELTQSPASLA VSLGQRAIIS CKASQSVSFA GTSLMHWYHQ |
| | | KPGQQPKLLI YRASNLEAGV PTRFSGSGSK TDFTLNIHPV EEEDAATYYC |
| | | QQSREYPYTF GGGTKLEIKA AAGSGGSGIL VKQSPMLVAY DNAVNLSCKY |
| | | SYNLFSREFR ASLHKGLDSA VEVCVVYGNY SQQLQVYSKT GFNCDGKLGN |
| | | ESVTFYLQNL YVNQTDIYFC KIEVMYPPPY LDNEKSNGTI IHVKGKHLCP |
| | | SPLFPGPSKP FWVLVVVGGV LACYSLLVTV AFIIFWVRSK RSRLLHSDYM |
| | | NMTPRRPGPT RKHYQPYAPP RDFAAYRSKK VAKKPTNKAP HPKQEPQEIN |
| | | FPDDLPGSNT AAPVQETLHG CQPVTQEDGK ESRISVQERQ |
| 398 | OSM_MOV19_ | MGVLLTQRTL LSLVLALLFP SMASMQVQLQ QSGAELVKPG ASVKISCKAS |
| | spCD28_CD40 | GYSFTGYFMN WVKQSHGKSL EWIGRIHPYD GDTFYNQNFK DKATLTVDKS |
| | | SNTAHMELLS LTSEDFAVYY CTRYDGSRAM DYWGQGTTVT VSSGGGGSGG |
| | | GGSGGGGSDI ELTQSPASLA VSLGQRAIIS CKASQSVSFA GTSLMHWYHQ |
| | | KPGQQPKLLI YRASNLEAGV PTRFSGSGSK TDFTLNIHPV EEEDAATYYC |
| | | QQSREYPYTF GGGTKLEIKA AAGSGGSGIL VKQSPMLVAY DNAVNLSCKY |
| | | SYNLFSREFR ASLHKGLDSA VEVCVVYGNY SQQLQVYSKT GFNCDGKLGN |
| | | ESVTFYLQNL YVNQTDIYFC KIEVMYPPPY LDNEKSNGTI IHVKGKHLCP |
| | | SPLFPGPSKP FWVLVVVGGV LACYSLLVTV AFIIFWVKKV AKKPTNKAPH |
| | | PKQEPQEINF PDDLPGSNTA APVQETLHGC QPVTQEDGKE SRISVQERQ |
| 399 | OSM_MOV19_ | MGVLLTQRTL LSLVLALLFP SMASMQVQLQ QSGAELVKPG ASVKISCKAS |
| | spCD28_ | GYSFTGYFMN WVKQSHGKSL EWIGRIHPYD GDTFYNQNFK DKATLTVDKS |
| | CD137_CD40 | SNTAHMELLS LTSEDFAVYY CTRYDGSRAM DYWGQGTTVT VSSGGGGSGG |
| | | GGSGGGGSDI ELTQSPASLA VSLGQRAIIS CKASQSVSFA GTSLMHWYHQ |
| | | KPGQQPKLLI YRASNLEAGV PTRFSGSGSK TDFTLNIHPV EEEDAATYYC |
| | | QQSREYPYTF GGGTKLEIKA AAGSGGSGIL VKQSPMLVAY DNAVNLSCKY |
| | | SYNLFSREFR ASLHKGLDSA VEVCVVYGNY SQQLQVYSKT GFNCDGKLGN |
| | | ESVTFYLQNL YVNQTDIYFC KIEVMYPPPY LDNEKSNGTI IHVKGKHLCP |
| | | SPLFPGPSKP FWVLVVVGGV LACYSLLVTV AFIIFWVRFS WKRGRKKLL |
| | | YIFKQPFMRP VQTTQEEDGC SCRFPEEEEG GCEKKVAKKP TNKAPHPKQE |
| | | PQEINFPDDL PGSNTAAPVQ ETLHGCQPVT QEDGKESRIS VQERQ |
| 400 | OSM_MOV19_ | MGVLLTQRTL LSLVLALLFP SMASMQVOLQ QSGAELVKPG ASVKISCKAS |
| | spCD28_ | GYSFTGYFMN WVKQSHGKSL EWIGRIHPYD GDTFYNQNFK DKATLTVDKS |
| | CD134_CD40 | SNTAHMELLS LTSEDFAVYY CTRYDGSRAM DYWGQGTTVT VSSGGGGSGG |
| | | GGSGGGGSDI ELTQSPASLA VSLGQRAIIS CKASQSVSFA GTSLMHWYHQ |
| | | KPGQQPKLLI YRASNLEAGV PTRFSGSGSK TDFTLNIHPV EEEDAATYYC |
| | | QQSREYPYTF GGGTKLEIKA AAGSGGSGIL VKQSPMLVAY DNAVNLSCKY |
| | | SYNLFSREFR ASLHKGLDSA VEVCVVYGNY SQQLQVYSKT GFNCDGKLGN |
| | | ESVTFYLQNL YVNQTDIYFC KIEVMYPPPY LDNEKSNGTI IHVKGKHLCP |
| | | SPLFPGPSKP FWVLVVVGGV LACYSLLVTV AFIIFWVRRD QRLPPDAHKP |
| | | PGGGSFRTPI QEEQADAHST LAKIKKVAKK PTNKAPHPKQ EPQEINFPDD |
| | | LPGSNTAAPV QETLHGCQPV TQEDGKESRI SVQERQ |
| 401 | OSM_MOV19_ | MGVLLTQRTL LSLVLALLFP SMASMQVQLQ QSGAELVKPG ASVKISCKAS |
| | spCD28_ | GYSFTGYFMN WVKQSHGKSL EWIGRIHPYD GDTFYNQNFK DKATLTVDKS |
| | CD2_CD40 | SNTAHMELLS LTSEDFAVYY CTRYDGSRAM DYWGQGTTVT VSSGGGGSGG |
| | | GGSGGGGSDI ELTQSPASLA VSLGQRAIIS CKASQSVSFA GTSLMHWYHQ |
| | | KPGQQPKLLI YRASNLEAGV PTRFSGSGSK TDFTLNIHPV EEEDAATYYC |
| | | QQSREYPYTF GGGTKLEIKA AAGSGGSGIL VKQSPMLVAY DNAVNLSCKY |
| | | SYNLFSREFR ASLHKGLDSA VEVCVVYGNY SQQLQVYSKT GFNCDGKLGN |
| | | ESVTFYLQNL YVNQTDIYFC KIEVMYPPPY LDNEKSNGTI IHVKGKHLCP |
| | | SPLFPGPSKP FWVLVVVGGV LACYSLLVTV AFIIFWVKRK KQRSRRNDEE |
| | | LETRAHRVAT EERGRKPHQI PASTPQNPAT SQHPPPPPGH RSQAPSHRPP |
| | | PPGHRVQHQP QKRPPAPSGT QVHQQKGPPL PRPRVQPKPP HGAAENSLSP |
| | | SSNKKVAKKP TNKAPHPKQE PQEINFPDDL PGSNTAAPVQ ETLHGCQPVT |
| | | QEDGKESRIS VQERQ |
| 402 | OSM_MOV19_ | MGVLLTQRTL LSLVLALLFP SMASMQVQLQ QSGAELVKPG ASVKISCKAS |
| | spCD28_ | GYSFTGYFMN WVKQSHGKSL EWIGRIHPYD GDTFYNQNFK DKATLTVDKS |
| | GITR_CD40 | SNTAHMELLS LTSEDFAVYY CTRYDGSRAM DYWGQGTTVT VSSGGGGSGG |
| | | GGSGGGGSDI ELTQSPASLA VSLGQRAIIS CKASQSVSFA GTSLMHWYHQ |
| | | KPGQQPKLLI YRASNLEAGV PTRFSGSGSK TDFTLNIHPV EEEDAATYYC |
| | | QQSREYPYTF GGGTKLEIKA AAGSGGSGIL VKQSPMLVAY DNAVNLSCKY |
| | | SYNLFSREFR ASLHKGLDSA VEVCVVYGNY SQQLQVYSKT GFNCDGKLGN |
| | | ESVTFYLQNL YVNQTDIYFC KIEVMYPPPY LDNEKSNGTI IHVKGKHLCP |
| | | SPLFPGPSKP FWVLVVVGGV LACYSLLVTV AFIIFWVQLG LHIWQLRSQC |
| | | MWPRETQLLL EVPPSTEDAR SCQFPEEERG ERSAEEKGRL GDLWVKKVAK |
| | | KPTNKAPHPK QEPQEINFPD DLPGSNTAAP VQETLHGCQP VTQEDGKESR |
| | | ISVQERQ |
| 403 | OSM_MOV19_ | MGVLLTQRTL LSLVLALLFP SMASMQVOLQ QSGAELVKPG ASVKISCKAS |
| | spCD28_ | GYSFTGYFMN WVKQSHGKSL EWIGRIHPYD GDTFYNQNFK DKATLTVDKS |
| | CD29_CD40 | SNTAHMELLS LTSEDFAVYY CTRYDGSRAM DYWGQGTTVT VSSGGGGSGG |
| | | GGSGGGGSDI ELTQSPASLA VSLGQRAIIS CKASQSVSFA GTSLMHWYHQ |
| | | KPGQQPKLLI YRASNLEAGV PTRFSGSGSK TDFTLNIHPV EEEDAATYYC |
| | | QQSREYPYTF GGGTKLEIKA AAGSGGSGIL VKQSPMLVAY DNAVNLSCKY |
| | | SYNLFSREFR ASLHKGLDSA VEVCVVYGNY SQQLQVYSKT GFNCDGKLGN |
| | | ESVTFYLQNL YVNQTDIYFC KIEVMYPPPY LDNEKSNGTI IHVKGKHLCP |
| | | SPLFPGPSKP FWVLVVVGGV LACYSLLVTV AFIIFWVKLL MIIHDRREFA |
| | | KFEKEKMNAK WDTGENPIYK SAVTTVVNPK YEGKKKVAKK PTNKAPHPKQ |
| | | EPQEINFPDD LPGSNTAAPV QETLHGCQPV TQEDGKESRI SVQERQ |
| 404 | OSM_MOV19_ | MGVLLTQRTL LSLVLALLFP SMASMQVQLQ QSGAELVKPG ASVKISCKAS |
| | spCD28_ | GYSFTGYFMN WVKQSHGKSL EWIGRIHPYD GDTFYNQNFK DKATLTVDKS |
| | CD150_CD40 | SNTAHMELLS LTSEDFAVYY CTRYDGSRAM DYWGQGTTVT VSSGGGGSGG |
| | | GGSGGGGSDI ELTQSPASLA VSLGQRAIIS CKASQSVSFA GTSLMHWYHQ |
| | | KPGQQPKLLI YRASNLEAGV PTRFSGSGSK TDFTLNIHPV EEEDAATYYC |
| | | QQSREYPYTF GGGTKLEIKA AAGSGGSGIL VKQSPMLVAY DNAVNLSCKY |
| | | SYNLFSREFR ASLHKGLDSA VEVCVVYGNY SQQLQVYSKT GFNCDGKLGN |
| | | ESVTFYLQNL YVNQTDIYFC KIEVMYPPPY LDNEKSNGTI IHVKGKHLCP |
| | | SPLFPGPSKP FWVLVVVGGV LACYSLLVTV AFIIFWVRRR GKTNHYQTTV |
| | | EKKSLTIYAQ VQKPGPLQKK LDSFPAQDPC TTIYVAATEP VPESVQETNS |
| | | ITVYASVTLP ESKKVAKKPT NKAPHPKQEP QEINFPDDLP GSNTAAPVQE |
| | | TLHGCQPVTQ EDGKESRISV QERQ |
| 405 | OSM_MOV19_ | MGVLLTQRTL LSLVLALLFP SMASMQVQLQ QSGAELVKPG ASVKISCKAS |
| | spCD8_CD28_ | GYSFTGYFMN WVKQSHGKSL EWIGRIHPYD GDTFYNQNFK DKATLTVDKS |
| | CD40 | SNTAHMELLS LTSEDFAVYY CTRYDGSRAM DYWGQGTTVT VSSGGGGSGG |
| | | GGSGGGGSDI ELTQSPASLA VSLGQRAIIS CKASQSVSFA GTSLMHWYHQ |
| | | KPGQQPKLLI YRASNLEAGV PTRFSGSGSK TDFTLNIHPV EEEDAATYYC |
| | | QQSREYPYTF GGGTKLEIKA AAGSGGSGFV PVFLPAKPTT TPAPRPPTPA |
| | | PTIASQPLSL RPEACRPAAG GAVHTRGLDF ACDIYIWAPL AGTCGVLLLS |
| | | LVITLYCNHR NRSKRSRLLH SDYMNMTPRR PGPTRKHYQP YAPPRDFAAY |
| | | RSKKVAKKPT NKAPHPKQEP QEINFPDDLP GSNTAAPVQE TLHGCQPVTQ |
| | | EDGKESRISV QERQ |
| 406 | OSM_MOV19 | MGVLLTQRTL LSLVLALLFP SMASMQVQLQ QSGAELVKPG ASVKISCKAS |
| | _spCD8_CD40 | GYSFTGYFMN WVKQSHGKSL EWIGRIHPYD GDTFYNQNFK DKATLTVDKS |
| | | SNTAHMELLS LTSEDFAVYY CTRYDGSRAM DYWGQGTTVT VSSGGGGSGG |
| | | GGSGGGGSDI ELTQSPASLA VSLGQRAIIS CKASQSVSFA GTSLMHWYHQ |
| | | KPGQQPKLLI YRASNLEAGV PTRFSGSGSK TDFTLNIHPV EEEDAATYYC |
| | | QQSREYPYTF GGGTKLEIKA AAGSGGSGFV PVFLPAKPTT TPAPRPPTPA |
| | | PTIASQPLSL RPEACRPAAG GAVHTRGLDF ACDIYIWAPL AGTCGVLLLS |
| | | LVITLYCNHR NKKVAKKPTN KAPHPKQEPQ EINFPDDLPG SNTAAPVQET |
| | | LHGCQPVTQE DGKESRISVQ ERQ |
| 407 | OSM_MOV19_ | MGVLLTQRTL LSLVLALLFP SMASMQVQLQ QSGAELVKPG ASVKISCKAS |
| | spCD8_CD137_ | GYSFTGYFMN WVKQSHGKSL EWIGRIHPYD GDTFYNQNFK DKATLTVDKS |
| | CD40 | SNTAHMELLS LTSEDFAVYY CTRYDGSRAM DYWGQGTTVT VSSGGGGSGG |
| | | GGSGGGGSDI ELTQSPASLA VSLGQRAIIS CKASQSVSFA GTSLMHWYHQ |
| | | KPGQQPKLLI YRASNLEAGV PTRFSGSGSK TDFTLNIHPV EEEDAATYYC |
| | | QQSREYPYTF GGGTKLEIKA AAGSGGSGFV PVFLPAKPTT TPAPRPPTPA |
| | | PTIASQPLSL RPEACRPAAG GAVHTRGLDF ACDIYIWAPL AGTCGVLLLS |
| | | LVITLYCNHR NRFSVVKRGR KKLLYIFKQP FMRPVQTTQE EDGCSCRFPE |
| | | EEEGGCEKKV AKKPTNKAPH PKQEPQEINF PDDLPGSNTA APVQETLHGC |
| | | QPVTQEDGKE SRISVQERQ |
| 408 | OSM_MOV19_ | MGVLLTQRTL LSLVLALLFP SMASMQVQLQ QSGAELVKPG ASVKISCKAS |
| | spCD8_CD134_ | GYSFTGYFMN WVKQSHGKSL EWIGRIHPYD GDTFYNQNFK DKATLTVDKS |
| | CD40 | SNTAHMELLS LTSEDFAVYY CTRYDGSRAM DYWGQGTTVT VSSGGGGSGG |
| | | GGSGGGGSDI ELTQSPASLA VSLGQRAIIS CKASQSVSFA GTSLMHWYHQ |
| | | KPGQQPKLLI YRASNLEAGV PTRFSGSGSK TDFTLNIHPV EEEDAATYYC |
| | | QQSREYPYTF GGGTKLEIKA AAGSGGSGFV PVFLPAKPTT TPAPRPPTPA |
| | | PTIASQPLSL RPEACRPAAG GAVHTRGLDF ACDIYIWAPL AGTCGVLLLS |
| | | LVITLYCNHR NRRDQRLPPD AHKPPGGGSF RTPIQEEQAD AHSTLAKIKK |
| | | VAKKPTNKAP HPKQEPQEIN FPDDLPGSNT AAPVQETLHG CQPVTQEDGK |
| | | SRISVQERQ |
| 409 | OSM_MOV19_ | MGVLLTQRTL LSLVLALLFP SMASMQVQLQ QSGAELVKPG ASVKISCKAS |
| | spCD8_CD2_CD40 | GYSFTGYFMN WVKQSHGKSL EWIGRIHPYD GDTFYNQNFK DKATLTVDKS |
| | | SNTAHMELLS LTSEDFAVYY CTRYDGSRAM DYWGQGTTVT VSSGGGGSGG |
| | | GGSGGGGSDI ELTQSPASLA VSLGQRAIIS CKASQSVSFA GTSLMHWYHQ |
| | | KPGQQPKLLI YRASNLEAGV PTRFSGSGSK TDFTLNIHPV EEEDAATYYC |
| | | QQSREYPYTF GGGTKLEIKA AAGSGGSGFV PVFLPAKPTT TPAPRPPTPA |
| | | PTIASQPLSL RPEACRPAAG GAVHTRGLDF ACDIYIWAPL AGTCGVLLLS |
| | | LVITLYCNHR NKRKKQRSRR NDEELETRAH RVATEERGRK PHQIPASTPQ |
| | | NPATSQHPPP PPGHRSQAPS HRPPPPGHRV QHQPQKRPPA PSGTQVHQQK |
| | | GPPLPRPRVQ PKPPHGAAEN SLSPSSNKKV AKKPTNKAPH PKQEPQEINF |
| | | PDDLPGSNTA APVQETLHGC QPVTQEDGKE SRISVQERQ |
| 410 | OSM_MOV19_ | MGVLLTQRTL LSLVLALLFP SMASMQVOLQ QSGAELVKPG ASVKISCKAS |
| | spCD8_GITR_ | GYSFTGYFMN WVKQSHGKSL EWIGRIHPYD GDTFYNQNFK DKATLTVDKS |
| | CD40 | SNTAHMELLS LTSEDFAVYY CTRYDGSRAM DYWGQGTTVT VSSGGGGSGG |
| | | GGSGGGGSDI ELTQSPASLA VSLGQRAIIS CKASQSVSFA GTSLMHWYHQ |
| | | KPGQQPKLLI YRASNLEAGV PTRFSGSGSK TDFTLNIHPV EEEDAATYYC |
| | | QQSREYPYTF GGGTKLEIKA AAGSGGSGFV PVFLPAKPTT TPAPRPPTPA |
| | | PTIASQPLSL RPEACRPAAG GAVHTRGLDF ACDIYIWAPL AGTCGVLLLS |
| | | LVITLYCNHR NQLGLHIWQL RSQCMWPRET QLLLEVPPST EDARSCQFPE |
| | | EERGERSAEE KGRLGDLWVK KVAKKPTNKA PHPKQEPQEI NFPDDLPGSN |
| | | TAAPVQETLH GCQPVTQEDG KESRISVQER Q |
| 411 | OSM_MOV19_ | MGVLLTQRTL LSLVLALLFP SMASMQVQLQ QSGAELVKPG ASVKISCKAS |
| | spCD8_CD29_ | GYSFTGYFMN WVKQSHGKSL EWIGRIHPYD GDTFYNQNFK DKATLTVDKS |
| | CD40 | SNTAHMELLS LTSEDFAVYY CTRYDGSRAM DYWGQGTTVT VSSGGGGSGG |
| | | GGSGGGGSDI ELTQSPASLA VSLGQRAIIS CKASQSVSFA GTSLMHWYHQ |
| | | KPGQQPKLLI YRASNLEAGV PTRFSGSGSK TDFTLNIHPV EEEDAATYYC |
| | | QQSREYPYTF GGGTKLEIKA AAGSGGSGFV PVFLPAKPTT TPAPRPPTPA |
| | | PTIASQPLSL RPEACRPAAG GAVHTRGLDF ACDIYIWAPL AGTCGVLLLS |
| | | LVITLYCNHR NKLLMIIHDR REFAKFEKEK MNAKWDTGEN PIYKSAVTTV |
| | | VNPKYEGKKK VAKKPTNKAP HPKQEPQEIN FPDDLPGSNT AAPVQETLHG |
| | | CQPVTQEDGK ESRISVQERQ |
| 412 | OSM_MOV19_ | MGVLLTQRTL LSLVLALLFP SMASMQVQLQ QSGAELVKPG ASVKISCKAS |
| | spCD8_CD150_ | GYSFTGYFMN WVKQSHGKSL EWIGRIHPYD GDTFYNQNFK DKATLTVDKS |
| | CD40 | SNTAHMELLS LTSEDFAVYY CTRYDGSRAM DYWGQGTTVT VSSGGGGSGG |
| | | GGSGGGGSDI ELTQSPASLA VSLGQRAIIS CKASQSVSFA GTSLMHWYHQ |
| | | KPGQQPKLLI YRASNLEAGV PTRFSGSGSK TDFTLNIHPV EEEDAATYYC |
| | | QQSREYPYTF GGGTKLEIKA AAGSGGSGFV PVFLPAKPTT TPAPRPPTPA |
| | | PTIASQPLSL RPEACRPAAG GAVHTRGLDF ACDIYIWAPL AGTCGVLLLS |
| | | LVITLYCNHR NRRRGKTNHY QTTVEKKSLT IYAQVQKPGP LQKKLDSFPA |
| | | QDPCTTIYVA ATEPVPESVQ ETNSITVYAS VTLPESKKVA KKPTNKAPHP |
| | | KQEPQEINFP DDLPGSNTAA PVQETLHGCQ PVTQEDGKES RISVQERQ |
| 413 | OSM_MOV19_ | MGVLLTQRTL LSLVLALLFP SMASMQVQLQ QSGAELVKPG ASVKISCKAS |
| | spIG4_CD28_ | GYSFTGYFMN WVKQSHGKSL EWIGRIHPYD GDTFYNQNFK DKATLTVDKS |
| | CD40 | SNTAHMELLS LTSEDFAVYY CTRYDGSRAM DYWGQGTTVT VSSGGGGSGG |
| | | GGSGGGGSDI ELTQSPASLA VSLGQRAIIS CKASQSVSFA GTSLMHWYHQ |
| | | KPGQQPKLLI YRASNLEAGV PTRFSGSGSK TDFTLNIHPV EEEDAATYYC |
| | | QQSREYPYTF GGGTKLEIKA AAGSGGSGES KYGPPCPSCP APEFLGGPSV |
| | | FLFPPKPKDT LMISRTPEVT CVVVDVSQED PEVQFNWYVD GVEVHNAKTK |
| | | PREEQFNSTY RVVSVLTVLH QDWLNGKEYK CKVSNKGLPS SIEKTISKAK |
| | | GQPREPQVYT LPPSQEEMTK NQVSLTCLVK GFYPSDIAVE WESNGQPENN |
| | | YKTTPPVLDS DGSFFLYSRL TVDKSRWQEG NVFSCSVMHE ALHNHYTQKS |
| | | LSLSLGKMFW VLVVVGGVLA CYSLLVTVAF IIFWVRSKRS RLLHSDYMNM |
| | | TPRRPGPTRK HYQPYAPPRD FAAYRSKKVA KKPTNKAPHP KQEPQEINFP |
| | | DDLPGSNTAA PVQETLHGCQ PVTQEDGKES RISVQERQ |
| 414 | OSM_MOV19_ | MGVLLTQRTL LSLVLALLFP SMASMQVOLQ QSGAELVKPG ASVKISCKAS |
| | spIG4_CD40 | GYSFTGYFMN WVKQSHGKSL EWIGRIHPYD GDTFYNQNFK DKATLTVDKS |
| | | SNTAHMELLS LTSEDFAVYY CTRYDGSRAM DYWGQGTTVT VSSGGGGSGG |
| | | GGSGGGGSDI ELTQSPASLA VSLGQRAIIS CKASQSVSFA GTSLMHWYHQ |
| | | KPGQQPKLLI YRASNLEAGV PTRFSGSGSK TDFTLNIHPV EEEDAATYYC |
| | | QQSREYPYTF GGGTKLEIKA AAGSGGSGES KYGPPCPSCP APEFLGGPSV |
| | | FLFPPKPKDT LMISRTPEVT CVVVDVSQED PEVQFNWYVD GVEVHNAKTK |
| | | PREEQFNSTY RVVSVLTVLH QDWLNGKEYK CKVSNKGLPS SIEKTISKAK |
| | | GQPREPQVYT LPPSQEEMTK NQVSLTCLVK GFYPSDIAVE WESNGQPENN |
| | | YKTTPPVLDS DGSFFLYSRL TVDKSRWQEG NVFSCSVMHE ALHNHYTQKS |
| | | LSLSLGKMFW VLVVVGGVLA CYSLLVTVAF IIFWVKKVAK KPTNKAPHPK |
| | | QEPQEINFPD DLPGSNTAAP VQETLHGCQP VTQEDGKESR ISVQERQ |
| 415 | OSM_MOV19_ | MGVLLTQRTL LSLVLALLFP SMASMQVQLQ QSGAELVKPG ASVKISCKAS |
| | spIG4_CD137_ | GYSFTGYFMN WVKQSHGKSL EWIGRIHPYD GDTFYNQNFK DKATLTVDKS |
| | CD40 | SNTAHMELLS LTSEDFAVYY CTRYDGSRAM DYWGQGTTVT VSSGGGGSGG |
| | | GGSGGGGSDI ELTQSPASLA VSLGQRAIIS CKASQSVSFA GTSLMHWYHQ |
| | | KPGQQPKLLI YRASNLEAGV PTRFSGSGSK TDFTLNIHPV EEEDAATYYC |
| | | QQSREYPYTF GGGTKLEIKA AAGSGGSGES KYGPPCPSCP APEFLGGPSV |
| | | FLFPPKPKDT LMISRTPEVT CVVVDVSQED PEVQFNWYVD GVEVHNAKTK |
| | | PREEQFNSTY RVVSVLTVLH QDWLNGKEYK CKVSNKGLPS SIEKTISKAK |
| | | GQPREPQVYT LPPSQEEMTK NQVSLTCLVK GFYPSDIAVE WESNGQPENN |
| | | YKTTPPVLDS DGSFFLYSRL TVDKSRWQEG NVFSCSVMHE ALHNHYTQKS |
| | | LSLSLGKMFW VLVVVGGVLA CYSLLVTVAF IIFWVKKVAK KPTNKAPHPK |
| | | QEPQEINFPD DLPGSNTAAP VQETLHGCQP VTQEDGKESR ISVQERQ |
| 416 | OSM_MOV19_ | MGVLLTQRTL LSLVLALLFP SMASMQVQLQ QSGAELVKPG ASVKISCKAS |
| | spIG4_CD134_ | GYSFTGYFMN WVKQSHGKSL EWIGRIHPYD GDTFYNQNFK DKATLTVDKS |
| | CD40 | SNTAHMELLS LTSEDFAVYY CTRYDGSRAM DYWGQGTTVT VSSGGGGSGG |
| | | GGSGGGGSDI ELTQSPASLA VSLGQRAIIS CKASQSVSFA GTSLMHWYHQ |
| | | KPGQQPKLLI YRASNLEAGV PTRFSGSGSK TDFTLNIHPV EEEDAATYYC |
| | | QQSREYPYTF GGGTKLEIKA AAGSGGSGES KYGPPCPSCP APEFLGGPSV |
| | | FLFPPKPKDT LMISRTPEVT CVVVDVSQED PEVQFNWYVD GVEVHNAKTK |
| | | PREEQFNSTY RVVSVLTVLH QDWLNGKEYK CKVSNKGLPS SIEKTISKAK |
| | | GQPREPQVYT LPPSQEEMTK NQVSLTCLVK GFYPSDIAVE WESNGQPENN |
| | | YKTTPPVLDS DGSFFLYSRL TVDKSRWQEG NVFSCSVMHE ALHNHYTQKS |
| | | LSLSLGKMFW VLVVVGGVLA CYSLLVTVAF IIFWVRRDQR LPPDAHKPPG |
| | | GGSFRTPIQE EQADAHSTLA KIKKVAKKPT NKAPHPKQEP QEINFPDDLP |
| | | GSNTAAPVQE TLHGCQPVTQ EDGKESRISV QERQ |
| 417 | OSM_MOV19_ | MGVLLTQRTL LSLVLALLFP SMASMQVOLQ QSGAELVKPG ASVKISCKAS |
| | spIG4_CD2_CD40 | GYSFTGYFMN WVKQSHGKSL EWIGRIHPYD GDTFYNQNFK DKATLTVDKS |
| | | SNTAHMELLS LTSEDFAVYY CTRYDGSRAM DYWGQGTTVT VSSGGGGSGG |
| | | GGSGGGGSDI ELTQSPASLA VSLGQRAIIS CKASQSVSFA GTSLMHWYHQ |
| | | KPGQQPKLLI YRASNLEAGV PTRFSGSGSK TDFTLNIHPV EEEDAATYYC |
| | | QQSREYPYTF GGGTKLEIKA AAGSGGSGES KYGPPCPSCP APEFLGGPSV |
| | | FLFPPKPKDT LMISRTPEVT CVVVDVSQED PEVQFNWYVD GVEVHNAKTK |
| | | PREEQFNSTY RVVSVLTVLH QDWLNGKEYK CKVSNKGLPS SIEKTISKAK |
| | | GQPREPQVYT LPPSQEEMTK NQVSLTCLVK GFYPSDIAVE WESNGQPENN |
| | | YKTTPPVLDS DGSFFLYSRL TVDKSRWQEG NVFSCSVMHE ALHNHYTQKS |
| | | LSLSLGKMFW VLVVVGGVLA CYSLLVTVAF IIFWVKRKKQ RSRRNDEELE |
| | | TRAHRVATEE RGRKPHQIPA STPQNPATSQ HPPPPPGHRS QAPSHRPPPP |
| | | GHRVQHQPQK RPPAPSGTQV HQQKGPPLPR PRVQPKPPHG AAENSLSPSS |
| | | NKKVAKKPTN KAPHPKQEPQ EINFPDDLPG SNTAAPVQET LHGCQPVTQE |
| | | DGKESRISVQ ERQ |
| 418 | OSM_MOV19_ | MGVLLTQRTL LSLVLALLFP SMASMQVQLQ QSGAELVKPG ASVKISCKAS |
| | spIG4_GITR_CD40 | GYSFTGYFMN WVKQSHGKSL EWIGRIHPYD GDTFYNQNFK DKATLTVDKS |
| | | SNTAHMELLS LTSEDFAVYY CTRYDGSRAM DYWGQGTTVT VSSGGGGSGG |
| | | GGSGGGGSDI ELTQSPASLA VSLGQRAIIS CKASQSVSFA GTSLMHWYHQ |
| | | KPGQQPKLLI YRASNLEAGV PTRFSGSGSK TDFTLNIHPV EEEDAATYYC |
| | | QQSREYPYTF GGGTKLEIKA AAGSGGSGES KYGPPCPSCP APEFLGGPSV |
| | | FLFPPKPKDT LMISRTPEVT CVVVDVSQED PEVQFNWYVD GVEVHNAKTK |
| | | PREEQFNSTY RVVSVLTVLH QDWLNGKEYK CKVSNKGLPS SIEKTISKAK |
| | | GQPREPQVYT LPPSQEEMTK NQVSLTCLVK GFYPSDIAVE WESNGQPENN |
| | | YKTTPPVLDS DGSFFLYSRL TVDKSRWQEG NVFSCSVMHE ALHNHYTQKS |
| | | LSLSLGKMFW VLVVVGGVLA CYSLLVTVAF IIFWVQLGLH IWQLRSQCMW |
| | | PRETQLLLEV PPSTEDARSC QFPEEERGER SAEEKGRLGD LWVKKVAKKP |
| | | TNKAPHPKQE PQEINFPDDL PGSNTAAPVQ ETLHGCQPVT QEDGKESRIS |
| | | VQERQ |
| 419 | OSM_MOV19_ | MGVLLTQRTL LSLVLALLFP SMASMQVQLQ QSGAELVKPG ASVKISCKAS |
| | spIG4_CD29_CD40 | GYSFTGYFMN WVKQSHGKSL EWIGRIHPYD GDTFYNQNFK DKATLTVDKS |
| | | SNTAHMELLS LTSEDFAVYY CTRYDGSRAM DYWGQGTTVT VSSGGGGSGG |
| | | GGSGGGGSDI ELTQSPASLA VSLGQRAIIS CKASQSVSFA GTSLMHWYHQ |
| | | KPGQQPKLLI YRASNLEAGV PTRFSGSGSK TDFTLNIHPV EEEDAATYYC |
| | | QQSREYPYTF GGGTKLEIKA AAGSGGSGES KYGPPCPSCP APEFLGGPSV |
| | | FLFPPKPKDT LMISRTPEVT CVVVDVSQED PEVQFNWYVD GVEVHNAKTK |
| | | PREEQFNSTY RVVSVLTVLH QDWLNGKEYK CKVSNKGLPS SIEKTISKAK |
| | | GQPREPQVYT LPPSQEEMTK NQVSLTCLVK GFYPSDIAVE WESNGQPENN |
| | | YKTTPPVLDS DGSFFLYSRL TVDKSRWQEG NVFSCSVMHE ALHNHYTQKS |
| | | LSLSLGKMFW VLVVVGGVLA CYSLLVTVAF IIFWVKLLMI IHDRREFAKF |
| | | EKEKMNAKWD TGENPIYKSA VTTVVNPKYE GKKKVAKKPT NKAPHPKQEP |
| | | QEINFPDDLP GSNTAAPVQE TLHGCQPVTQ EDGKESRISV QERQ |
| 420 | OSM_MOV19_ | MGVLLTQRTL LSLVLALLFP SMASMQVQLQ QSGAELVKPG ASVKISCKAS |
| | spIG4_CD150_ | GYSFTGYFMN WVKQSHGKSL EWIGRIHPYD GDTFYNQNFK DKATLTVDKS |
| | CD40 | SNTAHMELLS LTSEDFAVYY CTRYDGSRAM DYWGQGTTVT VSSGGGGSGG |
| | | GGSGGGGSDI ELTQSPASLA VSLGQRAIIS CKASQSVSFA GTSLMHWYHQ |
| | | KPGQQPKLLI YRASNLEAGV PTRFSGSGSK TDFTLNIHPV EEEDAATYYC |
| | | QQSREYPYTF GGGTKLEIKA AAGSGGSGES KYGPPCPSCP APEFLGGPSV |
| | | FLFPPKPKDT LMISRTPEVT CVVVDVSQED PEVQFNWYVD GVEVHNAKTK |
| | | PREEQFNSTY RVVSVLTVLH QDWLNGKEYK CKVSNKGLPS SIEKTISKAK |
| | | GQPREPQVYT LPPSQEEMTK NQVSLTCLVK GFYPSDIAVE WESNGQPENN |
| | | YKTTPPVLDS DGSFFLYSRL TVDKSRWQEG NVFSCSVMHE ALHNHYTQKS |
| | | LSLSLGKMFW VLVVVGGVLA CYSLLVTVAF IIFWVRRRGK TNHYQTTVEK |
| | | KSLTIYAQVQ KPGPLQKKLD SFPAQDPCTT IYVAATEPVP ESVQETNSIT |
| | | VYASVTLPES KKVAKKPTNK APHPKQEPQE INFPDDLPGS NTAAPVQETL |
| | | HGCQPVTQED GKESRISVQE RQ |
| 421 | OSM_MOV19_ | MGVLLTQRTL LSLVLALLFP SMASMQVQLQ QSGAELVKPG ASVKISCKAS |
| | spIG4_CD40_ | GYSFTGYFMN WVKQSHGKSL EWIGRIHPYD GDTFYNQNFK DKATLTVDKS |
| | tandem | SNTAHMELLS LTSEDFAVYY CTRYDGSRAM DYWGQGTTVT VSSGGGGSGG |
| | | GGSGGGGSDI ELTQSPASLA VSLGQRAIIS CKASQSVSFA GTSLMHWYHQ |
| | | KPGQQPKLLI YRASNLEAGV PTRFSGSGSK TDFTLNIHPV EEEDAATYYC |
| | | QQSREYPYTF GGGTKLEIKA AAGSGGSGES KYGPPCPSCP APEFLGGPSV |
| | | FLFPPKPKDT LMISRTPEVT CVVVDVSQED PEVQFNWYVD GVEVHNAKTK |
| | | PREEQFNSTY RVVSVLTVLH QDWLNGKEYK CKVSNKGLPS SIEKTISKAK |
| | | GQPREPQVYT LPPSQEEMTK NQVSLTCLVK GFYPSDIAVE WESNGQPENN |
| | | YKTTPPVLDS DGSFFLYSRL TVDKSRWQEG NVFSCSVMHE ALHNHYTQKS |
| | | LSLSLGKMFW VLVVVGGVLA CYSLLVTVAF IIFWVAKKPT NKAPHPKQEP |
| | | QEINFPDDLP GSNTAAPVQE TLHGCQPVTQ EDGKESRISV QERQKKVAKK |
| | | PTNKAPHPKQ EPQEINFPDD LPGSNTAAPV QETLHGCQPV TQEDGKESRI |
| | | SVQERQKKVA |
| 422 | OSM_MOV19_ | MGVLLTQRTL LSLVLALLFP SMASMQVOLQ QSGAELVKPG ASVKISCKAS |
| | spIG4_CD40_ | GYSFTGYFMN WVKQSHGKSL EWIGRIHPYD GDTFYNQNFK DKATLTVDKS |
| | P227A | SNTAHMELLS LTSEDFAVYY CTRYDGSRAM DYWGQGTTVT VSSGGGGSGG |
| | | GGSGGGGSDI ELTQSPASLA VSLGQRAIIS CKASQSVSFA GTSLMHWYHQ |
| | | KPGQQPKLLI YRASNLEAGV PTRFSGSGSK TDFTLNIHPV EEEDAATYYC |
| | | QQSREYPYTF GGGTKLEIKA AAGSGGSGES KYGPPCPSCP APEFLGGPSV |
| | | FLFPPKPKDT LMISRTPEVT CVVVDVSQED PEVQFNWYVD GVEVHNAKTK |
| | | PREEQFNSTY RVVSVLTVLH QDWLNGKEYK CKVSNKGLPS SIEKTISKAK |
| | | GQPREPQVYT LPPSQEEMTK NQVSLTCLVK GFYPSDIAVE WESNGQPENN |
| | | YKTTPPVLDS DGSFFLYSRL TVDKSRWQEG NVFSCSVMHE ALHNHYTQKS |
| | | LSLSLGKMFW VLVVVGGVLA CYSLLVTVAF IIFWVKKVAK KPTNKAAHPK |
| | | QEPQEINFPD DLPGSNTAAP VQETLHGCQP VTQEDGKESR ISVQERQ |
| 423 | PD1_PD1_ | MQIPQAPWPV VWAVLQLGWR PGWFLDSPDR PWNPPTFSPA LLVVTEGDNA |
| | sCD28TM_CD28_ | TFTCSFSNTS ESFVLNWYRM SPSNQTDKLA AFPEDRSQPG QDCRFRVTQL |
| | CD40 | PNGRDFHMSV VRARRNDSGT YLCGAISLAP KAQIKESLRA ELRVTERRAE |
| | (dimeric) | VPTAHCPSPL FPGPSKPFWV LVVVGGVLAC YSLLVTVAFI IFWVRSKRSR |
| | CTP188 | LLHSDYMNMT PRRPGPTRKH YQPYAPPRDF AAYRSKKVAK KPTNKAPHPK |
| | | QEPQEINFPD DLPGSNTAAP VQETLHGCQP VTQEDGKESR ISVQERQ |
| 424 | PD1_PD1_ | MQIPQAPWPV VWAVLQLGWR PGWRPGWFLD SPDRPWNPPT FSPALLVVTE |
| | sCD28TM_CD40 | GDNATFTCSF SNTSESFVLN WYRMSPSNQT DKLAAFPEDR SQPGQDCRFR |
| | | VTQLPNGRDF HMSVVRARRN DSGTYLCGAI SLAPKAQIKE SLRAELRVTE |
| | | RRAEVPTAHC PSPLFPGPSK PFWVLVVVGG VLACYSLLVT VAFIIFWVKK |
| | | VAKKPTNKAP HPKQEPQEIN FPDDLPGSNT AAPVQETLHG CQPVTQEDGK |
| | | ESRISVQERQ |
| 425 | TIGIT_TIGIT_ | MRWCLLLIWA QGLRQAPLAS GMMTGTIETT GNISAEKGGS IILQCHLSST |
| | CD28TM_ | TAQVTQVNWE QQDQLLAICN ADLGWHISPS FKDRVAPGPG LGLTLQSLTV |
| | CD28_CD40 | NDTGEYFCIY HTYPDGTYTG RIFLEVLESS VAEHGARFQI PFWVLVVVGG |
| | | VLACYSLLVT VAFIIFWVRS KRSRLLHSDY MNMTPRRPGP TRKHYQPYAP |
| | | PRDFAAYRSK KVAKKPTNKA PHPKQEPQEI NFPDDLPGSN TAAPVQETLH |
| | | GCQPVTQEDG KESRISVQER Q |
| 426 | TIGIT_TIGIT_ | MRWCLLLIWA QGLRQAPLAS GMMTGTIETT GNISAEKGGS IILQCHLSST |
| | CD28TM_CD40 | TAQVTQVNWE QQDQLLAICN ADLGWHISPS FKDRVAPGPG LGLTLQSLTV |
| | | NDTGEYFCIY HTYPDGTYTG RIFLEVLESS VAEHGARFQI PFWVLVVVGG |
| | | VLACYSLLVT VAFIIFWVKK VAKKPTNKAP HPKQEPQEIN FPDDLPGSNT |
| | | AAPVQETLHG CQPVTQEDGK ESRISVQERQ |
| 427 | OSM_MOV19_PD1_ | MGVLLTQRTL LSLVLALLFP SMASMQVOLQ QSGAELVKPG ASVKISCKAS |
| | sCD28TM_CD28_ | GYSFTGYFMN WVKQSHGKSL EWIGRIHPYD GDTFYNQNFK DKATLTVDKS |
| | CD40 | SNTAHMELLS LTSEDFAVYY CTRYDGSRAM DYWGQGTTVT VSSGGGGSGG |
| | | GGSGGGGSDI ELTQSPASLA VSLGQRAIIS CKASQSVSFA GTSLMHWYHQ |
| | | KPGQQPKLLI YRASNLEAGV PTRFSGSGSK TDFTLNIHPV EEEDAATYYC |
| | | QQSREYPYTF GGGTKLEIKA AAGSGGSGRP GWFLDSPDRP WNPPTFSPAL |
| | | LVVTEGDNAT FTCSFSNTSE SFVLNWYRMS PSNQTDKLAA FPEDRSQPGQ |
| | | DCRFRVTQLP NGRDFHMSVV RARRNDSGTY LCGAISLAPK AQIKESLRAE |
| | | LRVTERRAEV PTAHCPSPLF PGPSKPFWVL VVVGGVLACY SLLVTVAFII |
| | | FWVRSKRSRL LHSDYMNMTP RRPGPTRKHY QPYAPPRDFA AYRSKKVAKK |
| | | PTNKAPHPKQ EPQEINFPDD LPGSNTAAPV QETLHGCQPV TQEDGKESRI |
| | | SVQERQ |
| 428 | OSM_MOV19_PD1_ | MGVLLTQRTL LSLVLALLFP SMASMQVQLQ QSGAELVKPG ASVKISCKAS |
| | sCD28TM_CD40 | GYSFTGYFMN WVKQSHGKSL EWIGRIHPYD GDTFYNQNFK DKATLTVDKS |
| | | SNTAHMELLS LTSEDFAVYY CTRYDGSRAM DYWGQGTTVT VSSGGGGSGG |
| | | GGSGGGGSDI ELTQSPASLA VSLGQRAIIS CKASQSVSFA GTSLMHWYHQ |
| | | KPGQQPKLLI YRASNLEAGV PTRFSGSGSK TDFTLNIHPV EEEDAATYYC |
| | | QQSREYPYTF GGGTKLEIKA AAGSGGSGRP GWFLDSPDRP WNPPTFSPAL |
| | | LVVTEGDNAT FTCSFSNTSE SFVLNWYRMS PSNQTDKLAA FPEDRSQPGQ |
| | | DCRFRVTQLP NGRDFHMSVV RARRNDSGTY LCGAISLAPK AQIKESLRAE |
| | | LRVTERRAEV PTAHCPSPLF PGPSKPFWVL VVVGGVLACY SLLVTVAFII |
| | | FWVKKVAKKP TNKAPHPKQE PQEINFPDDL PGSNTAAPVQ ETLHGCQPVT |
| | | QEDGKESRIS VQERQ |
| 429 | OSM_MOV19_ | MGVLLTQRTL LSLVLALLFP SMASMQVQLQ QSGAELVKPG ASVKISCKAS |
| | TIGIT_sCD28TM_ | GYSFTGYFMN WVKQSHGKSL EWIGRIHPYD GDTFYNQNFK DKATLTVDKS |
| | CD28_CD40 | SNTAHMELLS LTSEDFAVYY CTRYDGSRAM DYWGQGTTVT VSSGGGGSGG |
| | | GGSGGGGSDI ELTQSPASLA VSLGQRAIIS CKASQSVSFA GTSLMHWYHQ |
| | | KPGQQPKLLI YRASNLEAGV PTRFSGSGSK TDFTLNIHPV EEEDAATYYC |
| | | QQSREYPYTF GGGTKLEIKA AAGSGGSGMM TGTIETTGNI SAEKGGSIIL |
| | | QCHLSSTTAQ VTQVNWEQQD QLLAICNADL GWHISPSFKD RVAPGPGLGL |
| | | TLQSLTVNDT GEYFCIYHTY PDGTYTGRIF LEVLESSVAE HGARFQIPFW |
| | | VLVVVGGVLA CYSLLVTVAF IIFWVRSKRS RLLHSDYMNM TPRRPGPTRK |
| | | HYQPYAPPRD FAAYRSKKVA KKPTNKAPHP KQEPQEINFP DDLPGSNTAA |
| | | PVQETLHGCQ PVTQEDGKES RISVQERQ |
| 430 | OSM_MOV19_ | MGVLLTQRTL LSLVLALLFP SMASMQVQLQ QSGAELVKPG ASVKISCKAS |
| | TIGIT_sCD28TM_ | GYSFTGYFMN WVKQSHGKSL EWIGRIHPYD GDTFYNQNFK DKATLTVDKS |
| | CD40 | SNTAHMELLS LTSEDFAVYY CTRYDGSRAM DYWGQGTTVT VSSGGGGSGG |
| | | GGSGGGGSDI ELTQSPASLA VSLGQRAIIS CKASQSVSFA GTSLMHWYHQ |
| | | KPGQQPKLLI YRASNLEAGV PTRFSGSGSK TDFTLNIHPV EEEDAATYYC |
| | | QQSREYPYTF GGGTKLEIKA AAGSGGSGMM TGTIETTGNI SAEKGGSIIL |
| | | QCHLSSTTAQ VTQVNWEQQD QLLAICNADL GWHISPSFKD RVAPGPGLGL |
| | | TLQSLTVNDT GEYFCIYHTY PDGTYTGRIF LEVLESSVAE HGARFQIPFW |
| | | VLVVVGGVLA CYSLLVTVAF IIFWVKKVAK KPTNKAPHPK QEPQEINFPD |
| | | DLPGSNTAAP VQETLHGCQP VTQEDGKESR ISVQERQ |
| 431 | Linker | GGGGSGGGGS GGGGS |
| 432 | Truncated | NKILVKQSPM LVAYDNAVNL SCKYSYNLFS REFRASLHKG LDSAVEVCVV |
| | Cytoplasmic | YGNYSQQLQV YSKTGFNCDG KLGNESVTFY LQNLYVNQTD IYFCKIEVMY |
| | domain CD28 | PPPYLDNEKS NGTIIHVKGK HLCPSPLFPG PSKPFWVLVV VGGVLACYSL |
| | Variant | LVTVAFIIFW VRSKR |
| 433 | CD28.CD137 | NKILVKQSPM LVAYDNAVNL SCKYSYNLFS REFRASLHKG LDSAVEVCVV |
| | fusion | YGNYSQQLQV YSKTGFNCDG KLGNESVTFY LQNLYVNQTD IYFCKIEVMY |
| | | PPPYLDNEKS NGTIIHVKGK HLCPSPLFPG PSKPFWVLVV VGGVLACYSL |
| | | LVTVAFIIFW VRSKRSRLLH SDYMNMTPRR PGPTRKHYQP YAPPRDFAAY |
| | | RSRFSVVKRG RKKLLYIFKQ PFMRPVQTTQ EEDGCSCRFP EEEEGGCE |
| 434 | CD28.CD134 | NKILVKQSPM LVAYDNAVNL SCKYSYNLFS REFRASLHKG LDSAVEVCVV |
| | fusion | YGNYSQQLQV YSKTGFNCDG KLGNESVTFY LQNLYVNQTD IYFCKIEVMY |
| | | PPPYLDNEKS NGTIIHVKGK HLCPSPLFPG PSKPFWVLVV VGGVLACYSL |
| | | LVTVAFIIFW VRSKRSRLLH SDYMNMTPRR PGPTRKHYQP YAPPRDFAAY |
| | | RSRRDQRLPP DAHKPPGGGS FRTPIQEEQA DAHSTLAKI |
| 435 | CD28.CD2 fusion | NKILVKQSPM LVAYDNAVNL SCKYSYNLFS REFRASLHKG LDSAVEVCVV |
| | | YGNYSQQLQV YSKTGFNCDG KLGNESVTFY LQNLYVNQTD IYFCKIEVMY |
| | | PPPYLDNEKS NGTIIHVKGK HLCPSPLFPG PSKPFWVLVV VGGVLACYSL |
| | | LVTVAFIIFW VRSKRSRLLH SDYMNMTPRR PGPTRKHYQP YAPPRDFAAY |
| | | RSKRKKQRSR RNDEELETRA HRVATEERGR KPHQIPASTP QNPATSQHPP |
| | | PPPGHRSQAP SHRPPPPGHR VQHQPQKRPP APSGTQVHQQ KGPPLPRPRV |
| | | QPKPPHGAAE NSLSPSSN |
| 436 | CD28.CD29 | NKILVKQSPM LVAYDNAVNL SCKYSYNLFS REFRASLHKG LDSAVEVCVV |
| | fusion | YGNYSQQLQV YSKTGFNCDG KLGNESVTFY LQNLYVNQTD IYFCKIEVMY |
| | | PPPYLDNEKS NGTIIHVKGK HLCPSPLFPG PSKPFWVLVV VGGVLACYSL |
| | | LVTVAFIIFW VRSKRSRLLH SDYMNMTPRR PGPTRKHYQP YAPPRDFAAY |
| | | RSKLLMIIHD RREFAKFEKE KMNAKWDTGE NPIYKSAVTT VVNPKYEGK |
| 437 | CD28.GITR | NKILVKQSPM LVAYDNAVNL SCKYSYNLFS REFRASLHKG LDSAVEVCVV |
| | fusion | YGNYSQQLQV YSKTGFNCDG KLGNESVTFY LQNLYVNQTD IYFCKIEVMY |
| | | PPPYLDNEKS NGTIIHVKGK HLCPSPLFPG PSKPFWVLVV VGGVLACYSL |
| | | LVTVAFIIFW VRSKRSRLLH SDYMNMTPRR PGPTRKHYQP YAPPRDFAAY |
| | | RSQLGLHIWQ LRSQCMWPRE TQLLLEVPPS TEDARSCQFP EEERGERSAE |
| | | EKGRLGDLWV |
| 438 | CD28.IL2Rγ | NKILVKQSPM LVAYDNAVNL SCKYSYNLFS REFRASLHKG LDSAVEVCVV |
| | fusion | YGNYSQQLQV YSKTGFNCDG KLGNESVTFY LQNLYVNQTD IYFCKIEVMY |
| | | PPPYLDNEKS NGTIIHVKGK HLCPSPLFPG PSKPFWVLVV VGGVLACYSL |
| | | LVTVAFIIFW VRSKRSRLLH SDYMNMTPRR PGPTRKHYQP YAPPRDFAAY |
| | | RSERTMPRIP TLKNLEDLVT EYHGNFSAWS GVSKGLAESL QPDYSERLCL |
| | | VSEIPPKGGA LGEGPGASPC NQHSPYWAPP CYTLKPET |
| 439 | CD28.CD40 | NKILVKQSPM LVAYDNAVNL SCKYSYNLFS REFRASLHKG LDSAVEVCVV |
| | fusion | YGNYSQQLQV YSKTGFNCDG KLGNESVTFY LQNLYVNQTD IYFCKIEVMY |
| | | PPPYLDNEKS NGTIIHVKGK HLCPSPLFPG PSKPFWVLVV VGGVLACYSL |
| | | LVTVAFIIFW VRSKRSRLLH SDYMNMTPRR PGPTRKHYQP YAPPRDFAAY |
| | | RSKKVAKKPT NKAPHPKQEP QEINFPDDLP GSNTAAPVQE TLHGCQPVTQ |
| | | EDGKESRISV QERQ |
| 440 | CD28.CD150 | NKILVKQSPM LVAYDNAVNL SCKYSYNLFS REFRASLHKG LDSAVEVCVV |
| | fusion | YGNYSQQLQV YSKTGFNCDG KLGNESVTFY LQNLYVNQTD IYFCKIEVMY |
| | | PPPYLDNEKS NGTIIHVKGK HLCPSPLFPG PSKPFWVLVV VGGVLACYSL |
| | | LVTVAFIIFW VRSKRSRLLH SDYMNMTPRR PGPTRKHYQP YAPPRDFAAY |
| | | RSRRRGKTNH YQTTVEKKSL TIYAQVQKPG PLQKKLDSFP AQDPCTTIYV |
| | | AATEPVPESV QETNSITVYA SVTLPES |
| 441 | CD28.CD2.CD40 | NKILVKQSPM LVAYDNAVNL SCKYSYNLFS REFRASLHKG LDSAVEVCVV |
| | fusion | YGNYSQQLQV YSKTGFNCDG KLGNESVTFY LQNLYVNQTD IYFCKIEVMY |
| | | PPPYLDNEKS NGTIIHVKGK HLCPSPLFPG PSKPFWVLVV VGGVLACYSL |
| | | LVTVAFIIFW VRSKRSRLLH SDYMNMTPRR PGPTRKHYQP YAPPRDFAAY |
| | | RSKRKKQRSR RNDEELETRA HRVATEERGR KPHQIPASTP QNPATSQHPP |
| | | PPPGHRSQAP SHRPPPPGHR VQHQPQKRPP APSGTQVHQQ KGPPLPRPRV |
| | | QPKPPHGAAE NSLSPSSNKK VAKKPTNKAP HPKQEPQEIN FPDDLPGSNT |
| | | AAPVQETLHG CQPVTQEDGK ESRISVQERQ |
| 442 | CD28(IEV) | IEVMYPPPYL DNEKSNGTII HVKGKHLCPS PLFPGPSKPF WVLVVVGGVL |
| | Variant | ACYSLLVTVA FIIFWVRSKR SRLLHSDYMN MTPRRPGPTR KHYQPYAPPR |
| | | DFAAYRS |
| 443 | SH3 motif2 | PTNKAPHP |
| 444 | SH3 motif3 | PTNKAPH |
| 445 | TRAF2_motif4 | PKQET |
| 446 | TRAF2_motif5 | PVQET |
| 447 | TRAF2_motif6 | SVQET |
| 448 | TRAF6-Motif2 | QEPQEINF |
| 449 | HIS tag | DYKDDDDK |

[symbol]

indicates data missing or illegible when filed

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

Embodiments of the present disclosure provided herein are described by way of the following exemplary numbered arrangements:

1. An chimeric costimulatory antigen receptor (CoStAR) which comprises:

an extracellular binding domain that binds to carcinoembryonic antigen (CEA), or an extracellular binding domain that binds to mesothelin (MSLN), operatively linked to a transmembrane domain, and

a first signaling domain and an intracellular domain of ICOS or a signaling fragment thereof, or

a first signaling domain and an intracellular domain of NTRK1 or a signaling fragment thereof, or

a first signaling domain and an intracellular domain of DAP10 or a signaling fragment thereof, or

a first signaling domain and a CD40 signaling domain or a signaling fragment thereof, or

a first signaling domain and one or more of a TRAF2/TRAF3 sequence, a TRAF6 sequence, a TRAF2 sequence, or an IProx sequence.

2. The CoStAR of arrangement 1, wherein the first signaling domain comprises a signaling domain or signaling fragment of CD2, CD9, CD26, CD27, CD28, CD29, CD38, CD40, CD43, CD46, CD49d, CD55, CD73, CD81, CD82, CD99, CD100, CD134 (OX40), CD137 (41BB), CD150 (SLAM), CD270 (HVEM), CD278 (ICOS), CD357 (GITR), or EphB6.

3. The CoStAR of arrangement 1, wherein the CoStAR comprises a second signaling domain.

4. The CoStAR of arrangement 3, wherein the second signaling domain comprises a signaling domain or signaling fragment of CD2, CD9, CD26, CD27, CD28, CD29, CD38, CD40, CD43, CD46, CD49d, CD55, CD73, CD81, CD82, CD99, CD100, CD134 (OX40), CD137 (41BB), CD150 (SLAM), CD270 (HVEM), CD278 (ICOS), CD357 (GITR), or EphB6.

5. The CoStAR of arrangement 1, wherein the CD40 signaling fragment comprises an SH3 motif (KPTNKAPH, SEQ ID NO:35), TRAF2 motif (PKQE, SEQ ID NO:36, PVQE, SEQ ID NO:37, SVQE, SEQ ID NO:38), TRAF6 motif (QEPQEINFP, SEQ ID NO:39), PKA motif (KKPTNKA, SEQ ID NO:40, SRISVQE, SEQ ID NO:41), or a combination thereof, or is a full length CD40 intracellular domain.

6. The CoStAR of arrangement 2, wherein the first signaling domain comprises a full length costimulatory domain.

7. The CoStAR of arrangement 1, wherein the extracellular binding domain is operatively linked to the transmembrane domain by a linker and/or a spacer.

8. The CoStAR of arrangement 7, wherein the linker comprises from about 5 to about 20 amino acids.

9. The CoStAR of arrangement 7, wherein the linker comprises from about 10 to about 250 amino acids.

10. The CoStAR of arrangement 1, wherein the CoStAR comprises a second extracellular binding domain.

11. The CoStAR of arrangement 10, wherein the second extracellular binding domain comprises a ligand binding domain from CD8, CD28, or ICOS.

12. The CoStAR of arrangement 1, wherein the transmembrane domain comprises a transmembrane domain from CD28, CD8, ICOS, DAP10, or NTRK.

13. The CoStAR of arrangement 1, wherein the transmembrane domain comprises the transmembrane domain sequence of SEQ ID NO:20, SEQ ID NO:21, or SEQ ID NO:22.

14. The CoStAR of arrangement 1, wherein the extracellular binding domain comprises an scFv, a peptide, an antibody heavy-chain variable domain, an antibody light-chain variable domain, or a CEA ligand.

15. The CoStAR of any one of arrangements 1 to 15, which further comprises a CD3ζ signaling domain at the C-terminus.

16. The CoStAR of any one of arrangements 1 to 14, which further comprises an N-terminal signal peptide.

17. The CoStAR of arrangement 17, wherein the N-terminal signal peptide comprises the signal peptide of oncostatin M (OSM), CD8a, CD2, interleukin-2 (IL-2), granulocyte-macrophage colony stimulating factor (GM-CSF), or human IgGκ.

18. A nucleic acid which encodes the CoStAR of any one of arrangements 1 to 17.

19. A vector which comprises the nucleic acid of arrangement 18.

20. A cell which expresses the CoStAR of any one of arrangements 1 to 16.

21. The cell of arrangement 20, wherein the cell comprises an alpha-beta T cell, gamma-delta T cell, T regulatory cell, TIL, NKT cell or NK cell.

22. The cell of arrangement 20, wherein the cell coexpresses a CAR or a TCR.

23. A method of making the cell of arrangement 20, which comprises the step of transducing or transfecting a cell with a vector of arrangement 19.

24. A method for preparing a population of cells that express a CoStAR of any one of arrangements 1 to 16, which comprises
i) detecting expression of the CoStAR on the surface of cells transfected or transduced with a vector of arrangement 19; and
ii) selecting cells which are identified as expressing the CoStAR.

25. A cell population which is enriched for cell expression a CoStAR of any one of arrangements 1 to 16.

26. A method for treating a disease in a subject, which comprises the step of administering a cell according to any of arrangements 20 to 22, or a cell population according to arrangement 25 to the subject.

27. A protein comprising an amino acid sequence comprising a sequence of Consensus A, or X₁X₂X₃X₄X₅X₆X₇X₈X₉X₁₀X₁₁, wherein:
a. X₁ is any amino acid,
b. X₂ is any amino acid,
c. X₃ is P, X₄ is any amino acid,
d. X₅ is Q,
e. X₆ is C or A,
f. X₇ is any amino acid,
g. X₈ is any amino acid,
h. X₉ is any amino acid,
i. X₁₀ is any amino acid, and
j. X₁₁ is any amino acid.

28. The protein of arrangement 27, wherein the protein is part of a TRAF domain.

29. The protein of any of the preceding arrangements, wherein the protein is part of a TRAF2 domain.

30. The protein of any of the preceding arrangements, wherein:
a. the sequence comprises:
i. X₁ is S or P,
ii. X₂ is V, H, or Y,
iii. X₄ is I, Q, V,
iv. X₇ is T or S,
V. X₈ is D,
vi. X₉ is K, D, or G,
vii. X₁₀ is T, S, G, or A, and
viii. X₁₁ is D, S, N, or D; or
b. the sequence is at least 60% identical to a).

31. A protein comprising an amino acid sequence comprising a sequence of Consensus B, or X₁X₂X₃X₄X₅X₆X₇X₈X₉X₁₀X₁₁, wherein:
a. X₁ is any amino acid,
b. X₂ is any amino acid,
c. X₃ is P, S, A, or T,
d. X₄ is any amino acid,
e. X₅ is Q or E,
f. X₆ is E,
g. X₇ is any amino acid,
h. X₈ is any amino acid,
i. X₉ is any amino acid,
j. X₁₀ is any amino acid, and
k. X₁₁ is any amino acid.

32. The protein of arrangement 31, wherein the protein is part of a TRAF domain.

33. The protein of any of the preceding arrangements, wherein the protein is part of a TRAF2 domain.

34. The protein of any of the preceding arrangements, wherein:
a. the sequence comprises:
i. X₁ is P, A, M, T, S, R, V, H,
ii. X₂ is F, A, L, I, T, V, G, C, A, F, P,
iii. X₄ is K, V, I, H, A, Q, T, E, S,
iv. X₇ is C, T, E, D, S, A,
v. X₈ is A, L, G, Y, I, Q, D, E,
vi. X₉ is F, H, K, R, P, A, G, Y, E, N,
vii. X₁₀ is R, G, E, K, A, S, D, C, C, P, V, and
viii. X₁₁ is S, C, D, P, W, T, A, G, E; or
b. the sequence is at least 60% identical to a).

35. The protein of any one of the preceding arrangements, wherein the sequence is part of a Traf 2/3 sequence.

36. A protein comprising an amino acid sequence comprising a sequence of Consensus C, or X₁X₂X₃X₄X₅X₆, wherein:
a. X₁ is P,
b. X₂ is any amino acid,
c. X₃ is E,
d. X₄ is any amino acid,
e. X₅ is any amino acid, and
f. X₆ is Ac/Ar.

37. The protein of arrangement 36, wherein the protein is part of a TRAF domain.

38. The protein of any of the preceding arrangements, wherein the protein is part of a TRAF6 domain.

39. The protein of any of the preceding arrangements, wherein
a. the sequence comprises:
i. X₂ is Q, P, E, V, T, or S,
ii. X₄ is I, L M, N, S, T, N, D,
iii. X₅ is N, D, R, S, G, or Y; or
b. the sequence is at least 60% identical to a).

40. An amino acid sequence comprising:
a) one or more of SEQ ID NOs: 475, 476, 477, 478, 479, 494, 495, 496, 498, 505, 510, 511, 512, 513, 514, 515, 516, 517, 518, or 519;
b) a sequence that is at least 70% identical to a sequence in a); or
c) a sequence that is at least 80% similar to a).

41. A TRAF domain in a CD40 protein, wherein the TRAF domain comprises one or more of:
a) SEQ ID NOs: 475, 476, 477, 478, 479, 494, 495, 496, 498, 505, 510, 511, 512, 513, 514, 515, 516, 517, 518, or 519;
b) a sequence that is at least 70% identical to a sequence in a); or
c) a sequence that is at least 80% similar to a).

42. A TRAF6 domain sequence comprising: PQEINF, PEEMSW, PPENYE, or PQENSY.

43. A TRAF2/3 domain sequence comprising: PVQET, TQEET, SKEET, AVEET, or PVQET.

44. A TRAF2 domain sequence comprising: SVQE, AVEE or PEEE.

45. A TRAF 6 or TRAF2/3 domain that comprises:
a) the amino acid sequence of one of SEQ ID NOs: 475, 476, 477, 478, 479, 494, 495, 496, 498, 505, 510, 511, 512, 513, 514, 515, 516, 517, 518, or 519;
b) the amino acid sequence that is at least 70% identical to a) or
c) the amino acid sequence that is at least 80% similar to a).

46. The protein, amino acid sequence, or TRAF domain of any one of the preceding arrangements, wherein the sequence is within a CD40 protein sequence.

47. The protein, amino acid sequence, or TRAF domain of any one of the preceding arrangements, wherein the sequence is within a CD40 protein and is located at a corresponding TRAF 2, TRAF2/3, and/or TRAF6 domain within the CD40 protein.

48. A TRAF2/TRAF3 domain that comprises an amino acid sequence of Consensus A, or X₁X₂X₃X₄X₅X₆X₇X₈X₉X₁₀X₁₁, wherein
a. X₁ is any amino acid,
b. X₂ is any amino acid,
c. X₃ is P, X₄ is any amino acid,
d. X₅ is Q,
e. X₆ is C or A,
f. X₇ is any amino acid,
g. X₈ is any amino acid,
h. X₉ is any amino acid,
i. X₁₀ is any amino acid, and
j. X₁₁ is any amino acid.

49. A TRAF2/TRAF3 domain that comprises an amino acid sequence of Consensus B, or X₁X₂X₃X₄X₅X₆X₇X₈X₉X₁₀X₁₁, wherein
a. X₁ is any amino acid,
b. X₂ is any amino acid,
c. X₃ is P, S, A, or T,
d. X₄ is any amino acid,
e. X₅ is Q or E,
f. X₆ is E,
g. X₇ is any amino acid,
h. X₈ is any amino acid,
i. X₉ is any amino acid,
j. X₁₀ is any amino acid, and
k. X₁₁ is any amino acid.

50. A TRAF6 domain that comprises an amino acid sequence of Consensus C, or X₁X₂X₃X₄X₅X₆, wherein
a. X₁ is P,
b. X₂ is any amino acid,
c. X₃ is E,
d. X₄ is any amino acid,
e. X₅ is any amino acid, and
f. X₆ is Ac/Ar.

51. A CD40 domain that comprises an amino acid sequence of one or more of the following:
a. A TRAF2/TRAF3 domain that comprises an amino acid sequence of Consensus A, or X₁X₂X₃X₄X₅X₆X₇X₈X₉X₁₀X₁₁, wherein
i. X₁ is any amino acid,
ii. X₂ is any amino acid,
iii. X₃ is P, X₄ is any amino acid,
iv. X₅ is Q,
v. X₆ is C or A,
vi. X₇ is any amino acid,
vii. X₈ is any amino acid,
viii. X₉ is any amino acid,
ix. X₁₀ is any amino acid, and
x. X₁₁ is any amino acid.
b. A TRAF2/TRAF3 domain that comprises an amino acid sequence of Consensus B, or X₁X₂X₃X₄X₅X₆X₇X₈X₉X₁₀X₁₁, wherein
i. X₁ is any amino acid,
ii. X₂ is any amino acid,
iii. X₃ is P, S, A, or T,
iv. X₄ is any amino acid,
v. X₅ is Q or E,
vi. X₆ is E,
vii. X₇ is any amino acid,
viii. X₈ is any amino acid,
ix. X₉ is any amino acid,
x. X₁₀ is any amino acid, and
xi. X₁₁ is any amino acid.
c. A TRAF6 domain that comprises an amino acid sequence of Consensus C, or X₁X₂X₃X₄X₅X₆, wherein
i. X₁ is P,
ii. X₂ is any amino acid,
iii. X₃ is E,
iv. X₄ is any amino acid,
v. X₅ is any amino acid, and
vi. X₆ is Ac/Ar.

52. An chimeric costimulatory antigen receptor (CoStAR) which comprises:
an extracellular binding domain that binds to carcinoembryonic antigen (CEA), or an extracellular binding domain that binds to mesothelin (MSLN);
a transmembrane domain that is linked to the extracellular binding domain,
a first signaling domain; and
a second signaling domain, wherein the second signaling domain comprises a variant CD40 signaling domain or a signaling fragment thereof, wherein the variant CD40 comprises a sequence of a variant TRAF2/TRAF3 sequence, or a variant TRAF6 sequence, or a variant TRAF2 sequence, wherein the variant CD40 does not comprise SEQ ID NO: 42.

53. The CoStAR of arrangement 52, wherein the first signalling domain comprises a signalling domain or signaling fragment of CD28 or CD278 (ICOS).

54. The CoStAR of arrangement 52, wherein the first signalling domain comprises a full-length costimulatory domain.

55. The CoStAR of arrangement 52, wherein the extracellular binding domain is operatively linked to the transmembrane domain by a linker and/or a spacer.

56. The CoStAR of arrangement 55, wherein the linker comprises from about 5 to about 20 amino acids.

57. The CoStAR of arrangement 55, wherein the linker comprises from about 10 to about 250 amino acids.

58. The CoStAR of arrangement 55, wherein the transmembrane domain comprises a transmembrane domain from CD28, CD8, ICOS, DAP10, or NTRK.

59. The CoStAR of arrangement 55, wherein the transmembrane domain comprises the transmembrane domain sequence of SEQ ID NO:20, SEQ ID NO:21, or SEQ ID NO:22.

60. The CoStAR of arrangement 55, wherein the extracellular binding domain comprises an scFv, a peptide, an antibody heavy-chain variable domain, an antibody light-chain variable domain, or a CEA ligand.

61. A nucleic acid which encodes the CoStAR of any one of arrangements 27 to 60.

62. A vector which comprises the nucleic acid of arrangement 61.

63. A cell which expresses the CoStAR of any one of arrangements 27 to 62.

64. The cell of arrangement 63, wherein the cell comprises an alpha-beta T cell, gamma-delta T cell, T regulatory cell, TIL, NKT cell or NK cell.

65. The cell of arrangement 64, wherein the cell coexpresses a CAR or a TCR.

66. A method of making the cell of any one of arrangements 63-65, which comprises the step of transducing or transfecting a cell with a vector of arrangement 62.

67. A method for preparing a population of cells that express a CoStAR of any one of arrangements 52-60, which comprises:
a. detecting expression of the CoStAR on the surface of cells transfected or transduced with a vector of arrangement 62; and
b. selecting cells which are identified as expressing the CoStAR.

68. A cell population which is enriched for cell expression a CoStAR of any one of arrangements 52-60.

69. A method for treating a disease in a subject, which comprises the step of administering a cell according to any of arrangements 63-65, or a cell population according to arrangement 68 to the subject.

70. A protein comprising the amino acid sequence of one of SEQ ID NOs: 510-519.

71. The protein of arrangement 70, wherein the protein comprises the sequence of SEQ ID NO: 510.

72. The protein of arrangement 70, wherein the protein comprises the sequence of SEQ ID NO: 511.

73. The protein of arrangement 70, wherein the protein comprises the sequence of SEQ ID NO: 512.

74. The protein of arrangement 70, wherein the protein comprises the sequence of SEQ ID NO: 513.

75. The protein of arrangement 70, wherein the protein comprises the sequence of SEQ ID NO: 514.

76. The protein of arrangement 70, wherein the protein comprises the sequence of SEQ ID NO: 515.

77. The protein of arrangement 70, wherein the protein comprises the sequence of SEQ ID NO: 516.

78. The protein of arrangement 70, wherein the protein comprises the sequence of SEQ ID NO: 517.

79. The protein of arrangement 70, wherein the protein comprises the sequence of SEQ ID NO: 518.

80. The protein of arrangement 70, wherein the protein comprises the sequence of SEQ ID NO: 519.

### EXAMPLES

### Example 1—Production of T-Cells Expressing CoStAR

Materials and Methods

Construct design—The MFE23 CoStAR consists of a CEA-specific MFE23, humanized (hu) MFE23, CEA6, BW431/26 or huT84.66 derived single chain antibody fragment nucleotide sequence with an oncostatin M1, CD8α, CD2, IL-2, GM-CSF or hIgGκ VIII leader sequence. Each CoStAR has an extracellular spacer domain derived from CD8 or CD28 or truncated CD28 and a signalling domain derived from CD28 and CD40. The constructs were cloned into pSF.Lenti (Oxford Genetics) containing an MND promoter, and separated from a truncated CD34 marker gene via a P2A cleavage sequence.

Lentiviral Production—Lentiviral production was performed using a three-plasmid packaging system (Cell Biolabs, San Diego, USA) by mixing 10 μg of each plasmid, plus 10 μg of the pSF.Lenti lentiviral plasmid containing the transgene, together in serum free RPMI containing 50 mM CaCl₂). The mixture was added dropwise to a 50% confluent monolayer of 293T cells in 75 cm² flasks. The viral supernatants were collected at 48 and 72 h post transfection, pooled and concentrated using LentiPac lentiviral supernatant concentration (GeneCopoeia, Rockville, Md., USA) solution according to the manufacturer's instructions. Lentiviral supernatants were concentrated 10-fold and used to directly infect primary human T-cells in the presence of 4 g/ml polybrene (Sigma-Aldrich, Dorset, UK). Peripheral blood mononuclear cells were isolated from normal healthy donors before activation for 24 hours with T-cell activation and expansion beads (Invitrogen) according to the manufacturer's instructions before addition of lentiviral supernatants.

Cell transduction was assessed 96 hours post infection using CEA.hFc protein and anti-hFc-PE secondary, plus anti-CD34-APC or by anti-CD34-PE antibodies alone. Cells were then expanded further using ×10 donor mismatched irradiated PBMC feeders at a 1:20-1:200 ratio in RPMI+10% FCS with the addition of 1 μg/ml PHA and 200 IU/ml IL-2. After 14 days the cells were stained as previous and stored ready for assay.

Functionality assays were performed by mixing CoStAR positive or negative cells with wild-type or OKT3 engineered CEA-Positive LoVo or LS174T cells. Briefly, T-cells were mixed with LoVo cells at varying ratios in 96-well plates and IFNγ or IL-2 measured by ELISA. The remaining cells were incubated with 1:10 dilution of WST-1 reagent (Sigma, UK) for 30 min before absorbance reading at 450 nm. % Cytotoxicity was determined using the following equation=100−((Experimental reading−T-cells alone)/(tumour alone))×100.

Proliferation assays were performed by first loading T-cells with 10 μM eFluor450 proliferation dye (eBioscience, UK) for 10 min at 37° C. at a concentration of 1×10⁷ cells/ml before incubating the cells in 5 volumes of cold T-cell media for 5 min on ice. Cells were then washed excessively to remove unbound dye and added to cocultures containing tumour cells. Cells were removed at 2, 6 and 10 days, 1:200 dilution of DRAQ7 added and the cells analysed using a MACSQuant cytometer and MACSQuantify software.

Cell counts for proliferation assays were performed by taking cells from the wells and staining with anti-CD2 PerCP eFluor710 antibody (eBioscience, UK) for 20 min in the dark, followed by DRAQ7 staining and counts made using a MACSQuant analyser.

Results

Primary human T-cells were isolated from Buffy coats obtained from the NHSBT. T-cells were isolated by Ficoll-mediated isolation and T-cell negative isolation kits (StemCell Technologies). The isolated T-cells were activated with human T-cell activation and expansion beads (Invitrogen, UK). Cells were incubated with concentrated lentiviral particles and expanded over a number of days. The lentivirus contained the DNA sequence of the MFE.CoStAR.2A.tCD34 construct (MFE23.scFv fused to full length human CD28 co-expressed with truncated human CD34 via a 2A cleavage sequence). Successfully transduced cells were further expanded using irradiated feeders as outlined in materials and methods. Donor 1 transduction was measured at 22.69% (17.15 CD34+/CoStAR+ plus 5.53% CD34−/CoStAR+), donor 2 was measured at 20.73%, and donor 3 at 13.34%. Cells were enriched for CoStAR expression using anti-CD34 antibodies to obtain T-cell populations greater than 90% CoStAR positive.

To generate a physiologically relevant in vitro model to test the impact of CoStAR on T-cell activity, the non-transduced and transduced cells were tested against the CEA+ tumour cell lines LoVo and LS174T. To enable activation of the T-cells in response to the unmatched tumour lines we engineered the tumour cells to express an anti-CD3 single chain antibody fragment anchored to the cell membrane by way of a synthetic transmembrane domain and split from the GFP marker gene using an IRES element to visualise transduced cells using flow cytometry.

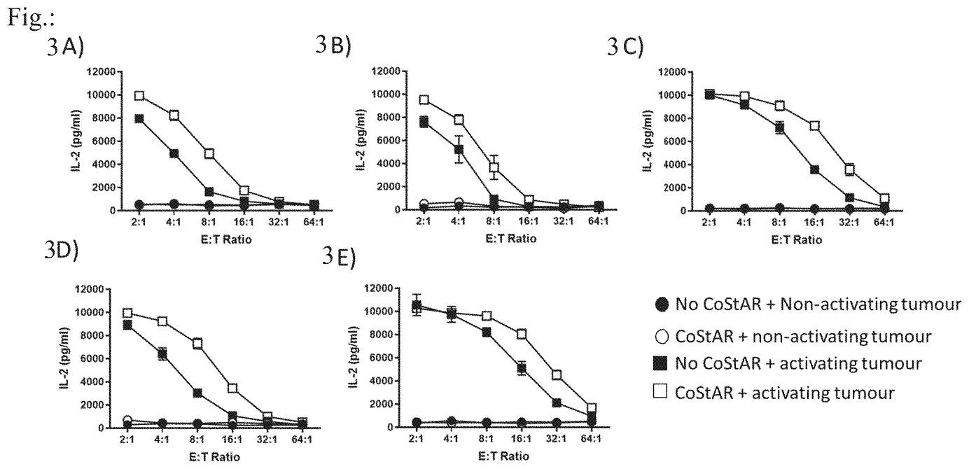

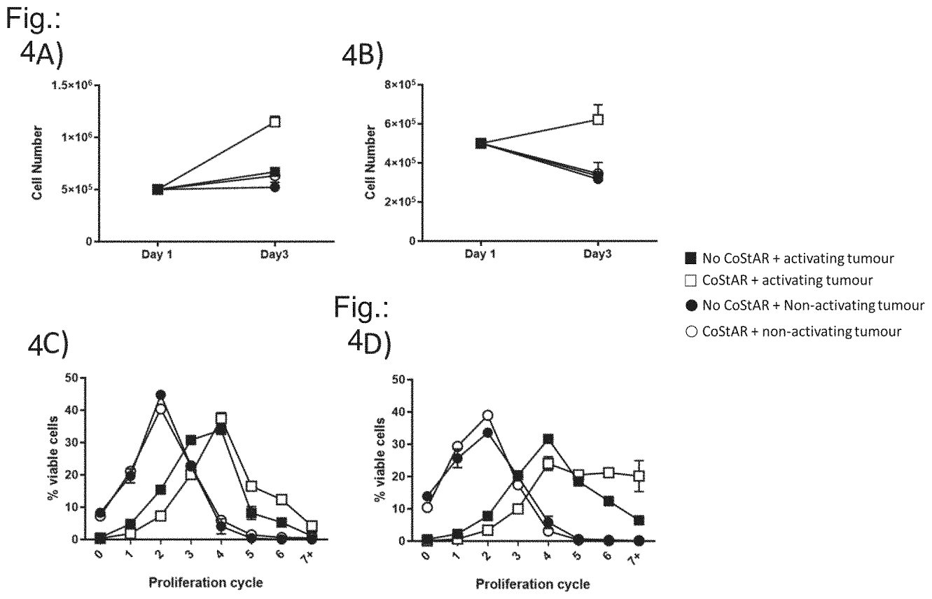

Single cell clones of LoVo and LS174T were generated from bulk transfectants. Non-transduced and CoStAR transduced T-cells were mixed at varying effector:target ratios with wild-type non-transduced or OKT3-engineered LS174T or LoVo cells. After 24 hours coculture media was taken for IL-2 ELISA measurement. Activation dependent IL-2 secretion was observed from both CoStAR+ and CoStAR− T-cell populations from three donors in response to OKT3 engineered LS174T cells with only background IL-2 secretion seen from transduced and non-transduced T-cells in response to un-engineered tumour cells (FIG. 3 A-C). CoStAR enhanced IL-2 secretion towards OKT3 engineered tumour cells was found in all three donors tested. The effect was most evident at E:T ratios of 8:1 and 16:1 and at higher E:T ratios IL-2 secretion was too low to measure accurately. At lower effector to target ratios it appeared that IL-2 secretion was saturating from non-transduced cells. These observations were repeated in LoVo cells with two of the three donors tested against LS174T with similar results (FIGS. 3D & E).

To determine the impact of CoStAR on T-cell expansion, transduced or non-transduced T-cells were mixed with wild-type or OKT3-GFP engineered LoVo cells the number of total cells after 3 days was counted. CoStAR enhanced survival and/or proliferation of engineered T-cells in response to LoVo-OKT3 but not wild-type LoVo cells in the presence of IL-2 (FIG. 4A) and absence of IL-2 (FIG. 4B). To further investigate this phenomenon, cell proliferation analysis was performed in T-cells from two donors using proliferation dye to count the number of cell cycles each population went through over 6 days (FIGS. 4C & D). A larger proportion of CoStAR engineered cells went through 5, 6 or 7 proliferation cycles over 6 days compared to non-engineered cells in response to LoVo− OKT3, whereas CoStAR transduced and non-transduced cells went through an average of approximately 2 cycles over the same duration in response to wild-type LoVo.

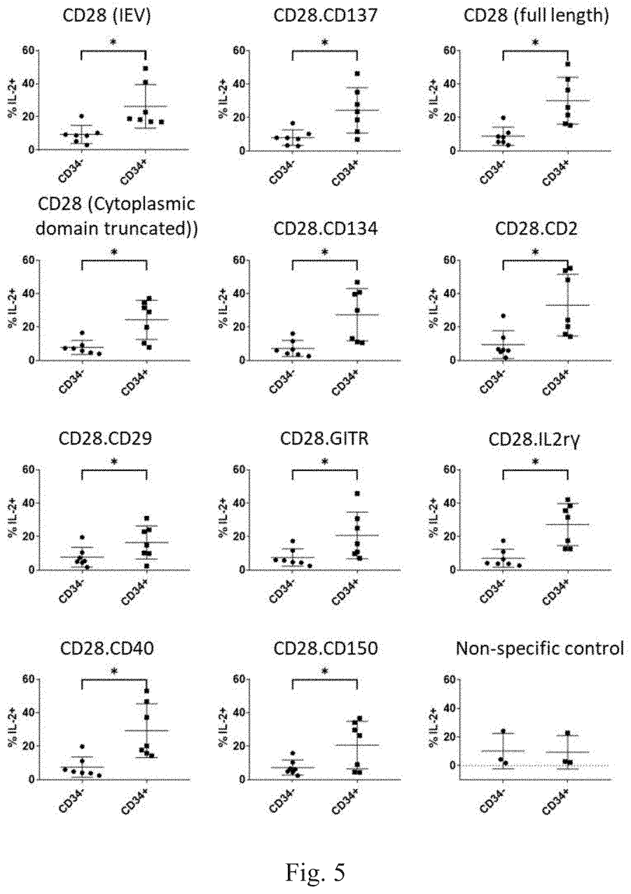

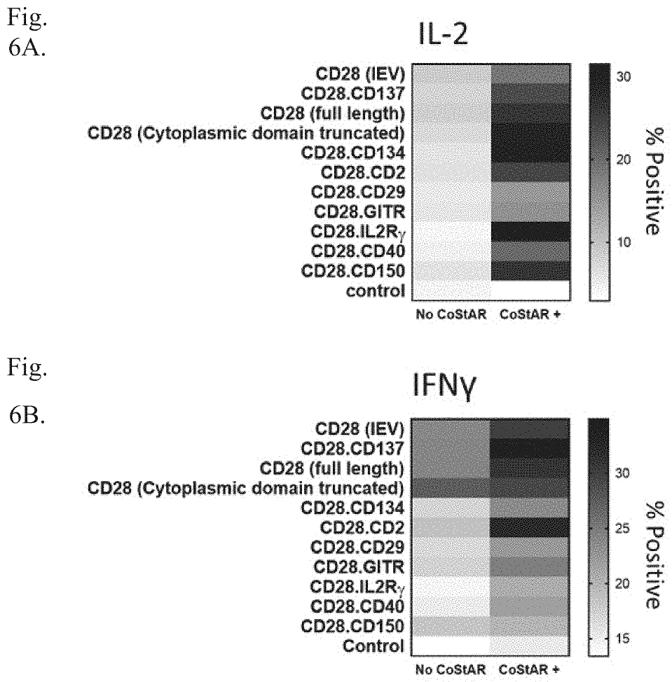

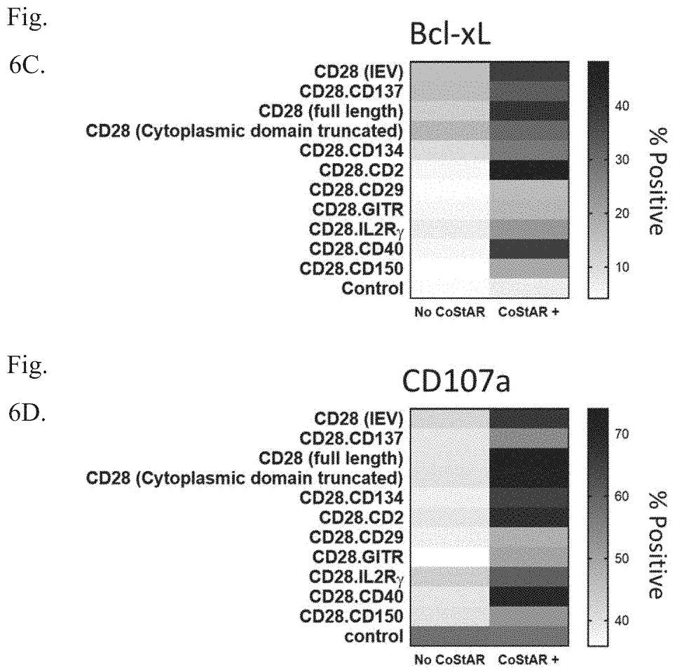

A variety of fusion receptors consisting of CD28 fused to an N-terminal additional costimulatory domain were generated. Costimulatory domains obtained from: CD137, CD2, CD29, CD134, CD150, CD40, GITR and the signalling domain from the IL-2 receptor γ-chain (IL2RT) were chosen. A receptor as close to that used in previous studies of inducible costimulation was included. This receptor designated CD28(IEV) is truncated such that the C-terminal motif of CD28 is the amino acid triad ‘IEV’. Sequences were generated de novo by Genewiz and cloned into a lentiviral vector under an EF1α promoter along with a CD34 marker gene separated from the fusion CoStAR by a 2A self-cleaving peptide. Primary CD8+ T-cells were isolated using EasySep beads (StemCell Technologies) and activated with anti-CD3/anti-CD28 activation/expansion Dynabeads before addition of lentiviral particles. Following a short expansion period the cells were mixed with LoVo or LoVo-OKT3 cells, with the inclusion of anti-CD107a antibodies and brefeldin and monensin, and following a 16 hour incubation were fixed and stained with antibodies to the marker gene (CD34) as well as antibodies to IL-2, IFNγ and bcl-xL. Analysis was performed using a MACSQuant analyser and MACSQuantify software. FIG. 5 shows the IL-2 response from CD34− (CoStAR non-transduced) and CD34+ (CoStAR transduced). Statistical analysis demonstrated that all receptors tested induced a significant increase in the proportion of cells producing IL-2 when harbouring the variant CoStAR receptors. Three other read outs were concurrently measured: IFNγ, a cytokine released under normal signal 1 conditions but enhanced by costimulation; CD107a, a marker of degranulation; and bcl-xL, an antiapoptotic protein upregulated by costimulation. Engagement of CoStAR enhanced all the effector functions analysed to varying degrees. CD28.CD2 and CD28.CD40 fusions receptors appeared to elicit the most robust response of all the receptors tested (See FIG. 6)

### Example 2

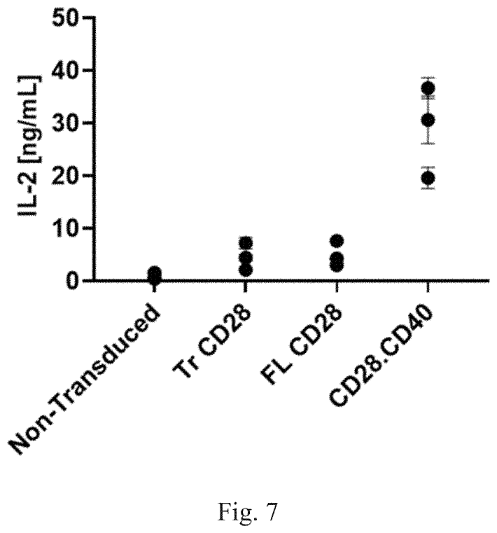

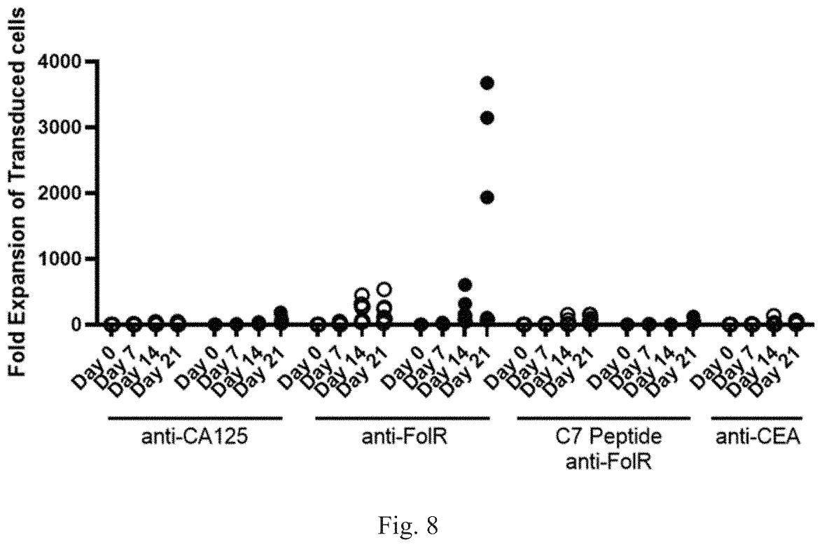

The effect of CD28 and CD28.CD40 based CoStARs on population based cytokine secretion was compared. Primary T-cells from three donors were transduced with either the CD28(IEV) truncated CoStAR, full length CD28 CoStAR or CD28.CD40 CoStAR (having the full length CD28 as shown in SEQ ID NO:439, but lacking the N terminal N and K residues) or left non-transduced. T-cells were enriched for CoStAR expression using the CD34 marker gene, and following expansion cells were mixed with LoVo-OKT3 cells and IL-2 secretion analysed by ELISA (See FIG. 7). Non-transduced cells on average produced 0.80 ng/ml IL-2, with CD28(IEV) and full length CD28 CoStAR producing 4.6 and 5.0 ng/ml IL-2 respectively. However CD28.CD40 induced 29.0 ng/ml IL-2 on average across three donors thus demonstrating a clear benefit to incorporating CD40 into the basic CD28-based CoStAR.

Next the effect of CoStAR on T-cell expansion was analysed. T-cells from seven donors were transduced with either CD28 or CD28.CD40 CoStARs with either an anti-CA125 (196-14) or anti-Folate receptor (MOV-19) scFv, or an anti-Folate receptor peptide (C7) antigen binding domain. Additional cells were transduced with a CD28 CoStAR harboring an anti-CEA scFv as a mismatched control. Cells were then mixed with CA125+/Folate receptor+/CEA− cell line OvCAR3 engineered to express a membrane bound OKT3 (OvCAR-OKT3). T-cell counts were made after 7, 14 and 21 days, and fresh OvCAR− OKT3 added at days 7, and 14. Limited expansion of cells harbouring the anti-CA125 scFv was observed (mean fold expansion: CD28: 15.1; CD28.CD40: 69.1), however cells targeting Folate receptor with an scFv did expand in both the CD28 and CD28.CD40 cohorts (mean fold expansion: CD28: 186.7; CD28.CD40: 1295.0). More limited expansion was seen when the C7 peptide was used to target the Folate receptor (mean fold expansion: CD28: 71.5; CD28.CD40: 28.0). The control CEA targeting receptor demonstrated limited expansion (mean fold expansion: 28.0).

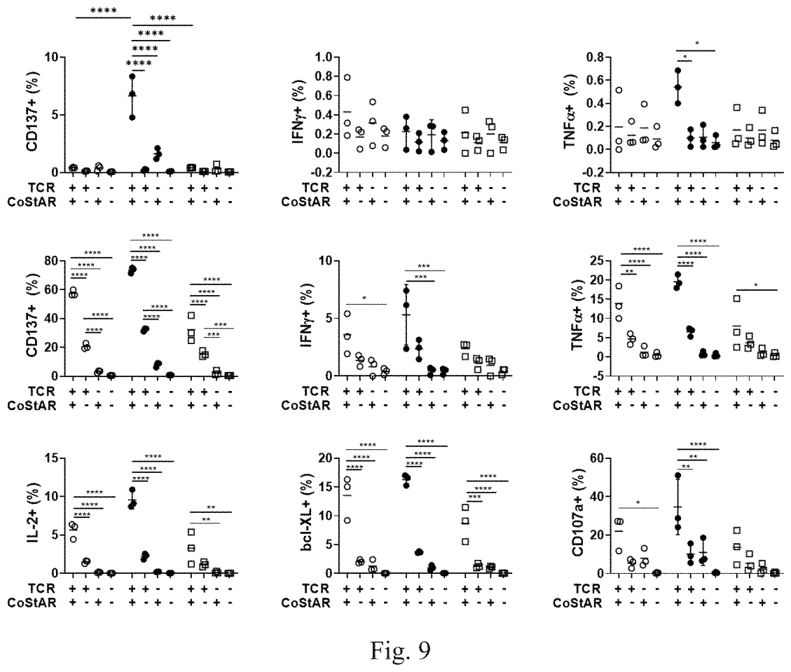

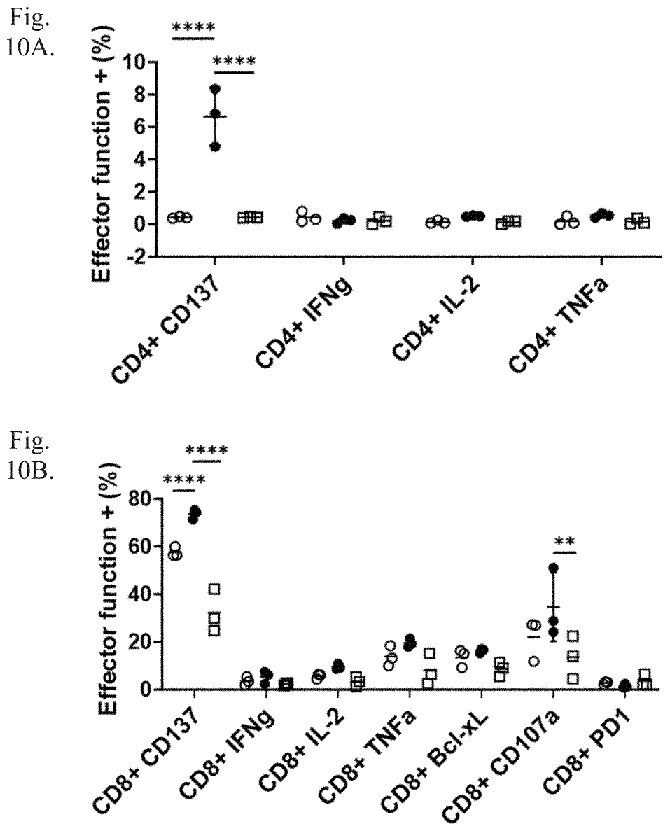

To better understand the synergy of signal 1 and signal 2 T-cells were engineered with a murine constant domain modified TCR which recognizes a CEA peptide (691-699) in the context of HLA-A*02 as well as the CD28 or CD28.CD40 CoStAR targeted towards cell surface CEA protein. As a control cells were also transduced with a CA125 specific CD28 CoStAR. The T-cells were mixed with HLA-A*02+/CEA+H508 cells and cytokine production analysed by intracellular flow cytometry staining. Flow cytometric gating was performed using antibodies directed towards the murine TCRβ constant domain (marks the TCR engineered cells) as well as the DYKDDDDK (SEQ ID NO:449) epitope tag (marks the CoStAR engineered cells). Thus it was possible to analyse the TCR−/CoStAR−, TCR+/CoStAR−, TCR−/CoStAR+ and TCR+/CoStAR+ cells in each coculture well. Cytokine production was then plotted in each subpopulation in either the CD4+ or CD8+ T-cells (FIG. 9). In CD4+ cells CD28.CD40 CoStAR enhanced CD137 and TNFα production above TCR stimulation alone, however the TCR response in CD4+ cells was poor due to the dependency of the TCR on CD8. In CD8+ cells there was more robust effector activity with IL-2 and CD107a in particular showing a stronger induction in the CD28.CD40 CoStAR groups. To better compare the receptors the effector activity in just the TCR+/CoStAR+ groups was plotted in CD4+ and CD8+ cells (FIG. 10). In CD4+ cells induction of CD137 was significantly enhanced by CD28.CD40 compared to either CEA or mismatched targeting CD28 CoStAR. In CD8+ cells CD137 induction was significantly increased compared to either CEA or mismatched targeting CD28 CoStAR, whereas CD107a induction was increased compared to the control CoStAR. Thus CD28.CD40 shows enhanced effector activity across a broad range of models and effector activities.

### Example 3

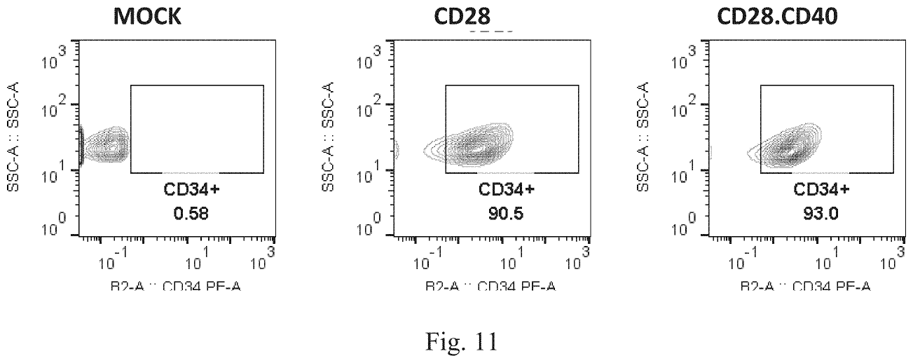

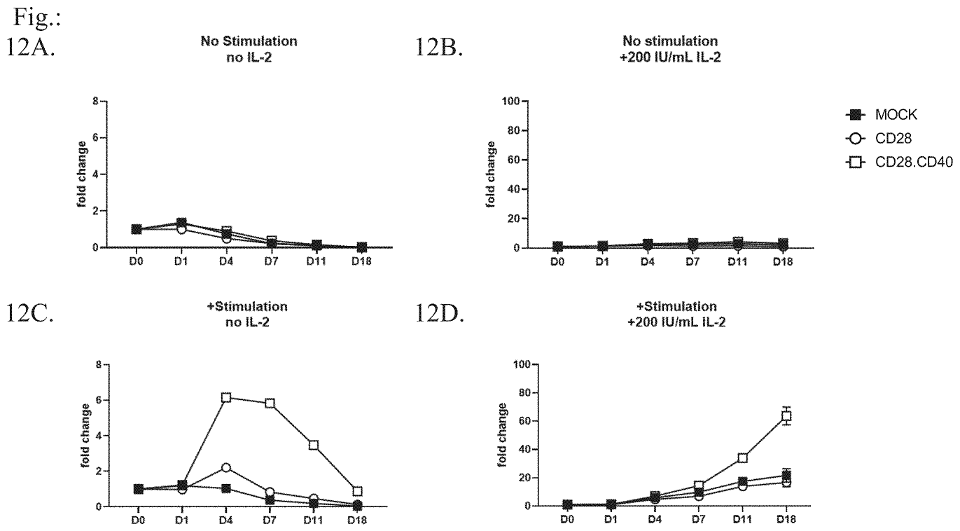

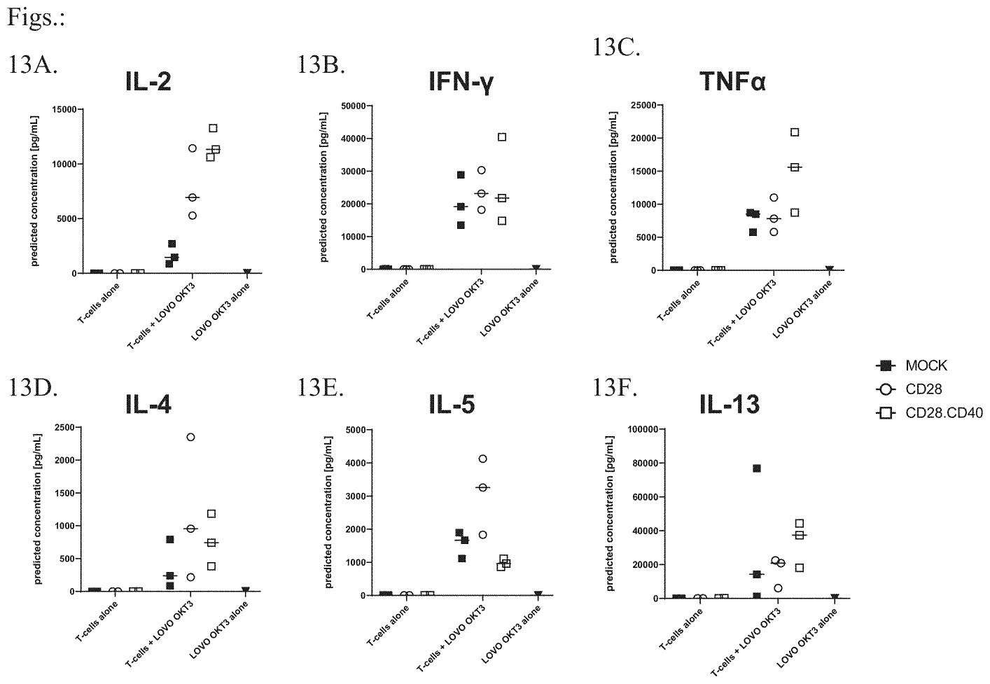

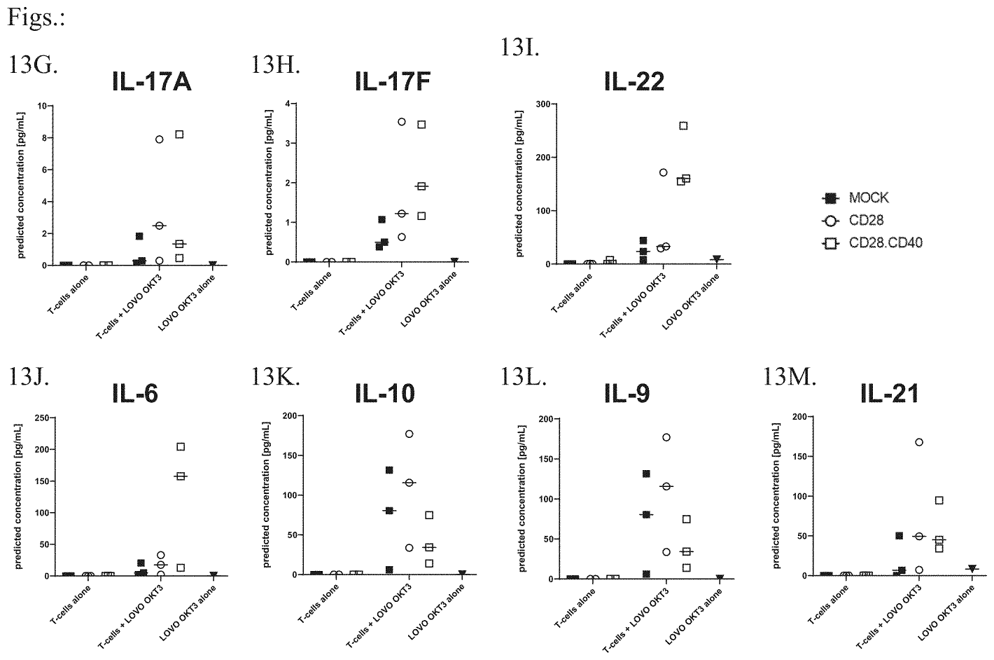

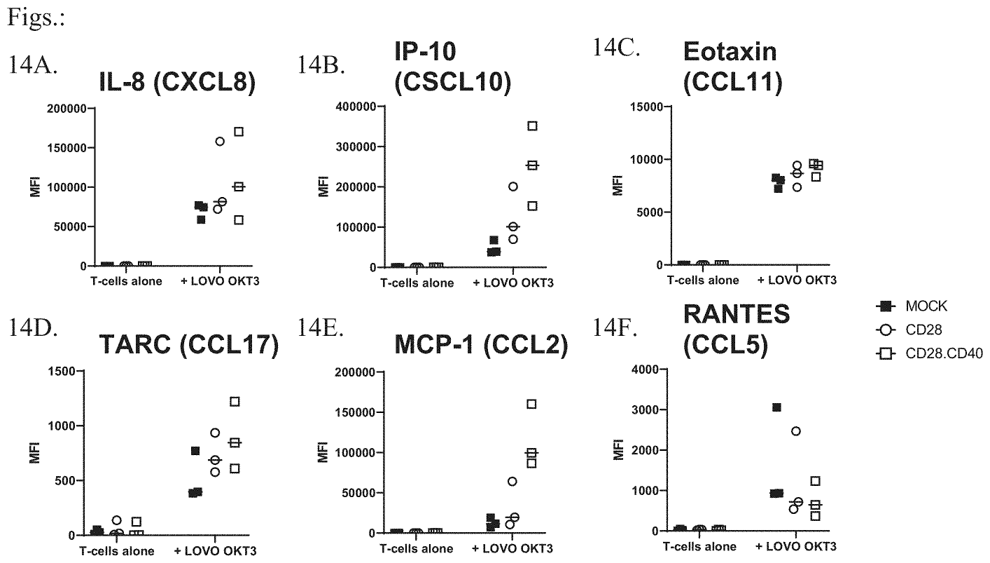

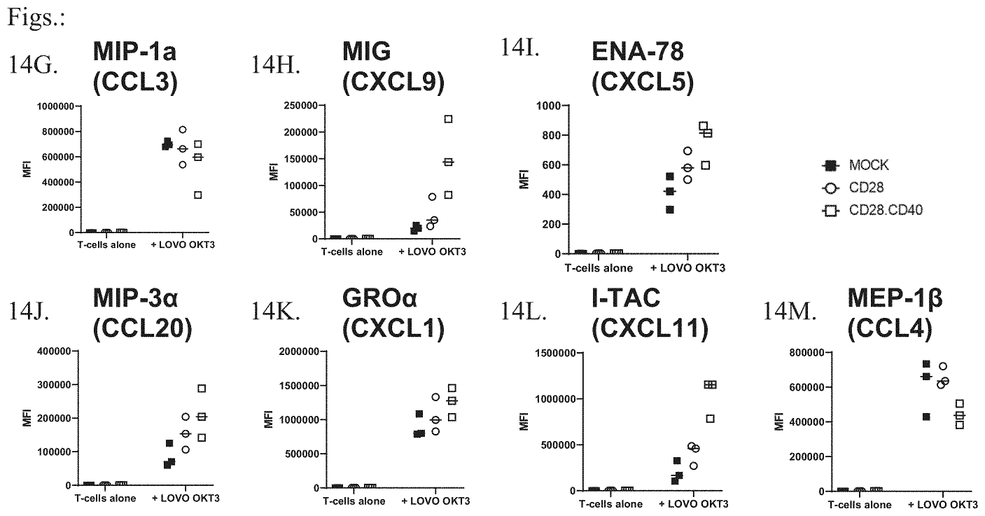

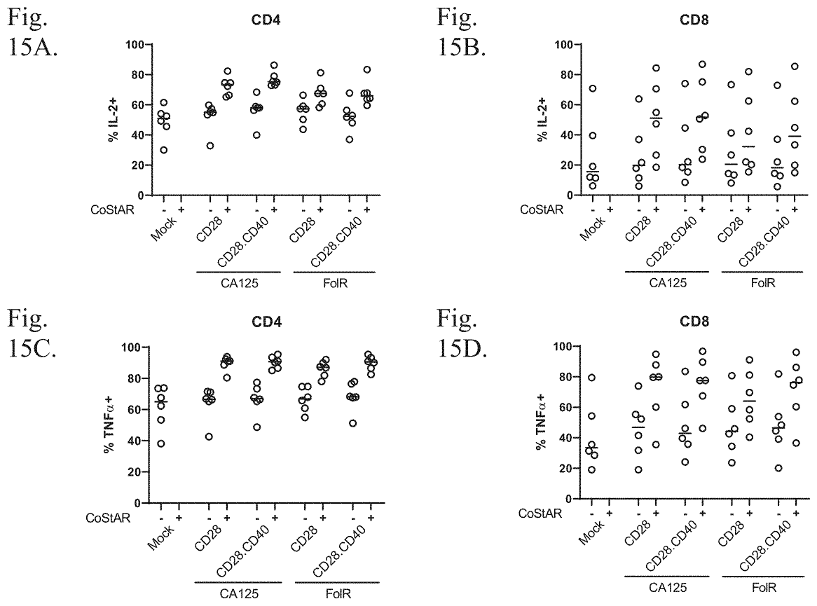

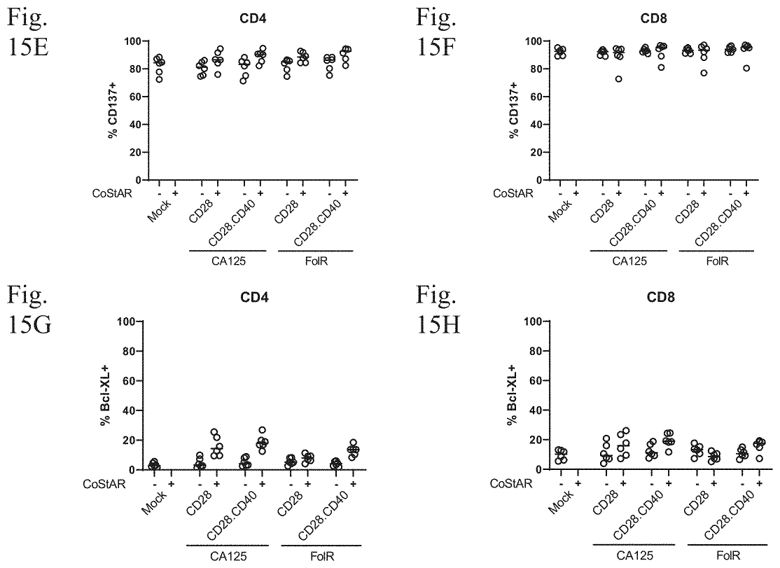

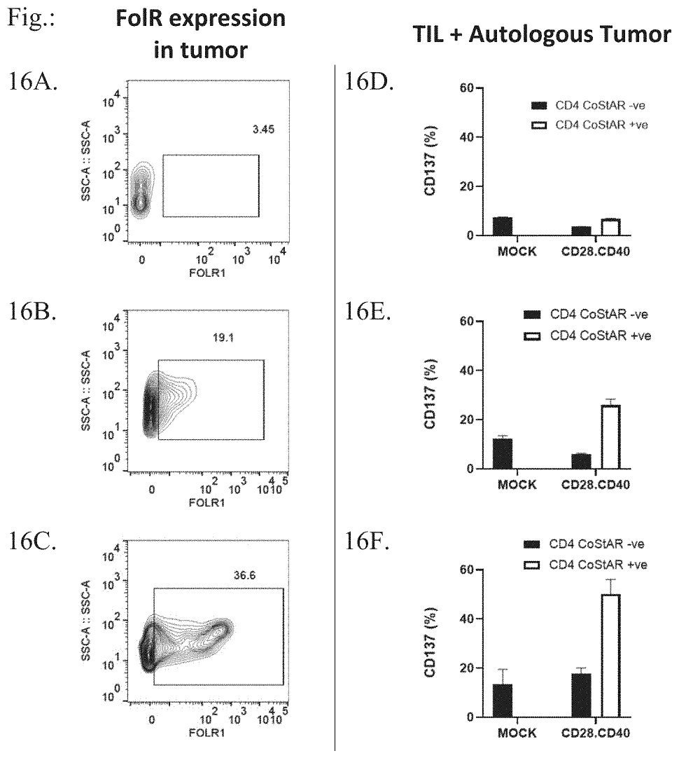

To evaluate costimulation by CD40 bearing CoStARs, primary human T-cells were mock transduced or transduced with MFE23.CD28 or MFE23.CD28.CD40 CoStAR, each harbouring a CD34 marker gene separated by a 2A cleavage peptide. MFE23 is a single chain Fv antibody that has a high affinity for carcinoembryonic antigen (CEA). Following in vitro culture cells were enriched for CD34 using MACS™ paramagnetic selection reagents (Miltenyi Biotech) and then the cells expanded in number using irradiated feeder cells. MFE23.CD28 CoStAR strongly mediated expansion of CD34⁺ T cells, and MFE23.CD28.CD40 CoStAR further enhanced expansion (FIG. 11).

To evaluate costimulatory activity and persistence, T cells mock transduced or transfected with MFE23.CD28 or MFE23.CD28.CD40 were cocultured with LoVo-OKT3 cells at an 8:1 effector:target ratio in the presence (200 IU/ml) or absence of exogenous IL-2. At days 1, 4, 7, 11 and 18 cells were taken and the number of viable T-cells enumerated by using anti-CD2 reagents on a MACSQuant flow cytometer. In the absence of stimulation by tumor and IL-2, cells declined in number as would be expected (FIG. 12A). In the absence of stimulation but presence of IL-2 there was a more apparent survival of the cells, but no specific growth (FIG. 12B). In the presence of tumor, but absence of IL-2 mock cells did not show specific survival. MFE23.CD28 CoStAR mediated an apparent doubling in expansion over the first four days followed by decline. MFE23.CD28.CD40 mediated a greater expansion up to day 7 followed by a steady decline (FIG. 12C). Under the same conditions but in the presence of IL-2 both mock and MFE23.CD28 transduced cells demonstrated a 20-fold expansion over 18 days, whereas MFE23.CD28.CD40 cells expanded by over 60-fold (FIG. 12D). Thus CD28.CD40 based receptors demonstrated superior expansion and survival under conditions of stimulation both in the presence and absence of exogenous IL-2.

Mock transduced and T cells transduced with MFE23.CD28 or MFE23.CD28.CD40 CoStARs were then tested for cytokine production. Bead array analysis was performed on supernatants obtained from T-cell/tumour cocultures. Engineered T-cells were incubated at a 1:1 effector:target ratio with LoVo-OKT3 cells for 24 hours and supernatant collected. Conditioned supernatant was also collected from an equal number of T-cells alone, or LoVo-OKT3 cells alone. Production of IL-2, IFN-γ, TNFα, IL-4, IL-5, IL-13, IL-17A, IL-17F, IL-22, IL-6, IL-10, IL-9, and IL-21 was analysed using a Legendplex™ Human TH1/TH2 cytokine panel (Biolegend) (FIG. 13A-13M). Cytokines were either very low or undetectable in media from T-cells or tumour alone. However when cocultured with tumour cytokine production was enhanced. MFE23.CD28 enhanced production of IL-2, IL-5, IL-17A/17F, IL-10, IL-9 and IL-21 compared to mock. However, MFE23.CD28.CD40 also enhanced production of TNFα, IL-13 and IL-22. MFE23.CD28.CD40 also enhanced the production of a number of cytokines greater than that elicited by MFE23.CD28 (IL-2, IL-9 and IL-17F), but also reduced the production of some cytokines below the levels seen with MFE23.CD28 (IL-5 and IL-10). Together this data demonstrates that addition of CD40 to CD28-based Costimulatory receptors enhances and/or modulates their specific activity with respect to chemokine production.

Mock transduced and T cells transduced with MFE23.CD28 or MFE23.CD28.CD40 CoStARs were further tested for chemokine production. Production of IL-8 (CXCL8), IP-10 (CSCL10), Eotaxin (CCL11), TARC (CCL17), MCP-1 (CCL2), RANTES (CCL5), MIP-1a (CCL3), MIG (CXCL9), ENA-78 (CXCL5), MIP-3a (CCL20), GROα (CXCL1), I-TAC (CXCL11), and MEP-10 (CCL4) was analysed using a Legendplex™ Human Pro inflammatory chemokine panel. (FIG. 14A-14M). Chemokines were either very low or undetectable in media from T-cells alone. However when cocultured with tumor, chemokine production was enhanced. MFE23.CD28 enhanced production of CXCL5, CXCL10, CXCL11, CCL17 and CCL20 compared to mock. However, MFE23.CD28.CD40 also enhanced production of CCL2, CXCL1 and CXCL9. MFE23.CD28.CD40 also further enhanced the production of certain cytokines to a greater amount than that elicited by MFE23.CD28 (CXCL1, CXCL9, CXCL10, CXCL11, CCL17, CCL2, CXCL9, CCL5 and CCL20), while reducing the production of some cytokines below the levels seen with MFE23.CD28 (CCL4). Together this data demonstrates that addition of CD40 to CD28-based Costimulatory receptors enhances and/or modulates their specific activity with respect to chemokine production.

CoStARs were tested for functional activity against cancer targets. Cells were transduced with CD28 or CD28.CD40 CoStARs engineered with an scFv binding domain specific for FolR or CA125 (scFv MOV19 and scFv 196-14 respectively). Human folate receptor alpha (FolR) represents a suitable target for a number of tumours including ovarian, head and neck, renal and lung and CA125 represents an alternative target for ovarian cancer. Primary human T-cells from six healthy donors were engineered with either 196-14.CD28, 196-14.CD28.CD40, MOV19.CD28 or MOV19.CD28.CD40 receptors, all harbouring a DYKDDDDK epitope tag for detection. Transduced cells were mixed with FolR+/CA125+ OvCAR-OKT3 cells before analysis of effector activity using intracellular staining in the epitope tag positive and negative populations. Specific enhancement of effector activity determined by production of IL-2 (FIGS. 15A and 15B), TNFα (FIGS. 15C and 15D), CD137 (FIGS. 15E and 15F), and BCL-xL (FIGS. 15G and 15H) was observed in CD28 and CD28.CD40 engineered cells compared to mock transduce cells in response to both CA125 and FolR, although specific BCL-xL induction by MOV19.CD28 was not substantial as compared to MOV19.CD28.CD40.

Mock transduced TILs or TILs engineered with MOV19.CD28.CD40 CoStAR were evaluated for expansion and CD137 production stimulated by patient matched tumour digest (FIG. 16). Three donor tumours were tested which displayed varying levels of FolR on the digest, ranging from negative (6A), low expression (6B) to high expression (6C). Mock and CoStAR negative TIL in the CoStAR engineered populations of TIL matched for the FolR negative digest demonstrated similar levels of CD137 upregulation following tumour coculture which was not enhanced by the presence of CoStAR (FIG. 16D). In the TIL exposed to FolR low expressing digest there was an enhancement in activity in the CoStAR+ cells compared to CoStAR−, with CD137 expression increasing from <10% to >20% (FIG. 16E). In the TIL exposed to FolR high expressing tumour digest there was an increase in activity from around 20% in the CoStAR− population, up to approximately 50% in the CoStAR+ population (FIG. 16F).

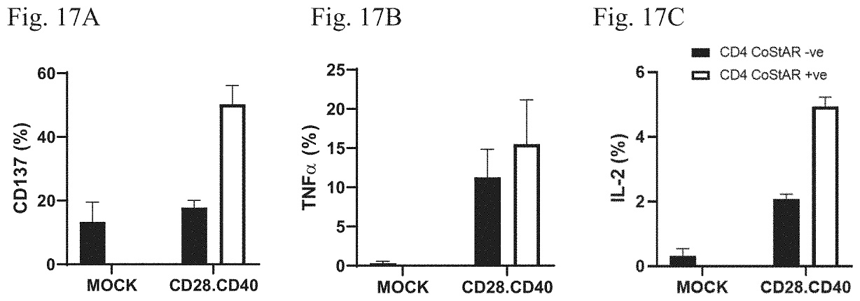

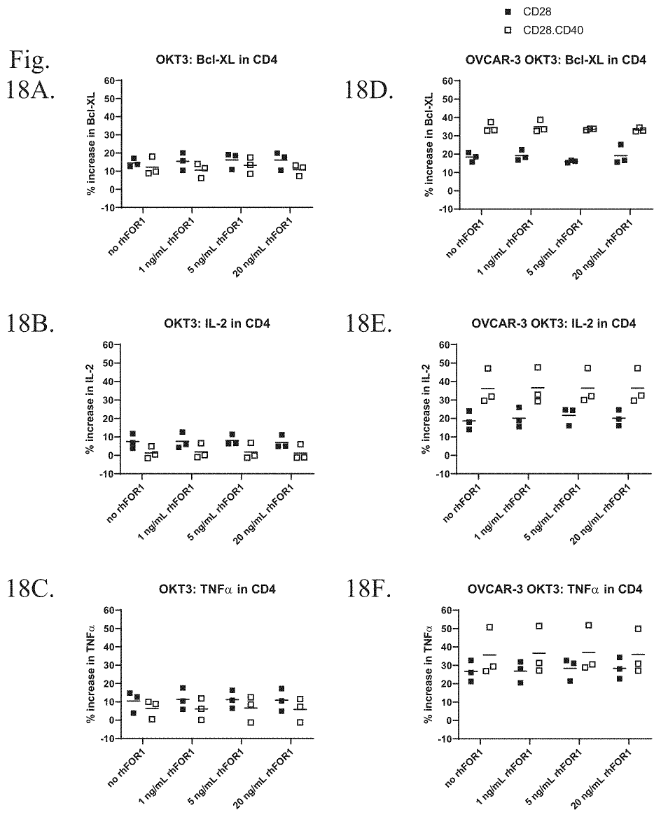

A FolR targeting CoStAR was examined for enhancement of effector functions. MOV19.CD28.CD40 enhanced CD137 expression from ˜20% to ˜50% (FIG. 17A), TNFα production from 10% to 15% (FIG. 17B) and IL-2 production from 2% to 5% (FIG. 17C) in response to FolR+ tumour digest.

CoStAR mediated stimulation by soluble ligand was also examined. T-cells from three healthy donors were engineered with MOV19.CD28 or MOV19.CD28.CD40 CoStAR and activated with either immobilised OKT3, providing stimulation in the absence of FolR, or with OvCAR-OKT3, to provide TCR and CoStAR activity. Bcl-XL activity was increased from between 10 and 20% across the three donors following OKT3 stimulation (FIG. 18A) whereas IL-2 was increased between 0 and 12% (FIG. 18B) and TNFα increased between 0 and 20% (FIG. 18C). The presence of exogenous soluble FolR did not enhance any of these particular effector functions. In the presence of OvCAR-OKT3 Bcl-XL induction was enhanced by ˜20% in CD28 CoStAR and by ˜35% in CD28.CD40 CoStAR (FIG. 18D), IL-2 induction was enhanced by ˜20% in CD28 CoStAR and 30-50% in CD28.CD40 CoStAR (FIG. 18E) and TNFα production was enhanced by 20-30% in CD28 CoStAR and 25-50% in CD28.CD40 CoStAR (FIG. 18F). Exogenous soluble FolR did not have an inhibitory effect on any of these effector functions.

### Example 4

Materials and Methods

Construct design—The MFE23, MOV19 and 196-14 CoStAR constructs include an MFE23 (CEA specific), MOV19 (Folate receptor a specific) or 196-14 (CA125 specific) derived single chain antibody fragment nucleotide sequence with an oncostatin M1 leader sequence fused to a costimulatory domain. The costimulatory domains contain an extracellular spacer region and transmembrane domain derived from human CD8 or CD28 and a signalling domain of either CD28, CD2 or CD137 and/or wild-type or mutant CD40 variants. Some CoStARs detailed herein comprise a human PD1 extracellular domain fused to CD28 and CD40. Receptors were cloned with a P2A cleavage sequence and a truncated form of human CD34 to permit detection of transduced cells. The CoStAR nucleotide sequence was codon optimised and gene synthesised by Genewiz Inc. The constructs were cloned into a third generation lentiviral vector.

Peripheral blood mononuclear cells were isolated from normal healthy donors before activation for 24 hours with T-cell activation and expansion beads (Invitrogen) according to the manufacturer's instructions before addition of lentiviral supernatants.

Cell transduction was assessed 96 hours post infection using CEA.hFc protein (R&D Systems) and anti-hFc-PE secondary, plus anti-CD34-APC or by anti-CD34-PE antibodies alone. Cells were then expanded further using ×10 donor mismatched irradiated PBMC feeders at a 1:20-1:200 ratio in RPMI+10% FCS with the addition of 30 ng/ml OKT3 and 200 IU/ml IL-2. After 14 days the cells were stained as previous and stored ready for assay.

Functionality assays were performed by mixing CoStAR positive or negative cells with wild-type or OKT3 engineered CEA-Positive LoVo cells. Briefly, T-cells were mixed with LoVo cells at varying ratios in 96-well plates. For flow analysis cocultures were incubated with Brefeldin and monensin and anti-CD107a antibodies for 16 hours following which cells were stained with Fixable Viability Dye ef450 (eBiosciences), fixed with 4% paraformaldehyde and then permeabilised using Fix/Perm wash buffer (BD Biosciences). Cells were then stained with anti-CD34 or anti DYKDDDDK antibodies to differentiate between the CoStAR+ and CoStAR-populations, anti-IL-2, anti-TNFα and anti-IFNγ antibodies (Biolegend). For soluble analyte analysis supernatants were collected for analysis by ELISA, cytokine bead array (LEGENDPLEX™ Human Th Cytokine Panel (12-plex)) or chemokine bead array (LEGENDPLEX™ Human Proinflammatory Chemokine Panel (13-plex).

Proliferation assays were performed by mixing T-cells and tumour cells at an 8:1 effector:target ratio in complete T-cell media (TCM: RPMI supplemented with 10% FCS, 0.01 M HEPES and 1% Penicillin/streptomycin, 50 mM β-mercaptoethanol) in the presence or absence of IL-2. Cell counts were made at indicated time points and fresh tumour cells were added in restimulation assays at a final E:T of 8:1. Cell counts for proliferation assays were performed by taking cells from the wells and staining with anti-CD2 PerCP eFluor710 antibody (eBioscience, UK) for 20 min in the dark, followed by DRAQ7 staining and counts made using a MACSQuant analyser.

### Example 5

To determine which receptor signaling motifs contribute to CD40 enhancement of CoStAR activity, variant receptors were generated harbouring mutations in the TRAF6 binding motif (MFE23.CD28.CD40 (PQEINF mutated to AQAINF)), the TRAF2 binding motif (MFE23.CD28.CD40 (SVQE mutated to AVQA)) or the TRAF1/2/3/5 binding motif (MFE23.CD28.CD40 (PVQET mutated to AVAEA)). Primary human T-cells isolated from three separate healthy donors were transduced with the indicated CoStARs and cells enriched for CoStAR expression via CD34. Cocultures were set up with LoVo-OKT3 cells and supernatants collected for cytokine analysis.

IL2 production by non-transduced cells was very low, but elevated in cells expressing the wild-type MFE23.CD28.CD40 CoStAR. IL2 production was not largely affected by mutations to the SVQE, PQEINF or Q263A mutation. However, mutation to the PVQET motif had a dramatic effect on IL2 production by T-cells.

Concurrently proliferation of engineered T-cells was assessed in response to LoVo-OKT3 cells over 2 rounds of stimulation with LoVo-OKT3 cells. Non-transduced cells did not survive even one round of stimulation, whereas cells expressing the wild-type receptor underwent a 10-fold expansion over 2 rounds of stimulation over 14-days. Cells harboring either a SVQE-AVQA mutation, or PQEINF-AQAINF mutation displayed a reduced capacity to undergo proliferation, this was further exacerbated in cells harboring a Q263A mutation. However, cells harboring a PVQET-AVAEA mutation in the TRAF2/3 binding region displayed a profound inability to proliferate over 2 rounds of stimulation.

### Example 6

Design of CoStARs that bind to CEA—The CoStAR consists of a CEA specific MFE23, humanised (hu) MFE23, CEA6, BW431/26 or huT84.66 derived single chain antibody fragment nucleotide sequence with an oncostatin M1, CD8α, CD2, IL-2, GM-CSF or hIgGκ VIII leader sequence. Each CoStAR has an extracellular spacer domain derived from CD8 or CD28 or truncated CD28 and a signalling domain derived from CD28 and CD40. The constructs are cloned into pSF.Lenti (Oxford Genetics) containing an MND promoter, and separated from a truncated CD34 marker gene via a P2A cleavage sequence.

Lentiviral Production—Lentiviral production is performed using a three-plasmid packaging system (Cell Biolabs, San Diego, USA) by mixing 10 μg of each plasmid, plus 10 μg of the pSF.Lenti lentiviral plasmid containing the transgene, together in serum free RPMI containing 50 mM CaCl₂). The mixture is added dropwise to a 50% confluent monolayer of 293T cells in 75 cm2 flasks. The viral supernatants are collected at 48 and 72 h post transfection, pooled and concentrated using Lenti-X lentiviral supernatant concentration (Takara Bio Inc. Japan) solution according to the manufacturer's instructions. Lentiviral supernatants are concentrated 10-fold and used to directly infect primary human T-cells at an MOI of 3-5 in the presence of 4 μg/ml polybrene (Sigma-Aldrich, Dorset, UK). Peripheral blood mononuclear cells are isolated from normal healthy donors before activation for 24 hours with T-cell activation and expansion beads (Invitrogen) according to the manufacturer's instructions before addition of lentiviral supernatants.

Cell transduction is assessed 96 hours post infection using CEA.hFc protein and anti-hFc-PE secondary, plus anti-CD34-APC or by anti-CD34-PE antibodies alone. Cells are then expanded further using ×10 donor mismatched irradiated PBMC feeders at a 1:200 ratio in T-cell media (RPMI 1640, 10% FBS, 10 mM HEPES, 50 μM β-mercaptoethanol and 50 u/ml Penicillin/streptomycin), 200 IU/ml IL-2 and a final concentration of 30 ng/ml anti-CD3 (OKT3). After 12-14 days the cells are stained as previous and stored ready for assay.

Functionality assays are performed by mixing CoStAR positive or negative cells with wild-type or OKT3 engineered CEA positive cell lines (LoVo, HT29, SW480, H508). Briefly, T-cells are mixed with target cells at varying ratios in 96-well plates and cytokine release measured by ELISA and MSD analysis.

Cytotoxicity assays are performed using the xCELLigence RTCA SP real time cell analyser system (Acea). A program was generated to test well conductivity (cell index) of a 96-well PET E-plate every 15 min for the duration of the experiment (up to 250 hr). 50 μL T cell medium was added to the wells which were to have cell index tested and incubated at room temperature (RT) for 30 minutes. The E-plate was added to the RTCA SP device and background conductivity readings measured.

The optimal density of target cells to seed is defined as that which reaches a stable cell index between 24- and 36-hours after the beginning of the assay and does not decrease without intervention (i.e. addition of Triton-x-100 or effector cell populations) before the end of the assay. Target cells at optimal density for killing assays (cell line dependent) are counted using a quantitative method capable of dead-cell discrimination. The E-plate is removed from the device and the optimal density of live cells is added to the wells containing T cell medium at a final volume of 100 μL before incubation for 30 minutes at RT.

The E-plate us then placed back on the analyser and cell index values acquired until a stable cell index is observed, at which point the program is paused and the E-plate removed from the RTCA SP device. Treatments are added to the appropriate wells. Treatments consist of either 100 μL T cell medium (no treatment control), or the same volume containing effector cells or 0.5% Triton-x-100 (full lysis control). Effector cell counting uses a quantitative method capable of dead- and apoptotic-cell discrimination. The number of effectors to target cells varied depending on the experiment.

During data analysis, the cell index is normalized using the following equation in the RTCA software package:

NormalisedCIti=CIti/CInml_time

Where CIti=Cell index (CI) at a specific time point, and CInml_time=CI at the time point prior to addition of T cells.

Data is then further manipulated relative to the full lysis control to give % cytolysis:

% Cytolysisst=[1−(NCIst)/(AvgNCIRt)]×100

where, NCIst is the Normalised Cell Index for the sample and NCIRt is the average of Normalise Cell Index for the matching reference wells.

Repeat stimulation assays are performed by mixing 5×10⁴ CoStAR transduced or mock-transduced cells at an 8:1 E:T ration with LoVo-OKT3 cells in the absence of exogenous IL-2, in triplicate well of a 96-well U-bottom plate. T-cell counts are made on D1, 4 and 7 via flow cytometric assessment of numbers based on αCD2 gating, and fresh tumour cells added at seven day intervals. Relative expansion is assessed by splitting of wells and enumeration of fold change based upon the original seeding density with the proportion of cells removed for counting also factored in.

Results

A model system is developed to evaluate the impact of various structural components of CEA directed CoStAR receptors. To this end the signal peptide (SP), single chain antibody fragment (scFv) and extracellular spacer (ES) are assessed for their impact on expression and function.

The impact of the signal peptide on expression of the CoStAR is tested, as it is known that different signal peptides can affect expression of various recombinant proteins (REF). The MFE23.CD28.CD40 CoStAR receptors are generated with various different leader sequences (which encode the desired signal peptide) sequences. These include signal peptides derived from: Oncostatin M1 (OSM), IL2, CD2, CD8α, GMCSF and hIgGκ VIII. Each leader sequence is cloned in frame with the MFE23 scFv sequence to generate SP.MFE23.CD28.CD40.P2A.tCD34. A Jurkat cell line model is selected to investigate the relative expression of each SP modified CoStAR relative to the tCD34 marker gene. To this end Jurkat JRT3-T3.5 T-cells are incubated with lentiviral particles at an MOI of 5. Seven days post transduction the cells are stained with anti-CD34 antibodies to stain for the transduced cells, and CEA.hFc protein followed by anti-hFc secondary antibodies to identify for the CEA CoStAR. All SP modified CoStAR variants tested are found to be expressed in the CD34+ proportion of the JRT3-T3.5 cells.

Next, the impact of different CEA specific scFv in the context of CoStAR is assessed, as well as investigating different spacer domains. Six different scFvs are compared; as well as the MFE23 sequence described above (Chester et al. 1994), including an MFE23 K>Q mutant, humanised (Hu) MFE23 (Begent et al. 2003), CEA6 (Jackson et al. 1998), BW431/26 (Seenmann et al. 1991) and HuT84.66 (Yazaki et al. 2005).

Primary human T-cells are isolated from Buffy coats obtained from the NHSBT. T-cells are isolated by Ficoll-mediated isolation and T-cell negative isolation kits (StemCell Technologies). The isolated T-cells are activated with human T-cell activation and expansion beads (Invitrogen, UK). Cells are incubated with concentrated lentiviral particles, encoding CEA CoStARs containing the OSM SP, and expanded over a number of days. Cells are enriched for CoStAR expression using anti-CD34 antibodies to obtain T-cell populations greater than 90% CoStAR positive before being placed in a rapid expansion protocol (REP), with irradiated buffy coat derived PBMCs as outlined in the materials and methods.

A physiologically relevant in vitro model is employed to test the impact of CoStAR on T-cell activity. Transduced and non-transduced cells are tested against the CEA+ cell lines LoVo, H508, SW480 or HT29. The murine CEA− cell line Ba/F3 is engineered to express CEA as a control. To enable activation of the T-cells in response to the unmatched tumour lines the tumour cells are engineered to express an anti-CD3 single chain antibody fragment anchored to the cell membrane by way of a synthetic transmembrane domain and split from a GFP marker gene using an IRES element to visualise transduced cells using flow cytometry. Cell lines are also engineered to express firefly-luciferase (ffLuc) under puromycin selection to permit analysis of target cell lysis.

Non-transduced and CoStAR transduced T-cells are mixed at varying effector:target ratios with wild-type or OKT3-engineered tumour cell lines. After 24 hours coculture media is taken for IL-2 ELISA measurement. Activation dependent IL-2 secretion is observed from both CoStAR+ and CoStAR− T-cell populations from all donors in response to OKT3 engineered cells with only background IL-2 secretion seen from transduced and non-transduced T-cells in response to un-engineered tumour cells. In all donors tested, the presence of CEA CoStAR enhances effector activity (IL2, IL3, CXCL10) towards OKT3 engineered CEA+ tumour lines.

Cocultures with Ba/F3 cells demonstrate the targeted approach of the CEA CoStARs. Coculture of CEA CoStAR engineered cells with Ba/F3 or Ba/F3-CEA does not result in specific IL2 release whereas incubation with Ba/F3-OKT3 enhances IL-2 secretion. However, incubation of T-cells with Ba/F3-OKT3/CEA significantly enhances IL2 secretion compared to Ba/F3-OKT3 alone.

The impact of CoStAR on tumour cell killing is determined. Transduced or non-transduced T-cells are mixed with wild-type or OKT3-GFP engineered tumour cells and quantified residual tumour cell derived luciferase activity at defined time points. The presence of CoStAR enhances the ability of T-cells to mediate target cell lysis. This enhanced ability of CoStAR+ cells to mediate anti-tumour activity is also evident using the xCELLigence device as outlined in materials and methods.

Repeat stimulation assays are performed according to materials and methods. In brief mock or CoStAR engineered cells are mixed at an 8:1 E:T ratio with OKT3 engineered target lines and the relative expansion of T-cells enumerated at the indicated time points, with fresh tumour added at seven day intervals. All CoStARs tested mediate prolonged survival and expansion of T-cells across multiple rounds of stimulation, whereas mock transduced cells decline in number following repeat stimulations.

To evaluate CoStAR activity in TIL specimens, TIL are engineered with CEA specific CoStAR constructs. To this end tumours are digested and analysed for CEA expression using flow cytometry. Tumour digests testing positive are engineered with CEA CoStARs. Following outgrowth and rapid expansion protocol, engineered and matched non-engineered TIL are mixed with either tumour digest, or where available, matched autologous tumour lines. In all donors tested the presence of the CEA CoStAR enhances specific effector activity as measured by IFNγ and IL-2 compared to cells which are mock transduced.

### Example 7

Anti-MSLN CoStAR Expression

Anti-MSLN CoStARs comprising different scFv antigen-binding domains (Table 8) were compared for surface expression on T cells from healthy donors.

TABLE 8

| Clone | CTP224 | CTP225 | CTP226 | CTP227 | CTP228 | CTP229 |
|---|---|---|---|---|---|---|
| SEQ ID NO: | 192 | 210 | 228 | 246 | 264 | 282 |
| Signal peptide | OSM1 | | | | | |
| scFv | SS1 | M5 | HN1 | M912 | huYP218 | P4 |

| | |
|---|---|
| Linker | AAAGSGGSG |
| Spacer | CD28 EC |
| TM | CD28 |
| Intracellular | CD28 · CD40 |

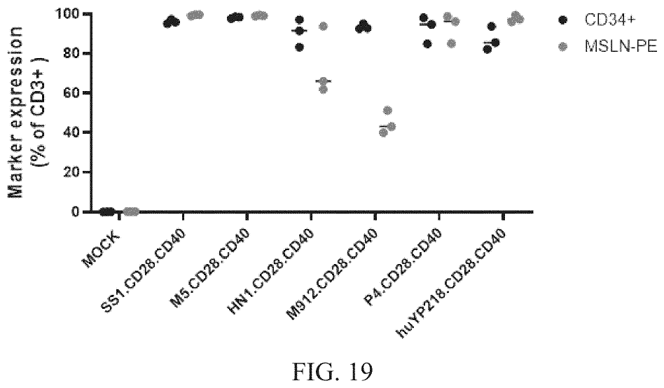

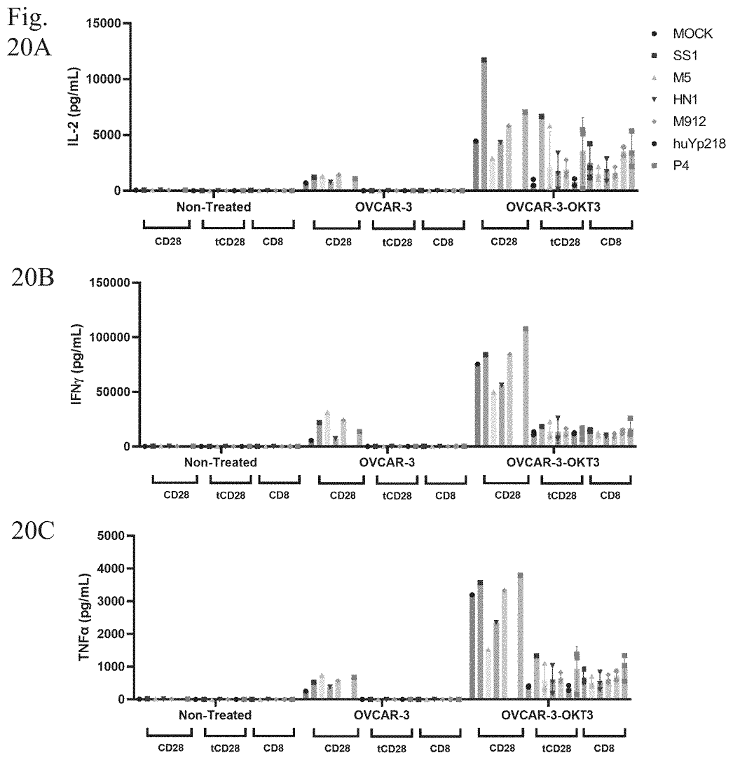

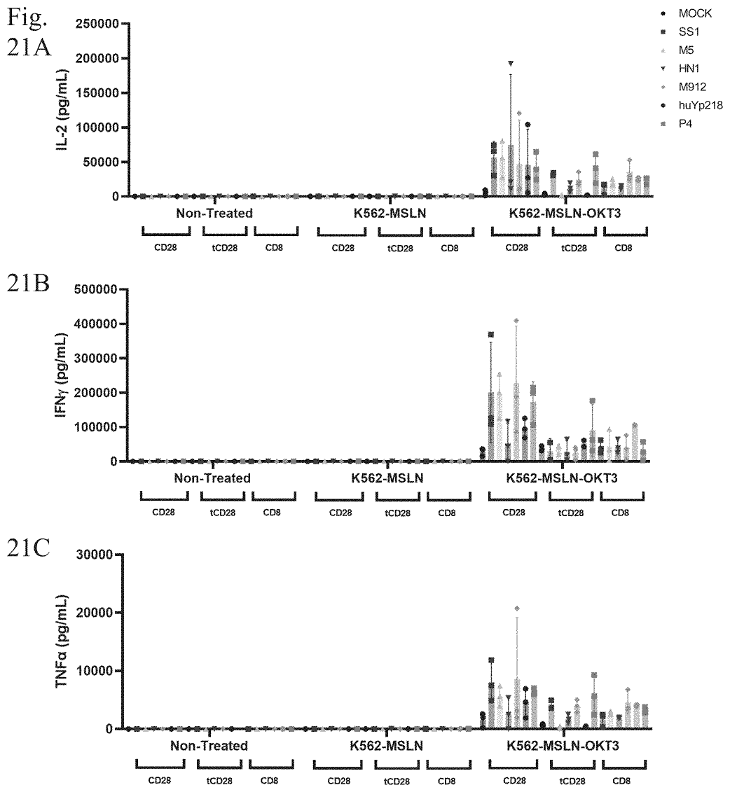

T cells from healthy donor (HD) PBMCs were lentivirus transduced, at a multiplicity of infection (MOI) 5, with six variable scFV constructs against mesothelin (MSNL) that possessed CD28.CD40 signaling domains. Non-transduced (MOCK) cells were used as controls. Cells were sorted using CD34 microbeads and underwent a rapid expansion protocol (REP) for 14 days. Following expansion, 1×10⁵ cells were assessed for transduction efficiency either via surface detection of the marker gene tCD34 or CoStAR molecule using an anti-CD34-APC (black) or anti-MSLN-PE (red) antibody, respectively. (FIG. 19). The results represent 3 biological replicates.

### Example 8

Anti-MSLN CoStAR Activity

Cytokine production was assessed in CoStAR transduced HD T cells cocultured with target cell lines. A variety of CoStARs comprising different anti-MSLN binding domains, spacers, or transmembrane domains (Table 8, Table 9, Table 10) were tested.

TABLE 9

| Clone | CTP248 | CTP249 | CTP250 | CTP251 | CTP252 | CTP253 |
|---|---|---|---|---|---|---|
| SEQ ID NO: | 300 | 306 | 312 | 318 | 324 | 330 |
| Signal peptide | OSM1 | | | | | |
| scFv | SS1 | M5 | HN1 | M912 | huYP218 | P4 |

| | |
|---|---|
| Linker | AAAGSGGSG |
| Spacer | Truncated CD28 EC |
| TM | CD28 |
| Intracellular | CD28 · CD40 |

TABLE 10

| Clone | CTP236 | CTP237 | CTP238 | CTP239 | CTP240 | CTP241 |
|---|---|---|---|---|---|---|
| SEQ ID NO: | 198 | 216 | 234 | 252 | 270 | 288 |
| Signal peptide | OSM1 | | | | | |
| scFv | SS1 | M5 | HN1 | M912 | huYP218 | P4 |

| | |
|---|---|
| Linker | AAAGSGGSG |
| Spacer | CD8 EC |
| TM | CD8 |
| Intracellular | CD28 · CD40 |

Nontransduced (MOCK) and anti-MSNL CoStAR transduced HD T cells were cocultured with engineered OVCAR3 target cell lines at an effector to target (E:T) ratio of 8:1 (1×10⁵: 1.25×10⁴) for 24 hours and MSD immunoassay was performed to evaluate the concentration of cytokines secreted. Cytokine concentrations were determined for IL-2 (FIG. 20A) IFNγ (FIG. 20B) and TNFα (FIG. 20C) following cocultures with OVCAR-3 or OVCAR3-OKT3 cell lines. Non-treated T cells were used as a control. The results represent 1-3 biological replicates with 3 technical replicates each.

Nontransduced (MOCK) and anti-MSNL CoStAR transduced HD T cells were cocultured with engineered K562 target cell lines at an effector to target (E:T) ratio of 8:1 (1×10⁵: 1.25×10⁴) for 24 hours and MSD immunoassay was performed to evaluate the concentration of cytokines secreted. Cytokine concentrations were determined for IL-2 (FIG. 21A) IFNγ (FIG. 21B) and TNFα (FIG. 21C) following cocultures with K562-MSNL or K562-MSNL-OKT3 cell lines. Non-treated T cells were used as a control. The results represent 1-3 biological replicates with 3 technical replicates each.

Anti-CEA CoStAR Expression

Anti-CEA CoStAR expression was evaluated for anti-CEA CoStARs comprising differing signal peptides (Table 11) or scFv antigen binding domains (Table 12).

TABLE 11

| Clone | CTP194 | CTP255 | CTP256 | CTP257 | CTP258 | CTP259 |
|---|---|---|---|---|---|---|
| SEQ ID NO: | 42 | 43 | 44 | 45 | 46 | 47 |
| Signal peptide | OSM1 | CD8 | CD2 | IL2 | GMCSF | hIgGK |

| | |
|---|---|
| scFv | MFE23 |
| Linker | AAAGSGGSG |
| Spacer | CD28 EC |
| TM | CD28 |
| Intracellular | CD28 · CD40 |

TABLE 12

| Clone | CTP194 | CTP219 | CTP220 | CTP221 | CTP222 | CTP223 |
|---|---|---|---|---|---|---|
| SEQ ID | 42 | 60 | 78 | 96 | 114 | 132 |
| NO: | | | | | | |
| Signal | OSM1 | | | | | |
| peptide | | | | | | |
| scFv | MFE23 | MFE23 | hMFE23 | CEA6 | BW431/26 | hT84.66 |
| | | (Q > K) | | | | |

| | |
|---|---|
| Linker | AAAGSGGSG |
| Spacer | CD28 EC |
| TM | CD28 |
| Intracellular | CD28 · CD40 |

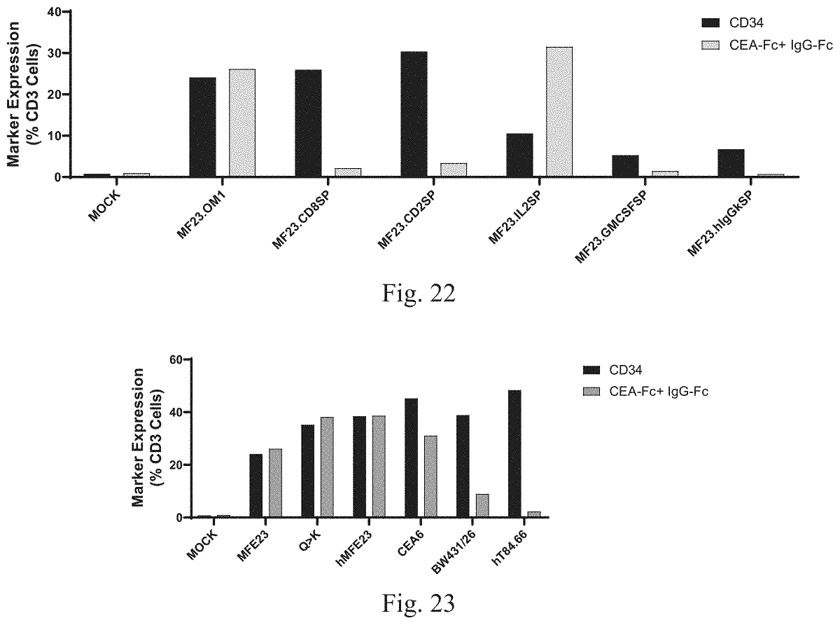

To examine signal peptide variants, T cells from healthy donor PBMCs were lentivirus transduced at a multiplicity of infection (MOI) 5, with the MFE23 scFV constructs against the carcinoembryonic antigen 5 (CEA) that possessed CD28.CD40 domains. The constructs had variations in the signal peptide and non-transduced (MOCK) cells were used as controls. Cells were sorted using CD34 microbeads and underwent a rapid expansion protocol (REP) for 14 days. Following expansion, 1×10⁵ cells were assessed for transduction efficiency (FIG. 22) either via surface detection of the marker gene tCD34 or CoStAR molecule using an anti-CD34-APC (black bars) or using a primary rhCEACAM5-Fc antibody with a secondary anti-IgG-Fc-PE (grey bars) antibody, respectively.

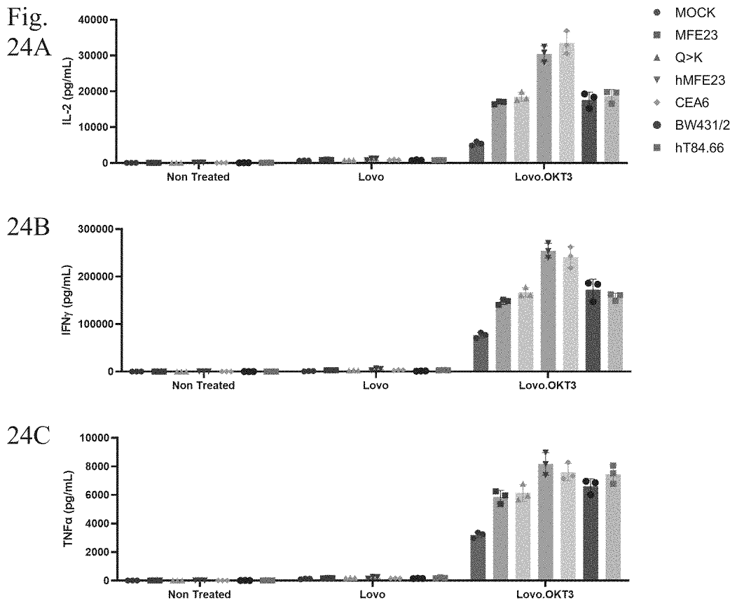

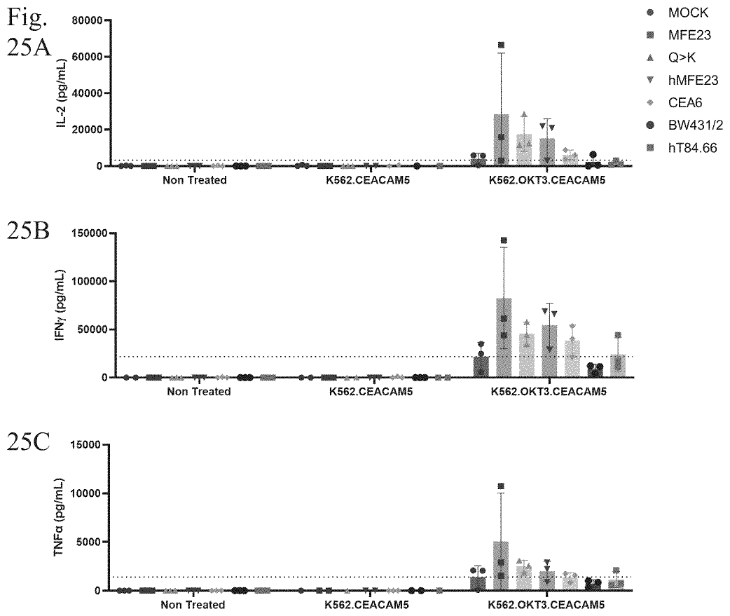

T cells from healthy donor PBMCs were also lentivirus transduced, at a multiplicity of infection (MOI) 5, with variable scFV constructs against the carcinoembryonic antigen 5 (CEA) that possessed CD28.CD40 domains. As above, non-transduced (MOCK) cells were used as controls. Cells were sorted using CD34 microbeads and underwent a rapid expansion protocol (REP) for 14 days. Following expansion, 1×10⁵ cells were assessed for transduction efficiency (FIG. 23) either via surface detection of the marker gene tCD34 or CoStAR molecule using an anti-CD34-APC (black bars) or using a primary rhCEACAM5-Fc antibody with a secondary anti-IgG-Fc-PE (grey bars) antibody, respectively.

### Example 9

Anti-CEA CoStAR Activity

Cytokine production was assessed in CoStAR transduced HD T cells cocultured with Lovo target cell lines. CoStARs comprising different anti-CEA binding domains (Table 12) were tested. Nontransduced (MOCK) and anti-CEA CoStAR transduced HD T cells were cocultured with engineered target cell lines at an effector to target (E:T) ratio of 8:1 (1×10⁵: 1.25×10⁴) for 24 hours and MSD immunoassay was performed to evaluate the concentration of cytokines secreted. Cytokine concentrations were determined for IL-2 (FIG. 24A) IFNγ (FIG. 24B) and TNFα (FIG. 24C) following cocultures with Lovo or Lovo-OKT3 cell lines. Non-treated T cells were used as a control. The results represent 1 biological replicate with 3 technical replicates.

Cytokine production was also assessed in CoStAR transduced HD T cells cocultured with K562 target cell lines. As above, cells were cocultured with engineered target cell lines at an effector to target (E:T) ratio of 8:1 (1×10⁵: 1.25×10⁴) for 24 hours and MSD immunoassay was performed to evaluate the concentration of cytokines secreted and cytokine concentrations were determined for IL-2 (FIG. 25A) IFNγ (FIG. 25B) and TNFα (FIG. 25C) following cocultures with K562.CEACAM5 or K562.CEACAM5.OKT3 cell lines. Non-treated T cells were used as a control.

Spacer-transmembrane variants were also examined. In one experiment, anti-CEA CoStARs comprising an hMFE23 CEA-binding domain and different spacer/transmembrane domains (Table 13) were compared.

TABLE 13

| Clone | CTP220 | CTP232 | CTP244 |
|---|---|---|---|
| SEQ ID NO: | 78 | 84 | 162 |
| Signal Peptide | OSM1 | | |
| scFv | hMFE23 | | |
| linker | AAAGSGGSG | | |
| Spacer | CD28 EC | CD8 EC | CD28 (trunc IIH) |
| TM | CD28 | CD8 | CD28 |

| | |
|---|---|
| Intracellular | CD28.CD40 |

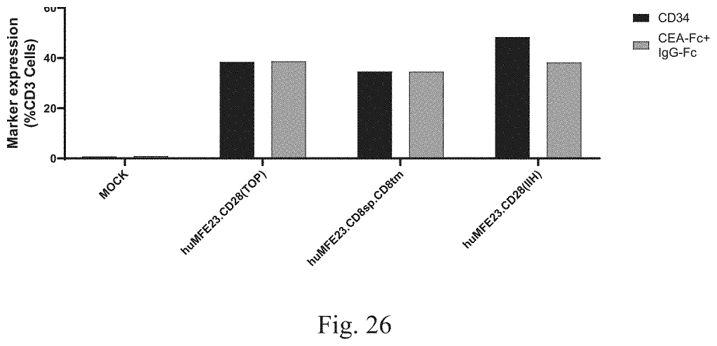

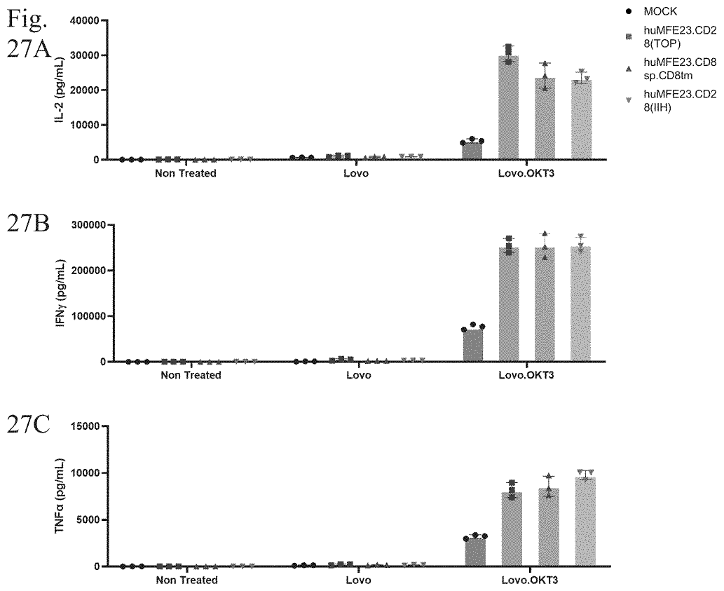

T cells from healthy donor PBMCs were lentivirus transduced at a multiplicity of infection (MOI) 5, with the hMF23 scFV constructs against the carcinoembryonic antigen 5 (CEA) that possessed CD28.CD40 domains. Cells were sorted using CD34 microbeads and underwent a rapid expansion protocol (REP) for 14 days. Following expansion, 1×10⁵ cells were assessed for transduction efficiency (FIG. 26) via surface detection of the marker gene tCD34 using an anti-CD34-APC (black bars) or detection of the CoStAR molecule or using a primary rhCEACAM5-Fc antibody with a secondary anti-IgG-Fc-PE (grey bars) antibody. All variants were efficiently expressed.

Cytokine production was assessed in CoStAR transduced HD T cells cocultured with Lovo target cell lines. As above, cells were cocultured with engineered target cell lines at an effector to target (E:T) ratio of 8:1 (1×10⁵: 1.25×10⁴) for 24 hours and MSD immunoassay was performed to evaluate the concentration of cytokines secreted and cytokine concentrations were determined for IL-2 (FIG. 27A) IFNγ (FIG. 27B) and TNFα (FIG. 27C) following cocultures with Lovo or Lovo-OKT3 cell lines. Non-treated T cells were used as a control.

### Example 10

CoStARs were constructed to test intracellular signaling domains. FIG. 28 and Table 14 depict anti-CEA CoStARs comprising an hMFE23 CEA-binding domain with intracellular signaling domains comprising CD40, CD134, CD137, CD2, ICOS, DAP10, and NTRK1 signaling elements.

TABLE 14

| Clone | CTP313 | CTP314 | CTP315 | CTP316 | CTP317 |
|---|---|---|---|---|---|
| SEQ ID NO: | 344 | 345 | 346 | 347 | 348 |

| Signal peptide | OSM1 | |
|---|---|---|
| scFv | hMFE23 | |
| Linker | AAAGSGGSG | |
| Spacer | CD28 EC | ICOS |
| TM | CD28 | ICOS |

| | | | | | |
|---|---|---|---|---|---|
| Intracellular | NTRK1 | NTRK1 | CD28 | CD28 | ICOS |
| | | CD40 | NTRK1 | NTRK1 | CD40 |
| | | | | CD40 | |

| Clone | CTP318 | CTP319 | CTP320 | CTP321 | CTP322 | CTP323 |
|---|---|---|---|---|---|---|
| SEQ ID NO: | 349 | 350 | 351 | 352 | 353 | 354 |
| Signal peptide | OSM1 | | | | | |
| scFv | hMFE23 | | | | | |
| Linker | AAAGSGGSG | | | | | |
| Spacer | CD28 EC | | CD2 EC | CD28 EC | | |
| TM | CD28 | | CD2 | CD28 | | |
| Intracellular | CD28 | CD28 | CD2 | CD28 | CD28 | CD28 |
| | ICOS | ICOS | CD40 | CD2 | CD40 | CD137 |
| | | CD40 | | | CD2 | |

| Clone | CTP324 | CTP325 | CTP326 | CTP327 | CTP328 |
|---|---|---|---|---|---|
| SEQ ID NO: | 355 | 356 | 357 | 358 | 359 |
| Signal peptide | OSM1 | | | | |
| scFv | hMFE23 | | | | |
| Linker | AAAGSGGSG | | | | |
| Spacer | CD28 EC | | | | |
| TM | CD28 | | | | |
| Intracellular | CD28 | CD28 | CD28 | CD28 | CD28 |
| | CD40 | DAP10 | CD40 | CD134 | CD40 |
| | CD137 | | DAP10 | | CD134 |

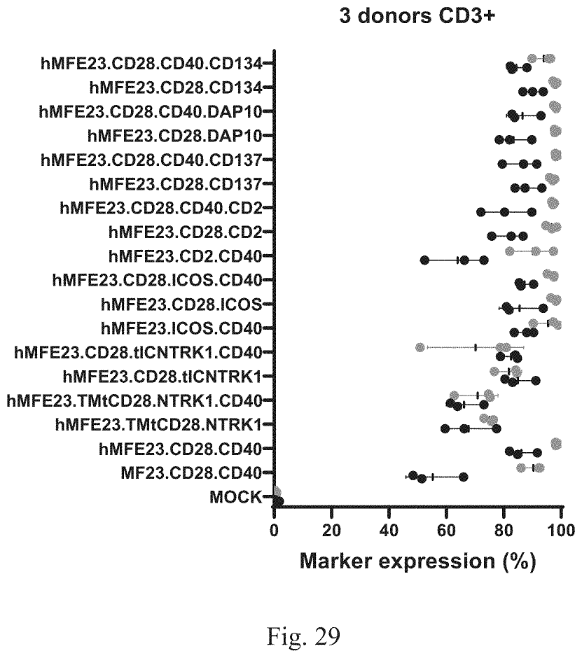

T cells from healthy donor PBMCs were lentivirus transduced at a multiplicity of infection (MOI) 5 with the hMF23 scFV constructs against the carcinoembryonic antigen 5 (CEA). Nontransduced (MOCK) cells were used as controls. Cells were sorted using CD34 microbeads and underwent a rapid expansion protocol (REP) for 14 days. Following expansion, 1×10⁵ cells were assessed for transduction efficiency (FIG. 29) either via surface detection of the marker gene tCD34 or CoStAR molecule using an anti-CD34-APC (black) or using a primary rhCEACAM5-Fc antibody with a secondary anti-IgG-Fc-PE (red) antibody, respectively. The results represent 3 biological replicates.

CoStAR transduced cells were phenotypically characterized. Following outgrowth and REP, 1×10⁵ cells were assessed for the differentiation subtype using flow cytometry.

TABLE 15
T Cell Differentiation Subtype Definition

| | | | |
|---|---|---|---|
| TN | CD45RO− | CCR7+ | CD95− |
| Tscm | CD45RO− | CCR7+ | CD95+ |
| Tcm | CD45RO+ | CCR7+ | CD95+ |
| Tem | CD45RO+ | CCR7− | CD95+ |
| Tte | CD45RO− | CCR7− | CD95+ |

Tcm, central memory T cell;
Tem, effector memory T cell;
Tn, naïve T cell;
Tscm; stem cell memory T cell;
Tte, terminal effector T cell

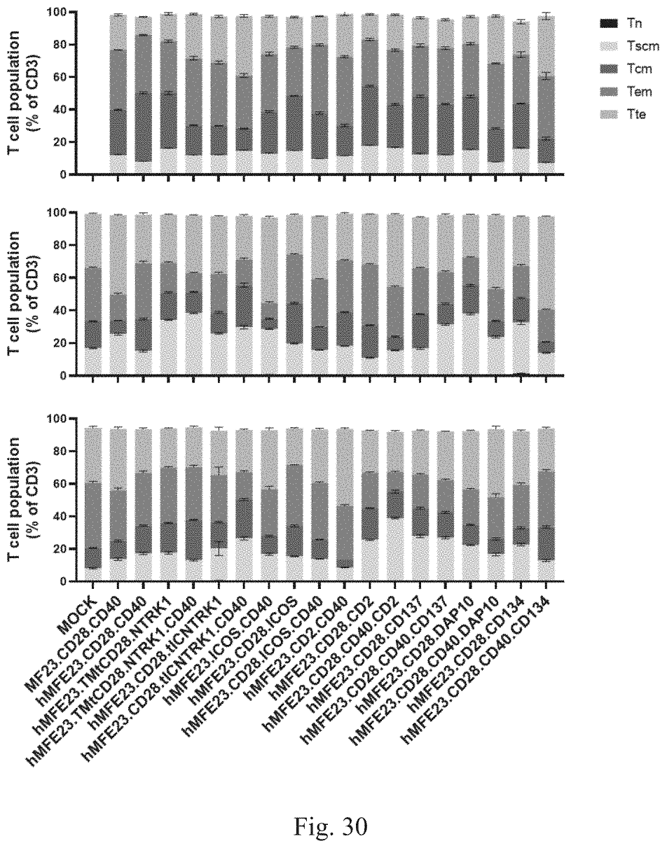

T cells from HD PBMCs of three donors were lentivirus transduced with the hMF23 scFV constructs of FIG. 28. Cells were sorted using CD34 microbeads and underwent a rapid expansion protocol (REP) for 14 days. Following outgrowth and REP, 1×10⁵ cells were assessed for the differentiation subtype compared to non-transduced cells using flow cytometry. T cell phenotypes are depicted in FIG. 30 as a proportion of CD3 cells.

### Example 11

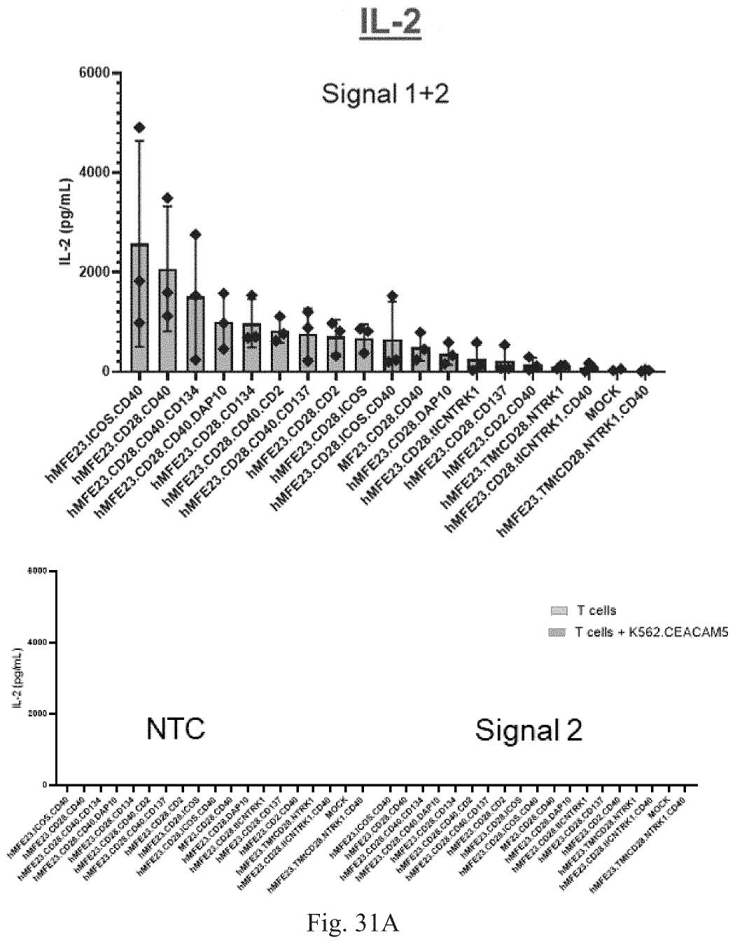

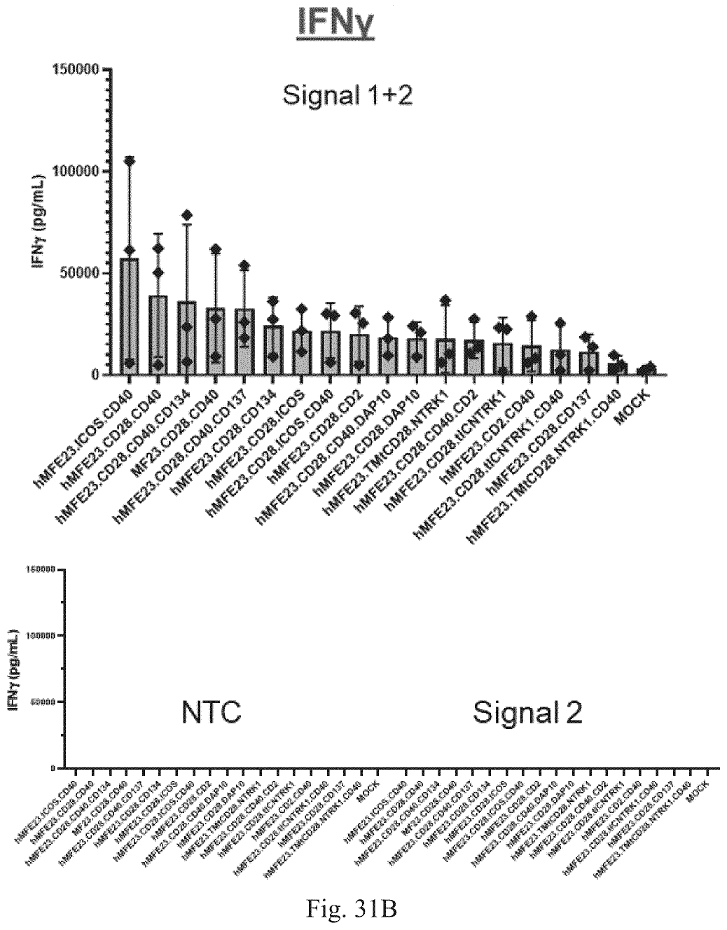

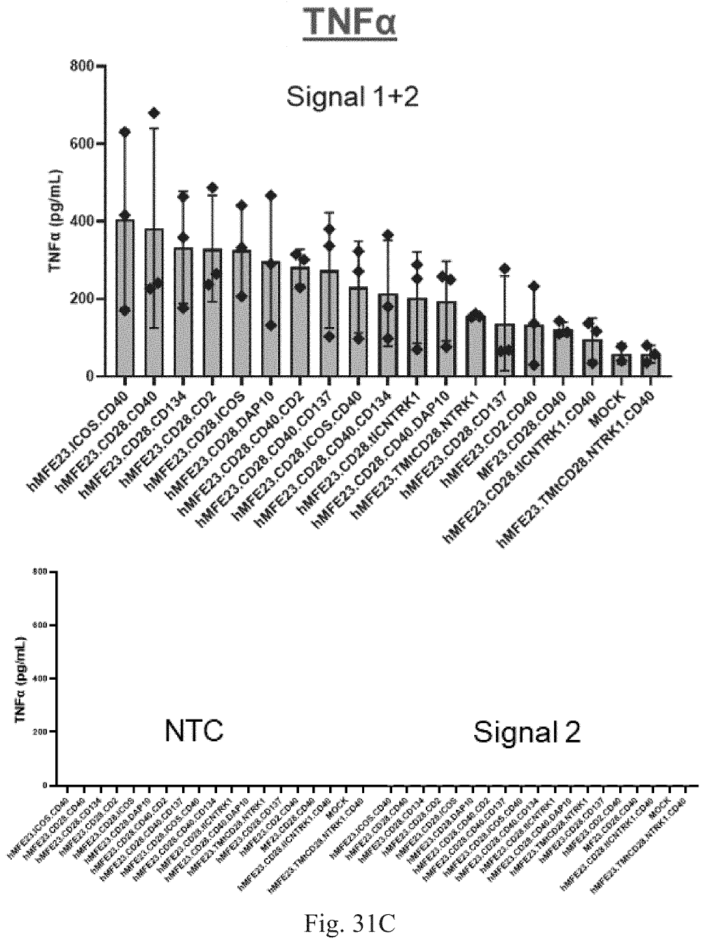

Cytokine secretion anti-CEA hFME23 CoStAR transduced T cells was assessed by coculture with K562 target cells. Nontransduced (MOCK) and anti-CEA CoStAR transduced HD T cells were cocultured with engineered target cell lines at an effector to target (E:T) ratio of 8:1 (1×10⁵: 1.25×10⁴) for 24 hours and MSD immunoassay was performed to evaluate the concentration of cytokines secreted. Cytokine concentrations for IL-2 (FIG. 31A) IFNγ (FIG. 31B) and TNFα (FIG. 31C), following cocultures with K562.CEACAM5 (signal 2) or K562.CEACAM5.OKT3 (signal 1+2) cell lines are shown. Non-treated T cells were used as a control.

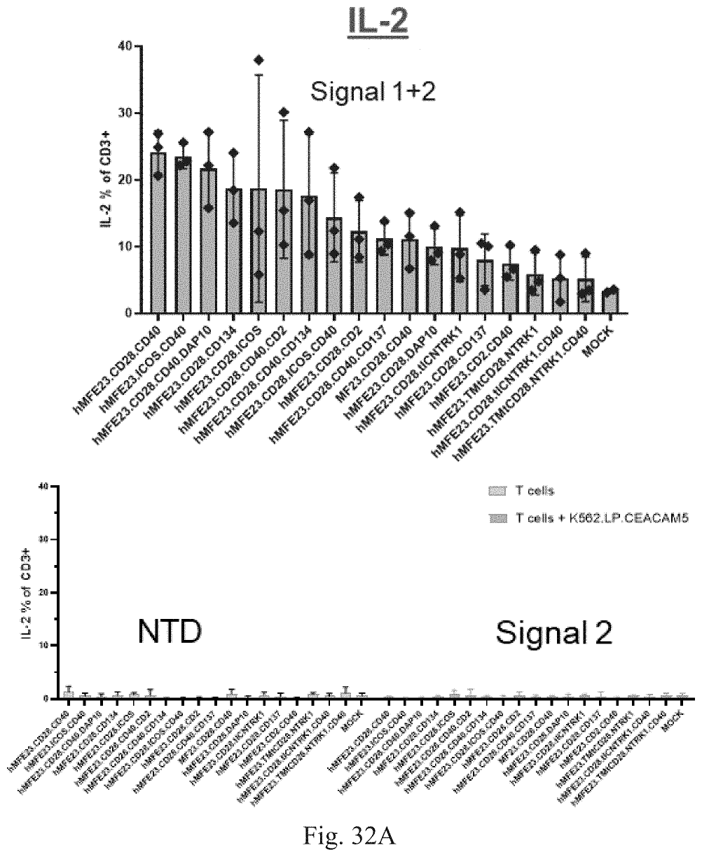

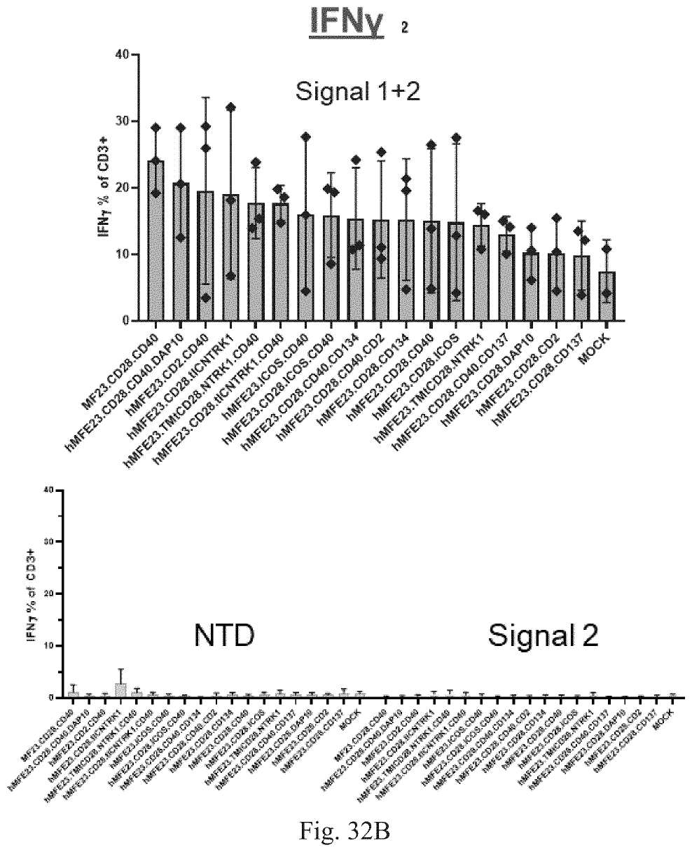

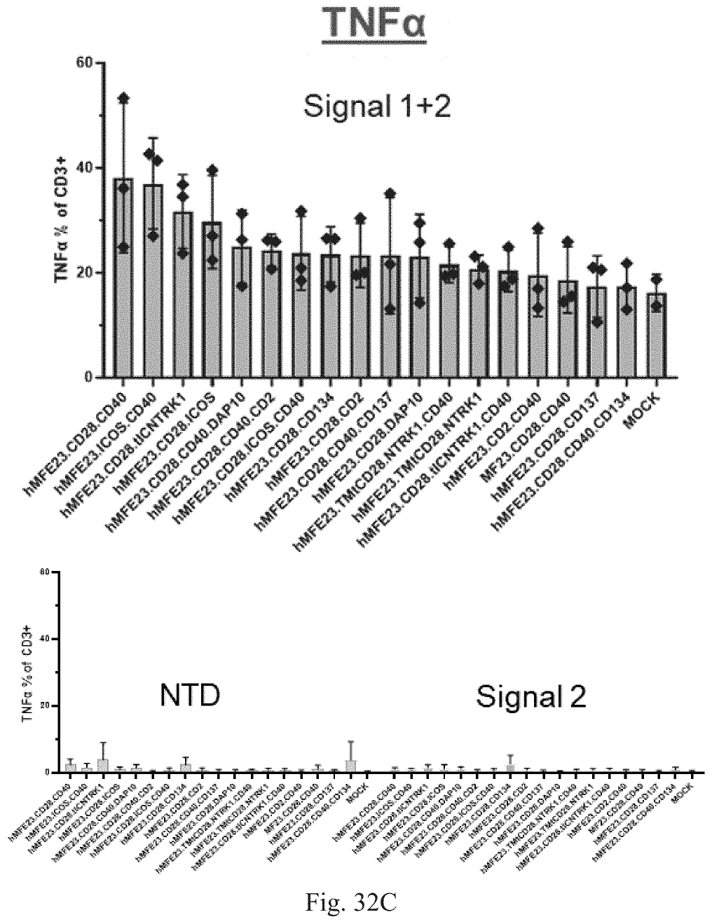

Cytokine expression was also assessed. Nontransduced (MOCK) and anti-CEA CoStAR transduced HD T cells were cocultured with engineered target cell lines at an effector to target (E:T) ratio of 1:1 (1×10⁵: 1×10⁵) for 16 hours in the presence of Brefeldin A and cytokine producing cells were measured using intracellular flow cytometry. Frequency of IL-2 (FIG. 32A) IFNγ (FIG. 32B) and TNFα (FIG. 32C) expressing cells following cocultures with K562.CEACAM5 (signal 2) or K562.CEACAM5.OKT3 (signal 1+2) cell lines are shown. Non-treated T cells were used as a control.

### Example 12

Proliferation of Anti-CEA CoStAR Transduced Cells.

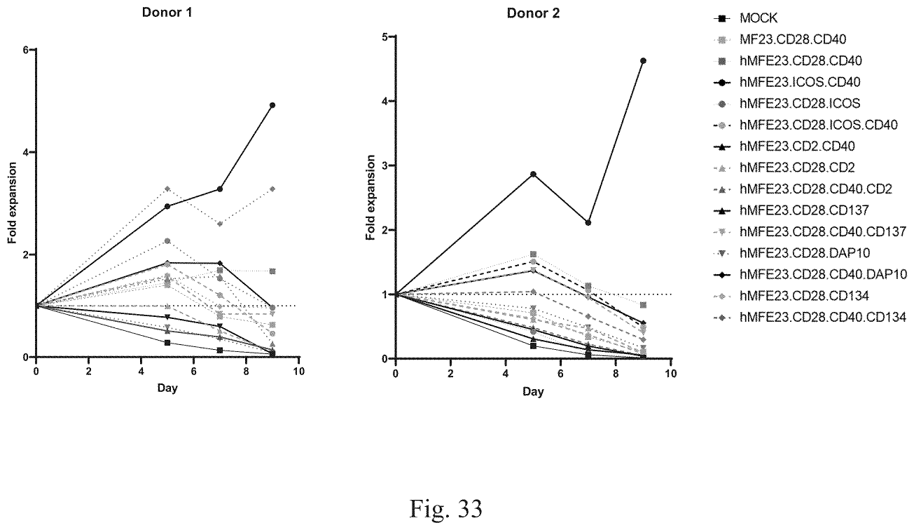

HD T cells were transduced with hMFE23 anti-CEA CoStARs and cocultured with engineered target cells. Nontransduced (MOCK) and anti-CEA CoStAR transduced HD T cells were cocultured with K562.CEACAM5.OKT3 engineered target cell lines at an effector to target (E:T) ratio of 8:1 (1×10⁵: 1.25×10⁴) on Day 0. Nontransduced (MOCK) cells were used as controls. On Day 7, a maximum 50000 cells were re-stimulated with K562.CEACAM5.OKT3 engineered target cell lines at an E:T of 8:1. On Day 5, Day 7 and Day 9 post stimulation a portion of the cocultures were collected for counting by flow cytometry. For all counts, DRAQ7 was used for live cell discrimination and CD2 to enumerate the T cells (FIG. 33). The figures represent fold expansion of input cells. N=2

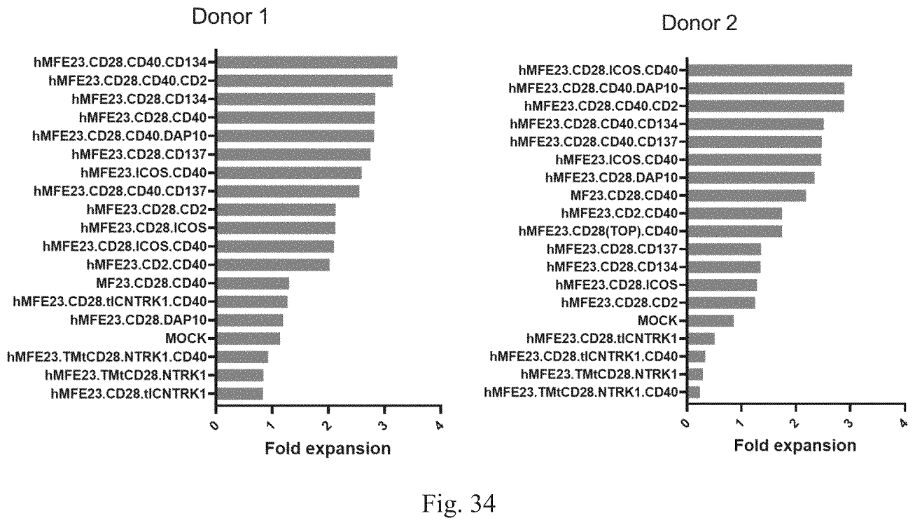

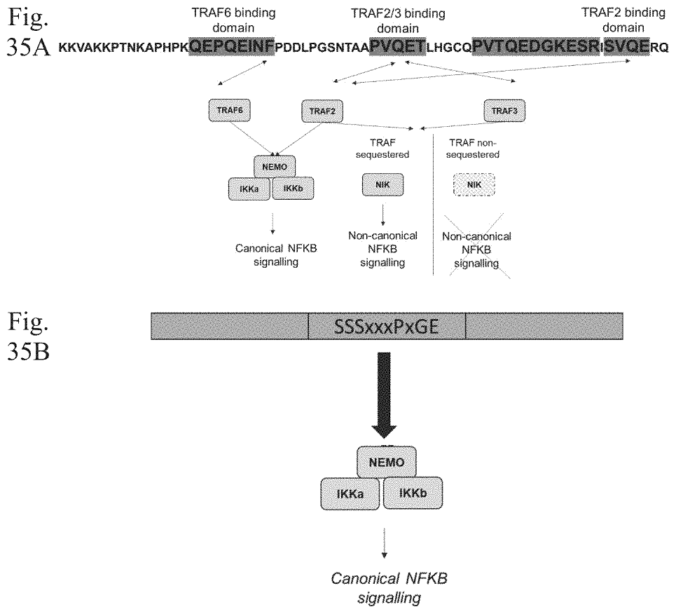

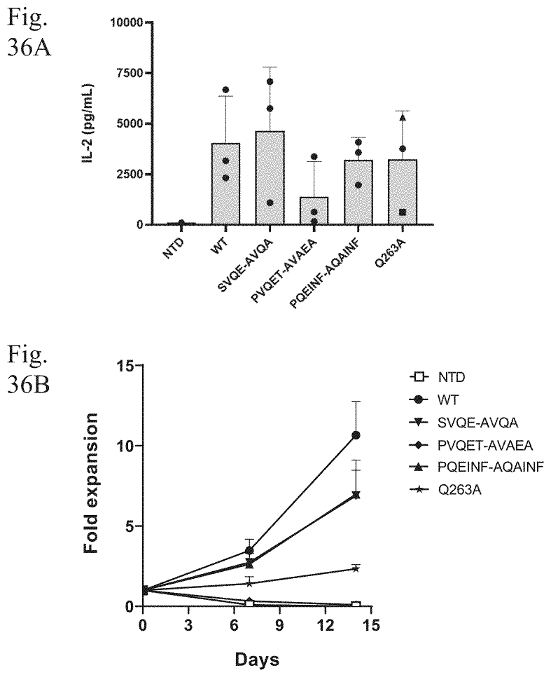

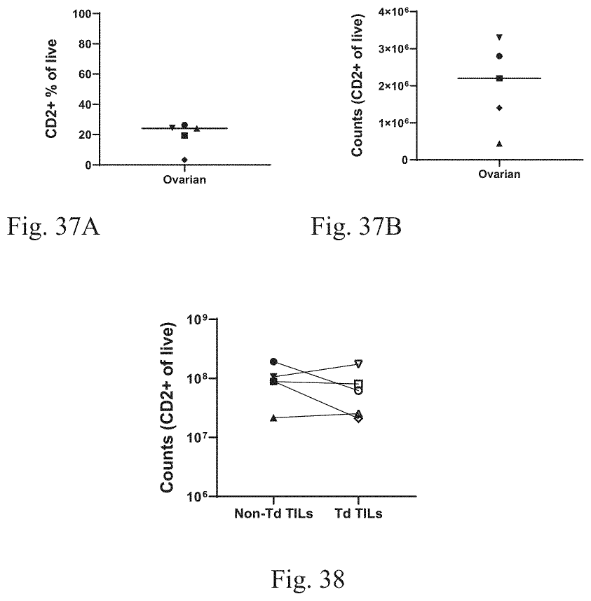

HD T cells transduced with hMFE23 anti-CEA CoStARs and cocultured with K562.CEACAM5.OKT3 engineered target cell lines as above were sampled for counting on Day 6 to evaluate fold expansion (FIG. 34). Cells were counted by flow cytometry using DRAQ7 for live cell discrimination and CD2 to enumerate the T cells.

### Example 13

Signal Transduction and Intracellular Domain Binding Sites and Motifs.

The effect of mutations in TRAF2/TRAF3 and TRAF6 binding sites (FIG. 35) of CD40 on cytokine secretion and long term survival and proliferation of CD28.CD40 CoStAR transduced T cells was examined. Cells of three donors were activated with Dynabeads and transduced with WT CD28.CD40 (CTP194), CD28.CD40 containing TRAF2 binding site mutation SVQE>AVQA (CTP195), TRAF2/TRAF3 binding site mutation PVQET>AVAEA (CTP196), TRAF6 binding site mutation PQEINF>AQAINF (CTP197), Q263A (CTP199), or mock transduced. Cells were enriched for CD34 marker expression, expanded following the rapid expansion protocol (REP) and frozen for subsequent experiments. After thaw, cells were rested for 3-4 days in complete RPMI supplemented with IL-2 and their transduction rate was determined looking at the CD34 marker gene expression. The viability and absolute count were assessed after overnight IL-2 starvation using DRAQ-7 (1:200) by flow cytometry (Novocyte) and data were analysed using the NovoExpress 1.5.0 software. Transduced T cells were cocultured in absence of IL-2 with LoVo (CCL-229™ from ATCC) or LoVo.OKT3.GFP tumor cells at 8:1 effector to target ratio. After 24 hours, supernatants were collected and frozen. LoVo and LoVo.OKT3.GFP naturally express CEA and PD-L1 on their surface, conferring signal 2 through the CoStAR alone (LoVo) or associated with signal 1 (LoVo.OKT3.GFP) to the transduced T cells. Cocultures were performed in triplicates and corresponding negative (T cells alone, tumor cells alone) and positive (PMA+ ionomycin) controls were included in the experiment. After thaw, secreted IL-2 was detected by ELISA and the absorbance was measured using the FLUOstar Omega microplate reader and subsequently analysed with the Omega MARS 3.42 R5 software (FIG. 36A). Each dot represents the mean of triplicates for one donor. Note that negative controls (T cells alone, tumor cells alone) were all below the detection range.

After 6-8 days, the viability and absolute count were assessed, and live T cells were rechallenged for an additional week with fresh LoVo.OKT3.GFP tumor cells as described above. At the end of the long-term coculture, the viability and absolute count were measured, and the fold expansion was calculated (FIG. 36B). Data shown as mean+/−SEM of n≤3 donors analysed in triplicates.

Mutation of the TRAF2 binding site (SVQE>AVQA; CTP195) resulted little reduction in IL-2 secretion and moderate reduction of expansion (FIG. 80). Mutation of the TRAF2/TRAF3 binding site (PVQET>AVAEA; CTP196) resulted in substantial reduction in IL-2 secretion and expansion. Mutation of the TRAF6 binding site (PQEINF>AQAINF; CTP197) resulted in moderate reduction in IL-2 secretion and moderate reduction of expansion.

### Example 14

Non-transduced (Non-Td) and anti-FOLR1 CoStAR transduced (Td) TILs were generated using a 24-day protocol. Briefly, aliquots of OC digest were thawed and transduced with anti-FOLR1 CoStAR lentivirus at a multiplicity of infection (MOI) of 5 at 48 h and 72 h. Cells were then expanded for 8 days (outgrowth), and then subjected to a rapid expansion protocol (REP) with allogeneic irradiated peripheral blood mononuclear cells (PBMCs) for 12 days.

After production, TIL CD4/CD8 ratio and anti-FOLR1 CoStAR transduction efficiency was measured. TILs were phenotypically characterized for their differentiation status, expression of co-inhibitory and co-stimulatory markers, T cell subsets and cytokine producing potential using flow cytometric panels.

Complete TIL T cell media (TCM) for outgrowth consists of 450 mL of GIBCO custom P158718 media with 50 mL of heat inactivated Fetal Bovine Serum, gentamycin (10 μg/mL)/amphotericin (0.25 μg/mL) and vancomycin (50 μg/mL). Complete rapid expansion protocol (REP) media consists of 460 mL of GIBCO custom P158718 with 40 mL human AB serum, gentamycin (10 μg/mL)/amphotericin (0.25 μg/mL) and vancomycin (50 μg/mL).

Generation of Anti-FOLR1 CoStAR OC TILs

Five OC samples were used to generate anti-FOLR1 CoStAR modified TILs. The outgrowth period of TILs was 12 days. On day 1 (D1), samples from each donor were thawed in complete TIL TCM, the cells were washed once by centrifuging at 400×g for 5 minutes, resuspended in fresh TIL TCM and counted. All cell counts were performed using a DRAQ7 dye and anti-CD2 antibody stains using a Novocyte 3005 Flow Cytometer System. TILs were then centrifuged at 400×g for 5 minutes, resuspended at a concentration of 1×10⁶ cells/mL, placed into an appropriate vessel with 3000 IU/mL IL-2 and rested for two days in a 5% CO2 incubator set to 37° C.

Following the rest period on day 3, cells were collected, washed, centrifuged at 400×g for 5 minutes, and resuspended in fresh complete TCM. The number of viable cells in each sample was determined using a Novocyte 3005 as described above, cells were centrifuged at 400×g for 5 minutes and resuspended at a concentration of 1×10⁶ cells/mL. Each sample was split into two equal parts, one for production of Non-Td and other for transduced (Td) TIL, modified to express anti-FOLR1 CoStAR. Transduction with anti-FOLR1 CoStAR lentivirus was performed at an MOI of 5 based on the total number of live cells. IL-2 was added at a concentration of 3000 IU/mL and the cells were placed in a 5% CO2 incubator set to 37° C.

On day 4, the cells were collected, washed once with complete TIL TCM, and resuspended in the same volume of fresh complete TIL TCM as on day 3 for the second day of transduction. Transduction was performed using the anti-FOLR1 CoStAR lentivirus at an MOI of 5 and IL-2 at 3000 IU/mL was added to the cells prior to placing them in a 5% CO2 incubator set to 37° C. for 8 days. IL-2 (3000 IU/mL) was added to the cells every 2-3 days until D13.

On day 13 cells were collected, washed, resuspended in complete TIL TCM and counted using a Novocyte 3005. After determining the TIL numbers on day 13, the cells were seeded in appropriate scale G-REX plates for REP using 10 healthy donors worth of irradiated allogeneic PBMCs as feeders at a 200:1 ratio of feeders:TIL. The media used for the REP was the complete REP TIL TCM with anti-CD3 (OKT3) antibody was added at a concentration of 30 ng/mL for activation. IL-2 was added at a concentration of 3000 IU/mL and the cells were placed in a 5% CO2 incubator set to 37° C. The REP period was 12 days during which IL-2 (3000 IU/mL) was supplemented every 2-3 days.

On day 19 (i.e., day 6 of REP), 5 mL of medium from the 24 well G-REX plates or 25 mL of medium from the 6 well G-REX plates was removed without disturbing the cells and replaced with fresh complete REP TIL TCM and IL-2 (3000 IU/mL). On D25, at the end of the REP, TILs were harvested by centrifugation at 400×g for 5 minutes and resuspended in fresh media for counting using a Novocyte 3005. TILs were resuspended at a concentration of 1×10⁶ cells/mL. TILs were then assessed for transduction efficiency by staining 1×10⁵ cells of each sample with antibodies against CD3, CD4, CD8, CoStAR and a viability stain. DNA was extracted from 1×10⁶ cells from each sample using the DNeasy Blood & Tissue Kit following the manufacturer's instructions. Isolated DNA was used to analyze the vector copy number (VCN) using Droplet Digital PCR (ddPCR) and primers specific to the anti-FOLR1 CoStAR and the reference gene Poly(rC) binding protein 2 (PCBP2). For subsequent experiments 2-5×10⁷ cells were rested in fresh complete REP TIL TCM for 3 days with IL-2 (3000 IU/mL). Remaining TIL were resuspended in cryoprotectant and aliquoted to cryovials, cooled to −80° C. overnight, and then transferred to −150° C. for short term storage. Cryopreserved TIL were thawed in a 37° C. water bath, washed once with PBS by centrifugation at 400×g for 5 minutes, then underwent an identical rest period as described above prior to experimentation.

Phenotypic characterization of anti-FOLR1 CoStAR OC TILs from four donors.

Non-Td and Td cells from four donors were rested for 3 days in REP TCM media with IL-2 (3000 IU/mL). Subsequently, the cells were harvested, washed once with media by centrifugation at 400×g for 5 minutes, resuspended in fresh complete REP TIL TCM, counted using Novocyte 3005, and resuspended at a concentration of 1×10⁶ cells/mL. Cytometric evaluation of TILs was performed on 1×10⁵ cells per well, in triplicates, using four flow cytometry panels. Assessment of differentiation status was performed using a panel with antibodies against CD3, CD4, CD8, CD27, CD95, CCR7, CD45RA, CD45RO, CoStAR, and a viability stain. Coinhibitory and costimulatory marker expression was assessed using antibodies against CD4, CD8, CD137, programmed cell death protein 1 (PD-1), cytotoxic T-lymphocyte-associated protein 4 (CTLA-4), lymphocyte-activation gene 3 (LAG-3), T cell immunoglobulin and mucin domain protein 3 (TIM-3), signaling lymphocyte activation molecule (SLAM), CoStAR, and a viability stain. Cell subpopulations, including Tregs, were assessed using antibodies against CD3, CD4, CD25, forkhead box Protein 3 (FOXP3), T cell receptor alpha beta (TCRαβ), T cell receptor gamma delta (TCRγ6), CD56, CD127, CoStAR, and a viability stain. Cytokine production upon mitogenic stimulation was assessed using antibodies against CD3, CD4, CD8, IL-22, TNFα, IL-17A, IFNγ, CoStAR, and a viability stain. For this panel, TILs were activated by addition of PMA (50 ng/mL)/ionomycin (1 μg/mL), and Brefeldin A (1000×) and placed in a 5% CO2 incubator set to 37° C. for four hours. Following activation, TILs were collected, centrifuged at 400×g for 5 minutes, counted, and 1×10⁵ cells were seeded in triplicate for cytometric analysis. For all flow cytometry panels, fixation and permeabilization was performed using BD Cytofix/Cytoperm per manufacturer's instructions. Following staining, cells were washed and resuspended in PEF (500 mL DPBS, 2 mL EDTA and 2.5 mL of heat inactivated FBS) for analysis using a Novocyte 3005 Flow Cytometer System.

### Analysis of the Non-Td and Td Cells

For the four panels of phenotypic characterization, recombinant human FOLR1 with Fc tag (rhFOLR1-FC) was used for the detection of anti-FOLR1 CoStAR cells, and the populations of interest were reported from CoStAR− subset for the Non-Td TILs, and both CoStAR- and CoStAR+ fractions from the Td TILs (anti-FOLR1 CoStAR− Td TILs and anti-FOLR1 CoStAR+Td TILs). Further subset characterization was performed on these populations.

Differentiation Status

The gating strategy employed for characterizing T cell differentiation status was as follows: a lymphocyte gate followed by doublet and dead cell exclusion, CD3+, CD4+ and CD8+ gates. From all three populations (CD3+, CD4+, and CD8+), further analysis was performed on the T cell fractions of interest. To characterize the different T cell memory subsets, CD45RA+CD45RO− and CD45RA-CD45RO+ cells were gated from CD3+ cells. CD45RO+ CCR7+ and CD45RO+ CCR7− populations were then gated from CD45RA−CD45RO+ cells. Using these gates, the central memory T cells (Tcm; CD45RO+ CCR7+CD95+CD27+) cells and effector memory T cells (Tem; CD45RO+ CCR7−CD95+CD27+/−) cells were further gated. Additionally, CD45RA+ CCR7+ and CD45RA+ CCR7− populations were gated from CD45RA+CD45RO− cells. Using these gates, stem cell memory T cells (Tscm; CD45RA+ CCR7+CD95+CD27+) and naïve T cells (Tn; CD45RA+ CCR7+CD95−CD27+) were gated from CD45RA+ CCR7+ cells, whilst terminal effector T cells (Tte; (CD45RA+ CCR7−CD27−CD95+) cells were gated from CD45RA+ CCR7− cells.

Coinhibitory and Costimulatory Markers

The gating strategy employed for characterizing coinhibitory and costimulatory molecules included doublet and dead cell exclusion. Gating of CD4+ and CD8+ and CoStAR+/− cells was performed and populations were further analyzed for CD137, PD-1, CTLA-4, LAG-3, TIM-3 and SLAM expression.

T Cell Subtype

The gating strategy employed for characterizing T cell subtypes included doublet and dead cell exclusion and a CD3+ gate. Using these populations, the expression of TCRαβ, TCRγδ, and CD56 was assessed. Subsequently, the CD3+CD4+ cells were gated for expression of TCRαβ and CD56, and further analysis of the CD3+CD4+TCRαβ+ population for CD25 and CD127 expression was performed. Using the CD25+CD127−gate the FOXP3+ cells were gated to determine the population of Tregs. Therefore, Tregs are defined as CD3+TCRab+CD4+CD25+CD127-FOXP3+ and CoStAR+/− depending on the population assessed.

Cytokine Production Upon Mitogenic Stimulation

The gating strategy employed for characterizing intracellular cytokine production included doublet and dead cell exclusion, CD3+, CD4+, and CD8+ gates. CoStAR expression analysis on the different populations was performed followed by further analysis of the frequency of cells expressing IL-22, IL-17A, TNFα, and IFNγ.

Clinical Characteristics

Clinical characteristics of TILs from five ovarian cancer patients are shown in Table 16. Patients were all treatment naïve, and the specific histology revealed three serous cystadenocarcinomas, one endometrioid, and one clear cell adenocarcinoma. Sample weights ranged between 0.55-2.62 grams with a mean weight of 2.39±1.31 grams. After processing, samples were cryopreserved at 6×10⁶-2.5×10⁷ cells in 1 mL per vial. On day 1, samples were thawed and counted using flow cytometry where the average percentage of CD2+ TILs was 19.5±9.43. The total TILs harvested in the thawed samples ranged between 4.4×10⁵-3.3×10⁶ cells.

TABLE 16
Ovarian tumor sample information

| | Tumor type/ | Tumor | | Clinical | | | Treatment |
|---|---|---|---|---|---|---|---|
| ID | Histology | weight | Markers | stage | TNM | Grade | status |
| 1 | Ovarian Cancer/ | 2.62 | CA125- | IB | T1b- | G2-Moderately | Treatment- |
| | Serous | | 937 | | N0-M0 | differentiated | naïve |
| | cystadenocarcinoma- | | | | | | |
| | M-84413 | | | | | | |
| 2 | Ovarian Cancer/ | 0.55 | CA125- | IV | T3-Nx- | G3-Poorly | Treatment- |
| | Serous | | 478, 83 | | M1 | differentiated | naïve |
| | cystadenocarcinoma- | | | | | | |
| | M-84413 | | | | | | |
| 3 | Ovarian | 4.2 | CA125- | IIB | T2b- | G3-Poorly | Treatment- |
| | Cancer/Serous | | 50, 53 | | Nx-M0 | differentiated | naïve |
| | cystadenocarcinoma- | | | | | | |
| | M-84413 | | | | | | |
| 4 | Ovarian Cancer/ | 2.1 | CA125- | IC | T1c- | G2-Moderately | Treatment- |
| | Endometrioid | | 1096 | | Nx-M0 | differentiated | naïve |
| | carcinoma-M- | | HE4- | | | | |
| | 83803 | | 1487 | | | | |
| 5 | Ovarian Cancer/ | 2.5 | CA125- | IIIC | T3c- | G3-Poorly | Treatment- |
| | Clear cell | | 187 | | Nx-M0 | differentiated | naïve |
| | adenocarcinoma-M- | | | | | | |
| | 83103 | | | | | | |

Abbreviations: ID, identification; CA125, cancer antigen 125; TNM, tumor, nodes, metastases.

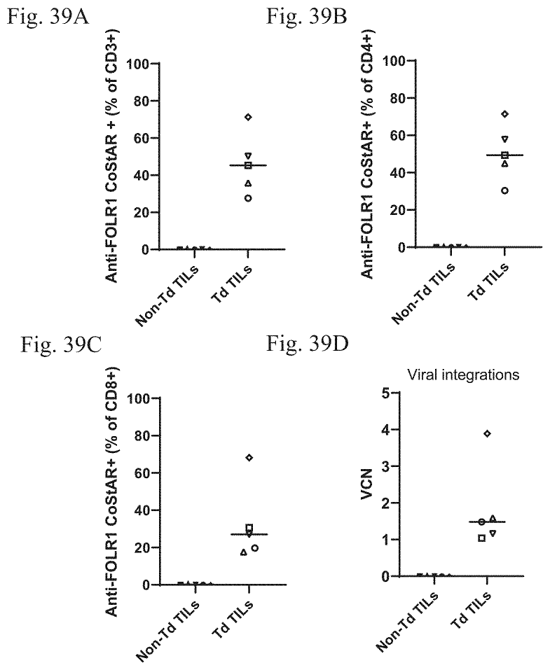

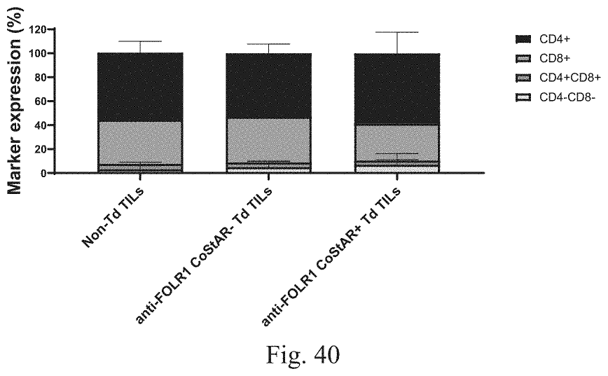

After TIL production, cell numbers of Non-Td and Td TILs were compared to assess any effect of anti-FOLR1 CoStAR modification on TIL growth. Results showed no significant impact on the cell numbers of TILs (FIG. 38). Proportions of transduced CD3+, CD4+ and CD8+ cells were assessed after TIL production (FIG. 39A-C). CD4 cells showed higher transduction percentage (50.8±15.2%) than the CD8 cells (32.7±20.6%), and this was statistically significant (p=0.384). Additionally, VCN was assessed using ddPCR to detect the CoStAR transgene relative to the PCBP2 reference gene and <4 integrations per cell were measured (FIG. 39). Comparison of the CD4 and CD8 population composition in the product indicated most cells were CD4+ with levels ranging between 53.0-58.7% and fewer CD8+ cells (30.8-38.2%). CD4+CD8+ and CD4−CD8− cells were also detected at low levels ranging between 3.47-4.43% and 3.19-6.97%, respectively. There were no significant differences between the Non-Td, anti-FOLR1 CoStAR-Td, and anti-FOLR1 CoStAR+Td TILs with regards to the CD4/CD8 cell composition (FIG. 40).

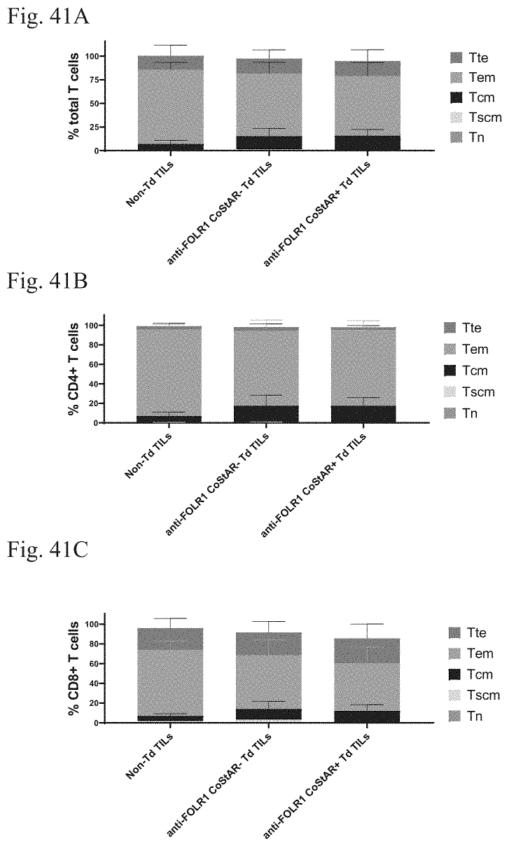

The TILs were further characterized to determine whether anti-FOLR1 CoStAR modification impacted TIL function. The composition of Tn, Tscm, Tcm, Tem and Tte were assessed from CD3+, CD4+ and CD8+ T cell compartments of Non-Td, anti-FOLR1 CoStAR− Td and anti-FOLR1 CoStAR+Td TILs (FIG. 41A-C). Results show that majority of TILs were Tem across the CD3+(63.2-78.6%), CD4+(76.9-88.8%) and CD8+(48.3-66.9%) populations. Tcm cell frequencies were between 6.28-15.7%, 6.72-17.5% and 5.18-11.7% for CD3+, CD4+ and CD8+ TILs, respectively. Tte cells made up 14.8-15.9% of bulk CD3+ TILs. These were mainly in the CD8+ subpopulation with frequencies of 22.1-25.1% versus 2.68-3.79% in the CD4+ subpopulation. Few Tscm cells were detected for CD3+, CD4+ and CD8+ TILs with frequencies of 0.15-1.38%, 0.05-0.50% and 0.53-3.24%, respectively. The lowest frequency memory subtype were the Tn cells, and these ranged between 0.002-0.019% across all three populations. There were no significant differences between the Non-Td, anti-FOLR1 CoStAR− Td and anti-FOLR1 CoStAR+Td TILs with regards to Tn, Tem, Tcm or Tte frequencies. Despite the low Tscm TILs, significantly lower Tscm TILs were detected in the anti-FOLR1+Td TILs in CD3+(0.15±0.05% vs 1.38±0.69%) and CD4+(0.05±0.02% vs 0.51±0.27%) populations compared to the anti-FOLR1− Td TILs, but not the Non-Td TILs. Overall, the data shows that anti-FOLR1 CoStAR modification did not significantly affect differentiation status of TILs.

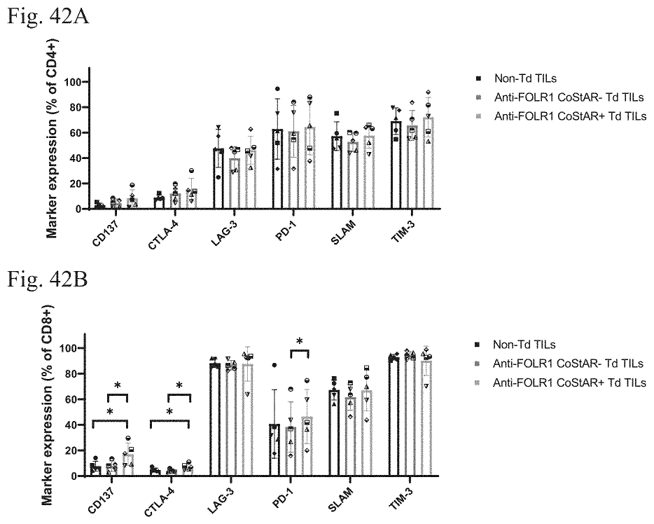

CD8+ and CD4+ TILs were assessed for the expression of CD137, PD-1, CTLA-4, LAG-3, TIM-3 and SLAM (FIG. 42). In CD4+ TILs, expression for all three populations of interest were in the range of 39.8-47.7%, 61.0-64.4%, 52.7-57.6, and 65.6-72.1% for LAG-3, PD-1, SLAM, and TIM-3, respectively. In CD8+ TILs, expression ranged between 86.7-88.2%, 38.2-46.5%, 61.5-67.2%, and 90.0-94.3% for LAG-3, PD-1, SLAM, and TIM-3, respectively. CTLA-4 ranged between 8.92-15.2% and 4.10-7.29%, and CD137 was 2.80-8.30% and 7.70-17.1%, for CD4+ and CD8+ cells, respectively.

Comparison of the three TIL populations, Non-Td, anti-FOLR1 CoStAR− Td and anti-FOLR1 CoStAR+Td TILs, indicated that there was no effect of the anti-FOLR1 CoStAR on coinhibitory and costimulatory marker expression in CD4+ TILs. In CD8+ TILs, CD137+(17.1±8.79% vs 7.70±3.92% vs 7.77±4.01%) and CTLA-4+(7.29±2.41% vs 4.66±1.51% vs 4.10±1.07%) cells were of higher frequency in anti-FOLR1+Td when compared to both the non-Td and anti-FOLR1 CoStAR− Td TILs. PD-1+ TIL frequency was only higher in comparison to the anti-FOLR1− Td TILs but not the Non-Td TILs (46.5±21.1% vs 38.2±19.6% vs 40.6±26.8%, respectively). Overall, there was little observed effect of CoStAR modification of TILs for coinhibitory or costimulatory marker expression except for the slight but significant increase in the frequency of CD8+CD137+, CTLA4+, and PD-1+ TILs.

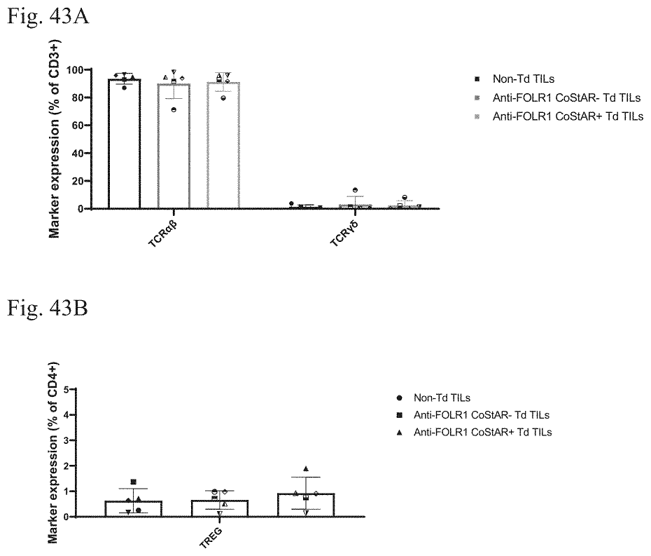

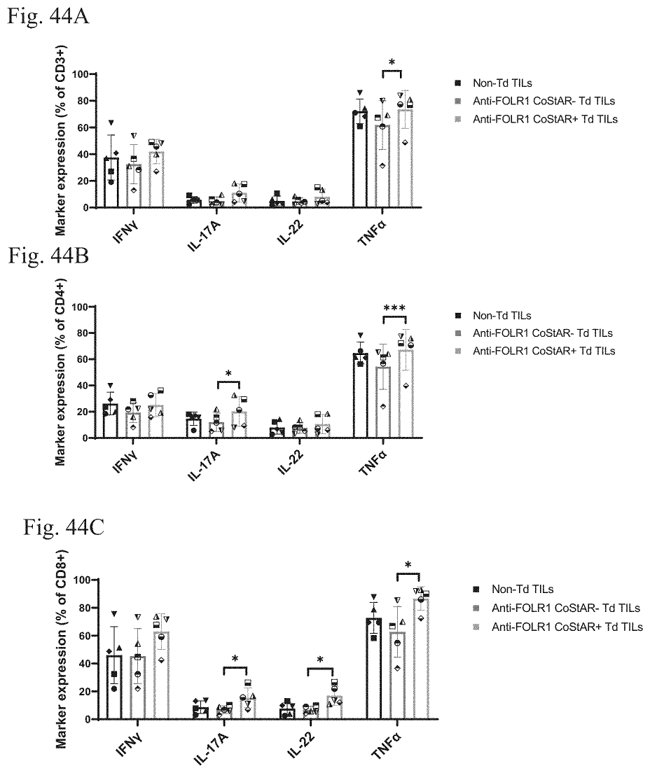

T cell subset frequency was assessed in TIL samples, using markers for Treg detection in addition to the expression of TCRαβ and TCRγδ. The majority of the CD3+ cells expressed TCRαβ (90.0-93.5%) with relatively few TCRγδ cells (1.35-3.08%) detected (FIG. 43A) and no differences were observed between the three populations. The percentage of Tregs (CD3+TCRαβ+CD4+CD25+CD127-FOXP3+) either in the CoStAR- or CoStAR+ TILs was very low. Specifically, the detection frequencies were 0.63±0.48%, 0.66±0.36% and 0.93±0.63% for non-Td, anti-FOLR1 CoStAR- and anti-FOLR1 CoStAR+Td TILs, respectively (FIG. 43B). Overall, there was no effect of anti-FOLR1 CoStAR on TCRαβ, TCRγ6, and Treg frequencies when comparing the Non-Td, anti-FOLR1 CoStAR− Td and anti-FOLR1 CoStAR+Td TILs.

TILs were assessed the ability of the cells to produce cytokines upon mitogenic activation using PMA/ionomycin. The Non-Td and Td TILs were activated for 4 hours using PMA/ionomycin and then stained for IFNγ, IL-22, IL-17A, and TNFα from CD3+, CD4+, and CD8+ cells. Upon stimulation, high frequencies of CD3+ TILs expressing TNFα (61.8-73-6%) and IFNγ (32.5-42.0%), and lower frequencies of IL-22 (4.74-8.07%) and IL-17A (5.74-11.0%) expressing cells were detected (FIG. 44A). The frequency of TNFα+ cells was higher in anti-FOLR1 CoStAR+Td compared to anti-FOLR1 CoStAR− Td TILs (73.6±14% vs 61.8±18.3%), but not Non-Td TILs (72.1±9.20%).

In CD4+ TILs, high frequencies of cells expressing TNFα (54.3-67.2%), and lower frequencies of IFNγ (19.3-26.2%), IL-22 (7.63-10.5%) and IL-17A (12.0-20.2%) expressing cells were detected (FIG. 44B). The frequencies of TNFα+(67.2±15.5% vs 54.3±17.2%) and IL-17A+ cells (20.2±11.3 vs 12.0±7.06) were higher in anti-FOLR1 CoStAR+Td relative to anti-FOLR1 CoStAR− Td TILs. No significant differences were observed in the frequency of CD4+ expressing TNFα or IL-17A when comparing either anti-FOLR1 CoStAR+/− Td TILs to Non-Td TILs.

Similar observations were made for the CD8+ TIL population, where high TNFα (62.7-86.7%) and IFNγ (45.3-63.0%), and lower IL-22 (6.80-17.0%) and IL-17A (6.93-15.3%) frequencies of positive cells were detected (FIG. 44C). In this population, higher frequencies of cytokines expressing cells were identified in the anti-FOLR1 CoStAR+Td TILs compared to anti-FOLR1 CoStAR− TILs with regards to TNFα (86.7±8.37% vs 62.7±18.2%), IL-17A (15.3±7.24% vs 6.93±2.77%), and IL-22 (17.0±6.90% vs 6.80±2.48%). Again, no significant differences were observed between either anti-FOLR1 CoStAR+/− Td TILs compared to Non-Td TILS.

The significant differences observed both in the CD4+ and CD8+ subpopulations between the IL-17A+ anti-FOLR1 CoStAR+Td TILs and anti-FOLR1 CoStAR-Td TILs were not statistically significant in the CD3+ bulk population (p=0.0527). Collectively, some statistically significant differences were observed within CD3+, CD4+, or CD8+ TIL populations in the proportion of IL-17A, IL-22, and TNFα expressing cells between anti-FOLR1 CoStAR+/− populations of Td cells. However, no significant differences were observed in the frequency of IFNγ, IL-17A, IL-22, and TNFα producing cells when comparing either Td population (ie, anti-FOLR1 CostAR−/+ fractions) to Non-Td TILs.

### Example 15

Antitumor Reactivity of CoStAR positive TILs

Complete Rapid Expansion Protocol (REP) Medium Consisted of 460 mL GIBCO custom P158718 supplemented with 40 mL human AB serum, gentamycin (10 μg/mL)/amphotericin (0.25 μg/mL) and vancomycin (50 μg/mL). Complete T cell medium (TCM) consisted of 450 mL RPMI 1640 GlutaMAX™ Supplement HEPES medium supplemented with 5 mL Penicillin-Streptomycin, 500 μL 2-Mercaptoethanol (50 mM), and 50 mL Fetal Bovine Serum (FBS).

Preparation of CoStAR Modified OC TILs for Functional Characterization

Non-Td and Td TILs from five OC samples were produced as described in ITIL-306-NC-010 and the transduction percentages are shown in Table 17.

TABLE 17
Non-Td and Td TIL donors

| | CoStAR % of | CoStAR % | |
|---|---|---|---|
| Donor | CD3 in Non-Td | of CD3 in Td | Symbol |
| 1 | 0.98 | 34.00 | ▪ |
| 2 | 0.11 | 24.33 | ● |
| 3 | 0.20 | 47.11 | ▾ |
| 4 | 0.09 | 45.65 | ▴ |
| 5 | 0.15 | 64.60 | ♦ |

TILs were either used directly after the REP or upon thaw from long-term cryopreservation (−150° C.; stored in cryoprotectant consisting of FBS with 10% DMSO). ovarian cancer TILs were thawed using a 37° C. water bath, transferred to a 50 mL Falcon tube with 10 mL of complete REP TCM, centrifuged at 400×g for 5 minutes and resuspended in complete REP TCM. Both post-REP and post-thaw TILs, were counted using the NovoCyte 3005 Flow Cytometer System following DRAQ7 dead cell exclusion and a CD2+ count and placed in T75 flasks at a density of 1×10⁶ cells/mL in complete REP TCM supplemented with 3000 IU/ml of interleukin 2 (IL-2). The cells were then placed in a 5% CO2 incubator set to 37° C. for 2-3-days. Before the assay, TILs were resuspended in complete REP TCM at a density of 1×10⁶ cells/mL without IL-2 and placed in a 5% CO2 incubator set to 37° C. overnight.

### Assessment of FOLR1 Expression by Autologous Tumor Digest.

All autologous tumor digests were thawed from long-term cryopreservation (−150° C.; stored in cryoprotectant consisting of FBS with 10% DMSO) using a 37° C. water bath, transferred to a 15 mL Falcon tube with 9 mL of complete TCM, centrifuged at 400×g for 5 minutes and resuspended in 5 mL of complete TCM. The number of viable cells in each sample was determined using the NovoCyte 3005 Flow Cytometer System following DRAQ7 dead cell exclusion, and the cells were resuspended at a concentration of 1×10⁶ cells/mL in completed TCM. 1×10⁵ autologous tumor digest cells were then analyzed according to the flow cytometry staining protocol and acquired using the Novocyte 3005 Flow Cytometer System.

The quantification of FOLR1 expression was conducted according to the following gating strategy: a cell gate (forward scatter-height [FSC]-H vs side scatter-height [SSC]-H), then a doublet exclusion gate (SSC-H vs side scatter-area [SSC-A]) followed by a dead cell exclusion gate (SSC-A vs Fixable Viability dye eFluor 450). The CD2-cells were then gated from which the frequency of the FOLR1+ cells were quantified (SSC-A vs FOLR1-PE).

### Cytokine Production Assessed by Intracellular Flow Cytometry

To measure the frequencies of cytokine producing T cells, Non-Td and anti-FOLR1 CoStAR Td TILs were cocultured at a 1:1 effector to target ratio (E:T, 1×10⁵ TILs:1×10⁵ target cells) with either autologous tumor digests, K-562, or OVCAR-3 derived engineered cell lines as targets. Cocultures took place in 96 well round bottom plates with 200 μL complete TCM supplemented with 1× Brefeldin A and were incubated in a 5% CO2 incubator set to 37° C. for 16 hours. Unstimulated TILs or target cells alone were used as negative controls, and positive control TILs were activated with 50 ng/mL phorbol-myristate-acetate (PMA) and 1 μg/mL ionomycin. All conditions were performed in triplicates.

Measurement of cytokine production was performed using a panel with antibodies against CD3, CD4, CD8, CoStAR (anti-idiotype 19.1 primary and anti-mouse IgG1 secondary antibodies), tumor necrosis factor alpha (TNFα), IL-2, and a viability stain. Following the 16-hour incubation, cocultures were harvested by centrifugation at 500×g for 4 minutes. The supernatant was discarded, and samples were analyzed by flow cytometry. Briefly, cells were labelled using 100 μl of fixable viability dye (1:1000 diluted in PBS) and incubated for 10 minutes at room temperature. Subsequently, cells were washed using BD stain buffer, centrifuged at 500×g for 4 minutes, and cell pellets were resuspended in 100 μl of BD stain buffer with FcR blocking reagent for 10 minutes at room temperature. Following the incubation step, cells were washed once with BD stain buffer and fixed using 4% paraformaldehyde (PFA) for 15 minutes at room temperature. A wash with BD perm/wash buffer, staining using the 19.1 anti-idiotype for 25 minutes at 4° C. and two more BD perm/wash buffer washes followed. Cells were then resuspended in 100 μl of BD stain buffer with CD3, CD4, CD8, anti-mouse IgG1, TNFα, and IL-2 and incubated at 4° C. for 25 minutes. Following two more wash steps using BD perm/wash buffer, cell pellets were resuspended in 100 μl of BD stain buffer for acquisition on NovoCyte 3005 Flow Cytometer System.

The quantification of TNFα and IL-2 producing cell frequencies in the CD4 and CD8 subpopulations was conducted according to a gating strategy that included dead cell exclusion. CD3+ cells were gated from the live gate (SSC-A vs CD3-A) and then CD4+ and CD8+ cells were gated from the CD3+ gate (CD4-A vs CD8-A). TNFα and IL-2 producing cells were reported from the CoStAR negative (−) subset for the Non-Td TILs, and both CoStAR− and CoStAR positive (+) fractions from the Td TILs (anti-FOLR1 CoStAR− Td TILs and anti-FOLR1 CoStAR+Td TILs).

### Cytokine Production Assessed by MSD Immunoassay

To measure cytokine secretion, non-Td and anti-FOLR1 CoStAR Td TILs were cocultured at a 1:1 ratio (E:T, 1×10⁵ TILs:1×10⁵ target cells) with autologous tumor digests and at a 8:1 ratio (E:T, 1×10⁵ TILs:1.25×10⁴ target cells) with K-562 or BA/F3 derived engineered cell lines. Cocultures were performed in 200 μL complete TCM in triplicate. In experiments where MHC blocking was conducted, autologous tumor digests were pre-incubated (4° C.) with antibodies directed against MHC Class I (HLA-ABC; 40 μg/mL), MHC Class II (HLA-DRDPDQ; 40 g/mL), MHC Class I+II (both 40 μg/mL), or an isotype control (Mouse IgG2a; 80 μg/mL) for 45 minutes in 100 μL complete TCM. Following the incubation, TILs were added to the appropriate wells. For all experiments, unstimulated TILs or target cells alone were used as negative controls, and TILs activated with 50 ng/mL phorbol-myristate-acetate (PMA) and 1 g/mL ionomycin were used as a positive control. The cells were then placed in a 5% CO2 incubator set to 37° C. for 24 hours.

Following the 24-hour incubation, samples were centrifuged at 400×g for 5 minutes, and the supernatant was harvested before storage at −80° C. Upon thaw, samples were appropriately diluted in complete TCM and Diluent 2 from the V-Plex Plus Proinflammatory Panel 1 kit from Meso scale discovery (MSD). The assay was performed per manufacturer's instructions.

### Target Cell Cytotoxicity Assessed by Flow Cytometry

To measure the cytotoxic activity of TILs, Non-Td and anti-FOLR1 CoStAR Td TILs were cocultured at a 1:1 ratio (E:T, 1×10⁵ TILs: 1×10⁵ target cells) with BA/F3 derived engineered cell lines. Cocultures were performed in 96 well round bottom plates with 200 μL complete TCM in triplicate, and incubated for 20 hours in a 5% CO2 incubator set to 37° C. TILs and target cells were cultured alone as negative controls.

Following the 20-hour coculture, samples were centrifuged at 400×g for 5 minutes, the supernatant was discarded, and samples were analyzed by flow cytometry. Briefly, cells were labelled using 100 μl of fixable viability dye (1:1000 diluted in PBS) and incubated for 10 minutes at room temperature. All washes were performed using BD stain buffer. After the incubation cells were washed, centrifuged at 500×g for 4 minutes, and cell pellets were resuspended in 100 μl of BD stain buffer with FcR Blocking reagent for 10 minutes at room temperature. Following the incubation step, cells were washed once resuspended in 100 μl of BD stain buffer with CD2 and incubated at 4° C. for 25 minutes. Cells were washed twice, and cell pellets were resuspended in 100 μl of BD stain buffer for acquisition on NovoCyte 3005 Flow Cytometer System.

Target cell counts were enumerated by flow cytometry after doublet and dead cell exclusion. CD2-cells were gated from the live gate (SSC-A vs CD2-A) and quantified by the absolute count function of the NovoCyte 3005 Flow Cytometer System.

Target Cell Cytotoxicity Assessed by xCELLigence

Assessment of target cell cytotoxicity by xCELLigence was performed according to manufacturer's instructions. Briefly, E-plates were equilibrated by adding 50 μL TCM per well, incubated at room temperature for 30 minutes, and background electrical impedance was acquired on the RTCA Analyzer (37° C., 5% CO2). Following this, 3×10⁴ OVCAR-3 derived engineered cell lines in 50 μL TCM were added per well of each E-plate and incubated at room temperature for 30 minutes before cell growth was assessed by electrical impedance (cell index) upon the RTCA Analyzer (37° C., 5% CO2). The cell index was measured every 15 minutes throughout the duration of the assay. Upon reaching confluency (between 24-31 hours), 6×10³ or 1×10³ Non-Td or Td TILs in 100 μL TCM were added to OVCAR-3 derived engineered cell lines (E:T ratios of 1:5 and 1:30, respectively). These were incubated at room temperature for 30 minutes prior to 169 hours of further cell index readings upon the RTCA Analyzer (37° C., 5% CO2). Control conditions included wells containing target cell lines alone, target cell lines with 0.5% Triton X-100 added at OC TIL loading time-point (full lysis control), and TILs alone. The normalized cell index (NCI) was determined according to manufacturer's instructions using RTCA software pro. The area under the NCI curve as extracted from RTCA software pro for the 169-hour period of OC TIL coculture with OVCAR-3 derived engineered cell lines is reported as a quantitative readout. Both the RTCA SP and DP were used. For the 1:30 ratio of Non-Td and Td TILs from 9831 and 9260 tumor digests, cocultures were run in duplicate.

### Analysis

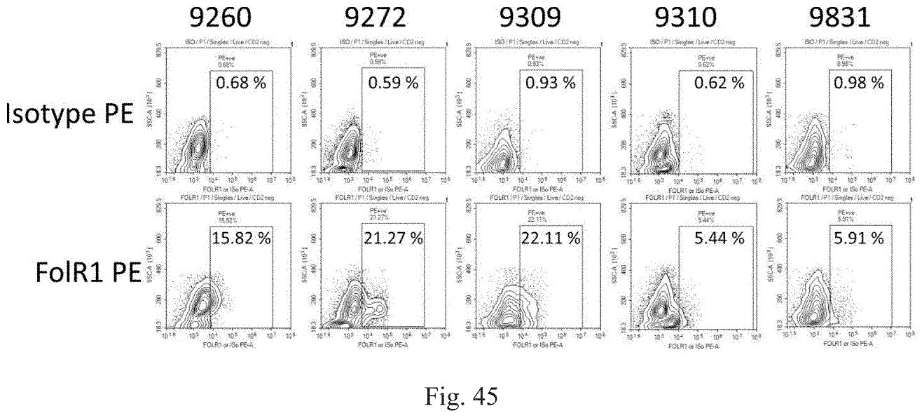

Following the production and characterization of TIL from cryopreserved tumor samples, retained input tumor digest samples were thawed and assessed for their expression of FOLR1; the ligand of anti-FOLR1 CoStAR. FOLR1 could be detected on the surface of CD2−cells in all 5 tumors with the proportion of FOLR1 positive cells ranging from 5.44-22.1% (FIG. 45).

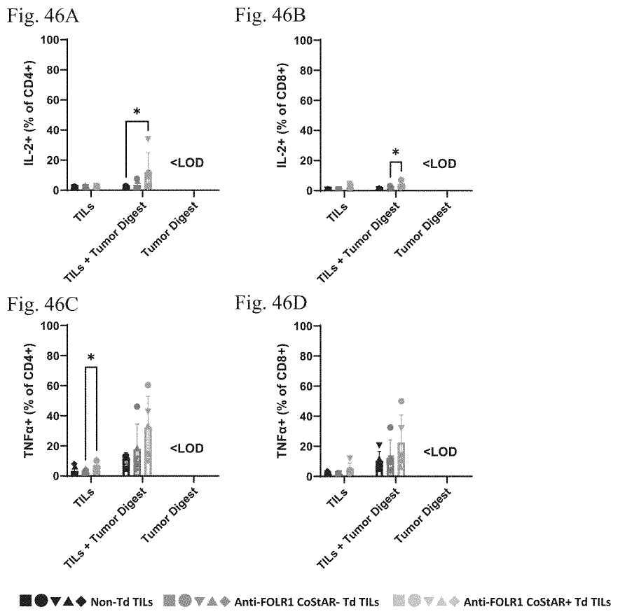

Intracellular flow cytometry was used to assess the frequency of T cells that recognize and respond to autologous tumor. PMA/I stimulation was used as a positive control for frequencies of TIL capable of readily producing IL-2 and TNFα. No IL-2+ or TNFα+ TILs were detected in tumor digests cultured overnight (16 hours) alone with brefeldin A. In contrast, IL-2+ and TNFα+ TILs were detected upon 16-hour coculture of expanded TIL with autologous tumor digest (FIG. 46). CD4+ anti-FOLR1 CoStAR+Td TILs were characterized by significantly higher frequency of IL-2 producing cells compared to Non-Td TILs with a marked 6.56-fold increase (12.0±12.9% vs 1.83±0.55%) (FIG. 46A). Although not significant, CD4+ anti-FOLR1 CoStAR+Td TILs also had a 3.33-fold increase in IL-2 producing cell frequencies compared to anti-FOLR1 CoStAR− Td TILs (12.0±12.9% vs 3.61±3.10%). A slight but significant increase in the number of anti-FOLR1 CoStAR+CD4 T cells producing TNFα (6.96±3.56%) when cultured alone relative to anti-FOLR1 CoStAR− Td TILs (2.58±1.60%), but not Non-Td TILs (3.31±3.62%) was also detected (FIG. 46C). Upon coculture with the autologous digest, TNFα+ cells seemed to increase in frequency in the anti-FOLR1 CoStAR+Td TIL population, although a statistically significant difference compared to anti-FOLR1 CoStAR− Td and Non-Td TILs was not observed (32.3% vs 17.8% vs 11.6%).

Similar observations were made for the CD8+ population upon coculture with autologous digest (FIG. 46). A significantly higher frequency of CD8+IL-2+ cells was detected by anti-FOLR1 CoStAR+Td TILs (4.13±2.26%) relative to anti-FOLR1 CoStAR− Td TILs (1.42±1.00%; 2.9-fold increase) but not the Non-Td TILs (1.11±0.55%; 4.13-fold increase, p=0.1097) (FIG. 46B). CD8+ TNFα producing cell frequencies were increased in the anti-FOLR1 CoStAR+Td TILs relative to anti-FOLR1 CoStAR-Td and Non-Td TILs, however, these differences did not reach statistical significance (22.6% vs 12.5% vs 10.5%) (FIG. 46D).

Overall, CoStAR modification significantly increased the frequencies of CD4+ and CD8+ cells producing IL-2 upon stimulation with the autologous digest. In the same setting, CD4+ and CD8+ cells frequencies producing TNFα+ trend similarly, although statistical significance was not reached.

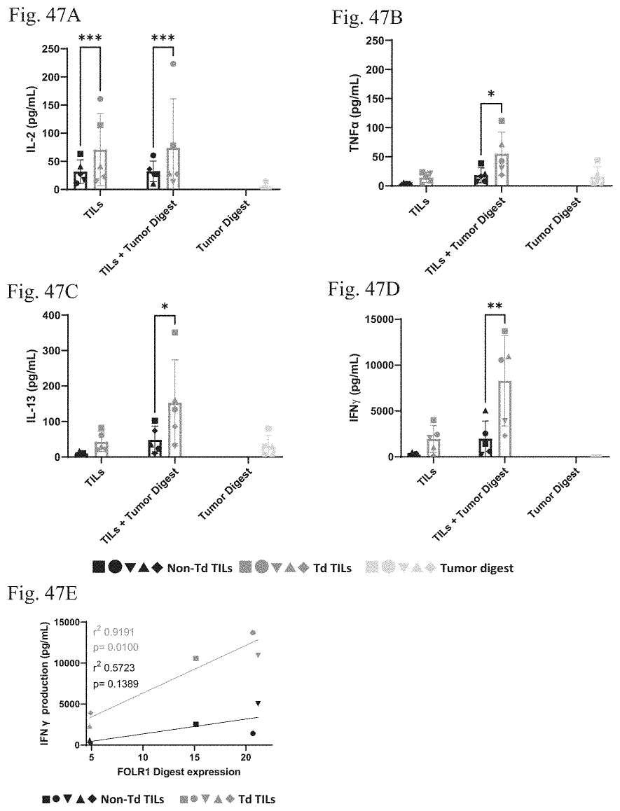

An MSD immunoassay was conducted to assess the quantity of cytokines released upon TIL coculture with autologous digest (FIG. 47). PMA/I stimulation was used as a positive control for cytokine production, which was similar between CoStAR modified and Non-Td TIL. CoStAR modification of TIL led to a statistically significant increase in the secretion of IL-2, TNFα, IL-13 and IFNγ secretion in response to autologous tumor in comparison to Non-Td TILs (FIG. 47A-D). A trend of increased background cytokines levels from Td vs Non-Td TILs when cultured alone (FIG. 47A-D) was observed and was only significant for IL-2 (70.6 pg/mL vs 31.8 pg/mL) (FIG. 47A). Upon coculture with the autologous digest, although IL-2 secretion was significantly higher in Td TILs compared to Non-Td TILs (74.1 pg/mL vs 32.1 pg/mL), this was not different from background levels of TILs cultured alone. Td TILs secreted higher levels of IFNγ in response to autologous digest compared to Non-Td TIL with production levels of 8287 pg/mL vs 1970 pg/mL (FIG. 11D). Similarly, higher levels of TNFα (55.0 pg/mL vs 18.2 pg/mL, FIG. 47B) and IL-13 (152 pg/mL vs 48.4 pg/mL, FIG. 47C) were detected when Td TILs were cocultured with the autologous digest relative to Non-Td TILs. These indicate a 3-, 4.2- and 3.1-fold increase in TNFα, IFNγ and IL-13 production by CoStAR modified Td TILs, respectively.

A statistically significant positive correlation (r²=0.9191) was observed between IFNγ released by CoStAR modified TIL upon coculture with autologous tumor and the proportion of tumor digest expressing FOLR1 (FIG. 47E). This correlation was not observed for Non-Td TILs, confirming CoStAR-FOLR1 interactions are key for TIL enhancement. Moreover, CoStAR modified TIL IFNγ release is enhanced even against ovarian tumors harboring frequencies of FOLR1 positive cells as low as 5.44% (FIG. 11E).

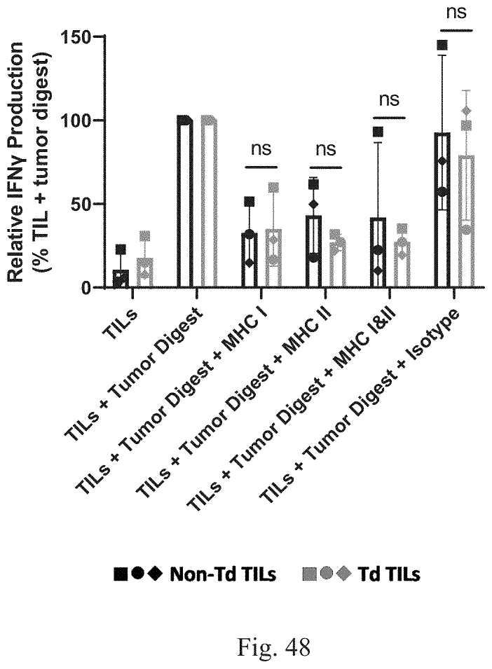

MHC blocking antibodies prevent TCRs from engaging their target and mediating TCR signaling. To assess the MHC-restricted antigen recognition of CoStAR modified TIL, Non-Td and Td TILs were cocultured with autologous tumor digest in the presence of MHC blocking, or irrelevant isotype control antibodies, and TIL anti-tumor activity was measured by IFNγ release (FIG. 48). As CoStAR modification enhanced the cytokine production in response to autologous tumor (FIG. 47), percentage reduction relative to TILs cocultured with autologous digest without blocking agents was assessed. The percentage of cytokine release was not significantly different between Non-Td and Td TILs in any of the conditions assessed. Antibodies blocking MHCI, MHCII, and both MHCI and MHCII in combination significantly decreased IFNγ release of both Non-Td (32.7%, 43.1%, and 43.1%, respectively) and Td TILs (35.0%, 27.0%, and 27.3%, respectively), relative to TIL coculture with autologous digest with no antibody present. Additionally, inhibition of cytokine release by the blocking reagents resulted in reduction that was not significantly different between any of the blocking conditions and TILs alone. Isotype control antibody had little effect on IFNγ release from Td (79.1%) or Non-Td TILs (92.7%), relative to TIL coculture with autologous digest with no antibody present. Reductions in IFNγ release from TIL by MHC blocking antibodies were statistically significant in comparison to isotype control and levels of inhibition were not statistically significantly different between Td and Non-Td TILs demonstrating equivalent dependence upon MHC-restricted antigen recognition.

Assessment of robust TIL effector function and the full potential of CoStAR to enhance these functions was demonstrated using a range of engineered cell lines (Table 18). K-562 and BA/F3 cell lines were engineered to express surface bound OKT3 (to induce a CD3 mediated signal 1) or FOLR1 (CoStAR mediated signal 2) or both OKT3 and FOLR1 (for signal 1 and 2). Additionally, the ovarian carcinoma cell line OVCAR-3, which endogenously expresses FOLR1 (63.68% FOLR1+), was engineered to express surface bound OKT3. Using these cell lines, Non-Td and Td TIL were cocultured to assess the impact of CoStAR-mediated effector functions by evaluating cytokine production and secretion by intracellular flow cytometry and MSD immunoassay, respectively.

TABLE 18
Engineered cell lines

| Parental line | Manufacturer | Derived/Engineered line |
|---|---|---|
| K-562 | ATCC | K-562 (no signal) |
| | | K-562-OKT3 (signal 1) |
| | | K-562-FOLR1 (signal 2) |
| | | K-562-OKT3-FOLR1 (signal 1 & 2) |
| OVCAR-3 | ATCC | OVCAR-3 (signal 2) |
| | | OVCAR3-OKT3 (signal 1 & 2) |
| BA/F3 | DSMZ | BA/F3 (no signal) |
| | | BA/F3-OKT3 (signal 1) |
| | | BA/F3-FOLR1 (signal 2) |
| | | BA/F3-OKT3-FOLR1 (signal 1 & 2) |

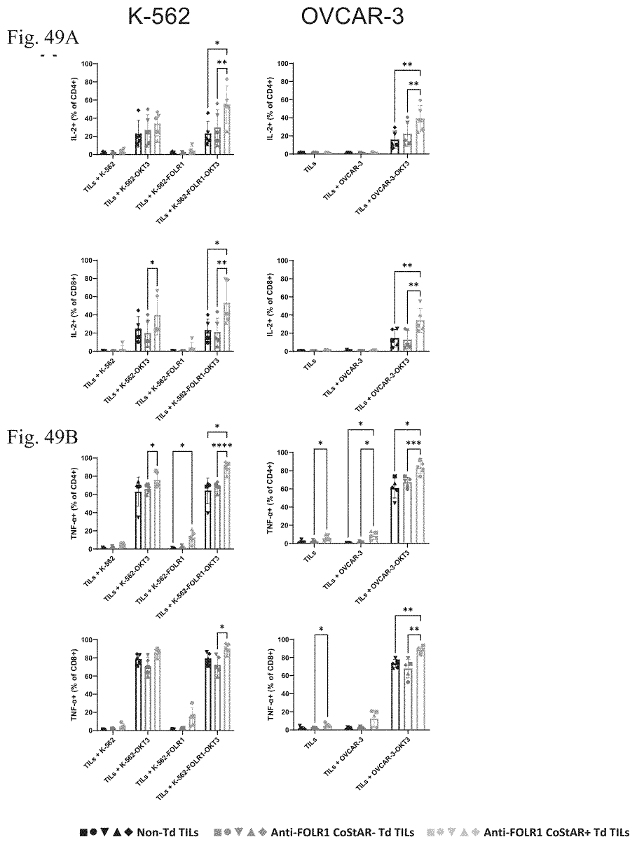

Intracellular flow cytometry was used to enumerate cytokine producing cells after 16-hour coculture with engineered K-562 and OVCAR-3 cell lines (FIG. 49). IL-2 producing cell frequencies in response to K-562 and K-562-FOLR1 were minimal for CD4+ (1.47-4.17%) and CD8+ (0.23-3.73%) cells across all populations of interest (FIG. 49A). Culture with K-562-OKT3 resulted in IL-2+ cell frequencies of 23.0-34.0% and 20.0-39.7% for CD4+ and CD8+ cell populations, respectively. No statistically significant difference in IL-2 producing cell frequencies was observed between Non-Td and anti-FOLR1 CoStAR+/− populations upon coculture with control cell lines, with the exception of CD8+ anti-FOLR1 CoStAR+Td TILs which were higher than anti-FOLR1 CoStAR− Td TILs, but not Non-Td TILs (39.7±21.7% vs 20.0±15.1% vs 24.8±13.4%, respectively). Coculture with the K-562-OKT3-FOLR1 cell line resulted in significantly higher frequencies of CD4+IL-2 producing anti-FOLR1 CoStAR+Td TILs with a marked increase of 1.84- and 2.38-fold relative to anti-FOLR1 CoStAR− Td and Non-Td TILs, respectively (54.9±20.5% vs 29.9±19.4% vs 23.1±13.4%, respectively). The same observation was true for the CD8+IL-2+ cell frequencies with a 2.53- and 2.29-fold increase in anti-FOLR1 CoStAR+Td TILs compared to anti-FOLR1 CoStAR− Td and Non-Td TILs, respectively (53.0±23.9 vs 21.0±15.5% vs 23.2±12.2%, respectively).

TILs cultured alone or cocultured with OVCAR-3 were characterized by minimal frequencies of IL-2 producing cells for both CD4+(0.78-1.38%) and CD8+ (0.22-0.80%) populations (FIG. 49A). Upon stimulation with both signals provided by the OVCAR-3-OKT3 cell line, a significant increase in the frequency of IL-2 producing cells was observed by the anti-FOLR1 CoStAR+Td TILs (39.1±14.5%) in the CD4+ population with a 1.74- and a 2.46-fold increase in comparison to anti-FOLR1 CoStAR− Td TILs (22.4±14.0%) and Non-Td TILs (15.9±9.47%), respectively. Similarly, in the CD8+ population, anti-FOLR1 CoStAR+Td TILs (34.1±13.2%) demonstrated a 2.65- and 2.36-fold increase in IL-2 producing cell frequencies compared to anti-FOLR1 CoStAR− Td TILs (12.9±10.2%) and Non-Td TILs (14.5±9.58%), respectively.

CD4+ TNFα positive cell frequencies were significantly higher in the anti-FOLR1 CoStAR+Td TILs cocultured with K-562-OKT3 compared to anti-FOLR1 CoStAR− Td TILs (75.9±8.35% vs 66.2±5.04%), and with K-562-FOLR1 compared to Non-Td TILs (12.6±7.08% vs 0.73±0.34%) (FIG. 49B). No significance was observed in the same conditions for the CD8+ TNFα positive cell populations. Stimulation with K-562-OKT3-FOLR1 expressing both signals resulted in higher frequencies of TNFα producing CD4+ cells relative to both anti-FOLR1 CoStAR− Td with 1.31-fold increase and Non-Td TILs with 1.39-fold increase (89.3±6.15% vs 68.1±4.99% vs 64.2±14.0%, respectively). The same trend was observed in the CD8+ population, however, statistical significance was only reached when comparing anti-FOLR1 CoStAR+Td TILs to anti-FOLR1 CoStAR− Td TILs (1.25-fold increase), but not the Non-Td TILs (1.14-fold increase) (90.2±5.35 vs 72.2±9.92% vs 79.3±6.30%, respectively).

In conditions where TILs were cultured alone, small but significant differences in TNFα percentage were observed between the anti-FOLR1 CoStAR+Td TILs compared to anti-FOLR1 CoStAR− Td TILs in CD4+(5.81±3.38% vs 1.85±1.42%) and CD8+(4.85±2.46% vs 1.53±0.76%) cell populations (FIG. 49B). There were no significant differences compared to Non-Td TILs in either population. In the presence of OVCAR-3 expressing signal 2, a slight but significantly higher CD4+ TNFα+ cell frequencies were detected in the anti-FOLR1 CoStAR+Td TILs relative to both the anti-FOLR1 CoStAR− Td and Non-Td TILs (8.29±4.34% vs 1.00±0.78% vs 0.40±0.23%, respectively). On the contrary, no differences were observed between these populations in the CD8+ cells. Importantly, significantly higher frequencies of CD4+ TNFα producing cells were detected upon coculture of the TILs with the OVCAR-3-OKT3 cell line in anti-FOLR1 CoStAR+Td TILs (83.5±6.99%) with a 1.24- and 1.37-fold increase compared to anti-FOLR1 CoStAR+Td TILs (67.4±5.35%) and Non-Td TILs (60.9±10.9%), respectively. Similar frequencies were detected in the CD8+ cell population, with a 1.31-fold increase between anti-FOLR1 CoStAR+Td and anti-FOLR1 CoStAR− Td TILs (88.5±3.72% vs 67.8±10.3%) and a 1.21-fold increase between anti-FOLR1 CoStAR+Td and Non-Td TILs (88.5±3.72% vs 73.2±4.57%).

Collectively, these results demonstrate a significant increase in the frequencies of IL-2+ and TNFα+ cells in the CoStAR+ fraction of the modified TILs compared to the CoStAR− fraction and Non-Td TILs when coculture with engineered cell lines expressing both signals. Although minimal, some background of higher frequencies of TNFα+ cell populations were observed in both CD4+ and CD8+ cells. Similarly, some enhancement of the CoStAR effect was observed by higher frequencies of CD8+IL-2+ and CD4+ TNFα+ cells in cocultures with K-562-OKT3 cell line, which could be potentially explained by the low levels of FOLR1 expression on the K-562.

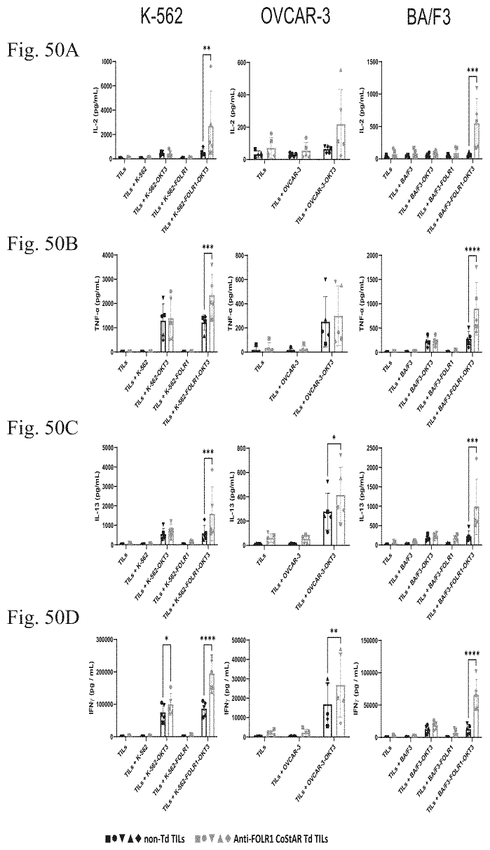

In addition to assessing the frequency of activated T cells by flow cytometry, MSD immunoassay was used to evaluate levels of secreted cytokines upon coculture with K-562 and BA/F3 engineered cell lines (Table 18). Cytokine secretion was minimal in cocultures with wild type cell lines or those engineered to express signal 2 alone (FIG. 50). IL-2 release stimulated by K-562-OKT3-FOLR1 cell lines was higher in the Td TILs (2682 pg/mL) compared to the Non-Td TILs (521.5 pg/mL) demonstrating a 5.14-fold increase (FIG. 50A). Similarly, in TILs cocultured with BA/F3-OKT3-FOLR1, IL-2 secretion was 9.31-fold higher in the Td TILs compared to Non-Td TILs (FIG. 50A). Significantly higher TNFα production was also detected in Td TILs compared to Non-Td TILs when cocultured with K-562-OKT3-FOLR1 (2337 pg/ml vs 1211 pg/mL, 1.93-fold increase) and BA/F3-OKT3-FOLR1 (900.3 pg/mL vs 278.0 pg/mL, 3.24-fold increase).

IL-13 and IFNγ production was also significantly higher upon stimulation with cell lines expressing both signals (FIG. 50C-D). More specifically, in response to stimulation with K-562-OKT3-FORL1 Td TILs secreted high levels of IL-13 (1590 pg/mL) compared with Non-Td TILs (587.0 pg/mL), showing a 2.71fold higher secretion by OKT3-FORL1 Td TILs. Similarly, a 2.26-fold higher amount of IFNγ was observed in OKT3-FORL1 Td TILs (194559 pg/mL) vs Non-Td TILs (86183 pg/mL). Similarly, IL-13 and IFNγ concentrations were significantly elevated in Td TILs cocultured with BA/F3-OKT3-FOLR1 over Non-Td TILs in the same conditions with a 4.35- (983.9 pg/mL vs 226.1 pg/mL) and 5.13-fold (65594 pg/mL vs 12786 pg/mL) greater cytokine secretion, respectively (FIG. 50C-D). Interestingly, a significantly higher IFNγ secretion was observed by Td TILs compared to Non-Td TILs in cocultures with K-562-OKT3 (1.33-fold increase, 98804 pg/mL vs 74917 pg/mL) which could be attributed to the low levels of FOLR1 expression on the K-562 cells.

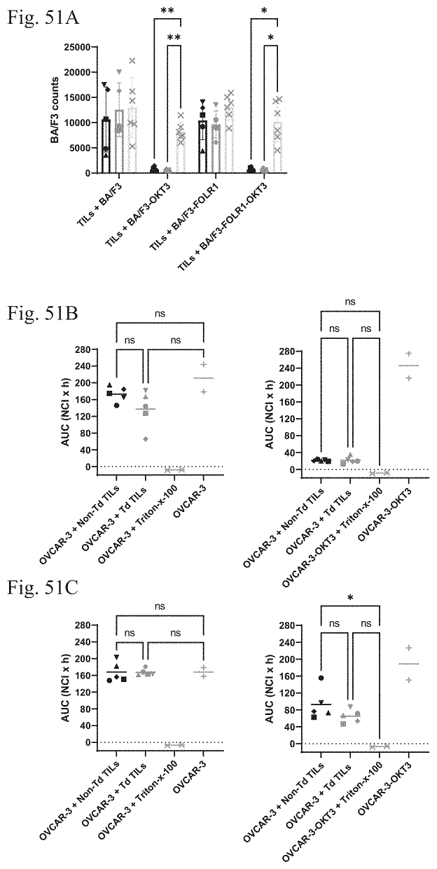

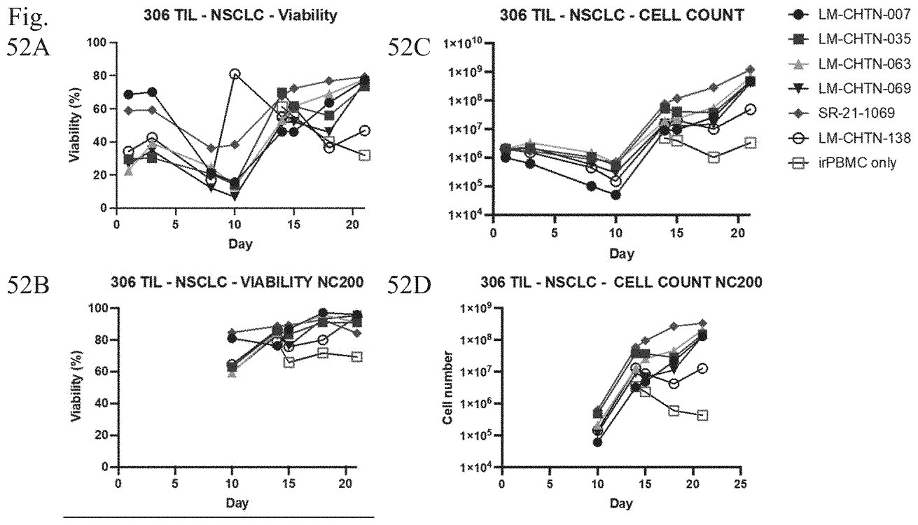

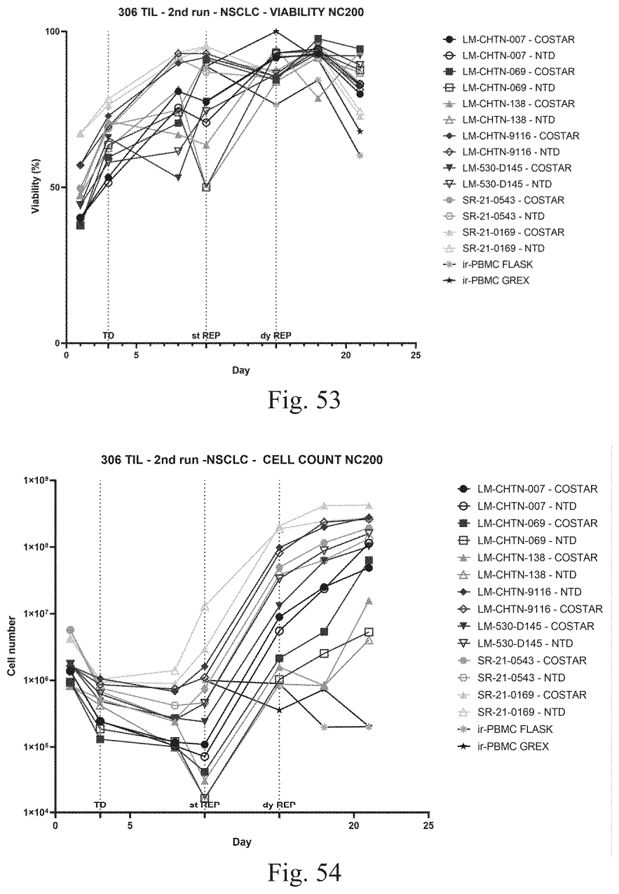

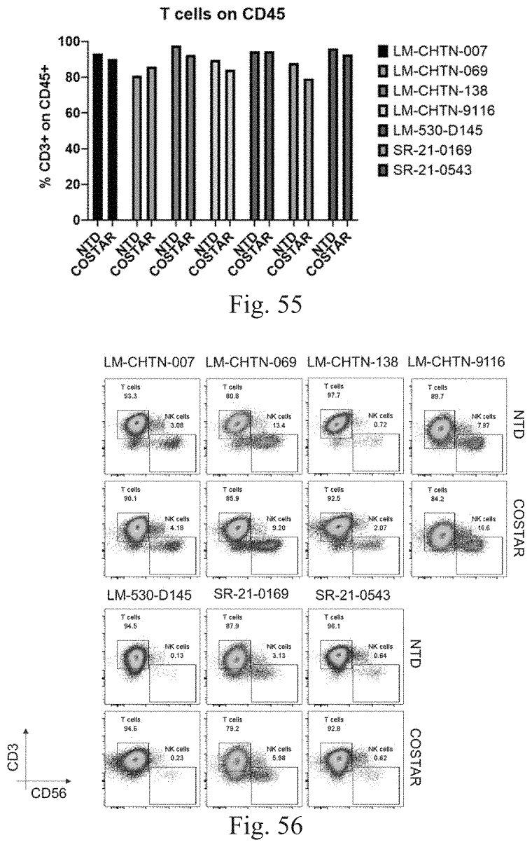

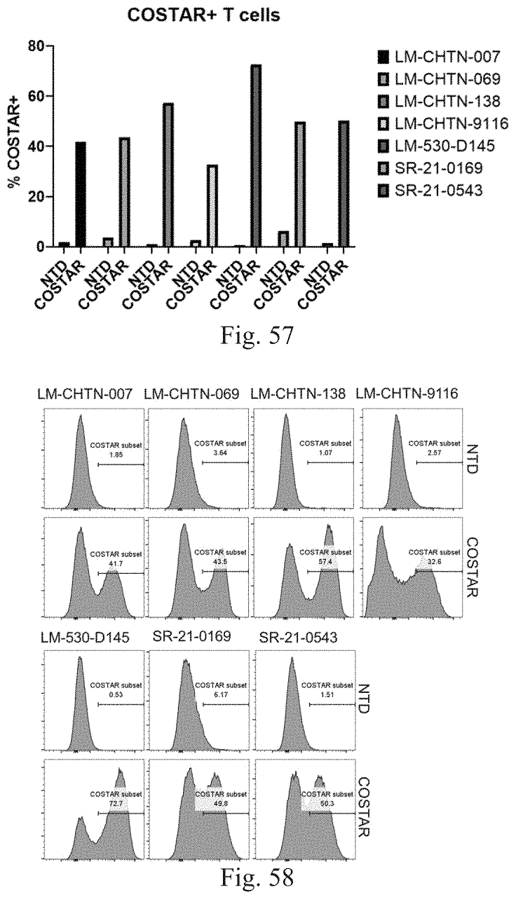

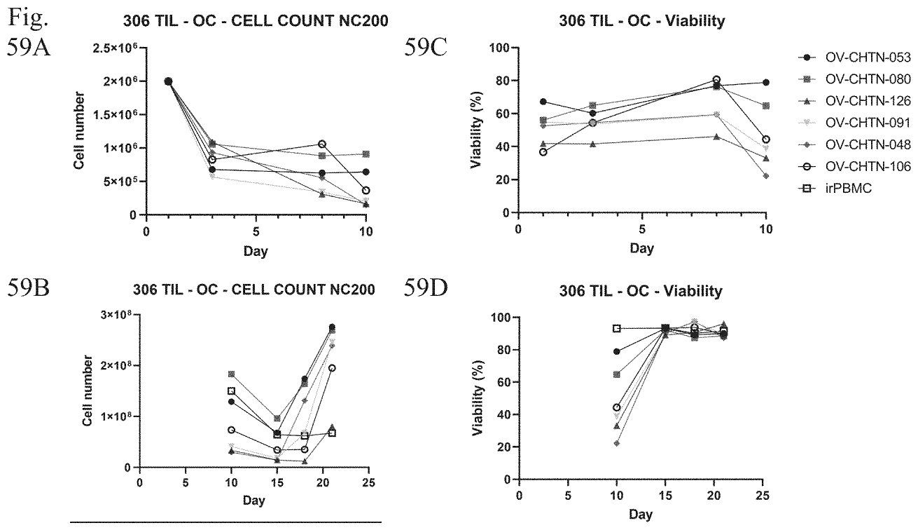

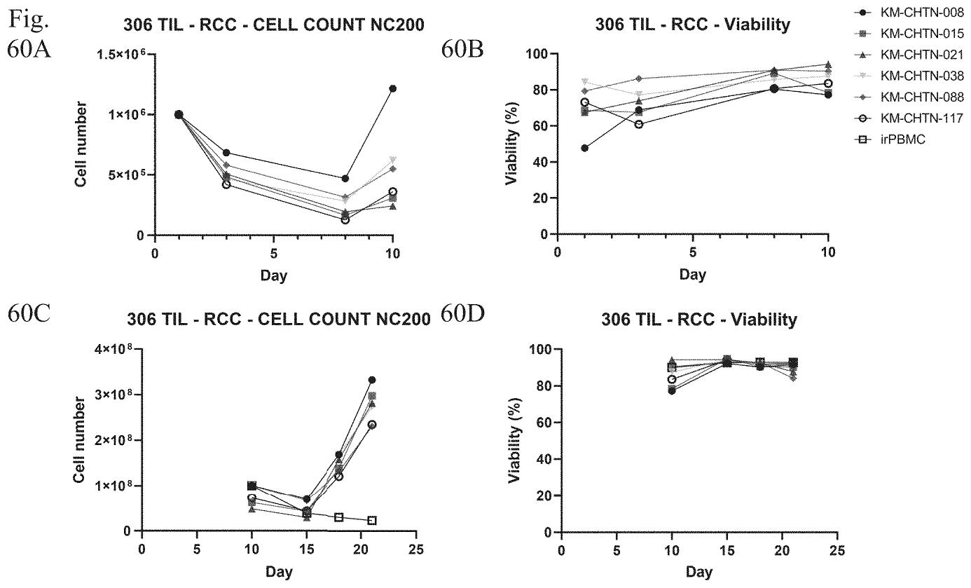

To determine the impact of CoStAR modification on the cytotoxic capacity of TILs, flow cytometry-based and xCELLigence-based killing assays were conducted against BA/F3 and OVCAR-3 engineered cell lines, respectively (FIG. 51). In cocultures assessed by flow cytometry, OKT3 was sufficient to induce cytotoxicity as Non-Td and Td TILs killed specifically BA/F3 cells expressing either OKT3 or both OKT3 and FOLF1 with no significant differences detected (FIG. 51A). Importantly, no cytotoxicity was observed against BA/F3 or BA/F3-FOLR1 demonstrating the requirement of signal 1 to induce killing. Similar observations were true for cocultures with OVCAR-3 cells as target cell lines using RTCA assays (FIG. 51B). In cocultures with OVCAR-3 OKT3 expressing both signals, there was no significant difference in the AUC (NCI×hours) between Non-Td and Td-TILs at any E:T ratio tested. OVCAR-3 cells alone were not eliminated at early timepoints, however a slight decrease in NCI of OVCAR-3 cells in 4 of 5 donors occurred following addition of Non-Td or Td TILs at an E:T 1:5 ratio at later time points.

Collectively, these data demonstrate a lack of cytotoxicity mediated by CoStAR bearing TIL against target cells expressing CoStAR target alone. Engagement of TCR was sufficient in mediating cytotoxicity in both Non-Td and Td TILs in all assays, showing no impact of CoStAR on cytotoxicity in the presence of a potent signal 1 such as OKT3.

### Example 16

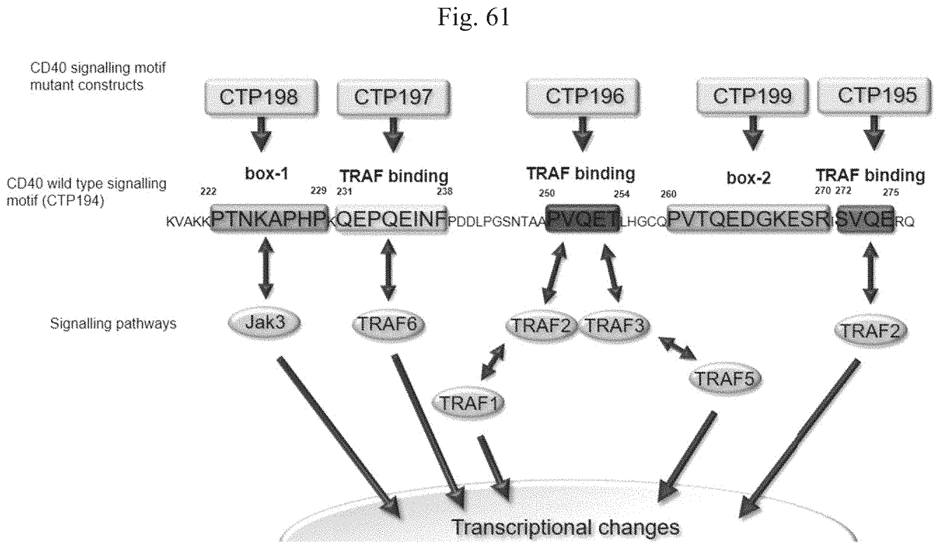

CD40 signaling motif mutant constructs CTP198, CTP197, CTP196, CTP199, and CTP195 were designed and individually incorporated into a CD40 wild type signaling motif, which contained the CD34 marker gene for sorting (FIG. 61). These constructs were then expressed in vitro, and screened for transcriptional changes compared to wild type CD40 (data not shown). From this, it was established that: (1) incorporation of CTP197 into CD40 resulted in some reduction in cell expansion, and reduction in IL-2 secretion, (2) incorporation of CTP196 into CD40 resulted in a large degree of reduction in cell expansion, and large reduction in IL-2 secretion, and (3) incorporation of CTP195 into CD40 resulted in some reduction in cell expansion, but no reduction in IL-2 secretion. From these observations, the inventors concluded that altering a CoStAR TRAF2/3 domain has a deleterious effect on cytokine production and cell proliferation. This implicates that the TRAF2/3 domain is potentially providing the majority of the CoStAR mediated activity.

TRAF domains and their influence on CoStAR signalling can be monitored by a series of mutations to either inhibit TRAF binding, inhibit expression of TRAF, modulate the TRAF binding motif, or swap TRAF domains.

### Example 17

Mutational analysis of CD40 TRAF sequence variants such as those in Table 2, 4, and 5, was conducted to further establish the function of the signaling motifs. This analysis was done in order to assess if modifying TRAF 2, 2/3 and/or 6 signalling motifs of CD40 improves proliferation and cytokine release.

TRAF domains of CD40 were swapped in the constructs as shown in Table 17, and in FIG. 67. There was a total of 12 mutant CD40 constructs generated, as well as one wild type CD40 construct, and one “mock” construct of a CoStAR without CD40.

TABLE 17

| | Construct ID | Plasmid design | Notes |
|---|---|---|---|
| | CTP343 | pSF.Lenti.MND.MOV19.CD28(TOP) | Construct without CD40 |
| | (SEQ ID NO: | | |
| | 42376) | | |
| CoStAR | CTP205 | pSF.Lenti.MND.MOV19.CD28 | Non-mutated CoStAR |
| | (SEQ ID NO: | | |
| | 42) | | |
| Traf | CTP406 | pSF.Lenti.MND.MOV19.CD28 | TRAF2/3 binding domain |
| domain | (SEQ ID NO: | (PVQE-TQEE) | targeting |
| swop | 510) | | |
| | CTP407 | pSF.Lenti.MND.MOV19.CD28 | TRAF2/3 binding domain |
| | (SEQ ID NO: | (PVQE-SKEE) | targeting |
| | 511) | | |
| | CTP408 | pSF.Lenti.MND.MOV19.CD28 | TRAF2/3 binding domain |
| | (SEQ ID NO: | (PVQE-AVEE) | targeting |
| | 512) | | |
| | CTP409 | pSF.Lenti.MND.MOV19.CD28 | TRAF2 binding domain |
| | (SEQ ID NO: | (SVQE - AVEE) | targeting |
| | 513) | | |
| | CTP410 | pSF.Lenti.MND.MOV19.CD28 | TRAF2 binding domain |
| | (SEQ ID NO: | (SVQE - PEEE) | targeting |
| | 514) | | |
| | CTP411 | pSF.Lenti.MND.MOV19.CD28 | TRAF6 binding domain |
| | (SEQ ID NO: | (PQEINF-PEEMSW) | targeting |
| | 515) | | |
| | CTP412 | pSF.Lenti.MND.MOV19.CD28 | TRAF6 binding domain |
| | (SEQ ID NO: | (PQEINF-PPENYN) | targeting |
| | 516) | | |
| | CTP413 | pSF.Lenti.MND.MOV19.CD28 | TRAF6 binding domain |
| | (SEQ ID NO: | (PQEINF-PQENSY) | targeting |
| | 517) | | |
| | CTP414 | pSF.Lenti.MND.MOV19.CD28 | TRAF2, TRAF2/3 and |
| | (SEQ ID NO: | (2-3-6 mt) | TRAF6 binding domain |
| | 518) | | mutant |
| | CTP421 | pSF.Lenti.MND.MOV19.CD28.TRAF2/3 | Contains CD28 domain |
| | (SEQ ID NO: | | plus “critical” CD40 |
| | 519) | | TRAF2/3 domain flanked |
| | | | by 5 amino acids on either |
| | | | side |

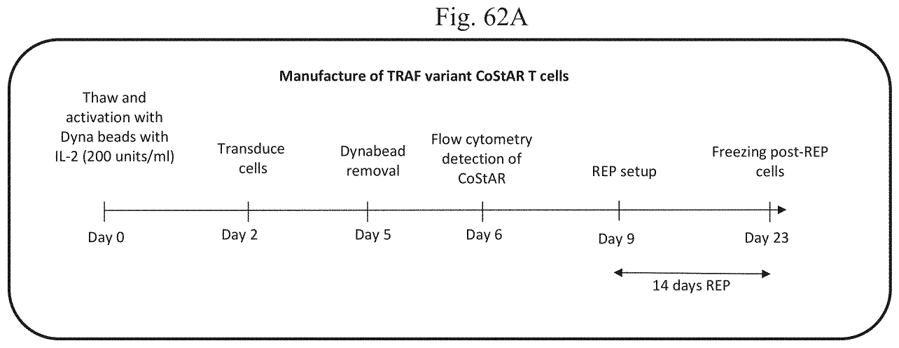

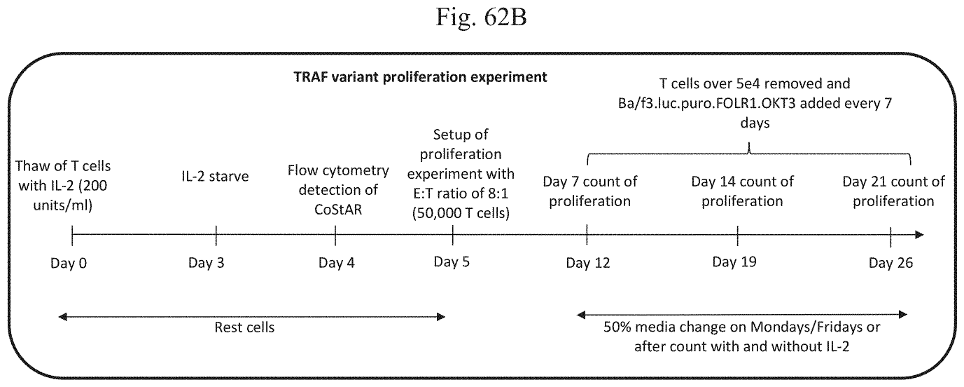

Each construct was transduced into primary human pan T cells obtained from STEM cell (#Cat 70024) from one of three donors. The proliferation of TRAF variant T cells expressing each construct was then assessed in a co-culture with Ba/f3.luc.puro.FOLR1.OKT3 at an effector to target ratio of 8:1. The proliferation experiment pipeline was conducted as shown in FIGS. 62A-62B. These experiments were conducted in 96 well plates, wherein cells were treated with or without 200 units/mL IL-2 to activate dynabeads. For the manufacturing of TRAF variant CoStAR T cells, cells were transduced after two days, dynabeads were removed at day 5, and flow cytometry was then used to detect the expression of CoStAR at day 6 (FIG. 62A). Day 9-23 then followed 14 days of REP.

For the TRAF variant proliferation experiment, cells were given 200 units/mL IL-2 for three days, followed by one day of IL-2 starvation. On day 4, flow cytometry was again used to detect expression of CoStAR. Cells were co-cultured with Ba/f3.luc.puro.FOLR1.OKT3 at an effector to target ratio of 8:1 on day 5. On days 12, 19, and 26, proliferation was assessed (as “Days 7, 14, and 21” in the figures). In addition, supernatant was collected when changing media for cytokine MSD analysis and a short term 24 co-culture experiment was set up for cytokine MSD analysis.

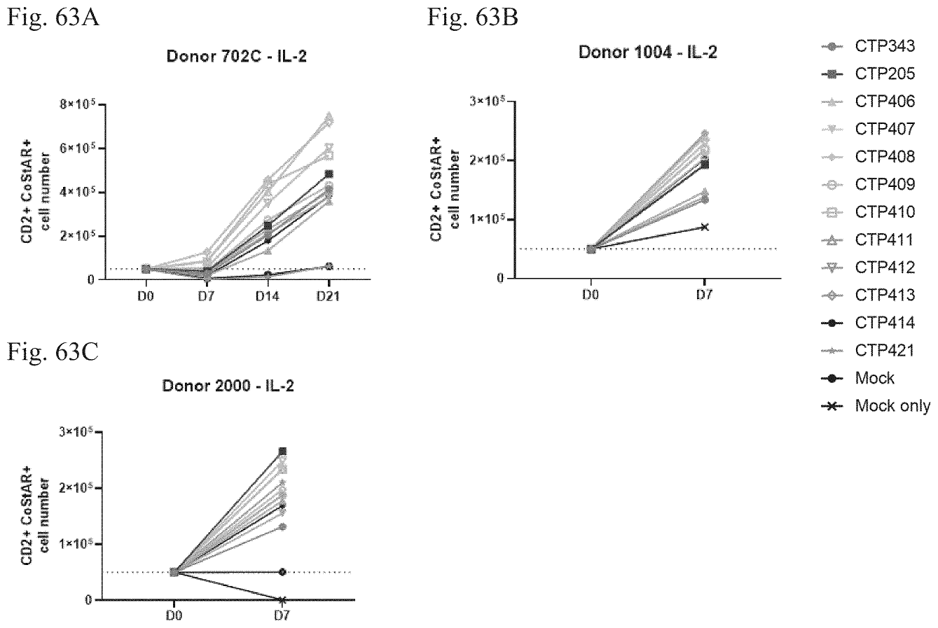

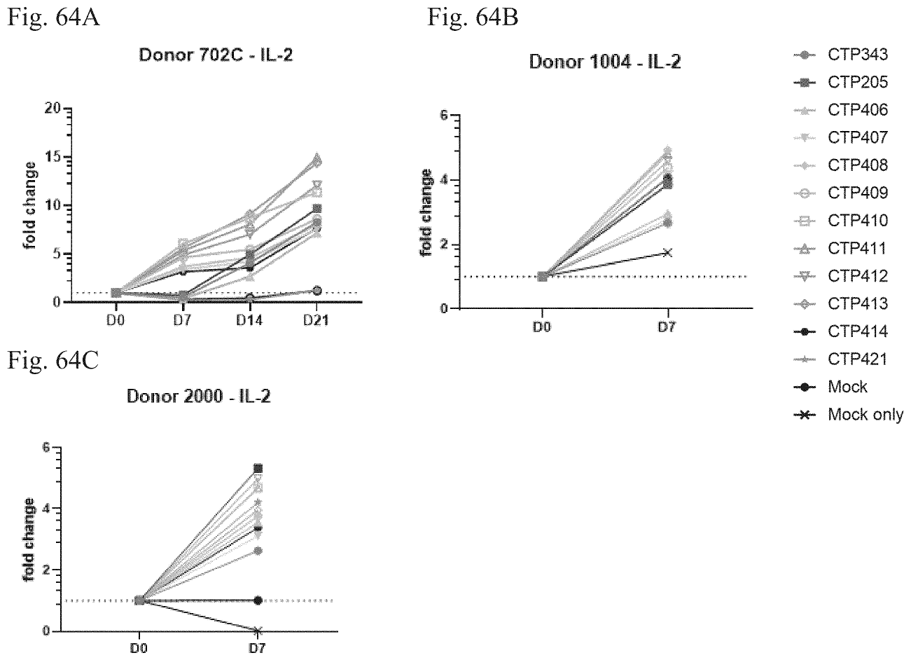

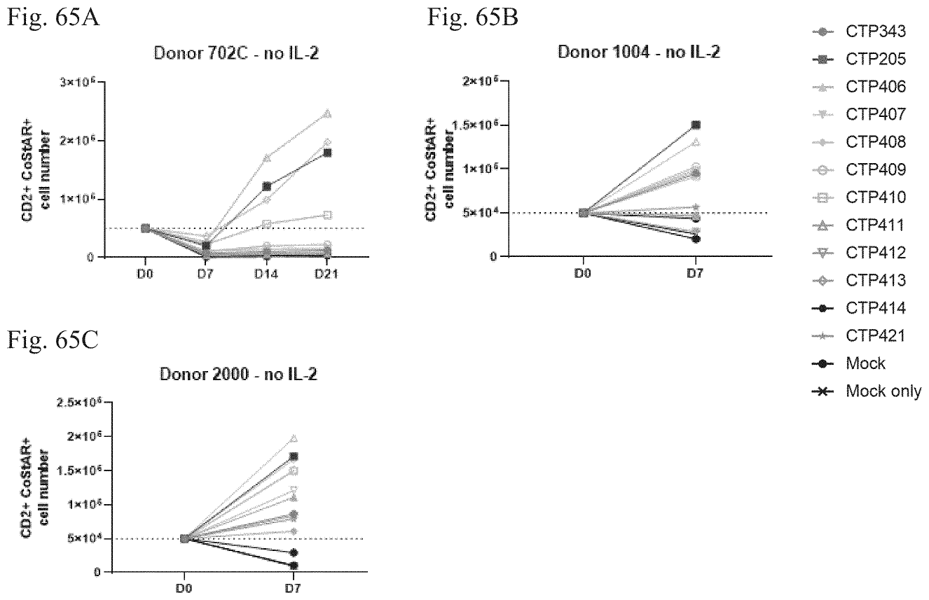

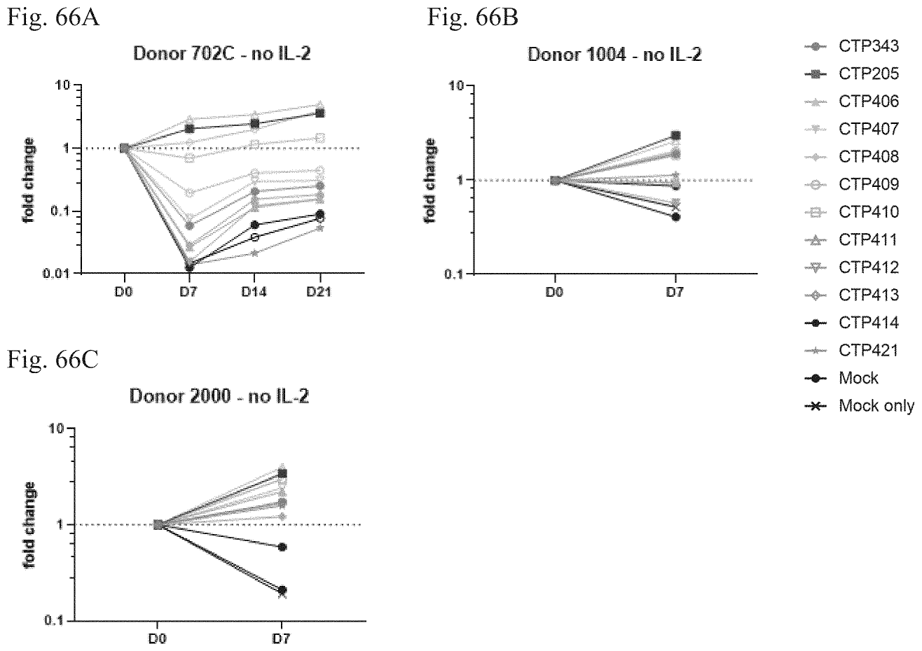

The change in total cell number and fold-change in number across the T cells from three donors (HD 702C, HD 1004, and HD 2000) with and without IL-2 treatment is as shown in FIGS. 63A-66C.

CTP411 and CTP413 (TRAF6 variants) had increased proliferation with and without IL-2 for HD donor 702C in comparison to CD28−CD40 CoSTAR (CTP205) and CD28 CoStAR (CTP343) by day 21 of the co-culture. CTP410 (TRAF2 variant) had improved proliferation in the presence of IL-2 only in comparison to CD28−CD40 CoSTAR (CTP205) by day 21. CTP408 (TRAF2/3 variant), CTP409 & CTP410 (TRAF2 variant), CTP411, CTP412 (TRAF6 variants) and CTP421 had increased proliferation in comparison to CD28−CD40 CoSTAR (CTP205) and CD28 CoStAR (CTP343) when treated with IL-2 by day 7. CD28−CD40 CoStAR (CTP205) had the highest rate of proliferation for donor HD 1004 without IL-2. CD28−CD40 CoStAR (CTP205) had the highest rate of proliferation for donor HD 2000 with IL-2. CTP411 (TRAF 6 variant) had the highest rate of proliferation for donor HD 2000 without IL-2 in comparison to CD28−CD40 CoSTAR (CTP205) and CD28 CoStAR (CTP343).

### Example 18—Production of T-Cells Expressing CoStAR

MOV19 CoStAR contains the variable heavy (VH) and variable light (VL) chains of the anti-folate receptor antibody MOV19 (Figini et al. 1998 and CAA68252.1 and CAA68253.1). The heavy and light chains were joined with a codon optimised for human nucleotide sequence encoding a S(G4S)x3 serine-glycine linker region in the orientation of VH-VL, with addition of a codon optimised for human sequence for the OSM signal peptide. Following the VL region a codon optimised for human sequence encoding a AAA(GSG)x2 motif was included. The signalling domain consists of a fusion of codon optimised for human sequences for CD28 (NP_006130.1: residues 21-220) fused directly to CD40 (NP_001241.1: residues 216-277). Constructs were gene synthesised by GenScript and cloned into pSF.Lenti (Oxford Genetics).

Lentiviral production was performed using a three-plasmid packaging system by mixing 10 μg of each packaging plasmid, plus 10 μg of the pSF.Lenti lentiviral plasmid containing the transgene, together in serum free RPMI containing 50 mM CaCl₂. The mixture was added dropwise to a 50% confluent monolayer of 293T cells in 75 cm² flasks. The viral supernatants were collected at 48 and 72 h post transfection, pooled and concentrated using LentiPac lentiviral supernatant concentration (GeneCopoeia, Rockville, Md., USA) solution according to the manufacturer's instructions. Lentiviral supernatants titrated on Jurkat T cells and then used to directly infect primary human T cells at an MOI=5 in the presence of 4 μg/ml polybrene (Sigma-Aldrich). Human peripheral blood pan T-cells (StemCell Technologies) were activated for 24 hours with Dynabeads™ Human T-activator beads (Invitrogen) according to the manufacturer's instructions before addition of lentiviral supernatants.

Cell transduction was assessed 4 days post infection using FRα.hFc protein (Acro Biosystems) and anti-IgG-PE secondary (Sigma-Aldrich). Cells were then expanded further using ×10 donor mismatched irradiated PBMC feeders at a 1:200 ratio in RPMI+10% FCS with the addition of 30 ng/mL OKT3 and 200 IU/mL IL-2 After 14 days the cells were cryopreserved ready for assay. After thaw the cells were rested for 3 days and then starved for 24-48 h in the absence of Il-2 prior to assay set up. Cells were also stained for CoStAR expression 24 h prior to assay set up.

Functionality assays were performed by mixing CoStAR positive or negative cells with Ba/F3 cells expressing a membrane anchored OKT3 single chain antibody fragment. For proliferation assays 5×10⁴ T cells were mixed with 6,250 target cells (E:T 8:1) at day 0, in the preence and absence of exogenous IL-2 (200 IU/mL) and T cell counts made after 7 days. Additional Ba/F3 target cells were added at 8:1 at day 7, with multiple rounds of stimulation performed up to day 21. Coculture assays for cytokine secretion were set up with 5×10⁴ of T cells and targets (E:T=1:1) and supernatants collected after 20 h. Cytokine secretion (TNFα, IL-2 and IFNγ) was analysed using MSD multiplexed cytokine analysis (Mesoscale Discovery) according to the manufacturer's instructions.

Primary human T cells from three donors were engineered with a MOV19.CD28.CD40 CoStAR or variants thereof. These variants consisted of domains swap CoStARs in which the CD40 TRAF2, TRAF2/3 or TRAF6 domains were replaced with TRAF domains from other TRAF domain containing human proteins, using the constructs outlined in Table 17. Additional receptors were cloned which contained either TRAF domains in the TRAF2, 2/3 and 6 regions which were assigned randomly based on the TRAF consensus sequences, CD28 followed by just the TRAF2/3 binding domain flanked by 5 C- and N-terminal amino acids, or CD28 alone (lacking the entirety of the TRAF domain containing CD40 regions assigned here as MOV19.CD28 or ΔCD40). Additional mock transduced cells were included.

Following lentivirus addition and cell manufacturing transduction levels were analysed. FIG. 68 outlines the transduction levels for each receptor and each donor. CoStAR expression was normalized to the expression of the lowest transduction efficiency in each individual donor using excess mock transduced cells. By doing this we were able to start each donor at an equivalent transduction level to mitigate any effect that expression might have on overall performance. For donor 702C this was A2-6 at 30.4%, and for donors 1004 and 2000 this was the 2-2/3-6 variant at 27.3 and 42.6% respectively.

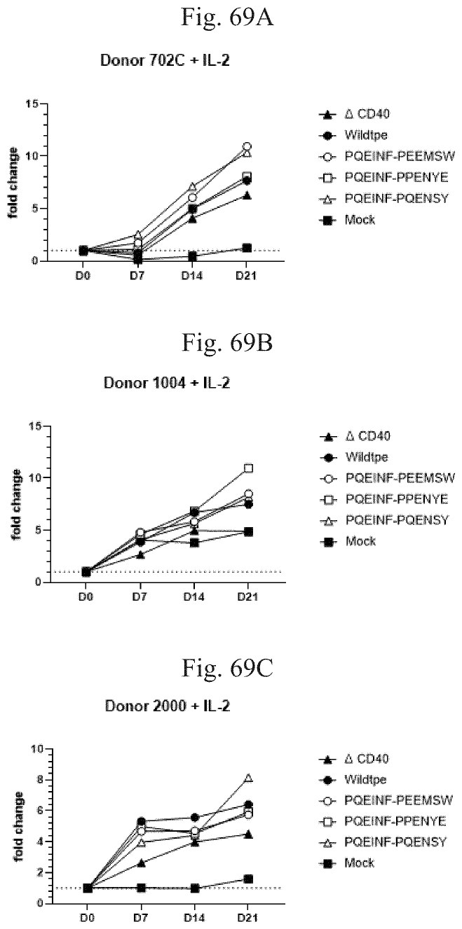

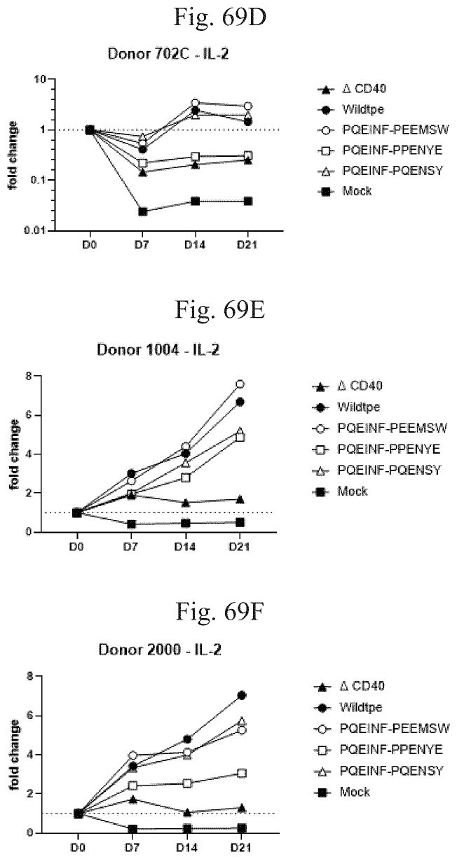

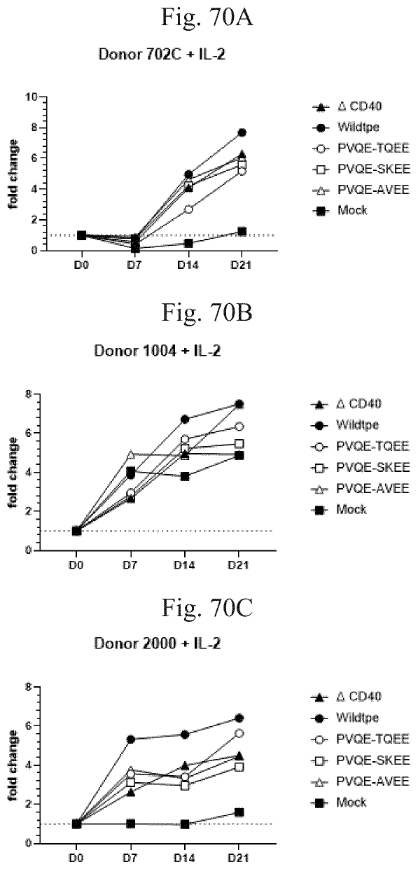

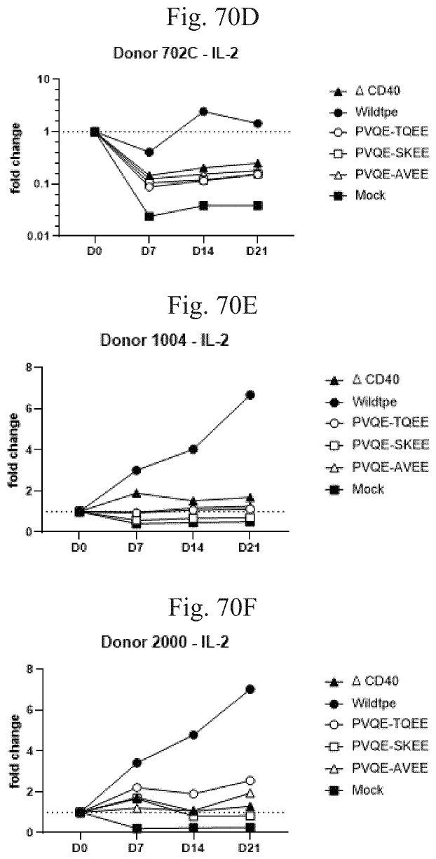

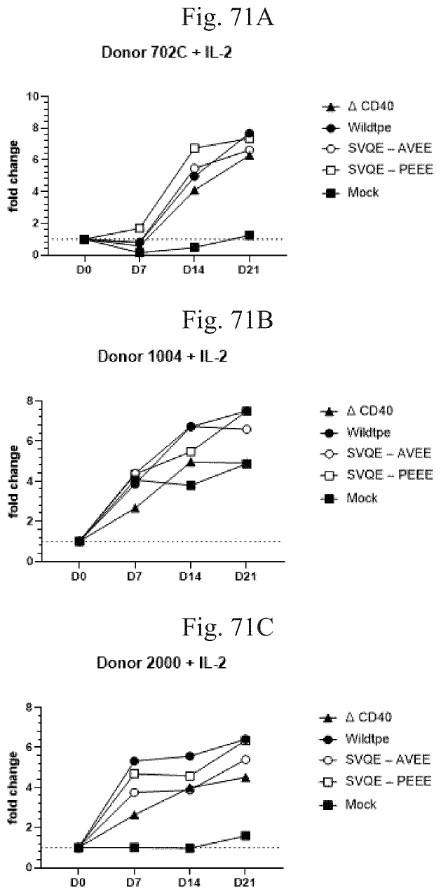

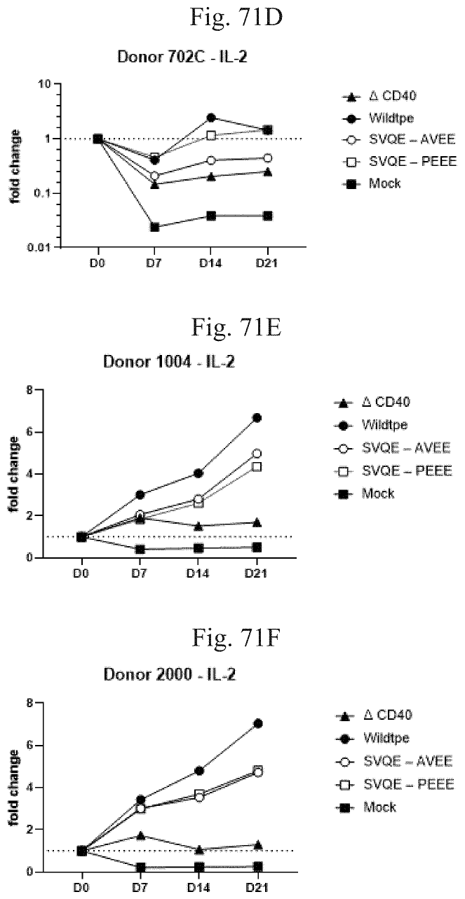

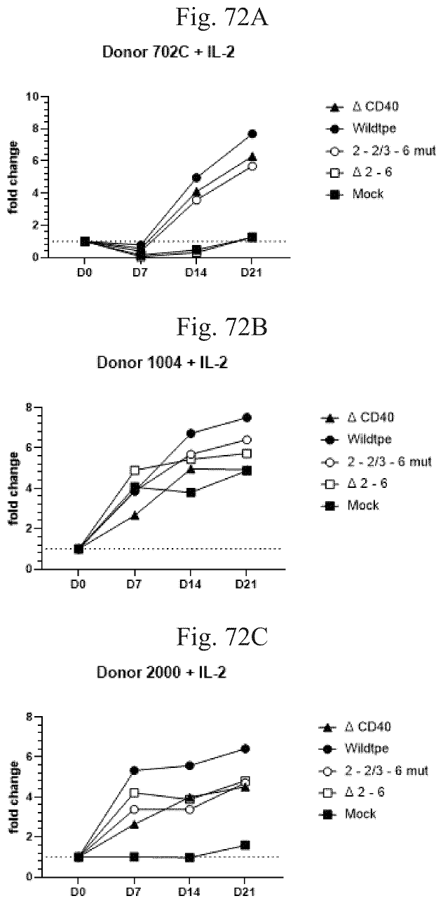

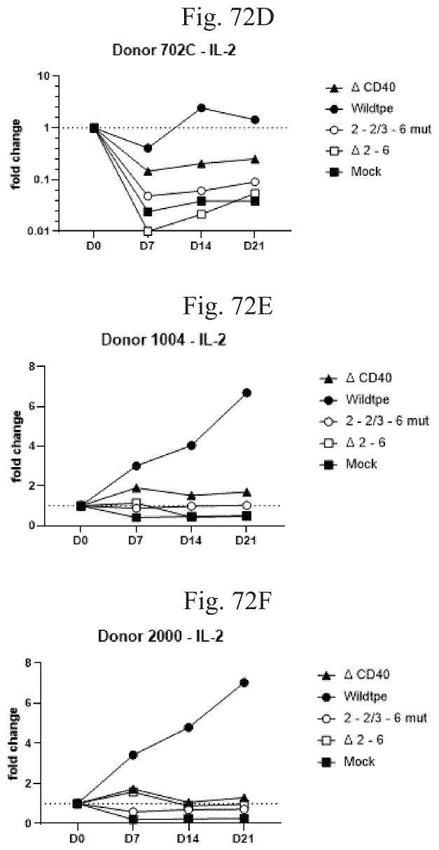

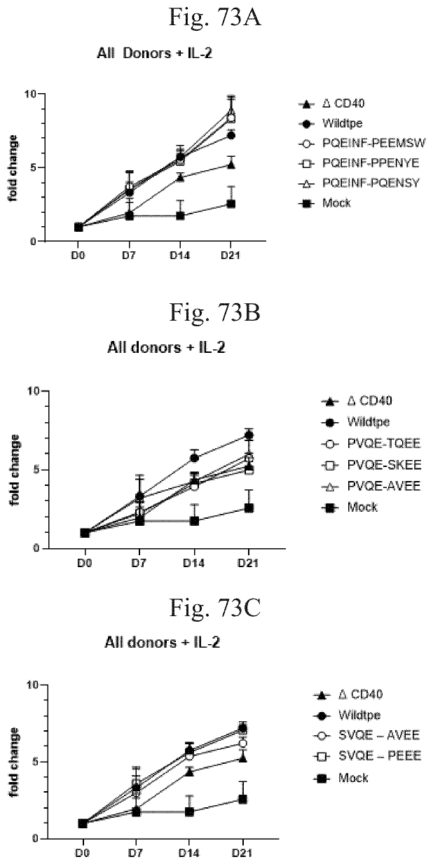

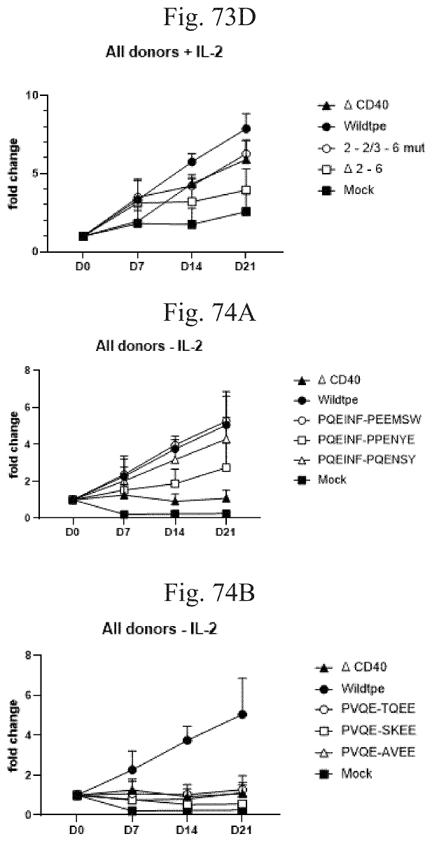

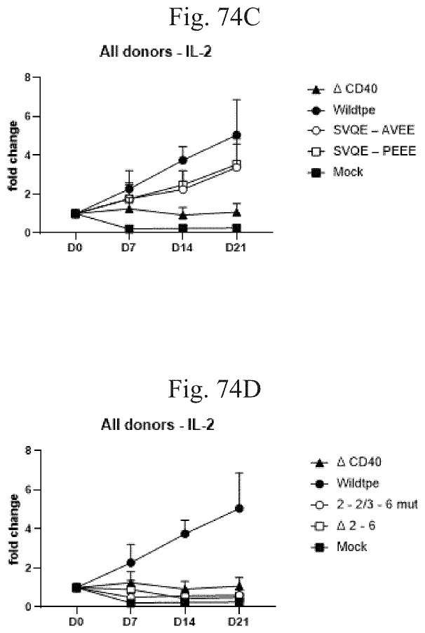

T cells were cocultured with Ba/F3-OKT3 cells at an E:T of 8:1 in the presence and absence of 200 IU/mL IL-2 and counts made at days 7, 14 and 21 in the presence and absence of exogenous IL-2. Fold change compared to base line was plotted for each donor and each receptor and also for all three donors combined. We found that swapping the TRAF6 domain did not dramatically impair the function of CoStAR compared to the wild-type receptor, and in some instances an enhancement in proliferation was observed, particularly at day 21 (FIGS. 69A-69F and 73A-73D). This effect was still observable in the absence of IL-2 but was less pronounced. Domain swaps within the TRAF2/3 domain were less well tolerated with a general reduction in proliferation seen across all three donors tested, particularly in the absence of exogenous IL-2 provision (FIGS. 70A-70F and 72A-72F). Similar results to the TRAF2/3 domain swaps were observed with the TRAF2 domain swaps, although generally the effect on proliferation was not as dramatic (FIGS. 71A-71F and 73A-73D). Finally addition of just the TRAF2/3 domain from CD40 to a basic CD28 CoStAR was not sufficient to mediate an enhancement in activity compared to the wild type receptor, and random TRAF domains based on consensus sequences were equally less efficient than the wildtype receptor and mediating T cell proliferation in the presence and absence of IL-2, particularly the latter (FIGS. 73A-73D and 74A-74D).

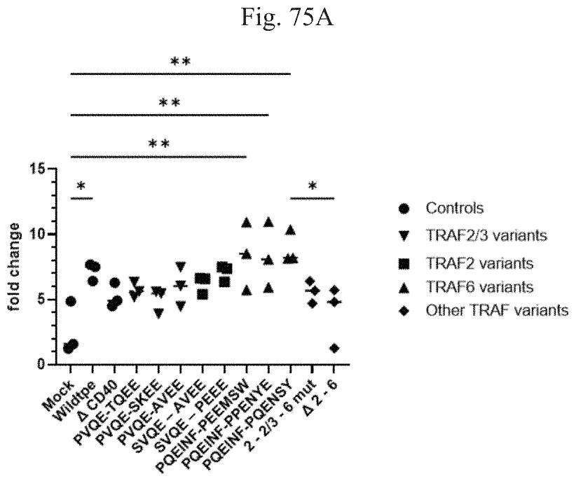

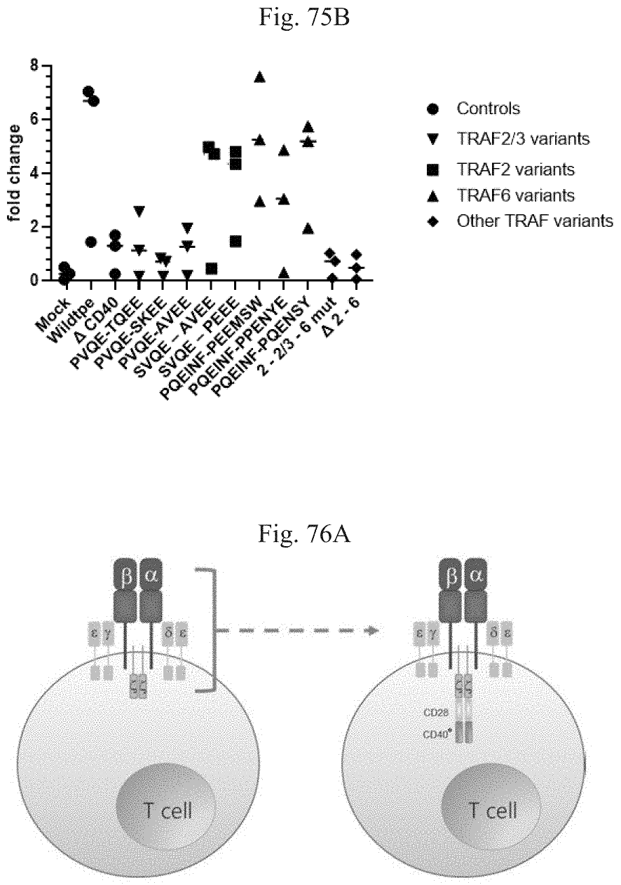

To better compare the performance of the receptors the day 21 fold change was plotted (FIGS. 75A-75B). The wild type MOV19.CD28.CD40 CoStAR as well as the three TRAF6 domain variant receptors (PQEINF-PEEMSW, PQEINF-PPENYE & PQEINF-PQENSY) significantly enhanced proliferation compared to mock transduced cells at day 21 in the presence of 11-2 (P=0.0443, 0.0045, 0.0053 & 0.0017 respectively by one-way ANOVA with post-hoc Tukey's test), with the TRAF6 variant PQENSY also demonstrating significantly enhanced proliferation compared to the A2-6 variant (P=0.0240). All other variants tested did not significantly enhance proliferation compared to mock transduced conditions. In the absence of Il-2 these trends were still observed, with the TRAF6 variants showing the best overall fold expansion along with the wild type receptor, and all TRAF2, TRAF2/3 and other TRAF variants showing little enhancement in expansion compared to mock transduced cells. Generally there was more inter-donor variability in the absence of IL-2 with all receptors tested.

### Example 19—Cytokine Production with T-Cells Expressing CoStAR

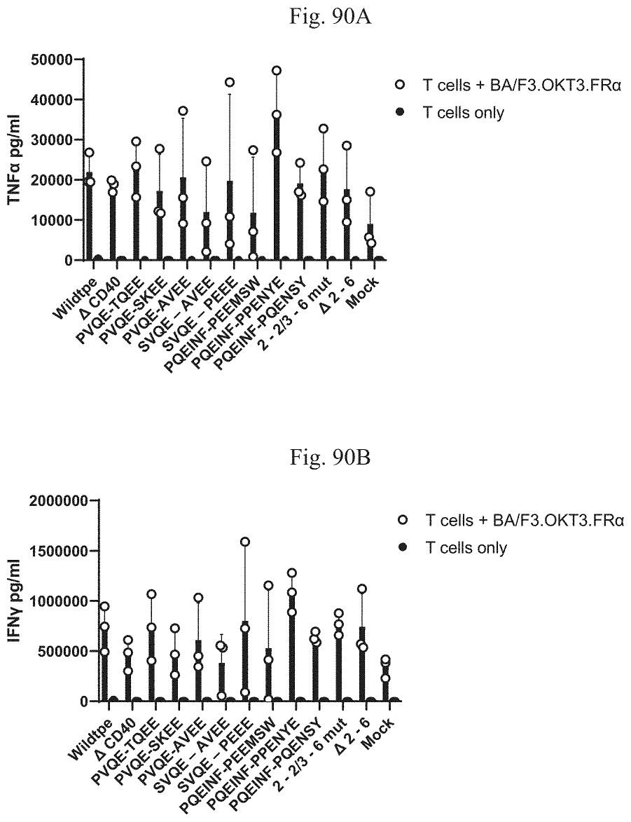

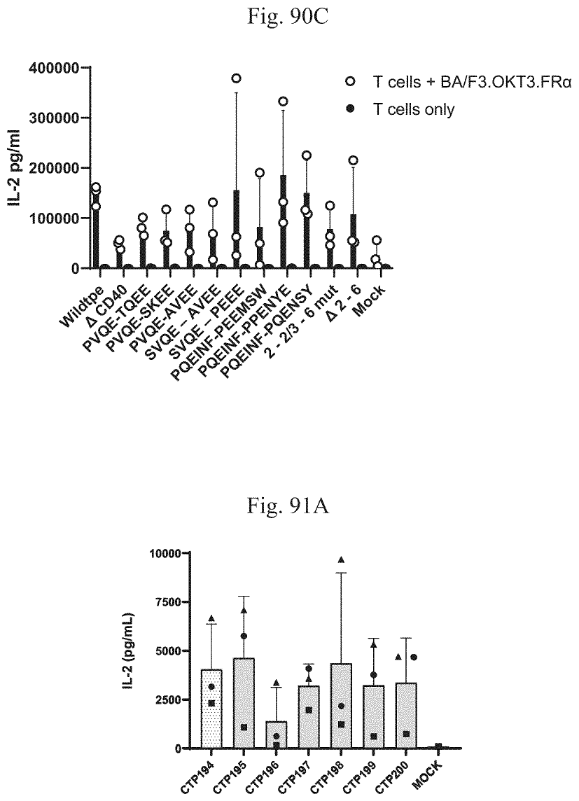

The inventors also assessed cytokine production from CoStAR variant T-cells. To this end, coculture experiments were set up between T cells and BA/F3.OKT3.FRα cells (providing signal 1 and 2). Supernatants were harvested after 20 hours and analysed for TNFα, IL-2 and IFNγ secretion by MSD (FIGS. 90A-90C). In general, wild type CD28.CD40 CoStAR+ cells induced greater cytokine production compared to mock transduced counterparts, and CoStAR containing CD28 alone (ΔCD40). The effect of the TRAF variantions was more variable but clearerst with IL-2 as a readout where variations to the TRAF2/3 domain (PVQE-TQEE, PVQE-SKEE and PVQE-AVEE) generally reduced IL-2 production compared to wild-type receptor, as did variants to the TRAF2 domain (SVQE-AVEE and SVQE-PEEE). Variations to the TRAF6 domain had more variable effects, but noticeably the PQEINF-PPENYE variant had a noticeable effect on increasing all three cytokines analysed compared to the wildtype receptor.

### Example 20—Cytokine Production with T-Cells Expressing CoStAR

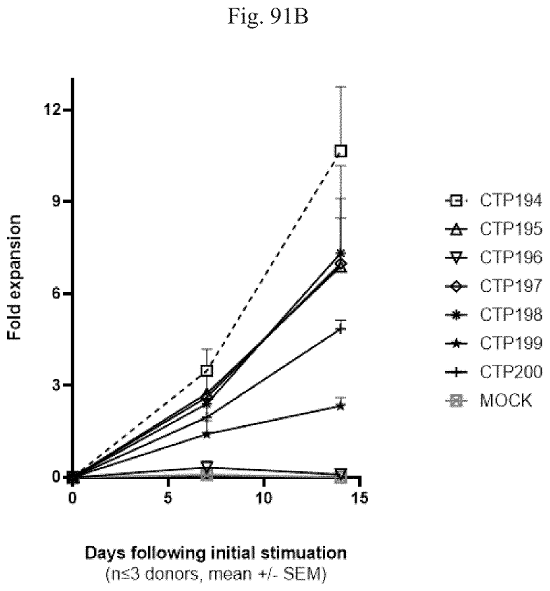

Cells from three healthy donors (NBC360, NBC362, NBC361) were activated with Dynabeads and transduced (spinoculation, MOI 5) with CTP194-CTP200 or MOCK. The cells were then magnetically enriched for their CD34 expression, expanded following the rapid expansion protocol (REP) and froze down for subsequent experiments. After thaw, cells were rested for 3-4 days in complete RPMI supplemented with IL-2 and their transduction rate was determined looking at the CD34 marker gene expression. The viability and absolute count were assessed after overnight IL-2 starvation using DRAQ-7 (1:200) by flow cytometry (Novocyte) and data were analysed using the NovoExpress 1.5.0 software. Transduced T cells were cocultured in absence of IL-2 for 6-8 days with LoVo.OKT3.GFP tumor cells at 8:1 effector to target ratio, changing half of the culture medium every 3-4 days. LoVo.OKT3.GFP naturally expresses CEA and PD-L1 on their surface, conferring both signal 2 and signal 1 (OKT3) to the transduced T cells. After 6-8 days, the viability and absolute count were assessed, and live T cells were rechallenged for an additional week with fresh LoVo.OKT3.GFP tumor cells as described above. At the end of the long-term coculture, the viability and absolute count were measured, and the fold expansion was calculated (FIG. 91B), and IL-2 levels were assessed (FIG. 91A).

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

1-24. (canceled)

25. A CD40 domain that comprises an amino acid sequence of one or more of the following:
a. A TRAF2/TRAF3 domain that comprises an amino acid sequence of Consensus A, or X₁X₂X₃X₄X₅X₆X₇X₈X₉X₁₀X₁₁, wherein:
i. X₁ is any amino acid,
ii. X₂ is any amino acid,
iii. X₃ is P, X₄ is any amino acid,
iv. X₅ is Q,
v. X₆ is C or A,
vi. X₇ is any amino acid,
vii. X₈ is any amino acid,
viii. X₉ is any amino acid,
ix. X₁₀ is any amino acid, and
x. X₁₁ is any amino acid.
b. A TRAF2/TRAF3 domain that comprises an amino acid sequence of Consensus B, or X₁X₂X₃X₄X₅X₆X₇X₈X₉X₁₀X₁₁, wherein:
i. X₁ is any amino acid,
ii. X₂ is any amino acid,
iii. X₃ is P, S, A, or T,
iv. X₄ is any amino acid,
v. X₅ is Q or E,
vi. X₆ is E,
vii. X₇ is any amino acid,
viii. X₈ is any amino acid,
ix. X₉ is any amino acid,
x. X₁₀ is any amino acid, and
xi. X₁₁ is any amino acid.
c. A TRAF 6 domain that comprises an amino acid sequence of Consensus C, or X₁X₂X₃X₄X₅X₆, wherein:
i. X₁ is P,
ii. X₂ is any amino acid,
iii. X₃ is E,
iv. X₄ is any amino acid,
v. X₅ is any amino acid, and
vi. X₆ is Ac/Ar.

26. An chimeric costimulatory antigen receptor (CoStAR) which comprises:
an extracellular binding domain that binds to carcinoembryonic antigen (CEA), or an extracellular binding domain that binds to mesothelin (MSLN);
a transmembrane domain that is linked to the extracellular binding domain, a first signaling domain; and
a second signaling domain, wherein the second signaling domain comprises a variant CD40 signaling domain or a signaling fragment thereof, wherein the variant CD40 comprises a sequence of a variant TRAF2/TRAF3 sequence, or a variant TRAF6 sequence, or a variant TRAF2 sequence, wherein the variant CD40 does not comprise SEQ ID NO: 42.

27. The CoStAR of claim 26, wherein the first signalling domain comprises a signalling domain or signaling fragment of CD28 or CD278 (ICOS).

28. The CoStAR of claim 26, wherein the first signalling domain comprises a full-length costimulatory domain.

29. The CoStAR of claim 26, wherein the extracellular binding domain is operatively linked to the transmembrane domain by a linker and/or a spacer.

30. The CoStAR of claim 29, wherein the linker comprises from about 5 to about 20 amino acids.

31. The CoStAR of claim 29, wherein the linker comprises from about 10 to about 250 amino acids.

32. The CoStAR of claim 29, wherein the transmembrane domain comprises a transmembrane domain from CD28, CD8, ICOS, DAP10, or NTRK.

33. The CoStAR of claim 29, wherein the transmembrane domain comprises the transmembrane domain sequence of SEQ ID NO:20, SEQ ID NO:21, or SEQ ID NO:22.

34. The CoStAR of claim 29, wherein the extracellular binding domain comprises an scFv, a peptide, an antibody heavy-chain variable domain, an antibody light-chain variable domain, or a CEA ligand.

35-36. (canceled)

37. A cell which expresses a CoStAR comprising:
(i) an extracellular binding domain that binds to carcinoembryonic antigen (CEA), or an extracellular binding domain that binds to mesothelin (MSLN);
a transmembrane domain that is linked to the extracellular binding domain, a first signaling domain; and
a second signaling domain, wherein the second signaling domain comprises a variant CD40 signaling domain or a signaling fragment thereof, wherein the variant CD40 comprises a sequence of a variant TRAF2/TRAF3 sequence, or a variant TRAF6 sequence, or a variant TRAF2 sequence, wherein the variant CD40 does not comprise SEQ ID NO: 42; or
(ii) a CD40 domain that comprises an amino acid sequence of one or more of the following:
a. A TRAF2/TRAF3 domain that comprises an amino acid sequence of Consensus A, or X₁X₂X₃X₄X₅X₆X₇X₈X₉X₁₀X₁₁, wherein:
i. X₁ is any amino acid,
ii. X₂ is any amino acid,
iii. X₃ is P, X₄ is any amino acid,
iv. X₅ is Q,
v. X₆ is C or A,
vi. X₇ is any amino acid,
vii. X₈ is any amino acid,
viii. X₉ is any amino acid,
ix. X₁₀ is any amino acid, and
x. X₁₁ is any amino acid.
b. A TRAF2/TRAF3 domain that comprises an amino acid sequence of Consensus B, or X₁X₂X₃X₄X₅X₆X₇X₈X₉X₁₀X₁₁, wherein:
i. X₁ is any amino acid,
ii. X₂ is any amino acid,
iii. X₃ is P, S, A, or T,
iv. X₄ is any amino acid,
v. X₅ is Q or E,
vi. X₆ is E,
vii. X₇ is any amino acid,
viii. X₈ is any amino acid,
ix. X₉ is any amino acid,
x. X₁₀ is any amino acid, and
xi. X₁₁ is any amino acid.
c. A TRAF 6 domain that comprises an amino acid sequence of Consensus C, or X₁X₂X₃X₄X₅X₆, wherein:
i. X₁ is P,
ii. X₂ is any amino acid,
iii. X₃ is E,
iv. X₄ is any amino acid,
v. X₅ is any amino acid, and
vi. X₆ is Ac/Ar.

38. The cell of claim 37, wherein the cell comprises an alpha-beta T cell, gamma-delta T cell, T regulatory cell, TIL, NKT cell or NK cell.

39. The cell of claim 37, wherein the cell coexpresses a CAR or a TCR.

40-54. (canceled)

55. The CD40 domain of claim 25, further comprising:
a. one or more of SEQ ID NOs: 475, 476, 477, 478, 479, 494, 495, 496, 498, 505, 510, 511, 512, 513, 514, 515, 516, 517, 518, or 519;
b. a sequence that is at least 70% identical to a sequence in a); or
c. a sequence that is at least 80% similar to a).

54. The CD40 domain of claim 25, wherein the TRAF6 domain sequence comprises PQEINF, PEEMSW, PPENYE, or PQENSY.

55. The CD40 domain of claim 25, wherein the TRAF2/3 domain sequence comprises PVQET, TQEET, SKEET, AVEET, or PVQET.

56. The CD40 domain of claim 25, wherein the TRAF2 domain sequence comprises SVQE, AVEE or PEEE.
